(19) United States
(12) Patent Application Publication
(10) Pub. No.: US 2005/0186569 A1
(43) Pub. Date: Aug. 25, 2005

(54) Nucleic acid-associated proteins

(76) Inventors: Shanya D. Becha, San Francisco, CA (US); Mark L. Borowsky, Needham, MA (US); Neil Burford, Durham, CT (US); Narinder K. Chawla, Union City, CA (US); Vicki S. Elliott, San Jose, CA (US); Brooke M. Emerling, Chicago, IL (US); Ian J. Forsythe, Edmonton (CA); Kimberly J. Gietzen, San Jose, CA (US); Ann E. Gorvad, Bellingham, WA (US); Jennifer A. Griffin, Fremont, CA (US); April J.A. Hafalia, Daly City, CA (US); Craig H. Ison, San Jose, CA (US); Preeti Lal, Santa Clara, CA (US); Ernestine A. Lee, Kensington, CA (US); Sally Lee, San Jose, CA (US); Soo Yeun Lee, Mountain View, CA (US); Joseph P. Marquis, San Jose, CA (US); Jayalaxmi Ramkumar, Fremont, CA (US); William W.(Webb) Sprague, Sacramento, CA (US); Anita Swarnakar, San Francisco, CA (US); Y. Tom Tang, San Jose, CA (US); Bridget A. Warren, San Marcos, CA (US); Junming Yang, San Jose, CA (US); Henry Yue, Sunnyvale, CA (US); Yeganeh Zebarjadian, San Francisco, CA (US)

Correspondence Address: INCYTE CORPORATION;EXPERIMENTAL STATION, ROUTE 141 & HENRY CLAY ROAD, BLDG. E336, WILMINGTON, DE, 19880, US

(21) Appl. No.: 10/491,472

(22) PCT Filed: Oct. 29, 2002
(86) PCT No.: PCT/US02/34846

(30) Foreign Application Priority Data
Oct. 29, 2001 (US) 60348443
Nov. 1, 2001 (US) 60335544
Nov. 5, 2001 (US) 60337535
Nov. 9, 2001 (US) 60344650
Nov. 15, 2001 (US) 60334762

(51) Int. Cl. C12Q 1/68; C07H 21/04; C12N 9/22

(52) U.S. Cl. 435/6; 435/69.1; 435/199; 435/320.1; 435/325; 530/358; 536/23.2

(57) ABSTRACT

A Various embodiments of the invention provide human nucleic acid-associated proteins (NAAP) and polynucleotides which identify and encode NAAP. Embodiments of the invention also provide expression vectors, host cells, antibodies, agonists, and antagonists. Other embodiments provide methods for diagnosing, treating, or preventing disorders associated with aberrant expression of NAAP.

### TECHNICAL FIELD

The invention relates to novel nucleic acids, nucleic acid-associated proteins encoded by these nucleic acids, and to the use of these nucleic acids and proteins in the diagnosis, treatment, and prevention of cell proliferative, neurological, developmental, and autoimmune/inflammatory disorders, and infections. The invention also relates to the assessment of the effects of exogenous compounds on the expression of nucleic acids and nucleic acid-associated proteins.

### BACKGROUND OF THE INVENTION

Multicellular organisms are comprised of diverse cell types that differ dramatically both in structure and function. The identity of a cell is determined by its characteristic pattern of gene expression, and different cell types express overlapping but distinctive sets of genes throughout development. Spatial and temporal regulation of gene expression is critical for the control of cell proliferation, cell differentiation, apoptosis, and other processes that contribute to organismal development. Furthermore, gene expression is regulated in response to extracellular signals that mediate cell-cell communication and coordinate the activities of different cell types. Appropriate gene regulation also ensures that cells function efficiently by expressing only those genes whose functions are required at a given time.

Transcription Factors

Transcriptional regulatory proteins are essential for the control of gene expression. Some of these proteins function as transcription factors that initiate, activate, repress, or terminate gene transcription. Transcription factors generally bind to the promoter, enhancer, and upstream regulatory regions of a gene in a sequence-specific manner, although some factors bind regulatory elements within or downstream of a gene coding region. Transcription factors may bind to a specific region of DNA singly or as a complex with other accessory factors. (Reviewed in Lewin, B. (1990) Genes IV, Oxford University Press, New York, N.Y., and Cell Press, Cambridge, Mass., pp. 554-570.)

The double helix structure and repeated sequences of DNA create topological and chemical features which can be recognized by transcription factors. These features are hydrogen bond donor and acceptor groups, hydrophobic patches, major and minor grooves, and regular, repeated stretches of sequence which induce distinct bends in the helix. Typically, transcription factors recognize specific DNA sequence motifs of about 20 nucleotides in length. Multiple, adjacent transcription factor-binding motifs may be required for gene regulation.

Many transcription factors incorporate DNA-binding structural motifs which comprise either α helices or β sheets that bind to the major groove of DNA. Four well-characterized structural motifs are helix-turn-helix, zinc finger, leucine zipper, and helix-loop-helix. Proteins containing these motifs may act alone as monomers, or they may form homo- or heterodimers that interact with DNA.

The helix-turn-helix motif consists of two α helices connected at a fixed angle by a short chain of amino acids. One of the helices binds to the major groove. Helix-turn-helix motifs are exemplified by the homeobox motif which is present in homeodomain proteins. These proteins are critical for specifying the anterior-posterior body axis during development and are conserved throughout the animal kingdom. The Antennapedia and Ultrabithorax proteins of Drosophila melanogaster are prototypical homeodomain proteins. (Pabo, C. O. and R. T. Sauer (1992) Annu. Rev. Biochem. 61:1053-1095.)

Mouse HES-6 is a member of the Hairy/Enhancer-of-split (HES) family of basic helix-loop-helix transcription factors. HES genes act as nuclear effectors of Notch signaling to regulate the transcriptional activity of several Notch target genes. HES-6 is expressed in all neurogenic placodes and their derivatives and in the brain, where it is patterned along both the anteroposterior and dorsoventral axes. HES-6 is also expressed in embryonic tissues where Notch signaling controls cell-fate decisions, such as the trunk, the dorsal root ganglia, myotomes, and thymus. In the limb buds HES-6 is expressed in skeletal muscle and presumptive tendons. It is also expressed in epithelial cells of the embryonic respiratory, urinary and digestive systems (Vasiliauskas, D. and Stern C. D. (2000) Mech. Dev. 98:133-137; Pissarra, L. et al. (2000) Mech Dev 95:275-278).

The zinc finger motif, which binds zinc ions, generally contains tandem repeats of about 30 amino acids consisting of periodically spaced cysteine and histidine residues. Examples of this sequence pattern, designated C2H2 and C3HC4 (“RING” finger), have been described. (Lewin, supra.) Zinc finger proteins each contain an α helix and an antiparallel β sheet whose proximity and conformation are maintained by the zinc ion. Contact with DNA is made by the arginine preceding the α helix and by the second, third, and sixth residues of the α helix. Variants of the zinc finger motif include poorly defined cysteine-rich motifs which bind zinc or other metal ions. These motifs may not contain histidine residues and are generally nonrepetitive. The zinc finger motif may be repeated in a tandem array within a protein, such that the α helix of each zinc finger in the protein makes contact with the major groove of the DNA double helix. This repeated contact between the protein and the DNA produces a strong and specific DNA-protein interaction. The strength and specificity of the interaction can be regulated by the number of zinc finger motifs within the protein. Though originally identified in DNA-binding proteins as regions that interact directly with DNA, zinc fingers occur in a variety of proteins that do not bind DNA (Lodish, H. et al. (1995) Molecular Cell Biology, Scientific American Books, New York, N.Y., pp. 447-451). For example, Galcheva-Gargova, Z. et al. (1996) Science 272:1797-1802) have identified zinc finger proteins that interact with various cytokine receptors.

The C2H2-type zinc finger signature motif contains a 28 amino acid sequence, including 2 conserved Cys and 2 conserved His residues in a C-2-C-12-H-3-H type motif. The motif generally occurs in multiple tandem repeats. A cysteine-rich domain including the motif Asp-His-His-Cys (DHHC-CRD) has been identified as a distinct subgroup of zinc finger proteins. The DHHC-CRD region has been implicated in growth and development. One DHHC-CRD mutant shows defective function of Ras, a small membrane-associated GTP-binding protein that regulates cell growth and differentiation, while other DHHC-CRD proteins probably function in pathways not involving Ras (Bartels, D. J. et al. (1999) Mol. Cell Biol. 19:6775-6787).

Zinc-finger transcription factors are often accompanied by modular sequence motifs such as the Kruppel-associated box (KRAB) and the SCAN domain. For example, the hypoalphalipoproteinemia susceptibility gene ZNF202 encodes a SCAN box and a KRAB domain followed by eight C2H2 zinc-finger motifs (Honer, C. et al. (2001) Biochim. Biophys. Acta 1517:441-448). The SCAN domain is a highly conserved, leucine-rich motif of approximately 60 amino acids found at the amino-terminal end of zinc finger transcription factors. SCAN domains are most often linked to C2H2 zinc finger motifs through their carboxyl-terminal end. Biochemical binding studies have established the SCAN domain as a selective hetero- and homotypic oligomerization domain. SCAN domain-mediated protein complexes may function to modulate the biological function of transcription factors (Schumacher, C. et al. (2000) J. Biol. Chem. 275:17173-17179).

The KRAB (Kruppel-associated box) domain is a conserved amino acid sequence spanning approximately 75 amino acids and is found in almost one-third of the 300 to 700 genes encoding C2H2 zinc fingers. The KRAB domain is found N-terminally with respect to the finger repeats. The KRAB domain is generally encoded by two exons; the KRAB-A region or box is encoded by one exon and the KRAB-B region or box is encoded by a second exon. The function of the KRAB domain is the repression of transcription. Transcription repression is accomplished by recruitment of either the KRAB-associated protein-1, a transcriptional corepressor, or the KRAB-A interacting protein. Proteins containing the KRAB domain are likely to play a regulatory role during development (Williams, A. J. et al. (1999) Mol. Cell Biol. 19:8526-8535). A subgroup of highly related human KRAB zinc finger proteins detectable in all human tissues is highly expressed in human T lymphoid cells (Bellefroid, E. J. et al. (1993) EMBO J. 12:1363-1374). The ZNF85 KRAB zinc finger gene, a member of the human ZNF91 family, is highly expressed in normal adult testis, in serninomas, and in the NT2/D1 teratocarcinoma cell line (Poncelet, D. A. et al. (1998) DNA Cell Biol.17:931-943).

The Kruppel protein regulates Drosophila segmentation. There are approximately 300 genes which encode such proteins in the whole human genome. In fact, more than 100 different mRNAs encoding Kruppel multifingered proteins, most of them novel, have been found in the human placenta. The sequences of the 106 finger repeats present in nine of these proteins are highly homologous. There are a few positions located in the alpha-helical structure which show variability. Research implies that this variability impacts the DNA-binding specificity of the proteins (Bellefroid, E. J. et al. (1989) DNA 8:377-387).

ZNF143 is a human zinc finger Kruppel family protein of the GLI type. It is 84% identical to the Xenopus laevis selenocysteine tRNA gene transcription activating factor (Staf). Staf is implicated in the enhanced transcription of small nuclear RNA (snRNA) and snRNA-type genes by RNA polymerases II (Pol II) and III (Pol III). Staf also possesses the capacity to stimulate expression from a Pol II mRNA promoter. ZNF143, along with the related ZNF138 and ZNF139, is localized to chromosome regions 7q11.2, 7q21.3-q22.1, and 11p15.3-p15.4. These regions are involved in deletion and/or translocations associated with Williams syndrome, split hand and foot disease (SHFD1), and Beckwith-Wiedemann syndrome, respectively, suggesting that ZNF143 gene is involved in developmental and malignant disorders. ZNF143 mRNAs are expressed in many normal adult tissues, including leukocytes, colon, small intestine, ovary, testis, prostate, thymus, and spleen tissues. Further, transcription of the mouse chaperone-encoding Ccta gene is regulated by ZNF143 and another Staf family zinc-finger transcription factor, ZNF76, implying that these RNA and chaperone genes are coregulated to facilitate synthesis of mature proteins during active cell growth (Tommerup, N. and Vissing, H. (1995) Genomics 27: 259-264; Myslinski, E. et al. (1998) J. Biol. Chem. 273:21998-2006; Kubota, H. et al. (2000) J. Biol. Chem. 275:28641-28648).

The C4 motif is found in hormone-regulated proteins. The C4 motif generally includes only 2 repeats. A number of eukaryotic and viral proteins contain a conserved cysteine-rich domain of 40 to 60 residues (called C3HC4 zinc-finger or RING finger) that binds two atoms of zinc, and is probably involved in mediating protein-protein interactions. The 3D “cross-brace” structure of the zinc ligation system is unique to the RING domain. The spacing of the cysteines in such a domain is C-x(2)-C-x(9 to 39)-C-x(1 to 3)-H-x(2 to 3)-C-x(2)-C-x(4 to 48)-C-x(2)-C. The PHD finger is a C4HC3 zinc-finger-like motif found in nuclear proteins thought to be involved in chromatin-mediated transcriptional regulation.

GATA-type transcription factors contain one or two zinc finger domains which bind specifically to a region of DNA that contains the consecutive nucleotide sequence GATA. NMR studies indicate that the zinc finger comprises two irregular anti-parallel β sheets and an α helix, followed by a long loop to the C-terminal end of the finger (Ominchinski, J. G. (1993) Science 261:438-446). The helix and the loop connecting the two β-sheets contact the major groove of the DNA, while the C-terminal part, which determines the specificity of binding, wraps around into the minor groove.

The LIM motif consists of about 60 amino acid residues and contains seven conserved cysteine residues and a histidine within a consensus sequence (Schmeichel, K. L. and Beckerle, M. C. (1994) Cell 79:211-219). The LIM family includes transcription factors and cytoskeletal proteins which may be involved in development, differentiation, and cell growth. One example is actin-binding LIM protein, which may play roles in regulation of the cytoskeleton and cellular morphogenesis (Roof, D. J. et al. (1997) J. Cell Biol. 138:575-588). The N-terminal domain of actin-binding LIM protein has four double zinc finger motifs with the LIM consensus sequence. The C-terminal domain of actin-binding LIM protein shows sequence similarity to known actin-binding proteins such as dematin and villin. Actin-binding LIM protein binds to F-actin through its dematin-like C-terminal domain. The LIM domain may mediate protein-protein interactions with other LIM-binding proteins.

Myeloid cell development is controlled by tissue-specific transcription factors. Myeloid zinc finger proteins (MZF) include MZF-1 and MZF-2. MZF-1 functions in regulation of the development of neutrophilic granulocytes. A murine homolog MZF-2 is expressed in myeloid cells, particularly in the cells committed to the neutrophilic lineage. MZF-2 is down-regulated by G-CSF and appears to have a unique function in neutrophil development (Murai, K et al. (1997) Genes Cells 2:581-591).

The leucine zipper motif comprises a stretch of amino acids rich in leucine which can form an anphipathic α helix. This structure provides the basis for dimerization of two leucine zipper proteins. The region adjacent to the leucine zipper is usually basic, and upon protein dimerization, is optimally positioned for binding to the major groove. Proteins containing such motifs are generally referred to as bZIP transcription factors. The leucine zipper motif is found in the proto-oncogenes Fos and Jun, which comprise the heterodimeric transcription factor AP1 involved in cell growth and the determination of cell lineage (Papavassiliou, A. G. (1995) N. Engl. J. Med. 332:45-47).

The mouse kreisler (kr) mutation causes segmentation abnormalities in the caudal hindbrain and defective inner ear development. The kr cDNA encodes a basic domain-leucine zipper (bZIP) transcription factor in which a serine is substituted for an asparagine residue conserved in the DNA-binding domain of all known bZIP family members. The identity, expression, and mutant phenotype of kr indicate an early role in axial patterning and provide insights into the molecular and embryologic mechanisms that govern hindbrain and otic development (Cordes, S. P. and Barsh, G. S. (1994) Cell 79:1025-1034).

The helix-loop-helix motif (HLH) consists of a short α helix connected by a loop to a longer α helix. The loop is flexible and allows the two helices to fold back against each other and to bind to DNA. The transcription factor Myc contains a prototypical HLH motif.

The NF-kappa-B/Rel signature defines a family of eukaryotic transcription factors involved in oncogenesis, embryonic development, differentiation and immune response. Most transcription factors containing the Rel homology domain (RHD) bind as dimers to a consensus DNA sequence motif termed kappa-B. Members of the Rel family share a highly conserved 300 amino acid domain termed the Rel homology domain. The characteristic Rel C-terminal domain is involved in gene activation and cytoplasmic anchoring functions. Proteins known to contain the RHD domain include vertebrate nuclear factor NF-kappa-B, which is a heterodimer of a DNA-binding subunit and the transcription factor p65, mammalian transcription factor ReIB, and vertebrate proto-oncogene c-rel, a protein associated with differentiation and lymphopoiesis (Kabrun, N. and Enrietto, P. J. (1994) Semin. Cancer Biol. 5:103-112).

A DNA binding motif termed ARID (AT-rich interactive domain) distinguishes an evolutionarily conserved family of proteins. The approximately 100-residue ARID sequence is present in a series of proteins strongly implicated in the regulation of cell growth, development, and tissue-specific gene expression. ARID proteins include Bright (a regulator of B-cell-specific gene expression), dead ringer (involved in development), and MRF-2 (which represses expression from the cytomegalovirus enhancer) (Dallas, P. B. et al. (2000) Mol. Cell Biol. 20:3137-3146).

The ELM2 (Egl-27 and MTA1 homology 2) domain is found in metastasis-associated protein MTA1 and protein ER1. The Caenorhabditis elegans gene egl-27 is required for embryonic patterning MTA1, a human gene with elevated expression in metastatic carcinomas, is a component of a protein complex with histone deacetylase and nucleosome remodelling activities (Solari, F. et al. (1999) Development 126:2483-2494). The ELM2 domain is usually found to the N terminus of a myb-like DNA binding domain. ELM2 is also found associated with an ARID DNA.

LEF-1 is a transcription factor that participates in the regulation of the T-cell receptor alpha (TCR alpha) enhancer by facilitating the assembly of multiple proteins into a higher order nucleoprotein complex. The function of LEF-1 is dependent, in part, on the HMG domain. This domain induces a sharp bend in the DNA helix and on an activation domain that stimulates transcription only in a specific context of other enhancer-binding proteins. ALY is a LEF-1-interacting protein which is a ubiquitously expressed, nuclear protein that specifically associates with the activation domains of LEF-1 and AML-1 (acute myeloid leukemia 1). AML-1 is another protein component of the TCR alpha enhancer complex. ALY increases DNA binding by both LEF-1 and AML proteins. Overexpression of ALY stimulates the activity of the TCR alpha enhancer complex in transfected nonlymphoid HeLa cells, whereas down-regulation of ALY by anti-sense oligonucleotides eliminates TCR alpha enhancer activity in T cells. Similar to LEF-1, ALY can stimulate transcription in the context of the TCR alpha enhancer but apparently not when tethered to DNA through an heterologous DNA-binding domain. Research suggests that ALY mediates context-dependent transcriptional activation by facilitating the functional collaboration of multiple proteins in the TCR alpha enhancer complex (Bruhn, L. et al. (1997) Genes Dev. 11:640-653).

A family of nuclear proteins, designated SL3-3 enhancer factors 2 (SEF2), interact with an Ephrussi box-like motif within the glucocorticoid response element in the enhancer of the murine leukemia virus SL3-3. Mutation of the DNA sequence decreased the basal enhancer activity in various cell lines. The important nucleotides for binding of SEF2 are conserved in most type C retroviruses. Various cell types displayed differences both in the sets of SEF2-DNA complexes formed and in their amounts. A cDNA which encoded a protein, SEF2-1A, that interacted specifically with the SEF2-binding sequence has been isolated from human thymocytes. The nucleotide sequence specificity of the recombinant SER2-1A, expressed in Escherichia coli, corresponds to that of one of the nuclear SEF2 proteins (Corneliussen, B. et al. J(1991) J. Virol. 65:6084-6093).

The Iroquois (Irx) family of genes are found in nematodes, insects and vertebrates. Irx genes usually occur in one or two genomic clusters of three genes each and encode transcriptional controllers that possess a characteristic homeodomain. The Irx genes function early in development to specify the identity of diverse territories of the body. Later in development in both Drosophila and vertebrates, the Irx genes function again to subdivide those territories into smaller domains. (For a review of Iroquois genes, see Cavodeassi, F. et al. (2001) Development 128:2847-2855.) For example, mouse and human Irx4 proteins are 83% conserved and their 63-aa homeodomain is more than 93% identical to that of the Drosophila Iroquois patterning genes. lrx4 transcripts are predominantly expressed in the cardiac ventricles. The homeobox gene Irx4 mediates ventricular differentiation during cardiac development (Bruneau, B. G. et al. (2000) Dev. Biol. 217:266-77).

Histidine triad (HIT) proteins share residues in distinctive dimeric, 10-stranded half-barrel structures that form two identical purine nucleotide-binding sites. Hint (histidine triad nucleotide-binding protein)-related proteins, found in all forms of life, and fragile histidine triad (Fhit)-related proteins, found in animals and fungi, represent the two main branches of the HIT superfamily. Fhit homologs bind and cleave diadenosine polyphosphates. Fhit-Ap(n)A complexes appear to function in a proapoptotic tumor suppression pathway in epithelial tissues (Brenner C. et al. (1999) J. Cell Physiol.181:179-187).

Most transcription factors contain characteristic DNA binding motifs, and variations on the above motifs and new motifs have been and are currently being characterized. (Faisst, S. and S. Meyer (1992) Nucleic Acids Res. 20:3-26.)

Chromatin Associated Proteins

In the nucleus, DNA is packaged into chromatin, the compact organization of which limits the accessibility of DNA to transcription factors and plays a key role in gene regulation. (Lewin, supra, pp. 409-410.) The compact structure of chromatin is determined and influenced by chromatin-associated proteins such as the histones, the high mobility group (HMG) proteins, and the chromodomain proteins. There are five classes of histones, H1, H2A, H2B, H3, and H4, all of which are highly basic, low molecular weight proteins. The fundamental unit of chromatin, the nucleosome, consists of 200 base pairs of DNA associated with two copies each of H2A, H2B, H3, and H4. H1 links adjacent nucleosomes. HMG proteins are low molecular weight, non-histone proteins that may IS play a role in unwinding DNA and stabilizing single-stranded DNA. Chromodomain proteins play a key role in the formation of highly compacted heterochromatin, which is transcriptionally silent.

The SWI/SNF complex in yeast facilitates the function of transcriptional activators by opposing chromatin-dependent repression of transcription. In mammals SWI/SNF complexes are present in multiple forms made up of 9-12 proteins known as BRG1-associated factors (BAFs) ranging from 47 to 250 kD. BRG1-associated factors (BAFs) include the SWI2-SNF2 homolog which interacts with and activates human immunodeficiency virus integrase and is homologous to the yeast SNF5 gene (Wang, W. et al. (1996) Genes Dev. 10:2117-2130).

Diseases and Disorders Related to Gene Regulation

Many neoplastic disorders in humans can be attributed to inappropriate gene expression. Malignant cell growth may result from either excessive expression of tumor promoting genes or insufficient expression of tumor suppressor genes. (Cleary, M. L. (1992) Cancer Surv. 15:89-104.) The zinc finger-type transcriptional regulator WT1 is a tumor-suppressor protein that is inactivated in children with Wilm's tumor. The oncogene bc1-6, which plays an important role in large-cell lymphoma, is also a zinc-finger protein (Papavassiliou, A. G. (1995) N. Engl. J. Med. 332:45-47). Chromosomal translocations may also produce chimeric loci that fuse the coding sequence of one gene with the regulatory regions of a second unrelated gene. Such an arrangement likely results in inappropriate gene transcription, potentially contributing to malignancy. In Burkitt's lymphoma, for example, the transcription factor Myc is translocated to the immunoglobulin heavy chain locus, greatly enhancing Myc expression and resulting in rapid cell growth leading to leukemia (Latchman, D. S. (1996) N. Engl. J. Med. 334:28-33).

In addition, the immune system responds to infection or trauma by activating a cascade of events that coordinate the progressive selection, amplification, and mobilization of cellular defense mechanisms. A complex and balanced program of gene activation and repression is involved in this process. However, hyperactivity of the immune system as a result of improper or insufficient regulation of gene expression may result in considerable tissue or organ damage. This damage is well-documented in immunological responses associated with arthritis, allergens, heart attack, stroke, and infections. (Isselbacher et al. Harrison's Principles of Internal Medicine, 13/e, McGraw Hill, Inc. and Teton Data Systems Software, 1996.) The causative gene for autoimmune polyendocrinopathy-candidiasis-ectodermal dystrophy (APECED) was recently isolated and found to encode a protein with two PHD-type zinc finger motifs (Bjorses, P. et al. (1998) Hum. Mol. Genet. 7:1547-1553).

Furthermore, the generation of multicellular organisms is based upon the induction and coordination of cell differentiation at the appropriate stages of development. Central to this process is differential gene expression, which confers the distinct identities of cells and tissues throughout the body. Failure to regulate gene expression during development could result in developmental disorders. Human developmental disorders caused by mutations in zinc finger-type transcriptional regulators include: urogenital developmental abnormalities associated with WT1; Greig cephalopolysyndactyly, Pallister-Hall syndrome, and postaxial polydactyly type A (GL13), and Townes-Brocks syndrome, characterized by anal, renal, limb, and ear abnormalities (SALL1) (Engelkamp, D. and V. van Heyningen (1996) Curr. Opin. Genet. Dev. 6:334-342; Kohlhase, J. et al. (1999) Am. J. Hum. Genet. 64:435-445).

Human acute leukemias involve reciprocal chromosome translocations that fuse the ALL-1 gene located at chromosome region 11q23 to a series of partner genes positioned on a variety of human chromosomes. The fused genes encode chimeric proteins. The AF17 gene encodes a protein of 1093 amino acids, containing a leucine-zipper dimerization motif located 3′ of the fusion point and a cysteine-rich domain at the N terminus that shows homology to a domain within the protein Br140 (peregrin) (Prasad R. et al. (1994) Proc. Natl. Acad. Sci. U S A 91:8107-8111).

Synthesis of Nucleic Acids

Polymerases

DNA and RNA replication are critical processes for cell replication and function. DNA and RNA replication are mediated by the enzymes DNA and RNA polymerase, respectively, by a “templating” process in which the nucleotide sequence of a DNA or RNA strand is copied by complementary base-pairing into a complementary nucleic acid sequence of either DNA or RNA. However, there are fundamental differences between the two processes.

DNA polymerase catalyzes the stepwise addition of a deoxyribonucleotide to the 3′-OH end of a polynucleotide strand (the primer strand) that is paired to a second (template) strand. The new DNA strand therefore grows in the 5′ to 3′ direction (Alberts, B. et al. (1994) The Molecular Biology of the Cell, Garland Publishing Inc., New York, N.Y., pp 251-254). The substrates for the polymerization reaction are the corresponding deoxynucleotide triphosphates which must base-pair with the correct nucleotide on the template strand in order to be recognized by the polymerase. Because DNA exists as a double-stranded helix, each of the two strands may serve as a template for the formation of a new complementary strand. Each of the two daughter cells of a dividing cell therefore inherits a new DNA double helix containing one old and one new strand. Thus, DNA is said to be replicated “semiconservatively” by DNA polymerase. In addition to the synthesis of new DNA, DNA polymerase is also involved in the repair of damaged DNA as discussed below under “Ligases.”

In contrast to DNA polymerase, RNA polymerase uses a DNA template strand to “transcribe” DNA into RNA using ribonucleotide triphosphates as substrates. Like DNA polymerization, RNA polymerization proceeds in a 5′ to 3′ direction by addition of a ribonucleoside monophosphate to the 3′-OH end of a growing RNA chain. DNA transcription generates messenger RNAs (mRNA) that carry information for protein synthesis, as well as the transfer, ribosomal, and other RNAs that have structural or catalytic functions. In eukaryotes, three discrete RNA polymerases synthesize the three different types of RNA (Alberts, supra, pp. 367-368). RNA polymerase I makes the large ribosomal RNAs, RNA polymnerase II makes the mRNAs that will be translated into proteins, and RNA polymerase III makes a variety of small, stable RNAs, including 5S ribosomal RNA and the transfer RNAs (tRNA). In all cases, RNA synthesis is initiated by binding of the RNA polymerase to a promoter region on the DNA and synthesis begins at a start site within the promoter. Synthesis is completed at a stop (termination) signal in the DNA whereupon both the polymerase and the completed RNA chain are released.

Ligases

DNA repair is the process by which accidental base changes, such as those produced by oxidative damage, hydrolytic attack, or uncontrolled methylation of DNA, are corrected before replication or transcription of the DNA can occur. Because of the efficiency of the DNA repair process, fewer than one in a thousand accidental base changes causes a mutation (Alberts, supra, pp. 245-249). The three steps common to most types of DNA repair are (1) excision of the damaged or altered base or nucleotide by DNA nucleases, (2) insertion of the correct nucleotide in the gap left by the excised nucleotide by DNA polymerase using the complementary strand as the template and, (3) sealing the break left between the inserted nucleotide(s) and the existing DNA strand by DNA ligase. In the last reaction, DNA ligase uses the energy from ATP hydrolysis to activate the 5′ end of the broken phosphodiester bond before forming the new bond with the 3′-OH of the DNA strand. In Bloom's syndrome, an inherited human disease, individuals are partially deficient in DNA ligation and consequently have an increased incidence of cancer (Alberts, supra p. 247).

Nucleases

Nucleases comprise enzymes that hydrolyze both DNA (DNase) and RNA (Rnase). They serve different purposes in nucleic acid metabolism. Nucleases hydrolyze the phosphodiester bonds between adjacent nucleotides either at internal positions (endonucleases) or at the terminal 3′ or 5′ nucleotide positions (exonucleases). A DNA exonuclease activity in DNA polymerase, for example, serves to remove improperly paired nucleotides attached to the 3′-OH end of the growing DNA strand by the polymerase and thereby serves a “proofreading” function. As mentioned above, DNA endonuclease activity is involved in the excision step of the DNA repair process.

RNases also serve a variety of functions. For example, RNase P is a ribonucleoprotein enzyme which cleaves the 5′ end of pre-tRNAs as part of their maturation process. RNase H digests the RNA strand of an RNA/DNA hybrid. Such hybrids occur in cells invaded by retroviruses, and RNase H is an important enzyme in the retroviral replication cycle. Pancreatic RNase secreted by the pancreas into the intestine hydrolyzes RNA present in ingested foods. RNase activity in serum and cell extracts is elevated in a variety of cancers and infectious diseases (Schein, C. H. (1997) Nat. Biotechnol. 15:529-536). Regulation of RNase activity is being investigated as a means to control tumor angiogenesis, allergic reactions, viral infection and replication, and fungal infections.

Modification of Nucleic Acids

Methylases

Methylation of specific nucleotides occurs in both DNA and RNA, and serves different functions in the two macromolecules. Methylation of cytosine residues to form 5-methyl cytosine in DNA occurs specifically in CG sequences which are base-paired with one another in the DNA double-helix. The pattern of methylation is passed from generation to generation during DNA replication by an enzyme called “maintenance methylase” that acts preferentially on those CG sequences that are base-paired with a CG sequence that is already methylated. Such methylation appears to distinguish active from inactive genes by preventing the binding of regulatory proteins that “turn on” the gene, but permiting the binding of proteins that inactivate the gene (Alberts, supra pp. 448-451). In RNA metabolism, “tRNA methylase” produces one of several nucleotide modifications in tRNA that affect the conformation and base-pairing of the molecule and facilitate the recognition of the appropriate mRNA codons by specific tRNAs. The primary methylation pattern is the dimethylation of guanine residues to form N,N-dimethyl guanine.

Helicases and Single-Stranded Binding Proteins

Helicases are enzymes that destabilize and unwind double helix structures in both DNA and RNA. Since DNA replication occurs more or less simultaneously on both strands, the two strands must first separate to generate a replication “fork” for DNA polymerase to act on. Two types of replication proteins contribute to this process, DNA helicases and single-stranded binding proteins. DNA helicases hydrolyze ATP and use the energy of hydrolysis to separate the DNA strands. Single-stranded binding proteins (SSBs) then bind to the exposed DNA strands, without covering the bases, thereby temporarily stabilizing them for templating by the DNA polymerase (Alberts, supra, pp. 255-256).

RNA helicases also alter and regulate RNA conformation and secondary structure. Like the DNA helicases, RNA helicases utilize energy derived from ATP hydrolysis to destabilize and unwind RNA duplexes. The most well-characterized and ubiquitous family of RNA helicases is the DEAD-box family, so named for the conserved B-type ATP-binding motif which is diagnostic of proteins in this family. Over 40 DEAD-box helicases have been identified in organisms as diverse as bacteria, insects, yeast, amphibians, mammals, and plants. DEAD-box helicases function in diverse processes such as translation initiation, splicing, ribosome assembly, and RNA editing, transport, and stability. Examples of these RNA helicases include yeast Drs1 protein, which is involved in ribosomal RNA processing; yeast TIF1 and TIF2 and mammalian eIF-4A, which are essential to the initiation of RNA translation; and human p68 antigen, which regulates cell growth and division (Ripmaster, T. L. et al. (1992) Proc. Natl. Acad. Sci. USA 89:11131-11135; Chang, T.-H. et al. (1990) Proc. Natl. Acad. Sci. USA 87:1571-1575). These RNA helicases demonstrate strong sequence homology over a stretch of some 420 amino acids. Included among these conserved sequences are the consensus sequence for the A motif of an ATP binding protein; the “DEAD box” sequence, associated with ATPase activity; the sequence SAT, associated with the actual helicase unwinding region; and an octapeptide consensus sequence, required for RNA binding and ATP hydrolysis (Pause, A. et al. (1993) Mol. Cell Biol. 13:6789-6798). Differences outside of these conserved regions are believed to reflect differences in the functional roles of individual proteins (Chang, T. H. et al. (1990) Proc. Natl. Acad. Sci. USA 87:1571-1575).

Some DEAD-box helicases play tissue- and stage-specific roles in spermatogenesis and embryogenesis. Overexpression of the DEAD-box 1 protein (DDX1) may play a role in the progression of neuroblastoma (Nb) and retinoblastoma (Rb) tumors (Godbout, R. et al. (1998) J. Biol. Chem. 273:21161-21168). These observations suggest that DDX1 may promote or enhance tumor progression by altering the normal secondary structure and expression levels of RNA in cancer cells. Other DEAD-box helicases have been implicated either directly or indirectly in tumorigenesis. (Discussed in Godbout, supra.) For example, murine p68 is mutated in ultraviolet light-induced tumors, and human DDX6 is located at a chromosomal breakpoint associated with B-cell lymphoma. Similarly, a chimeric protein comprised of DDX10 and NUP98, a nucleoporin protein, may be involved in the pathogenesis of certain myeloid malignancies.

Topoisomerases

Besides the need to separate DNA strands prior to replication, the two strands must be “unwound” from one another prior to their separation by DNA helicases. This function is performed by proteins known as DNA topoisomerases. DNA topoisomerase effectively acts as a reversible nuclease that hydrolyzes a phosphodiesterase bond in a DNA strand, permits the two strands to rotate freely about one another to remove the strain of the helix, and then rejoins the original phosphodiester bond between the two strands. Topoisomerases are essential enzymes responsible for the topological rearrangement of DNA brought about by transcription, replication, chromatin formation, recombination, and chromosome segregation. Superhelical coils are introduced into DNA by the passage of processive enzymes such as RNA polymerase, or by the separation of DNA strands by a helicase prior to replication. Knotting and concatenation can occur in the process of DNA synthesis, storage, and repair. All topoisomerases work by breaking a phosphodiester bond in the ribose-phosphate backbone of DNA. A catalytic tyrosine residue on the enzyme makes a nucleophilic attack on the scissile phosphodiester bond, resulting in a reaction intermediate in which a covalent bond is formed between the enzyme and one end of the broken strand. A tyrosine-DNA phosphodiesterase functions in DNA repair by hydrolyzing this bond in occasional dead-end topoisomerase I-DNA intermediates (Pouliot, J. J. et al. (1999) Science 286:552-555).

Two types of DNA topoisomerase exist, types I and II. Type I topoisomerases work as monomers, making a break in a single strand of DNA while type II topoisomerases, working as homodimers, cleave both strands. DNA Topoisomerase I causes a single-strand break in a DNA helix to allow the rotation of the two strands of the helix about the remaining phosphodiester bond in the opposite strand. DNA topoisomerase I[ causes a transient break in both strands of a DNA helix where two double helices cross over one another. This type of topoisomerase can efficiently separate two interlocked DNA circles (Alberts, supra, pp.260-262). Type II topoisomerases are largely confined to proliferating cells in eukaryotes, such as cancer cells. For this reason they are targets for anticancer drugs. Topoisomerase II has been implicated in multi-drug resistance (MDR) as it appears to aid in the repair of DNA damage inflicted by DNA binding agents such as doxorubicin and vincristine.

The topoisomerase I family includes topoisomerases I and III (topo I and topo III). The crystal structure of human topoisomerase I suggests that rotation about the intact DNA strand is partially controlled by the enzyme. In this “controlled rotation” model, protein-DNA interactions limit the rotation, which is driven by torsional strain in the DNA (Stewart, L. et al. (1998) Science 379:1534-1541). Structurally, topo I can be recognized by its catalytic tyrosine residue and a number of other conserved residues in the active site region. Topo I is thought to function during transcription. Two topo IIIs are known in humans, and they are homologous to prokaryotic topoisomerase I, with a conserved tyrosine and active site signature specific to this family. Topo III has been suggested to play a role in meiotic recombination. A mouse topo III is highly expressed in testis tissue and its expression increases with the increase in the number of cells in pachytene (Seki, T. et al. (1998) J. Biol. Chem. 273:28553-28556).

The topoisomerase II family includes two isozymes (IIα and IIβ) encoded by different genes. Topo II cleaves double stranded DNA in a reproducible, nonrandom fashion, preferentially in an AT rich region, but the basis of cleavage site selectivity is not known. Structurally, topo II is made up of four domains, the first two of which are structurally similar and probably distantly homologous to similar domains in eukaryotic topo I. The second domain bears the catalytic tyrosine, as well as a highly conserved pentapeptide. The Ha isoform appears to be responsible for unlinkig DNA during chromosome segregation. Cell lines expressing IIα but not IIβ suggest that IIIβ is dispensable in cellular processes; however, IIβ knockout mice died perinatally due to a failure in neural development. That the major abnormalities occurred in predominandy late developmental events (neurogenesis) suggests that IIβ is needed not at mitosis, but rather during DNA repair (Yang, X. et al. (2000) Science 287:131-134).

Topoisomerases have been implicated in a number of disease states, and topoisomerase poisons have proven to be effective anti-tumor drugs for some human malignancies. Topo I is mislocalized in Fanconi's anemia, and may be involved in the chromosomal breakage seen in this disorder (Wunder, E. (1984) Hum. Genet. 68:276-281). Overexpression of a truncated topo III in ataxia-telangiectasia (A-T) cells partially suppresses the A-T phenotype, probably through a dominant negative mechanism. This suggests that topo III is deregulated in A-T (Fritz, E. et al. (1997) Proc. Natl. Acad. Sci. USA 94:4538-4542). Topo III also interacts with the Bloom's Syndrome gene product, and has been suggested to have a role as a tumor suppressor (Wu, L. et al. (2000) J. Biol. Chem. 275:9636-9644). Aberrant topo II activity is often associated with cancer or increased cancer risk. Greatly lowered topo II activity has been found in some, but not all A-T cell lines (Mohamed, R. et al. (1987) Biochem. Biophys. Res. Commun. 149:233-238). On the other hand, topo II can break DNA in the region of the A-T gene (ATM), which controls all DNA damage-responsive cell cycle checkpoints (Kaufmann, W. K. (1998) Proc. Soc. Exp. Biol. Med. 217:327-334). The ability of topoisomerases to break DNA has been used as the basis of antitumor drugs. Topoisomerase poisons act by increasing the number of dead-end covalent DNA-enzyme complexes in the cell, ultimately triggering cell death pathways (Fortune, J. M. and N. Osheroff (2000) Prog. Nucleic Acid Res. Mol. Biol. 64:221-253; Guichard, S. M. and M. K. Danks (1999) Curr. Opin. Oncol. 11:482-489). Antibodies against topo I are found in the serum of systemic sclerosis patients, and the levels of the antibody may be used as a marker of pulmonary involvement in the disease (Diot, E. et al. (1999) Chest 116:715-720). Finally, the DNA binding region of human topo I has been used as a DNA delivery vehicle for gene therapy (Chen, T. Y. et al. (2000) Appl. Microbiol. Biotechnol 53:558-567).

Recombinases

Genetic recombination is the process of rearranging DNA sequences within an organism's genome to provide genetic variation for the organism in response to changes in the environment. DNA recombination allows variation in the particular combination of genes present in an individual's genome, as well as the timing and level of expression of these genes. (See Alberts, supra pp. 263-273.) Two broad classes of genetic recombination are commonly recognized, general recombination and site-specific recombination. General recombination involves genetic exchange between any homologous pair of DNA sequences usually located on two copies of the same chromosome. The process is aided by enzymes, recombinases, that “nick” one strand of a DNA duplex more or less randomly and permit exchange with a complementary strand on another duplex. The process does not normally change the arrangement of genes in a chromosome. In site-specific recombination, the recombinase recognizes specific nucleotide sequences present in one or both of the recombining molecules. Base-pairing is not involved in this form of recombination and therefore it does not require DNA homology between the recombining molecules. Unlike general recombination, this form of recombination can alter the relative positions of nucleotide sequences in chromosomes.

RNA Metabolism

Ribonucleic acid (RNA) is a linear single-stranded polymer of four nucleotides, ATP, CTP, UTP, and GTP. In most organisms, RNA is transcribed as a copy of deoxyribonucleic acid (DNA), the genetic material of the organism. In retroviruses RNA rather than DNA serves as the genetic material. RNA copies of the genetic material encode proteins or serve various structural, catalytic, or regulatory roles in organisms. RNA is classified according to its cellular localization and function. Messenger RNAs (mRNAs) encode polypeptides. Ribosomal RNAs (rRNAs) are assembled, along with ribosomal proteins, into ribosomes, which are cytoplasmic particles that translate mRNA into polypeptides. Transfer RNAs (tRNAs) are cytosolic adaptor molecules that function in mRNA translation by recognizing both an mRNA codon and the amino acid that matches that codon. Heterogeneous nuclear RNAs (hnRNAs) include mRNA precursors and other nuclear RNAs of various sizes. Small nuclear RNAs (snRNAs) are a part of the nuclear spliceosome complex that removes intervening, non-coding sequences (introns) and rejoins exons in pre-mRNAs.

Proteins are associated with RNA during its transcription from DNA, RNA processing, and translation of mRNA into protein. Proteins are also associated with RNA as it is used for structural, catalytic, and regulatory purposes.

RNA Processing

Ribosomal RNAs (rRNAs) are assembled, along with ribosomal proteins, into ribosomes, which are cytoplasmic particles that translate messenger RNA (mRNA) into polypeptides. The eukaryotic ribosome is composed of a 60S (large) subunit and a 40S (small) subunit, which together form the 80S ribosome. In addition to the 18S, 28S, 5S, and 5.8S rRNAs, ribosomes contain from 50 to over 80 different ribosomal proteins, depending on the organism. Ribosomal proteins are classified according to which subunit they belong (i.e., L, if associated with the large 60S large subunit or S if associated with the small 40S subunit). E. coli ribosomes have been the most thoroughly studied and contain 50 proteins, many of which are conserved in all life forms. The structures of nine ribosomal proteins have been solved to less than 3.0 D resolution (i.e., S5, S6, S17, L1, L6, L9, L12, L14, L30), revealing common motifs, such as b-a-b protein folds in addition to acidic and basic RNA-binding motifs positioned between b-strands. Most ribosomal proteins are believed to contact rRNA directly (reviewed in Liljas, A. and Garber, M. (1995) Curr. Opin. Struct. Biol. 5:721-727; see also Woodson, S. A. and Leontis, N. B. (1998) Curr. Opin. Struct. Biol. 8:294-300; Ramakrishnan, V. and White, S. W. (1998) Trends Biochem. Sci. 23:208-212).

Ribosomal proteins may undergo post-translational modifications or interact with other ribosome-associated proteins to regulate translation. For example, the highly homologous 40S ribosomal protein S6 kinases (S6K1 and S6K2) play a key role in the regulation of cell growth by controlling the biosynthesis of translational components which make up the protein synthetic apparatus (including the ribosomal proteins). In the case of S6K1, at least eight phosphorylation sites are believed to mediate kinase activation in a hierarchical fashion (Dufner and Thomas (1999) Exp. Cell. Res. 253:100-109). Some of the ribosomal proteins, including L1, also function as translational repressors by binding to polycistronic mRNAs encoding ribosomal proteins (reviewed in Liljas, supra and Garber, supra).

Recent evidence suggests that a number of ribosomal proteins have secondary functions independent of their involvement in protein biosynthesis. These proteins function as regulators of cell proliferation and, in some instances, as inducers of cell death. For example, the expression of human ribosomal protein L13a has been shown to induce apoptosis by arresting cell growth in the G2/M phase of the cell cycle. Inhibition of expression of L13a induces apoptosis in target cells, which suggests that this protein is necessary, in the appropriate amount, for cell survival. Similar results have been obtained in yeast where inactivation of yeast homologues of L13a, rp22 and rp23, results in severe growth retardation and death. A closely related ribosomal protein, L7, arrests cells in G1 and also induces apoptosis. Thus, it appears that a subset of ribosomal proteins may function as cell cycle checkpoints and compose a new family of cell proliferation regulators.

Mapping of individual ribosomal proteins on the surface of intact ribosomes is accomplished using 3D immunocryoelectronmicroscopy, whereby antibodies raised against specific ribosomal proteins are visualized. Progress has been made toward the mapping of L1, L7, and L12 while the structure of the intact ribosome has been solved to only 20-25D resolution and inconsistencies exist among different crude structures (Frank, J. (1997) Curr. Opin. Struct. Biol. 7:266-272).

Three distinct sites have been identified on the ribosome. The aminoacyl-tRNA acceptor site (A site) receives charged tRNAs (with the exception of the initiator-tRNA). The peptidyl-tRNA site (P site) binds the nascent polypeptide as the amino acid from the A site is added to the elongating chain. Deacylated tRNAs bind in the exit site (E site) prior to their release from the ribosome. The structure of the ribosome is reviewed in Stryer, L. (1995) Biochemistr, W. H. Freeman and Company, New York N.Y., pp. 888-9081; Lodish, H. et al. (1995) Molecular Cell Biology, Scientific American Books, New York N.Y., pp. 119-138; and Lewin, B (1997) Genes VI, Oxford University Press, Inc. New York, N.Y.).

Various proteins are necessary for processing of transcribed RNAs in the nucleus. Pre-mRNA processing steps include capping at the 5′ end with methylguanosine, polyadenylating the 3′ end, and splicing to remove introns. The primary RNA transcript from DNA is a faithful copy of the gene containing both exon and intron sequences, and the latter sequences must be cut out of the RNA transcript to produce a mRNA that codes for a protein. This “splicing” of the mRNA sequence takes place in the nucleus with the aid of a large, multicomponent ribonucleoprotein complex known as a spliceosome. The spliceosomal complex is comprised of five small nuclear ribonucleoprotein particles (snRNPs) designated U1, U2, U4, U5, and U6. Each snRNP contains a single species of snRNA and about ten proteins. The RNA components of some snRNPs recognize and base-pair with intron consensus sequences. The protein components mediate spliceosome assembly and the splicing reaction. Autoantibodies to snRNP proteins are found in the blood of patients with systemic lupus erythematosus (Stryer, L. (1995) Biochemistry, W. H. Freeman and Company, New York N.Y., p. 863).

Heterogeneous nuclear ribonucleoproteins nRNPs) have been identified that have roles in splicing, exporting of the mature RNAs to the cytoplasm, and mRNA translation (Biamonti, G. et al. (1998) Clin. Exp. Rheumatol. 16:317-326). Some examples of hnRNPs include the yeast proteins Hrp1p, involved in cleavage and polyadenylation at the 3′ end of the RNA; Cbp80p, involved in capping the 5′ end of the RNA; and Np13p, a homolog of mammalian hnRNP A1, involved in export of mRNA from the nucleus (Shen, E. C. et al. (1998) Genes Dev. 12:679-691). HnRNPs have been shown to be important targets of the autoimmune response in rheumatic diseases (Biamonti, supra).

Many snRNP and hnRNP proteins are characterized by an RNA recognition motif (RRM). (Reviewed in Birney, E. et al. (1993) Nucleic Acids Res. 21:5803-5816.) The RRM is about 80 amino acids in length and forms four β-strands and two α-helices arranged in an α/β sandwich. The RRM contains a core RNP-1 octapeptide motif along with surrounding conserved sequences. In addition to snRNP proteins, examples of RNA-binding proteins which contain the above motifs include heteronuclear ribonucleoproteins which stabilize nascent RNA and factors which regulate alternative splicing. Alternative splicing factors include developmentally regulated proteins, specific examples of which have been identified in lower eukaryotes such as Drosophila melanogaster and Caenorhabditis elegans. These proteins play key roles in developmental processes such as pattern formation and sex determination, respectively. (See, for example, Hodgkin, J. et al. (1994) Development 120:3681-3689.)

The 3′ ends of most eukaryote mRNAs are also posttranscriptionally modified by polyadenylation. Polyadenylation proceeds through two enzymatically distinct steps: (i) the endonucleolytic cleavage of nascent mRNAs at cis-acting polyadenylation signals in the 3′-untranslated (non-coding) region and (ii) the addition of a poly(A) tract to the 5′ mRNA fragment. The presence of cis-acting RNA sequences is necessary for both steps. These sequences include 5′-AAUAAA-3′ located 10-30 nucleotides upstream of the cleavage site and a less well-conserved GU- or U-rich sequence element located 10-30 nucleotides downstream of the cleavage site. Cleavage stimulation factor (CstF), cleavage factor I (CF I), and cleavage factor II (CF II) are involved in the cleavage reaction while cleavage and polyadenylation specificity factor (CPSF) and poly(A) polymerase (PAP) are necessary for both cleavage and polyadenylation. An additional enzyme, poly(A)-binding protein II (PAB II), promotes poly(A) tract elongation (Rüegsegger, U. et al. (1996) J. Biol. Chem. 271:6107-6113; and references within).

Translation

Correct translation of the genetic code depends upon each amino acid forming a linkage with the appropriate transfer RNA (tRNA). The aminoacyl-tRNA synthetases (aaRSs) are essential proteins found in all living organisms. The aaRSs are responsible for the activation and correct attachment of an amino acid with its cognate tRNA, as the first step in protein biosynthesis. Prokaryotic organisms have at least twenty different types of aaRSs, one for each different amino acid, while eukaryotes usually have two aaRSs, a cytosolic form and a mitochondrial form, for each different amino acid. The 20 aaRS enzymes can be divided into two structural classes. Class I enzymes add amino acids to the 2′ hydroxyl at the 3′ end of tRNAs while Class II enzymes add amino acids to the 3′ hydroxyl at the 3′ end of tRNAs. Each class is characterized by a distinctive topology of the catalytic domain. Class I enzymes contain a catalytic domain based on the nucleotide-binding Rossman ‘fold’. In particular, a consensus tetrapeptide motif is highly conserved (Prosite Document PDOC00161, Aminoacyl-transfer RNA synthetases class-I signature). Class I enzymes are specific for arginine, cysteine, glutarnic acid, glutamine, isoleucine, leucine, methionine, tyrosine, tryptophan, and valine. Class II enzymes contain a central catalytic domain, which consists of a seven-stranded antiparallel β-sheet domain, as well as N- and C-terminal regulatory domains. Class II enzymes are separated into two groups based on the heterodimeric or homodimeric structure of the enzyme; the latter group is further subdivided by the structure of the N- and C-terminal regulatory domains (Hartlein, M. and Cusack, S. (1995) J. Mol. Evol. 40:519-530). Class II enzymes are specific for alanine, asparagine, aspartic acid, glycine, histidine, lysine, phenylalanine, proline, serine, and threonine.

Certain aaRSs also have editing functions. IleRS, for example, can misactivate valine to form Val-tRNA^{Ile}, but this product is cleared by a hydrolytic activity that destroys the mischarged product. This editing activity is located within a second catalytic site found in the connective polypeptide 1 region (CP1), a long insertion sequence within the Rossman fold domain of Class I enzymes (Schimmel, P. et al. (1998) FASEB J. 12:1599-1609). AaRSs also play a role in tRNA processing. It has been shown that mature tRNAs are charged with their respective amino acids in the nucleus before export to the cytoplasm, and charging may serve as a quality control mechanism to insure the tRNAs are functional (Martinis, S. A. et al. (1999) EMBO J. 18:4591-4596).

Under optimal conditions, polypeptide synthesis proceeds at a rate of approximately 40 amino acid residues per second. The rate of misincorporation during translation in on the order of 10⁻⁴ and is primarily the result of aminoacyl-t-RNAs being charged with the incorrect amino acid. Incorrectly charged tRNA are toxic to cells as they result in the incorporation of incorrect amino acid residues into an elongating polypeptide. The rate of translation is presumed to be a compromise between the optimal rate of elongation and the need for translational fidelity. Mathematical calculations predict that 10⁻⁴ is indeed the maximum acceptable error rate for protein synthesis in a biological system (reviewed in Stryer, supra; and Watson, J. et al. (1987) The Benjamin/Cummings Publishing Co., Inc. Menlo Park, Calif.). A particularly error prone aminoacyl-tRNA charging event is the charging of tRNA^{Gln}, with Gln. A mechanism exits for the correction of this mischarging event which likely has its origins in evolution. Gin was among the last of the 20 naturally occurring amino acids used in polypeptide synthesis to appear in nature. Gram positive eubacteria, cyanobacteria, Archeae, and eukaryotic organelles possess a noncanonical pathway for the synthesis of Gln-tRNA^{Gin} based on the transformation of Glu-tRNA^{Gln} (synthesized by Glu-tRNA synthetase, GluRS) using the enzyme Glu-tRNA^{Gin} amidotransferase (Glu-AdT). The reactions involved in the transamidation pathway are as follows (Curnow, A. W. et al. (1997) Nucleic Acids Symposium 36:2-4):

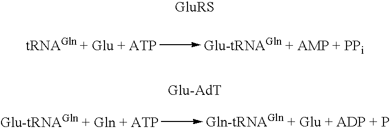

A similar enzyme, Asp-tRNA^{Asn} amidotransferase, exists in Archaea, which transforms Asp-tRNA^{Asn} to Asn-tRNA^{Asn}. Formylase, the enzyme that transforms Met-tRNA^{fMet} to fMet-tRNA^{fMet} in eubacteria, is likely to be a related enzyme. A hydrolytic activity has also been identified that destroys mischarged Val-tRNA^{Ile} (Schimmel, P. et al. (1998) FASEB J. 12:1599-1609). One likely scenario for the evolution of Glu-AdT in primitive life forms is the absence of a specific glutaminyl-tRNA synthetase (GlnRS), requiring an alternative pathway for the synthesis of Gln-tRNA^{Gln}. In fact, deletion of the Glu-AdT operon in Gram positive bacteria is lethal (Curnow, A. W. et al. (1997) Proc. Natl. Acad. Sci. USA 94:11819-11826). The existence of GluRS activity in other organisms has been inferred by the high degree of conservation in translation machinery in nature; however, GluRS has not been identified in all organisms, including Homo sapiens. Such an enzyme would be responsible for ensuring translational fidelity and reducing the synthesis of defective polypeptides.

In addition to their function in protein synthesis, specific aminoacyl tRNA synthetases also play roles in cellular fidelity, RNA splicing, RNA trafficking, apoptosis, and transcriptional and translational regulation. For example, human tyrosyl-tRNA synthetase can be proteolytically cleaved into two fragments with distinct cytokine activities. The carboxy-terminal domain exhibits monocyte and leukocyte chemotaxis activity as well as stimulating production of myeloperoxidase, tumor necrosis factor-α, and tissue factor. The N-terminal domain binds to the interleukin-8 type A receptor and functions as an interleukin-8-like cytokine. Human tyrosyl-tRNA synthetase is secreted from apoptotic tumor cells and may accelerate apoptosis (Wakasugi, K., and Schimmel, P. (1999) Science 284:147-151). Mitochondrial Neurospora crassa TyrRS and S. cerevisiae LeuRS are essential factors for certain group I intron splicing activities, and human mitochondrial LeuRS can substitute for the yeast LeuRS in a yeast null strain. Certain bacterial aaRSs are involved in regulating their own transcription or translation (Martinis, supra). Several aaRSs are able to synthesize diadenosine oligophosphates, a class of signalling molecules with roles in cell proliferation, differentiation, and apoptosis (Kisselev, L. L et al. (1998) FEBS Lett. 427:157-163; Vartanian, A. et al. (1999) FEBS Lett. 456:175-180).

Autoantibodies against aminoacyl-tRNAs are generated by patients with autoimmune diseases such as rheumatic arthritis, dermatomyositis and polymyositis, and correlate strongly with complicating interstitial lung disease (ILD) (Freist, W. et al. (1999) Biol. Chem. 380:623-646; Freist, W. et al. (1996) Biol. Chem. Hoppe Seyler 377:343-356). These antibodies appear to be generated in response to viral infection, and coxsackie virus has been used to induce experimental viral myositis in animals.

Comparison of aaRS structures between humans and pathogens has been useful in the design of novel antibiotics (Schimmel, supra). Genetically engineered aaRSs have been utilized to allow site-specific incorporation of unnatural amino acids into proteins in vivo (Liu, D. R. et al. (1997) Proc. Natl. Acad. Sci. USA 94:10092-10097).

tRNA Modifications

The modified ribonucleoside, pseudouridine (ψ), is present ubiquitously in the anticodon regions of transfer RNAs (tRNAs), large and small ribosomal RNAs (rRNAs), and small nuclear RNAs (snRNAs). y is the most common of the modified nucleosides (i.e., other than G, A, U, and C) present in tRNAs. Only a few yeast tRNAs that are not involved in protein synthesis do not contain ψ (Cortese, R. et al. (1974) J. Biol. Chem. 249:1103-1108). The enzyme responsible for the conversion of uridine to ψ, pseudouridine synthase (pseudouridylate synthase), was first isolated from Salmonella typhimurium (Arena, F. et al. (1978) Nucleic Acids Res. 5:4523-4536). The enzyme has since been isolated from a number of mammals, including steer and mice (Green, C. J. et al. (1982) J. Biol. Chem. 257:3045-52; and Chen, J. and Patton, J. R. (1999) RNA 5:409-419). tRNA pseudouridine synthases have been the most extensively studied members of the family. They require a thiol donor (e.g., cysteine) and a monovalent cation (e.g., ammonia or potassium) for optimal activity. Additional cofactors or high energy molecules (e.g., ATP or GTP) are not required (Green, supra). Other eukaryotic pseudouridine synthases have been identified that appear to be specific for rRNA (reviewed in Smith, C. M. and Steitz, J. A. (1997) Cell 89:669-672) and a dual-specificity enzyme has been identified that uses both tRNA and rRNA substrates (Wrzesinski, J. et al. (1995) RNA 1: 437-448). The absence of ψ in the anticodon loop of tRNAs results in reduced growth in both bacteria (Singer, C. E. et al. (1972) Nature New Biol. 238:72-74) and yeast (Lecointe, F. (1998) J. Biol. Chem. 273:1316-1323), although the genetic defect is not lethal.

Another ribonucleoside modification that occurs primarily in eukaryotic cells is the conversion of guanosine to N²,N²-dimethylguanosine (m²₂G) at position 26 or 10 at the base of the D-stem of cytosolic and mitochondrial tRNAs. This posttranscriptional modification is believed to stabilize tRNA structure by preventing the formation of alternative tRNA secondary and tertiary structures. Yeast tRNA^{Asp} is unusual in that it does not contain this modification. The modification does not occur in eubacteria, presumably because the structure of tRNAs in these cells and organelles is sequence constrained and does not require posttranscriptional modification to prevent the formation of alternative structures (Steinberg, S. and Cedergren, R. (1995) RNA 1:886-891, and references within). The enzyme responsible for the conversion of guanosine to m²₂G is a 63 kDa S-adenosylmethionine (SAM)-dependent tRNA N²,N²-dimethyl-guanosine methyltransferase (also referred to as the TRM1 gene product and herein referred to as TRM) (Edqvist, J. (1995) Biochimie 77:54-61). The enzyme localizes to both the nucleus and the mitochondria (Li, J-M. et al. (1989) J. Cell Biol. 109:1411-1419). Based on studies with TRM from Xenopus laevis, there appears to be a requirement for base pairing at positions C11-G24 and G10-C25 immediately preceding the G26 to be modified, with other structural features of the tRNA also being required for the proper presentation of the G26 substrate (Edqvist. J. et al. (1992) Nucleic Acids Res. 20:6575-6581). Studies in yeast suggest that cells carrying a weak ochre tRNA suppressor (sup3-i) are unable to suppress translation termination in the absence of TRM activity, suggesting a role for TRM in modifying the frequency of suppression in eukaryotic cells (Niederberger, C. et al. (1999) FEBS Lett. 464:67-70), in addition to the more general function of ensuring the proper three-dimensional structures for tRNA.

Translation Initiation

Initiation of translation can be divided into three stages. The first stage brings an initiator transfer RNA (Met-tRNA_{f}) together with the 40S ribosomal subunit to form the 43S preinitiation complex. The second stage binds the 43S preinitiation complex to the mRNA, followed by migration of the complex to the correct AUG initiation codon. The third stage brings the 60S ribosomal subunit to the 40S subunit to generate an 80S ribosome at the inititation codon. Regulation of translation primarily involves the first and second stage in the initiation process (V. M. Pain (1996) Eur. J. Biochem. 236:747-771).

Several initiation factors, many of which contain multiple subunits, are involved in bringing an initiator tRNA and the 40S ribosomal subunit together. eIF2, a guanine nucleotide binding protein, recruits the initiator tRNA to the 40S ribosomal subunit. Only when eIF2 is bound to GTP does it associate with the initiator tRNA. eIF2B, a guanine nucleotide exchange protein, is responsible for converting eIF2 from the GDP-bound inactive form to the GTP-bound active form. Two other factors, eIFIA and eIF3 bind and stabilize the 40S subunit by interacting with the 18S ribosomal RNA and specific ribosomal structural proteins. eIF3 is also involved in association of the 40S ribosomal subunit with mRNA. The Met-tRNA_{f}, eIF1A, eIF3, and 40S ribosomal subunit together make up the 43S preinitiation complex (Pain, supra).

eIF2 plays a central role in the maintenance of a rate-limiting step in mRNA translation. In this step, eIF2 binds GTP and Met-tRNAi and transfers Met-tRNAi to the 40S ribosomal subunit. At the end of the initiation process, GTP bound to eIF2 is hydrolyzed to GDP and the eIF2.GDP complex is released from the ribosome. The exchange of GDP bound to eIF2 for GTP is a prerequisite to binding Met-tRNAi and is mediated by a second initiation factor, eIF2B, a guanine nucleotide-exchange factor. Phosphorylation of eIF2 on its alpha-subunit converts eIF2 from a substrate of eIF2B into a competitive inhibitor. Thus, phosphorylation of eIF2 alpha effectively prevents formation of the e[F2.GTP.Met-tRNAi complex and inhibits global protein synthesis. Phosphorylation of eIF2 alpha occurs under a variety of conditions including viral infection, apoptosis, nutrient deprivation, heme-deprivation, and certain stresses. The 5′-untranslated region of hepatitis C virus (HCV) functions as an internal ribosome entry site (IRES) to initiate translation of HCV proteins. eIF2Bgamma and eIF2gamma are cellular factors involved in HCV IRES-mediated translation (Kimball, S. R. (1999) Int. J. Biochem. Cell Biol. 31:25-29; Webb, B. L. and Proud, C. G. (1997) Int. J. Biochem. Cell Biol. 29:1127-1131; Kruger M. et al. (2000) Proc. Natl. Acad. Sci. U S A 97:8566-8571).

Additional factors are required for binding of the 43S preinitiation complex to an mRNA molecule, and the process is regulated at several levels. eIF4F is a complex consisting of three proteins: eIF4E, eIF4A, and eIF4G. eIF4E recognizes and binds to the mRNA 5′-terminal m⁷GTP cap, eIF4A is a bidirectional RNA-dependent helicase, and eIF4G is a scaffolding polypeptide. eIF4G has three binding domains. The N-terminal third of eIF4G interacts with eIF4E, the central third interacts with eIF4A, and the C-terminal third interacts with eIF3 bound to the 43S preinitiation complex. Thus, eIF4G acts as a bridge between the 40S ribosomal subunit and the mRNA (M. W. Hentze (1997) Science 275:500-501).

The ability of eIF4F to initiate binding of the 43S preinitiation complex is regulated by structural features of the mRNA. The mRNA molecule has an untranslated region (UTR) between the 5′ cap and the AUG start codon. In some mRNAs this region forms secondary structures that impede binding of the 43S preinitiation complex. The helicase activity of eIF4A is thought to function in removing this secondary structure to facilitate binding of the 43S preinitiation complex (Pain, supra).

Translation Elongation

Elongation is the process whereby additional amino acids are joined to the initiator methionine to form the complete polypeptide chain. The elongation factors EF1 α, EF2 β γ, and EF2 are involved in elongating the polypeptide chain following initiation. EF1 α is a GTP-binding protein. In EF1 α's GTP-bound form, it brings an aminoacyl-tRNA to the ribosome's A site. The amino acid attached to the newly arrived aminoacyl-tRNA forms a peptide bond with the initiator methionine. The GTP on EF1 α is hydrolyzed to GDP, and EF1 α-GDP dissociates from the ribosome. EF1 β γ binds EF1 α-GDP and induces the dissociation of GDP from EF1 α allowing EF1 α to bind GTP and a new cycle to begin.

As subsequent aminoacyl-tRNAs are brought to the ribosome, EF-G, another GTP-binding protein, catalyzes the translocation of tRNAs from the A site to the P site and finally to the E site of the ribosome. This allows the ribosome and the mRNA to remain attached during translation.

The MCM domain is found in DNA-dependent ATPases required for the initiation of eukaryotic DNA replication. In eukaryotes there is a family of six proteins that contain this domain, MCM2 to MCM7 (Hu, B. et al. (1993) Nucleic Acids Res. 21:5289-5293).

Translation Termination

The release factor eRF carries out termination of translation. eRF recognizes stop codons in the mRNA, leading to the release of the polypeptide chain from the ribosome.

The apical ectodermal ridge (AER) is an essential structure for vertebrate limb development. Wnt3a is expressed during the induction of chick AER. Misexpression of Wnt3a induces ectopic expression of AER-specific genes in the limb ectoderm. The genes beta-catenin and Lef1 mimic the effect of Wnt3a. Blocking the intrinsic Lef1 activity disrupts AER formation. Hence, Wnt3a functions in AER formation through the beta-catenin/LEF1 pathway. In contrast, neither beta-catenin nor Lef1 affects the Wnt7a-regulated dorsoventral polarity of the limb. Thus, two related Wnt genes elicit distinct responses in the same tissues by using different intracellular pathways (Kengaku, M. et al.(1998) Science 280:1274-1277).

Treacher Collins Syndrome (TCS) is the most common of the human mandibulofacial dysostosis disorders. It shows autosomal dominant inheritance and occurs in 1 of 50,000 live births, with approximately 60% arising from new mutations. TCS symptoms show wide variability. The disease is deduced to be a result of interference in the development of the first and second branchial arches. The TCS gene, TCOF1, is localized to chromosome 5q31-33.3. There are ten identified mutations in TCOF1 consisting of nonsense mutations, insertions, deletions, or splicing mutations that apparently lead to premature termination of translation. Moreover, all are unique to each human family. TCOF1 encodes a low complexity protein of 1,411 amino acids, with repeated motifs that mirror the organization of its exons. These motifs are shared with nucleolar trafficking proteins in other species and are highly phosphorylated by casein kinase. The full-length TCOF1 protein sequence also contains nuclear and nucleolar localization signals and several polymorphisms. This data suggests that TCS results from defects in a nucleolar trafficking protein that is critically required during human craniofacial development (Wise, C. A. et al. (1997) Proc. Natl. Acad. Sci. U.S.A. 94:3110-3115).

Breast Cancer

There are more than 180,000 new cases of breast cancer diagnosed each year, and the mortality rate for breast cancer approaches 10% of all deaths in females between the ages of 45-54 (Gish, K. (1999) AWIS Magazine 28:7-10). However the survival rate based on early diagnosis of localized breast cancer is extremely high (97%), compared with the advanced stage of the disease in which the tumor has spread beyond the breast (22%). Current procedures for clinical breast examination are lacking in sensitivity and specificity, and efforts are underway to develop comprehensive gene expression profiles for breast cancer that may be used in conjunction with conventional screening methods to improve diagnosis and prognosis of this disease (Perou, C. M. et al. (2000) Nature 406:747-752).

Mutations in two genes, BRCA1 and BRCA2, are known to greatly predispose a woman to breast cancer and may be passed on from parents to children (Gish, supra). However, this type of hereditary breast cancer accounts for only about 5% to 9% of breast cancers, while the vast majority of breast cancer is due to non-inherited mutations that occur in breast epithelial cells.

The relationship between expression of epidermal growth factor (EGF) and its receptor, EGFR, to human mammary carcinoma has been particularly well studied. (See Khazaie, K. et al. (1993) Cancer and Metastasis Rev. 12:255-274, and references cited therein for a review of this area.) Overexpression of EGFR, particularly coupled with down-regulation of the estrogen receptor, is a marker of poor prognosis in breast cancer patients. In addition, EGFR expression in breast tumor metastases is frequently elevated relative to the primary tumor, suggesting that EGFR is involved in tumor progression and metastasis. This is supported by accumulating evidence that EGF has effects on cell functions related to metastatic potential, such as cell motility, chemotaxis, secretion and differentiation. Changes in expression of other members of the erbB receptor family, of which EGFR is one, have also been implicated in breast cancer. The abundance of erbB receptors, such as HER-2/neu, HER-3, and HER-4, and their ligands in breast cancer points to their functional importance in the pathogenesis of the disease, and may therefore provide targets for therapy of the disease (Bacus, S. S. et al. (1994) Am. J. Clin. Pathol. 102:S13-S24). Other known markers of breast cancer include a human secreted frizzled protein mRNA that is downregulated in breast tumors; the matrix G1a protein which is overexpressed is human breast carcinoma cells; Drg1 or RTP, a gene whose expression is diminished in colon, breast, and prostate tumors; maspin, a tumor suppressor gene downregulated in invasive breast carcinomas; and CaN19, a member of the S100 protein family, all of which are down regulated in mammary carcinoma cells relative to normal mammary epithelial cells (Zhou, Z. et al. (1998) Int. J. Cancer 78:95-99; Chen, L. et al. (1990) Oncogene 5:1391-1395; Ulrix, W. et al (1999) FEBS Lett 455:23-26; Sager, R. et al. (1996) Curr. Top. Microbiol. Immunol. 213:51-64; and Lee, S. W. et al. (1992) Proc. Natl. Acad. Sci. USA 89:2504-2508).

Cell lines derived from human mammary epithelial cells at various stages of breast cancer provide a useful model to study the process of malignant transformation and tumor progression as it has been shown that these cell lines retain many of the properties of their parental tumors for lengthy culture periods (Wistuba, I. I. et al. (1998) Clin. Cancer Res. 4:2931-2938). Such a model is particularly useful for comparing phenotypic and molecular characteristics of human mammary epithelial cells at various stages of malignant transformation.

Preadipocyte Cells

The most important function of adipose tissue is its ability to store and release fat during periods of feeding and fasting. White adipose tissue is the major energy reserve in periods of excess energy use, and its primary purpose is mobilization during energy deprivation. Understanding how the various molecules regulate adiposity and energy balance in physiological and pathophysiological situations may lead to the development of novel therapeutics for human obesity. Adipose tissue is also one of the important target tissues for insulin. Adipogenesis and insulin resistance in type II diabetes are linked and present intriguing relations. Most patients with type II diabetes are obese and obesity in turn causes insulin resistance.

The majority of research in adipocyte biology to date has been done using transformed mouse preadipocyte cell lines. The culture condition, which stimulates mouse preadipocyte differentiation is different from that for inducing human primary preadipocyte differentiation. In addition, primary cells are diploid and may therefore reflect the in vivo context better than aneuploid cell lines. Understanding the gene expression profile during adipogenesis in human will lead to understanding the fundamental mechanism of adiposity regulation. Furthermore, through comparing the gene expression profiles of adipogenesis between donor with normal weight and donor with obesity, identification of crucial genes, potential drug targets for obesity and type II diabetes, will be possible.

Peroxisome Proliferator-Activated Receptor Gamma Agonist

Thiazolidinediones (IZDs) act as agonists for the peroxisome-proliferator-activated receptor gamma (PPARγ), a member of the nuclear hormone receptor superfamily. TZDs reduce hyperglycemia, hyperinsulinemia, and hypertension, in part by promoting glucose metabolism and inhibiting gluconeogenesis. Roles for PPARγ and its agonists have been demonstrated in a wide range of pathological conditions including diabetes, obesity, hypertension, atherosclerosis, polycystic ovarian syndrome, and cancers such as breast, prostate, liposarcoma, and colon cancer.

The mechanism by which IZDs and other PPARγ agonists enhance insulin sensitivity is not fully understood, but may involve the ability of PPARγ to promote adipogenesis. When ectopically expressed in cultured preadipocytes, PPARγ is a potent inducer of adipocyte differentiation. IZDs, in combination with insulin and other factors, can also enhance differentiation of human preadipocytes in culture (Adams et al. (1997) J. Clin. Invest. 100:3149-3153). The relative potency of different mIDs in promoting adipogenesis in vitro is proportional to both their insulin sensitizing effects in vivo, and their ability to bind and activate PPARγ in vitro. Interestingly, adipocytes derived from omental adipose depots are refractory to the effects of TZDs. It has therefore been suggested that the insulin sensitizing effects of lZDs may result from their ability to promote adipogenesis in subcutaneous adipose depots (Adams et al., ibid). Further, dominant negative mutations in the PPARγ gene have been identified in two non-obese subjects with severe insulin resistance, hypertension, and overt non-insulin dependent diabetes mellitus (NIDDM) (Barroso et al. (1998) Nature 402:880-883).

NIDDM is the most common form of diabetes meffitus, a chronic metabolic disease that affects 143 million people worldwide. NIDDM is characterized by abnormal glucose and lipid metabolism that result from a combination of peripheral insulin resistance and defective insulin secretion. NIDDM has a complex, progressive etiology and a high degree of heritability. Numerous complications of diabetes including heart disease, stroke, renal failure, retinopathy, and peripheral neuropathy contribute to the high rate of morbidity and mortality.

At the molecular level, PPARγ functions as a ligand activated transcription factor. In the presence of ligand, PPARγ forms a heterodimer with the retinoid X receptor (RXR) which then activates transcription of target genes containing one or more copies of a PPARγ response element (PPRE). Many genes important in lipid storage and metabolism contain PPREs and have been identified as PPARγ targets, including PEPCK, aP2, LPL, ACS, and FAT-P (Auwerx, J. (1999) Diabetologia 42:1033-1049). Multiple ligands for PPARγ have been identified. These include a variety of fatty acid metabolites; synthetic drugs belonging to the TZD class, such as Pioglitazone and Rosiglitazone (BRLA9653); and certain non-glitazone tyrosine analogs such as G1262570 and GW1929. The prostaglandin derivative 15-dPGJ2 is a potent endogenous ligand for PPARγ.

Expression of PPARγ is very high in adipose but barely detectable in skeletal muscle, the primary site for insulin stimulated glucose disposal in the body. PPARγ is also moderately expressed in large intestine, kidney, liver, vascular smooth muscle, hematopoietic cells, and macrophages. The high expression of PPARγ in adipose suggests that the insulin sensitizing effects of TZDs may result from alterations in the expression of one or more PPARγ regulated genes in adipose tissue. Identification of PPARγ target genes will contribute to better drug design and the development of novel therapeutic strategies for diabetes, obesity, and other conditions.

Systematic attempts to identify PPARγ target genes have been made in several rodent models of obesity and diabetes (Suzuki et al. (2000) Jpn. J. Pharmacol. 84:113-123; Way et al. (2001) Endocrinology 142:1269-1277). However, a serious drawback of the rodent gene expression studies is that significant differences exist between human and rodent models of adipogenesis, diabetes, and obesity (Taylor (1999) Cell 97:9-12; Gregoire et al. (1998) Physiol. Reviews 78:783-809). Therefore, an unbiased approach to identifying TZD regulated genes in primary cultures of human tissues is necessary to fully elucidate the molecular basis for diseases associated with PPARγ activity.

Lung Cancer

Lung cancer is the leading cause of cancer death for men and the second leading cause of cancer death for women in the U.S. The vast majority of lung cancer cases are attributed to smoking tobacco, and increased use of tobacco products in third world countries is projected to lead to an epidemic of lung cancer in these countries. Exposure of the bronchial epithelium to tobacco smoke appears to result in changes in tissue morphology, which are thought to be precursors of cancer. Lung cancers are divided into four histopathologically distinct groups. Three groups (squamous cell carcinoma, adenocarcinoma, and large cell carcinoma) are classified as non-small cell lung cancers (NSCLCs). The fourth group of cancers is referred to as small cell lung cancer (SCLC). Collectively, NSCLCs account for ˜70% of cases while SCLCs account for ˜18% of cases. The molecular and cellular biology underlying the development and progression of lung cancer are incompletely understood.

Deletions on chromosome 3 are common in this disease and are thought to indicate the presence of a tumor suppressor gene in this region. Activating mutations in K-ras are commonly found in lung cancer and are the basis of one of the mouse models for the disease.

Colorectal Cancer

Colorectal cancer is the second leading cause of cancer deaths in the United States, and is thought to be a disease of aging since 90% of the total cases occur in individuals over the age of 55. A widely accepted hypothesis is that several mutations must accumulate over time in an individual who develops the disease. To understand the nature of gene alterations in colorectal cancer, a number of studies have focused on the inherited syndromes. The first, Familial Adenomatous Polyposis (FAP), is caused by mutations in the Adenomatous Polyposis Coli gene (APC), resulting in truncated or inactive forms of the protein. This tumor suppressor gene has been mapped to chromosome 5q. The second known inherited syndrome is hereditary nonpolyposis colorectal cancer (HNPCC), which is caused by mutations in mismatch repair genes. Although hereditary colon cancer syndromes occur in a small percentage of the population, and most colorectal cancers are considered sporadic, knowledge from studies of the hereditary syndromes can be applied broadly. For instance, somatic mutations in APC occur in at least 80% of sporadic colon tumors. APC mutations are thought to be the initiating event in disease progression. Other mutations occur subsequently. Approximately 50% of colorectal cancers contain activating mutations in ras, while 85% contain inactivating mutations in p53. Changes in all of these genes lead to gene expression changes in colon cancer.

Ovarian Cancer

Ovarian cancer is the leading cause of death from a gynecologic cancer. The majority of ovarian can-cers are derived from epithelial cells, and 70% of patients with epithelial ovarian cancers present with late-stage disease. Identification of early-stage markers for ovarian cancer would significantly increase the survival rate. Some of the molecular events implicated in ovarian cancer include mutation of p53 and niicrosatellite instability.

Additional Diseases and Related Factors

Tangier disease (TD) is a genetic disorder characterized by near absence of circulating HDL and the accumulation of cholesterol esters in many tissues, including tonsils, lymph nodes, liver, spleen, thymus, and intestine. Low levels of HDL represent a clear predictor of premature coronary artery disease and homozygous TD correlates with a four- to six-fold increase in cardiovascular disease compared to controls. The major cardioprotective activity of HDL is ascribed to its role in reverse cholesterol transport, the flux of cholesterol from peripheral cells such as tissue macrophages, through plasma lipoproteins to the liver. The HDL protein, apolipoprotein AI plays a major role in this process, interacting with the cell surface to remove excess cholesterol and phospholipids. This pathway is severely impaired in TD. The defect lies in a specific gene, the ABC1 transporter. This gene is a member of the family of ATP-binding cassette transporters, which utilize ATP hydrolysis to transport a variety of substrates across membranes.

The effects upon liver metabolism and hormone clearance mechanisms are important to understand the pharmacodynamics of a drug. The human C3A cell line is a clonal derivative of HepG2/C3 (hepatoma cell line, isolated from a 15-year-old male with liver tumor), which was selected for strong contact inhibition of growth. The use of a clonal population enhances the reproducibility of the cells. C3A cells have many characteristics of primary human hepatocytes in culture: i) expression of insulin receptor and insulin-like growth factor II receptor; ii) secretion of a high ratio of serum albumin compared with a-fetoprotein iii) conversion of ammonia to urea and glutamine;, iv) metabolism of aromatic amino acids; and v) proliferation in glucose-free and insulin-free medium. The C3A cell line is now well established as an in vitro model of the mature human liver (Mickelson et al. (1995) Hepatology 22:866-875; Nagendra et al. (1997) Am J Physiol 272:G408-G416).

Dexamethasone (DEX) is a synthetic glucocorticoid used as an anti-inflammatory or immuno-suppressive agent. Due to its greater ability to reach the central nervous system, DEX is usually the treatment of choice to control cerebral edema. Glucocorticoids are naturally occurring hormones that prevent or suppress inflammation and immune responses when administered at pharmacological doses. At the molecular level, unbound glucocorticoids readily cross cell membranes and bind with high affinity to specific cytoplasmic receptors. Subsequent to binding, transcription and protein synthesis are affected. The result can include inhibition of leukocyte infiltration at the site of inflammation, interference in the function of mediators of inflammatory response, and suppression of humoral immune responses. The anti-inflammatory actions of corticosteroids are thought to involve phospholipase A 2 inhibitory proteins, collectively called lipocortins. Lipocortins, in turn, control the biosynthesis of potent mediators of inflammation such as prostaglandins and leukotrienes by inliubiting the release of the precursor molecule arachidonic acid.

Human aortic endothelial cells (HMVECdNeos) are primary cells derived from the endothelium of the microvasculature of human skin. HMVECdNeos have been used as an experimental model for investigating in vitro the role of the endothelium in human vascular biology. Activation of the vascular endothelium is considered a central event in a wide range of both physiological and pathophysiological processes, such as vascular tone regulation, coagulation and thrombosis, atherosclerosis, and inflammation.

Tumor necrosis factor alpha (TNF-α) is a pleiotropic cytokine that plays a central role in mediation of the inflammatory response through activation of multiple signal transduction pathways. TNF-α is produced by activated lymphocytes, macrophages, and other white blood cells, and is known to activate endothelial cells. Monitoring the endothelial cell response to TNF-α at the level of mRNA expression can provide information necessary for better understanding of both TNF-α signaling and endothelial cell biology.

Dendritic cells (DCs), as antigen presenting cells, play a crucial role in the initiation of the immune response. DCs can be derived in vitro either from CD34+ bone marrow precursors (IDCs) or from peripheral blood monocytic cells (mDCs). In vivo, DCs reside in two distinct compartments: the peripheral tissues such as lung, skin, kidney, heart, and intestine; and in secondary lymphoid organs such as lymph node, spleen, and Peyer's patches. In the periphery, DCs are efficient antigen processing cells but are limited in their capacity to activate naive T cells. Upon activation (injury, inflammation, infection), DCs enter their final stage of maturation during which they downregulate the capacity to process new antigens, migrate out of the periphery into the secondary lymphoid organs, and acquire an extremely potent capacity to activate naive T cells. Factors such as cross linking the CD40 surface molecules or the presence of TNF-α can induce this final stage of maturation.

CD40 is a type I integral membrane glycoprotein belonging to the TNF-receptor family. It is expressed on all mature B lymphocytes, dendritic cells, and some epithelial cells. Antibodies specific for CD40 molecules can induce proliferation of B cells when presented with EL-4 or antibodies specific for CD20 molecules. Also, stimulation of B cells with anti-CD40 antibodies and IL-4 can induce the switch of immunoglobulin production to the IgE isotype.

Characterization of region-specific gene expression in the human brain provides a context and background for molecular neurobiology research in general. Information from RNA expression in these tissues may supply insight into the genetic basis of brain structure and function, which may in turn become useful in drug target discovery.

Array technology can provide a simple way to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for examining which genes are tissue specific, carrying out housekeeping functions, parts of a signaling cascade, or specifically related to a particular genetic predisposition, condition, disease, or disorder. The potential application of gene expression profiling is particularly relevant to improving diagnosis, prognosis, and treatment of disease. For example, both the levels and sequences expressed in tissues from subjects with diabetes may be compared with the levels and sequences expressed in normal tissue.

Expression Profiling

Microarrays are analytical tools used in bioanalysis. A microarray has a plurality of molecules spatially distributed over, and stably associated with, the surface of a solid support. Microarrays of polypeptides, polynucleotides, and/or antibodies have been developed and find use in a variety of applications, such as gene sequencing, monitoring gene expression, gene mapping, bacterial identification, drug discovery, and combinatorial chemistry.

One area in particular in which microarrays find use is in gene expression analysis. Array technology can provide a simple way to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for identifying genes that are tissue specific, are affected by a substance being tested in a toxicology assay, are part of a signaling cascade, carry out housekeeping functions, or are specifically related to a particular genetic predisposition, condition, disease, or disorder.

There is a need in the art for new compositions, including nucleic acids and proteins, for the diagnosis, prevention, and treatment of cell proliferative, neurological, developmental, and autoimmune/inflammatory disorders, and infections.

### SUMMARY OF THE INVENTION

Various embodiments of the invention provide purified polypeptides, nucleic acid-associated proteins, referred to collectively as ‘NAAP’ and individually as ‘NAAP-1,’ ‘NAAP-2,’ ‘NAAP-3,’ ‘NAAP-4,’ ‘NAAP-5,’ ‘NAAP-6,’ ‘NAAP-7,’ ‘NAAP-8,’ ‘NAAP-9,’ ‘NAAP10,’ ‘NAAP-11,’ ‘NAAP-12,’ ‘NAAP-13,’ ‘NAAP-14,’ ‘NAAP-15,’ ‘NAAP-16,’ ‘NAAP-17,’ ‘NAAP-18,’ ‘NAAP-19,’ ‘NAAP-20,’ ‘NAAP-21,’ ‘NAAP-22,’ ‘NAAP-23,’ ‘NAAP-24,’ ‘NAAP-25,’ ‘NAAP-26,’ ‘NAAP-27,’ ‘NAAP-28,’ ‘NAAP-29,’ ‘NAAP-30,’ ‘NAAP-31,’ ‘NAAP-32,’ NAAP-33,’ ‘NAAP-34,’ ‘NAAP-35,’ ‘NAAP-36,’ ‘NAAP-37,’ ‘NAAP-38,’ ‘NAAP-39,’ ‘NAAP-40,’ ‘NAAP-41,’ ‘NAAP-42,’ ‘NAAP-43,’ ‘NAAP-44,’ ‘NAAP-45,’ ‘NAAP-46,’ ‘NAAP-47,’ ‘NAAP-48,’ ‘NAAP-49,’ ‘NAAP-50,’ ‘NAAP-51,’ ‘NAAP-52,’ ‘NAAP-53,’ ‘NAAP-54,’ ‘NAAP-55,’ ‘NAAP-56,’ ‘NAAP-57,’ ‘and ‘NAAP-58’ and methods for using these proteins and their encoding polynucleotides for the detection, diagnosis, and treatment of diseases and medical conditions. Embodiments also provide methods for utilizing the purified nucleic acid-associated proteins and/or their encoding polynucleotides for facilitating the drug discovery process, including determination of efficacy, dosage, toxicity, and pharmacology. Related embodiments provide methods for utilizing the purified nucleic acid-associated proteins and/or their encoding polynucleotides for investigating the pathogenesis of diseases and medical conditions.

An embodiment provides an isolated polypeptide selected from the group consisting of a) a polypeptide comprising an amino acid sequence selected from the group consisting of SEQ ID NO:1-58, b) a polypeptide comprising a naturally occurring amino acid sequence at least 90% identical or at least about 90% identical to an amino acid sequence selected from the group consisting of SEQ ID NO:1-58, c) a biologically active fragment of a polypeptide having an amino acid sequence selected from the group consisting of SEQ ID NO:1-58, and d) an immunogenic fragment of a polypeptide having an amino acid sequence selected from the group consisting of SEQ ID NO:1-58. Another embodiment provides an isolated polypeptide comprising an amino acid sequence of SEQ ID NO:1-58.

Still another embodiment provides an isolated polynucleotide encoding a polypeptide selected from the group consisting of a) a polypeptide comprising an amino acid sequence selected from the group consisting of SEQ ID NO:1-58, b) a polypeptide comprising a naturally occurring amino acid sequence at least 90% identical or at least about 90% identical to an amino acid sequence selected from the group consisting of SEQ ID NO:1-58, c) a biologically active fragment of a polypeptide having an amino acid sequence selected from the group consisting of SEQ ID NO:1-58, and d) an immunogenic fragment of a polypeptide having an amino acid sequence selected from the group consisting of SEQ ID NO:1-58. In another embodiment, the polynucleotide encodes a polypeptide selected from the group consisting of SEQ ID NO:1-58. In an alternative embodiment, the polynucleotide is selected from the group consisting of SEQ ID NO:59-116.

Still another embodiment provides a recombinant polynucleotide comprising a promoter sequence operably linked to a polynucleotide encoding a polypeptide selected from the group consisting of a) a polypeptide comprising an amino acid sequence selected from the group consisting of SEQ ID NO:1-58, b) a polypeptide comprising a naturally occurring amino acid sequence at least 90% identical or at least about 90% identical to an amino acid sequence selected from the group consisting of SEQ ID NO:1-58, c) a biologically active fragment of a polypeptide having an amino acid sequence selected from the group consisting of SEQ ID NO:1-58, and d) an immunogenic fragment of a polypeptide having an amino acid sequence selected from the group consisting of SEQ ID NO:1-58. Another embodiment provides a cell transformed with the recombinant polynucleotide. Yet another embodiment provides a transgenic organism comprising the recombinant polynucleotide.

Another embodiment provides a method for producing a polypeptide selected from the group consisting of a) a polypeptide comprising an amino acid sequence selected from the group consisting of SEQ ID NO:1-58, b) a polypeptide comprising a naturally occurring amino acid sequence at least 90% identical or at least about 90% identical to an amino acid sequence selected from the group consisting of SEQ ID NO:1-58, c) a biologically active fragment of a polypeptide having an amino acid sequence selected from the group consisting of SEQ ID NO:1-58, and d) an immunogenic fragment of a polypeptide having an amino acid sequence selected from the group consisting of SEQ ID NO:1-58. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Yet another embodiment provides an isolated antibody which specifically binds to a polypeptide selected from the group consisting of a) a polypeptide comprising an amino acid sequence selected from the group consisting of SEQ ID NO:1-58, b) a polypeptide comprising a naturally occurring amino acid sequence at least 90% identical or at least about 90% identical to an amino acid sequence selected from the group consisting of SEQ ID NO:1-58, c) a biologically active fragment of a polypeptide having an amino acid sequence selected from the group consisting of SEQ ID NO:1-58, and d) an immunogenic fragment of a polypeptide having an amino acid sequence selected from the group consisting of SEQ ID NO:1-58.

Still yet another embodiment provides an isolated polynucleotide selected from the group consisting of a) a polynucleotide comprising a polynucleotide sequence selected from the group consisting of SEQ ID NO:59-116, b) a polynucleotide comprising a naturally occurring polynucleotide sequence at least 90% identical or at least about 90% identical to a polynucleotide sequence selected from the group consisting of SEQ ID NO:59-116, c) a polynucleotide complementary to the polynucleotide of a), d) a polynucleotide complementary to the polynucleotide of b), and e) an RNA equivalent of a)-d). In other embodiments, the polynucleotide can comprise at least about 20, 30, 40, 60, 80, or 100 contiguous nucleotides.

Yet another embodiment provides a method for detecting a target polynucleotide in a sample, said target polynucleotide being selected from the group consisting of a) a polynucleotide comprising a polynucleotide sequence selected from the group consisting of SEQ ID NO:59-116, b) a polynucleotide comprising a naturally occurring polynucleotide sequence at least 90% identical or at least about 90% identical to a polynucleotide sequence selected from the group consisting of SEQ ID NO:59-116, c) a polynucleotide complementary to the polynucleotide of a), d) a polynucleotide complementary to the polynucleotide of b), and e) an RNA equivalent of a)-d). The method comprises a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and b) detecting the presence or absence of said hybridization complex. In a related embodiment, the method can include detecting the amount of the hybridization complex. In still other embodiments, the probe can comprise at least about 20, 30, 40, 60, 80, or 100 contiguous nucleotides.

Still yet another embodiment provides a method for detecting a target polynucleotide in a sample, said target polynucleotide being selected from the group consisting of a) a polynucleotide comprising a polynucleotide sequence selected from the group consisting of SEQ ID NO:59-116, b) a polynucleotide comprising a naturally occurring polynucleotide sequence at least 90% identical or at least about 90% identical to a polynucleotide sequence selected from the group consisting of SEQ ID NO:59-116, c) a polynucleotide complementary to the polynucleotide of a), d) a polynucleotide complementary to the polynucleotide of b), and e) an RNA equivalent of a)-d). The method comprises a) amplifying said target polynucleotide or fragment thereof using polymerase chain reaction amplification, and b) detecting the presence or absence of said amplified target polynucleotide or fragment thereof. In a related embodiment, the method can include detecting the amount of the amplified target polynucleotide or fragment thereof.

Another embodiment provides a composition comprising an effective amount of a polypeptide selected from the group consisting of a) a polypeptide comprising an amino acid sequence selected from the group consisting of SEQ ID NO:1-58, b) a polypeptide comprising a naturally occurring amino acid sequence at least 90% identical or at least about 90% identical to an amino acid sequence selected from the group consisting of SEQ ID NO:1-58, c) a biologically active fragment of a polypeptide having an amino acid sequence selected from the group consisting of SEQ ID NO:1-58, and d) an immunogenic fragment of a polypeptide having an amino acid sequence selected from the group consisting of SEQ ID NO:1-58, and a pharmaceutically acceptable excipient. In one embodiment, the composition can comprise an amino acid sequence selected from the group consisting of SEQ ID NO:1-58. Other embodiments provide a method of treating a disease or condition associated with decreased or abnormal expression of functional NAAP, comprising administering to a patient in need of such treatment the composition.

Yet another embodiment provides a method for screening a compound for effectiveness as an agonist of a polypeptide selected from the group consisting of a) a polypeptide comprising an amino acid sequence selected from the group consisting of SEQ ID NO:1-58, b) a polypeptide comprising a naturally occurring amino acid sequence at least 90% identical or at least about 90% identical to an amino acid sequence selected from the group consisting of SEQ ID NO:1-58, c) a biologically active fragment of a polypeptide having an amino acid sequence selected from the group consisting of SEQ ID NO:1-58, and d) an immunogenic fragment of a polypeptide having an amino acid sequence selected from the group consisting of SEQ ID NO:1-58. The method comprises a) exposing a sample comprising the polypeptide to a compound, and b) detecting agonist activity in the sample. Another embodiment provides a composition comprising an agonist compound identified by the method and a pharmaceutically acceptable excipient. Yet another embodiment provides a method of treating a disease or condition associated with decreased expression of functional NAAP, comprising administering to a patient in need of such treatment the composition.

Still yet another embodiment provides a method for screening a compound for effectiveness as an antagonist of a polypeptide selected from the group consisting of a) a polypeptide comprising an amino acid sequence selected from the group consisting of SEQ ID NO:1-58, b) a polypeptide comprising a naturally occurring amino acid sequence at least 90% identical or at least about 90% identical to an amino acid sequence selected from the group consisting of SEQ ID NO:1-58, c) a biologically active fragment of a polypeptide having an amino acid sequence selected from the group consisting of SEQ ID NO:1-58, and d) an immunogenic fragment of a polypeptide having an amino acid sequence selected from the group consisting of SEQ ID NO:1-58. The method comprises a) exposing a sample comprising the polypeptide to a compound, and b) detecting antagonist activity in the sample. Another embodiment provides a composition comprising an antagonist compound identified by the method and a pharmaceutically acceptable excipient. Yet another embodiment provides a method of treating a disease or condition associated with overexpression of functional NAAP, comprising administering to a patient in need of such treatment the composition.

Another embodiment provides a method of screening for a compound that specifically binds to a polypeptide selected from the group consisting of a) a polypeptide comprising an amino acid sequence selected from the group consisting of SEQ ID NO:1-58, b) a polypeptide comprising a naturally occurring amino acid sequence at least 90% identical or at least about 90% identical to an amino acid sequence selected from the group consisting of SEQ ID NO:1-58, c) a biologically active fragment of a polypeptide having an amino acid sequence selected from the group consisting of SEQ ID NO:1-58, and d) an immunogenic fragment of a polypeptide having an amino acid sequence selected from the group consisting of SEQ ID NO:1-58. The method comprises a) combining the polypeptide with at least one test compound under suitable conditions, and b) detecting binding of the polypeptide to the test compound, thereby identifying a compound that specifically binds to the polypeptide.

Yet another embodiment provides a method of screening for a compound that modulates the activity of a polypeptide selected from the group consisting of a) a polypeptide comprising an amino acid sequence selected from the group consisting of SEQ ID NO:1-58, b) a polypeptide comprising a naturally occurring amino acid sequence at least 90% identical or at least about 90% identical to an amino acid sequence selected from the group consisting of SEQ ID NO:1-58, c) a biologically active fragment of a polypeptide having an amino acid sequence selected from the group consisting of SEQ ID NO:1-58, and d) an immunogenic fragment of a polypeptide having an amino acid sequence selected from the group consisting of SEQ ID NO:1-58. The method comprises a) combining the polypeptide with at least one test compound under conditions permissive for the activity of the polypeptide, b) assessing the activity of the polypeptide in the presence of the test compound, and c) comparing the activity of the polypeptide in the presence of the test compound with the activity of the polypeptide in the absence of the test compound, wherein a change in the activity of the polypeptide in the presence of the test compound is indicative of a compound that modulates the activity of the polypeptide.

Still yet another embodiment provides a method for screening a compound for effectiveness in altering expression of a target polynucleotide, wherein said target polynucleotide comprises a polynucleotide sequence selected from the group consisting of SEQ ID NO:59-116, the method comprising a) exposing a sample comprising the target polynucleotide to a compound, b) detecting altered expression of the target polynucleotide, and c) comparing the expression of the target polynucleotide in the presence of varying amounts of the compound and in the absence of the compound.

Another embodiment provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide selected from the group consisting of i) a polynucleotide comprising a polynucleotide sequence selected from the group consisting of SEQ ID NO:59-116, ii) a polynucleotide comprising a naturally occurring polynucleotide sequence at least 90% identical or at least about 90% identical to a polynucleotide sequence selected from the group consisting of SEQ ID NO:59-116, iii) a polynucleotide having a sequence complementary to i), iv) a polynucleotide complementary to the polynucleotide of ii), and v) an RNA equivalent of i)-iv). Hybridization occurs under conditions whereby a specific hybridization complex is formed between said probe and a target polynucleotide in the biological sample, said target polynucleotide selected from the group consisting of i) a polynucleotide comprising a polynucleotide sequence selected from the group consisting of SEQ ID NO:59-116, ii) a polynucleotide comprising a naturally occurring polynucleotide sequence at least 90% identical or at least about 90% identical to a polynucleotide sequence selected from the group consisting of SEQ ID NO:59-116, iii) a polynucleotide complementary to the polynucleotide of i), iv) a polynucleotide complementary to the polynucleotide of ii), and v) an RNA equivalent of i)-iv). Alternatively, the target polynucleotide can comprise a fragment of a polynucleotide selected from the group consisting of i)-v) above; c) quantifying the amount of hybridization complex; and d) comparing the amount of hybridization complex in the treated biological sample with the amount of hybridization complex in an untreated biological sample, wherein a difference in the amount of hybridization complex in the treated biological sample is indicative of toxicity of the test compound.

### BRIEF DESCRIPTION OF THE TABLES

Table 1 summarizes the nomenclature for full length polynucleotide and polypeptide embodiments of the invention.

Table 2 shows the GenBank identification number and annotation of the nearest GenBank homolog, and the PROTEOME database identification numbers and annotations of PROTEOME database homologs, for polypeptide embodiments of the invention. The probability scores for the matches between each polypeptide and its homolog(s) are also shown.

Table 3 shows structural features of polypeptide embodiments, including predicted motifs and domains, along with the methods, algorithms, and searchable databases used for analysis of the polypeptides.

Table 4 lists the cDNA and/or genomic DNA fragments which were used to assemble polynucleotide embodiments, along with selected fragments of the polynucleotides.

Table 5 shows representative cDNA libraries for polynucleotide embodiments.

Table 6 provides an appendix which describes the tissues and vectors used for construction of the cDNA libraries shown in Table 5.

Table 7 shows the tools, programs, and algorithms used to analyze polynucleotides and polypeptides, along with applicable descriptions, references, and threshold parameters.

### DESCRIPTION OF THE INVENTION

Before the present proteins, nucleic acids, and methods are described, it is understood that embodiments of the invention are not limited to the particular machines, instruments, materials, and methods described, as these may vary. It is also to be understood that the terminology used herein is for the purpose of describing particular embodiments only, and is not intended to limit the scope of the invention.

As used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with various embodiments of the invention. Nothing herein is to be construed as an admission that the invention is not entitled to antedate such disclosure by virtue of prior invention.

Definitions

“NAAP” refers to the amino acid sequences of substantially purified NAAP obtained from any species, particularly a mammalian species, including bovine, ovine, porcine, murine, equine,, and human, and from any source, whether natural, synthetic, semi-synthetic, or recombinant.

The term “agonist” refers to a molecule which intensifies or mimics the biological activity of NAAP. Agonists may include proteins, nucleic acids, carbohydrates, small molecules, or any other compound or composition which modulates the activity of NAAP either by directly interacting with NAAP or by acting on components of the biological pathway in which NAAP participates.

An “allelic variant” is an alternative form of the, gene encoding NAAP. Allelic variants may result from at least one mutation in the nucleic acid sequence and may result in altered mRNAs or in polypeptides whose structure or function may or may not be altered. A gene may have none, one, or many allelic variants of its naturally occurring form. Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding NAAP include those sequences with deletions, insertions, or substitutions of different nucleotides, resulting in a polypeptide the same as NAAP or a polypeptide with at least one functional characteristic of NAAP. Included within this definition are polymnorphisms which may or may not be readily detectable using a particular oligonucleotide probe of the polynucleotide encoding NAAP, and improper or unexpected hybridization to allelic variants, with a locus other than the normal chromosomal locus for the polynucleotide encoding NAAP. The encoded protein may also be “altered,” and may contain deletions, insertions, or substitutions of amino acid residues which produce a silent change and result in a functionally equivalent NAAP. Deliberate amino acid substitutions may be made on the basis of one or more similarities in polarity, charge, solubility, hydrophobicity, hydrophilicity, and/or the amphipathic nature of the residues, as long as the biological or immunological activity of NAAP is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valiue; glycine and alanine; and phenylalanine and tyrosine.

The terms “amino acid” and “amino acid sequence” can refer to an oligopeptide, a peptide, a polypeptide, or a protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete-native amino acid sequence associated with the recited protein molecule.

“Amplification” relates to the production of additional copies of a nucleic acid. Amplification may be carried out using polymerase chain reaction (PCR) technologies or other nucleic acid amplification technologies well known in the art.

The term “antagonist” refers to a molecule which inhibits or attenuates the biological activity of NAAP. Antagonists may include proteins such as antibodies, anticalins, nucleic acids, carbohydrates, small molecules, or any other compound or composition which modulates the activity of NAAP either by directly interacting with NAAP or by acting on components of the biological pathway in which NAAP participates.

The term “antibody” refers to intact immunoglobulin molecules as well as to fragments thereof, such as Fab, F(ab′)₂, and Fv fragments, which are capable of binding an epitopic determinant. Antibodies that bind NAAP polypeptides can be prepared using intact polypeptides or using fragments containing small peptides of interest as the immunizing antigen. The polypeptide or oligopeptide used to inmmunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antigenic determinant” refers to that region of a molecule (i.e., an epitope) that makes contact with a particular antibody. When a protein or a fragment of a protein is used to immunize a host animal, numerous regions of the protein may induce the production of antibodies which bind specifically to antigenic determinants (particular regions or three-dimensional structures on the protein). An antigenic determinant may compete with the intact antigen (i.e., the immunogen used to elicit the immune response) for binding to an antibody.

The term “aptamer” refers to a nucleic acid or oligonucleotide molecule that binds to a specific molecular target. Aptamers are derived from an in vitro evolutionary process (e.g., SELEX (Systematic Evolution of Ligands by Exponential Enrichment), described in U.S. Pat. No. 5,270,163), which selects for target-specific aptamer sequences from large combinatorial libraries. Aptamer compositions may be double-stranded or single-stranded, and may include deoxyribonucleotides, ribonucleotides, nucleotide derivatives, or other nucleotide-like molecules. The nucleotide components of an aptamer may have modified sugar groups (e.g., the 2′-OH group of a ribonucleotide may be replaced by 2′-F or 2′-NH₂), which may improve a desired property, e.g., resistance to nucleases or longer lifetime in blood. Aptamers may be conjugated to other molecules, e.g., a high molecular weight carrier to slow clearance of the aptamer from the circulatory system. Aptamers may be specifically cross-linked to their cognate ligands, e.g., by photo-activation of a cross-linker (Brody, E. N. and L. Gold (2000) J. Biotechnol. 74:5-13).

The term “intramer” refers to an aptamer which is expressed in vivo. For example, a vaccinia virus-based RNA expression system has been used to express specific RNA aptamers at high levels in the cytoplasm of leukocytes (Blind, M. et al. (1999) Proc. Natl. Acad. Sci. USA 96:3606-3610).

The term “spiegelmer” refers to an aptamer which includes L-DNA, L-RNA, or other left-handed nucleotide derivatives or nucleotide-like molecules. Aptamers containig left-handed nucleotides are resistant to degradation by naturally occurring enzymes, which normally act on substrates containing right-handed nucleotides.

The term “antisense” refers to any composition capable of base-pairing with the “sense” (coding) strand of a polynucleotide having a specific nucleic acid sequence. Antisense compositions may include DNA; RNA; peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may be produced by any method including chemical synthesis or transcription. Once introduced into a cell, the complementary antisense molecule base-pairs with a naturally occurring nucleic acid sequence produced by the cell to form duplexes which block either transcription or translation. The designation “negative” or “minus” can refer to the antisense strand, and the designation “positive” or “plus” can refer to the sense strand of a reference DNA molecule.

The term “biologically active” refers to a protein having structural, regulatory, or biochemical functions of a naturally occurring molecule. Likewise, “immunologically active” or “immunogenic” refers to the capability of the natural, recombinant, or synthetic NAAP, or of any oligopeptide thereof, to induce a specific immune response in appropriate animals or cells and to bind with specific antibodies.

“Complementary” describes the relationship between two single-stranded nucleic acid sequences that anneal by base-pairing. For example, 5′-AGT-3′ pairs with its complement, 3′-TCA-5′.

A “composition comprising a given polynucleotide” and a “composition comprising a given polypeptide” can refer to any composition containing the given polynucleotide or polypeptide. The composition may comprise a dry formulation or an aqueous solution. Compositions comprising polynucleotides encoding NAAP or fragments of NAAP may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g., sodium dodecyl sulfate; SDS), and other components (e.g., Denhardt's solution, dry milk, salmon sperm DNA, etc.).

“Consensus sequence” refers to a nucleic acid sequence which has been subjected to repeated DNA sequence analysis to resolve uncalled bases, extended using the XL-PCR kit (Applied Biosystems, Foster City Calif.) in the 5′ and/or the 3′ direction, and resequenced, or which has been assembled from one or more overlapping cDNA, EST, or genormic DNA fragments using a computer program for fragment assembly, such as the GELVIEW fragment assembly system (Accelrys, Burlington Mass.) or Phrap (University of Washington, Seattle Wash.). Some sequences have been both extended and assembled to produce the consensus sequence.

“Conservative amino acid substitutions” are those substitutions that are predicted to least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides.

The term “derivative” refers to a chemically modified polynucleotide or polypeptide. Chemical modifications of a polynucleotide can include, for example, replacement of hydrogen by an alkyl, acyl, hydroxyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “detectable label” refers to a reporter molecule or enzyme that is capable of generating a measurable signal and is covalently or noncovalently joined to a polynucleotide or polypeptide.

“Differential expression” refers to increased or upregulated; or decreased, downregulated, or absent gene or protein expression, determined by comparing at least two different samples. Such comparisons may be carried out between, for example, a treated and an untreated sample, or a diseased and a normal sample.

“Exon shuffling” refers to the recombination of different coding regions (exons). Since an exon may represent a structural or functional domain of the encoded protein, new proteins may be assembled through the novel reassortment of stable substructures, thus allowing acceleration of the evolution of new protein functions.

A “fragment” is a unique portion of NAAP or a polynucleotide encoding NAAP which can be identical in sequence to, but shorter in length than, the parent sequence. A fragment may comprise up to the entire length of the defined sequence, minus one nucleotide/amino acid residue. For example, a fragment may comprise from about 5 to about 1000 contiguous nucleotides or amino acid residues. A fragment used as a probe, primer, antigen, therapeutic molecule, or for other purposes, may be at least 5, 10, 15, 16, 20, 25, 30, 40, 50, 60, 75, 100, 150, 250 or at least 500 contiguous nucleotides or amino acid residues in length. Fragments may be preferentially selected from certain regions of a molecule. For example, a polypeptide fragment may comprise a certain length of contiguous amino acids selected from the first 250 or 500 amino acids (or first 25% or 50%) of a polypeptide as shown in a certain defined sequence. Clearly these lengths are exemplary, and any length that is supported by the specification, including the Sequence Listing, tables, and figures, may be encompassed by the present embodiments.

A fragment of SEQ ID NO:59-116 can comprise a region of unique polynucleotide sequence that specifically identifies SEQ ID NO:59-116, for example, as distinct from any other sequence in the genome from which the fragment was obtained. A fragment of SEQ ID NO:59-116 can be employed in one or more embodiments of methods of the invention, for example, in hybridization and amplification technologies and in analogous methods that distinguish SEQ ID NO:59-116 from related polynucleotides. The precise length of a fragment of SEQ ID NO:59-116 and the region of SEQ ID NO:59-116 to which the fragment corresponds are routinely determinable by one of ordinary skill in the art based on the intended purpose for the fragment.

A fragment of SEQ ID NO:1-58 is encoded by a fragment of SEQ ID NO:59-116. A fragment of SEQ ID NO:1-58 can comprise a region of unique amino acid sequence that specifically identifies SEQ ID NO:1-58. For example, a fragment of SEQ ID NO:1-58 can be used as an immunogenic peptide for the development of antibodies that specifically recognize SEQ ID NO:1-58. The precise length of a fragment of SEQ ID NO:1-58 and the region of SEQ ID NO:1-58 to which the fragment corresponds can be determined based on the intended purpose for the fragment using one or more analytical methods described herein or otherwise known in the art.

A “full length” polynucleotide is one containing at least a translation initiation codon (e.g., methionine) followed by an open reading frame and a translation termination codon. A “full length” polynucleotide sequence encodes a “full length” polypeptide sequence.

“Homology” refers to sequence similarity or, alternatively, sequence identity, between two or more polynucleotide sequences or two or more polypeptide sequences.

The terms “percent identity” and “% identity,” as applied to polynucleotide sequences, refer to the percentage of identical residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using one or more computer algorithms or programs known in the art or described herein. For example, percent identity can be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and P. M. Sharp (1989; CABIOS 5:151-153) and in Higgins, D. G. et al. (1992; CABIOS 8:189-191). For pairwise alignments of polynucleotide sequences, the default parameters are set as follows: Ktuple=2, gap penalty=5, window=4, and “diagonals saved”=4. The “weighted” residue weight table is selected as the default.

Alternatively, a suite of commonly used and freely available sequence comparison algorithms which can be used is provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at http://www.ncbi.nlm.nih.gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at http://www.ncbi.nlm.nih.gov/gorf/b12.html. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.12 (Apr. 21, 2000) set at default parameters. Such default parameters maybe, for example:
Matrix: BLOSUM62
Reward for match: 1
Penalty for mismatch: −2
Open Gap: 5 and Extension Gap: 2 penalties
Gap×drop-off: 50
Expect: 10
Word Size: 11
Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of identical residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. The phrases “percent similarity” and “% similarity,” as applied to polypeptide sequences, refer to the percentage of residue matches, including identical residue matches and conservative substitutions, between at least two polypeptide sequences aligned using a standardized algorithm. In contrast, conservative substitutions are not included in the calculation of percent identity between polypeptide sequences.

Percent identity between polypeptide sequences may be determined using the default parameters of the CLUSTAL V algoritun as incorporated into the MEGALIGN version 3.12e sequence alignment program (described and referenced above). For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table.

Alternatively the NCBI BLAST software suite may be used. For example, for a pairwise comparison of two polypeptide sequences, one may use the “BLAST 2 Sequences” tool Version 2.0.12 (Apr. 21, 2000) with blastp set at default parameters. Such default parameters may be, for example:
Matrix: BLOSUM62
Open Gap: 11 and Extension Gap: 1 penalties
Gap×drop-off: 50
Expect: 10
Word Size: 3
Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

“Human artificial chromosomes” (HACs) are linear microchromosomes which may contain DNA sequences of about 6 kb to 10 Mb in size and which contain all of the elements required for chromosome replication, segregation and maintenance.

The term “humanized antibody” refers to an antibody molecule in which the amino acid sequence in the non-antigen binding regions has been altered so that the antibody more closely resembles a human antibody, and still retains its original binding ability.

“Hybridization” refers to the process by which a polynucleotide strand anneals with a complementary strand through base pairing under defined hybridization conditions. Specific hybridization is an indication that two nucleic acid sequences share a high degree of complementarity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C. in the presence of about 6×SSC, about 1% (w/v) SDS, and about 100 μg/ml sheared, denatured salmon sperm DNA.

Generally, stringency of hybridization is expressed, in part, with reference to the temperature under which the wash step is carried out. Such wash temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook, J. and D. W. Russell (2001; Molecular Cloning: A Laboratory Manual, 3rd ed., vol. 1-3, Cold Spring Harbor Press, Cold Spring Harbor N.Y., ch. 9).

High stringency conditions for hybridization between polynucleotides of the present invention include wash conditions of 68° C. in the presence of about 0.2×SSC and about 0.1% SDS, for 1 hour. Alternatively, temperatures of about 65° C., 60° C., 55° C., or 42° C. may be used. SSC concentration may be varied from about 0.1 to 2×SSC, with SDS being present at about 0.1%. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

The term “hybridization complex” refers to a complex formed between two nucleic acids by virtue of the formation of hydrogen bonds between complementary bases. A hybridization complex may be formed in solution (e.g., Cot or Rot analysis) or formed between one nucleic acid present in solution and another nucleic acid immobilized on a solid support (e.g., paper, membranes, filters, chips, pins or glass slides, or any other appropriate substrate to which cells or their nucleic acids have been fixed).

The words “insertion” and “addition” refer to changes in an amino acid or polynucleotide sequence resulting in the addition of one or more amino acid residues or nucleotides, respectively.

“Immune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

An “immunogenic fragment” is a polypeptide or oligopeptide fragment of NAAP which is capable of eliciting an immune response when introduced into a living organism, for example, a mammal. The term “immunogenic fragment” also includes any polypeptide or oligopeptide fragment of NAAP which is useful in any of the antibody production methods disclosed herein or known in the art.

The term “microarray” refers to an arrangement of a plurality of polynucleotides, polypeptides, antibodies, or other chemical compounds on a substrate.

The terms “element” and “array element” refer to a polynucleotide, polypeptide, antibody, or other chemical compound having a unique and defined position on a microarray.

The term “modulate” refers to a change in the activity of NAAP. For example, modulation may cause an increase or a decrease in protein activity, binding characteristics, or any other biological, functional, or inmmunological properties of NAAP.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

“Peptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

“Post-translational modification” of an NAAP may involve lipidation, glycosylation, phosphorylation, acetylation, racemization, proteolytic cleavage, and other modifications known in the art. These processes may occur synthetically or biochemically. Biochemical modifications will vary by cell type depending on the enzymatic milieu of NAAP.

“Probe” refers to nucleic acids encoding NAAP, their complements, or fragments thereof, which are used to detect identical, allelic or related nucleic acids. Probes are isolated oligonucleotides or polynucleotides attached to a detectable label or reporter molecule. Typical labels include radioactive isotopes, ligands, chemiluminescent agents, and enzymes. “Primers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 25, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the tables, figures, and Sequence Listing, may be used.

Methods for preparing and using probes and primers are described in, for example, Sambrook, J. and D. W. Russell (2001; Molecular Cloning: A Laboratory Manual, 3rd ed., vol. 1-3, Cold Spring Harbor Press, Cold Spring Harbor N.Y.), Ausubel, F. M. et al. (1999; Short Protocols in Molecular Biology, 4th ed., John Wiley & Sons, New York N.Y.), and Innis, M. et al. (1990; PCR Protocols, A Guide to Methods and Applications, Academic Press, San Diego Calif.). PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/MIT Center for Genome Research, Cambridge Mass.) allows the user to input a “mispriming library,” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

A “recombinant nucleic acid” is a nucleic acid that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook and Russell (supra). The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the mammal.

A “regulatory element” refers to a nucleic acid sequence usually derived from untranslated regions of a gene and includes enhancers, promoters, introns, and 5′ and 3′ untranslated regions (UTRs). Regulatory elements interact with host or viral proteins which control transcription, translation, or RNA stability.

“Reporter molecules” are chemical or biochemical moieties used for labeling a nucleic acid, amino acid, or antibody. Reporter molecules include radionuclides; enzymes; fluorescent, chemiluminescent, or chromogenic agents; substrates; cofactors; inhibitors; magnetic particles; and other moieties known in the art.

An “RNA equivalent,” in reference to a DNA molecule, is composed of the same linear sequence of nucleotides as the reference DNA molecule with the exception that all occurrences of the nitrogenous base thymine are replaced with uracil, and the sugar backbone is composed of ribose instead of deoxyribose.

The term “sample” is used in its broadest sense. A sample suspected of containing NAAP, nucleic acids encoding NAAP, or fragments thereof may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide comprising the epitope A, or the presence of free unlabeled A, in a reaction containing free labeled A and the antibody will reduce the amount of labeled A that binds to the antibody.

The term “substantially purified” refers to nucleic acid or amino acid sequences that are removed from their natural environment and are isolated or separated, and are at least about 60% free, preferably at least about 75% free, and most preferably at least about 90% free from other components with which they are naturally associated.

A “substitution” refers to the replacement of one or more amino acid residues or nucleotides by different amino acid residues or nucleotides, respectively.

“Substrate” refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

A “transcript image” or “expression profile” refers to the collective pattern of gene expression by a particular cell type or tissue under given conditions at a given time.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

A “transgenic organism,” as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. In another embodiment, the nucleic acid can be introduced by infection with a recombinant viral vector, such as a lentiviral vector (Lois, C. et al. (2002) Science 295:868-872). The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, plants and animals. The isolated DNA of the present invention can be introduced into the host by methods known in the art, for example infection, transfection, transformation or transconjugation. Techniques for transferring the DNA of the present invention into such organisms are widely known and provided in references such as Sambrook and Russell (supra).

A “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0.9 (May 7, 1999) set at default parameters. Such a pair of nucleic acids may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length. A variant may be described as, for example, an “allelic” (as defined above), “splice,” “species,” or “polymorphic” variant. A splice variant may have significant identity to a reference molecule, but will generally have a greater or lesser number of polynucleotides due to alternate splicing of exons during mRNA processing. The corresponding polypeptide may possess additional functional domains or lack domains that are present in the reference molecule. Species variants are polynucleotides that vary from one species to another. The resulting polypeptides will generally have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity or sequence sirnilarity to the particular polypeptide sequence over a certain length of one of the polypeptide sequences using blastp with the “BLAST 2 Sequences” tool Version 2.0.9 (May 7, 1999) set at default parameters. Such a pair of polypeptides may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity or sequence similarity over a certain defined length of one of the polypeptides.

The Invention

Various embodiments of the invention include new human nucleic acid-associated proteins (NAAP), the polynucleotides encoding NAAP, and the use of these compositions for the diagnosis, treatment, or prevention of cell proliferative, neurological, developmental, and autoimmune/inflammatory disorders, and infections.

Table 1 summarizes the nomenclature. for the full length polynucleotide and polypeptide embodiments of the invention. Each polynucleotide and its corresponding polypeptide are correlated to a single Incyte project identification number (Incyte Project ID). Each polypeptide sequence is denoted by both a polypeptide sequence identification number (Polypeptide SEQ ID NO:) and an Incyte polypeptide sequence number (Incyte Polypeptide ID) as shown. Each polynucleotide sequence is denoted by both a polynucleotide sequence identification number (Polynucleotide SEQ ID NO:) and an Incyte polynucleotide consensus sequence number (Incyte Polynucleotide ID) as shown. Column 6 shows the Incyte ID numbers of physical, full length clones corresponding to the polypeptide and polynucleotide sequences of the invention. The full length clones encode polypeptides which have at least 95% sequence identity to the polypeptide sequences shown in column 3.

Table 2 shows sequences with homology to polypeptide embodiments of the invention as identified by BLAST analysis against the GenBank protein (genpept) database and the PROTEOME database. Columns 1 and 2 show the polypeptide sequence identification number (Polypeptide SEQ ID NO:) and the corresponding Incyte polypeptide sequence number (Incyte Polypeptide ID) for polypeptides of the invention. Column 3 shows the GenBank identification number (GenBank ID NO:) of the nearest GenBank homolog and the PROTEOME database identification numbers (PROTEOME ID NO:) of the nearest PROTEOME database homologs. Column 4 shows the probability scores for the matches between each polypeptide and its homolog(s). Column 5 shows the annotation of the GenBank and PROTEOME database homolog(s) along with relevant citations where applicable, all of which are expressly incorporated by reference herein.

Table 3 shows various structural features of the polypeptides of the invention. Columns 1 and 2 show the polypeptide sequence identification number (SEQ ID NO:) and the corresponding Incyte polypeptide sequence number (Incyte Polypeptide ID) for each polypeptide of the invention. Column 3 shows the number of amino acid residues in each polypeptide. Column 4 shows potential phosphorylation sites, and column 5 shows potential glycosylation sites, as determined by the MOTIFS program of the GCG sequence analysis software package (Accelrys, Burlington Mass.). Column 6 shows amino acid residues comprising signature sequences, domains, and motifs. Column 7 shows analytical methods for protein structure/function analysis and in some cases, searchable databases to which the analytical methods were applied.

Together, Tables 2 and 3 summarize the properties of polypeptides of the invention, and these properties establish that the claimed polypeptides are nucleic acid-associated proteins. For example, SEQ ID NO:2 is 57% identical, from residue T192 to residue T586, to human DNA binding protein (GenBank ID g1020145) as determined by the Basic Local Alignment Search Tool (BLAST). (See Table 2.) The BLAST probability score is 1.1e-149, which indicates the probability of obtaining the observed polypeptide sequence alignment by chance. SEQ ID NO:2 is localized to the nucleus, binds DNA, and is a zinc finger protein containing a KRAB domain, as determined by BLAST analysis using the PROTEOME database. SEQ ID NO:2 also contains a KRAB box domain and 14 zinc finger, C2H2 type, domains as determined by searching for statistically significant matches in the hidden Markov model (HMM)-based PFAM database of conserved protein family domains. (See Table 3.) Data from BLIMPS, MOTIFS, and other BLAST analyses provide further corroborative evidence that SEQ ID NO:2 is a KRAB family zinc finger protein.

In an alternative example, SEQ ID NO:16 is 93% identical, from residue Ml to residue R364, to chicken transcription factor, LEF-l (GenBank ID g3258665) as determined by the Basic Local Alignment Search Tool (BLAST). (See Table 2.) The BLAST probability score is 9.8e-191, which indicates the probability of obtaining the observed polypeptide sequence alignment by chance. SEQ ID NO:16 is localized to the nucleus, functions as a DNA-binding protein, and is a transcriptional activator, as determined by BLAST analysis using the PROTEOME database. SEQ ID NO:16 also contains a HMG (high mobility group) box domain as determined by searching for statistically significant matches in the hidden Markov model (HMM)-based PFAM database of conserved protein family domains. (See Table 3.) Data from BLIMPS, and other BLAST analyses provide further corroborative evidence that SEQ ID NO:16 is a LEF-1 transcription factor.

In an alternative example, SEQ ID NO:19 is 71% identical from residue H19 to residue A1 13, and 100% identical from residue Ml to residue Y48, to ribosomal protein L27a (GenBank ID g550⁰¹7) as determined by the Basic Local Alignment Search Tool (BLAST). (See Table 2.) The BLAST probability score is 1.4e-3 1, which indicates the probability of obtaining the observed polypeptide sequence alignment by chance. SEQ ID NO:19 is a component of the large 60S ribosomal subunit, and is abnormally expressed in colorectal carcinomas, as determined by BLAST analysis using the PROTEOME database. SEQ ID NO:19 also contains a ribosomal protein L15 domain as determined by searching for statistically significant matches in the hidden Markov model (HMM)-based PFAM database of conserved protein family domains. (See Table 3.) Data from BLIMPS, MOTIFS, and PROFILESCAN analyses provide further corroborative evidence that SEQ ID NO:19 is a ribosomal protein.

In an alternative example, SEQ ID NO:51 is 98% identical, from residue MI to residue H477, to a human transcription factor (GenBank ID g516381) as determined by the Basic Local Alignment Search Tool (BLAST). (See Table 2.) The BLAST probability score is 3.5e-266, which indicates the probability of obtaining the observed polypeptide sequence alignment by chance. SEQ ID NO:5 1 also has homology to proteins that are localized to the neuronal cells, have DNA-binding and transcriptional regulation function, and are fork head proteins, as determined by BLAST analysis using the PROTEOME database. SEQ ID NO:51 also contains a fork head domain as determined by searching for statistically significant matches in the hidden Markov model (HMM)-based PFAM database of conserved protein family domains. (See Table 3.) Data from BLIMPS, MOTIFS, and PROFHLESCAN analyses provide further corroborative evidence that SEQ ID NO:51 is a fork head DNA-binding protein.

SEQ ID NO:1, SEQ ID NO:3-15, SEQ ID NO:17-18, SEQ ID NO:20-50, and SEQ ID NO:52-58 were analyzed and annotated in a similar manner. The algorithms and parameters for the analysis of SEQ ID NO:1-58 are described in Table 7.

As shown in Table 4, the full length polynucleotide embodiments were assembled using cDNA sequences or coding (exon) sequences derived from genomic DNA, or any combination of these two types of sequences. Column 1 lists the polynucleotide sequence identification number (Polynucleotide SEQ ID NO:), the corresponding Incyte polynucleotide consensus sequence number (Incyte ID) for each polynucleotide of the invention, and the length of each polynucleotide sequence in basepairs. Column 2 shows the nucleotide start (5′) and stop (3′) positions of the cDNA and/or genomic sequences used to assemble the full length polynucleotide embodiments, and of fragments of the polynucleotides which are useful, for example, in hybridization or amplification technologies that identify SEQ ID NO:59-116 or that distinguish between SEQ ID NO:59-116 and related polynucleotides.

The polynucleotide fragments described in Column 2 of Table 4 may refer specifically, for example, to Incyte cDNAs derived from tissue-specific cDNA libraries or from pooled cDNA libraries. Alternatively, the polynucleotide fragments described in column 2 may refer to GenBank cDNAs or ESTs which contributed to the assembly of the full length polynucleotides. In addition, the polynucleotide fragments described in column 2 may identify sequences derived from the ENSEMBL (The Sanger Centre, Cambridge, UK) database (i.e., those sequences including the designation “ENST”). Alternatively, the polynucleotide fragments described in column 2 may be derived from the NCBI RefSeq Nucleotide Sequence Records Database (i.e., those sequences including the designation “NM” or “NT”) or the NCBI RefSeq Protein Sequence Records (i. e., those sequences including the designation “NP”). Alternatively, the polynucleotide fragments described in column 2 may refer to assemblages of both cDNA and Genscan-predicted exons brought together by an “exon stitching” algorithm. For example, a polynucleotide sequence identified as FL_XXXKKK_N₁_{_{—}}N₂_{_{—}}YYYYY_N₃_{_{—}}N₄ represents a “stitched” sequence in which XXXXXX is the identification number of the cluster of sequences to which the algorithm was applied, and YYYYY is the number of the prediction generated by the algorithm, and N_{1,2,3 . . .} , if present, represent specific exons that may have been manually edited during analysis (See Example V). Alternatively, the polynucleotide fragments in column 2 may refer to assemblages of exons brought together by an “exon-stretching” algorithm. For example, a polynucleotide sequence identified as FLXXXXXX_gAAAAA_gBBBBB_{—}1_N is a “stretched” sequence, with XXXXXX being the Incyte project identification number, gAAAAA being the GenBank identification number of the human genomic sequence to which the “exon-stretching” algorithm was applied, gBBBBB being the GenBank identification number or NCBI RefSeq identification number of the nearest GenBank protein homolog, and N referring to specific exons (See Example V). In instances where a RefSeq sequence was used as a protein homolog for the “exon-stretching” algorithm, a RefSeq identifier (denoted by “NM,” “NP,” or “NT”) maybe used in place of the GenBank identifier (i.e., gBBBBB).

Alternatively, a prefix identifies component sequences that were hand-edited, predicted from genomic DNA sequences, or derived from a combination of sequence analysis methods. The following Table lists examples of component sequence prefixes and corresponding sequence analysis methods associated with the prefixes (see Example IV and Example V).

| Prefix | Type of analysis and/or examples of programs |
|---|---|
| GNN, GFG, | Exon prediction from genomic sequences using, for |
| ENST | example, GENSCAN (Stanford University, CA, USA) or |
| | FGENES (Computer Genomics Group, The Sanger Centre, |
| | Cambridge, UK). |
| GBI | Hand-edited analysis of genomic sequences. |
| FL | Stitched or stretched genomic sequences (see Example V). |
| INCY | Full length transcript and exon prediction from mapping of |
| | EST sequences to the genome. Genomic location and EST |
| | composition data are combined to predict the exons and |
| | resulting transcript. |

In some cases, Incyte cDNA coverage redundant with the sequence coverage shown in Table 4 was obtained to confirm the final consensus polynucleotide sequence, but the relevant Incyte cDNA identification numbers are not shown.

Table 5 shows the representative cDNA libraries for those full length polynucleotides which were assembled using Incyte cDNA sequences. The representative cDNA library is the Incyte cDNA library which is most frequently represented by the Incyte cDNA sequences which were used to assemble and confirm the above polynucleotides. The tissues and vectors which were used to construct the cDNA libraries shown in Table 5 are described in Table 6.

The invention also encompasses NAAP variants. Various embodiments of NAAP variants can have at least about 80%, at least about 90%, or at least about 95% amino acid sequence identity to the NAAP amino acid sequence, and can contain at least one functional or structural characteristic of NAAP.

Various embodiments also encompass polynucleotides which encode NAAP. In a particular embodiment, the invention encompasses a polynucleotide sequence comprising a sequence selected from the group consisting of SEQ ID NO:59-116, which encodes NAAP. The polynucleotide sequences of SEQ ID NO:59-116, as presented in the Sequence Listing, embrace the equivalent RNA sequences, wherein occurrences of the nitrogenous base thymine are replaced with uracil, and the sugar backbone is composed of ribose instead of deoxyribose.

The invention also encompasses variants of a polynucleotide encoding NAAP. In particular, such a variant polynucleotide will have at least about 70%, or alternatively at least about 85%, or even at least about 95% polynucleotide sequence identity to a polynucleotide encoding NAAP. A particular aspect of the invention encompasses a variant of a polynucleotide comprising a sequence selected from the group consisting of SEQ ID NO:59-116 which has at least about 70%, or alternatively at least about 85%, or even at least about 95% polynucleotide sequence identity to a nucleic acid sequence selected from the group consisting of SEQ ID NO:59-116. Any one of the polynucleotide variants described above can encode a polypeptide which contains at least one functional or structural characteristic of NAAP.

In addition, or in the alternative, a polynucleotide variant of the invention is a splice variant of a polynucleotide encoding NAAP. A splice variant may have portions which have significant sequence identity to a polynucleotide encoding NAAP, but will generally have a greater or lesser number of polynucleotides due to additions or deletions of blocks of sequence arising from alternate splicing of exons during mRNA processing. A splice variant may have less than about 70%, or alternatively less than about 60%, or alternatively less than about 50% polynucleotide sequence identity to a polynucleotide encoding NAAP over its entire length; however, portions of the splice variant will have at least about 70%, or alternatively at least about 85%, or alternatively at least about 95%, or alternatively 100% polynucleotide sequence identity to portions of the polynucleotide encoding NAAP. For example, a polynucleotide comprising a sequence of SEQ ID NO:105 and a polynucleotide comprising a sequence of SEQ ID NO:110 are splice variants of each other. Any one of the splice variants described above can encode a polypeptide which contains at least one functional or structural characteristic of NAAP.

It will be appreciated by those skilled in the art that as a result of the degeneracy of the genetic code, a multitude of polynucleotide sequences encoding NAAP, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring NAAP, and all such variations are to be considered as being specifically disclosed.

Although polynucleotides which encode NAAP and its variants are generally capable of hybridizing to polynucleotides encoding naturally occurring NAAP under appropriately selected conditions of stringency, it may be advantageous to produce polynucleotides encoding NAAP or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding NAAP and its derivatives without altering the encoded amino acid sequences include the production of RNA transcripts having more desirable properties, such as a greater half-life, than transcripts produced from the naturally occurring sequence.

The invention also encompasses production of polynucleotides which encode NAAP and NAAP derivatives, or fragments thereof, entirely by synthetic chemistry. After production, the synthetic polynucleotide may be inserted into any of the many available expression vectors and cell systems using reagents well known in the art. Moreover, synthetic chemistry may be used to introduce mutations into a polynucleotide encoding NAAP or any fragment thereof.

Embodiments of the invention can also include polynucleotides that are capable of hybridizing to the claimed polynucleotides, and, in particular, to those having the sequences shown in SEQ ID NO:59-116 and fragments thereof, under various conditions of stringency (Wahl, G. M. and S. L. Berger (1987) Methods Enzymol. 152:399407; Kimmel, A. R. (1987) Methods Enzymol. 152:507-511). Hybridization conditions, including annealing and wash conditions, are described in “Definitions.”

Methods for DNA sequencing are well known in the art and may be used to practice any of the embodiments of the invention. The methods may employ such enzymes as the Klenow fragment of DNA polymerase 1, SEQUENASE (US Biochemical, Cleveland Ohio), Taq polymerase (Applied Biosystems), thermostable T7 polymerase (Amersham Biosciences, Piscataway N.J.), or combinations of polymerases and proofreading exonucleases such as those found in the ELONGASE amplification system (Invitrogen, Carlsbad Calif.). Preferably, sequence preparation is automated with machines such as the MICROLAB 2200 liquid transfer system (Hamilton, Reno Nev.), PTC200 thermal cycler (MJ Research, Watertown Mass.) and ABI CATALYST 800 thermal cycler (Applied Biosystems). Sequencing is then carried out using either the ABI 373 or 377 DNA sequencing system (Applied Biosystems), the MEGABACE 1000 DNA sequencing system (Amersham Biosciences), or other systems known in the art. The resulting sequences are analyzed using a variety of algorithms which are well known in the art (Ausubel et al., supra, ch. 7; Meyers, R. A. (1995) Molecular Biology and Biotechnology, Wiley V C H, New York N.Y., pp. 856-853).

The nucleic acids encoding NAAP may be extended utilizing a partial nucleotide sequence and employing various PCR-based methods known in the art to detect upstream sequences, such as promoters and regulatory elements. For example, one method which may be employed, restriction-site PCR, uses universal and nested primers to amplify unknown sequence from genomic DNA within a cloning vector (Sarkar, G. (1993) PCR Methods Applic. 2:318-322). Another method, inverse PCR, uses primers that extend in divergent directions to amplify unknown sequence from a circularized template. The template is derived from restriction fragments comprising a known genomic locus and surrounding sequences (Triglia, T. et al. (1988) Nucleic Acids Res. 16:8186). A third method, capture PCR, involves PCR amplification of DNA fragments adjacent to known sequences in human and yeast artificial chromosome DNA (Lagerstrom, M. et al. (1991) PCR Methods Applic. 1:111-119). In this method, multiple restriction enzyme digestions and ligations may be used to insert an engineered double-stranded sequence into a region of unknown sequence before performing PCR. Other methods which may be used to retrieve unknown sequences are known in the art (Parker, J. D. et al. (1991) Nucleic Acids Res. 19:3055-3060). Additionally, one may use PCR, nested primers, and PROMOTERFINDER libraries (Clontech, Palo Alto Calif.) to walk genomic DNA. This procedure avoids the need to screen libraries and is useful in finding intron/exon junctions. For all PCR-based methods, primers maybe designed using commercially available software, such as OLIGO 4.06 primer analysis software (National Biosciences, Plymouth Minn.) or another appropriate program, to be about 22 to 30 nucleotides in length, to have a GC content of about 50% or more, and to anneal to the template at temperatures of about 68° C. to 72° C.

When screening for full length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., GENOTYPER and SEQUENCE NAVIGATOR, Applied Biosystems), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotides or fragments thereof which encode NAAP may be cloned in recombinant DNA molecules that direct expression of NAAP, or fragments or functional equivalents thereof, in appropriate host cells. Due to the inherent degeneracy of the genetic code, other polynucleotides which encode substantially the same or a functionally equivalent polypeptides may be produced and used to express NAAP.

The polynucleotides of the invention can be engineered using methods generally known in the art in order to alter NAAP-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

The nucleotides of the present invention may be subjected to DNA shuffling techniques such as MOLECULARBREEDING (Maxygen Inc., Santa Clara Calif.; described in U.S. Pat. No. 5,837,458; Chang, C.-C. et al. (1999) Nat. Biotechnol. 17:793-797; Christians, F. C. et al. (1999) Nat. Biotechnol. 17:259-264; and Crameri, A. et al. (1996) Nat. Biotechnol. 14:315-319) to alter or improve the biological properties of NAAP, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

In another embodiment, polynucleotides encoding NAAP may be synthesized, in whole or in part, using one or more chemical methods well known in the art (Caruthers, M. H. et al. (1980) Nucleic Acids Symp. Ser. 7:215-223; Horn, T. et al. (1980) Nucleic Acids Symp. Ser. 7:225-232). Alternatively, NAAP itself or a fragment thereof may be synthesized using chemical methods known in the art. For example, peptide synthesis can be performed using various solution-phase or solid-phase techniques (Creighton, T. (1984) Proteins, Structures and Molecular Properties, W H Freeman, New York N.Y., pp. 55-60; Roberge, J. Y. et al. (1995) Science 269:202-204). Automated synthesis may be achieved using the ABI 43 1A peptide synthesizer (Applied Biosystems). Additionally, the amino acid sequence of NAAP, or any part thereof, may be altered during direct synthesis and/or combined with sequences from other proteins, or any part thereof, to produce a variant polypeptide or a polypeptide having a sequence of a naturally occurring polypeptide.

The peptide may be substantially purified by preparative high performance liquid chromatography (Chiez, R. M. and F. Z. Regnier (1990) Methods Enzymol. 182:392-421). The composition of the synthetic peptides may be confirmed by amino acid analysis or by sequencing (Creighton, supra, pp. 28-53).

In order to express a biologically active NAAP, the polynucleotides encoding NAAP or derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotides encoding NAAP. Such elements may vary in their strength and specificity. Specific initiation signals may also be used to achieve more efficient translation of polynucleotides encoding NAAP. Such signals include the ATG initiation codon and adjacent sequences, e.g. the Kozak sequence. In cases where a polynucleotide sequence encoding NAAP and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used (Scharf, D. et al. (1994) Results Probl. Cell Differ. 20:125-162).

Methods which are well known to those skilled in the art may be used to construct expression vectors containing polynucleotides encoding NAAP and appropriate transcriptional and translational control elements. These methods include in vitro recombinant DNA techniques, synthetic techniques, and in vivo genetic recombination (Sambrook and Russell, supra, ch. 1-4, and 8; Ausubel et al., supra, ch. 1, 3, and 15).

A variety of expression vector/host systems may be utilized to contain and express polynucleotides encoding NAAP. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems (Sambrook and Russell, supra; Ausubel et al., supra; Van Heeke, G. and S. M. Schuster (1989) J. Biol. Chem. 264:5503-5509; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; Harrington, J. J. et al. (1997) Nat. Genet. 15:345-355). Expression vectors derived from retroviruses, adenoviruses, or herpes or vaccinia viruses, or from various bacterial plasmids, may be used for delivery of polynucleotides to the targeted organ, tissue, or cell population (Di Nicola, M. et al. (1998) Cancer Gen. Ther. 5:350-356; Yu, M. et al. (1993) Proc. Natl. Acad. Sci. USA 90:6340-6344; Buller, R. M. et al. (1985) Nature 317:813-815; McGregor, D. P. et al. (1994) Mol. Immunol. 31:219-226; Verma, I. M. and N. Somia (1997) Nature 389:239-242). The invention is not limited by the host cell employed.

In bacterial systems, a number of cloning and expression vectors may be selected depending upon the use intended for polynucleotides encoding NAAP. For example, routine cloning, subcloning, and propagation of polynucleotides encoding NAAP can be achieved using a multifunctional E. coli vector such as PBLUESCRRPT (Stratagene, La Jolla Calif.) or PSPORT1 plasmid (Invitrogen). Ligation of polynucleotides encoding NAAP into the vector's multiple cloning site disrupts the lacZ gene, allowing a calorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence (Van Heeke, G. and S. M. Schuster (1989) J. Biol. Chem. 264:5503-5509). When large quantities of NAAP are needed, e.g. for the production of antibodies, vectors which direct high level expression of NAAP may be used. For example, vectors containing the strong, inducible SP6 or T7 bacteriophage promoter may be used.

Yeast expression systems may be used for production of NAAP. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign polynucleotide sequences into the host genome for stable propagation (Ausubel et al., supra; Bitter, G. A. et al. (1987) Methods Enzymol. 153:516-544; Scorer, C. A. et al. (1994) Bio/Technology 12:181-184).

Plant systems may also be used for expression of NAAP. Transcription of polynucleotides encoding NAAP may be driven by viral promoters, e.g., the 35S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters may be used (Coruzzi, G. et al. (1984) EMBO J. 3:1671-1680; Broglie, R. et al. (1984) Science 224:838-843; Winter, J. et al. (1991) Results Probl. Cell Differ. 17:85-105). These constructs can be introduced into plant cells by direct DNA transformation or pathogen-mediated transfection (The McGraw Hill Yearbook of Science and Technology (1992) McGraw Hill, New York N.Y., pp. 191-196).

In mammalian cells, a number of viral-based expression systems may be utilized. In cases where an adenovirus is used as an expression vector, polynucleotides encoding NAAP may be ligated into an adenovirus transcription/translation complex consisting of the late promoter and tripartite leader sequence. Insertion in a non-essential E1 or E3 region of the viral genome may be used to obtain infective virus which expresses NAAP in host cells (Logan, J. and T. Shenk (1984) Proc. Natl. Acad. Sci. USA 81:3655-3659). In addition, transcription enhancers, such as the Rous sarcoma virus (RSV) enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes (Harrington, J. J. et al. (1997) Nat. Genet. 15:345-355).

For long term production of recombinant proteins in mammalian systems, stable expression of NAAP in cell lines is preferred. For example, polynucleotides encoding NAAP can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk⁻ and apr⁻ cells, respectively (Wigler, M. et al. (1977) Cell 11:223-232; Lowy, I. et al. (1980) Cell 22:817-823). Also, antimetabolite, antibiotic, or herbicide resistance can be used as the basis for selection. For example, dhfr confers resistance to methotrexate; neo confers resistance to the arninoglycosides neomycin and G-418; and als and pat confer resistance to chlorsulfuron and phosphinotricin acetyltransferase, respectively (Wigler, M. et al. (1980) Proc. Natl. Acad. Sci. USA 77:3567-3570; Colbere-Garapin, F. et al. (1981) J. Mol. Biol. 150:1-14). Additional selectable genes have been described, e.g., trpB and hisD, which alter cellular requirements for metabolites (Hartman, S. C. and R. C. Mulligan (1988) Proc. Natl. Acad. Sci. USA 85:8047-8051). Visible markers, e.g., anthocyanins, green fluorescent proteins (GFP; Clontech), β-glucuronidase and its substrate β-glucuronide, or luciferase and its substrate luciferin may be used. These markers can be used not only to identify transformants, but also to quantify the amount of transient or stable protein expression attributable to a specific vector system (Rhodes, C. A. (1995) Methods Mol. Biol. 55:121-131).

Although the presence/absence of marker gene expression suggests that the gene of interest is also present, the presence and expression of the gene may need to be confirmed. For example, if the sequence encoding NAAP is inserted within a marker gene sequence, transformed cells containing polynucleotides encoding NAAP can be identified by the absence of marker gene function. Alternatively, a marker gene can be placed in tandem with a sequence encoding NAAP under the control of a single promoter. Expression of the marker gene in response to induction or selection usually indicates expression of the tandem gene as well.

In general, host cells that contain the polynucleotide encoding NAAP and that express NAAP may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of NAAP using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on NAAP is preferred, but a competitive binding assay may be employed. These and other assays are well known in the art (Hampton, R. et al. (1990) Serological Methods, a Laboratory Manual, APS Press, St. Paul Minn., Sect. IV; Coligan, J. E. et al. (1997) Current Protocols in Immunology, Greene Pub. Associates and Wiley-Interscience, New York N.Y.; Pound, J. D. (1998) Immunochemical Protocols, Humana Press, Totowa N.J.).

A wide variety of labels and conjugation techniques are known by those skilled in the art and may be used in various nucleic acid and amino acid assays. Means for producing labeled hybridization or PCR probes for detecting sequences related to polynucleotides encoding NAAP include oligolabeling, nick translation, end-labeling, or PCR amplification using a labeled nucleotide. Alternatively, polynucleotides encoding NAAP, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Amersham Biosciences, Promega (Madison Wis.), and US Biochemical. Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with polynucleotides encoding NAAP may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode NAAP may be designed to contain signal sequences which direct secretion of NAAP through a prokaryotic or eukaryotic cell membrane.

In addition, a host cell strain may be chosen for its ability to modulate expression of the inserted polynucleotides or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used to specify protein targeting, folding, and/or activity. Different host cells which have specific cellular machinery and characteristic mechanisms for post-translational activities (e.g., CHO, HeLa, MDCK, HEK293, and WI38) are available from the American Type Culture Collection (ATCC, Manassas Va.) and may be chosen to ensure the correct modification and processing of the foreign protein.

In another embodiment of the invention, natural, modified, or recombinant polynucleotides encoding NAAP may be ligated to a heterologous sequence resulting in translation of a fusion protein in any of the aforementioned host systems. For example, a chimeric NAAP protein containing a heterologous moiety that can be recognized by a commercially available antibody may facilitate the screening of peptide libraries for inhibitors of NAAP activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the NAAP encoding sequence and the heterologous protein sequence, so that NAAP may be cleaved away from the heterologous moiety following purification. Methods for fusion protein expression and purification are discussed in Ausubel et al. (supra, ch. 10 and 16). A variety of commercially available kits may also be used to facilitate expression and purification of fusion proteins.

In another embodiment, synthesis of radiolabeled NAAP may be achieved in vitro using the TNT rabbit reticulocyte lysate or wheat germ extract system (Promega). These systems couple transcription and translation of protein-coding sequences operably associated with the T7, T3, or SP6 promoters. Translation takes place in the presence of a radiolabeled amino acid precursor, for example, ³⁵S-methionine.

NAAP, fragments of NAAP, or variants of NAAP may be used to screen for compounds that specifically bind to NAAP. One or more test compounds may be screened for specific binding to NAAP. In various embodiments, 1, 2, 3, 4, 5, 10, 20, 50, 100, or 200 test compounds can be screened for specific binding to NAAP. Examples of test compounds can include antibodies, anticalins, oligonucleotides, proteins (e.g., ligands or receptors), or small molecules.

In related embodiments, variants of NAAP can be used to screen for binding of test compounds, such as antibodies, to NAAP, a variant of NAAP, or a combination of NAAP and/or one or more variants NAAP. In an embodiment, a variant of NAAP can be used to screen for compounds that bind to a variant of NAAP, but not to NAAP having the exact sequence of a sequence of SEQ ID NO:1-58. NAAP variants used to perform such screening can have a range of about 50% to about 99% sequence identity to NAAP, with various embodiments having 60%, 70%, 75%, 80%, 85%, 90%, and 95% sequence identity.

In an embodiment, a compound identified in a screen for specific binding to NAAP can be closely related to the natural ligand of NAAP, e.g., a ligand or fragment thereof, a natural substrate, a structural or functional miinetic, or a natural binding partner (Coligan, J. E. et al. (1991) Current Protocols in Immunolor 1(2):Chapter 5). In another embodiment, the compound thus identified can be a natural ligand of a receptor NAAP (Howard, A. D. et al. (2001) Trends Pharmacol. Sci.22:132-140; Wise, A. et al. (2002) Drug Discovery Today 7:235-246).

In other embodiments, a compound identified in a screen for specific binding to NAAP can be closely related to the natural receptor to which NAAP binds, at least a fragment of the receptor, or a fragment of the receptor including all or a portion of the ligand binding site or binding pocket. For example, the compound may be a receptor for NAAP which is capable of propagating a signal, or a decoy receptor for NAAP which is not capable of propagating a signal (Ashkenazi, A. and V. M. Divit (1999) Curr. Opin. Cell Biol. 11:255-260; Mantovani, A. et al. (2001) Trends Imunol. 22:328-336). The compound can be rationally designed using known techniques. Examples of such techniques include those used to construct the compound etanercept (ENBREL; Amgen Inc., Thousand Oaks Calif.), which is efficacious for treating rheumatoid arthritis in humans. Etanercept is an engineered p75 tumor necrosis factor (TNF) receptor dimer linked to the Fc portion of human IgG₁ (Taylor, P. C. et al. (2001) Curr. Opin. Immunol. 13:611-616).

In one embodiment, two or more antibodies having similar or, alternatively, different specificities can be screened for specific binding to NAAP, fragments of NAAP, or variants of NAAP. The binding specificity of the antibodies thus screened can thereby be selected to identify particular fragments or variants of NAAP. In one embodiment, an antibody can be selected such that its binding specificity allows for preferential identification of specific fragments or variants of NAAP. In another embodiment, an antibody can be selected such that its binding specificity allows for preferential diagnosis of a specific disease or condition having increased, decreased, or otherwise abnormal production of NAAP.

In an embodiment, anticalins can be screened for specific binding to NAAP, fragments of NAAP, or variants of NAAP. Anticalins are ligand-binding proteins that have been constructed based on a lipocalin scaffold (Weiss, G. A. and H. B. Lowman (2000) Chem. Biol. 7:R177-R184; Skerra, A. (2001) J. Biotechnol. 74:257-275). The protein architecture of lipocalins can include a beta-barrel having eight antiparallel beta-strands, which supports four loops at its open end. These loops form the natural ligand-binding site of the lipocalins, a site which can be re-engineered in vitro by amino acid substitutions to impart novel binding specificities. The amino acid substitutions can be made using methods known in the art or described herein, and can include conservative substitutions (e.g., substitutions that do not alter binding specificity) or substitutions that modestly, moderately, or significantly alter binding specificity.

In one embodiment, screening for compounds which specifically bind to, stimulate, or inhibit NAAP involves producing appropriate cells which express NAAP, either as a secreted protein or on the cell membrane. Preferred cells can include cells from mammals, yeast, Drosophila, or E. coli. Cells expressing NAAP or cell membrane fractions which contain NAAP are then contacted with a test compound and binding, stimulation, or inhibition of activity of either NAAP or the compound is analyzed.

An assay may simply test binding of a test compound to the polypeptide, wherein binding is detected by a fluorophore, radioisotope, enzyme conjugate, or other detectable label. For example, the assay may comprise the steps of combining at least one test compound with NAAP, either in solution or affixed to a solid support, and detecting the binding of NAAP to the compound. Alternatively, the assay may detect or measure binding of a test compound in the presence of a labeled competitor. Additionally, the assay may be carried out using cell-free preparations, chemical libraries, or natural product mixtures, and the test compound(s) may be free in solution or affixed to a solid support.

An assay can be used to assess the ability of a compound to bind to its natural ligand and/or to inhibit the binding of its natural ligand to its natural receptors. Examples of such assays include radio-labeling assays such as those described in U.S. Pat. No. 5,914,236 and U.S. Pat. No. 6,372,724. In a related embodiment, one or more amino acid substitutions can be introduced into a polypeptide compound (such as a receptor) to improve or alter its ability to bind to its natural ligands (Matthews, D. J. and J. A. Wells. (1994) Chem. Biol. 1:25-30). In another related embodiment, one or more amino acid substitutions can be introduced into a polypeptide compound (such as a ligand) to improve or alter its ability to bind to its natural receptors (Cunningham, B. C. and J. A. Wells (1991) Proc. Natl. Acad. Sci. USA 88:3407-3411; Lowman, H. B. et al. (1991) J. Biol. Chem. 266:10982-10988).

NAAP, fragments of NAAP, or variants of NAAP may be used to screen for compounds 1o that modulate the activity of NAAP. Such compounds may include agonists, antagonists, or partial or inverse agonists. In one embodiment, an assay is performed under conditions permissive for NAAP activity, wherein NAAP is combined with at least one test compound, and the activity of NAAP in the presence of a test compound is compared with the activity of NAAP in the absence of the test compound. A change in the activity of NAAP in the presence of the test compound is indicative of a compound that modulates the activity of NAAP. Alternatively, a test compound is combined with an in vitro or cell-free system comprising NAAP under conditions suitable for NAAP activity, and the assay is performed. In either of these assays, a test compound which modulates the activity of NAAP may do so indirectly and need not come in direct contact with the test compound. At least one and up to a plurality of test compounds may be screened.

In another embodiment, polynucleotides encoding NAAP or their mammalian homologs may be “knocked out” in an animal model system using homologous recombination in embryonic stem (ES) cells. Such techniques are well known in the art and are useful for the generation of animal models of human disease (see, e.g., U.S. Pat. No. 5,175,383 and U.S. Pat. No. 5,767,337). For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (neo; Capecchi, M. R. (1989) Science 244:1288-1292). The vector integrates into the corresponding region of the host genome by homologous recombination. Alternatively, homologous recombination takes place using the Cre-loxP system to knockout a gene of interest in a tissue- or developmental stage-specific manner (Marth, J. D. (1996) Clin. Invest. 97:1999-2002; Wagner, K. U. et al. (1997) Nucleic Acids Res. 25:4323-4330). Transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Transgenic animals thus generated may be tested with potential therapeutic or toxic agents.

Polynucleotides encoding NAAP may also be manipulated in vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al. (1998) Science 282:1145-1147).

Polynucleotides encoding NAAP can also be used to create “knockin” humanized animals (pigs) or transgenic animals (mice or rats) to model human disease. With knockin technology, a region of a polynucleotide encoding NAAP is injected into animal ES cells, and the injected sequence integrates into the animal cell genome. Transformed cells are injected into blastulae, and the blastulae are implanted as described above. Transgenic progeny or inbred lines are studied and treated with potential pharmaceutical agents to obtain information on treatment of a human disease. Alternatively, a mammal inbred to overexpress NAAP, e.g., by secreting NAAP in its milk, may also serve as a convenient source of that protein (Janne, J. et al. (1998) Biotechnol. Annu. Rev. 4:55-74).

Therapeutics

Chemical and structural similarity, e.g., in the context of sequences and motifs, exists between regions of NAAP and nucleic acid-associated proteins. In addition, examples of tissues expressing NAAP can be found in Table 6 and can also be found in Example XL. Therefore, NAAP appears to play a role in cell proliferative, neurological, developmental, and autoimmune/inflammatory disorders, and infections. In the treatment of disorders associated with increased NAAP expression or activity, it is desirable to decrease the expression or activity of NAAP. In the treatment of disorders associated with decreased NAAP expression or activity, it is desirable to increase the expression or activity of NAAP.

Therefore, in one embodiment, NAAP or a fragment or derivative thereof may be administered to a subject to treat or prevent a disorder associated with decreased expression or activity of NAAP. Examples of such disorders include, but are not limited to, a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, a cancer of the adrenal gland, bladder, bone, bone marrow, brain, breast, cervix, colon, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorder of the central nervous system, cerebral palsy, a neuroskeletal disorder, an autonomic nervous system disorder, a cranial nerve disorder, a spinal cord disease, muscular dystrophy and other neuromuscular disorder, a peripheral nervous system disorder, dermatomyositis and polymyositis, inherited, metabolic, endocrine, and toxic myopathy, myasthenia gravis, periodic paralysis, a mental disorder including mood, anxiety, and schizophrenic disorder, seasonal affective disorder (SAD), akathesia, amnesia, catatonia, diabetic neuropathy, tardive dyskinesia, dystonias, paranoid psychoses, postherpetic neuralgia, and Tourette's disorder; a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjogren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; and an infection, such as those caused by a viral agent classified as adenovirus, arenavirus, bunyavirus, calicivirus, coronavirus, filovirus, hepadnavirus, herpesvirus, flavivirus, orthomyxovirus, parvovirus, papovavirus, paramyxovirus, picornavirus, poxvirus, reovirus, retrovirus, rhabdovirus, or togavirus; an infection caused by a bacterial agent classified as pneumococcus, staphylococcus, streptococcus, bacillus, corynebacterium, clostridium, meningococcus, gonococcus, listeria, moraxella, kingella, haemophilus, legionella, bordetella, gram-negative enterobacterium including shigella, salmonella, or campylobacter, pseudomonas, vibrio, brucella, francisella, yersinia, bartonella, norcardium, actinomyces, mycobacterium, spirochaetale, rickettsia, chlamydia, or mycoplasma; an infection caused by a fungal agent classified as aspergillus, blastomyces, dermatophytes, cryptococcus, coccidioides, malasezzia, histoplasma, or other mycosis-causing fungal agent; and an infection caused by a parasite classified as plasmodium or malaria-causing, parasitic entamoeba, leishmania, trypanosoma, toxoplasma, pneumocystis carinii, intestinal protozoa such as giardia, trichomonas, tissue nematode such as trichinella, intestinal nematode such as ascaris, lymphatic filarial nematode, trematode such as schistosoma, and cestode such as tapeworm.

In another embodiment, a vector capable of expressing NAAP or a fragment or derivative thereof may be administered to a subject to treat or prevent a disorder associated with decreased expression or activity of NAAP including, but not limited to, those described above.

In a further embodiment, a composition comprising a substantially purified NAAP in conjunction with a suitable pharmaceutical carrier may be administered to a subject to treat or prevent a disorder associated with decreased expression or activity of NAAP including, but not limited to, those provided above.

In still another embodiment, an agonist which modulates the activity of NAAP may be administered to a subject to treat or prevent a disorder associated with decreased expression or activity of NAAP including, but not limited to, those listed above.

In a further embodiment, an antagonist of NAAP may be administered to a subject to treat or prevent a disorder associated with increased expression or activity of NAAP. Examples of such disorders include, but are not limited to, those cell proliferative, neurological, developmental, and autoinmuunefinflammatory disorders, and infections described above. In one aspect, an antibody which specifically binds NAAP may be used directly as an antagonist or indirectly as a targeting or delivery mechanism for bringing a pharmaceutical agent to cells or tissues which express NAAP.

In an additional embodiment, a vector expressing the complement of the polynucleotide encoding NAAP may be administered to a subject to treat or prevent a disorder associated with increased expression or activity of NAAP including, but not limited to, those described above.

In other embodiments, any protein, agonist, antagonist, antibody, complementary sequence, or vector embodiments may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of NAAP may be produced using methods which are generally known in the art. In particular, purified NAAP may be used to produce antibodies or to screen libraries of pharmaceutical agents to identify those which specifically bind NAAP. Antibodies to NAAP may also be generated using methods that are well known in the art. Such antibodies may include, but are not limited to, polyclonal, monoclonal, chimeric, and single chain antibodies, Fab fragments, and fragments produced by a Fab expression library. In an embodiment, neutralizing antibodies (i.e., those which inhibit dimer formation) can be used therapeutically. Single chain antibodies (e.g., from camels or llamas) may be potent enzyme inhibitors and may have application in the design of peptide mimetics, and in the development of immuno-adsorbents and biosensors (Muyldermans, S. (2001) J. Biotechnol. 74:277-302).

For the production of antibodies, various hosts including goats, rabbits, rats, mice, camels, dromedaries, llamas, humans, and others may be immunized by injection with NAAP or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to NAAP have an amino acid sequence consisting of at least about 5 amino acids, and generally will consist of at least about 10 amino acids. It is also preferable that these oligopeptides, peptides, or fragments are substantially identical to a portion of the amino acid sequence of the natural protein. Short stretches of NAAP amino acids may be fused with those of another protein, such as KLH, and antibodies to the chimeric molecule may be produced.

Monoclonal antibodies to NAAP may be prepared using any technique which provides for the production of antibody molecules by continuous cell lines in culture. These include, but are not limited to, the hybridoma technique, the human B-cell hybridoma technique, and the EBV-hybridoma technique (Kohler, G. et al. (1975) Nature 256:495-497; Kozbor, D. et al. (1985) J. Immunol. Methods 81:31-42; Cote, R. J. et al. (1983) Proc. Natl. Acad. Sci. USA 80:2026-2030; Cole, S. P. et al. (1984) Mol. Cell Biol. 62:109-120).

In addition, techniques developed for the production of “chimeric antibodies,” such as the splicing of mouse antibody genes to human antibody genes to obtain a molecule with appropriate antigen specificity and biological activity, can be used (Morrison, S. L. et al. (1984) Proc. Natl. Acad. Sci. USA 81:6851-6855; Neuberger, M. S. et al. (1984) Nature 312:604-608; Takeda, S. et al. (1985) Nature 314:452-454). Alternatively, techniques described for the production of single chain antibodies may be adapted, using methods known in the art, to produce NAAP-specific single chain antibodies. Antibodies with related specificity, but of distinct idiotypic composition, may be generated by chain shuffling from random combinatorial immunoglobulin libraries (Burton, D. R. (1991) Proc. Natl. Acad. Sci. USA 88:10134-10137).

Antibodies may also be produced by inducing in vivo production in the lymphocyte population or by screening immunoglobulin libraries or panels of highly specific binding reagents as disclosed in the literature (Orlandi, R. et al. (1989) Proc. Natl. Acad. Sci. USA 86:3833-3837; Winter, G. et al. (1991) Nature 349:293-299).

Antibody fragments which contain specific binding sites for NAAP may also be generated. For example, such fragments include, but are not limited to, F(ab′)₂ fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity (Huse, W. D. et al. (1989) Science 246:1275-1281).

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between NAAP and its specific antibody. A two-site, monoclonal-based immunoassay utilizing monoclonal antibodies reactive to two non-interfering NAAP epitopes is generally used, but a competitive binding assay may also be employed (Pound, supra).

Various methods such as Scatchard analysis in conjunction with radioimmunoassay techniques may be used to assess the affinity of antibodies for NAAP. Affinity is expressed as an association constant, Kₐ, which is defined as the molar concentration of NAAP-antibody complex divided by the molar concentrations of free antigen and free antibody under equilibrium conditions. The Kₐ determined for a preparation of polyclonal antibodies, which are heterogeneous in their affinities for multiple NAAP epitopes, represents the average affinity, or avidity, of the antibodies for NAAP. The Kₐ determined for a preparation of monoclonal antibodies, which are monospecific for a particular NAAP epitope, represents a true measure of affinity. High-affinity antibody preparations with Kₐ ranging from about 10⁹ to 10¹² L/mole are preferred for use in immunoassays in which the NAAP-antibody complex must withstand rigorous manipulations. Low-affinity antibody preparations with Kₐ ranging from about 10⁶ to 10⁷ L/mole are preferred for use in immunopurification and similar procedures which ultimately require dissociation of NAAP, preferably in active form, from the antibody (Catty, D. (1988) Antibodies, Volume I: A Practical Approach, IRL Press, Washington D.C.; Liddell, J. E. and A. Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of NAAP-antibody complexes. Procedures for evaluating antibody specificity, titer, and avidity, and guidelines for antibody quality and usage in various applications, are generally available (Catty, supra; Coligan et al., supra).

In another embodiment of the invention, polynucleotides encoding NAAP, or any fragment or complement thereof, may be used for therapeutic purposes. In one aspect, modifications of gene expression can be achieved by designing complementary sequences or antisense molecules (DNA, RNA, PNA, or modified oligonucleotides) to the coding or regulatory regions of the gene encoding NAAP. Such technology is well known in the art, and antisense oligonucleotides or larger fragments can be designed from various locations along the coding or control regions of sequences encoding NAAP (Agrawal, S., ed. (1996) Antisense Therapeutics, Humana Press, Totawa N.J.).

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein (Slater, J. E. et al. (1998) J. Allergy Clin. Immunol. 102:469-475; Scanlon, K. J. et al. (1995) 9:1288-1296). Antisense sequences can also be introduced intracellularly through the use of viral vectors, such as retrovirus and adeno-associated virus vectors (Miller, A. D. (1990) Blood 76:271; Ausubel et al., supra; Uckert, W. and W. Walther (1994) Pharmacol. Ther. 63:323-347). Other gene delivery mechanisms include liposome-derived systems, artificial viral envelopes, and other systems known in the art (Rossi, J. J. (1995) Br. Med. Bull. 51:217-225; Boado, R. J. et al. (1998) J. Pharm. Sci. 87:1308-1315; Morris, M. C. et al. (1997) Nucleic Acids Res. 25:2730-2736).

In another embodiment of the invention, polynucleotides encoding NAAP may be used for somatic or germline gene therapy. Gene therapy may be performed to (i) correct a genetic deficiency (e.g., in the cases of severe combined immunodeficiency (SCID)-X1 disease characterized by X-linked inheritance (Cavazzana-Calvo, M. et al. (2000) Science 288:669-672), severe combined immunodeficiency syndrome associated with an inherited adenosine deaminase (ADA) deficiency (Blaese, R. M. et al. (1995) Science 270:475-480; Bordignon, C. et al. (1995) Science 270:470-475), cystic fibrosis (Zabner, J. et al. (1993) Cell 75:207-216; Crystal, R. G. et al. (1995) Hum. Gene Therapy 6:643-666; Crystal, R. G. et al. (1995) Hum. Gene Therapy 6:667-703), thalassamias, familial hypercholesteroleria, and hemophilia resulting from Factor VIII or Factor IX deficiencies (Crystal, R. G. (1995) Science 270:404-410; Verma, I. M. and N. Somia (1997) Nature 389:239-242)), (ii) express a conditionally lethal gene product (e.g., in the case of cancers which result from unregulated cell proliferation), or (iii) express a protein which affords protection against intracellular parasites (e.g., against human retroviruses, such as human immunodeficiency virus (HI) (Baltimore, D. (1988) Nature 335:395-396; Poeschla, E. et al. (1996) Proc. Natl. Acad. Sci. USA 93:11395-11399), hepatitis B or C virus (HBV, HCV); fungal parasites, such as Candida albicans and Paracoccidioides brasiliensis; and protozoan parasites such as Plasmodium falciparum and Trypanosoma cruzi). In the case where a genetic deficiency in NAAP expression or regulation causes disease, the expression of NAAP from an appropriate population of transduced cells may alleviate the clinical manifestations caused by the genetic deficiency.

In a further embodiment of the invention, diseases or disorders caused by deficiencies in NAAP are treated by constructing mammalian expression vectors encoding NAAP and introducing these vectors by mechanical means into NAAP-deficient cells. Mechanical transfer technologies for use with cells in vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and W. F. Anderson (1993) Annu. Rev. Biochem. 62:191-217; Ivics, Z. (1997) Cell 91:501-510; Boulay, J.-L. and H. Récipon (1998) Curr. Opin. Biotechnol. 9:445-450).

Expression vectors that may be effective for the expression of NAAP include, but are not limited to, the PCDNA 3.1, EPITAG, PRCCMV2, PREP, PVAX, PCR2-TOPOTA vectors (Invitrogen, Carlsbad Calif.), PCMV-SCRIPT, PCMV-TAG, PEGSH/PERV (Stratagene, La Jolla Calif.), and PTET-OFF, PTET-ON, PTRE2, NTRE2-LUC, PTK-HYG (Clontech, Palo Alto Calif.). NAAP maybe expressed using (i) a constitutively active promoter, (e.g., from cytomegalovirus (CMV), Rous sarcoma virus (RSV), SV40 virus, thymidine kinase (TK), or β-actin genes), (ii) an inducible promoter (e.g., the tetracycline-regulated promoter (Gossen, M. and H. Bujard (1992) Proc. Natl. Acad. Sci. USA 89:5547-5551; Gossen, M. et al. (1995) Science 268:1766-1769; Rossi, F. M. V. and H. M. Blau (1998) Curr. Opin. Biotechnol. 9:451-456), commercially available in the T-REX plasmid (Invitrogen)); the ecdysone-inducible promoter (available in the plasmids PVGRXR and PIND; Invitrogen); the FK506/rapamycin inducible promoter; or the RU486/mifepristone inducible promoter (Rossi, F. M. V. and H. M. Blau, supra)), or (iii) a tissue-specific promoter or the native promoter of the endogenous gene encoding NAAP from a normal individual.

Commercially available liposome transformation kits (e.g., the PERFECT LIPID TRANSFECTION KIT, available from Invitrogen) allow one with ordinary skill in the art to deliver polynucleotides to target cells in culture and require minimal effort to optimize experimental parameters. In the alternative, transformation is performed using the calcium phosphate method (Graham, F. L. and A. J. Eb (1973) Virology 52:456-467), or by electroporation (Neumann, E. et al. (1982) EMBO J. 1:841-845). The introduction of DNA to primary cells requires modification of these standardized mammalian transfection protocols.

In another embodiment of the invention, diseases or disorders caused by genetic defects with respect to NAAP expression are treated by constructing a retrovirus vector consisting of (i) the polynucleotide encoding NAAP under the control of an independent promoter or the retrovirus long terminal repeat (LTR) promoter, (ii) appropriate RNA packaging signals, and (iii) a Rev-responsive element (RRE) along with additional retrovirus cis-acting RNA sequences and coding sequences required for efficient vector propagation. Retrovirus vectors (e.g., PFB and PFBNEO) are commercially available (Stratagene) and are based on published data (Riviere, I. et al. (1995) Proc. Natl. Acad. Sci. USA 92:6733-6737), incorporated by reference herein. The vector is propagated in an appropriate vector producing cell line (VPCL) that expresses an envelope gene with a tropism for receptors on the target cells or a promiscuous envelope protein such as VSVg (Armentano, D. et al. (1987) J. Virol. 61:1647-1650; Bender, M. A. et al. (1987) J. Virol. 61:1639-1646; Adam, M. A. and A. D. Miller (1988) J. Virol. 62:3802-3806; Dull, T. et al. (1998) J. Virol. 72:8463-8471; Zufferey, R. et al. (1998) J. Virol. 72:9873-9880). U.S. Pat. No. 5,910,⁴³⁴ to Rigg (“Method for obtaining retrovinis packaging cell lines producing high transducing efficiency retroviral supernatant”) discloses a method for obtaining retrovirus packaging cell lines and is hereby incorporated by reference. Propagation of retrovirus vectors, transduction of a population of cells (e.g., CD4⁺ T-cells), and the return of transduced cells to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M. L. (1997) J. Virol. 71:4707-4716; Ranga, U. et al. (1998) Proc. Natl. Acad. Sci. USA 95:1201-1206; Su, L. (1997) Blood 89:2283-2290).

In an embodiment, an adenovirus-based gene therapy delivery system is used to deliver polynucleotides encoding NAAP to cells which have one or more genetic abnormalities with respect to the expression of NAAP. The construction and packaging of adenovirus-based vectors are well known to those with ordinary skill in the art. Replication defective adenovirus vectors have proven to be versatile for importing genes encoding immunoregulatory proteins into intact islets in the pancreas (Csete, M. E. et al. (1995) Transplantation 27:263-268). Potentially useful adenoviral vectors are described in U.S. Pat. No. 5,707,618 to Armentano (“Adenovirus vectors for gene therapy”), hereby incorporated by reference. For adenoviral vectors, see also Antinozzi, P. A. et al. (1999; Annu. Rev. Nutr. 19:511-544) and Verma, I. M. and N. Somia (1997; Nature 18:389:239-242).

In another embodiment, a herpes-based, gene therapy delivery system is used to deliver polynucleotides encoding NAAP to target cells which have one or more genetic abnormalities with respect to the expression of NAAP. The use of herpes simplex virus (HSV)-based vectors may be especially valuable for introducing NAAP to cells of the central nervous system, for which HSV has a tropism. The construction and packaging of herpes-based vectors are well known to those with ordinary skill in the art. A replication-competent herpes simplex virus (HSV) type 1-based vector has been used to deliver a reporter gene to the eyes of primates (Liu, X. et al. (1999) Exp. Eye Res. 169:385-395). The construction of a HSV-1 virus vector has also been disclosed in detail in U.S. Pat. No. 5,804,413 to DeLuca (“Herpes simplex virus strains for gene transfer”), which is hereby incorporated by reference. U.S. Pat. No. 5,804,413 teaches the use of recombinant HSV d92 which consists of a genome containing at least one exogenous gene to be transferred to a cell under the control of the appropriate promoter for purposes including human gene therapy. Also taught by this patent are the construction and use of recombinant HSV strains deleted for ICP4, ICP27 and ICP22. For HSV vectors, see also Goins, W. F. et al. (1999; J. Virol. 73:519-532) and Xu, H. et al. (1994; Dev. Biol. 163:152-161). The manipulation of cloned herpesvirus sequences, the generation of recombinant virus following the transfection of multiple plasmids containing different segments of the large herpesvirus genomes, the growth and propagation of herpesvirus, and the infection of cells with herpesvirus are techniques well known to those of ordinary skill in the art.

In another embodiment, an alphavirus (positive, single-stranded RNA virus) vector is used to deliver polynucleotides encoding NAAP to target cells. The biology of the prototypic alphavirus, Semliki Forest Virus (SFV), has been studied extensively and gene transfer vectors have been based on the SFV genome (Garoff, H. and K.-J. Li (1998) Curr. Opin. Biotechnol. 9:464-469). During alphavirus RNA replication, a subgenornic RNA is generated that normally encodes the viral capsid proteins. This subgenomic RNA replicates to higher levels than the full length genoric RNA, resulting in the overproduction of capsid proteins relative to the viral proteins with enzymatic activity (e.g., protease and polymerase). Similarly, inserting the coding sequence for NAAP into the alphavirus genome in place of the capsid-coding region results in the production of a large number of NAAP-coding RNAs and the synthesis of high levels of NAAP in vector transduced cells. While alphavirus infection is typically associated with cell lysis within a few days, the ability to establish a persistent infection in hamster normal kidney cells (BHK-21) with a variant of Sindbis virus (SIN) indicates that the lytic replication of alphaviruses can be altered to suit the needs of the gene therapy application (Dryga, S. A. et al. (1997) Virology 228:74-83). The wide host range of alphaviruses will allow the introduction of NAAP into a variety of cell types. The specific transduction of a subset of cells in a population may require the sorting of cells prior to transduction. The methods of manipulating infectious cDNA clones of alphaviruses, performing alphavirus cDNA and RNA transfections, and performing alphavirus infections, are well known to those with ordinary skill in the art.

Oligonucleotides derived from the transcription initiation site, e.g., between about positions −10 and +10 from the start site, may also be employed to inhibit gene expression. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) in Huber, B. E. and B. I. Carr, Molecular and Immunologic Approaches, Futura Publishing, Mt. Kisco N.Y., pp. 163-177). A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of RNA molecules encoding NAAP.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA molecules encoding NAAP. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3 ′ ends of the molecule, or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

In other embodiments of the invention, the expression of one or more selected polynucleotides of the present invention can be altered, inhibited, decreased, or silenced using RNA interference (RNAi) or post-transcriptional gene silencing (PTGS) methods known in the art. RNAi is a post-transcriptional mode of gene silencing in which double-stranded RNA (dsRNA) introduced into a targeted cell specifically suppresses the expression of the homologous gene (i.e., the gene bearing the sequence complementary to the dsRNA). This effectively knocks out or substantially reduces the expression of the targeted gene. PIGS can also be accomplished by use of DNA or DNA fragments as well. RNAi methods are described by Fire, A. et al. (1998; Nature 391:806-811) and Gura, T. (2000; Nature 404:804-808). PTGS can also be initiated by introduction of a complementary segment of DNA into the selected tissue using gene delivery and/or viral vector delivery methods described herein or known in the art.

RNAi can be induced in mammalian cells by the use of small interfering RNA also known as siRNA. SiRNA are shorter segments of dsRNA (typically about 21 to 23 nucleotides in length) that result in vivo from cleavage of introduced dsRNA by the action of an endogenous ribonuclease. SiRNA appear to be the mediators of the RNAi effect in mammals. The most effective siRNAs appear to be 21 nucleotide dsRNAs with 2 nucleotide 3′ overhangs. The use of siRNA for inducing RNAi in mammalian cells is described by Elbashir, S. M. et al. (2001; Nature 411:494-498).

SiRNA can either be generated indirectly by introduction of dsRNA into the targeted cell, or directly by mammalian transfection methods and agents described herein or known in the art (such as liposome-mediated transfection, viral vector methods, or other polynucleotide delivery/introductory methods). Suitable SiRNAs can be selected by examining a transcript of the target polynucleotide (e.g., mRNA) for nucleotide sequences downstream from the AUG start codon and recording the occurrence of each nucleotide and the 3′ adjacent 19 to 23 nucleotides as potential siRNA target sites, with sequences having a 21 nucleotide length being preferred. Regions to be avoided for target siRNA sites include the 5′ and 3′ untranslated regions (UTRs) and regions near the start codon (within 75 bases), as these may be richer in regulatory protein binding sites. UTR-binding proteins and/or translation initiation complexes may interfere with binding of the siRNP endonuclease complex. The selected target sites for siRNA can then be compared to the appropriate genome database (e.g., human, etc.) using BLAST or other sequence comparison algorithms known in the art. Target sequences with significant homology to other coding sequences can be eliminated from consideration. The selected SiRNAs can be produced by chemical synthesis methods known in the art or by in vitro transcription using commercially available methods and kits such as the SILENCER siRNA construction kit (Ambion, Austin Tex.).

In alternative embodiments, long-term gene silencing and/or RNAi effects can be induced in selected tissue using expression vectors that continuously express siRNA. This can be accomplished using expression vectors that are engineered to express hairpin RNAs (shRNAs) using methods known in the art (see, e.g., Brummelkamp, T. R. et al. (2002) Science 296:550-553; and Paddison, P. J. et al. (2002) Genes Dev. 16:948-958). In these and related embodiments, shRNAs can be delivered to target cells using expression vectors known in the art. An example of a suitable expression vector for delivery of siRNA is the PSILENCER1.0-U6 (circular) plasmid (Ambion). Once delivered to the target tissue, shRNAs are processed in vivo into siRNA-like molecules capable of carrying out gene-specific silencing.

In various embodiments, the expression levels of genes targeted by RNAi or PTGS methods can be determined by assays for mRNA and/or protein analysis. Expression levels of the mRNA of a targeted gene, can be determined by northern analysis methods using, for example, the NORTHERNMAX-GLY kit (Ambion); by microarray methods; by PCR methods; by real time PCR methods; and by other RNA/polynucleotide assays known in the art or described herein. Expression levels of the protein encoded by the targeted gene can be determined by Western analysis using standard techniques known in the art.

An additional embodiment of the invention encompasses a method for screening for a compound which is effective in altering expression of a polynucleotide encoding NAAP. Compounds which may be effective in altering expression of a specific polynucleotide may include, but are not limited to, oligonucleotides, antisense oligonucleotides, triple helix-forming oligonucleotides, transcription factors and other polypeptide transcriptional regulators, and non-macromolecular chemical entities which are capable of interacting with specific polynucleotide sequences. Effective compounds may alter polynucleotide expression by acting as either inhibitors or promoters of polynucleotide expression. Thus, in the treatment of disorders associated with increased NAAP expression or activity, a compound which specifically inhibits expression of the polynucleotide encoding NAAP may be therapeutically useful, and in the treatment of disorders associated with decreased NAAP expression or activity, a compound which specifically promotes expression of the polynucleotide encoding NAAP may be therapeutically useful.

In various embodiments, one or more test compounds may be screened for effectiveness in altering expression of a specific polynucleotide. A test compound may be obtained by any method commonly known in the art, including chemical modification of a compound known to be effective in altering polynucleotide expression; selection from an existing, commercially-available or proprietary library of naturally-occurring or non-natural chemical compounds; rational design of a compound based on chemical and/or structural properties of the target polynucleotide; and selection from a library of chemical compounds created combinatorially or randomly. A sample comprising a polynucleotide encoding NAAP is exposed to at least one test compound thus obtained. The sample may comprise, for example, an intact or permeabilized cell, or an in vitro cell-free or reconstituted biochemical system. Alterations in the expression of a polynucleotide encoding NAAP are assayed by any method commonly known in the art. Typically, the expression of a specific nucleotide is detected by hybridization with a probe having a nucleotide sequence complementary to the sequence of the polynucleotide encoding NAAP. The amount of hybridization may be quantified, thus forming the basis for a comparison of the expression of the polynucleotide both with and without exposure to one or more test compounds. Detection of a change in the expression of a polynucleotide exposed to a test compound indicates that the test compound is effective in altering the expression of the polynucleotide. A screen for a compound effective in altering expression of a specific polynucleotide can be carried out, for example, using a Schizosaccharomyces pombe gene expression system (Atkins, D. et al. (1999) U.S. Pat. No. 5,932,435; Arndt, G. M. et al. (2000) Nucleic Acids Res. 28:E15) or a human cell line such as HeLa cell (Clarke, M. L. et al. (2000) Biochem. Biophys. Res. Commun. 268:8-13). A particular embodiment of the present invention involves screening a combinatorial library of oligonucleotides (such as deoxyribonucleotides, ribonucleotides, peptide nucleic acids, and modified oligonucleotides) for antisense activity against a specific polynucleotide sequence (Bruice, T. W. et al. (1997) U.S. Pat. No. 5,686,242; Bruice, T. W. et al. (2000) U.S. Pat. No. 6,022,691).

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art (Goldman, C. K. et al. (1997) Nat. Biotechnol. 15:462-466).

Any of the therapeutic methods described above may be applied to any subject in need of such therapy, including, for example, mammals such as humans, dogs, cats, cows, horses, rabbits, and monkeys.

An additional embodiment of the invention relates to the administration of a composition which generally comprises an active ingredient formulated with a pharmaceutically acceptable excipient. Excipients may include, for example, sugars, starches, celluloses, gums, and proteins. Various formulations are commonly known and are thoroughly discussed in the latest edition of Remington's Pharmaceutical Sciences (Maack Publishing, Easton Pa.). Such compositions may consist of NAAP, antibodies to NAAP, and mimetics, agonists, antagonists, or inhibitors of NAAP.

In various embodiments, the compositions described herein, such as pharmaceutical compositions, may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means.

Compositions for pulmonary administration may be prepared in liquid or dry powder form. These compositions are generally aerosolized immediately prior to inhalation by the patient. In the case of small molecules (e.g. traditional low molecular weight organic drugs), aerosol delivery of fast-acting formulations is well-known in the art. In the case of macromolecules (e.g. larger peptides and proteins), recent developments in the field of pulmonary delivery via the alveolar region of the lung have enabled the practical delivery of drugs such as insulin to blood circulation (see, e.g., Patton, J. S. et al., U.S. Pat. No. 5,997,848). Pulmonary delivery allows administration without needle injection, and obviates the need for potentially toxic penetration enhancers.

Compositions suitable for use in the invention include compositions wherein the active ingredients are contained in an effective amount to achieve the intended purpose. The determination of an effective dose is well within the capability of those skilled in the art.

Specialized forms of compositions may be prepared for direct intracellular delivery of macromolecules comprising NAAP or fragments thereof. For example, liposome preparations containing a cell-impermeable macromolecule may promote cell fusion and intracellular delivery of the macromolecule. Alternatively, NAAP or a fragment thereof may be joined to a short cationic N-terminal portion from the HIV Tat-1 protein. Fusion proteins thus generated have been found to transduce into the cells of all tissues, including the brain, in a mouse model system (Schwarze, S. R. et al. (1999) Science 285:1569-1572).

For any compound, the therapeutically effective dose can be estimated initially either in cell culture assays, e.g., of neoplastic cells, or in animal models such as mice, rats, rabbits, dogs, monkeys, or pigs. An animal model may also be used to determine the appropriate concentration range and route of administration. Such information can then be used to determine useful doses and routes for administration in humans.

A therapeutically effective dose refers to that amount of active ingredient, for example NAAP or fragments thereof, antibodies of NAAP, and agonists, antagonists or inibitors of NAAP, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of toxic to therapeutic effects is the therapeutic index, which can be expressed as the ID₅₀/ED₅₀ ratio. Compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting compositions may be administered every 3 to 4 days, every week, or biweekly depending on the half-life and clearance rate of the particular formulation.

Normal dosage amounts may vary from about 0.1 μg to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Diagnostics

In another embodiment, antibodies which specifically bind NAAP may be used for the diagnosis of disorders characterized by expression of NAAP, or in assays to monitor patients being treated with NAAP or agonists, antagonists, or inhibitors of NAAP. Antibodies useful for diagnostic purposes may be prepared in the same manner as described above for therapeutics. Diagnostic assays for NAAP include methods which utilize the antibody and a label to detect NAAP in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring NAAP, including ELISAs, RIAs, and FACS, are known in the art and provide a basis for diagnosing altered or abnormal levels of NAAP expression. Normal or standard values for NAAP expression are established by combining body fluids or cell extracts taken from normal mammalian subjects, for example, human subjects, with antibodies to NAAP under conditions suitable for complex formation. The amount of standard complex formation may be quantitated by various methods, such as photometric means. Quantities of NAAP expressed in subject, control, and disease samples from biopsied tissues are compared with the standard values. Deviation between standard and subject values establishes the parameters for diagnosing disease.

In another embodiment of the invention, polynucleotides encoding NAAP may be used for diagnostic purposes. The polynucleotides which may be used include oligonucleotides, complementary RNA and DNA molecules, and PNAs. The polynucleotides may be used to detect and quantify gene expression in biopsied tissues in which expression of NAAP may be correlated with disease. The diagnostic assay may be used to determine absence, presence, and excess expression of NAAP, and to monitor regulation of NAAP levels during therapeutic intervention.

In one aspect, hybridization with PCR probes which are capable of detecting polynucleotides, including genonic sequences, encoding NAAP or closely related molecules may be used to identify nucleic acid sequences which encode NAAP. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding NAAP, allelic variants, or related sequences.

Probes may also be used for the detection of related sequences, and may have at least 50% sequence identity to any of the NAAP encoding sequences. The hybridization probes of the subject invention may be DNA or RNA and may be derived from the sequence of SEQ ID NO:59-116 or from genomic sequences including promoters, enhancers, and introns of the NAAP gene.

Means for producing specific hybridization probes for polynucleotides encoding NAAP include the cloning of polynucleotides encoding NAAP or NAAP derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³⁵S, or by enzymatic labels, such as alkaline phosphatase coupled to the probe via avidin/biotin coupling systems, and the like.

Polynucleotides encoding NAAP may be used for the diagnosis of disorders associated with expression of NAAP. Examples of such disorders include, but are not limited to, a cell proliferative disorder such as actinic keratosis, arteriosclerosis, atherosclerosis, bursitis, cirrhosis, hepatitis, mixed connective tissue disease (MCID)), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, a cancer of the adrenal gland, bladder, bone, bone marrow, brain, breast, cervix, colon, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorder of the central nervous system, cerebral palsy, a neuroskeletal disorder, an autonomic nervous system disorder, a cranial nerve disorder, a spinal cord disease, muscular dystrophy and other neuromuscular disorder, a peripheral nervous system disorder, dermatomyositis and polymyositis, inherited, metabolic, endocrine, and toxic myopathy, myasthenia gravis, periodic paralysis, a mental disorder including mood, anniety, and schizophrenic disorder, seasonal affective disorder (SAD), akathesia, amnesia, catatonia, diabetic neuropathy, tardive dyskinesia, dystonias, paranoid psychoses, postherpetic neuralgia, and Tourette's disorder; a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss; an autoimmune/inflammatory disorder such as acquired immunodeficiency syndrome (ADDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthitis, scleroderma, Sjogren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helninthic infections, and trauma; and an infection, such as those caused by a viral agent classified as adenovirus, arenavirus, bunyavirus, calicivirus, coronavirus, filovirus, hepadnavirus, herpesvirus, flavivirus, orthomyxovirus, parvovirus, papovavirus, paramyxovirus, picornavirus, poxvirus, reovirus, retrovirus, rhabdovirus, or togavirus; an infection caused by a bacterial agent classified as pneumococcus, staphylococcus, streptococcus, bacillus, corynebacterium, clostridium, meningococcus, gonococcus, listeria, moraxella, kingella, haemophilus, legionella, bordetella, gram-negative enterobacterium including shigella, salmonella, or campylobacter, pseudomonas, vibrio, brucella, francisella, yersinia, bartonella, norcardium, actinomyces, mycobacterium, spirochaetale, rickettsia, chlamydia, or mycoplasma; an infection caused by a fungal agent classified as aspergillus, blastomyces, dermatophytes, cryptococcus, coccidioides, malasezzia, histoplasma, or other mycosis-causing fungal agent; and an infection caused by a parasite classified as plasmodium or malaria-causing, parasitic entamoeba, leishmania, trypanosoma, toxoplasma, pneumocystis carinii, intestinal protozoa such as giardia, trichomonas, tissue nematode such as trichinella, intestinal nematode such as ascaris, lymphatic filarial nematode, trematode such as schistosoma, and cestode such as tapeworm. Polynucleotides encoding NAAP may be used in Southern or northern analysis, dot blot, or other membrane-based technologies; in PCR technologies; in dipstick, pin, and multiformat ELISA-like assays; and in microarrays utilizing fluids or tissues from patients to detect altered NAAP expression. Such qualitative or quantitative methods are well known in the art.

In a particular embodiment, polynucleotides encoding NAAP may be used in assays that detect the presence of associated disorders, particularly those mentioned above. Polynucleotides complementary to sequences encoding NAAP may be labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of polynucleotides encoding NAAP in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of NAAP, a normal or standard profile for expression is established. This may be accomplished by combining body fluids or cell extracts taken from normal subjects, either animal or human, with a sequence, or a fragment thereof, encoding NAAP, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier, thereby preventing the development or further progression of the cancer.

Additional diagnostic uses for oligonucleotides designed from the sequences encoding NAAP may involve the use of PCR. These oligomers may be chemically synthesized, generated enzymatically, or produced in vitro. Oligomers will preferably contain a fragment of a polynucleotide encoding NAAP, or a fragment of a polynucleotide complementary to the polynucleotide encoding NAAP, and will be employed under optimized conditions for identification of a specific gene or condition. Oligomers may also be employed under less stringent conditions for detection or quantification of closely related DNA or RNA sequences.

In a particular aspect, oligonucleotide primers derived from polynucleotides encoding NAAP may be used to detect single nucleotide polymorphisms (SNPs). SNPs are substitutions, insertions and deletions that are a frequent cause of inherited or acquired genetic disease in humans. Methods of SNP detection include, but are not limited to, single-stranded conformation polymorphism (SSCP) and fluorescent SSCP (fSSCP) methods. In SSCP, oligonucleotide primers derived from polynucleotides encoding NAAP are used to amplify DNA using the polymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In fSCCP, the oligonucleotide primers are fluorescently labeled, which allows detection of the amplimers in high-throughput equipment such as DNA sequencing machines. Additionally, sequence database analysis methods, termed in silico SNP (isSNP), are capable of identifying polymorphisms by comparing the sequence of individual overlapping DNA fragments which assemble into a common consensus sequence. These computer-based methods filter out sequence variations due to laboratory preparation of DNA and sequencing errors using statistical models and automated analyses of DNA sequence chromatograms. In the alternative, SNPs may be detected and characterized by mass spectrometry using, for example, the high throughput MASSARRAY system (Sequenom, Inc., San Diego Calif.).

SNPs may be used to study the genetic basis of human disease. For example, at least 16 common SNPs have been associated with non-insulin-dependent diabetes mellitus. SNPs are also useful for examining differences in disease outcomes in monogenic disorders, such as cystic fibrosis, sickle cell anemia, or chronic granulomatous disease. For example, variants in the mannose-binding lectin, MBL2, have been shown to be correlated with deleterious pulmonary outcomes in cystic fibrosis. SNPs also have utility in pharmacogenomics, the identification of genetic variants that influence a patient's response to a drug, such as life-threatening toxicity. For example, a variation in N-acetyl transferase is associated with a high incidence of peripheral neuropathy in response to the anti-tuberculosis drug isoniazid, while a variation in the core promoter of the ALOX5 gene results in diminished clinical response to treatment with an anti-asthma drug that targets the 5-lipoxygenase pathway. Analysis of the distribution of SNPs in different populations is useful for investigating genetic drift, mutation, recombination, and selection, as well as for tracing the origins of populations and their migrations (Taylor, J. G. et al. (2001) Trends Mol. Med. 7:507-512; Kwok, P.-Y. and Z. Gu (1999) Mol. Med. Today 5:538-543; Nowotny, P. et al. (2001) Curr. Opin. Neurobiol. 11:637-641).

Methods which may also be used to quantify the expression of NAAP include radiolabeling or biotinylating nucleotides, coamplification of a control nucleic acid, and interpolating results from standard curves (Melby, P. C. et al. (1993) J. Immunol. Methods 159:235-244; Duplaa, C. et al. (1993) Anal. Biochem. 212:229-236). The speed of quantitation of multiple samples maybe accelerated by running the assay in a high-throughput format where the oligomer or polynucleotide of interest is presented in various dilutions and a spectrophotometric or colorimetric response gives rapid quantitation.

In further embodiments, oligonucleotides or longer fragments derived from any of the polynucleotides described herein may be used as elements on a microarray. The microarray can be used in transcript imaging techniques which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents which are highly effective and display the fewest side effects may be selected for a patient based on his/her pharmacogenomic profile.

In another embodiment, NAAP, fragments of NAAP, or antibodies specific for NAAP may be used as elements on a microarray. The microartay may be used to monitor or measure protein-protein interactions, drug-target interactions, and gene expression profiles, as described above.

A particular embodiment relates to the use of the polynucleotides of the present invention to generate a transcript image of a tissue or cell type. A transcript image represents the global pattern of gene expression by a particular tissue or cell type. Global gene expression patterns are analyzed by quantifing the number of expressed genes and their relative abundance under given conditions and at a given time (Seilhamer et al., “Comparative Gene Transcript Analysis,” U.S. Pat. No. 5,840,484; hereby expressly incorporated by reference herein). Thus a transcript image may be generated by hybridizing the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity.

Transcript images may be generated using transcripts isolated from tissues, cell lines, biopsies, or other biological samples. The transcript image may thus reflect gene expression in vivo, as in the case of a tissue or biopsy sample, or in vitro, as in the case of a cell line.

Transcript images which profile the expression of the polynucleotides of the present invention may also be used in conjunction with in vitro model systems and preclinical evaluation of pharmaceuticals, as well as toxicological testing of industrial and naturally-occurring environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and N. L. Anderson (2000) Toxicol. Lett. 112-113:467-471). If a test compound has a signature similar to that of a compound with known toxicity, it is likely to share those toxic properties. These fingerprints or signatures are most useful and refined when they contain expression information from a large number of genes and gene families. Ideally, a genome-wide measurement of expression provides the highest quality signature. Even genes whose expression is not altered by any tested compounds are important as well, as the levels of expression of these genes are used to normalize the rest of the expression data. The normalization procedure is useful for comparison of expression data after treatment with different compounds. While the assignment of gene function to elements of a toxicant signature aids in interpretation of toxicity mechanisms, knowledge of gene function is not necessary for the statistical matching of signatures which leads to prediction of toxicity (see, for example, Press Release 00-02 from the National Institute of Environmental Health Sciences, released Feb. 29, 2000, available at http://www.niehs.nih.gov/oc/news/toxchip.htm). Therefore, it is important and desirable in toxicological screening using toxicant signatures to include all expressed gene sequences.

In an embodiment, the toxicity of a test compound can be assessed by treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention may be quantified. The transcript levels in the treated biological sample are compared with levels in an untreated biological sample. Differences in the transcript levels between the two samples are indicative of a toxic response caused by the test compound in the treated sample.

Another embodiment relates to the use of the polypeptides disclosed herein to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifying the number of expressed proteins and their relative abundance under given conditions and at a given time. A profile of a cell's proteome may thus be generated by separating and analyzing the polypeptides of a particular tissue or cell type. In one embodiment, the separation is achieved using two-dimensional gel electrophoresis, in which proteins from a sample are separated by isoelectric focusing in the first dimension, and then according to molecular weight by sodium dodecyl sulfate slab gel electrophoresis in the second dimension (Steiner and Anderson, supra). The proteins are visualized in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment. The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of interest. In some cases, further sequence data may be obtained for definitive protein identification.

A proteomic profile may also be generated using antibodies specific for NAAP to quantify the levels of NAAP expression. In one embodiment, the antibodies are used as elements on a microarray, and protein expression levels are quantified by exposing the microarray to the sample and detecting the levels of protein bound to each array element (Lueking, A. et al. (1999) Anal. Biochem. 270:103-111; Mendoze, L. G. et al. (1999) Biotechniques 27:778-788). Detection maybe performed by a variety of methods known in the art, for example, by reacting the proteins in the sample with a thiol- or amino-reactive fluorescent compound and detecting the amount of fluorescence bound at each array element.

Toxicant signatures at the proteome level are also useful for toxicological screening, and should be analyzed in parallel with toxicant signatures at the transcript level. There is a poor correlation between transcript and protein abundances for some proteins in some tissues (Anderson, N. L. and J. Seilhamer (1997) Electrophoresis 18:533-537), so proteome toxicant signatures may be useful in the analysis of compounds which do not significantly affect the transcript image, but which alter the proteomic profile. In addition, the analysis of transcripts in body fluids is difficult, due to rapid degradation of mRNA, so proteomic profiling may be more reliable and informative in such cases.

In another embodiment, the toxicity of a test compound is assessed by treating a biological sample containing proteins with the test compound. Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the polypeptides of the present invention.

In another embodiment, the toxicity of a test compound is assessed by treating a biological sample containing proteins with the test compound. Proteins from the biological sample are incubated with antibodies specific to the polypeptides of the present invention. The amount of protein recognized by the antibodies is quantified. The amount of protein in the treated biological sample is compared with the amount in an untreated biological sample. A difference in the amount of protein between the two samples is indicative of a toxic response to the test compound in the treated sample.

Microarrays may be prepared, used, and analyzed using methods known in the art (Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, P. et al. (1995) PCT application WO95/35505; Heller, R. A. et al. (1997) Proc. Natl. Acad. Sci. USA 94:2150-2155; Heller, M. J. et al. (1997) U.S. Pat. No. 5,605,662). Various types of microarrays are well known and thoroughly described in Schena, M., ed. (1999; DNA Microarrays: A Practical Approach, Oxford University Press, London).

In another embodiment of the invention, nucleic acid sequences encoding NAAP may be used to generate hybridization probes useful in mapping the naturally occurring genoric sequence. Either coding or noncoding sequences may be used, and in some instances, noncoding sequences may be preferable over coding sequences. For example, conservation of a coding sequence among members of a multi-gene family may potentially cause undesired cross hybridization during chromosomal mapping. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries (Harrington, J. J. et al. (1997) Nat. Genet. 15:345-355; Price, C. M. (1993) Blood Rev. 7:127-134; Trask, B. J. (1991) Trends Genet. 7:149-154). Once mapped, the nucleic acid sequences may be used to develop genetic linkage maps, for example, which correlate the inheritance of a disease state with the inheritance of a particular chromosome region or restriction fragment length polymorphism (RFLP) (Lander, E. S. and D. Botstein (1986) Proc. Natl. Acad. Sci. USA 83:7353-7357).

Fluorescent in situ hybridization (FISH) may be correlated with other physical and genetic map data (Heinz-Ulrich, et al. (1995) in Meyers, supra, pp. 965-968). Examples of genetic map data can be found in various scientific journals or at the Online Mendelian Inheritance in Man (OMIM) World Wide Web site. Correlation between the location of the gene encoding NAAP on a physical map and a specific disorder, or a predisposition to a specific disorder, may help define the region of DNA associated with that disorder and thus may further positional cloning efforts.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the exact chromosomal locus is not known. This information is valuable to investigators searching for disease genes using positional cloning or other gene discovery techniques. Once the gene or genes responsible for a disease or syndrome have been crudely localized by genetic linkage to a particular genomic region, e.g., ataxia-telangiectasia to 11q22-23, any sequences mapping to that area may represent associated or regulatory genes for further investigation (Gatti, R. A. et al. (1988) Nature 336:577-580). The nucleotide sequence of the instant invention may also be used to detect differences in the chromosomal location due to translocation, inversion, etc., among normal, carrier, or affected individuals.

In another embodiment of the invention, NAAP, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between NAAP and the agent being tested may be measured.

Another technique for drug screening provides for high throughput screening of compounds having suitable binding affinity to the protein of interest (Geysen, et al. (1984) PCT application WO84/03564). In this method, large numbers of different small test compounds are synthesized on a solid substrate. The test compounds are reacted with NAAP, or fragments thereof, and washed. Bound NAAP is then detected by methods well known in the art. Purified NAAP can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding NAAP specifically compete with a test compound for binding NAAP. In this manner, antibodies can be used to detect the presence of any peptide which shares one or more antigenic determinants with NAAP.

In additional embodiments, the nucleotide sequences which encode NAAP may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent. The following embodiments are, therefore, to be construed as merely illustrative, and not limitative of the remainder of the disclosure in any way whatsoever.

The disclosures of all patents, applications, and publications mentioned above and below, including U.S. Ser. No. 60/348,442, U.S. Ser. No. 60/337,535, U.S. Ser. No. 60/335,544, U.S. Ser. No. 60/344,650, and U.S. Ser. No. 60/334,762, are hereby expressly incorporated by reference.

### EXAMPLES

I. Construction of cDNA Libraries

Incyte cDNAs were derived from cDNA libraries described in the LIFESEQ GOLD database (Incyte Genomics, Palo Alto Calif.). Some tissues were homogenized and lysed in guanidinium isothiocyanate, while others were homogenized and lysed in phenol or in a suitable mixture of denaturants, such as TRIZOL (Invitrogen), a monophasic solution of phenol and guanidine isothiocyanate. The resulting lysates were centrifuged over CsCl cushions or extracted with chloroform. RNA was precipitated from the lysates with either isopropanol or sodium acetate and ethanol, or by other routine methods.

Phenol extraction and precipitation of RNA were repeated as necessary to increase RNA purity. In some cases, RNA was treated with DNase. For most libraries, poly(A)+ RNA was isolated using oligo d(T)-coupled paramagnetic particles (Promega), OLIGOTEX latex particles (QIAGEN, Chatsworth Calif.), or an OLIGOTEX mRNA purification kit (QIAGEN). Alternatively, RNA was isolated directly from tissue lysates using other RNA isolation kits, e.g., the POLY(A)PURE mRNA purification kit (Ambion, Austin Tex.).

In some cases, Stratagene was provided with RNA and constructed the corresponding cDNA libraries. Otherwise, cDNA was synthesized and cDNA libraries were constructed with the UNIZAP vector system (Stratagene) or SUPERSCRWET plasmid system (Invitrogen), using the recommended procedures or similar methods known in the art (Ausubel et al., supra, ch. 5). Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (Amersham Biosciences) or preparative agarose gel electrophoresis. cDNAs were ligated into compatible restriction enzyme sites of the polylinker of a suitable plasmid, e.g., PBLUESCRIPT plasmid (Stratagene), PSPORT1 plasmid (Invitrogen, Carlsbad Calif.), PCDNA2.1 plasmid (Invitrogen), PBK-CMV plasmid (Stratagene), PCR2-TOPOTA plasmid (Invitrogen), PCMV-ICIS plasmid (Stratagene), pIGEN (Incyte Genomics, Palo Alto Calif.), pRARE (Incyte Genomics), or pINCY (Incyte Genomics), or derivatives thereof. Recombinant plasmids were transformed into competent E. coli cells including XL1-Blue, XL1-BlueMRF, or SOLR from Stratagene or DH5α, DH10B, or ElectroMAX DH10B from Invitrogen.

II. Isolation of cDNA Clones

Plasmids obtained as described in Example I were recovered from host cells by in vivo excision using the UNIZAP vector system (Stratagene) or by cell lysis. Plasmids were purified using at least one of the following: a Magic or WIZARD Minipreps DNA purification system (Promega); an AGTC Miniprep purification kit (Edge Biosystems, Gaithersburg Md.); and QIAWELL 8 Plasmid, QIAWELL 8 Plus Plasmid, QIAWELL 8 Ultra Plasmid purification systems or the R.E.A.L. PREP 96 plasmid purification kit from QIAGEN. Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao, V. B. (1994) Anal. Biochem. 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes, Eugene Oreg.) and a FLUOROSKAN II fluorescence scanner (Labsystems Oy, Helsinki, Finland).

III. Sequencing and Analysis

Incyte cDNA recovered in plasmids as described in Example 11 were sequenced as follows. Sequencing reactions were processed using standard methods or high-throughput instrumentation such as the ABI CATALYST 800 (Applied Biosystems) thermal cycler or the PTC-200 thermal cycler (MJ Research) in conjunction with the HYDRA microdispenser (Robbins Scientific) or the MICROLAB 2200 (Hamilton) liquid transfer system. cDNA sequencing reactions were prepared using reagents provided by Amersham Biosciences or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides were carried out using the MEGABACE 1000 DNA sequencing system (Amersham Biosciences); the ABI PRISM 373 or 377 sequencing system (Applied Biosystems) in conjunction with standard ABI protocols and base calling software; or other sequence analysis systems known in the art. Reading frames within the cDNA sequences were identified using standard methods (Ausubel et al., supra, ch. 7). Some of the cDNA sequences were selected for extension using the techniques disclosed in Example VIII.

The polynucleotide sequences derived from Incyte cDNAs were validated by removing vector, linker, and poly(A) sequences and by masking ambiguous bases, using algorithms and programs based on BLAST, dynamic programming, and dinucleotide nearest neighbor analysis. The Incyte cDNA sequences or translations thereof were then queried against a selection of public databases such as the GenBank primate, rodent, mammalian, vertebrate, and eukaryote databases, and BLOCKS, PRINTS, DOMO, PRODOM; PROTEOME databases with sequences from Homo sapiens, Rattus norvegicus, Mus musculus, Caenorhabditis elegans, Saccharomyces cerevisiae, Schizosaccharomyces pombe, and Candida albicans (Incyte Genomics, Palo Alto Calif.); hidden Markov model (HMM)-based protein family databases such as PFAM, INCY, and TIGRFAM (Haft, D. H. et al. (2001) Nucleic Acids Res. 29:41-43); and HMM-based protein domain databases such as SMART (Schultz, J. et al. (1998) Proc. Natl. Acad. Sci. USA 95:5857-5864; Letunic, I. et al. (2002) Nucleic Acids Res. 30:242-244). (HMM is a probabilistic approach which analyzes consensus primary structures of gene families; see, for example, Eddy, S. R. (1996) Curr. Opin. Struct. Biol. 6:361-365.) The queries were performed using programs based on BLAST, FASTA, BLIMPS, and HMMER. The Incyte cDNA sequences were assembled to produce full length polynucleotide sequences. Alternatively, GenBank cDNAs, GenBank ESTs, stitched sequences, stretched sequences, or Genscan-predicted coding sequences (see Examples IV and V) were used to extend Incyte cDNA assemblages to full length. Assembly was performed using programs based on Phred, Phrap, and Consed, and cDNA assemblages were screened for open reading frames using programs based on GeneMark, BLAST, and FASTA. The full length polynucleotide sequences were translated to derive the corresponding full length polypeptide sequences. Alternatively, a polypeptide may begin at any of the methionine residues of the full length translated polypeptide. Full length polypeptide sequences were subsequently analyzed by querying against databases such as the GenBank protein databases (genpept), SwissProt, the PROTEOME databases, BLOCKS, PRINTS, DOMO, PRODOM, Prosite, hidden Markov model (HMM)-based protein family databases such as PFAM, INCY, and TIGRFAM; and HMM-based protein domain databases such as SMART. Full length polynucleotide sequences are also analyzed using MACDNASIS PRO software (MiraiBio, Alameda Calif.) and LASERGENE software (DNASTAR). Polynucleotide and polypeptide sequence alignments are generated using default parameters specified by the CLUSTAL algorithm as incorporated into the MEGALIGN multisequence alignment program (DNASTAR), which also calculates the percent identity between aligned sequences.

Table 7 summarizes the tools, programs, and algorithms used for the analysis and assembly of Incyte cDNA and full length sequences and provides applicable descriptions, references, and threshold parameters. The first column of Table 7 shows the tools, programs, and algorithms used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score or the lower the probability value, the greater the identity between two sequences).

The programs described above for the assembly and analysis of full length polynucleotide and polypeptide sequences were also used to identify polynucleotide sequence fragments from SEQ ID NO:59-116. Fragments from about 20 to about 4000 nucleotides which are useful in hybridization and amplification technologies are described in Table 4, column 2.

IV. Identification and Editing of Coding Sequences from Genornic DNA

Putative nucleic acid-associated proteins were initially identified by running the Genscan gene identification program against public genomic sequence databases (e.g., gbpri and gbhtg). Genscan is a general-purpose gene identification program which analyzes genomic DNA sequences from a variety of organisms (Burge, C. and S. Karlin (1997) J. Mol. Biol. 268:78-94; Burge, C. and S. Karlin (1998) Curr. Opin. Struct. Biol. 8:346-354). The program concatenates predicted exons to form an assembled cDNA sequence extending from a methionine to a stop codon. The output of Genscan is a FASTA database of polynucleotide and polypeptide sequences. The maximum range of sequence for Genscan to analyze at once was set to 30 kb. To determine which of these Genscan predicted cDNA sequences encode nucleic acid-associated proteins, the encoded polypeptides were analyzed by querying against PFAM models for nucleic acid-associated proteins. Potential nucleic acid-associated proteins were also identified by homology to Incyte cDNA sequences that had been annotated as nucleic acid-associated proteins. These selected Genscan-predicted sequences were then compared by BLAST analysis to the genpept and gbpri public databases. Where necessary, the Genscan-predicted sequences were then edited by comparison to the top BLAST hit from genpept to correct errors in the sequence predicted by Genscan, such as extra or omitted exons. BLAST analysis was also used to find any Incyte cDNA or public cDNA coverage of the Genscan-predicted sequences, thus providing evidence for transcription. When Incyte cDNA coverage was available, this information was used to correct or confirm the Genscan predicted sequence. Full length polynucleotide sequences were obtained by assembling Genscan-predicted coding sequences with Incyte cDNA sequences and/or public cDNA sequences using the assembly process described in Example III. Alternatively, full length polynucleotide sequences were derived entirely from edited or unedited Genscan-predicted coding sequences.

V. Assembly of Genomic Sequence Data with cDNA Sequence Data “Stitched” Sequences

Partial cDNA sequences were extended with exons predicted by the Genscan gene identification program described in Example IV. Partial cDNAs assembled as described in Example m were mapped to genomic DNA and parsed into clusters containing related cDNAs and Genscan exon predictions from one or more genomic sequences. Each cluster was analyzed using an algorithm based on graph theory and dynamic programming to integrate cDNA and genomic information, generating possible splice variants that were subsequently confirmed, edited, or extended to create a full length sequence. Sequence intervals in which the entire length of the interval was present on more than one sequence in the cluster were identified, and intervals thus identified were considered to be equivalent by transitivity. For example, if an interval was present on a cDNA and two genomic sequences, then all three intervals were considered to be equivalent. This process allows unrelated but consecutive genomic sequences to be brought together, bridged by cDNA sequence. Intervals thus identified were then “stitched” together by the stitching algorithm in the order that they appear along their parent sequences to generate the longest possible sequence, as well as sequence variants. Linkages between intervals which proceed along one type of parent sequence (cDNA to cDNA or genomic sequence to genomic sequence) were given preference over linkages which change parent type (cDNA to genomic sequence). The resultant stitched sequences were translated and compared by BLAST analysis to the genpept and gbpri public databases. Incorrect ekons predicted by Genscan were corrected by comparison to the top BLAST hit from genpept. Sequences were further extended with additional cDNA sequences, or by inspection of genomic DNA, when necessary.

“Stretched” Sequences

Partial DNA sequences were extended to full length with an algorithm based on BLAST analysis. First, partial cDNAs assembled as described in Example III were queried against public databases such as the GenBank primate, rodent, mammalian, vertebrate, and eukaryote databases using the BLAST program. The nearest GenBank protein homolog was then compared by BLAST analysis to either Incyte cDNA sequences or GenScan exon predicted sequences described in Example IV. A chimeric protein was generated by using the resultant high-scoring segment pairs (HSPs) to map the translated sequences onto the GenBank protein homolog. Insertions or deletions may occur in the chimeric protein with respect to the original GenBank protein homolog. The GenBank protein homolog, the chimeric protein, or both were used as probes to search for homologous genomic sequences from the public human genome databases. Partial DNA sequences were therefore “stretched” or extended by the addition of homologous genomic sequences. The resultant stretched sequences were examined to determine whether it contained a complete gene.

VI. Chromosomal Mapping of NAAP Encoding Polynucleotides

The sequences which were used to assemble SEQ ]ID NO:59-116 were compared with sequences from the Incyte LIFESEQ database and public domain databases using BLAST and other implementations of the Smith-Waterman algorithm. Sequences from these databases that matched SEQ ID NO:59-116 were assembled into clusters of contiguous and overlapping sequences using assembly algorithms such as Phrap (Table 7). Radiation hybrid and genetic mapping data available from public resources such as the Stanford Human Genome Center (SHGC), Whitehead Institute for Genome Research (WIGR), and Genethon were used to determine if any of the clustered sequences had been previously mapped. Inclusion of a mapped sequence in a cluster resulted in the assignment of all sequences of that cluster, including its particular SEQ ID NO:, to that map location.

Map locations are represented by ranges, or intervals, of human chromosomes. The map position of an interval, in centiMorgans, is measured relative to the terminus of the chromosome's p-arm. (The centimorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Genethon which provide boundaries for radiation hybrid markers whose sequences were included in each of the clusters. Human genome maps and other resources available to the public, such as the NCBI “GeneMap'99” World Wide Web site (http://www.ncbi.nlm.nih.gov/genemap/), can be employed to determine if previously identified disease genes map within or in proximity to the intervals indicated above.

VII. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound (Sambrook and Russell, supra, ch. 7; Ausubel et al., supra, ch. 4).

Analogous computer techniques applying BLAST were used to search for identical or related molecules in databases such as GenBank or LEESEQ (Incyte Genomics). This analysis is much faster than multiple membrane-based hybridizations. In addition, the sensitivity of the computer search can be modified to determine whether any particular match is categorized as exact or similar. The basis of the search is the product score, which is defined as:

$$\frac{BLAST Score \times Percent Identity}{5 \times minimum \{ length ( Seq . 1 ) , length ( Seq . 2 ) \}}$$

The product score takes into account both the degree of similarity between two sequences and the length of the sequence match. The product score is a normalized value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP), and −4 for every mismatch. Two sequences may share more than one HSP (separated by gaps). If there is more than one HSP, then the pair with the highest BLAST score is used to calculate the product score. The product score represents a balance between fractional overlap and quality in a BLAST alignment. For example, a product score of 100 is produced only for 100% identity over the entire length of the shorter of the two sequences being compared. A product score of 70 is produced either by 100% identity and 70% overlap at one end, or by 88% identity and 100% overlap at the other. A product score of 50 is produced either by 100% identity and 50% overlap at one end, or 79% identity and 100% overlap.

Alternatively, polynucleotides encoding NAAP are analyzed with respect to the tissue sources from which they were derived. For example, some full length sequences are assembled, at least in part, with overlapping Incyte cDNA sequences (see Example III). Each cDNA sequence is derived from a cDNA library constructed from a human tissue. Each human tissue is classified into one of the following organ/tissue categories: cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract. The number of libraries in each category is counted and divided by the total number of libraries across all categories. Similarly, each human tissue is classified into one of the following disease/condition categories: cancer, cell line, developmental, inflammation, neurological, trauma, cardiovascular, pooled, and other, and the number of libraries in each category is counted and divided by the total number of libraries across all categories. The resulting percentages reflect the tissue- and disease-specific expression of cDNA encoding NAAP. cDNA sequences and cDNA library/tissue information are found in the LIFESEQ GOLD database (Incyte Genomics, Palo Alto Calif.).

VIII. Extension of NAAP Encoding Polynucleotides

Full length polynucleotides are produced by extension of an appropriate fragment of the full length molecule using oligonucleotide primers designed from this fragment. One primer was synthesized to initiate 5′ extension of the known fragment, and the other primer was synthesized to initiate 3′ extension of the known fragment. The initial primers were designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed.

High fidelity amplification was obtained by PCR using methods well known in the art. PCR was performed in 96-well plates using the PTC-200 thermal cycler (MJ Research, Inc.). The reaction mix contained DNA template, 200 nmol of each primer, reaction buffer containing Mg²⁺, (NH₄)₂SO₄, and 2-mercaptoethanol, Taq DNA polymerase (Amersham Biosciences), ELONGASE enzyme (Invitrogen), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 40C.

The concentration of DNA in each well was determined by dispensing 100 μl PICOGREEN quantitation reagent (0.25% (v/v) PICOGREEN; Molecular Probes, Eugene Oreg.) dissolved in 1× TE and 0.5 μl of undiluted PCR product into each well of an opaque fluorimeter plate (Corning Costar, Acton Mass.), allowing the DNA to bind to the reagent. The plate was scanned in a Fluoroskan II (Labsystems Oy, Helsinki, Finland) to measure the fluorescence of the sample and to quantify the concentration of DNA. A 5 μl to 10 μl aliquot of the reaction mixture was analyzed by electrophoresis on a 1% agarose gel to determine which reactions were successful in extending the sequence.

The extended nucleotides were desalted and concentrated, transferred to 384-well plates, digested with CviJI cholera virus endonuclease (Molecular Biology Research, Madison Wis.), and sonicated or sheared prior to religation into pUC 18 vector (Amershamn Biosciences). For shotgun sequencing, the digested nucleotides were separated on low concentration (0.6 to 0.8%) agarose gels, fragments were excised, and agar digested with Agar ACE (Promega). Extended clones were religated using T4 ligase (New England Biolabs, Beverly Mass.) into pUC 18 vector (Amersham Biosciences), treated with Pfu DNA polymerase (Stratagene) to fill-in restriction site overhangs, and transfected into competent E. coli cells. Transformed cells were selected on antibiotic-containing media, and individual colonies were picked and cultured overnight at 37° C. in 384-well plates in LB/2× carb liquid media.

The cells were lysed, and DNA was amplified by PCR using Taq DNA polymerase (Amersham Biosciences) and Pfu DNA polymerase (Stratagene) with the following parameters: Step 1: 94° C., 3 min; Step 2: 94° C., 15 sec; Step 3: 60 OC, 1 min; Step 4: 72° C., 2 min; Step 5: steps 2, 3, and 4 repeated 29 times; Step 6: 72° C., 5 min; Step 7: storage at 4° C. DNA was quantified by PICOGREEN reagent (Molecular Probes) as described above. Samples with low DNA recoveries were reamplified using the same conditions as described above. Samples were diluted with 20% dimethysulfoxide (1:2, v/v), and sequenced using DYENAMIC energy transfer sequencing primers and the DYENAMIC DIRECT kit (Amersham Biosciences) or the ABI PRISM BIGDYE Terminator cycle sequencing ready reaction kit (Applied Biosystems).

In like manner, full length polynucleotides are verified using the above procedure or are used to obtain 5′ regulatory sequences using the above procedure along with oligonucleotides designed for such extension, and an appropriate genomic library.

IX. Identification of Single Nucleotide Polymorphisms in NAAP Encoding Polynucleotides

Common DNA sequence variants known as single nucleotide polymorphisms (SNPs) were identified in SEQ ID NO:59-116 using the LIFESEQ database (Incyte Genomics). Sequences from the same gene were clustered together and assembled as described in Example III, allowing the identification of all sequence variants in the gene. An algorithm consisting of a series of filters was used to distinguish SNPs from other sequence variants. Preliminary filters removed the majority of basecall errors by requiring a minimum Phred quality score of 15, and removed sequence alignment errors and errors resulting from improper trimming of vector sequences, chimeras, and splice variants. An automated procedure of advanced chromosome analysis analysed the original chromatogram files in the vicinity of the putative SNP. Clone error filters used statistically generated algorithms to identify errors introduced during laboratory processing, such as those caused by reverse transcriptase, polymerase, or somatic mutation. Clustering error filters used statistically generated algorithms to identify errors resulting from clustering of close homologs or pseudogenes, or due to contamination by non-human sequences. A final set of filters removed duplicates and SNPs found in immunoglobulins or T-cell receptors.

Certain SNPs were selected for further characterization by mass spectrometry using the high throughput MASSARRAY system (Sequenom, Inc.) to analyze allele frequencies at the SNP sites in four different human populations. The Caucasian population comprised 92 individuals (46 male, 46 female), including 83 from Utah, four French, three Venezualan, and two Amish individuals. The African population comprised 194 individuals (97 male, 97 female), all African Americans. The Hispanic population comprised 324 individuals (162 male, 162 female), all Mexican Hispanic. The Asian population comprised 126 individuals (64 male, 62 female) with a reported parental breakdown of 43% Chinese, 31% Japanese, 13% Korean, 5% Vietnamese, and 8% otlier Asian. Allele frequencies were first analyzed in the Caucasian population; in some cases those SNPs which showed no allelic variance in this population were not further tested in the other three populations.

X. Labeling and Use of Individual Hybridization Probes

Hybridization probes derived from SEQ ID NO:59-116 are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 software (National Biosciences) and labeled by combining 50 pmol of each oligomer, 250 μCi of [γ-³²P] adenosine triphosphate (Amersham Biosciences), and T4 polynucleotide kinase DuPont NEN, Boston Mass.). The labeled oligonucleotides are substantially purified using a SEPHADEX G-25 superfine size exclusion dextran bead column (Amersham Biosciences). An aliquot containing 10⁷ counts per minute of the labeled probe is used in a typical membrane-based hybridization analysis of human genomic DNA digested with one of the following endonucleases: Ase I, Bgl II, Eco RI, Pst I, Xba I, or Pvu II (DuPont NEN).

The DNA from each digest is fractionated on a 0.7% agarose gel and transferred to nylon membranes (Nytran Plus, Schleicher & Schuell, Durham N. H.). Hybridization is carried out for 16 hours at 40° C. To remove nonspecific signals, blots are sequentially washed at room temperature under conditions of up to, for example, 0.1× saline sodium citrate and 0.5% sodium dodecyl sulfate. Hybridization patterns are visualized using autoradiography or an alternative imaging means and compared.

XI. Microarrays

The linkage or synthesis of array elements upon a microarray can be achieved utilizing photolithography, piezoelectric printing (ink-jet printing; see, e.g., Baldeschweiler et al., supra), mechanical microspotting technologies, and derivatives thereof. The substrate in each of the aforementioned technologies should be uniform and solid with a non-porous surface (Schena, M., ed. (1999) DNA Microarrays: A Practical Approach, Oxford University Press, London). Suggested substrates include silicon, silica, glass slides, glass chips, and silicon wafers. Alternatively, a procedure analogous to a dot or slot blot may also be used to arrange and link elements to the surface of a substrate using thermal, UV, chemical, or mechanical bonding procedures. A typical array may be produced using available methods and machines well known to those of ordinary skill in the art and may contain any appropriate number of elements (Schena, M. et al. (1995) Science 270:467-470; Shalon, D. et al. (1996) Genome Res. 6:639-645; Marshall, A. and J. Hodgson (1998) Nat. Biotechnol. 16:27-31).

Full length cDNAs, Expressed Sequence Tags (ESTs), or fragments or oligomers thereof may comprise the elements of the microarray. Fragments or oligomers suitable for hybridization can be selected using software well known in the art such as LASERGENE software (DNASTAR). The array elements are hybridized with polynucleotides in a biological sample. The polynucleotides in the biological sample are conjugated to a fluorescent label or other molecular tag for ease of detection. After hybridization, nonhybridized nucleotides from the biological sample are removed, and a fluorescence scanner is used to detect hybridization at each array element. Alternatively, laser desorbtion and mass spectrometry may be used for detection of hybridization. The degree of complementarity and the relative abundance of each polynucleotide which hybridizes to an element on the microarray may be assessed. In one embodiment, microarray preparation and usage is described in detail below.

Tissue or Cell Sample Preparation

Total RNA is isolated from tissue samples using the guanidinium thiocyanate method and poly(A)⁺ RNA is purified using the oligo-(dT) cellulose method. Each poly(A)⁺ RNA sample is reverse transcribed using MMLV reverse-transcriptase, 0.05 pg/μl oligo-(dT) primer (21mer), 1× first strand buffer, 0.03 units/μl RNase inhibitor, 500 μM dATP, 500 μM dGTP, 500 μM dTTP, 40 μM dCTP, 40 μM dCTP-Cy3 (BDS) or dCTP-Cy5 (Amersham Biosciences). The reverse transcription reaction is performed in a 25 ml volume containing 200 ng poly(A)⁺ RNA with GEMBRIGHT kits (Incyte Genomics). Specific control poly(A)⁺ RNAs are synthesized by in vitro transcription from non-coding yeast genomic DNA. After incubation at 37° C. for 2 hr, each reaction sample (one with Cy3 and another with Cy5 labeling) is treated with 2.5 ml of 0.5M sodium hydroxide and incubated for 20 minutes at 85° C. to the stop the reaction and degrade the RNA. Samples are purified using two successive CHROMA SPIN 30 gel filtration spin columns (Clontech, Palo Alto Calif.) and after combining, both reaction samples are ethanol precipitated using 1 ml of glycogen (1 mg/ml), 60 ml sodium acetate, and 300 ml of 100% ethanol. The sample is then dried to completion using a SpeedVAC (Savant Instruments Inc., Holbrook N.Y.) and resuspended in 14 μl 5×SSC/0.2% SDS.

Microarray Preparation

Sequences of the present invention are used to generate array elements. Each array element is amplified from bacterial cells containing vectors with cloned cDNA inserts. PCR amplification uses primers complementary to the vector sequences flanking the cDNA insert. Array elements are amplified in thirty cycles of PCR from an initial quantity of 1-2 ng to a final quantity greater than 5 μg. Amplified array elements are then purified using SEPHACRYL-400 (Amersham Biosciences).

Purified array elements are immobilized on polymer-coated glass slides. Glass microscope slides (Corning) are cleaned by ultrasound in 0.1% SDS and acetone, with extensive distilled water washes between and after treatments. Glass slides are etched in 4% hydrofluoric acid (VWR Scientific Products Corporation (VWR), West Chester Pa.), washed extensively in distilled water, and coated with 0.05% aminopropyl silane (Sigma) in 95% ethanol. Coated slides are cured in a 110° C. oven.

Array elements are applied to the coated glass substrate using a procedure described in U.S. Pat. No. 5,807,522, incorporated herein by reference. 1 μl of the array element DNA, at an average concentration of 100 ng/μl, is loaded into the open capillary printing element by a high-speed robotic apparatus. The apparatus then deposits about 5 nl of array element sample per slide.

Micro arrays are UV-crosslinked using a STRATALINKER UV-crosslinker (Stratagene). Microarrays are washed at room temperature once in 0.2% SDS and three times in distilled water. Non-specific binding sites are blocked by incubation of microarrays in 0.2% casein in phosphate buffered saline (PBS) (Tropix, Inc., Bedford Mass.) for 30 minutes at 60° C. followed by washes in 0.2% SDS and distilled water as before.

Hybridization

Hybridization reactions contain 9 μl of sample mixture consisting of 0.2 μg each of Cy3 and Cy5 labeled cDNA synthesis products in 5×SSC, 0.2% SDS hybridization buffer. The sample mixture is heated to 65° C. for 5 minutes and is aliquoted onto the microarray surface and covered with an 1.8 cm² coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hours at 60° C. The arrays are washed for 10 min at 45° C. in a first wash buffer (1×SSC, 0.1% SDS), three times for 10 minutes each at 45° C. in a second wash buffer (0.1×SSC), and dried.

Detection

Reporter-labeled hybridization complexes are detected with a microscope equipped with an Innova 70 mixed gas 10 W laser (Coherent, Inc., Santa Clara Calif.) capable of generating spectral lines at 488 nm for excitation of Cy3 and at 632 nm for excitation of Cy5. The excitation laser light is focused on the array using a 20× microscope objective (Nikon, Inc., Melville N.Y.). The slide containing the array is placed on a computer-controlled X-Y stage on the microscope and raster-scanned past the objective. The 1.8 cm×1.8 cm array used in the present example is scanned with a resolution of 20 micrometers.

In two separate scans, a mixed gas multiline laser excites the two fluorophores sequentially. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the sample mixture at a known concentration. A specific location on the array contains a complementary DNA sequence, allowing the intensity of the signal at that location to be correlated with a weight ratio of hybridizing species of 1:100,000. When two samples from different sources (e.g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit RTI-835H analog-to-digital (A/D) conversion board (Analog Devices, Inc., Norwood Mass.) installed in an IBM-compatible PC computer. The digitized data are displayed as an image where the signal intensity is mapped using a linear 20-color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each fluorophore's emission spectrum.

A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS gene expression analysis program (Incyte Genomics). Array elements that exhibit at least about a two-fold change in expression, a signal-to-background ratio of at least about 2.5, and an element spot size of at least about 40%, are considered to be differentially expressed.

Expression

SEQ ID NO:65 showed differential expression in cancer cell lines versus non-cancerous cell lines, as determined by microarray analysis. For example, the expression of SEQ ID NO:65 was decreased by at least two fold in breast tumor cell lines each isolated from pleural effusion from donors at different stages of tumor progression and malignant transformation when grown in one of two different chemically defined, serum-free media both supplemented with growth factors and growth hormones. Therefore, SEQ ID NO:65 is useful in diagnostic assays for breast cancer.

Normal breast cell lines are obtained as follows. Primary mammary gland cells are isolated from a donor with fibrocystic breast disease. Humorous breast cell lines are obtained as follows. Breast carcinoma cells are derived in vitro from cells emigrating from a tumor. Alternately, breast tumor cells are isolated from invasive tumor of donors. Further, nonmalignant or malignant primary breast adenocarcinoma cells are obtained from the pleural effusion of donors.

Further, the expression of SEQ ID NO:65 was decreased at least two-fold in treated human adipocytes from obese and normal donors when compared to non-treated adipocytes from the same donors. The normal human primary subcutaneous preadipocytes were isolated from adipose tissue of a 28-year-old healthy female with a body mass index (BMI) of 23.59. The obese human primary subcutaneous preadipocytes were isolated from adipose tissue of a 40-year-old healthy female with a body mass index (BMI) of 32.47. The preadipocytes were cultured and induced to differentiate into adipocytes by culturing them in the differentiation medium containing the active components, PPAR-γ agonist and human insulin. Human preadipocytes were treated with human insulin and PPAR-γ agonist for three days and subsequently were switched to medium containing insulin for 24 hours, 48 hours, four days, 8 days or 15 days before the cells were collected for analysis. Differentiated adipocytes were compared to untreated preadipocytes maintained in culture in the absence of inducing agents. Between 80% and 90% of the preadipocytes finally differentiated to.adipocytes as observed under phase contrast microscope. Thus, SEQ ID NO:65 is useful for the diagnosis, prognosis, or treatment of diabetes mellitus and other disorders, such as obesity, hypertension, atherosclerosis, polycystic ovarian syndrome, and cancers including breast, prostate, and colon.

For example, SEQ ID NO:72-74 showed differential expression in tumorous tissue versus non-tumorous tissues, as determined by microarray analysis. The expression of cDNAs from lung tumor tissue from several donors was compared with that of normal lung tissue from the same donor, respectively. Array elements that exhibited about at least a two-fold change in expression and a signal intensity over 250 units, a signal-to-background ratio of a least 2.5, and an element spot size of at least 40% were identified as differentially expressed using the GEMTOOLS program (Incyte Genomics).

The expression of SEQ ID NO:72 was increased at least two-fold in lung squamous cell carcinoma when matched with normal tissue from the same donor. The tumorous lung tissue was obtained from the lung of a 66-year-old male with lung squamous cell carcinoma. Normal tissue was obtained from grossly uninvolved lung tissue from the same donor. Therefore, SEQ ID NO:72 is useful in diagnostic assays for lung squamous cell carcinoma.

Alternately, the expression of SEQ ID NO:73 was decreased at least 2.7-fold in lung adenocarcinoma when matched with normal tissue from the same donor. The tumorous lung tissue was obtained from the right lung of a 60-year old donor with moderately differentiated adenocarcinoma. Normal tissue was obtained from grossly uninvolved tissue from the right lung from the same donor. Therefore, SEQ ID NO:73 is useful in diagnostic assays for lung adenocarcinoma. Further, the expression of SEQ ID NO:74 was increased at least 2.7-fold in lung adenocarcinoma when matched with normal tissue from the same donor. The tumorous lung tissue was obtained from the lung of a 66-year old female with lung adenocarcinoma. Normal tissue was obtained from grossly uninvolved tissue from grossly uninvolved lung tissue from the same donor. The expression of SEQ ID NO:74 was increased at least 3.2-fold in lung squamous cell carcinoma from two donors when matched with normal tissue from the same donor. In one case, the tumorous lung tissue was obtained from the lung of a 66-year-old male with lung squamous cell carcinoma. In the other case, the tumorous lung tissue was obtained from the lung of a 73-year old male with lung squamous cell carcinoma. Normal tissue was obtained from grossly uninvolved lung tissue from the same donor, respectively. Therefore, SEQ ID NO:74 is useful in diagnostic assays for lung adenocarcinoma and squamous cell carcinoma.

For example, SEQ ID NO:79 showed increased expression in colon tissue affected by colon cancer versus normal colon tissue as determined by microarray analysis. Gene expression profiles were obtained by comparing normal colon tissue from a 67 year-old donor with moderately differentiated adenocarcinoma (Dukes B, TNM classification) to cancer-affected colon tissue from the same donor. Samples were provided by the Huntsman Cancer Institute. Therefore, SEQ ID NO:79 is useful in diagnostic assays for disorders of cell proliferation including colon cancer.

For example, SEQ ID NO:79 showed decreased expression in ovary tissue affected by ovarian cancer versus normal ovary tissue as determined by microarray analysis. A normal ovary from a 79 year-old female donor was compared to an ovarian tumor from the same donor. Samples were provided by the Huntsman Cancer Institute. Therefore, SEQ ID NO:79 is useful in diagnostic assays for disorders of cell proliferation including ovarian cancer.

For example, SEQ ID NO:79 showed decreased expression in C3A cells treated with dexamethasone, versus untreated C3A cells, as determined by microarray analysis. The human C3A cell line is a clonal derivative of HepG2/C3 (hepatoma cell line, isolated from a 15-year-old male with liver tumor), which was selected for strong contact inhibition of growth. Early Confluent C3A cells were treated with dexamethasone at 1, 10, and 100 μM for 1, 3, and 6 hours. The treated cells were compared to untreated early confluent C3A cells. Therefore, SEQ ID NO:79 is useful in diagnostic assays for, and monitoring treatment of, autoimmune/inflammatory disorders.

For example, SEQ ID NO:81 showed differential expression in fibroblasts affected by Tangiers Disease (TD) versus normal fibroblasts, when both were treated with LDL cholesterol, as determined by microarray analysis. Normal and TD-derived fibroblasts were compared cultured in the presence of cholesterol and compared with the same cell type cultured in the absence of cholesterol. Human fibroblasts were obtained from skin explants from both normal subjects and two patients with homozygous ID. Cell lines were immortalized by transfection with human papillomavirus 16 genes E6 and E7 and a neomycin resistance selectable marker, and TD was confirmed in TD-derived cells by reduced apoA-I mediated tritiated cholesterol efflux. Therefore, SEQ ID NO:81 is useful in diagnostic assays for autoimmune/inflammatory disorders including Tangier Disease.

For example, SEQ ID NO:93 showed differential expression in mammary cells affected by breast carcinoma versus nonmalignant mammary epithelial cells as determined by microarray analysis. The gene expression profile of a nonmalignant mammary epithelial cell line was compared to the gene expression profiles of breast carcinoma lines at different stages of tumor progression. Cell lines compared included: a) MCF-10A, a breast mammary gland cell line isolated from a 36-year-old woman with fibrocystic breast disease; b) MCF7, a nonmalignant breast adenocarcinoma cell line isolated from the pleural effusion of a 69-year-old female; c) T-47D, a breast carcinoma cell line isolated from a pleural effusion obtained from a 54-year-old female with an infiltrating ductal carcinoma of the breast; d) Sk-BR-3, a breast adenocarcinoma cell line isolated from a malignant pleural effusion of a 43-year-old female; e) BT-20, a breast carcinoma cell line derived in vitro from tumor mass isolated from a 74-year-old female; f) MDA-mb-231, a breast tumor cell line isolated from the pleural effusion of a 51-year old female; and g) MDA-mb-435S, a spindle shaped strain that evolved from the parent line (435) isolated from the pleural effusion of a 3 1-year-old female with metastatic, ductal adenocarcinoma of the breast.

The cells were grown in the supplier's recommended medium to 70-80% confluence prior to RNA harvest. Expression was decreased by at least two-fold in 4 of the 6 breast carcinoma cell lines as compared to the nonmalignant mammary epithelial cell line. Therefore, SEQ 1D NO:93 is useful in diagnostic assays for and monitoring treatment of, cell proliferative disorders including breast carcinoma.

As another example, SEQ ID NO:94 showed decreased expression in tissue affected by adenocarcinoma versus normal tissue as determined by microarray analysis. A sample of tissue right lung tissue that showed moderately differentiated adenocarcinoma of was compared to grossly uninvolved lung tissue from the same donor (Huntsman Cancer Institute, Salt Lake City, Utah). Therefore, SEQ ID NO:94 is useful in diagnostic assays for, and monitoring treatment of, cell proliferative disorders including adenocarcinoma.

As another example, SEQ ID NO:98 showed decreased expression in stimulated dendritic cells treated with CD40 antibodies versus stimulated dendritic cells not treated with CD40 antibodies, as determined by microarray analysis. Human monocytic dendritic cells (mDCs) were derived in vitro from the adherent cellular fraction of the peripheral blood of 4 healthy volunteer donors. The adherent leukocytes, mostly monocytes, were incubated for 13 days in the presence of recombinant interleukin-4 (10 ng/ml) and granulocyte/macrophage colony stimulating factor (10 ng/ml). The differentiated mDCs were collected after 13 days from the non-adherent cellular fraction and activated in the presence of soluble mouse anti-human CD40 antibodies for 2, 8, and 24 hours. The anti-CD40 treated mDCs were compared to untreated mDCs. Therefore, SEQ ED NO:98 is useful in diagnostic assays for, and monitoring treatment of, autoimmune/inflammatory disorders.

As another example, SEQ ID NO:100 showed decreased expression in cells treated with tumor necrosis factor alpha TNF-α), which mediates the inflammatory response through activation of signal transduction pathways, versus untreated cells as determined by microarray analysis. Human aortic endothelial cells (HMVECdNeos) were grown to 85% confluence and then treated for 1, 2, 4, 8, and 24 hours with tumor necrosis factor alpha (TNF-α). TNF-α -treated cells were compared to untreated HMVECdNeos collected at 85% confluence (0 hour). Therefore, SEQ ID NO:100 is useful in diagnostic assays for, and monitoring treatment of, cell proliferative disorders.

In order to evaluate RNA expression, HMVECdNeo cells were grown to 85% confluency and then treated with TNF-α (10 ng/ml) for 2, 4, 8, and 24 hours. TNF-α-treated cells were compared to untreated HMVECdNeos collected at 85% confluency (0 hour). The expression of SEQ ID NO:108 was underexpressed by at least two-fold in TNF-α-treated versus untreated cells at the last three time points tested. Therefore, SEQ ID NO:108 maybe useful in disease staging and diagnostic assays for cell proliferative and inflammatory disorders, including those involving nucleic acid-associated proteins.

Region-specific RNA expression in human brain tissue was evaluated using specific dissected brain regions from a non-demented human female brain. Brain regions were then pooled and used as the control. Specific brain regions were then compared to the mixed brain control. The mixed brain control was reconstituted from the purified mRNA isolated from the major regions of the brain. The expression of SEQ ID NO:109 was underexpressed by at least two-fold in the dentate nuclear brain tissue as compared to the mixed brain control tissue. Therefore, SEQ ID NO:109 maybe useful in disease staging and diagnostic assays for cell proliferative and/or neurological disorders, including those involving nucleic acid-associated proteins.

XII. Complementary Polynucleotides

Sequences complementary to the NAAP-encoding sequences, or any parts thereof, are used to detect, decrease, or inhibit expression of naturally occurring NAAP. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of NAAP. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the NAAP-encoding transcript.

XIII. Expression of NAAP

Expression and purification of NAAP is achieved using bacterial or virus-based expression systems. For expression of NAAP in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express NAAP upon induction with isopropyl beta-D-thiogalactopyranoside (WIUG). Expression of NAAP in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding NAAP by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera frugiperda (Sf9) insect cells in most cases, or human hepatocytes, in some cases. Infection of the latter requires additional genetic modifications to baculovirus (Engelhard, E. K. et al. (1994) Proc. Natl. Acad. Sci. USA 91:3224-3227; Sandig, V. et al. (1996) Hum. Gene Ther. 7:1937-1945).

In most expression systems, NAAP is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Amersham Biosciences). Following purification, the GST moiety can be proteolytically cleaved from NAAP at specifically engineered sites. FLAG, an 8-amino acid peptide, enables immunoaffinity purification using conmmercially available monoclonal and polyclonal anti-FLAG antibodies (Eastman Kodak). 6-His, a stretch of six consecutive histidine residues, enables purification on metal-chelate resins (QIAGEN). Methods for protein expression and purification are discussed in Ausubel et al. (supra, ch. 10 and 16). Purified NAAP obtained by these methods can be used directly in the assays shown in Examples XVII, XVIII, XIX, and XX, where applicable.

XIV. Functional Assays

NAAP function is assessed by expressing the sequences encoding NAAP at physiologically elevated levels in mammalian cell culture systems. cDNA is subcloned into a mammalian expression vector containing a strong promoter that drives high levels of cDNA expression. Vectors of choice include PCMV SPORT plasmid (Invitrogen, Carlsbad Calif.) and PCR3.1 plasmid (Invitrogen), both of which contain the cytomegalovirus promoter. 5-10 μg of recombinant vector are transiently transfected into a human cell line, for example, an endothelial or hematopoietic cell line, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; Clontech), CD64, or a CD64-GFP fusion protein. Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994; Flow Cytometry, Oxford, New York N.Y.).

The influence of NAAP on gene expression can be assessed using highly purified populations of cells transfected with sequences encoding NAAP and either CD64 or CD64-GFP. CD64 and CD64-GFP are expressed on the surface of transfected cells and bind to conserved regions of human immunoglobulin G (IgG). Transfected cells are efficiently separated from nontransfected cells using 30 magnetic beads coated with either human IgG or antibody against CD64 (DYNAL, Lake Success N.Y.). mRNA can be purified from the cells using methods well known by those of skill in the art.

Expression of mRNA encoding NAAP and other genes of interest can be analyzed by northern analysis or microarray techniques.

XV. Production of NAAP Specific Antibodies

NAAP substantially purified using polyacrylamide gel electrophoresis (PAGE; see, e.g., Harrington, M. G. (1990) Methods Enzymol. 182:488-495), or other purification techniques, is used to immunize animals (e.g., rabbits, mice, etc.) and to produce antibodies using standard protocols.

Alternatively, the NAAP amino acid sequence is analyzed using LASERGENE software (DNASTAR) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art (Ausubel et al., supra, ch. 11).

Typically, oligopeptides of about 15 residues in length are synthesized using an ABI 431A peptide synthesizer (Applied Biosystems) using FMOC chemistry and coupled to KLH (Sigma-Aldrich, St. Louis Mo.) by reaction with N-maleimidobenzoyl-N-hydroxysuccinimide ester (MB S) to increase immunogenicity (Ausubel et al., supra). Rabbits are immunized with the oligopeptide-KLH complex in complete Freund's adjuvant. Resulting antisera are tested for antipeptide and anti-NAAP activity by, for example, binding the peptide or NAAP to a substrate, blocking with 1% BSA, reacting with rabbit antisera, washing, and reacting with radio-iodinated goat anti-rabbit IgG.

XVI. Purification of Naturally Occurring NAAP Using Specific Antibodies

Naturally occurring or recombinant NAAP is substantially purified by immunoaffinity chromatography using antibodies specific for NAAP. An immunoaffinity column is constructed by covalently coupling anti-NAAP antibody to an activated chromatographic resin, such as CNBr-activated SEPHAROSE (Amersham Biosciences). After the coupling, the resin is blocked and washed according to the manufacturer's instructions.

Media containing NAAP are passed over the immunoaffinity column, and the column is washed under conditions that allow the preferential absorbance of NAAP (e.g., high ionic strength buffers in the presence of detergent). The column is eluted under conditions that disrupt antibody/NAAP binding (e.g., a buffer of pH 2 to pH 3, or a high concentration of a chaotrope, such as urea or thiocyanate ion), and NAAP is collected.

XVII. Identification of Molecules Which Interact with NAAP

NAAP, or biologically active fragments thereof, are labeled with ¹²⁵I Bolton-Hunter reagent (Bolton, A. E. and W. M. Hunter (1973) Biochem. J. 133:529-539). Candidate molecules previously arrayed in the wells of a multi-well plate are incubated with the labeled NAAP, washed, and any wells with labeled NAAP complex are assayed. Data obtained using different concentrations of NAAP are used to calculate values for the number, affinity, and association of NAAP with the candidate molecules.

Alternatively, molecules interacting with NAAP are analyzed using the yeast two-hybrid system as described in Fields, S. and O. Song (1989; Nature 340:245-246), or using commercially available kits based on the two-hybrid system, such as the MATCHMAKER system (Clontech).

NAAP may also be used in the PATHCALLING process (CuraGen Corp., New Haven Conn.) which employs the yeast two-hybrid system in a high-throughput manner to determine all interactions between the proteins encoded by two large libraries of genes (Nandabalan, K. et al. (2000) U.S. Pat. No. 6,057,101).

XVIII. Demonstration of NAAP Activity

NAAP activity is measured by its ability to stimulate transcription of a reporter gene (Liu, H. Y. et al. (1997) EMBO J. 16:5289-5298). The assay entails the use of a well characterized reporter gene construct, LexAₒₚ-LacZ, that consists of LexA DNA transcriptional control elements (LexAₒₚ) fused to sequences encoding the E. coli LacZ enzyme. The methods for constructing and expressing fusion genes, introducing them into cells, and measuring LacZ enzyme activity, are well known to those skilled in the art. Sequences encoding NAAP are cloned into a plasmid that directs the synthesis of a fusion protein, LexA-NAAP, consisting of NAAP and a DNA binding domain derived from the LexA transcription factor. The resulting plasmid, encoding a LexA-NAAP fusion protein, is introduced into yeast cells along with a plasmid containing the LexAₒₚ-LacZ reporter gene. The amount of LacZ enzyme activity associated with LexA-NAAP transfected cells, relative to control cells, is proportional to the amount of transcription stimulated by the NAAP.

Alternatively, NAAP activity is measured by its ability to bind zinc. A 5-10 μM sample solution in 2.5 mM ammonium acetate solution at pH 7.4 is combined with 0.05 M zinc sulfate solution (Aldrich, Milwaukee Wis.) in the presence of 100 μM dithiothreitol with 10% methanol added. The sample and zinc sulfate solutions are allowed to incubate for 20 minutes. The reaction solution is passed through a VYDAC column (Grace Vydac, Hesperia, Calif.) with approximately 300 Angstrom bore size and 5 μM particle size to isolate zinc-sample complex from the solution, and into a mass spectrometer (PE Sciex, Ontario, Canada). Zinc bound to sample is quantified using the functional atomic mass of 63.5 Da observed by Whittal, R. M. et al. ((2000) Biochemistry 39:8406-8417).

In the alternative, a method to determine nucleic acid binding activity of NAAP involves a polyacrylamide gel mobility-shift assay. In preparation for this assay, NAAP is expressed by transforming a mammalian cell line such as COS7, HeLa or CHO with a eukaryotic expression vector containing NAAP cDNA. The cells are incubated for 48-72 hours after transformation under conditions appropriate for the cell line to allow expression and accumulation of NAAP. Extracts containing solubilized proteins can be prepared from cells expressing NAAP by methods well known in the art. Portions of the extract containing NAAP are added to [³²P]-labeled RNA or DNA. Radioactive nucleic acid can be synthesized in vitro by techniques well known in the art. The mixtures are incubated at 25° C. in the presence of RNase- and DNase-inhibitors under buffered conditions for 5-10 minutes. After incubation, the samples are analyzed by polyacrylamide gel electrophoresis followed by autoradiography. The presence of a band on the autoradiogram indicates the formation of a complex between NAAP and the radioactive transcript. A band of similar mobility will not be present in samples prepared using control extracts prepared from untransformed cells.

In the alternative, a method to determine methylase activity of NAAP measures transfer of radiolabeled methyl groups between a donor substrate and an acceptor substrate. Reaction mixtures (50 μl final volume) contain 15 mM HEPES, pH 7.9, 1.5 mM MgCl₂, 10 mM dithiothreitol, 3% polyvinylalcohol, 1.5 μCi [methyl-³H]AdoMet (0.375 μM AdoMet) (DuPont-NEN), 0.6 μg NAAP, and acceptor substrate (e.g., 0.4 μg [³⁵S]RNA, or 6-mercaptopurine (6-MP) to 1 mM final concentration). Reaction mixtures are incubated at 30° C. for 30 minutes, then 65° C. for 5 minutes.

Analysis of [methyl-³H]RNA is as follows: (1) 50 μl of 2× loading buffer (20 mM Tris-HCl, pH 7.6, 1 M LiCl, 1 mM EDTA, 1% sodium dodecyl sulphate (SDS)) and 50 μl oligo d(T)-cellulose (10 mg/ml in 1× loading buffer) are added to the reaction mixture, and incubated at ambient temperature with shaking for 30 minutes. (2) Reaction mixtures are transferred to a 96-well filtration plate attached to a vacuum apparatus. (3) Each sample is washed sequentially with three 2.4 ml aliquots of 1× oligo d(T) loading buffer containing 0.5% SDS, 0.1% SDS, or no SDS. (4) RNA is eluted with 300 μl of water into a 96-well collection plate, transferred to scintillation vials containing liquid scintillant, and radioactivity determined.

Analysis of [methyl-³H]6-MP is as follows: (1) 500 μl 0.5 M borate buffer, pH 10.0, and then 2.5 ml of 20% (v/v) isoamyl alcohol in toluene are added to the reaction mixtures. (2) The samples are mixed by vigorous vortexing for ten seconds. (3) After centrifugation at 700g for 10 minutes, 1.5 ml of the organic phase is transferred to scintillation vials containing 0.5 ml absolute ethanol and liquid scintillant, and radioactivity determined. (4) Results are corrected for the extraction of 6-MP into the organic phase (approximately 41%).

In the alternative, type I topoisomerase activity of NAAP can be assayed based on the relaxation of a supercoiled DNA substrate. NAAP is incubated with its substrate in a buffer lacking Mg²⁺ and ATP?, the reaction is terminated, and the products are loaded on an agarose gel. Altered topoisomers can be distinguished from supercoiled substrate electrophoretically. This assay is specific for type I topoisomerase activity because Mg²⁺ and ATP are necessary cofactors for type II topoisomerases.

Type II topoisomerase activity of NAAP can be assayed based on the decatenation of a kinetoplast DNA (KDNA) substrate. NAAP is incubated with KDNA, the reaction is terminated, and the products are loaded on an agarose gel. Monomeric circular KDNA can be distinguished from catenated KDNA electrophoretically. Kits for measuring type I and type II topoisomerase activities are available commercially from Topogen (Columbus Ohio).

ATP-dependent RNA helicase unwinding activity of NAAP can be measured by the method described by Zhang and Grosse (1994; Biochemistry 33:3906-3912). The substrate for RNA unwinding consists of ³²P-labeled RNA composed of two RNA strands of 194 and 130 nucleotides in length containing a duplex region of 17 base-pairs. The RNA substrate is incubated together with ATP, Mg²⁺, and varying amounts of NAAP in a Tris-HCl buffer, pH 7.5, at 37° C. for 30 minutes. The single-stranded RNA product is then separated from the double-stranded RNA substrate by electrophoresis through a 10% SDS-polyacrylamide gel, and quantitated by autoradiography. The amount of single-stranded RNA recovered is proportional to the amount of NAAP in the preparation.

In the alternative, NAAP function is assessed by expressing the sequences encoding NAAP at physiologically elevated levels in mammalian cell culture systems. cDNA is subcloned into a mammalian expression vector containing a strong promoter that drives high levels of cDNA expression. Vectors of choice include pCMV SPORT (Life Technologies) and pCR3.1 (Invitrogen Corporation, Carlsbad Calif.), both of which contain the cytomegalovirus promoter. 5-10 μg of recombinant vector are transiently transfected into a human cell line, preferably of endothelial or hematopoietic origin, using either liposome formulations or electroporation. 1-2 μg of an additional plasmid containing sequences encoding a marker protein are co-transfected.

Expression of a marker protein provides a means to distinguish transfected cells from nontransfected cells and is a reliable predictor of cDNA expression from the recombinant vector. Marker proteins of choice include, e.g., Green Fluorescent Protein (GFP; CLONTECH), CD64, or a CD64-GFP fusion protein. Flow cytometry (FCM), an automated laser optics-based technique, is used to identify transfected cells expressing GFP or CD64-GFP and to evaluate the apoptotic state of the cells and other cellular properties.

FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994) Flow Cytometry, Oxford, New York N.Y.

The influence of NAAP on gene expression can be assessed using highly purified populations of cells transfected with sequences encoding NAAP and either CD64 or CD64-GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Inc., Lake Success N.Y.). mRNA can be purified from the cells using methods well known by those of skill in the art. Expression of mRNA encoding NAAP and other genes of interest can be analyzed by northern analysis or microarray techniques.

Pseudouridine synthase activity of NAAP is assayed using a tritium (³H) release assay modified from Nurse et al. ((1995) RNA 1:102-112), which measures the release of ³H from the C₅ position of the pyrimidine component of uridylate (U) when ³H-radiolabeled U in RNA is isomerized to pseudouridine (ψ). A typical 500 μl assay mixture contains 50 mM HEPES buffer (pH 7.5), 100 mM ammonium acetate, 5 mM dithiothreitol, 1 mM EDTA, 30 units RNase inhibitor, and 0.1-4.2 μM [5-³H]tRNA (approximately 1 μCi/nmol tRNA). The reaction is initiated by the addition of <5 μl of a concentrated solution of NAAP (or sample containing NAAP) and incubated for 5 min at 37° C. Portions of the reaction mixture are removed at various times (up to 30 min) following the addition of NAAP and quenched by dilution into 1 ml 0.1 M HCl containing Norit-SA3 (12% w/v). The quenched reaction mixtures are centrifuged for S min at maximum speed in a microcentrifuge, and the supernatants are filtered through a plug of glass wool. The pellet is washed twice by resuspension in 1 ml 0.1 M HCl, followed by centrifugation. The supernatants from the washes are separately passed through the glass wool plug and combined with the original filtrate. A portion of the combined filtrate is mixed with scintillation fluid (up to 10 ml) and counted using a scintillation counter. The amount of ³H released from the RNA and present in the soluble filtrate is proportional to the amount of peudouridine synthase activity in the sample (Ramamurthy, V. (1999) J. Biol. Chem. 274:22225-22230).

In the alternative, pseudouridine synthase activity of NAAP is assayed at 30° C. to 37° C. in a mixture containing 100 mM Tris-HCl (pH 8.0), 100 mM ammonium acetate, 5 mM MgCl₂, 2 mM dithiothreitol, 0.1 mM EDTA, and 1-2 fmol of [³²P]-radiolabeled runoff transcripts (generated in vitro by an appropriate RNA polymerase, i.e., T7 or SP6) as substrates. NAAP is added to initiate the reaction or omitted from the reaction in control samples. Following incubation, the RNA is extracted with phenol-chloroform, precipitated in ethanol, and hydrolyzed completely to 3-nucleotide monophosphates using RNase T₂. The hydrolysates are analyzed by two-dimensional thin layer chromatography, and the amount of 32p radiolabel present in the ψMP and UMP spots are evaluated after exposing the thin layer chromatography plates to film or a PhosphorImager screen. Taking into account the relative number of uridylate residues in the substrate RNA, the relative amount ψMP and UMP are determined and used to calculate the relative amount of ψ per tRNA molecule (expressed in mol ψ/mol of tRNA or mol ψ/mol of tRNA/minute), which corresponds to the amount of pseudouridine synthase activity in the NAAP sample (Lecointe, F. et al. (1998) J. Biol. Chem. 273:1316-1323).

N²,N²-dimethylguanosine transferase ((m²₂G)methyltransferase) activity of NAAP is measured in a 160 μl reaction mixture containing 100 mM Tris-HCl (pH 7.5), 0.1 mM EDTA, 10 mM MgCl₂, 20 mM NH₄Cl, 1 mM dithiothreitol, 6.2 μM S-adenosyl-L-[methyl-³H]methionine (30-70 Ci/mM), 8 μg m²₂G-deficient tRNA or wild type tRNA from yeast, and approximately 100 μg of purified NAAP or a sample comprising NAAP. The reactions are incubated at 30° C. for 90 min and chilled on ice. A portion of each reaction is diluted to 1 ml in water containing 100 μg BSA. 1 ml of 2 M HCl is added to each sample and the acid insoluble products are allowed to precipitate on ice for 20 min before being collected by filtration through glass fiber filters. The collected material is washed several times with HCl and quantitated using a liquid scintillation counter. The amount of ³H incorporated into the m²₂G-deficient, acid-insoluble tRNAs is proportional to the amount of N²,N²-dimethylguanosine transferase activity in the NAAP sample. Reactions comprising no substrate tRNAs, or wild-type tRNAs that have already been modified, serve as control reactions which should not yield acid-insoluble ³H-labeled products.

Polyadenylation activity of NAAP is measured using an in vitro polyadenylation reaction. The reaction mixture is assembled on ice and comprises 10 μl of 5 mM ditiothreitol, 0.025% (v/v) NONIDET P-40, 50 mM creatine phosphate, 6.5% (w/v) polyvinyl alcohol, 0.5 unit/μl RNAGUARD (Pharmacia), 0.025 μg/μl creatine kinase, 1.25 mM cordycepin 5′-triphosphate, and 3.75 mM MgCl₂, in a total volume of 25 μl. 60 fmol of CstF, 50 fmol of CPSF, 240 fmol of PAP, 4 μl of crude or partial purified CF II and various amounts of amounts CF I are then added to the reaction mix. The volume is adjusted to 23.5 μl with a buffer containing 50 mM TrisHCl, pH 7.9, 10% (v/v) glycerol, and 0.1 mM Na-EDTA. The final ammonium sulfate concentration should be below 20 mM. The reaction is initiated (on ice) by the addition of 15 fmol of ³²P-labeled pre-mRNA template, along with 2.5 μg of unlabeled tRNA, in 1.5 μl of water. Reactions are then incubated at 30° C. for 75-90 min and stopped by the addition of 75 μl (approximately two-volumes) of proteinase K mix (0.2 M Tris-HCl, pH 7.9, 300 mM NaCl, 25 mM Na-EDTA, 2% (w/v) SDS), 1 μl of 10 mg/ml proteinase K, 0.25 μl of 20 mg/ml glycogen, and 23.75 μl of water). Following incubation, the RNA is precipitated with ethanol and analyzed on a 6% (w/v) polyacrylamide, 8.3 M urea sequencing gel. The dried gel is developed by autoradiography or using a phosphoimager. Cleavage activity is determined by comparing the amount of cleavage product to the amount of pre-mRNA template. The omission of any of the polypeptide components of the reaction and substitution of NAAP is useful for identifying the specific biological function of NAAP in pre-mRNA polyadenylation (Rüegsegger, U. et al. (1996) J. Biol. Chem. 271:6107-6113; and references within).

tRNA synthetase activity is measured as the aminoacylation of a substrate tRNA in the presence of [¹⁴C]-labeled amino acid. NAAP is incubated with [¹⁴C-labeled amino acid and the appropriate cognate tRNA (for example, [¹⁴C]alanine and tRNA^{ala}) in a buffered solution. ¹⁴C-labeled product is separated from free [¹⁴C]amino acid by chromatography, and the incorporated ¹⁴C is quantified by scintillation counter. The amount of ¹⁴C-labeled product detected is proportional to the activity of NAAP in this assay.

In the alternative, NAAP activity is measured by incubating a sample containing NAAP in a solution containing 1 mM ATP, 5 mM Hepes-KOH (pH 7.0), 2.5 mM KCl, 1.5 mM magnesium chloride, and 0.5 mM DTT along with misacylated [¹⁴C]-Glu-tRNAGln (e.g., 1 μM) and a similar concentration of unlabeled L-glutamine. Following the quenching of the reaction with 3 M sodium acetate (pH 5.0), the mixture is extracted with an equal volume of water-saturated phenol, and the aqueous and organic phases are separated by centrifugation at 15,000×g at room temperature for 1 min. The aqueous phase is removed and precipitated with 3 volumes of ethanol at −70° C. for 15 min. The precipitated aminoacyl-tRNAs are recovered by centrifugation at 15,000×g at 4° C. for 15 min. The pellet is resuspended in of 25 mM KOH, deacylated at 65° C. for 10 min., neutralized with 0.1 M HCl (to final pH 6-7), and dried under vacuum. The dried pellet is resuspended in water and spotted onto a cellulose TLC plate. The plate is developed in either isopropanol/formic acid/water or ammonia/water/chloroform/methanol. The image is subjected to densitometric analysis and the relative amounts of Glu and Gln are calculated based on the Rf values and relative intensities of the spots. NAAP activity is calculated based on the amount of Gln resulting from the transformation of Glu while acylated as Glu-tRNA^{Gln} (adapted from Curnow, A. W. et al. (1997) Proc. Natl. Acad. Sci. USA 94:11819-26).

XIX. Identification of NAAP Agonists and Antagonists

Agonists or antagonists of NAAP activation or inhibition may be tested using the assays described in section XVIII. Agonists cause an increase in NAAP activity and antagonists cause a decrease in NAAP activity.

XX. NAAP Secretion Assay

A high throughput assay may be used to identify polypeptides that are secreted in eukaryotic cells. In an example of such an assay, polypeptide expression libraries are constructed by fusing 5′-biased cDNAs to the 5′-end of a leaderless β-lactamase gene. β-lactamase is a convenient genetic reporter as it provides a high signal-to-noise ratio against low endogenous background activity and retains activity upon fusion to other proteins. A dual promoter system allows the expression of β-lactamase fusion polypeptides in bacteria or eukaryotic cells, using the lac or CMV promoter, respectively.

Libraries are first transformed into bacteria, e.g., E. coli, to identify library members that encode fusion polypeptides capable of being secreted in a prokaryotic system. Mammalian signal sequences direct the translocation of β-lactamase fusion polypeptides into the periplasm of bacteria where it confers antibiotic resistance to carbenicillin. Carbenicillin-selected bacteria are isolated on solid media, individual clones are grown in liquid media, and the resulting cultures are used to isolate library member plasmid DNA.

Mammalian cells, e.g., 293 cells, are seeded into 96-well tissue culture plates at a density of about 40,000 cells/well in 100 μl phenol red-free DME supplemented with 10% fetal bovine serum (FBS) (Life Technologies, Rockville, Md.). The following day, purified plasmid DNAs isolated from carbenicillin-resistant bacteria are diluted with 15 μl OPTI-MEM I medium (Life Technologies) to a volume of 25 μl for each well of cells to be transfected. In separate plates, 1 ;l LF2000 Reagent (Life Technologies) is diluted into 25 μl/well OPTI-MEM I. The 25 μl diluted LF2000 Reagent is then combined with the 25 μl diluted DNA, mixed briefly, and incubated for 20 minutes at room temperature. The resulting DNA-LF2000 reagent complexes are then added directly to each well of 293 cells. Cells are also transfected with appropriate control plasmids expressing either wild-type β-lactamase, leaderless β-lactamase, or, for example, CD4-fused leaderless β-lactamase. 24 hrs following transfection, about 90 μl of cell culture media are assayed at 37° C. with 100 μM Nitrocefin (Calbiochem, San Diego Calif.) and 0.5 mM oleic acid (Sigma, St. Louis, Mo.) in 10 mM phosphate buffer (pH 7.0). Nitrocefin is a substrate for β-lactamase that undergoes a noticeable color change from yellow to red upon hydrolysis. β-lactamase activity is monitored over 20 min in a microtiter plate reader at 486 nm. Increased color absorption at 486 nm corresponds to secretion of a β-lactamase fusion polypeptide in the transfected cell media, resulting from the presence of a eukaryotic signal sequence in the fusion polypeptide. Polynucleotide sequence analysis of the corresponding library member plasmid DNA is then used to identify the signal sequence-encoding cDNA. (Described in U.S. patent application Ser. No. 09/803,317, filed Mar. 9, 2001.)

Various modifications and variations of the described compositions, methods, and systems of the invention will be apparent to those skilled in the art without departing from the scope and spirit of S the invention. It will be appreciated that the invention provides novel and useful proteins, and their encoding polynucleotides, which can be used in the drug discovery process, as well as methods for using these compositions for the detection, diagnosis, and treatment of diseases and conditions. Although the invention has been described in connection with certain embodiments, it should be understood that the invention as claimed should not be unduly limited to such specific embodiments. 10 Nor should the description of such embodiments be considered exhaustive or limit the invention to the precise forms disclosed. Furthermore, elements from one embodiment can be readily recombined with elements from one or more other embodiments. Such combinations can form a number of embodiments within the scope of the invention. It is intended that the scope of the invention be defined by the following claims and their equivalents.

TABLE 1

| | | | | Incyte |
|---|---|---|---|---|
| | Polypeptide | Incyte | Polynucleotide | Polynucleotide |
| Incyte Project ID | SEQ ID NO: | Polypeptide ID | SEQ ID NO: | ID |
| 7503848 | 1 | 7503848CD1 | 59 | 7503848CB1 |
| 2608080 | 2 | 2608080CD1 | 60 | 2608080CB1 |
| 7503402 | 3 | 7503402CD1 | 61 | 7503402CB1 |
| 7503517 | 4 | 7503517CD1 | 62 | 7503517CB1 |
| 7500014 | 5 | 7500014CD1 | 63 | 7500014CB1 |
| 7501365 | 6 | 7501365CD1 | 64 | 7501365CB1 |
| 7503540 | 7 | 7503540CD1 | 65 | 7503540CB1 |
| 7504326 | 8 | 7504326CD1 | 66 | 7504326CB1 |
| 7504388 | 9 | 7504388CD1 | 67 | 7504388CB1 |
| 2828380 | 10 | 2828380CD1 | 68 | 2828380CB1 |
| 6456919 | 11 | 6456919CD1 | 69 | 6456919CB1 |
| 7502244 | 12 | 7502244CD1 | 70 | 7502244CB1 |
| 7498718 | 13 | 7498718CD1 | 71 | 7498718CB1 |
| 6259308 | 14 | 6259308CD1 | 72 | 6259308CB1 |
| 7504104 | 15 | 7504104CD1 | 73 | 7504104CB1 |
| 7504121 | 16 | 7504121CD1 | 74 | 7504121CB1 |
| 5635695 | 17 | 5635695CD1 | 75 | 5635695CB1 |
| 7503983 | 18 | 7503983CD1 | 76 | 7503983CB1 |
| 7503476 | 19 | 7503476CD1 | 77 | 7503476CB1 |
| 7504023 | 20 | 7504023CD1 | 78 | 7504023CB1 |
| 7504128 | 21 | 7504128CD1 | 79 | 7504128CB1 |
| 4529338 | 22 | 4529338CD1 | 80 | 4529338CB1 |
| 7503460 | 23 | 7503460CD1 | 81 | 7503460CB1 |
| 5466630 | 24 | 5466630CD1 | 82 | 5466630CB1 |
| 7503474 | 25 | 7503474CD1 | 83 | 7503474CB1 |
| 7503498 | 26 | 7503498CD1 | 84 | 7503498CB1 |
| 7504119 | 27 | 7504119CD1 | 85 | 7504119CB1 |
| 71532805 | 28 | 71532805CD1 | 86 | 71532805CB1 |
| 5502992 | 29 | 5502992CD1 | 87 | 5502992CB1 |
| 7503828 | 30 | 7503828CD1 | 88 | 7503828CB1 |
| 2647325 | 31 | 2647325CD1 | 89 | 2647325CB1 |
| 7495416 | 32 | 7495416CD1 | 90 | 7495416CB1 |
| 8096177 | 33 | 8096177CD1 | 91 | 8096177CB1 |
| 666763 | 34 | 666763CD1 | 92 | 666763CB1 |
| 7504091 | 35 | 7504091CD1 | 93 | 7504091CB1 |
| 7503568 | 36 | 7503568CD1 | 94 | 7503568CB1 |
| 7504101 | 37 | 7504101CD1 | 95 | 7504101CB1 |
| 6946680 | 38 | 6946680CD1 | 96 | 6946680CB1 |
| 7001142 | 39 | 7001142CD1 | 97 | 7001142CB1 |
| 71158380 | 40 | 71158380CD1 | 98 | 71158380CB1 |
| 7503861 | 41 | 7503861CD1 | 99 | 7503861CB1 |
| 7758395 | 42 | 7758395CD1 | 100 | 7758395CB1 |
| 71039312 | 43 | 71039312CD1 | 101 | 71039312CB1 |
| 7291318 | 44 | 7291318CD1 | 102 | 7291318CB1 |
| 2638619 | 45 | 2638619CD1 | 103 | 2638619CB1 |
| 2810014 | 46 | 2810014CD1 | 104 | 2810014CB1 |
| 3457155 | 47 | 3457155CD1 | 105 | 3457155CB1 |
| 7435171 | 48 | 7435171CD1 | 106 | 7435171CB1 |
| 7499936 | 49 | 7499936CD1 | 107 | 7499936CB1 |
| 7504125 | 50 | 7504125CD1 | 108 | 7504125CB1 |
| 7505742 | 51 | 7505742CD1 | 109 | 7505742CB1 |
| 7505757 | 52 | 7505757CD1 | 110 | 7505757CB1 |
| 7504126 | 53 | 7504126CD1 | 111 | 7504126CB1 |
| 7504099 | 54 | 7504099CD1 | 112 | 7504099CB1 |
| 7505733 | 55 | 7505733CD1 | 113 | 7505733CB1 |
| 7959829 | 56 | 7959829CD1 | 114 | 7959829CB1 |
| 7502168 | 57 | 7502168CD1 | 115 | 7502168CB1 |
| 7503888 | 58 | 7503888CD1 | 116 | 7503888CB1 |

| | | | | Incyte | |
|---|---|---|---|---|---|
| | Polypeptide | Incyte | Polynucleotide | Polynucleotide | |
| Incyte Project ID | SEQ ID NO: | Polypeptide ID | SEQ ID NO: | ID | Incyte Full Length Clones |
| 7503848 | 1 | 7503848CD1 | 59 | 7503848CB1 | |
| 2608080 | 2 | 2608080CD1 | 60 | 2608080CB1 | |
| 7503402 | 3 | 7503402CD1 | 61 | 7503402CB1 | 6308169CA2 |
| 7503517 | 4 | 7503517CD1 | 62 | 7503517CB1 | |
| 7500014 | 5 | 7500014CD1 | 63 | 7500014CB1 | 90040096CA2, 90045149CA2, 90045157CA2, 90045165CA2, |
| | | | | | 90045181CA2, 90045189CA2, 90045201CA2, 90045233CA2, |
| | | | | | 90045249CA2, 90045265CA2, 90045273CA2, 90045281CA2, |
| | | | | | 90045289CA2, 90166707CA2, 90166739CA2, 90166815CA2, |
| | | | | | 90166831CA2 |
| 7501365 | 6 | 7501365CD1 | 64 | 7501365CB1 | |
| 7503540 | 7 | 7503540CD1 | 65 | 7503540CB1 | |
| 7504326 | 8 | 7504326CD1 | 66 | 7504326CB1 | |
| 7504388 | 9 | 7504388CD1 | 67 | 7504388CB1 | |
| 2828380 | 10 | 2828380CD1 | 68 | 2828380CB1 | |
| 6456919 | 11 | 6456919CD1 | 69 | 6456919CB1 | 3212008CA2 |
| 7502244 | 12 | 7502244CD1 | 70 | 7502244CB1 | |
| 7498718 | 13 | 7498718CD1 | 71 | 7498718CB1 | |
| 6259308 | 14 | 6259308CD1 | 72 | 6259308CB1 | 8653345CA2 |
| 7504104 | 15 | 7504104CD1 | 73 | 7504104CB1 | 2654926CA2 |
| 7504121 | 16 | 7504121CD1 | 74 | 7504121CB1 | |
| 5635695 | 17 | 5635695CD1 | 75 | 5635695CB1 | |
| 7503983 | 18 | 7503983CD1 | 76 | 7503983CB1 | 2215488CA2, 8662527CA2 |
| 7503476 | 19 | 7503476CD1 | 77 | 7503476CB1 | |
| 7504023 | 20 | 7504023CD1 | 78 | 7504023CB1 | |
| 7504128 | 21 | 7504128CD1 | 79 | 7504128CB1 | |
| 4529338 | 22 | 4529338CD1 | 80 | 4529338CB1 | |
| 7503460 | 23 | 7503460CD1 | 81 | 7503460CB1 | 90062547CA2, 90062615CA2, 90062623CA2, 90062639CA2 |
| 5466630 | 24 | 5466630CD1 | 82 | 5466630CB1 | |
| 7503474 | 25 | 7503474CD1 | 83 | 7503474CB1 | |
| 7503498 | 26 | 7503498CD1 | 84 | 7503498CB1 | 2170945CA2 |
| 7504119 | 27 | 7504119CD1 | 85 | 7504119CB1 | 95135029CA2 |
| 71532805 | 28 | 71532805CD1 | 86 | 71532805CB1 | |
| 5502992 | 29 | 5502992CD1 | 87 | 5502992CB1 | |
| 7503828 | 30 | 7503828CD1 | 88 | 7503828CB1 | |
| 2647325 | 31 | 2647325CD1 | 89 | 2647325CB1 | 90177208CA2 |
| 7495416 | 32 | 7495416CD1 | 90 | 7495416CB1 | |
| 8096177 | 33 | 8096177CD1 | 91 | 8096177CB1 | 90170506CA2 |
| 666763 | 34 | 666763CD1 | 92 | 666763CB1 | |
| 7504091 | 35 | 7504091CD1 | 93 | 7504091CB1 | |
| 7503568 | 36 | 7503568CD1 | 94 | 7503568CB1 | |
| 7504101 | 37 | 7504101CD1 | 95 | 7504101CB1 | |
| 6946680 | 38 | 6946680CD1 | 96 | 6946680CB1 | |
| 7001142 | 39 | 7001142CD1 | 97 | 7001142CB1 | 90180809CA2 |
| 71158380 | 40 | 71158380CD1 | 98 | 71158380CB1 | 4913234CA2 |
| 7503861 | 41 | 7503861CD1 | 99 | 7503861CB1 | |
| 7758395 | 42 | 7758395CD1 | 100 | 7758395CB1 | |
| 71039312 | 43 | 71039312CD1 | 101 | 71039312CB1 | |
| 7291318 | 44 | 7291318CD1 | 102 | 7291318CB1 | |
| 2638619 | 45 | 2638619CD1 | 103 | 2638619CB1 | |
| 2810014 | 46 | 2810014CD1 | 104 | 2810014CB1 | 3387728CA2, 90166951CA2, 90166967CA2, 90166975CA2, |
| | | | | | 90166983CA2, 90166991CA2, 90167051CA2, 90167067CA2 |
| 3457155 | 47 | 3457155CD1 | 105 | 3457155CB1 | |
| 7435171 | 48 | 7435171CD1 | 106 | 7435171CB1 | |
| 7499936 | 49 | 7499936CD1 | 107 | 7499936CB1 | 90041227CA2, 90041243CA2, 90041319CA2 |
| 7504125 | 50 | 7504125CD1 | 108 | 7504125CB1 | 90057593CA2, 90057785CA2, 90057853CA2, 90057955CA2, |
| | | | | | 90057963CA2, 90057971CA2, 90057979CA2, 90057987CA2, |
| | | | | | 90057995CA2, 90058033CA2, 90058055CA2, 90058063CA2, |
| | | | | | 90058071CA2, 90058079CA2, 90058087CA2, 90058095CA2 |
| 7505742 | 51 | 7505742CD1 | 109 | 7505742CB1 | |
| 7505757 | 52 | 7505757CD1 | 110 | 7505757CB1 | |
| 7504126 | 53 | 7504126CD1 | 111 | 7504126CB1 | 4549855CA2 |
| 7504099 | 54 | 7504099CD1 | 112 | 7504099CB1 | |
| 7505733 | 55 | 7505733CD1 | 113 | 7505733CB1 | |
| 7959829 | 56 | 7959829CD1 | 114 | 7959829CB1 | 4111545CA2, 90176769CA2, 90176777CA2, 90176785CA2, |
| | | | | | 90176853CA2, 90176861CA2, 90176869CA2 |
| 7502168 | 57 | 7502168CD1 | 115 | 7502168CB1 | |
| 7503888 | 58 | 7503888CD1 | 116 | 7503888CB1 | |

TABLE 2

| Poly- | | | | |
|---|---|---|---|---|
| peptide | | GenBank ID NO: | | |
| SEQ | Incyte | or PROTEOME | Probability | |
| ID NO: | Polypeptide ID | ID NO: | Score | Annotation |
| 1 | 7503848CD1 | g1854952 | 0.0 | [Homo sapiens] putative nucleolar trafficking phosphoprotein |
| | | | | Wise, C. A. et al. (1997) TCOF1 gene encodes a putative nucleolar |
| | | | | phosphoprotein that exhibits mutations in Treacher Collins Syndrome throughout |
| | | | | its coding region. Proc. Natl. Acad. Sci. U.S.A. 94: 3110-3115 |
| | | 338442\|TCOF1 | 0.0 | [Homo sapiens][Nuclear import/exportprotein; Transporter] [Nuclear |
| | | | | nucleolus; Nuclear] Treacle, protein with similarity to nucleolar trafficking |
| | | | | proteins that isphosphorylated by casein kinase; mutation of corresponding |
| | | | | genecauses Treacher Collins Syndrome |
| | | 320096\|Tcof1 | 2.7E−211 | [Mus musculus][Nuclear import/export protein] [Nuclear nucleolus; Nuclear] |
| | | | | Protein with similarity to nucleolar phosphoproteins, may have a role in nucleolar- |
| | | | | cytoplasmic transportand craniofacial development; putative human ortholog |
| | | | | TCOF1 is associated with Treacher Collins Syndrome |
| | | 239850\|C25A1.10 | 6.1E−41 | [Caenorhabditis elegans][Nuclear import/exportprotein][Nuclear pore] Putative |
| | | | | nucleoporin, has moderate similarity to H. sapeins P130 gene product [nucleolar |
| | | | | phosphoprotein p130] |
| | | 247598\|K06A9.1 | 2.4E−35 | [Caenorhabditis elegans] Putative mucin, has strong similarity to H. sapiens |
| | | | | MUC1 gene product [mucin 1, transmembrane] |
| | | 630082\|orf6.162 | 2.8E−31 | [Candida albicans] Protein of unknown function, has a region of low similarity to |
| | | | | C. albicans Hwp1p, which is a hyphal-specific cell wall protein with a role in |
| | | | | attachment to host epithelial cells |
| 2 | 2608080CD1 | g1020145 | 1.1E−149 | [Homo sapiens] DNA binding protein |
| | | | | Bellefroid, E. J. et al. (1989) The human genome contains hundreds of genes |
| | | | | coding for finger proteins of the Kruppel type. DNA 8: 377-387 |
| | | 346272\|ZNF264 | 3.7E−182 | [Homo sapiens][Inhibitor or repressor; Transcription factor] Protein with high |
| | | | | similarity to ZNF184, which is a KRAB zinc finger protein that is expressed in |
| | | | | testis, contains a KRAB (kruppel-associated box) domain, which may mediate |
| | | | | transcriptional repression, and twelve C2H2 type zinc finger domains |
| | | 339004\|ZNF84 | 9.4E−151 | [Homo sapiens][Inhibitor or repressor; DNA-binding protein; Transcription |
| | | | | factor] [Nuclear] Protein containing a KRAB (kruppel-associated box) domain |
| | | | | which may mediate transcriptional repression and several C2H2 type zinc finger |
| | | | | domains, which bind nucleic acids |
| | | 308339\|ZNF184 | 2.7E−149 | [Homo sapiens] Kruppel-like zinc-finger protein, maximally expressed in testis, |
| | | | | moderately in other tissues |
| | | 339006\|ZNF85 | 2.9E−145 | [Homo sapiens][Inhibitor or repressor; Transcription factor; DNA-binding protein] |
| | | | | [Nuclear] Zinc-finger transcriptional repressor containing a Kruppel-associated |
| | | | | box (KRAB) domain, member of the ZNF91 family of zinc-finger proteins |
| | | 339008\|ZNF91 | 7.8E−145 | [Homo sapiens] Zinc-finger protein containing a Kruppel-associated box (KRAB) |
| | | | | transcriptional repression domain, most highly expressed in T lymphoid cells and |
| | | | | down-regulated during in vitro terminal differentiation of myeloid cells |
| 3 | 7503402CD1 | g495572 | 0.0 | [Homo sapiens] zinc finger protein |
| | | | | Tommerup, N. and Vissing, H. (1995) Isolation and fine mapping of 16 novel |
| | | | | human zinc finger-encoding cDNAs identify putative candidate genes for |
| | | | | developmental and malignant disorders. Genomics 27: 259-264 |
| | | 338964\|ZNF143 | 0.0 | [Homo sapiens] [Activator; DNA-binding protein; Transcription factor] Zinc- |
| | | | | finger transcriptional activator of small nuclear (snRNA) and snRNA-type genes |
| | | | | transcribed by RNA polymerases II and III |
| | | 324316\|D7Ertd805e | 0.0 | [Mus musculus][Activator; DNA-binding protein; Transcription factor] Zinc- |
| | | | | finger transcriptional activator of the selenocysteine tRNA (tRNAsec), binding |
| | | | | activity in mammary glands increases in parallel with the increase of tRNAsec |
| | | | | transcript during the periods of pregnancy and lactation |
| | | 339000\|ZNF76 | 1.9E−130 | [Homo sapiens][Activator; Transcription factor; DNA-binding protein] Kruppel- |
| | | | | like zinc-finger transcriptional activator of the small nuclear (snRNA) and snRNA- |
| | | | | type genes transcribed by RNA polymerases II and III, expressed in testis |
| | | 324156\|Mm.10509 | 1.4E−54 | [Mus musculus][DNA-binding protein][Nuclear] Protein containing a C2H2 type |
| | | | | zinc finger domain, which bind nucleic acids |
| | | 432838\|ZNF180 | 1.6E−54 | [Homo sapiens] Zinc finger protein; corresponding gene is localized in a region |
| | | | | associated with rearrangements leading to developmental abnormalities, DNA |
| | | | | repair deficiencies, and cellular malignancies |
| 4 | 7503517CD1 | g9651997 | 4.7E−219 | [Homo sapiens] eukaryotic translation initiation factor EIF2B subunit 3 |
| | | | | Kruger, M. et al. (2000) Identification of eIF2B gamma and eIF2 gamma as |
| | | | | cofactors of hepatitis C virus internal ribosome entry site-mediated translation |
| | | | | using a functional genomics approach. Proc. Natl. Acad. Sci. U.S.A. 97: 8566-8571 |
| | | 610840\|EIF2B3 | 4.1E−220 | [Homo sapiens][Translation factor][Cytoplasmic] Subunit of eukaryotic |
| | | | | translation initiation factor 2B |
| | | 330762\|Rn.10577 | 1.9E−200 | [Rattus norvegicus][Guanine nucleotide exchange actor; Translation |
| | | | | factor][Cytoplasmic] Gamma subunit of translation initiation factor 2B, a |
| | | | | heteropentamer that mediates the exchange of GDP bound to translation initiation |
| | | | | factor eIF2 for GTP |
| | | 439325\|ppp-1 | 8.8E−39 | [Caenorhabditis elegans] [Transferase; Translation factor] [Cytoplasmic] Protein |
| | | | | containing a putative NTP transferase (nucleotidyl transferase) domain, has weak |
| | | | | similarity to S. cerevisiae Psa1p (mannose-1-phosphate guanyltransferase; GDP- |
| | | | | mannose pyrophosphorylase) |
| | | 370068\|tif223 | 1.1E−33 | [Schizosaccharomyces pombe] [Translation factor] Putative translation initiation |
| | | | | factor eIF-2b gamma subunit, has low similarity to S. cerevisiae Gcd1p |
| | | 643938\|orf6.7090 | 6.8E−20 | [Candida albicans][Guanine nucleotide exchange factor; Translation factor] |
| | | | | Protein containing three bacterial transferase hexapeptide (four repeats) domains, |
| | | | | has low similarityto S. cerevisiae Gcd1p, which is a translation initiation factor |
| | | | | eIF2B |
| 5 | 7500014CD1 | g12654757 | 1.6E−55 | [Homo sapiens] nuclear receptor binding protein |
| | | 432864\|NRBP | 1.4E−56 | [Homo sapiens][Nuclear] Adaptor protein with two nuclear receptor binding |
| | | | | motifs, a SH2 binding domain, a kinase-like domain and a nuclear localization |
| | | | | signal, may have a role in the signaling pathways involving nuclear receptors and |
| | | | | SH2 domain containing proteins |
| 6 | 7501365CD1 | g11322247 | 3.9E−210 | [Homo sapiens] nucleolar protein No55 |
| | | 343772\|SC65 | 9.2E−211 | [Homo sapiens][Nuclear nucleolus; Nuclear] Nucleolar protein that associates with |
| | | | | chromosomes during mitosis and has similarityto rat SC65 (Rn.40377), a |
| | | | | synaptonemal complex protein |
| | | 333646\|Sc65 | 2.0E−176 | [Rattus norvegicus][DNA-binding protein][Nuclear] Component of synaptonemal |
| | | | | complex localized between paired aligned cores of homologous chromosomes |
| | | 609086\|Crtap | 1.3E−110 | [Mus musculus] Cartilage associated protein, a protein that is expressed in |
| | | | | embryonic cartilage |
| | | 343398\|CRTAP | 8.9E−110 | [Homo sapiens] Cartilage associated protein, has strong similarity to murine |
| | | | | Crtap, which is a protein that is expressed in embryonic cartilage |
| | | 613744\|Gros1 | 7.5E−50 | [Mus musculus] [Inhibitor or repressor] Growth suppressor, expression in cell |
| | | | | culture results in slow growth of cells and reduced colony-formation |
| 7 | 7503540CD1 | g5734605 | 0.0 | [Homo sapiens] KARP-1-binding protein 3 |
| | | 346328\|KIAA0470 | 0.0 | [Homo sapiens] Protein containing a forkhead associated (FHA), which bind |
| | | | | phosphotyrosine residues |
| | | 434396\|KIAA0284 | 1.5E−125 | [Homo sapiens] Protein of unknown function, has a region of low similarity to a |
| | | | | region of rat Rn.32072, which is a salivary protein belonging to a proline-rich |
| | | | | protein family that also includes RP13 (Rn.9841) and RP15 (Rn.9842) |
| | | 4988\|MUC1 | 6.9E−12 | [Saccharomyces cerevisiae] [Hydrolase] [Cell wall] Cell surfaceflocculin, |
| | | | | required for invasive and pseudohyphal growth |
| 8 | 7504326CD1 | g14915787 | 0.0 | [Mus musculus] WAC |
| | | 4988\|MUC1 | 1.9E−15 | [Saccharomyces cerevisiae] [Hydrolase] [Cell wall] Cell surface flocculin, |
| | | | | required for invasive and pseudohyphal growth |
| | | 370430\|SPBC215.13 | 3.5E−11 | [Schizosaccharomyces pombe] Serine-rich protein |
| 9 | 7504388CD1 | g14009498 | 4.8E−86 | [Homo sapiens] hairy/enhancer of split 6 |
| | | | | Vasiliauskas, D. and Stern, C. D. (2000) Expression of mouse HES-6, a new |
| | | | | member of the Hairy/Enhancer of split family of bHLH transcription factors. |
| | | | | Mech. Dev. 98: 133-137 |
| | | 599700\|HES6 | 1.4E−100 | [Homo sapiens][Inhibitor or repressor; Transcription factor] Basic helix-loop- |
| | | | | helix protein, does not bind DNA but acts as an inhibitor of Hes1 and suppresses |
| | | | | Hes1 from repressing transcription |
| | | 608436\|Hes6 | 4.5E−85 | [Mus musculus] Member of the family of homologs of Drosophila hairy and |
| | | | | Enhancer of split, a basic helix-loop-helix protein that inhibits the transcriptional |
| | | | | repressor Hes1 and promotes cell differentiation |
| | | 321888\|Hes1 | 5.7E−14 | [Mus musculus][Inhibitor or repressor; DNA-binding protein; Transcription |
| | | | | factor] Hairy and enhancer of split, a helix-loop-helix negative regulator of |
| | | | | transcription |
| | | 344428\|HRY | 7.3E−14 | [Homo sapiens][DNA-binding protein] Homolog of Drosophila hairy, has very |
| | | | | strong similarity to murine Hes1, which is a helix-loop-helix negative regulator of |
| | | | | transcription, has very strong similarity to rat Rn.19727, which suppresses |
| | | | | neuronal differentiation |
| | | 688984\|Hes1 | 9.8E−14 | [Rattus norvegicus] [Inhibitor or repressor; DNA-binding protein; Transcription |
| | | | | factor] [Nuclear] Hairy-like, transduces growth factor signals during embryonic |
| | | | | development |
| 10 | 2828380CD1 | g13752754 | 8.5E−240 | [Homo sapiens] zinc finger 1111 |
| | | 339008\|ZNF91 | 9.9E−230 | [Homo sapiens] Zinc-finger protein 91 (HPF7, HTF10), member of KRAB |
| | | | | subfamily of C2H2 zinc finger proteins, functions as a transcriptional repressor, |
| | | | | may play a role in formation of seminomas, down-regulated during in vitro |
| | | | | myeloid cell differentiation |
| | | 691254\|FLJ14345 | 6.6E−217 | [Homo sapiens] Protein with high similarity to human ZNF255, which is kruppel- |
| | | | | like zinc finger protein that may activate transcription |
| | | 432896\|ZNF208 | 2.6E−213 | [Homo sapiens] Zinc finger protein 208, a ubiquitously expressed Kruppel- |
| | | | | associated box (KRAB) zinc finger protein |
| | | 338994\|ZNF43 | 2.3E−205 | [Homo sapiens] Zinc finger protein 43, contains C2H2 zinc finger motifs, |
| | | | | expressed mainly in B and T cells |
| | | 365207\|ZNF197 | 1.1E−200 | [Homo sapiens][Transcription factor] Zinc finger protein 197, member of the zinc- |
| | | | | finger transcription factor family, contains twenty C2H2-type zinc finger motifs, |
| | | | | high level expressionis associated with thyroid papillary carcinomas |
| 11 | 6456919CD1 | g930123 | 1.7E−147 | [Homo sapiens] zinc finger protein (583 AA) |
| | | 435298\|ZNF20 | 7.3E−163 | [Homo sapiens][DNA-binding protein; Transcription factor; Small molecule- |
| | | | | binding protein] Putative DNA-binding protein with a zinc finger motif |
| | | 594469\|HSZFP36 | 1.5E−148 | [Homo sapiens][Inhibitor or repressor; Transcription factor; DNA-binding protein] |
| | | | | Protein containing fourteen C2H2 type zinc finger domains, which bind nucleic |
| | | | | acids, also contains a KRAB (kruppel-associated box) domain which may mediate |
| | | | | transcriptional repression |
| | | 623668\|ZNF14 | 3.1E−146 | [Homo sapiens] Zinc finger protein isolated from cell lines of T-cell origin |
| | | 476345\|LOC51712 | 3.1E−146 | [Homo sapiens][Inhibitor or repressor; Transcription factor; DNA-binding protein] |
| | | | | Protein containing eighteen C2H2 type zinc finger domains, which bind nucleic |
| | | | | acids, also contains a KRAB (kruppel-associated box) domain which may mediate |
| | | | | transcriptional repression |
| | | 338956\|ZNF136 | 7.5E−145 | [Homo sapiens][Inhibitor or repressor; Transcriptionfactor; DNA-binding protein] |
| | | | | C2H2 zinc-finger protein containing a Kruppel-associated box-A (KRAB-A) |
| | | | | transcriptional repression domain, represses transcription when fused to the |
| | | | | heterologous KRABB subdomain of human ZNF10 |
| 13 | 7498718CD1 | g2897601 | 7.4E−170 | [Homo sapiens] kruppel-type zinc finger protein |
| | | | | Blin, N. (1997) Expressed sequences within pericentromeric heterochromatin of |
| | | | | human chromosome 22. Mamm. Genome 8: 859-862 |
| | | 704009\|ZNF73 | 6.4E−171 | [Homo sapiens] Member of the Kruppel type family of zinc finger proteins |
| | | 338968\|ZNF157 | 1.0E−123 | [Homo sapiens][Inhibitor or repressor; Transcription factor] Zinc finger protein |
| | | | | 157, a zinc-finger protein that contains two Kruppel-associated box (KRAB-A and |
| | | | | KRAB-B) transcription repression domains |
| | | 308339\|ZNF184 | 4.5E−123 | [Homo sapiens] Kruppel-like zinc-finger protein, maximally expressed in testis, |
| | | | | moderately in other tissues |
| | | 435075\|ZNF41 | 1.4E−121 | [Homo sapiens][Inhibitor or repressor; Transcription factor] Zinc finger protein |
| | | | | with 18 contiguous zinc fingers of the C2H2 type, contains a KRAB/FPB |
| | | | | (Kruppel-associated/finger preceding box) domain, which probably functions in |
| | | | | transcriptional repression |
| | | 587437\|Zfp68 | 7.6E−120 | [Mus musculus][Inhibitor or repressor; Transcription factor] KRAB-containing |
| | | | | zinc-finger protein that when bound to the corepressor KAP-1, forms a functional |
| | | | | transcriptional repressor complex |
| 14 | 6259308CD1 | g1916290 | 4.0E−130 | [Mus musculus] ALY |
| | | | | Bruhn, L. (1997) ALY, a context-dependent coactivator of LEF-1 and AML-1, is |
| | | | | required for TCRalpha enhancer function. Genes Dev. 11: 640-653 |
| | | 585985\|Refbp1 | 3.5E−131 | [Mus musculus][Transcription factor; RNA-binding protein] |
| | | | | [Nuclear; Cytoplasmic] Member of the T-cell receptor alpha (TCR alpha) enhancer |
| | | | | complex that interacts with the activation domains of LEF-1 and AML-1 to |
| | | | | stimulate transcription from theT-cell receptor alpha (TCR alpha) enhancer |
| | | 348154\|ALY | 1.1E−120 | [Homo sapiens][Activator; DNA-binding protein; Transcriptionfactor; RNA- |
| | | | | binding protein] [Nuclear] Ortholog of murine Mm. 1886, a member of the T-cell |
| | | | | receptor alpha (TCR alpha) enhancer complex that acts to stimulate transcription |
| | | | | from the T-cell receptor alpha (TCR alpha) enhancer, may have a role in systemic |
| | | | | lupus erythematosus |
| | | 597373\|Refbp2 | 1.6E−99 | [Mus musculus][RNA-binding protein] [Nuclear] RNA and expor tactor binding |
| | | | | protein 2, member of a conserved family of heterogeneous nuclear |
| | | | | ribonucleoprotein-like proteins which binds nuclear RNA and has a role in mRNA |
| | | | | export from the nucleus, contains an RNA recognition motif (RRM) domain |
| | | 243563\|F23B2.6 | 2.9E−19 | [Caenorhabditis elegans][RNA-binding protein] Member of the RRM domain |
| | | | | protein family |
| | | 239659\|C18D11.4 | 3.0E−16 | [Caenorhabditis elegans][RNA-binding protein] [Nuclear] Protein with strong |
| | | | | similarity to human SFRS10 protein and SR-like splicing factor and Drosophila |
| | | | | TRA2, (putative RNA binding protein) |
| 15 | 7504104CD1 | g338013 | 4.5E−266 | [Homo sapiens] SEF2-1A protein |
| | | | | Corneliussen, B. (1991) Helix-loop-helix transcriptional activators bind to a |
| | | | | sequence in glucocorticoid response elements of retrovirus enhancers. J. Virol. |
| | | | | 65: 6084-6093 |
| | | 338432\|TCF4 | 1.2E−263 | [Homo sapiens][Activator; DNA-binding protein; Transcription factor] [Nuclear] |
| | | | | Transcription factor 4, basic helix-loop-helix transcriptional co-activator and |
| | | | | repressor, plays a role in the Wnt signaling pathway; mutations in the |
| | | | | corresponding gene are associated with colorectal tumors |
| | | 587379\|Tcf4 | 5.3E−263 | [Mus musculus][Activator; Inhibitor or repressor; DNA-binding |
| | | | | protein; Transcription factor; Small molecule-binding protein] Transcription factor |
| | | | | 4, basic helix-loop-helix transcriptional co-activator and co-repressor, plays a role |
| | | | | in the Wnt signaling pathway and is essential for normal gastrulation; mutations in |
| | | | | the human TCF4 gene are associated with colorectal tumors |
| | | 330540\|Rn.10450 | 2.1E−229 | [Rattus norvegicus][Activator; DNA-binding protein; Transcription factor] |
| | | | | [Nuclear] Transcription factor 4, hepatocyte nuclear factor 4 alpha, basic helix- |
| | | | | loop-helix transcriptional co-activator and repressor, activates beta-cell genes |
| | | | | involved in glucose metabolism; mutations in the human TCF4 gene are |
| | | | | associated with colorectal tumors |
| | | 339804\|TCF12 | 7.8E−166 | [Homo sapiens][Activator; DNA-binding protein; Transcription factor] Basic helix- |
| | | | | loop-helix (bHLH) transcriptional activator that binds to the immunoglobulin |
| | | | | enhancer E-box consensus sequence, forms complexes with the immunoglobulin |
| | | | | enhancer binding proteins E12 and ITF2 and the myogenic factor myogenin |
| | | | | (MYOG) |
| | | 330280\|Rn.10290 | 2.1E−91 | [Rattus norvegicus][Activator; Transcription factor; DNA-binding protein] |
| | | | | Transcriptional activator with similarity to E12 and E47, may be involved in the |
| | | | | regulation of pancreatic exocrine genes, including insulin and chymotrypsin |
| 16 | 7504121CD1 | g3258665 | 9.8E−191 | [Gallus gallus] transcription factor LEF-1 |
| | | | | Kengaku, M. (1998) Distinct WNT pathways regulating AER formation and |
| | | | | dorsoventral polarity in the chick limb bud. Science 280: 1274-1277 |
| | 7504121CD1 | 625410\|LEF1 | 7.6E−120 | [Homo sapiens][Activator; Inhibitor or repressor; DNA-binding |
| | | | | protein; Transcription factor] [Nuclear] Protein with very strong similarity to |
| | | | | murine Lef1, which is a member of the HMG-box family of transcription factors |
| | | | | that activates transcription of T-cell receptor alpha (TCRA), and which may |
| | | | | regulate lymphocyte gene expression and differentiation |
| | | 585197\|Lef1 | 6.6E−82 | [Mus musculus][Activator; DNA-binding protein; Transcription factor] [Nuclear] |
| | | | | Lymphoid enhancer binding factor 1, member of the HMG-box family of |
| | | | | transcription factors, activates transcription of T-cell receptor alpha (TCRA), may |
| | | | | be a regulator of lymphocyte gene expression and differentiation |
| | | 338436\|TCF7 | 3.8E−63 | [Homo sapiens][Activator; DNA-binding protein; Transcription factor] |
| | | | | Transcription factor 7, transcriptional activator that binds to T cell-specific |
| | | | | elements and plays a role in T cell differentiation; may be associated with late |
| | | | | events in colorectal cell tumor progression |
| | | 429226\|Tcf7 | 2.7E−60 | [Mus musculus][Activator; DNA-binding protein; Transcription factor] |
| | | | | Transcription factor 7, transcriptional activator that binds to T cell-specific |
| | | | | elements and plays a role in T cell differentiation; human TCF7 may be associated |
| | | | | with late events in colorectal cell tumor progression |
| | | 429228\|Tcf712 | 2.8E−58 | [Mus musculus][Activator; DNA-binding protein; Transcription factor] [Nuclear] |
| | | | | HMG-box transcriptional activator, forms a complex with beta-catenin (Catnb) or |
| | | | | Armadillo that stimulates transcription in response to Wnt/Wingless signaling, |
| | | | | may be involved in gastrointestinal tract development |
| 17 | 5635695CD1 | g14333988 | 0.0 | [Homo sapiens] enhancer of polycomb 1 |
| | | 697396\|EPC1 | 2.8E−200 | [Homo sapiens] Enhancer of polycomb, both represses and activates transcription |
| | | 325698\|Epc1 | 1.2E−192 | [Mus musculus] Protein with similarity to the Drosophila enhancer of polycomb |
| | | | | E(PC) gene, may regulate chromatin structure |
| | | 256495\|Y111B2 | 5.3E−51 | [Caenorhabditis elegans] Protein with strong similarity to D. melanogaster E(Pc) |
| | | A.I | | (Enhancer of Polycomb) protein |
| 18 | 7503983CD1 | g15215451 | 4.8E−132 | [Homo sapiens] (BC012819) eukaryotic translation elongation factor 1 delta |
| | | | | (guanine nucleotide exchange protein) |
| | | 742632\|FLJ20897 | 4.3E−129 | [Homo sapiens] Translation elongation factor 1 delta, a guanine-nucleotide |
| | | | | exchange protein that contains a leucine zipper motif |
| | | 742436\|EEF1B2 | 2.3E−56 | [Homo sapiens] Eukaryotic translation elongation factor 1beta 2, putative |
| | | | | component of the eukaryotic translation elongation complex |
| | | 608120\|Eef1b2 | 1.6E−55 | [Mus musculus] [Guanine nucleotide exchange factor; Translation factor] |
| | | | | [Cytoplasmic] Protein with very strong similarity to human EEF1B2, eukaryotic |
| | | | | translation elongation factor 1 beta 2, a putative component of the eukaryotic |
| | | | | translation elongation complex |
| | | 276349\|F54H12.6 | 3.3E−46 | [Caenorhabditis elegans] Member of the elongation factor 1 (beta/delta chain) |
| | | | | protein family |
| | | 252376\|Y41E3.10 | 3.4E−46 | [Caenorhabditis elegans] [Translation factor] [Cytoplasmic] Putative translation |
| | | | | elongation factor 1[beta/delta chain] |
| 19 | 7503476CD1 | g550017 | 1.4E−31 | [Homo sapiens] ribosomal protein L27a |
| | | 337714\|RPL27A | 1.2E−32 | [Homo sapiens] [Structural protein; RNA-binding protein; Ribosomal subunit] |
| | | | | [Cytoplasmic] Ribosomal protein L27a, component of the large 60S ribosomal |
| | | | | subunit; gene is abnormally expressed in colorectal carcinomas |
| | | | | Belhumeur, P. et al. (1987) Nucleic Acids Res 15: 1019-1029 |
| | | | | Isolation and characterisation of a murine cDNA clone highly homologous to the |
| | | | | yeast L29 ribosomal protein gene. |
| | | 674449\|Rpl27a | 8.8E−32 | [Mus musculus] [Structural protein; RNA-binding protein; Ribosomal subunit] |
| | | | | [Cytoplasmic] Ribosomal protein L27a, component of the large 60S ribosomal |
| | | | | subunit; human RPL27A is abnormally expressed in colorectal carcinomas |
| | | 6726\|RPL28 | 1.2E−18 | [Saccharomyces cerevisiae] [RNA-binding protein; Ribosomal subunit] |
| | | | | [Nuclear; Cytoplasmic] Ribosomal protein L28 (yeast L29; YL24; rp44; mouse and |
| | | | | rat L27a) |
| | | 371142\|rpl28-2 | 6.7E−18 | [Schizosaccharomyces pombe] [Ribosomal subunit] 60S ribosomal protein |
| | | | | L28B/L27a/L29 |
| | | 376062\|rpl28-1 | 1.1E−17 | [Schizosaccharomyces pombe] [Ribosomal subunit] 60S ribosomal protein L28 |
| 20 | 7504023CD1 | g12653155 | 9.3E−102 | [Homo sapiens] ribosomal protein, large, P0 |
| | | 337756\|RPLP0 | 8.1E−103 | [Homo sapiens] [Structural protein; RNA-binding protein; Ribosomal subunit] |
| | | | | [Cytoplasmic] Ribosomal protein P0, acidic phosphoprotein component of the |
| | | | | large 60S ribosomal subunit; shows increased expression in hepatocellular and |
| | | | | colon carcinomas |
| | | | | Krowczynska, A. M. et al. (1989) Nucleic Acids Res. 17: 6408 |
| | | | | The mouse homologue of the human acidic ribosomal phosphoprotein PO: a |
| | | | | highly conserved polypeptide that is under translational control. |
| | | 327804\|Rn.1079 | 2.1E−102 | [Rattus norvegicus] [Structural protein; RNA-binding protein; Ribosomal subunit] |
| | | | | [Cytoplasmic] Ribosomal protein P0, acidic phosphoprotein component of the |
| | | | | large 60S ribosomal subunit; human RPLP0 shows increased expression in human |
| | | | | hepatocellular and colon carcinomas |
| | | 580899\|Arbp | 0.0 | [Mus musculus] [Structural protein; RNA-binding protein; Ribosomal subunit] |
| | | | | [Cytoplasmic] Ribosomal protein P0, acidic phosphoprotein component of the |
| | | | | large 60S ribosomal subunit; human RPLP0 shows increased expression in human |
| | | | | hepatocellular and colon carcinomas |
| | | 243695\|F25H2.10 | 4.8E−53 | [Caenorhabditis elegans] [Complex assembly protein] [Cytoplasmic] Ortholog of |
| | | | | S. cerevisiae ribosomal protein Rpp0p and member of the acidic ribosomal protein |
| | | | | family |
| | | 370906\|rpp0 | 3.7E−47 | [Schizosaccharomyces pombe] [Ribosomal subunit] 60S acidic ribosomal protein |
| | | | | P0 |
| 21 | 7504128CD1 | g186800 | 2.3E−71 | [Homo sapiens] ribosomal protein L12 |
| | | | | Chu, W. et al. (1993) Nucleic Acids Res. 21: 749-749 |
| | | | | The primary structure of human ribosomal protein L12 |
| | | 337686\|RPL12 | 2.0E−72 | [Homo sapiens] [Structural protein; RNA-binding protein; Ribosomal subunit] |
| | | | | [Cytoplasmic] Ribosomal protein L12, component of the large 60S ribosomal |
| | | | | subunit |
| | | 429164\|Rpl12 | 6.8E−72 | [Mus musculus] [Structural protein; RNA-binding protein; Ribosomal subunit] |
| | | | | [Cytoplasmic] Ribosomal protein L12, a component of the 60S ribosomal subunit |
| | | 247161\|rpl-12 | 1.6E−54 | [Caenorhabditis elegans] [RNA-binding protein] [Cytoplasmic] Member of the |
| | | | | ribosomal protein L12 protein family |
| | | 371019\|rpl12-1 | 1.4E−38 | [Schizosaccharomyces pombe] [Ribosomal subunit] 60S ribosomal protein L12A, |
| | | | | has high similarity to S. cerevisiae Rpl12ap and S. cerevisiae Rpl12bp |
| | | 370868\|rpl12-2 | 1.4E−38 | [Schizosaccharomyces pombe] [Ribosomal subunit] 60S ribosomal protein L12B, |
| | | | | has high similarity to S. cerevisiae Rpl12ap and S. cerevisiae Rpl12bp |
| 22 | 4529338CD1 | g7542351 | 1.9E−187 | [Homo sapiens] QUAKING isoform 6 |
| | | 658120\|QKI | 1.6E−188 | [Homo sapiens] [RNA-binding protein] Protein with very strong similarity to |
| | | | | murine qk, which is a putative RNA-binding protein that functions during |
| | | | | embryonic myelination; mutations in the murine gene have effects ranging from |
| | | | | embryonic death to quaking due to demyelination |
| | | | | Ebersole, T. A. et al. (1996) Nat Genet 12: 260-265 |
| | | | | The quaking gene product necessary in embryogenesis and myelination combines |
| | | | | features of RNA binding and signal transduction proteins. |
| | | 626514\|qk | 6.7E−160 | [Mus musculus] [RNA-binding protein] Putative RNA-binding protein that has a |
| | | | | role in myelination during embryogenesis; mutations ofthe corresponding gene |
| | | | | have effects ranging from embryonic death to a transient quaking phenotype |
| | | | | caused by demyelination |
| | | 250880\|T21G5.5 | 1.8E−65 | [Caenorhabditis elegans] Putative paralog of C. elegans GLD-1 which encodes an |
| | | | | RNA-binding protein required for transition from mitosis to meiosis during |
| | | | | spermatogenesis and oogenesis in hermaphrodites |
| | | 251086\|gld-1 | 5.0E−61 | [Caenorhabditis elegans] [Inhibitor or repressor; RNA-binding protein] |
| | | | | [Cytoplasmic] RNA binding protein, required for transition from mitosis to |
| | | | | meiosis during spermatogenesis and oogenesis in hermaphrodites |
| | | 364843\|T-STAR | 5.2E−34 | [Homo sapiens] [RNA-binding protein; Small molecule-binding protein] |
| | | | | [Nuclear] RNA-binding protein that has similarity to theSrc associated SAM68 |
| | | | | protein, interacts with the testis-specific RBM RNA-binding protein and is |
| | | | | expressed primarily in the testis |
| 23 | 7503460CD1 | g899298 | 2.8E−67 | [Homo sapiens] human splicing factor |
| | | | | Kramer, A. et al. (1995) RNA 1: 260-272 |
| | | | | Mammalian splicing factor SF3a120 represents a new member of the SURP |
| | | | | family of proteins and is homologous to the essential splicing factor PRP21p of |
| | | | | Saccharomyces cerevisiae |
| | | 742382\|SF3A1 | 2.4E−68 | [Homo sapiens] Splicing factor 3a subunit 1, component of histone deacetylase |
| | | | | complexes, may be involved in transcriptional repression |
| | | 251943\|prp-21 | 2.0E−23 | [Caenorhabditis elegans] [RNA-binding protein] [Nuclear] Putative U2 snRNP- |
| | | | | associated splicing factor, putative ortholog of human SAP114/SF3a120 and yeast |
| | | | | Prp21p, member of the SWAP protein family |
| | | 369888\|sap114 | 1.3E−21 | [Schizosaccharomyces pombe] Pre-mRNA splicing factor |
| | | 341234\|SFRS8 | 6.8E−15 | [Homo sapiens] [Spliceosomal subunit; RNA-binding protein] [Nuclear] Splicing |
| | | | | factor arginine serine rich 8, a memberof the SR protein family, regulates |
| | | | | alternative splicing by influencing the selection of alternative 5′ splice sites, |
| | | | | affects alternative splicing of fibronectin, CD45 (PTPRC), and its own mRNA |
| | | 639178\|orf6.4710 | 5.0E−10 | [Candida albicans] Protein containing two Surpmodules (SWAP domain) which |
| | | | | may mediate RNA binding, has low similarity to a region of human SF3A120 |
| | | | | protein, which is the large subunit (p120) of the SF3A splicing factor and involved |
| | | | | in activation of U2 snRNP |
| 24 | 5466630CD1 | g7290296 | 1.8E−239 | [Drosophila melanogaster] kz gene product |
| | | 276103\|C06E1.10 | 6.9E−206 | [Caenorhabditis elegans] [Helicase] Member of the RNAhelicase, DEAH-box |
| | | | | protein family |
| | | 1295\|ECM16 | 9.7E−161 | [Saccharomyces cerevisiae] [Hydrolase; helicase; RNA-binding protein] [Nuclear |
| | | | | nucleolus; Nuclear] Putative DEAH-box RNA helicase, directly implicated in |
| | | | | ribosome biogenesis |
| | | | | Lussier, M. et al. (1997) Genetics 147: 435-450 |
| | | | | Large scale identification of genes involved in cell surface biosynthesis and |
| | | | | architecture in Saccharomyces cerevisiae. |
| | | 657990\|SPAPB1 | 2.8E−155 | [Schizosaccharomyces pombe] [Nuclear nucleolus] Putative ATP-dependent RNA |
| | | A10.06c | | helicase |
| | | 644688\|orf6.7465 | 2.9E−119 | [Candida albicans] [RNA-binding protein] Protein with high similarity to S. cerevisiae |
| | | | | Ecm16p, which is a putative DEAH-box RNA helicase directly |
| | | | | implicated in ribosome biogenesis, contains a helicase conserved C-terminal |
| | | | | domain |
| | | 371297\|prp22 | 1.8E−91 | [Schizosaccharomyces pombe] Putative pre-mRNA splicing factor ATP- |
| | | | | dependent RNA helicase |
| 25 | 7503474CD1 | g7243749 | 2.7E−124 | [Homo sapiens] sir2-related protein type 6 |
| | | | | Frye, R.A. (2000) Biochem. Biophys. Res. Commun. 273: 793-798 |
| | | | | Phylogenetic classification of prokaryotic and eukaryotic Sir2-like proteins |
| | | 476521\|SIRT6 | 2.4E−125 | [Homo sapiens] Protein with low similarity to SIRT3 and SIRT4, which are |
| | | | | putative ADP-ribosyltransferases, and to members of the Sir2p family of |
| | | | | transcriptional regulatory proteins |
| | | 476523\|SIRT7 | 4.9E−29 | [Homo sapiens] Protein with low similarity to members of the Sir2p family of |
| | | | | transcriptional regulatory proteins |
| | | 724128\|1ici_A | 3.6E−11 | [Protein Data Bank] Transcriptional Regulatory Protein, Sir2 Fam |
| | | 373770\|SPCC132.02 | 3.8E−10 | [Schizosaccharomyces pombe] [Transferase] Protein with high similarity to |
| | | | | human SIRT2, which is a putative NAD-dependent deacetylase and ADP- |
| | | | | ribosyltransferase, member of the Sir2 family, which are silent information |
| | | | | regulators |
| | | 642492\|orf6.6367 | 2.4E−09 | [Candida albicans] Member of the Sir2 family of putative NAD-dependent |
| | | | | histone deacetylases, which are involved in aging and chromatin structure and |
| | | | | some of which may have NAD-dependent mono-ADP-ribosyltransferase activity |
| | | | | has moderate similarity to a region of S. cerevisiae Sir2p |
| 26 | 7503498CD1 | g3249541 | 8.3E−152 | [Homo sapiens] ribonuclease P protein subunit p40 |
| | | | | Jarrous, N. (1998) RNA 4: 407-417 |
| | | | | Autoantigenic properties of some protein subunits of catalytically active |
| | | | | complexes of human ribonuclease P |
| | | 364641\|RPP40 | 7.3E−153 | [Homo sapiens] [Hydrolase; Nuclease (endo, exo, ribo, deoxyribo)] [Nuclear] |
| | | | | Subunit p40 of ribonuclease P ribonucleoprotein, which processes 5′ ends of |
| | | | | precursor tRNAs, does not react with Th sera from patients with systemic sclerosis |
| 27 | 7504119CD1 | g21039484 | 1.0E−132 | [fl][Mus musculus] transcription factor b1 |
| | | g13423097 | 9.7E−41 | [Caulobacter crescentus] dimethyladenosine transferase |
| | | 475717\|LOC51106 | 5.2E−150 | [Homo sapiens] [RNA-binding protein] Member of the ribosomal RNA adenine |
| | | | | dimethylase family |
| | | 249581\|T03F1.7 | 1.7E−60 | [Caenorhabditis elegans] [Transferase] [Nuclear ucleolus; Nuclear] Protein with |
| | | | | similarity to ribosomal RNA adenine dimethylases, has weak similarity to S. cerevisiae |
| | | | | dimethyladenosine transferase Dim1p |
| | | 373375\|SPBC336.02 | 7.0E−12 | [Schizosaccharomyces pombe] [Transferase] Dimethylase |
| 28 | 71532805CD1 | g307388 | 1.7E−74 | [Homo sapiens] ribosomal protein L7 |
| | | | | Seshadri, T. et al. (1993) J. Biol. Chem. 268: 18474-18480 |
| | | | | Identification of a transcript that is down-regulated in senescent human |
| | | | | fibroblasts: Cloning, sequence analysis and regulation of the human L7 ribosmal |
| | | | | protein gene |
| | | 337748\|RPL7 | 1.1E−74 | [Homo sapiens] [Structural protein; Ribosomal subunit; RNA-binding protein] |
| | | | | [Cytoplasmic] Ribosomal protein L7, component of the large 60S ribosomal |
| | | | | subunit; expression is reduced in senescent cells |
| | | 586417\|Rpl7 | 1.7E−74 | [Mus musculus] [Structural protein; Ribosomal subunit; RNA-binding protein] |
| | | | | [Cytoplasmic] Ribosomal protein L7, component of the large 60S ribosomal |
| | | | | subunit |
| | | 246052\|F53G12.10 | 1.1E−72 | [Caenorhabditis elegans] [RNA-binding protein] [Cytoplasmic] Member of the |
| | | | | ribosomal protein L7 protein family |
| | | 370375\|rpl7-2 | 6.9E−71 | [Schizosaccharomyces pombe] [Ribosomal subunit] 60S ribosomal protein |
| | | | | L7B/L7-C |
| | | 376060\|rpl7-1 | 4.9E−70 | [Schizosaccharomyces pombe] [Ribosomal subunit] 60S ribosomal protein L7A |
| 29 | 5502992CD1 | g7230509 | 0.0 | [Drosophila melanogaster] KISMET-L long isoform |
| | | | | Therrien, M. et al. (2000) Genetics 156: 1231-1242 |
| | | | | A genetic screen for modifiers of a kinase suppressor of ras-dependent rough eye |
| | | | | phenotype in Drosophila |
| | | 619200\|KIAA1564 | 0.0 | [Homo sapiens] Protein of unknown function, has a region of moderate similarity |
| | | | | to a region of human ZFH, which is a zinc finger helicase and a member of the |
| | | | | DNA helicase superfamily II |
| | | 249667\|T04D1.4 | 1.9E−296 | [Caenorhabditis elegans] [Helicase] [Nuclear] Member of the DNA helicase |
| | | | | protein family |
| | | 691900\|FLJ12178 | 5.5E−149 | [Homo sapiens] Protein of unknown function, has moderate similarity to a region |
| | | | | of human SMARCA2, which is a transcription cofactor that cooperates with |
| | | | | glucocorticoid receptor to activate transcription and is excluded from condensed |
| | | | | chromosomes |
| | | 247007\|H06O01.2 | 7.9E−145 | [Caenorhabditis elegans] [Helicase] [Nuclear] Putative chromodomain helicase |
| | | | | DNA binding protein |
| 30 | 7503828CD1 | g1549241 | 0.0 | [Homo sapiens] SWI/SNF complex 170 KDa subunit |
| | | | | Wang. W. et al. (1996) Genes Dev. 10: 2117-2130 |
| | | | | Diversity and specialization of mammalian SWI/SNF complexes. |
| | | 319202\|Smarcc1 | 0.0 | [Mus musculus] [Transcription factor] [Nuclear] SWI-SNF related matrix |
| | | | | associated actin dependent regulator of chromatin subfamilyc member 1, |
| | | | | chromatin binding protein implicated in regulation of transcription by remodeling |
| | | | | chromatin, may play role in T cell development and regulation of apoptosis |
| | | 338138\|SMARCC2 | 0.0 | [Homo sapiens] [Transcription factor] [Nuclear] Member 2 of subfamily c of |
| | | | | SWI/SNF related matrix associated actin dependent regulators of chromatin, part |
| | | | | of a complex involved in fetal to adult globin gene switching and part of a co- |
| | | | | repressor complex |
| | | 338136\|SMARCC1 | 0.0 | [Homo sapiens] [Transcription factor] [Nuclear] SWI-SNF related matrix |
| | | | | associated actin dependent regulator of chromatin subfamilyc member 1, a |
| | | | | putative trancription co-activator which is implicated in regulation of transcription |
| | | | | by remodeling nucleosomes and chromatin |
| | | 441839\|psa-1 | 5.0E−135 | [Caenorhabditis elegans] [DNA-binding protein] [Nuclear] Putative component of |
| | | | | a SWI/SNF chromatin remodeling complex, active in the control of mitosis |
| | | 372067\|SPAC23 | 2.2E−77 | [Schizosaccharomyces pombe] Protein with moderate similarity to S. cerevisiae |
| | | H3.10 | | Rsc8p |
| 31 | 2647325CD1 | g55471 | 1.8E−37 | [Mus musculus] Zfp-29 |
| | | | | Denny, P. and Ashworth, A. (1991) Gene 106: 221-227 |
| | | | | A zinc finger protein-encoding gene expressed in the post-meiotic phase of |
| | | | | spermatogenesis. |
| | | 322628\|Zfp29 | 1.6E−38 | [Mus musculus] [Transcription factor; DNA-binding protein] Zinc-finger protein |
| | | | | that may regulate post-meiotic germ cell gene expression, expressed specifically in |
| | | | | post-meiotic round spermatids |
| | | 339004\|ZNF84 | 1.0E−37 | [Homo sapiens] [Inhibitor or repressor; DNA-binding protein; Transcription |
| | | | | factor] [Nuclear] Protein containing a KRAB (kruppel-associated box) domain |
| | | | | which may mediate transcriptional repression and several C2H2 type zinc finger |
| | | | | domains, which bind nucleic acids |
| | | 338982\|ZNF205 | 1.2E−36 | [Homo sapiens] [Inhibitor or repressor; Transcription factor] Protein containing |
| | | | | C2H2 type zinc finger domains, which bind nucleic acids, and a KRAB (kruppel- |
| | | | | associated box) domain, which may mediate transcriptional repression |
| | | 338968\|ZNF157 | 1.2E−36 | [Homo sapiens] [Inhibitor or repressor; Transcription factor] Zinc finger protein |
| | | | | 157, a zinc-finger protein that contains two Kruppel-associated box (KRAB-A and |
| | | | | KRAB-B) transcription repression domains |
| | | 319698\|Zfp46 | 1.9E−36 | [Mus musculus] Zinc finger protein 46, contains an acidic domain followed by |
| | | | | C2H2 zinc finger domains in the N-terminal region, may bind to nucleic acids |
| 32 | 7495416CD1 | g488551 | 1.5E−77 | [Homo sapiens] zinc finger protein ZNF132 |
| | | | | Tommerup, N. and Vissing, H. (1995) Genomics 27: 259-264 |
| | | | | Isolation and fine mapping of 16 novel human zinc finger-encoding cDNAs |
| | | | | identify putative candidate genes for developmental and malignant disorders. |
| | | 476113\|LOC51333 | 3.2E−160 | [Homo sapiens] [DNA-binding protein] Protein containing aC2H2 type zinc |
| | | | | finger domain, which bind nucleic acids |
| | | 423343\|KIAA0326 | 9.5E−81 | [Homo sapiens] [DNA-binding protein] Protein containing nineteen C2H2 type |
| | | | | zinc finger domains, which bind nucleic acids |
| | | 338948\|ZNF132 | 1.3E−78 | [Homo sapiens] [Transcription factor] Zinc finger protein 132, a member of the |
| | | | | Kruppel zinc-finger protein family, contains tandemly repeated C2H2 zinc finger |
| | | | | domains |
| | | 324156\|Mm.10509 | 3.4E−78 | [Mus musculus] [DNA-binding protein] [Nuclear] Protein containing a C2H2 type |
| | | | | zinc finger domain, which bind nucleic acids |
| | | 338954\|ZNF135 | 4.3E−78 | [Homo sapiens] Member of the Kruppel family of zinc-finger proteins |
| 33 | 8096177CD1 | g7243633 | 5.7E−131 | [Homo sapiens] RB-associated KRAB repressor |
| | | | | Skapek, S. X. et al. (2000) J. Biol. Chem. 275: 7212-7223 |
| | | | | Cloning and characterization of a novel Kruppel-associated box family |
| | | | | transcriptional repressor that interacts with the retinoblastoma gene product, RB |
| | | 610561\|LOC57209 | 2.5E−201 | [Homo sapiens] [DNA-binding protein] Protein containing seven C2H2 type zinc |
| | | | | finger domains, which bind nucleic acids, has high similarity to a region of human |
| | | | | ZNF33A, which is a zinc finger protein |
| | | 424090\|KIAA0972 | 2.5E−137 | [Homo sapiens] [Inhibitor or repressor; Transcription factor] Protein containing a |
| | | | | KRAB (kruppel-associated box) domain which may mediate protein-protein |
| | | | | intereactions, contains C2H2 type zinc finger domains, which bind nucleic acids, |
| | | | | has moderate similarity to transcriptional repressors |
| | | 598470\|FLJ10469 | 1.0E−133 | [Homo sapiens] Inhibitor or repressor; Transcription factor; DNA-binding |
| | | | | protein] [Nuclear] Protein containing a KRAB (kruppel-associated box) domain |
| | | | | which may mediate transcriptional repression, and fourteen C2H2 type zinc finger |
| | | | | domains, which bind nucleic acids |
| | | 437244\|RBAK | 5.0E−132 | [Homo sapiens] Inhibitor or repressor; DNA-binding protein; Transcription |
| | | | | factor] [Nuclear] RB-associated KRAB protein, a member of the Kruppel- |
| | | | | associated box family of transcriptional repressors, interacts with the |
| | | | | retinoblastoma protein RB1 and may repress E2F-dependent genes |
| | | 339004\|ZNF84 | 9.6E−129 | [Homo sapiens] [Inhibitor or repressor; DNA-binding protein; Transcription |
| | | | | factor] [Nuclear] Protein containing a KRAB (kruppel-associated box) domain |
| | | | | which may mediate transcriptional repression and several C2H2 type zinc finger |
| | | | | domains, which bind nucleic acids |
| 34 | 666763CD1 | g12232096 | 4.2E−18 | [Caenorhabditis elegans] replication licensing factor MCM2/3/5-type protein |
| | | 253521\|ZK632.1 | 3.7E−19 | [Caenorhabditis elegans] [DNA-binding protein] [Nuclear] Member of the MCM |
| | | | | initiator complex (DNA replication) protein family |
| | | 1280\|CDC54 | 8.8E−17 | [Saccharomyces cerevisiae] [Hydrolase; DNA-binding protein; ATPase] [Nuclear] |
| | | | | Protein involved in DNA synthesis initiation, member of the MCM family of |
| | | | | DNA-dependent ATPases required for initiation of DNA replication |
| | | 637674\|orf6.3958 | 1.1E−16 | [Candida albicans] [Hydrolase; DNA-binding protein; ATPase]Protein with high |
| | | | | similarity to S. cerevisiae Cdc54p, which is involved in DNA synthesis initiation, |
| | | | | member of the MCM family of DNA-dependent ATPases, which may act as |
| | | | | replicative DNA helicases |
| 35 | 7504091CD1 | g13097225 | 1.1E−175 | [Homo sapiens] mitochondrial ribosomal protein L3 |
| | | 428458\|MRPL3 | 9.4E−177 | [Homo sapiens] [Structural protein; RNA-binding protein; Ribosomal subunit] |
| | | | | [Nuclear; Nuclear nucleolus; Cytoplasmic] Ribosomal protein L3, component of |
| | | | | the large 60S ribosomal subunit, may be involved in binding of the mRNA to the |
| | | | | ribosome |
| | | 713842\|C26E6.6 | 5.8E−39 | [Caenorhabditis elegans] Protein with weak similarity to ribosomal protein L3 |
| | | 7135\|MRPL9 | 7.0E−33 | [Saccharomyces cerevisiae] [RNA-binding protein; Ribosomal subunit] |
| | | | | [Mitochondrial] Mitochondrial ribosomal protein of the large subunit (YmL9; E. coli |
| | | | | L3; human MRL3) |
| | | 647232\|orf6.8737 | 4.2E−31 | [Candida albicans] [RNA-binding protein; Ribosomal subunit][Cytoplasmic] |
| | | | | Protein with high similarity to S. cerevisiae Mrp19p, which is a mitochondrial |
| | | | | ribosomal protein of the large subunit, protein of the large 60S ribosomal subunit |
| | | 616118\|SPAC644.17c | 4.1E−29 | [Schizosaccharomyces pombe] Mitochondrial ribosomal protein L9 |
| 36 | 7503568CD1 | g13172240 | 1.2E−161 | [Mus musculus] alpha-CP2; hnRNP-E2 |
| | | | | Makeyev, AV, Liebhaber, SA. Genomics (2000) Genomics 67: 301-316 |
| | | | | Identification of two novel mammalian genes establishes a subfamily of KH- |
| | | | | domain RNA-binding proteins. |
| | | 743096\|PCBP2 | 4.1E−161 | [Homo sapiens] [RNA-binding protein] Protein containing KHRNA-binding |
| | | | | domains, a major poly(rC)-binding protein together withPCBP1 and HNRPK |
| | | 343616\|PCBP1 | 1.7E−138 | [Homo sapiens] [RNA-binding protein] Poly(rC)-binding protein 1, contains KH |
| | | | | RNA-binding domains, binds poly(rC) RNA, acts as a translational repressor and |
| | | | | plays a role in mRNA stability |
| | | 430118\|Pcbp1 | 1.7E−138 | [Mus musculus] [RNA-binding protein] Poly(rC)-binding protein 1, contains KH |
| | | | | RNA-binding domains, binds poly(rC) RNA and may play a role in mRNA |
| | | | | stability |
| | | 613185\|PCBP3 | 4.3E−129 | [Homo sapiens] [RNA-binding protein] Poly(rC)-binding protein 3, a member of a |
| | | | | family of KH-domain containing RNA-binding proteins |
| | | 618966\|Pcbp3 | 3.8E−128 | [Mus musculus] [Nuclear] Protein with high similarity to murine Pcbp2 (secreted |
| | | | | phosphoprotein), which contains KHRNA-binding domains and binds |
| | | | | preferentially to oligo dC |
| 37 | 7504101CD1 | g882258 | 0.0 | [Homo sapiens] chromatin assembly factor-I p150 subunit |
| | | | | Kaufman, P.D. et al. (1995) Cell 81: 1105-1114 |
| | | | | The p150 and p60 subunits of chromatin assembly factor I: a molecular link |
| | | | | between newly synthesized histones and DNA replication. |
| | | 341970\|CHAF1A | 0.0 | [Homo sapiens] [Complex assembly protein; Chaperones; DNA-binding protein] |
| | | | | [Nuclear] Chromatin assembly factor 1 subunit A, chromatin assembly factor 1 |
| | | | | subunit that mediates deposition of newly synthesized histones H3 and acetylated |
| | | | | H4 onto replicated DNA, may mediate a chromatin assembly response to DNA |
| | | | | damage by interacting with PCNA |
| | | 433052\|Chaf1a | 2.0E−224 | [Mus musculus] [Complex assembly protein; DNA-binding protein] [Nuclear] |
| | | | | Chromatin assembly factor 1 subunit A, chromatin assembly factor 1 subunit that |
| | | | | interacts with HP1 proteins, may modulate chromatin and heterochromatin |
| | | | | dynamics; human CHAF1A may mediate a chromatin assembly response to DNA |
| | | | | damage by interacting with PCNA |
| | | 441141\|T06D10.2 | 9.5E−35 | [Caenorhabditis elegans] Protein with moderate similarity to C. elegans F36H12.3 |
| | | 631024\|orf6.633 | 3.2E−26 | [Candida albicans] Protein of unknown function, has low similarity to S. cerevisiae |
| | | | | Rlf2p, which is a subunit of the chromatin assembly complex involved |
| | | | | in nucleosome assembly linked with DNA replication |
| | | 639148\|orf6.4695 | 3.2E−26 | [Candida albicans] Protein of unknown function, has low similarity to S. cerevisiae |
| | | | | Rlf2p, which is subunit 1 of the chromatin assembly complex involved |
| | | | | in nucleosome assembly linked with DNA replication |
| 38 | 6946680CD1 | g13560888 | 8.6E−160 | [Homo sapiens] EZFIT-related protein 1 |
| | | 308339\|ZNF184 | 1.7E−154 | [Homo sapiens] Kruppel-like zinc-finger protein, maximally expressed in testis, |
| | | | | moderately in other tissues |
| | | 339006\|ZNF85 | 1.6E−142 | [Homo sapiens] [Inhibitor or repressor; Transcription factor; DNA-binding |
| | | | | protein] [Nuclear] Zinc finger protein 85, member of the ZNF91 family of |
| | | | | Kruppel-associated box (KRAB) zinc finger proteins, functions as a |
| | | | | transcriptional co-repressor |
| | | 432896\|ZNF208 | 2.7E−140 | [Homo sapiens] Zinc finger protein 208, a ubiquitously expressed Kruppel- |
| | | | | associated box (KRAB) zinc finger protein |
| | | 339004\|ZNF84 | 4.4E−140 | [Homo sapiens] [Inhibitor or repressor; DNA-binding protein; Transcription |
| | | | | factor] [Nuclear] Protein containing a KRAB (kruppel-associated box) domain |
| | | | | which may mediate transcriptional repression and several C2H2 type zinc finger |
| | | | | domains, which bind nucleic acids |
| | | 475040\|HSPC059 | 3.6E−138 | [Homo sapiens] [Inhibitor or repressor; Transcription factor; DNA-binding |
| | | | | protein] Protein containing sixteen C2H2 type zinc finger domains, which bind |
| | | | | nucleic acids, contains a KRAB (kruppel-associated box) domain which may |
| | | | | mediate transcriptional repression |
| 39 | 7001142CD1 | g13560888 | 7.8E−166 | [Homo sapiens] EZFIT-related protein 1 |
| | | 308339\|ZNF184 | 2.2E−145 | [Homo sapiens] Kruppel-like zinc-finger protein, maximally expressed in testis, |
| | | | | moderately in other tissues |
| | | 619192\|KIAA1559 | 2.4E−141 | [Homo sapiens] Protein with strong similarity to murine Zfp30, which is a zinc- |
| | | | | finger protein containing a Kruppel-associated box (KRAB) transcriptional |
| | | | | repression domain |
| | | 424068\|KIAA0961 | 1.5E−139 | [Homo sapiens] Protein with strong similarity to murine Zfp30, which is a zinc- |
| | | | | finger protein containing a Kruppel-associated box (KRAB) transcriptional |
| | | | | repression domain |
| | | 475040\|HSPC059 | 6.7E−135 | [Homo sapiens] [Inhibitor or repressor; Transcription factor; DNA-binding |
| | | | | protein] Protein containing sixteen C2H2 type zinc finger domains, which bind |
| | | | | nucleic acids, contains a KRAB (kruppel-associated box) domain which may |
| | | | | mediate transcriptional repression |
| | | 339004\|ZNF84 | 8.5E−135 | [Homo sapiens] [Inhibitor or repressor; DNA-binding protein; Transcription |
| | | | | factor] [Nuclear] Protein containing a KRAB (kruppel-associated box) domain |
| | | | | which may mediate transcriptional repression and several C2H2 type zinc finger |
| | | | | domains, which bind nucleic acids |
| 40 | 71158380CD1 | g4519270 | 2.8E−264 | [Homo sapiens] Kruppel-type zinc finger protein |
| | | | | Katoh, O. et al. (1998) Biochem. Biophys. Res. Commun. 249: 595-600 |
| | | | | ZK1, a novel Kruppel-type zinc finger gene, is induced following exposure to |
| | | | | ionizing radiation and enhances apoptotic cell death on hematopoietic cells |
| | | 700794\|FLJ14356 | 1.6E−284 | [Homo sapiens] Protein with high similarity to human ZNF136, which is a C2H2 |
| | | | | zinc-finger protein that represses transcription when fused to the heterologous |
| | | | | KRAB B subdomain of human ZNF10 |
| | | 342918\|ZK1 | 2.5E−265 | [Homo sapiens] Kruppel-type zinc finger protein, has an A box of Kruppel- |
| | | | | associated box (KRAB) domain and fifteen zinc finger motifs, possibly functions |
| | | | | in radiation-induced apoptosis, expression is induced by exposure to ionizing |
| | | | | radiation |
| | | 476341\|GIOT-2 | 2.6E−254 | [Homo sapiens] [Inhibitor or repressor; Transcription factor; DNA-binding |
| | | | | protein] Protein containing fifteen C2H2 type zinc finger domains, which bind |
| | | | | nucleic acids, also contains a KRAB (kruppel-associated box) domain which may |
| | | | | mediate transcriptional repression |
| | | 594469\|HSZFP36 | 3.6E−248 | [Homo sapiens] [Inhibitor or repressor; Transcription factor; DNA-binding |
| | | | | protein] Protein containing fourteen C2H2 type zinc finger domains, which bind |
| | | | | nucleic acids, also contains a KRAB (kruppel-associated box) domain which may |
| | | | | mediate transcriptional repression |
| | | 476345\|LOC51712 | 1.5E−203 | [Homo sapiens] [Inhibitor or repressor; Transcription factor; DNA-binding |
| | | | | protein] Protein containing eighteen C2H2 type zinc finger domains, which bind |
| | | | | nucleic acids, also contains a KRAB (kruppel-associated box) domain which may |
| | | | | mediate transcriptional repression |
| 41 | 7503861CD1 | g14764499 | 1.1E−171 | [Homo sapiens] zinc finger protein |
| | | 346716\|KIAA0211 | 0.0 | [Homo sapiens] Protein containing C2H2 type zinc finger domains, which bind |
| | | | | nucleic acids |
| | | 598616\|FLJ10697 | 1.1E−111 | [Homo sapiens] [DNA-binding protein] Protein with a low similarity to KRAB |
| | | | | zinc finger proteins |
| | | 423343\|KIAA0326 | 4.7E−21 | [Homo sapiens] [DNA-binding protein] Protein containing nineteen C2H2 type |
| | | | | zinc finger domains, which bind nucleic acids |
| | | 342394\|ZNF256 | 3.4E−20 | [Homo sapiens] [Inhibitor or repressor; Transcription factor; DNA-binding |
| | | | | protein] Zinc finger protein 256, a putative transcriptional repressor that may play |
| | | | | a role in hemopoiesis, member of the Kruppel-like zinc-finger family |
| | | | | Han, Z. G. et al. (1999) J. Biol. Chem. 274: 35741-35748 |
| | | | | Molecular cloning of six novel Kruppel-like zinc finger genes from hematopoietic |
| | | | | cells and identification of a novel transregulatory domain KRNB. |
| | | 338994\|ZNF43 | 1.7E−19 | [Homo sapiens] Zinc finger protein 43, contains C2H2 zinc finger motifs, |
| | | | | expressed mainly in B and T cells |
| 42 | 7758395CD1 | g15553139 | 9.6E−104 | [Homo sapiens] (AF297872) zinc finger transcription factor TReP-132 |
| | | | | Gizard, F. (2001) J. Biol. Chem. 276: 33881-33892 |
| | | | | A novel zinc finger protein TReP-132 interacts with CBP/p300 to regulate human |
| | | | | CYPI1A1 [steroid synthesis] gene expression |
| | | 594951\|HSA277276 | 4.2E−99 | [Homo sapiens] [DNA-binding protein] [Nuclear] Protein containing a Myb-like |
| | | | | DNA-binding domain and two C2H2 type zinc finger domain, which bind nucleic |
| | | | | acids |
| | | 246070\|F53H10.2 | 3.3E−22 | [Caenorhabditis elegans] Protein with weak similarity to C. elegans D1014.9 gene |
| | | | | product |
| | | 614095\|Brd4 | 3.6E−12 | [Mus musculus] Mitotic chromosome-associated protein, a member of the |
| | | | | bromodomain superfamily BET subgroup, associates with mitotic chromosomes |
| | | | | and functions in chromosomal dynamics during G(2)/M transition |
| | | 645094\|orf6.7668 | 2.8E−11 | [Candida albicans] Protein containing a pleckstrin homology (PH) domain, which |
| | | | | mediate protein-protein and protein-lipid interactions, has a region of low |
| | | | | similarity to a region of S. pombe Php5p, which is a subunit of CCAAT-binding |
| | | | | factor |
| 43 | 71039312CD1 | g7296687 | 1.4E−65 | [Drosophila melanogaster] cas gene product |
| | | | | Adams, M. D. et al. (2000) The genome sequence of Drosophila melanogaster. |
| | | | | Science 287: 2185-2195. |
| | | 599292\|FLJ20321 | 0.0 | [Homo sapiens] [DNA-binding protein] [Nuclear] Protein containing five C2H2 |
| | | | | type zinc finger domains, which bind nucleic acids. |
| 44 | 7291318CD1 | g5640019 | 4.0E−52 | [Mus musculus] zinc finger protein ZFP235 |
| | | 658380\|ZFP93 | 1.3E−54 | [Homo sapiens] Member of the XRCC1-linked KRAB zinc-finger protein family, |
| | | | | has similarity tomurine Zfp93. |
| | | | | Shannon, M. et al. (1996) Comparative analysis of a conserved zinc finger gene |
| | | | | cluster on human chromosome 19q and mouse chromosome 7. Genomics 33: 112-20. |
| | | 570912\|ZNF226 | 4.0E−54 | [Homo sapiens] [Inhibitor or repressor; Transcription factor; DNA-binding |
| | | | | protein] Protein containing eighteen C2H2 type zinc finger domains, which bind |
| | | | | nucleic acids, a KRAB (kruppel-associated box) domain which may mediate |
| | | | | transcriptional repression. |
| | | 434624\|ZNF234 | 2.1E−52 | [Homo sapiens] Member of the Kruppel-related zinc finger protein family. |
| | | | | Abrink, M. et al. (2001) Conserved interaction between distinct Kruppel- |
| | | | | associated box domains and the transcriptional intermediary factor 1 beta. Proc. |
| | | | | Natl. Acad. Sci. U.S.A. 98: 1422-1426. |
| 45 | 2638619CD1 | g2529737 | 5.7E−76 | [Xenopus laevis] ER1 |
| | | | | Paterno, G.D. et al. (1997) cDNA cloning of a novel, developmentally-regulated |
| | | | | immediate early gene activated by fibroblast growth factor and encoding a nuclear |
| | | | | protein. J. Biol. Chem. 272: 25591-25595. |
| | | 556774\|KIAA1193 | 1.1E−296 | [Homo sapiens] [DNA-binding protein] Protein containing a Myb DNA-binding |
| | | | | domain, and an uncharacterized ELM2 domain, which are found in C. elegans egl- |
| | | | | 27 and human and rat MTA1. |
| 46 | 2810014CD1 | g6601438 | 9.6E−35 | [Homo sapiens] AF5q31 protein |
| | | | | Taki, T. et al. (1999) AF5q31, a newly identified AF4-related gene, is fused to |
| | | | | MLL in |
| | | | | infant acute lymphoblastic leukemia with ins(5; 11)(q31; q13q23) |
| | | | | Proc. Natl. Acad. Sci. U.S.A. 96: 14535-14540. |
| | | 436208\|AF5Q31 | 8.4E−36 | [Homo sapiens] [DNA-binding protein; Transcription factor] ALL1 fused gene |
| | | | | from 5q31, a putative transcription factor; corresponding gene is fused to MLL in |
| | | | | cases of acute lymphoblastic leukemia as a result of genetic rearrangements. |
| | | | | Taki, T. et al. (1999) AF5q31, a newly identified AF4-related gene, is fused to |
| | | | | MLL in infant acute lymphoblastic leukemia with ins (5; 11) (q31; q13q23). Proc. |
| | | | | Natl. Acad. Sci. U.S.A. 96: 14535-14540. |
| | | | | Hillman, M. A. and Gecz, J. (20001) Fragile XE-associated familial mental |
| | | | | retardation protein 2 (FMR2) acts as a potent transcription activator. J. Hum |
| | | | | Genet. 46: 251-259. |
| 47 | 3457155CD1 | g5811583 | 0.0 | [Rattus norvegicus] TIP120-family protein TIP120B |
| | | | | Aoki, T. et al. (1999) TIP120B: a novel TIP120-family protein that is expressed |
| | | | | specifically in muscle tissues. Biochem. Biophys. Res. Commun. 261: 911-916. |
| | | 423639\|KIAA0667 | 0.0 | [Homo sapiens] TBP-interacting protein 120B. |
| | | 600204\|TIP120 | 0.0 | [Homo sapiens] [Nuclear] mRNA for KIAA0829 gene, isolated from human brain |
| | | | | cDNA library. |
| | | 332994\|Rn.32934 | 0.0 | [Rattus norvegicus] [Transcription factor] [Nuclear] TBP-interacting protein that |
| | | | | may play a role in transcriptional regulation. |
| 48 | 7435171CD1 | g3395529 | 8.7E−183 | [Mus musculus] homeodomain protein |
| | | 583221\|Hmx3 | 6.6E−146 | [Mus musculus] [Transcription factor; DNA-binding protein] H6 homeobox 3, a |
| | | | | DNA binding protein that is required for the formation of the inner ear vestibular |
| | | | | system, may function in neuronal cell specification; deficiency causes |
| | | | | reproductive defects in females and balance defects. |
| | | | | Wang, W. et al. (1998) Inner ear and maternal reproductive defects in mice |
| | | | | lacking the Hmx3 homeobox gene. Dev. Suppl. 125: 621-634. |
| 49 | 7499936CD1 | g9931482 | 2.2E−81 | [Cloning vector pFB-ERV] retinoic acid receptor RXR |
| | | 321064\|Rxra | 3.5E−85 | [Mus musculus] [Activator; Transcription factor; DNA-binding protein; Receptor |
| | | | | (signalling)] [Nuclear] Retinoid X receptor alpha, a high affinity receptor for 9-cis |
| | | | | retinoic acid, controls multiple metabolic pathways by interacting with a variety of |
| | | | | nuclear receptors and regulating transcriptional activity. |
| | | | | Mangelsdorf, D. J. et al. (1990) Nuclear receptor that identifies a novel retinoic |
| | | | | acid response pathway. Nature 345: 224-229. |
| | | 717364\|1fm6_A | 2.0E−82 | [Protein Data Bank] Retinoic Acid Receptor Rxr-Alpha. |
| | | | | Fournes, B. et at. (2001) The CEACAM1-L Ser503 residue is crucial for |
| | | | | inhibition of colon cancer cell tumorigenicity. Oncogene 20: 219-230. |
| 50 | 7504125CD1 | g531523 | 1.1E−71 | [Homo sapiens] Net |
| | | | | Giovane, A. et al. (1994) Net, a new ets transcription factor that is activated by |
| | | | | Ras Genes Dev. 8: 1502-1513. |
| | | 342016\|ELK3 | 9.7E−73 | [Homo sapiens] [DNA-binding protein; Transcription factor] [Nuclear] ETS- |
| | | | | domain protein (SRF accessory protein 2), a member of the ets family of |
| | | | | transcription factors that regulates transcription and serves as a target of Ras- |
| | | | | MAPK signal transduction pathways. |
| | | | | Price, M. A. (1995) Comparative analysis of the ternary complex factors Elk-1, |
| | | | | SAP-1 a and SAP-2 (ERP/NET). Embo Journal 14: 2589-2601. |
| 51 | 7505742CD1 | g516381 | 3.5E−266 | [Homo sapiens] transcription factor |
| | | | | Murphy, D.B. et al. (1994) Human brain factor 1, a new member of the fork head |
| | | | | gene family. Genomics 21: 551-557. |
| | | 342038\|FOXG1B | 3.0E−267 | [Homo sapiens] [DNA-binding protein; Transcription factor] Member of the HNF- |
| | | | | 3/fork head family of transcriptional regulators, expression is limited to the |
| | | | | neuronal cells in the telencephalon. |
| | | | | Pierrou, S. et al. (1994) Cloning and characterization of seven human forkhead |
| | | | | proteins: binding site specificity and DNA bending. Embo Journal 13: 5002-5012. |
| 52 | 7505757CD1 | g5811585 | 0.0 | [Rattus norvegicus] TIP120-family protein TIP120B, alternatiely spliced form |
| | | | | Aoki, T. et al. (1999) TIP120B: a novel TIP120-family protein that is expressed |
| | | | | specifically in muscle tissues. Biochem. Biophys. Res. Commun. 261: 911-916. |
| | | 423639\|KIAA0667 | 0.0 | [Homo sapiens] TBP-interacting protein 120B. |
| | | 600204\|TIP120 | 0.0 | [Homo sapiens] [Nuclear] mRNA for KIAA0829 gene, isolated from human brain |
| | | | | cDNA library. |
| | | | | Yogosawa, S. et al. (1999) Induced expression, localization, and chromosome |
| | | | | mapping of a gene for the TBP-interacting protein 120A. Biochem. Biophys. Res. |
| | | | | Commun. 266: 123-128. |
| 53 | 7504126CD1 | g3717978 | 1.4E−41 | [Mus musculus] 5S ribosomal protein |
| | | | | Vizirianakis, I.S. et al. (1999) Expression of ribosomal protein S5 cloned gene |
| | | | | during differentiation and apoptosis in murine erythroleukemia (MEL) cells. |
| | | | | Oncol. Res. 11: 409-419. |
| | | 709567\|RPS5 | 1.4E−43 | [Homo sapiens] [Structural protein; RNA-binding protein; Ribosomal subunit] |
| | | | | [Cytoplasmic] Ribosomal protein S5, a component of the 40S ribosomal subunit; |
| | | | | gene expression is altered in colorectal carcinoma cells. |
| | | | | Vizirianakis, I. S. et al. (1999) Expression of ribosomal protein S5 cloned gene |
| | | | | during differentiation and apoptosis in murine erythroleukemia (MEL) cells. |
| | | | | Oncol. Res. 11: 409-419. |
| 54 | 7504099CD1 | g871299 | 2.9E−173 | [Homo sapiens] Human pre-mRNA cleavage factor I 68 kDa subunit |
| | | | | Ruegsegger, U. et al. (1998) Human pre-mRNA cleavage factor Im is related to |
| | | | | spliceosomal SR proteins and can be reconstituted in vitro from recombinant |
| | | | | subunits. Mol. Cell 1: 243-253. |
| | | 428272\|CPSF6 | 2.5E−174 | [Homo sapiens] [RNA-binding protein] [Nuclear] Cleavage and polyadenylation |
| | | | | specific factor 6, a putative mRNA-binding protein that is the 68 kDa subunit of |
| | | | | the mRNA cleavage factor Im (CF Im) complex, plays a role in pre-mRNA 3′ end |
| | | | | processing. |
| | | | | de Vries, H. et al. (2000) Human pre-mRNA cleavage factor II(m) contains |
| | | | | homologs of yeast proteins and bridges two other cleavage factors Embo Journal |
| | | | | 19: 5895-5904. |
| 55 | 7505733CD1 | g2098734 | 1.6E−39 | [Homo sapiens] integrase |
| | | 476591\|HSU88895 | 1.2E−52 | [Homo sapiens] Putative protein encoded by human endogenous retrovirus H |
| | | | | (HERV-H). |
| | | | | Lindeskog, M. and Blomberg, J. (1997) Spliced human endogenous retroviral |
| | | | | HERV-H env transcripts in T-cell leukaemia cell lines and normal leukocytes: |
| | | | | alternative splicing pattern of HERV-H transcripts [published erratum appears in |
| | | | | J. Gen. Virol. 1998 Jan; 79 (Pt 1): 212] J. Gen. Virol., 2575-2585. |
| 56 | 7959829CD1 | g9652099 | 1.0E−69 | [Mus musculus] pseudouridine synthase 3 |
| | | | | Chen, J. and Patton, J. R. (2000) Pseudouridine synthase 3 from mouse modifies |
| | | | | the anticodon loop of tRNA. Biochemistry 39: 12723-12730. |
| | | 703953\|FKSG32 | 9.1E−85 | [Homo sapiens] Protein with moderate similarity to S. cerevisiae Deg1p, which is |
| | | | | a pseudouridine synthase that catalyzes the formation of pseudouridine-38 and -39 |
| | | | | in cytoplasmic and mitochondrial tRNAs. |
| 57 | 7502168CD1 | g52977 | 3.9E−212 | [Mus musculus] modifier 3 (M33) |
| | | | | Pearce, J. J. et al. (1992) The mouse has a Polycomb-like chromobox gene. |
| | | | | Development 114: 921-929. |
| | | 321346\|Cbx2 | 3.4E−213 | [Mus musculus] Homolog of Drosophila polycomb chromobox, which is |
| | | | | implicated in clonal inheritance of determined states through effects on chromatin |
| | | | | structure; mutation in the gene causes sex reversal. |
| | | | | Katoh-Fukui, Y. et al. (1998) Male-to-female sex reversal in M33 mutant mice. |
| | | | | Nature 393: 688-692. |
| 58 | 7503888CD1 | g10946128 | 0.0 | [Homo sapiens] SMARCA4 isoform 1 |
| | | | | Wong, A. K. C. et al. (2000) BRG1, a component of the SWI-SNF complex, is |
| | | | | mutated in multiple human tumor cell lines. Cancer Res. 60: 6171-6177. |
| | | 338130\|SMARC | 0.0 | [Homo sapiens] [Hydrolase; Activator; Helicase; Transcription factor; ATPase] |
| | | A4 | | [Nuclear] SWI-SNF related matrix associated actin-dependent regulator of |
| | | | | chromatin subfamily, a member 4, mediates transcriptional regulation by nuclear |
| | | | | receptors, RB1, Myc, KLF1 and BRCA1, involved in cell cycle control and T cell |
| | | | | receptor signaling. |
| | | | | Kadam, S. et al. (2000) Functional selectivity of recombinant mammalian |
| | | | | SWI/SNF subunits Genes And Development 14: 2441-2451. |

TABLE 3

| | | Amino | | Potential | | Analytical |
|---|---|---|---|---|---|---|
| SEQ | Incyte | Acid | Potential | Glycosyl- | | Methods |
| ID | Polypep- | Resi- | Phosphorylation | ation | | and |
| NO: | tide ID | dues | Sites | Sites | Signature Sequences, Domains and Motifs | Databases |
| 1 | 7503848CD1 | 1374 | S83 S87 S88 S120 | N109 N166 | PROTEIN TREACHER COLLINS SYNDROME | BLAST_{—} |
| | | | S153 S156 S171 | N572 N759 | TREACLE PUTATIVE NUCLEOLAR | PRODOM |
| | | | S198 S199 S205 | N1180 | TRAFFICKING PHOSPHOPROTEIN REPEAT | |
| | | | S206 S217 S264 | | PD017611: P1048-P1322 | |
| | | | S265 S270 S272 | | | |
| | | | S288 S304 S335 | | | |
| | | | S336 S340 S342 | | | |
| | | | S353 S369 S400 | | | |
| | | | S401 S405 S407 | | | |
| | | | S470 S471 S479 | | | |
| | | | S533 S576 S617 | | | |
| | | | S618 S622 S624 | | | |
| | | | S687 S688 | | | |
| | | | S692 S707 S724 | | TREACHER COLLINS SYNDROME PROTEIN | BLAST_{—} |
| | | | S793 S794 S798 | | TREACLE DISEASE MUTATION | PRODOM |
| | | | S800 S813 S829 | | POLYMORPHISM PUTATIVE NUCLEOLAR | |
| | | | S857 S858 S862 | | PD017236: Q563-R911 | |
| | | | S864 S920 S922 | | | |
| | | | S924 S965 S969 | | | |
| | | | S1027 S1039 S1041 | | | |
| | | | S1063 S1076 S1077 | | | |
| | | | S1143 S1160 S1223 | | | |
| | | | S1224 S1230 S1236 | | | |
| | | | S1247 S1324 S1331 | | | |
| | | | S1351 S1359 | | | |
| | | | T45 T98 T102 | | TREACHER COLLINS SYNDROME TREACLE | BLAST_{—} |
| | | | T129 T144 T173 | | PROTEIN DISEASE MUTATION | PRODOM |
| | | | T210 T609 T785 | | POLYMORPHISM PD038028: A411-P562 | |
| | | | T906 T916 T983 | | | |
| | | | T1007 T1067 | | | |
| | | | T1072 T1108 | | | |
| | | | T1219 T1244 | | | |
| | | | T1271 T1369 | | | |
| | | | | | PROTEIN TREACHER COLLINS SYNDROME | BLAST_{—} |
| | | | | | TREACLE PUTATIVE NUCLEOLAR | PRODOM |
| | | | | | TRAFFICKING PHOSPHOPROTEIN REPEAT | |
| | | | | | PD016387: P103-A250 | |
| | | | | | ACIDIC SERINE CLUSTER REPEAT DM04746 | BLAST_DOMO |
| | | | | | \|S57757\|1-646: E9-T629 | |
| | | | | | \|P41777\|1-386: K502-E828 | |
| | | | | | \|I38073\|1-377: M1-S369 | |
| | | | | | do NEUROFILAMENT; TRIPLET; | BLAST_DOMO |
| | | | | | DM04498\|P12036\|434-1019: T210-S798 | |
| | | | | | Atp_Gtp_A: A149-S156, A310-T317, A663-T670, | MOTIFS |
| | | | | | A835-T842 | |
| 2 | 2608080CD1 | 588 | S103 S112 S151 | N302 N358 | signal_cleavage: M1-T17 | SPSCAN |
| | | | S209 T19 T41 T69 | N379 N470 | | |
| | | | T173 T200 T293 | | | |
| | | | T334 T349 T405 | | | |
| | | | | | KRAB box: V9-K71 | HMMER_PFAM |
| | | | | | Zinc finger, C2H2 type: Y227-H249, F367-H389, | HMMER_PFAM |
| | | | | | L479-H501, F423-H445, Y395-H417, Y507-H529, | |
| | | | | | Y199-H221, F535-H557, F563-H585, Y283-H305, | |
| | | | | | Y451-H473, Y339-H361, Y255-H277, F311-H333 | |
| | | | | | Zinc finger, C2H2 type, domain proteins BL00028: | BLIMPS_{—} |
| | | | | | C257-H273 | BLOCKS |
| | | | | | C2H2-type zinc finger signature PR00048: P254- | BLIMPS_{—} |
| | | | | | R267, L550-G559 | PRINTS |
| | | | | | PROTEIN ZINC-FINGER METAL PD00066: H245- | BLIMPS_{—} |
| | | | | | C257 | PRODOM |
| | | | | | PROTEIN ZINC FINGER ZINC PD01066: F111-G49 | BLIMPS_{—} |
| | | | | | | PRODOM |
| | | | | | PROTEIN ZINC FINGER METAL BINDING DNA | BLAST_{—} |
| | | | | | BINDING ZINC FINGER PATERNALLY | PRODOM |
| | | | | | EXPRESSED ZN FINGER PW1 PD017719: G251- | |
| | | | | | H501; G223-V465; P310-H557 | |
| | | | | | HYPOTHETICAL ZINC FINGER PROTEIN | BLAST_{—} |
| | | | | | B03B8.4 IN CHROMOSOME III ZINC FINGER | PRODOM |
| | | | | | DNA BINDING METAL BINDING NUCLEAR | |
| | | | | | PD149420: R307-G475 | |
| | | | | | ZINC FINGER DNA BINDING PROTEIN METAL | BLAST_{—} |
| | | | | | BINDING NUCLEAR ZINC FINGER | PRODOM |
| | | | | | TRANSCRIPTION REGULATION REPEAT | |
| | | | | | PD000072: K393-C456 | |
| | | | | | ZINCFINGER METAL BINDING DNA BINDING | BLAST_{—} |
| | | | | | PROTEIN FINGER ZINC NUCLEAR REPEAT | PRODOM |
| | | | | | TRANSCRIPTION REGULATION PD001562: V9- | |
| | | | | | K71 | |
| | | | | | KRAB BOX DOMAIN DM00605 | BLAST_DOMO |
| | | | | | \|I48689\|11-85: V9-C79 | |
| | | | | | \|P51786\|24-86: V9-W68 | |
| | | | | | \|P52736\|1-72: V9-C79 | |
| | | | | | ZINC FINGER, C2H2 TYPE, DOMAIN | BLAST_DOMO |
| | | | | | DM00002\|Q05481\|789-829: R247-E287; R387-E427; | |
| | | | | | K414-E455 | |
| | | | | | Zinc_Finger_C2h2: C201-H221, C229-H249, C257- | MOTIFS |
| | | | | | H277, C285-H305, C313-H333, C341-H361, C369- | |
| | | | | | H389, C397-H417, C425-H445, C453-H473, C481- | |
| | | | | | H501, C509-H529, C537-H557, C565-H585 | |
| 3 | 7503402CD1 | 607 | S65 S69 S151 S276 | N272 N410 | signal_cleavage: M1-A39 | SPSCAN |
| | | | S481 S586 T35 T91 | N479 N573 | | |
| | | | T195 T279 T368 | | | |
| | | | T394 T443 | | | |
| | | | | | Zinc finger, C2H2 type: Y266-H290, Y236-H260, | HMMER_PFAM |
| | | | | | F206-H230, Y386-H409, Y326-H350, Y356-H380, | |
| | | | | | F296-H320 | |
| | | | | | Zinc finger, C2H2 type, domain proteins BL00028: | BLIMPS_{—} |
| | | | | | C388-H404 | BLOCKS |
| | | | | | PROTEIN ZINC-FINGER META PD00066: H256- | BLIMPS_{—} |
| | | | | | C268 | PRODOM |
| | | | | | SELENOCYSTEINE TRNA PROTEIN | BLAST_{—} |
| | | | | | TRANSCRIPTION ACTIVATING FACTOR DNA | PRODOM |
| | | | | | BINDING ZINC FINGER METAL BINDING ZINC | |
| | | | | | PD016532: D38-G172 | |
| | | | | | SELENOCYSTEINE TRNA TRANSCRIPTION | BLAST_{—} |
| | | | | | ACTIVATING FACTOR PROTEIN DNA BINDING | PRODOM |
| | | | | | ZINC FINGER METAL BINDING GENE | |
| | | | | | PD016467: H382-D449 | |
| | | | | | TRANSCRIPTION SELENOCYSTEINE TRNA | BLAST_{—} |
| | | | | | PROTEIN ACTIVATING FACTOR GENE ZINC | PRODOM |
| | | | | | FINGER REGULATION PD155356: V450-S518 | |
| | | | | | SELENOCYSTEINE TRNA PROTEIN | BLAST_{—} |
| | | | | | TRANSCRIPTION ACTIVATING FACTOR DNA | PRODOM |
| | | | | | BINDING ZINC FINGER METAL BINDING ZINC | |
| | | | | | PD034459: V519-G600 | |
| | | | | | do ACTIVATING; SELENOCYSTEINE; TRNA; | BLAST_DOMO |
| | | | | | DM04750 | |
| | | | | | \|P52747\|427-625: A408-D607 | |
| | | | | | \|S58681\|465-600: G489-D606 | |
| | | | | | ZINC FINGER, C2H2 TYPE, DOMAIN DM00002 | BLAST_DOMO |
| | | | | | \|P52747\|212-244: R193-H226 | |
| | | | | | \|P52747\|396-425: H377-T407 | |
| | | | | | Zinc_Finger_C2h2: C208-H230, C238-H260, C268- | MOTIFS |
| | | | | | H290, C298-H320, C328-H350, C358-H380, C388- | |
| | | | | | H409 | |
| 4 | 7503517CD1 | 422 | S89 S193 S339 T55 | | ADP-glucose pyrophosphorylase proteins BL00808: | BLIMPS_{—} |
| | | | T163 T367 | | A5-P24, V101-K134, G335-V366 | BLOCKS |
| | | | | | TRANSLATION INITIATION FACTOR EIF2B | BLAST_{—} |
| | | | | | GAMMA SUBUNIT GDPGTP EXCHANGE | PRODOM |
| | | | | | AMINO ACID BIOSYNTHESIS REGULATION | |
| | | | | | PD105480: S212-E30 | |
| | | | | | TRANSLATION INITIATION FACTOR EIF2B | BLAST_{—} |
| | | | | | GAMMA SUBUNIT GDPGTP EXCHANGE | PRODOM |
| | | | | | AMINO ACID BIOSYNTHESIS PD022735: P141- | |
| | | | | | K189; K189-S211 | |
| | | | | | Rgd: R256-D258 | MOTIFS |
| 5 | 7500014CD1 | 142 | S2 S12 S35 S60 | N130 | | |
| | | | S107 S116 T48 | | | |
| 6 | 7501365CD1 | 433 | S24 S51 S55 S195 | N79 N361 | signal_cleavage: M1-A18 | SPSCAN |
| | | | S222 S223 S379 | | | |
| | | | T178 T197 | | | |
| | | | | | Signal Peptide: M1-A18; M1-Y20; M1-S24 | HMMER |
| | | | | | PROTEIN CASP CARTILAGE ASSOCIATED | BLAST_{—} |
| | | | | | PRECURSOR SIGNAL NUCLEOLAR | PRODOM |
| | | | | | AUTOANTIGEN NO55 NUCLEAR ANTIGEN | |
| | | | | | PD023886: G17-E276 | |
| | | | | | CASP CARTILAGE ASSOCIATED PROTEIN | BLAST_{—} |
| | | | | | PRECURSOR SIGNAL PD155949: L279-R337 | PRODOM |
| 7 | 7503540CD1 | 1450 | S51 S122 S126 | N64 N495 | FHA domain: I23-G90 | HMMER_PFAM |
| | | | S135 S239 S309 | N516 N618 | | |
| | | | S313 S356 S379 | N670 N814 | | |
| | | | S391 S482 S538 | N1045 | | |
| | | | S559 S634 S667 | | | |
| | | | S699 S709 S731 | | | |
| | | | S788 S835 S860 | | | |
| | | | S903 S914 | | | |
| | | | S944 S950 S961 | | Zinc finger, C2H2 type, domain proteins BL00028: | BLIMPS_{—} |
| | | | S968 S988 S1014 | | H387-H403 | BLOCKS |
| | | | S1047 S1064 S1067 | | | |
| | | | S1079 S1096 S1100 | | | |
| | | | S1146 S1212 S1273 | | | |
| | | | S1291 S1335 S1426 | | | |
| | | | T80 T258 T293 | | | |
| | | | T350 T377 T395 | | | |
| | | | T416 T497 T526 | | | |
| | | | T546 T566 T611 | | | |
| | | | T614 T636 T675 | | | |
| | | | T682 T698 T722 | | | |
| | | | T850 T855 T925 | | | |
| | | | T960 T1036 T1041 | | | |
| | | | T1131 T1216 | | | |
| | | | T1267 T1271 Y78 | | | |
| | | | Y1157 | | | |
| 8 | 7504326CD1 | 647 | S31 S37 S96 S131 | N103 N377 | WW domain: S131-P160 | HMMER_PFAM |
| | | | S142 S155 S202 | N415 N529 | | |
| | | | S308 S379 S446 | N533 N536 | | |
| | | | S452 S511 S520 | N539 N546 | | |
| | | | S548 T83 T224 | | | |
| | | | T303 T309 T620 | | | |
| | | | Y25 Y129 Y232 | | | |
| | | | | | WW domain signature PR00403: Y146-P160, S131- | BLIMPS_{—} |
| | | | | | K144 | PRINTS |
| | | | | | do MUCIN; MUC5; TRACHEOBRONCHIAL; | BLAST_DOMO |
| | | | | | DM05454\|S55316\|1-317: P249-T541 | |
| | | | | | Rgd: R19-D21 | MOTIFS |
| | | | | | Ww_Domain_1: W135-P160 | MOTIFS |
| 9 | 7504388CD1 | 195 | S64 S111 S154 T22 | | Rgd: R23-D25 | MOTIFS |
| | | | T41 | | | |
| 10 | 2828380CD1 | 781 | S17 S49 S127 S142 | N118 N164 | Zinc finger, C2H2 type: Y353-H375, C409-H431, | HMMER_PFAM |
| | | | S254 S307 S338 | N339 N675 | Y549-H571, Y493-H515, Y437-H459, Y297-H319, | |
| | | | S391 S395 S419 | N777 | Y577-H599, Y465-H487, Y213-H235, Y633-H655, | |
| | | | S423 S531 S643 | | Y717-H739, Y745-H767, Y661-H683, F325-H347, | |
| | | | S674 S740 T8 T120 | | Y605-H627, Y521-H543, Y381-H403, Y185-H207, | |
| | | | T166 T209 T333 | | Y241-H263, Y269-H291, Y689-H711 | |
| | | | T436 T669 T773 | | | |
| | | | Y161 | | | |
| | | | | | KRAB box: L7-E67 | HMMER_PFAM |
| | | | | | Zinc finger, C2H2 type, domain proteins BL00028: | BLIMPS_{—} |
| | | | | | C327-H343 | BLOCKS |
| | | | | | PROTEIN ZINC-FINGER META PD00066: H595- | BLIMPS_{—} |
| | | | | | C607 | PRODOM |
| | | | | | PROTEIN ZINC FINGER ZINC PD01066: F9-G47 | BLIMPS_{—} |
| | | | | | | PRODOM |
| | | | | | PROTEIN ZINC FINGER METAL BINDING DNA | BLAST_{—} |
| | | | | | BINDING ZINC FINGER PATERNALLY | PRODOM |
| | | | | | EXPRESSED ZNFINGER PW1 PD017719: G461- | |
| | | | | | H711 | |
| | | | | | HYPOTHETICAL ZINC FINGER PROTEIN | BLAST_{—} |
| | | | | | B03B8.4 IN CHROMOSOME III ZINC FINGER | PRODOM |
| | | | | | DNA BINDING METAL BINDING NUCLEAR | |
| | | | | | PD149420: E462-H735, I374-H651 | |
| | | | | | MYELOBLAST KIAA0211 ZINC FINGER METAL | BLAST_{—} |
| | | | | | BINDING DNA BINDING PD149061: K494-H679 | PRODOM |
| | | | | | ZINC FINGER DNA BINDING PROTEIN METAL | BLAST_{—} |
| | | | | | BINDING NUCLEAR ZINC FINGER | PRODOM |
| | | | | | TRANSCRIPTION REGULATION REPEAT | |
| | | | | | PD000072: K519-C582 | |
| | | | | | ZINC FINGER, C2H2 TYPE, DOMAIN DM00002 | BLAST_DOMO |
| | | | | | \|P52743\|31-93: L368-H431 | |
| | | | | | \|Q05481\|789-829: R596-Q637, Q484-E525, R512- | |
| | | | | | E553 | |
| | | | | | \|Q05481\|831-885: C358-E413 | |
| | | | | | KRAB BOX DOMAIN DM00605\|Q03923\|1-75: G5- | BLAST_DOMO |
| | | | | | S49 | |
| | | | | | Zinc finger, C2H2 type, domain: C187-H207, C215- | MOTIFS |
| | | | | | H235, C243-H263, C271-H291, C299-H319, C327- | |
| | | | | | H347, C355-H375, C383-H403, C409-H431, C411- | |
| | | | | | H431, C439-H459, C467-H487, C495-H515, C523- | |
| | | | | | H543, C551-H571, C579-H599, C607-H627, C635- | |
| | | | | | H655, C663-H683, C691-H711, C719-H739, C747- | |
| | | | | | H767 | |
| 11 | 6456919CD1 | 595 | S24 S65 S100 S124 | N12 N39 | Zinc finger, C2H2 type: N172-H194, Y284-H306, | HMMER_PFAM |
| | | | S158 S186 S267 | N118 N122 | Y200-H222, Y256-H278, Y396-H418, Y368-H390, | |
| | | | S270 S307 S382 | N516 | Y424-H448, Y340-H362, Y312-H334, Y458-H480, | |
| | | | T14 T36 T242 | | C228-H250 | |
| | | | T537 Y139 | | | |
| | | | | | KRAB box: V4-Q50 | HMMER_PFAM |
| | | | | | Zinc finger, C2H2 type, domain proteins BL00028: | BLIMPS_{—} |
| | | | | | C398-H414 | BLOCKS |
| | | | | | C2H2-type zinc finger signature PR00048: P367- | BLIMPS_{—} |
| | | | | | S380, L383-G392 | PRINTS |
| | | | | | PROTEIN ZINC-FINGER META PD00066: H386- | BLIMPS_{—} |
| | | | | | C398 | PRODOM |
| | | | | | PROTEIN ZINC FINGER ZINC PD01066: F6-G44 | BLIMPS_{—} |
| | | | | | | PRODOM |
| | | | | | PROTEIN ZINC FINGER METAL BINDING DNA | BLAST_{—} |
| | | | | | BINDING ZINC FINGER PATERNALLY | PRODOM |
| | | | | | EXPRESSED ZNFINGER PW1 PD017719: G224- | |
| | | | | | K506 | |
| | | | | | ZINC FINGER DNA BINDING PROTEIN METAL | BLAST_{—} |
| | | | | | BINDING NUCLEAR ZINC FINGER | PRODOM |
| | | | | | TRANSCRIPTION REGULATION REPEAT | |
| | | | | | PD000072: K366-C429 | |
| | | | | | R30385_2 ZINC FINGER PROTEIN | BLAST_{—} |
| | | | | | TRANSCRIPTION REGULATION DNA BINDING | PRODOM |
| | | | | | REPRESSOR ZINC FINGER METAL BINDING | |
| | | | | | PD030014: K75-E138 | |
| | | | | | KRAB BOX DOMAIN DM00605\|P52737\|1-76: M1- | BLAST_DOMO |
| | | | | | D76 | |
| | | | | | KRAB BOX DOMAIN DM00605\|I49636\|10-85: V4- | BLAST_DOMO |
| | | | | | L66 | |
| | | | | | ZINC FINGER, C2H2 TYPE, DOMAIN DM00002 | BLAST_DOMO |
| | | | | | \|Q05481\|789-829: E359-Q400 | |
| | | | | | \|Q05481\|831-885: C373-E428 | |
| | | | | | Cell attachment sequence: R126-D128 | MOTIFS |
| | | | | | ATP/GTP-binding site motif A (P-loop): A173-T180 | MOTIFS |
| | | | | | Zinc finger, C2H2 type, domain: C174-H194, C202- | MOTIFS |
| | | | | | H222, C228-H250, C230-H250, C286-H306, C314- | |
| | | | | | H334, C342-H362, C370-H390, C398-H418, C426- | |
| | | | | | H448, C460-H480 | |
| 12 | 7502244CD1 | 226 | S143 S192 T59 | | BED zinc finger: S37-R89 | HMMER_PFAM |
| | | | | | PHOSPHATE AMINOTRANSFERA PD00040: R49- | BLIMPS_{—} |
| | | | | | H56 | PRODOM |
| 13 | 7498718CD1 | 548 | S23 S123 S189 | N16 N121 | KRAB box: V22-E84 | HMMER_PFAM |
| | | | S294 S321 S405 | N150 N247 | | |
| | | | S433 S545 T32 T66 | N462 | | |
| | | | T186 T347 T355 | | | |
| | | | T515 Y311 | | | |
| | | | | | Zinc finger, C2H2 type: Y451-H473, Y395-H417, | HMMER_PFAM |
| | | | | | Y423-H445, Y311-H333, Y507-H529, Y479-H501, | |
| | | | | | Y339-H361, Y367-H389, F209-H231 | |
| | | | | | Zinc finger, C2H2 type, domain proteins BL00028: | BLIMPS_{—} |
| | | | | | C397-H413 | BLOCKS |
| | | | | | C2H2-type zinc finger signature PR00048: P394- | BLIMPS_{—} |
| | | | | | K407, L438-G447 | PRINTS |
| | | | | | PROTEIN ZINC-FINGER META PD00066: H441- | BLIMPS_{—} |
| | | | | | C453 | PRODOM |
| | | | | | PROTEIN ZINC FINGER ZINC PD01066: F24-G62 | BLIMPS_{—} |
| | | | | | | PRODOM |
| | | | | | PROTEIN ZINC FINGER METAL BINDING DNA | BLAST_{—} |
| | | | | | BINDING ZINC FINGER PATERNALLY | PRODOM |
| | | | | | EXPRESSED ZNFINGER PW1 PD017719: P310- | |
| | | | | | Y544 | |
| | | | | | ZINC FINGER METAL BINDING DNA BINDING | BLAST_{—} |
| | | | | | PROTEIN FINGER ZINC NUCLEAR REPEAT | PRODOM |
| | | | | | TRANSCRIPTION REGULATION PD001562: V22- | |
| | | | | | E84 | |
| | | | | | ZINC FINGER DNA BINDING PROTEIN METAL | BLAST_{—} |
| | | | | | BINDING NUCLEAR ZINC FINGER | PRODOM |
| | | | | | TRANSCRIPTION REGULATION REPEAT | |
| | | | | | PD000072: K449-C512, K365-C428 | |
| | | | | | ZINC FINGER PROTEIN 186 ZINC FINGER | BLAST_{—} |
| | | | | | METAL BINDING DNA BINDING NUCLEAR | PRODOM |
| | | | | | PD048826: M223-P262 | |
| | | | | | KRAB BOX DOMAIN DM00605 | BLAST_DOMO |
| | | | | | \|I48689\|11-85: E20-L87 | |
| | | | | | \|P51786\|24-86: V22-W81 | |
| | | | | | \|P51523\|5-79: E20-I82 | |
| | | | | | \|P17097\|1-76: L19-E84 | |
| | | | | | ATP/GTP-binding site motif A (P-loop): G340-T347, | MOTIFS |
| | | | | | A536-T543 | |
| | | | | | Zinc finger, C2H2 type, domain: C313-H333, C341- | MOTIFS |
| | | | | | H361, C369-H389, C397-H417, C425-H445, C453- | |
| | | | | | H473, C481-H501, C509-H529 | |
| 14 | 6259308CD1 | 264 | S15 S25 S118 S125 | N23 | RNA recognition motif. (a.k.a. RRM, RBD, or: L115- | HMMER_PFAM |
| | | | S149 S190 S246 | | I185 | |
| | | | T138 T222 T226 | | | |
| | | | | | PROTEIN NUCLEAR RIBONUCL PD02784: A107- | BLIMPS_{—} |
| | | | | | A143, S149-Q191 | PRODOM |
| | | | | | TRANSCRIPTIONAL COACTIVATOR ALY ALY | BLAST_{—} |
| | | | | | PD056100: Q50-K114 | PRODOM |
| | | | | | PROTEIN F23B2.6 C01F6.5 M18.7 (C. ELEGANS | BLAST_{—} |
| | | | | | PROTEIN) PD016912: K114-R260 | PRODOM |
| 15 | 7504104CD1 | 611 | S32 S48 S102 S130 | N184 N214 | Helix-loop-helix DNA-binding domain: R509-E562 | HMMER_PFAM |
| | | | S143 S209 S324 | N241 N278 | | |
| | | | S455 S468 S490 | | | |
| | | | S541 T12 T75 T81 | | | |
| | | | T104 T393 T423 | | | |
| | | | Y77 | | | |
| | | | | | Myc-type, ‘helix-loop-helix’ dimerization domain | BLIMPS_{—} |
| | | | | | proteins BL00038: E517-G532, D542-E562 | BLOCKS |
| | | | | | Myc-type, ‘helix-loop-helix’ dimerization domain | PROFILESCAN |
| | | | | | signature: A527-E582 | |
| | | | | | PROTEIN TRANSCRIPTION DNA BINDING | BLAST_{—} |
| | | | | | REGULATION NUCLEAR FACTOR | PRODOM |
| | | | | | ALTERNATIVE IMMUNOGLOBULIN SPLICING | |
| | | | | | ITF2 PD005047: Q323-E508; M220-L322 | |
| | | | | | PROTEIN TRANSCRIPTION DNA BINDING | BLAST_{—} |
| | | | | | FACTOR REGULATION NUCLEAR | PRODOM |
| | | | | | ALTERNATIVE IMMUNOGLOBULIN SPLICING | |
| | | | | | ITF2 PD006397: M25-T222 | |
| | | | | | PROTEIN TRANSCRIPTION FACTOR | BLAST_{—} |
| | | | | | REGULATION DNA BINDING NUCLEAR | PRODOM |
| | | | | | ALTERNATIVE IMMUNOGLOBULIN SPLICING | |
| | | | | | ITF2 PD006396: F151-P219 | |
| | | | | | PROTEIN TRANSCRIPTION DNA BINDING | BLAST_{—} |
| | | | | | REGULATION NUCLEAR FACTOR | PRODOM |
| | | | | | ALTERNATIVE IMMUNOGLOBULIN SPLICING | |
| | | | | | ENHANCER PD005628: Q563-M611 | |
| | | | | | HUMAN TRANSCRIPTION FACTOR 3 DM01610 | BLAST_DOMO |
| | | | | | \|P15881\|232-507: L197-K473 | |
| | | | | | \|Q99081\|269-540: L197-D475 | |
| | | | | | \|S23391\|260-513: L197-D471 | |
| | | | | | MYC-TYPE, ‘HELIX-LOOP-HELIX’ | BLAST_DOMO |
| | | | | | DIMERIZATION DOMAIN DM00051\|P15881\|509- | |
| | | | | | 637: L474-H607 | |
| | | | | | Myc-type, ‘helix-loop-helix’ dimerization domain | MOTIFS |
| | | | | | signature: T546-L561 | |
| 16 | 7504121CD1 | 386 | S42 S139 S313 | N59 N128 | HMG (high mobility group) box: I271-S339 | HMMER_PFAM |
| | | | S339 S366 T78 | | | |
| | | | T159 T247 T294 | | | |
| | | | T369 Y102 | | | |
| | | | | | TRANSCRIPTION PROTEIN DN PD02448: N276- | BLIMPS_{—} |
| | | | | | R314, E315-G362 | PRODOM |
| | | | | | FACTOR TRANSCRIPTION PROTEIN | BLAST_{—} |
| | | | | | LYMPHOID ENHANCER BINDING | PRODOM |
| | | | | | DNABINDING NUCLEAR REGULATION | |
| | | | | | 3DSTRUCTURE PD009503: M1-M126 | |
| | | | | | FACTOR TRANSCRIPTION PROTEIN | BLAST_{—} |
| | | | | | DNABINDING NUCLEAR REGULATION | PRODOM |
| | | | | | LYMPHOID ENHANCER BINDING | |
| | | | | | 3DSTRUCTURE PD007419: N128-P245 | |
| | | | | | FACTOR TRANSCRIPTION LYMPHOID | BLAST_{—} |
| | | | | | ENHANCER BINDING DNABINDING NUCLEAR | PRODOM |
| | | | | | PROTEIN REGULATION 3DSTRUCTURE | |
| | | | | | PD155139: A234-I271 | |
| | | | | | do LEF-1S; DM03940 | BLAST_DOMO |
| | | | | | \|P27782\|114-284: M116-P220 | |
| | | | | | \|S42128\|1-171: M116-P220 | |
| | | | | | HMG BOX DM00056 | BLAST_DOMO |
| | | | | | \|S42128\|173-246: Q260-L334 | |
| | | | | | \|P27782\|286-359: Q260-L334 | |
| 17 | 5635695CD1 | 807 | S5 S71 S119 S289 | N361 N417 | Pyrokinins proteins BL00539: F528-L532 | BLIMPS_{—} |
| | | | S291 S322 S372 | N496 N501 | | BLOCKS |
| | | | S373 S386 S503 | N566 N723 | | |
| | | | S512 S522 S538 | N735 | | |
| | | | S545 S558 S622 | | | |
| | | | S650 T43 T156 | | | |
| | | | T203 T215 T220 | | | |
| | | | T246 T257 T467 | | | |
| | | | T544 T567 T674 | | | |
| | | | Y231 | | | |
| 18 | 7503983CD1 | 257 | S69 S82 S138 T49 | N67 | EF-1 guanine nucleotide exchange domain: K171- | HMMER_PFAM |
| | | | T123 T240 Y18 | | I257 | |
| | | | | | Elongation factor 1 beta/beta′/delta chain proteins | BLIMPS_{—} |
| | | | | | BL00824: F242-I257, L70-L84, D128-A147, K161- | BLOCKS |
| | | | | | Q198, L199-D233 | |
| | | | | | ELONGATION FACTOR PROTEIN | BLAST_{—} |
| | | | | | BIOSYNTHESIS 1BETA EF1BETA | PRODOM |
| | | | | | PHOSPHORYLATION 1DELTA EF1DELTA BETA | |
| | | | | | PD002592: E129-I257 | |
| | | | | | ELONGATION FACTOR 1DELTA EF1DELTA | BLAST_{—} |
| | | | | | PROTEIN BIOSYNTHESIS P36 FACTOR1 DELTA | PRODOM |
| | | | | | FACTOR1D PD010654: A36-P103, L6-G42 | |
| | | | | | ELONGATION FACTOR 1 BETA/BETA′/DELTA | BLAST_DOMO |
| | | | | | CHAIN | |
| | | | | | DM01052\|P29692\|97-280: V73-I257 | |
| | | | | | DM01052\|P29693\|75-264: V73-I257 | |
| | | | | | DM01052\|P29522\|20-220: T105-I257 | |
| | | | | | DM01052\|P32192\|26-236: Q106-1257 | |
| | | | | | Elongation factor 1 beta/beta′/delta chain signature 1: | MOTIFS |
| | | | | | E129-G137 | |
| | | | | | Elongation factor 1 beta/beta′/delta chain signature 2: | MOTIFS |
| | | | | | V246-I257 | |
| | | | | | Leucine zipper pattern: L56-L77, L63-L84, L70-L91 | MOTIFS |
| 19 | 7503476CD1 | 113 | S95 T8 | N58 | Ribosomal protein L15: K75-G107 | HMMER_PFAM |
| | | | | | Ribosomal protein L15 signature: K75-A114 | PROFILESCAN |
| | | | | | Ribosomal protein L15 proteins BL00475: K7-R21, | BLIMPS_{—} |
| | | | | | K27-G36, I66-L82, P86-G107 | BLOCKS |
| | | | | | RIBOSOMAL PROTEIN L27A 60S L29 L28 L22 | BLAST_{—} |
| | | | | | CRP1 YL24 RP62 PD002840: M1-Y48 | PRODOM |
| | | | | | RIBOSOMAL PROTEIN L15 | BLAST_DOMO |
| | | | | | DM00524\|S55914\|3-145: H19-L111, S3-Y48 | |
| | | | | | DM00524\|P41092\|4-146: R6-Y48, R12-A55 | |
| | | | | | DM00524\|P49637\|3-143: S3-Y48, R4-L111 | |
| | | | | | DM00524\|P48160\|3-145: H19-L111, S3-Y48 | |
| | | | | | Ribosomal protein L15 signature: K75-G106 | MOTIFS |
| 20 | 7504023CD1 | 204 | S194 T8 T40 T93 | | 60s Acidic ribosomal protein: L104-D204 | HMMER_PFAM |
| | | | | | RIBOSOMAL PROTEIN ACIDIC P0 60S | BLAST_{—} |
| | | | | | PHOSPHORYLATION L10E HOMOLOG L10 | PRODOM |
| | | | | | ISOLOG PD002352: V56-V124 | |
| | | | | | RAT ACIDIC RIBOSOMAL PROTEIN P0 | BLAST_DOMO |
| | | | | | DM00904\|P19889\|1-315: N34-L104, G105-F203, M1- | |
| | | | | | M50 | |
| | | | | | DM00904\|P50346\|1-318: G105-F203, I29-L104, P2- | |
| | | | | | G78 | |
| | | | | | DM00904\|P05317\|1-310: L21-G117, I103-F203, K10- | |
| | | | | | V56 | |
| | | | | | DM00904\|P22685\|1-303: G105-F203, K10-G117, | |
| | | | | | K10-G36 | |
| 21 | 7504128CD1 | 144 | T18 T38 T84 T107 | N82 | Ribosomal protein L11: L35-D123, V13-P34 | HMMER_PFAM |
| | | | | | Ribosomal protein L11 proteins BL00359: N90- | BLIMPS_{—} |
| | | | | | D123, L35-K75 | BLOCKS |
| | | | | | Ribosomal protein L11 signature: V89-A143 | PROFILESCAN |
| | | | | | RIBOSOMAL PROTEIN L11 | BLAST_DOMO |
| | | | | | DM00681\|P30050\|6-149: L35-D129, D6-K40 | |
| | | | | | DM00681\|P17079\|6-149: E21-D129 | |
| | | | | | DM00681\|P54030\|1-143: L35-D129, K11-Q44 | |
| | | | | | Ribosomal protein L11 signature: K109-D123 | MOTIFS |
| 22 | 4529338CD1 | 355 | S6 S11 S150 S210 | N80 N84 | QUAKING PROTEIN HOMOLOG KH DOMAIN | BLAST_{—} |
| | | | S313 T87 T131 | N223 | RNA BINDING QKI7B QKI7 PD032709: D228- | PRODOM |
| | | | T178 T225 T300 | | L325 | |
| | | | | | PROTEIN PHOSPHOPROTEIN P62 ZFM1 | BLAST_{—} |
| | | | | | TYROSINE PUTATIVE TRANSCRIPTION | PRODOM |
| | | | | | FACTOR NUCLEAR GAPASSOCIATED | |
| | | | | | PD149659: P100-Q159 | |
| | | | | | PROTEIN ZFM1 PUTATIVE PHOSPHOPROTEIN | BLAST_{—} |
| | | | | | P62 TRANSCRIPTION FACTOR NUCLEAR KH | PRODOM |
| | | | | | RNA PD002056: R161-R227 | |
| | | | | | PROTEIN KH RNA BINDING QUAKING | BLAST_{—} |
| | | | | | FEMALE GERMLINE-SPECIFIC TUMOR | PRODOM |
| | | | | | SUPPRESSOR GLD1 PD008249: E28-D96 | |
| | | | | | PHOSPHOPROTEIN; P62; GAP; RAS-GAP | BLAST_DOMO |
| | | | | | DM02127\|A38219\|82-278: K32-G224 | |
| | | | | | DM02127\|I49140\|82-278: K32-G224 | |
| | | | | | DM02127\|P42083\|473-667: N119-R227 | |
| | | | | | DM02127\|S64953\|79-278: P95-P234 | |
| 23 | 7503460CD1 | 143 | S28 S29 T23 | | Surp module: R50-E103 | HMMER_PFAM |
| | | | | | SPLICING PROTEIN MRNA NUCLEAR FACTOR | BLAST_{—} |
| | | | | | SPLICEOSOME REPEAT PREMRNA PRP21 | PRODOM |
| | | | | | PUTATIVE PD009917: E48-P129 | |
| | | | | | SPLICEOSOME ASSOCIATED PROTEIN 114 SAP | BLAST_{—} |
| | | | | | SF3A120 MRNA PROCESSING SPLICING | PRODOM |
| | | | | | NUCLEAR REPEAT PD125875: T17-P47 | |
| 24 | 5466630CD1 | 1048 | S60 S62 S68 S170 | N214 N229 | DEAD/DEAH box helicase: R208-E324, E149-T173 | HMMER_PFAM |
| | | | S186 S206 S216 | | | |
| | | | S401 S456 S545 | | | |
| | | | S713 S722 S740 | | | |
| | | | S888 T7 T54 T284 | | | |
| | | | T301 T338 T592 | | | |
| | | | T699 T879 Y873 | | | |
| | | | | | Helicase conserved C-terminal domain: E471-E567 | HMMER_PFAM |
| | | | | | DEAH-box subfamily ATP-dependent helicases | BLIMPS_{—} |
| | | | | | proteins BL00690: G166-Q175, T195-E212, V260- | BLOCKS |
| | | | | | S269 | |
| | | | | | DEAD and DEAH box families ATP-dependent | PROFILESCAN |
| | | | | | helicases signatures: I236-A287 | |
| | | | | | COSMID 30B8 PUTATIVE ATP-DEPENDENT | BLAST_{—} |
| | | | | | RNA HELICASE C06E1.10 CHROMOSOME III | PRODOM |
| | | | | | PROTEIN PD041384: K731-H1048 | |
| | | | | | POLYPROTEIN PROTEIN HELICASE GENOME | BLAST_{—} |
| | | | | | RNA CONTAINS: NUCLEAR ENVELOPE ATP- | PRODOM |
| | | | | | BINDING NONSTRUCTURAL PD000440: V481- | |
| | | | | | A578, G84-P311, P501-L574 | |
| | | | | | PUTATIVE ATP-DEPENDENT RNA HELICASE | BLAST_{—} |
| | | | | | PROTEIN ATP-BINDING RNA-BINDING | PRODOM |
| | | | | | C06E1.10 CHROMOSOME III PD001244: S325- | |
| | | | | | K416 | |
| | | | | | HELICASE RNA ATP-BINDING PROTEIN ATP- | BLAST_{—} |
| | | | | | DEPENDENT NUCLEAR SPLICING MRNA | PRODOM |
| | | | | | PROCESSING PREMRNA PD001259: C571-E716 | |
| | | | | | DEAH-BOX SUBFAMILY ATP-DEPENDENT | BLAST_DOMO |
| | | | | | HELICASES | |
| | | | | | DM00649\|P34305\|227-973: E135-K881 | |
| | | | | | DM00649\|S53058\|382-1163: E135-P854, D802-I876 | |
| | | | | | DM00649\|A56236\|555-1160: E138-L380, P473- | |
| | | | | | E716, R843-Y883, P807-D826, Q553-M603 | |
| | | | | | DM00649\|P34498\|432-1038: M136-K382, G470- | |
| | | | | | F704, Q764-Y883 | |
| | | | | | ATP/GTP-binding site motif A (P-loop): G166-T173 | MOTIFS |
| 25 | 7503474CD1 | 294 | S130 S269 T101 | N247 N263 | Signal_cleavage: M1-G60 | SPSCAN |
| | | | T123 T168 T183 | | | |
| | | | T233 | | | |
| | | | | | Sir2 family: D66-P160, G52-R65 | HMMER_PFAM |
| | | | | | PROTEIN SIR2 TRANSCRIPTION REGULATION | BLAST_{—} |
| | | | | | REPRESSOR DNA-BINDING ZINC-FINGER | PRODOM |
| | | | | | NUCLEAR REGULATORY SILENT PD002659: | |
| | | | | | P26-L208 | |
| | | | | | Aminotransferases class-II pyridoxal-phosphate | MOTIFS |
| | | | | | attachment site: T106-A115 | |
| 26 | 7503498CD1 | 280 | S54 S76 S85 S142 | | RIBONUCLEASE P PROTEIN SUBUNIT P40 | BLAST_{—} |
| | | | S151 S166 T40 | | PD182342: I27-P280, M1-G30 | PRODOM |
| | | | T103 T130 Y256 | | | |
| 27 | 7504119CD1 | 288 | S4 S124 S159 | N53 N157 | Ribosomal RNA adenine dimethylase: Q35-V228 | HMMER_PFAM |
| | | | S273 T9 T17 T44 | | | |
| | | | T114 Y286 | | | |
| | | | | | TRANSFERASE METHYLTRANSFERASE RRNA | BLAST_{—} |
| | | | | | RESISTANCE PROTEIN ADENINE ANTIBIOTIC | PRODOM |
| | | | | | N6METHYLTRANSFERASE B PLASMID | |
| | | | | | PD000922: Q35-E276 | |
| | | | | | RIBOSOMAL RNA ADENINE DIMETHYLASES | BLAST_DOMO |
| | | | | | DM00429\|P37468\|16-288: K31-E280 | |
| | | | | | DM00429\|P44749\|5-270: A29-L264 | |
| | | | | | DM00429\|P06992\|5-265: K104-L264 | |
| | | | | | DM00429\|P47701\|1-255: I138-R265, A30-N157 | |
| | | | | | Immunoglobulins and major histocompatibility | MOTIFS |
| | | | | | complex proteins signature: Y202-H208 | |
| 28 | 71532805CD1 | 244 | S39 S42 S145 T26 | | Ribosomal protein L30p/L7e: | HMMER_PFAM |
| | | | T101 Y151 | | K84-V136 | |
| | | | | | Ribosomal protein L30 BL00634: V89-G139 | BLIMPS_{—} |
| | | | | | | BLOCKS |
| | | | | | Ribosomal protein L30 signature: | PROFILESCAN |
| | | | | | V88-A137 | |
| | | | | | RIBOSOMAL PROTEIN 60S L7 MULTIGENE | BLAST_{—} |
| | | | | | FAMILY RNABINDING REPEAT L7A L7B | PRODOM |
| | | | | | PD149881 A137-R242, PD005715: K7-E82 | |
| | | | | | RAT RIBOSOMAL PROTEIN L7 DM02153 | BLAST_DOMO |
| | | | | | P11874\|30-245: F47-N244, P25457\|33-249: A32- | |
| | | | | | M243, P05737\|25-242: E30-N244, P11874\|30-245: | |
| | | | | | F47-N244 | |
| | | | | | Ribosomal L30 Motif: I104-V135 | MOTIFS |
| | | | | | Eukaryotic thiol (cysteine) proteases histidine active | MOTIFS |
| | | | | | site: | |
| | | | | | L186-H196 | |
| 29 | 5502992CD1 | 1953 | S9 S21 S58 S131 | N46 N440 | Helicase conserved C-terminal domain: | HMMER_PFAM |
| | | | S335 S373 S568 | N589 N663 | D535-G619 | |
| | | | S572 S644 S658 | N734 N893 | | |
| | | | S671 S739 S792 | N1049 | | |
| | | | S796 S804 S881 | | | |
| | | | S914 S937 S978 | | | |
| | | | S999 S1093 S1131 | | | |
| | | | S1315 S1380 S1418 | | | |
| | | | S1441 S1443 S1465 | | | |
| | | | S1466 S1470 S1545 | | | |
| | | | S1550 S1551 S1595 | | | |
| | | | S1786 S1787 S1929 | | | |
| | | | S1934 S1947 T35 | | | |
| | | | T244 T252 T297 | | | |
| | | | T355 T419 T459 | | | |
| | | | T591 T711 T770 | | | |
| | | | T777 T782 T847 | | | |
| | | | T995 T1019 T1118 | | | |
| | | | T1171 T1362 | | | |
| | | | T1395 T1409 | | | |
| | | | T1576 T1587 | | | |
| | | | T1900 Y118 Y1068 | | | |
| | | | | | SNF2 and others N-terminal domain Y186-F473 | HMMER_PFAM |
| | | | | | Chromo domain proteins BL00598: Y118-V139 | BLIMPS_{—} |
| | | | | | | BLOCKS |
| | | | | | ATP-Binding Nucleoside PD02191: C316-C330, | BLIMPS_{—} |
| | | | | | L336-H364, C596-S621 | PRODOM |
| | | | | | O61845_CAEEL // T04D1.4 PROTEIN PD145655: | BLAST_{—} |
| | | | | | L679-K1031, W1170-S1328, W1111-T1171, P1527- | PRODOM |
| | | | | | W1548 | |
| | | | | | (142) NTP1(5) O22731(3) CHD1(3) // PROTEIN | BLAST_{—} |
| | | | | | HELICASE ATPBINDING NUCLEAR | PRODOM |
| | | | | | DNABINDING ZINCHNGER DNA | |
| | | | | | TRANSCRIPTION REPAIR I PD000441: K385- | |
| | | | | | I473, S304-Q389, Y186-E224, Y177-E249, L235- | |
| | | | | | S267, I667-K696 | |
| | | | | | O61845_CAEEL // T04D1.4 PROTEIN PD126894: | BLAST_{—} |
| | | | | | E36-S174, V5-E39 | PRODOM |
| | | | | | HELICASE ATP-BINDING RNA-BINDING | BLAST_{—} |
| | | | | | INITIATION FACTOR ATP-DEPENDENT | PRODOM |
| | | | | | EUKARYOTIC BIOSYNTHESIS DNA-BINDING | |
| | | | | | PD000085: N203-L366 | |
| | | | | | N203-L366 | |
| | | | | | ATP NP_BIND DM00266\|P51531\|741-1166: I205- | BLAST_DOMO |
| | | | | | V626 | |
| | | | | | DM00266\|P32657\|397-815 ATP NP_BIND I205- | |
| | | | | | V626 | |
| | | | | | DM00266\|P40201\|500-906 ATP NP_BIND C204- | |
| | | | | | V626 | |
| | | | | | DM00266\|P28370\|126-540 ATP NP_BIND I205- | |
| | | | | | V626 | |
| | | | | | Cell attachment sequence: | MOTIFS |
| | | | | | R867-D869, R1144-D1146 | |
| 30 | 7503828 | 1099 | S115 S219 S224 | N46 N173 | Myb-like DNA-binding domain myb_DNA_binding: | HMMER_PFAM |
| | | | S267 S273 S286 | N398 N416 | T598-L642 | |
| | | | S302 S327 S367 | N454 N857 | | |
| | | | S682 S695 S754 | | | |
| | | | S806 S810 S813 | | | |
| | | | S841 T22 T45 | | | |
| | | | T166 T247 T276 | | | |
| | | | T363 T376 T378 | | | |
| | | | T391 T472 T583 | | | |
| | | | T602 T631 T726 | | | |
| | | | T743 T744 T847 | | | |
| | | | T911 Y648 Y922 | | | |
| | | | | | PD025015 O76489(1) P97496(1) Q92922(1) // A | BLAST_{—} |
| | | | | | SWI/SNF ASSOCIATED COMPLEX SUBUNIT | PRODOM |
| | | | | | BRAHMA PROTEIN RELATED MATRIX ACTIN | |
| | | | | | R4-E337 | |
| | | | | | PD007613 // PROTEIN SWI/SNF COMPLEX | BLAST_{—} |
| | | | | | SUBUNIT A BAF170 CHROMOSOME I | PRODOM |
| | | | | | ASSOCIATED SIMILAR D338-A554 | |
| | | | | | PD023971 O76489(1) P97496(1) Q92922(1) // A | BLAST_{—} |
| | | | | | SWI/SNF ASSOCIATED COMPLEX SUBUNIT | PRODOM |
| | | | | | BRAHMA PROTEIN RELATED MATRIX ACTIN | |
| | | | | | V688-L858 | |
| | | | | | PD006967 // PROTEIN SWI/SNF COMPLEX | BLAST_{—} |
| | | | | | SUBUNIT A BAF170 CHROMOSOME I | PRODOM |
| | | | | | ASSOCIATED SIMILAR Q551-H638 | |
| | | | | | FIBRILLAR COLLAGEN CARBOXYL- | BLAST_{—} |
| | | | | | TERMINAL DM00019\|P17656\|108-273 Q960- | PRODOM |
| | | | | | G1090 | |
| | | | | | DM00019\|P34687\|106-271 Q959-P1098 | |
| | | | | | DM00019\|P08124\|103-269 Q959-S1088, P963- | |
| | | | | | P1098, P963-P1097 | |
| | | | | | PROLINE-RICH PROTEIN DM03894\|A39066\|1-159 | BLAST_{—} |
| | | | | | L939-Q1099 | PRODOM |
| 31 | 2647325CD1 | 203 | S78 S106 S180 | | Zinc finger, C2H2 type: | HMMER_PFAM |
| | | | | | F68-H90, Y37-H59, Y166-H188, F96-H118, H124- | |
| | | | | | H146 | |
| | | | | | signal_cleavage: | SPSCAN |
| | | | | | M1-G16 | |
| | | | | | Zinc finger, C2H2 type BL00028: C168-H184 | BLIMPS_{—} |
| | | | | | | BLOCKS |
| | | | | | Protein Zinc-finger metal binding domain PD00066: | BLIMPS_{—} |
| | | | | | H114-C126 | PRODOM |
| | | | | | ZINC-FINGER DNA-BINDING METAL-BINDING | BLAST_{—} |
| | | | | | NUCLEAR TRANSCRIPTION REPEAT | PRODOM |
| | | | | | REGULATION FACTOR PD017719 P36-G191, | |
| | | | | | PD000072: F68-C129 | |
| | | | | | ZINC FINGER, C2H2 TYPE, DOMAIN | BLAST_DOMO |
| | | | | | DM00002\|P08042\|314-358: C73-H118, P17097\|353- | |
| | | | | | 390: Q87-K122 | |
| | | | | | Zinc finger, C2H2 type, domain: | MOTIFS |
| | | | | | C39-H59, C70-H90, C98-H118, C126-H146, C168- | |
| | | | | | H188 | |
| 32 | 7495416CD1 | 317 | S101 S129 T77 | | Zinc finger, C2H2 type: | HMMER_PFAM |
| | | | T142 T306 T308 | | Y119-H141, H63-H85, F91-H113, Y203-H225, Y147- | |
| | | | Y147 | | H169, Y175-H197, H231-H253, Y259-H281 | |
| | | | | | Zinc finger, C2H2 type BL00028: C121-H137 | BLIMPS_{—} |
| | | | | | | BLOCKS |
| | | | | | C2H2 type Zinc finger signature PR00048: P90-K103, | BLIMPS_{—} |
| | | | | | L190-G199 | PRINTS |
| | | | | | Protein Zinc-finger metal binding domain PD00066: | BLIMPS_{—} |
| | | | | | H165-C177 | PRODOM |
| | | | | | ZINC-FINGER DNA-BINDING METAL-BINDING | BLAST_{—} |
| | | | | | NUCLEAR PATERNALLY EXPRESSED | PRODOM |
| | | | | | PD017719: A60-F296, G87-H281, G115-F296 | |
| | | | | | ZINC-FINGER DNA-BINDING METAL-BINDING | BLAST_{—} |
| | | | | | NUCLEAR TRANSCRIPTION REPEAT | PRODOM |
| | | | | | REGULATION FACTOR PD000072: R117-C180 | |
| | | | | | R89-C152, R61-C124, K173-C236, R201-C264, | |
| | | | | | K145-C208 | |
| | | | | | ZINC FINGER, C2H2 TYPE, DOMAIN | BLAST_DOMO |
| | | | | | DM00002\|P17097\|353-390: R194-K229 | |
| | | | | | DM00002\|Q05481\|789-829: R167-D207, R194-C233, | |
| | | | | | R83-E123 | |
| | | | | | DM00002\|P08042\|314-358 C68-H113, C96-H141 | |
| | | | | | DM00002\|Q05481\|831-885 C180-R232 | |
| | | | | | Zinc finger, C2H2 type, domain: | MOTIFS |
| | | | | | C65-H85, C93-H113, C121-H141, C149-H169, C177- | |
| | | | | | H197, C205-H225, C233-H253, C261-H281 | |
| 33 | 8096177CD1 | 579 | S9 S91 S131 S156 | N2 N40 | Zinc finger, C2H2 type: | HMMER_PFAM |
| | | | S161 S189 S254 | N108 N149 | Y520-H542, Y548-H570, Y408-H430, Y464-H486, | |
| | | | S260 S330 S362 | N159 N258 | F380-H402 | |
| | | | S558 T18 T52 | N357 N360 | | |
| | | | T231 T250 T320 | N394 N450 | | |
| | | | T388 T528 Y270 | | | |
| | | | | | KRAB box: V8-E70 | HMMER_PFAM |
| | | | | | Zinc finger, C2H2 type BL00028: C410-H426 | BLIMPS_{—} |
| | | | | | | BLOCKS |
| | | | | | C2H2 type Zinc finger signature PR00048: P407- | BLIMPS_{—} |
| | | | | | S420, L535-G544 | PRINTS |
| | | | | | PROTEIN Zinc Finger PD01066 F10-G48 | BLIMPS_{—} |
| | | | | | | PRODOM |
| | | | | | Protein Zinc-finger metal binding domain PD00066: | BLIMPS_{—} |
| | | | | | H398-C410 | PRODOM |
| | | | | | ZINC-FINGER DNA-BINDING METAL-BINDING | BLAST_{—} |
| | | | | | NUCLEAR TRANSCRIPTION REPEAT | PRODOM |
| | | | | | REGULATION FACTOR PD000072: K434-C497, | |
| | | | | | K490-C553, K406-C469, K518-H570, K378-C441, | |
| | | | | | K462-C525; PD001562: V8-E70; PD033163: D353- | |
| | | | | | K490, C382-K518, V481-V574 | |
| | | | | | ZINC-FINGER DNA-BINDING METAL-BINDING | BLAST_{—} |
| | | | | | NUCLEAR PATERNALLY EXPRESSED | PRODOM |
| | | | | | PD017719: | |
| | | | | | L340-H570, G376-V573, G348-H570, P269-K518, | |
| | | | | | N295-H542 | |
| | | | | | KRAB BOX DOMAIN | BLAST_DOMO |
| | | | | | DM00605\|P52736\|1-72: V8-C77 | |
| | | | | | DM00605\|I48689\|11-85: Q5-K71 | |
| | | | | | DM00605\|P51523\|5-79: Q5-F79 | |
| | | | | | DM00605\|P51786\|24-86: V8-W67 | |
| | | | | | ATP/GTP-binding site motif A (P-loop): G271-S279 | MOTIFS |
| | | | | | Zinc finger, C2H2 type, domain: | MOTIFS |
| | | | | | C382-H402, C410-H430, C438-H458, C466-H486, | |
| | | | | | C494-H514, C522-H542, C550-H570 | |
| 34 | 666763CD1 | 730 | S68 S100 S102 | N244, N283, | MCM family proteins BL00847H: T151-R168, A42- | BLIMPS_{—} |
| | | | S185 S206 S263 | N310, N365, | K96, P123-Q142 | BLOCKS |
| | | | S278 S314 S318 | N449, N611, | | |
| | | | S380 S389 S404 | N632 | | |
| | | | S450 S477 S521 | | | |
| | | | S539 S543 S631 | | | |
| | | | S634 S686 S690 | | | |
| | | | S730 T28 T79 | | | |
| | | | T124 T152 T176 | | | |
| | | | T313 T354 T511 | | | |
| | | | T547 T697 T709 | | | |
| | | | | | REPLICATION DNA CELL DNA-BINDING | BLAST_{—} |
| | | | | | REGULATION ATP-BINDING TRANSCRIPTION | PRODOM |
| | | | | | NUCLEAR FACTOR LICENSING PD001041: R27- | |
| | | | | | K104, M108-M191 | |
| | | | | | MCM2/3/5 FAMILY | BLAST_DOMO |
| | | | | | DM00603\|JC4580\|223-719: R27-G198 | |
| | | | | | DM00603\|P34647\|193-736 L34-M191 | |
| | | | | | DM00603\|P33991\|340-862 R27-D179, T679-K704 | |
| | | | | | DM00603\|P30665\|386-928 R27-V254 | |
| 35 | 7504091CD1 | 315 | S66 S94 S127 T47 | N30 N147 | Ribosomal protein L3: | HMMER_PFAM |
| | | | T142 T174 T203 | N253 | K124-K267, K103-Q123 | |
| | | | | | signal_cleavage: | SPSCAN |
| | | | | | M1-G43 | |
| | | | | | Ribosomal protein L3 proteins BL00474: L99-L109, | BLIMPS_{—} |
| | | | | | F165-G199, G208-N244 | BLOCKS |
| | | | | | Ribosomal protein L3 signature: | PROFILESCAN |
| | | | | | F146-A209 | |
| | | | | | RIBOSOMAL L3 MITOCHONDRIAL 60S | BLAST_{—} |
| | | | | | MITOCHONDRION PD105243: M1-G29; | PRODOM |
| | | | | | PD036323 N30-Q123, K124-E134; PD036320: I250- | |
| | | | | | A315, PD002374: F131-I249, K246-K267 | |
| | | | | | RIBOSOMAL PROTEIN L3 | BLAST_DOMO |
| | | | | | DM00364\|P38515\|9-200: K112-K267 | |
| | | | | | DM00364\|P09001\|105-300 V119-D268, G105-Q123 | |
| | | | | | DM00364\|P49404\|87-295 E130-D268, G105-Q123 | |
| | | | | | DM00364\|P31334\|68-263 G114-D268 | |
| | | | | | Ribosomal protein L3 signature: | MOTIFS |
| | | | | | F165-R188 | |
| 36 | 7503568 | 317 | S35 S154 S217 | N11 N48 | KH domain: R101-G150, E243-G291, R17-G63 | HMMER_PFAM |
| | | | S222 T15 T99 | N89 N140 | | |
| | | | T142 T240 T283 | | | |
| | | | | | KH domain proteins family PF00013: L112-I123 | BLIMPS_{—} |
| | | | | | | PFAM |
| | | | | | PROTEIN NUCLEAR RNABINDING | BLAST_{—} |
| | | | | | RIBONUCLEOPROTEIN DNABINDING REPEAT | PRODOM |
| | | | | | HNRNPE1 POLYCBINDING NUCLEIC ACID | |
| | | | | | PD010726: L194-T240, I151-Q193 | |
| | | | | | RNA BINDING PROTEIN PUTATIVE PRE MRNA | BLAST_{—} |
| | | | | | SPLICING FACTOR PD182839: L14-P180 | PRODOM |
| | | | | | PROTEIN NUCLEAR RNABINDING | BLAST_{—} |
| | | | | | RIBONUCLEOPROTEIN DNABINDING REPEAT | PRODOM |
| | | | | | HNRNPE1 POLYCBINDING | |
| | | | | | PHOSPHORYLATION PROTEIN1 PD151096: P64- | |
| | | | | | R101 | |
| | | | | | KH DOMAIN | BLAST_DOMO |
| | | | | | DM00168\|I48281\|86-167: S86-E168 | |
| | | | | | DM00168\|S58529\|86-167: S86-E168 | |
| | | | | | DM00168\|I48281\|6-84: I6-S85 | |
| | | | | | COMPLEX; NUCLEAR; RIBONUCLEOPROTEIN; | BLAST_DOMO |
| | | | | | HETEROGENEOUS; DM08370\|S58529\|232-328: | |
| | | | | | L194-I289 | |
| 37 | 7504101CD1 | 748 | S83 S129 S203 | | COIL COILED MYOSIN CHAIN ATP-BINDING | BLAST_{—} |
| | | | S206 S224 S274 | | HEAVY FILAMENT MUSCLE REPEAT | PRODOM |
| | | | S294 S493 S526 | | INTERMEDIATE PD000002: | |
| | | | S600 S614 S616 | | Q338-I445, E331-K442, Q338-E444 | |
| | | | S642 T17 T175 | | | |
| | | | T183 T322 T330 | | | |
| | | | T485 | | | |
| | | | | | CHROMATIN ASSEMBLY FACTORI P150 | BLAST_{—} |
| | | | | | SUBUNIT PD132442: M19-L360; PD096339: I438- | PRODOM |
| | | | | | D601; PD124531: F634-Q721 | |
| | | | | | TROPOMYOSIN DM00077\|P53935\|580-755: R320- | BLAST_{—} |
| | | | | | K442 | PRODOM |
| | | | | | DM00077\|Q07283\|445-599: K327-E444 | |
| | | | | | DM00077\|P37709\|1104-1277: T330-R447 | |
| | | | | | TRICHOHYALIN DM03839\|P37709\|632-1103: | BLAST_DOMO |
| | | | | | E331-R447 | |
| | | | | | Cell attachment sequence: | MOTIFS |
| | | | | | R196-D198 | |
| 38 | 6946680CD1 | 609 | S24 S34 S64 S80 | N228 N590 | Zinc finger, C2H2 type: | HMMER_PFAM |
| | | | S89 S97 S125 S168 | | Y554-H576, Y582-H604, Y386-H408, Y330-H352, | |
| | | | S424 S568 T15 | | F442-H464, Y414-H436, F358-H380, Y302-H324, | |
| | | | T158 | | F498-H520, Y526-H548, Y470-H492 | |
| | | | | | KRAB box V14-E76 | HMMER_PFAM |
| | | | | | Zinc finger, C2H2 type, domain proteins BL00028: | BLIMPS_{—} |
| | | | | | C388-H404 | BLOCKS |
| | | | | | C2H2-type zinc finger signature PR00048: P413- | BLIMPS_{—} |
| | | | | | R426, L429-G438 | PRINTS |
| | | | | | Protein zinc finger PD01066 F16-G54 | BLIMPS_{—} |
| | | | | | | PRODOM |
| | | | | | Protein zinc finger PD00066 H376-C388 | BLIMPS_{—} |
| | | | | | | PRODOM |
| | | | | | PROTEIN ZINCFINGER METALBINDING | BLAST_{—} |
| | | | | | DNABINDING ZINC FINGER PATERNALLY | PRODOM |
| | | | | | EXPRESSED ZNFINGER PW1 PD017719: G298- | |
| | | | | | H548, F269-H520, G354-F591, G242-H492, G410- | |
| | | | | | E607 | |
| | | | | | ZINCFINGER METALBINDING DNABINDING | BLAST_{—} |
| | | | | | PROTEIN FINGER ZINC NUCLEAR REPEAT | PRODOM |
| | | | | | TRANSCRIPTION REGULATION PD001562: V14- | |
| | | | | | E76 | |
| | | | | | ZINCFINGER DNABINDING PROTEIN | BLAST_{—} |
| | | | | | METALBINDING NUCLEAR ZINC FINGER | PRODOM |
| | | | | | TRANSCRIPTION REGULATION REPEAT | |
| | | | | | PD000072: K412-C475, K524-C587, K384-C447, | |
| | | | | | K440-C503, K328-C391, R496-C559, K468-C531 | |
| | | | | | MYELOBLAST METAL-BINDING ZINC-FINGER | BLAST_{—} |
| | | | | | NUCLEAR KIAA0211 DNA-BINDING PD149061: | PRODOM |
| | | | | | E387-N589 | |
| | | | | | KRAB BOX DOMAIN DM00605\|I48208\|18-93: V14- | BLAST_DOMO |
| | | | | | R77 | |
| | | | | | DM00605\|P52738\|3-77: Q11-Q81 | |
| | | | | | DM00605\|Q05481\|10-83: G12-M79 | |
| | | | | | DM00605\|P52736\|1-72: V14-C84 | |
| | | | | | Zinc finger, C2H2 type, domain: | MOTIFS |
| | | | | | C304-H324, C332-H352, C360-H380, C388-H408, | |
| | | | | | C416-H436, C444-H464, C472-H492, C500-H520, | |
| | | | | | C528-H548, C556-H576, C584-H604 | |
| 39 | 7001142CD1 | 536 | S24 S34 S85 S108 | N341 N453 | Zinc finger, C2H2 type: | HMMER_PFAM |
| | | | S124 S198 S256 | | Y498-H520, Y330-H352, Y386-H408, Y246-H268, | |
| | | | S340 S368 T15 T97 | | Y358-H380, Y470-H492, Y414-H436, L218-H240 | |
| | | | T152 T179 T194 | | Y274-H296, Y302-H324 Y442-H464 | |
| | | | T506 | | | |
| | | | | | KRAB box: V14-K76 | HMMER_PFAM |
| | | | | | Zinc finger, C2H2 type, domain proteins BL00028: | BLIMPS_{—} |
| | | | | | C388-H404 | BLOCKS |
| | | | | | PROTEIN ZINC FINGER ZINC PD01066: F16-G54 | BLIMPS_{—} |
| | | | | | | PRODOM |
| | | | | | PROTEIN BOLA TRANSCRIPTI PD02462: T325- | BLIMPS_{—} |
| | | | | | E359, V290-E303 | PRODOM |
| | | | | | PROTEIN ZINCFINGER METALBINDING | BLAST_{—} |
| | | | | | DNABINDING ZINC FINGER PATERNALLY | PRODOM |
| | | | | | EXPRESSED ZNFINGER PW1 PD017719: G270- | |
| | | | | | H520, G242-H492, C220-H464, G326-E523, K181- | |
| | | | | | H408; PD001562: V14-K76; D000072: K440-C503, | |
| | | | | | K328-C391, K244-C307, K412-C475, R384-C447, | |
| | | | | | K356-C419, K300-C363, K272-C335, K216-C279 | |
| | | | | | ZINC FINGER PROTEIN ZINCFINGER | BLAST_{—} |
| | | | | | METALBINDING DNABINDING PUTATIVE | PRODOM |
| | | | | | REX2 TRANSCRIPTION REGULATION | |
| | | | | | PD033163: E224-K356 | |
| | | | | | KRAB BOX DOMAIN DM00605\|I48208\|18-93: V14- | BLAST_DOMO |
| | | | | | R77 | |
| | | | | | DM00605\|P52738\|3-77: Q11-S85 | |
| | | | | | DM00605\|Q05481\|10-83: L13-R77 | |
| | | | | | DM00605\|Q03923\|1-75: V14-R77 | |
| | | | | | Zinc finger, C2H2 type, domain: | MOTIFS |
| | | | | | C220-H240, C248-H268, C276-H296, C304-H324, | |
| | | | | | C332-H352, C360-H380, C388-H408, C416-H436, | |
| | | | | | C444-H464, C472-H492, C500-H520 | |
| 40 | 71158380CD1 | 643 | S295 S351 S379 | N12 | KRAB box: V4-D54 | HMMER_PFAM |
| | | | S435 T14 T36 | | | |
| | | | T142 T164 T276 | | | |
| | | | T282 T302 T508 | | | |
| | | | | | Zinc finger, C2H2 type: Y560-H582, Y225-H247, | HMMER_PFAM |
| | | | | | Y309-H331, H337-H359, Y253-H275, Y476-H498, | |
| | | | | | Y169-Q191, Y449-H470, Y131-H163, Y588-H610, | |
| | | | | | Y621-H643, H393-H415, Y281-H303, Y197-H219, | |
| | | | | | Y421-H443, Y504-H526, Y365-H387, Y532-H554 | |
| | | | | | Zinc finger, C2H2 type, domain proteins BL00028: | BLIMPS_{—} |
| | | | | | C171-H187 | BLOCKS |
| | | | | | C2H2-type zinc finger signature PR00048: P475- | BLIMPS_{—} |
| | | | | | F488, L491-G500 | PRINTS |
| | | | | | PROTEIN ZINC-FINGER META PD00066: H494- | BLIMPS_{—} |
| | | | | | C506 | PRODOM |
| | | | | | PROTEIN ZINC FINGER ZINC PD01066: L6-G44 | BLIMPS_{—} |
| | | | | | | PRODOM |
| | | | | | PROTEIN ZINCFINGER METALBINDING | BLAST_{—} |
| | | | | | DNABINDING ZINC FINGER PATERNALLY | PRODOM |
| | | | | | EXPRESSED ZNFINGER PW1 PD017719: P308- | |
| | | | | | F541, G361-F597, G221-D459, P420-H643, G389- | |
| | | | | | F630, G137-H387, G165-H415 | |
| | | | | | ZINCFINGER DNABINDING PROTEIN | BLAST_{—} |
| | | | | | METALBINDING NUCLEAR ZINC FINGER | PRODOM |
| | | | | | TRANSCRIPTION REGULATION REPEAT | |
| | | | | | PD000072: K279-C342, K419-C481, P587-H643, | |
| | | | | | K474-C537, R556-C626, K307-C370, K167-C230, | |
| | | | | | K251-C314, P336-C398, K530-C593, P196-C258, | |
| | | | | | K363-C426 | |
| | | | | | KRAB BOX DOMAIN DM00605\|P52737\|1-76: M1- | BLAST_DOMO |
| | | | | | D76 | |
| | | | | | DM00605\|I49636\|10-85: S3-D54 | |
| | | | | | ZINC FINGER, C2H2 TYPE, DOMAIN | BLAST_DOMO |
| | | | | | DM00002\|Q05481\|789-829: E412-K452, E272-Q313, | |
| | | | | | R495-E536 | |
| | | | | | Cell attachment sequence: R192-D194 | MOTIFS |
| | | | | | Zinc finger, C2H2 type, domain: C199-H219, C227- | MOTIFS |
| | | | | | H247, C255-H275, C283-H303, C311-H331, C339- | |
| | | | | | H359, C367-H387, C395-H415, C423-H443, C478- | |
| | | | | | H498, C506-H526, C534-H554, C562-H582, C590- | |
| | | | | | H610, C623-H643 | |
| 41 | 7503861CD1 | 1143 | S59 S74 S119 S145 | N72 N759 | Zinc finger, C2H2 type: | HMMER_PFAM |
| | | | S214 S251 S318 | | F1000-H1022, Y738-H755, V615-H638, Y889-H912, | |
| | | | S322 S328 S331 | | F768-H791, A1029-H1052, H703-H726, Y919-H945, | |
| | | | S334 S342 S348 | | W859-H882, Y675-H698, Y644-C666, Y587-H612 | |
| | | | S357 S383 S717 | | | |
| | | | S805 S811 S836 | | | |
| | | | S903 S935 S992 | | | |
| | | | S1011 S1081 T22 | | | |
| | | | T32 T73 T300 | | | |
| | | | T339 T379 T479 | | | |
| | | | T520 T860 T885 | | | |
| | | | T914 T955 T993 | | | |
| | | | | | Zinc finger, C2H2 type, domain proteins BL00028: | BLIMPS_{—} |
| | | | | | C891-H907 | BLOCKS |
| | | | | | PROTEIN ZINC-FINGER TALBINDING | BLIMPS_{—} |
| | | | | | DNABINDING PD00066: H634-C646 | PRODOM |
| | | | | | MYELOBLAST KIAA0211 ZINCFINGER | BLAST_{—} |
| | | | | | METALBINDING DNABINDING | PRODOM |
| | | | | | PD178887: N949-V1143; PD185235: M1-G586; | |
| | | | | | PD149061: C589-L886 | |
| | | | | | Zinc finger, C2H2 type, domain: | MOTIFS |
| | | | | | C705-H726, C770-H791, C861-H882, C891-H912, | |
| | | | | | C1031-H1052 | |
| 42 | 7758395CD1 | 1099 | S38 S94 S346 S350 | N209 N883 | Myb-like DNA-binding domain: S830-K875 | HMMER_PFAM |
| | | | S358 S438 S453 | N977 | | |
| | | | S461 S486 S494 | | | |
| | | | S572 S596 S645 | | | |
| | | | S761 S798 S898 | | | |
| | | | S910 S923 S962 | | | |
| | | | S979 S1002 S1012 | | | |
| | | | S1019 S1059 T454 | | | |
| | | | T499 T513 T568 | | | |
| | | | T573 T687 T786 | | | |
| | | | T828 T873 T887 | | | |
| | | | T893 T1009 T1025 | | | |
| | | | Y46 Y654 | | | |
| | | | | | Zinc finger, C2H2 type, domain: | MOTIFS |
| | | | | | C1040-H1060 | |
| 43 | 71039312CD1 | 1006 | S52 S67 S79 S130 | N329 N735 | Zinc finger, C2H2 type: F668-H692, Y551-H575, | HMMER_PFAM |
| | | | S156 S163 S173 | | F610-H634, Y490-H515 | |
| | | | S205 S213 S231 | | | |
| | | | S237 S258 S310 | | | |
| | | | S337 S374 S433 | | | |
| | | | S504 S530 S564 | | | |
| | | | S687 S720 S721 | | | |
| | | | S732 S861 S899 | | | |
| | | | T181 T196 T225 | | | |
| | | | T259 T332 T367 | | | |
| | | | T445 T452 T503 | | | |
| | | | T579 T620 T715 | | | |
| | | | Y346 Y656 | | | |
| | | | | | FINGER ZINC DM04988 | BLAST_DOMO |
| | | | | | \|JH0797\|457-514: H571-I627 | |
| | | | | | \|JH0797\|396-455: I509-V568 | |
| | | | | | \|JH0797\|516-572: E628-M685 | |
| | | | | | ATP/GTP-binding site motif A (P-loop): G541-S548 | MOTIFS |
| | | | | | Zinc finger, C2H2 type, domain: C553-H575, C612- | MOTIFS |
| | | | | | H634, C670-H692 | |
| 44 | 7291318CD1 | 768 | S15 S27 S239 S313 | N19 | signal_cleavage: M1-A34 | SPSCAN |
| | | | S410 S416 S428 | | | |
| | | | S446 S464 S539 | | | |
| | | | S607 T75 T154 | | | |
| | | | T219 T222 T285 | | | |
| | | | T338 T403 T657 | | | |
| | | | Y417 | | | |
| | | | | | Zinc finger, C2H2 type: Y359-H381, F480-H502, F58- | HMMER_PFAM |
| | | | | | C81, F625-G648, Y564-Q591, L236-H258, Y536- | |
| | | | | | H558, H597-H619, F264-H286, Y417-H439, F508- | |
| | | | | | H530, Y387-H411, Y454-H478 | |
| | | | | | Zinc finger, C2H2 type, domain proteins BL00028: | BLIMPS_{—} |
| | | | | | C482-H498 | BLOCKS |
| | | | | | Cytidine and deoxycytidylate deaminases zinc-binding | BLIMPS_{—} |
| | | | | | regions BL00903: A476-C485 | BLOCKS |
| | | | | | PROTEIN ZINC-FINGER META PD00066: H254- | BLIMPS_{—} |
| | | | | | C266 | PRODOM |
| | | | | | Zinc finger, C2H2 type, domain: C238-H258, C266- | MOTIFS |
| | | | | | H286, C361-H381, C389-H411, C419-H439, C456- | |
| | | | | | H478, C482-H502, C510-H530, C538-H558, C599- | |
| | | | | | H619 | |
| 45 | 2638619CD1 | 561 | S3 S27 S32 S71 | N69 N177 | ELM2 domain: K211-P272 | HMMER_PFAM |
| | | | S119 S148 S157 | | | |
| | | | S158 S197 S249 | | | |
| | | | S319 S349 S363 | | | |
| | | | S395 S459 S483 | | | |
| | | | S501 S521 T167 | | | |
| | | | T199 T386 T476 | | | |
| | | | Y236 Y259 Y288 | | | |
| | | | Y356 Y368 | | | |
| | | | | | Myb-like DNA-binding domain: L315-K361 | HMMER_PFAM |
| | | | | | ER1 PD126939: E42-Q285 | BLAST_{—} |
| | | | | | | PRODOM |
| | | | | | PROTEIN METASTASIS-ASSOCIATED MTA1 | BLAST_{—} |
| | | | | | SIMILAR MTA1 T27C4.4 KIAA0458 C04A2.2 | PRODOM |
| | | | | | CHROMOSOME II PD011563: A286-R377 | |
| 46 | 2810014CD1 | 123 | S23 S53 S63 S70 | N80 N113 | signal_cleavage: M1-A29 | SPSCAN |
| | | | S77 S97 T57 T84 | | | |
| 47 | 3457155CD1 | 1236 | S10 S18 S45 S97 | N299 N704 | LYASE PROTEIN PHYCOBILIS PD01642 S874- | BLIMPS_{—} |
| | | | S186 S318 S333 | | E902 | PRODOM |
| | | | S337 S342 S348 | | | |
| | | | S364 S365 S449 | | | |
| | | | S470 S485 S728 | | | |
| | | | S846 S872 S892 | | | |
| | | | S917 S929 S1019 | | | |
| | | | S1096 S1166 S1209 | | | |
| | | | T16 T86 T135 | | | |
| | | | T494 T581 T1031 | | | |
| | | | T1079 T1159 | | | |
| | | | T1190 Y566 Y1095 | | | |
| | | | | | TB-Binding Protein TIP120 PD044220 A4-D1223 | BLAST- |
| | | | | | | PRODOM |
| | | | | | Leucine zipper pattern: L155-L176 | MOTIFS |
| 48 | 7435171CD1 | 357 | S32 S135 S147 | N294 | signal_cleavage: M1-A67 | SPSCAN |
| | | | S171 S180 S185 | | | |
| | | | S244 S253 S255 | | | |
| | | | S314 T149 T230 | | | |
| | | | | | Homeobox domain: K228-R284 | HMMER_PFAM |
| | | | | | Homeobox′ domain proteins BL00027: L242-R284 | BLIMPS_{—} |
| | | | | | | BLOCKS |
| | | | | | Homeobox′ antennapedia-type protein BL00032: | BLIMPS_{—} |
| | | | | | A198-E220, R231-T269, Q270-A287 | BLOCKS |
| | | | | | Homeobox′ domain signature and profile: Q241-V304 | PROFILESCAN |
| | | | | | Homeobox signature PR00024: K249-L260, L264- | BLIMPS_{—} |
| | | | | | W274, W274-K283 | PRINTS |
| | | | | | PROTEIN HOMEOBOX DNA-BINDING | BLAST_{—} |
| | | | | | NUCLEAR NKX5.1 DEVELOPMENTAL | PRODOM |
| | | | | | HOMEODOMAIN NKX51 | |
| | | | | | PD034587: F83-R226 | |
| | | | | | PD019212: L286-V357 | |
| | | | | | PROTEIN HOMEOBOX DN-BINDING NUCLEAR | BLAST_{—} |
| | | | | | DEVELOPMENTAL TRANSCRIPTION | PRODOM |
| | | | | | REGULATION FACTOR HOMEODOMAIN | |
| | | | | | METAL-BINDING PD000010: R226-Q285 | |
| | | | | | HOMEOBOX DM00009 | BLAST_DOMO |
| | | | | | \|P42581\|325-388: P223-A287 | |
| | | | | | \|I48690\|325-388: P223-A287 | |
| | | | | | \|A47234\|192-259: R226-A287 | |
| | | | | | \|B41224\|153-215: R226-Q285 | |
| | | | | | Homeobox′ domain signature: L260-K283 | MOTIFS |
| 49 | 7499936CD1 | 168 | S20 T151 | | signal_cleavage: M1-A43 | SPSCAN |
| | | | | | Ligand-binding domain of nuclear hormone: G10- | HMMER_PFAM |
| | | | | | L161 | |
| | | | | | Retinoic acid receptor signature PR00545: N12-G29, | BLIMPS_{—} |
| | | | | | F52-E72, P92-Y109, K113-R132, E140-E159 | PRINTS |
| | | | | | RECEPTOR PROTEIN NUCLEAR | BLAST_{—} |
| | | | | | TRANSCRIPTION REGULATION DNA-BINDING | PRODOM |
| | | | | | ZINC FINGER HORMONE FAMILY MULTIGENE | |
| | | | | | PD000112: L7-I134 | |
| | | | | | RECEPTOR TRANSCRIPTION REGULATION | BLAST_{—} |
| | | | | | DNA-BINDING NUCLEAR PROTEIN ZINC | PRODOM |
| | | | | | FINGER RETINOIC ACID MULTIGENE | |
| | | | | | PD149760: G135-H165 | |
| | | | | | NUCLEAR HORMONES RECEPTORS DNA- | BLAST_DOMO |
| | | | | | BINDING REGION DM00047 | |
| | | | | | \|Q05343\|130-391: L7-A93 | |
| | | | | | \|P28700\|130-391: L7-A93 | |
| | | | | | \|P19793\|125-386: L7-A93 | |
| | | | | | \|C41727\|130-391: L7-A93 | |
| 50 | 7504125CD1 | 142 | S139 T27 | N50 | signal_cleavage: M1-D17 | SPSCAN |
| | | | | | Ets-domain: A4-K69 | HMMER_PFAM |
| | | | | | ETS domain signature PR00454: I5-Q18, N29-L47, | BLIMPS_{—} |
| | | | | | R48-Y66 | PRINTS |
| | | | | | Ets-domain proteins BL00345: M1-K19, K34-S84 | BLIMPS_{—} |
| | | | | | | BLOCKS |
| | | | | | Ets-domain signatures and profile: S3-L35, G31- | PROFILESCAN |
| | | | | | T113 | |
| | | | | | ETS DOMAIN PROTEIN NUCLEAR DNA- | BLAST_{—} |
| | | | | | BINDING ACCESSORY FACTOR | PRODOM |
| | | | | | TRANSCRIPTION SERUM RESPONSE ELK4 | |
| | | | | | PD008319: Y65-S142 | |
| | | | | | PROTEIN DNA-BINDING NUCLEAR | BLAST_{—} |
| | | | | | TRANSCRIPTION FACTOR REGULATION ETS | PRODOM |
| | | | | | PROTO-ONCOGENE ACTIVATOR | |
| | | | | | ALTERNATIVE PD000803: I5-K69 | |
| | | | | | ETS-DOMAIN DM02126\|P41970\|98-406: Y65-S142 | BLAST_DOMO |
| | | | | | ETS-DOMAIN DM00281 | BLAST_DOMO |
| | | | | | \|P41970\|1-96: M1-K69 | |
| | | | | | \|I48680\|1-96: M1-K69 | |
| | | | | | ETS-DOMAIN DM02126\|I48680\|98-409: Y65-K141 | BLAST_DOMO |
| 51 | 7505742CD1 | 477 | S91 S216 S248 | N220 N316 | Ets-domain signature 1: L7-L15 | MOTIFS |
| | | | S265 S423 S443 | N341 | Ets-domain signature 2: K51-Y66 | |
| | | | T191 T258 T266 | | | |
| | | | T267 T286 | | | |
| | | | | | Fork head domain: K169-R264 | HMMER_PFAM |
| | | | | | Fork head domain signature PR00053: K169-I182, | BLIMPS_{—} |
| | | | | | L190-R207, W213-V230 | PRINTS |
| | | | | | Fork head domain proteins BL00657: K169-K210, | BLIMPS_{—} |
| | | | | | Q214-G256 | BLOCKS |
| | | | | | Fork head domain signatures and profile: L101-G194 | PROFILESCAN |
| | | | | | TRANSCRIPTION FACTOR DNA-BINDING | BLAST_{—} |
| | | | | | NUCLEAR PROTEIN BF1 BRAIN REGULATION | PRODOM |
| | | | | | DEVELOPMENTAL BF1 PD009393: I254-P412 | |
| | | | | | PROTEIN TRANSCRIPTION FACTOR NUCLEAR | BLAST_{—} |
| | | | | | DNA-BINDING REGULATION FORK HEAD | PRODOM |
| | | | | | FORKHEAD DOMAIN PD000425: K169-R264 | |
| | | | | | TRANSCRIPTION FACTOR BRAIN | BLAST_{—} |
| | | | | | REGULATION DNA-BINDING NUCLEAR | PRODOM |
| | | | | | PROTEIN DEVELOPMENTAL BF1 BF1 | |
| | | | | | PD012927: S413-H477 | |
| | | | | | TRANSCRIPTION FACTOR BF1 BRAIN 1 BF1 | BLAST_{—} |
| | | | | | HFK1 REGULATION DNA-BINDING NUCLEAR | PRODOM |
| | | | | | PROTEIN DEVELOPMENTAL PD049691: G86- | |
| | | | | | D131 | |
| | | | | | FORK HEAD DNA-BINDING DOMAIN DM00381 | BLAST_DOMO |
| | | | | | \|P55315\|58-332: P58-L333 | |
| | | | | | \|A47446\|44-314: H48-L333, H37-K152 | |
| | | | | | \|P32031\|72-344: P122-P326 | |
| | | | | | \|P32030\|22-301: E142-A278, H50-P58, H52-E94, | |
| | | | | | P45-H54 | |
| | | | | | Fork head domain signature 1: K169-I182 | MOTIFS |
| | | | | | Fork head domain signature 2: W213-H219 | |
| 52 | 7505757CD1 | 1274 | S10 S18 S83 S135 | N337 N742 | LYASE PROTEIN PHYCOBILIS PD01642: S912- | BLIMPS_{—} |
| | | | S224 S356 S371 | | E940 | PRODOM |
| | | | S375 S380 S386 | | | |
| | | | S402 S403 S487 | | | |
| | | | S508 S523 S766 | | | |
| | | | S884 S910 S930 | | | |
| | | | S955 S967 S1057 | | | |
| | | | S1134 S1204 S1247 | | | |
| | | | T16 T124 T173 | | | |
| | | | T532 T619 T1069 | | | |
| | | | T1117 T1197 | | | |
| | | | T1228 Y604 Y1133 | | | |
| | | | | | PUTATIVE TB-BINDING PROTEIN TIP 120 | BLAST_{—} |
| | | | | | PD044220: R61-D1261 | PRODOM |
| | | | | | Leucine zipper pattern: L193-L214 | MOTIFS |
| 53 | 7504126CD1 | 91 | S24 S89 T2 T55 | | signal_cleavage: M1-A45 | SPSCAN |
| | | | | | RIBOSOMAL PROTEIN 40S S5 5S PROBABLE | BLAST_{—} |
| | | | | | PD004090: M1-Q36 | PRODOM |
| | | | | | RIBOSOMAL PROTEIN S7 DM00334 | BLAST_DOMO |
| | | | | | \|P49041\|182-209: Q36-R91 | |
| | | | | | \|P26783\|96-223: Q36-R91 | |
| 54 | 7504099CD1 | 311 | S169 S196 S202 | | HPBRII4 MRNA | BLAST_{—} |
| | | | S220 S231 S248 | | PD112364: M1-P70 | PRODOM |
| | | | S266 T163 Y281 | | PD066177: V116-R165 | |
| | | | Y308 | | PD029583: T166-Q233 | |
| | | | | | PD175646: D234-Y281 | |
| | | | | | HPBRII; DM05499\|S57447\| | BLAST_DOMO |
| | | | | | 356-450: H115-A210 | |
| | | | | | 251-354: P56-P114, P57-P148, G67-G158 | |
| | | | | | PROLINE-RICH PROTEIN DM03894\|P05142\|1- | BLAST_DOMO |
| | | | | | 134: P57-P147, V36-G158, P56-P114 | |
| | | | | | FIBRILLAR COLLAGEN CARBOXYL- | BLAST_DOMO |
| | | | | | TERMINAL DM00042\|A41132\|43-133: P56-P124, | |
| | | | | | P58-P142, P56-V116 | |
| | | | | | Cell attachment sequence: R151-D153 | MOTIFS |
| 55 | 7505733CD1 | 110 | S90 S100 | | signal_cleavage: M1-L63 | SPSCAN |
| | | | | | Ribosomal protein S24e signature: S21-I75 | PROFILESCAN |
| | | | | | PROTEASE ORF DERIVED FROM INTEGRASE | BLAST_{—} |
| | | | | | CODING REGION REGIONS D1 LEADER | PRODOM |
| | | | | | PD152194: D20-Q110 | |
| 56 | 7959829CD1 | 176 | T6 T11 T51 T88 | N4 N9 | SYNTHASE I PSEUDOURIDYLATE PD02906: | BLIMPS_{—} |
| | | | T117 | N86 | C114-Q126, L130-L164, Y71-E83 | PRODOM |
| | | | | | SYNTHASE; PSEUDOURTOYLATE; TRNA; | BLAST_DOMO |
| | | | | | PSEUDOURIDINE; DM02282 | |
| | | | | | \|Q09524\|29-297: R62-N165 | |
| | | | | | \|P31115\|91-343: R63-R166 | |
| 57 | 7502168CD1 | 532 | S6 S19 S37 S90 | N519 | chromo′ (CHRromatin Organization Modifier): E9- | HMMER_PFAM |
| | | | S118 S119 S120 | | I49 | |
| | | | S126 S360 S409 | | | |
| | | | S411 S426 S438 | | | |
| | | | S459 S465 S467 | | | |
| | | | S469 S498 T84 | | | |
| | | | T176 T188 T281 | | | |
| | | | T294 T408 T454 | | | |
| | | | T489 | | | |
| | | | | | Chromo domain proteins BL00598: E28-I49 | BLIMPS_{—} |
| | | | | | | BLOCKS |
| | | | | | Chromo domain signature and profile: I17-Q68 | PROFILESCAN |
| | | | | | Chromodomain signature PR00504: E9-I17, L22- | BLIMPS_{—} |
| | | | | | W36, S37-I49 | PRINTS |
| | | | | | MODIFIER 3 PROTEIN M33 NUCLEAR | BLAST_{—} |
| | | | | | TRANSCRIPTION REGULATION REPRESSOR | PRODOM |
| | | | | | PD138310: K131-T506 | |
| | | | | | CHROMO DOMAIN DM00963 | BLAST_DOMO |
| | | | | | \|P30658\|1-190: M1-R190 | |
| | | | | | \|P34618\|1-189: G8-I160 | |
| | | | | | \|P05205\|13-184: E9-R132 | |
| | | | | | \|P45973\|9-158: S5-K96 | |
| | | | | | Chromo domain signature: Y29-I49 | MOTIFS |
| 58 | 7503888CD1 | 1492 | S2 S78 S111 S446 | N248 N563 | SNF2 and others N-terminal domain: Y757-F1052 | HMMER_PFAM |
| | | | S655 S660 S662 | N1302 | | |
| | | | S699 S1022 S1058 | | | |
| | | | S1227 S1335 S1366 | | | |
| | | | S1415 S1420 S1431 | | | |
| | | | S1472 S1476 S1487 | | | |
| | | | S1489 S442 S624 | | | |
| | | | S833 S850 S1079 | | | |
| | | | S1155 S1254 S1255 | | | |
| | | | S1272 S1282 S1322 | | | |
| | | | S1404 S1437 S1262 | | | |
| | | | S1462 S937 T428 | | | |
| | | | T511 T629 T858 | | | |
| | | | T1110 T1130 | | | |
| | | | T1141 T1203 | | | |
| | | | T1241 T11 T308 | | | |
| | | | T453 T494 T739 | | | |
| | | | T1129 T1229 | | | |
| | | | T1304 Y90 Y718 | | | |
| | | | Y1224 | | | |
| | | | | | Bromodomain: M1307-V1397, K1140-S1155 | HMMER_PFAM |
| | | | | | Helicase conserved C-terminal domain: T1110-G1194 | HMMER_PFAM |
| | | | | | Bromodomain proteins BL00633: L918-P930, P1340- | BLIMPS_{—} |
| | | | | | Y1364, D1373-N1385 | BLOCKS |
| | | | | | Bromodomain signature PR00503: Q1325-E1338, | BLIMPS_{—} |
| | | | | | L1339-I1355, I1355-D1373 | PRINTS |
| | | | | | Bromodomain signature and profile: P1334-S1404 | PROFILESCAN |
| | | | | | I ATP-BINDING NUCLEOSIDE PD02191: Y877- | BLIMPS_{—} |
| | | | | | C891, N898-N926, K997-Y1008, V1171-Q1196 | PRODOM |
| | | | | | PROTEIN BROMODOMAIN HELICASE | BLAST_{—} |
| | | | | | NUCLEAR ATP-BINDING TRANSCRIPTION | PRODOM |
| | | | | | REGULATION ACTIVATOR BRAHMA | |
| | | | | | POSSIBLE PD007692: E365-K572 | |
| | | | | | PROTEIN HELICASE ATP-BINDING NUCLEAR | BLAST_{—} |
| | | | | | DNA-BINDING ZINC FINGER DNA | PRODOM |
| | | | | | TRANSCRIPTION REPAIR I PD000441: L870- | |
| | | | | | L1035, I932-M1050, N771-E821, Y757-I793, G390- | |
| | | | | | E449, L456-I479, Q460-A509 | |
| | | | | | PROTEIN POSSIBLE GLOBAL TRANSCRIPTION | BLAST_{—} |
| | | | | | ACTIVATOR REGULATION NUCLEAR | PRODOM |
| | | | | | BROMODOMAIN ATP-BINDING HELICASE | |
| | | | | | PD017589: G594-K687 | |
| | | | | | PROTEIN POSSIBLE GLOBAL TRANSCRIPTION | BLAST_{—} |
| | | | | | ACTIVATOR REGULATION NUCLEAR | PRODOM |
| | | | | | BROMODOMAIN ATP-BINDING HELICASE | |
| | | | | | PD151443: E286-V364 | |
| | | | | | BROMODOMAIN DM02887 | BLAST_DOMO |
| | | | | | \|P51532\|177-770: L177-N771 | |
| | | | | | \|S45252\|177-770: L177-N771 | |
| | | | | | \|S39059\|176-768: L177-N770 | |
| | | | | | ATP NP_BIND DM00266\|S45252\|772-1200: N772- | BLAST_DOMO |
| | | | | | V1199 | |
| | | | | | Bromodomain signature: S1327-F1384 | MOTIFS |
| | | | | | Leucine zipper pattern: L907-L928 | MOTIFS |

TABLE 4

| Polynucleotide | |
|---|---|
| SEQ ID NO:/ | |
| Incyte ID/Sequence | |
| Length | Sequence Fragments |
| 59/7503848CB1/ | 1-236, 1-522, 4-541, 21-268, 21-662, 29-630, 87-618, 131-5006, 147-537, 469-723, 555-861, 555-1022, 668-769, |
| 5007 | 668-770, 668-859, 668-1053, 688-750, 688-765, 688-766, 688-832, 688-1041, 693-832, 726-988, 727-840, 728-840, |
| | 742-840, 749-846, 757-846, 786-832, 791-850, 796-850, 800-840, 818-846, 868-1008, 871-1410, 874-958, 874-980, |
| | 874-982, 874-996, 874-1062, 875-982, 881-932, 881-1069, 881-1248, 886-1053, 902-978, 902-996, 902-1053, 909-1062, |
| | 910-1056, 942-1041, 942-1181, 955-1041, 995-1032, 995-1062, 1004-1296, 1013-1098, 1013-1107, 1013-1112, |
| | 1013-1133, 1013-1160, 1013-1177, 1013-1189, 1013-1190, 1013-1266, 1014-1053, 1014-1062, 1052-1151, |
| | 1052-1190, 1052-1266, 1059-1153, 1070-1290, 1080-1321, 1096-1127, 1096-1240, 1096-1266, 1096-1446, 1097-1248, |
| | 1101-1266, 1137-1240, 1172-1240, 1182-1721, 1192-1241, 1253-1283, 1257-1776, 1275-1325, 1346-1458, |
| | 1354-2012, 1382-1450, 1386-1974, 1449-1708, 1450-1968, 1453-1783, 1479-1770, 1556-1650, 1597-2185, 1604-1766, |
| | 1654-1912, 1658-1688, 1660-1974, 1683-1988, 1683-2172, 1683-2180, 1683-2212, 1683-2227, 1807-2276, |
| | 1890-2569, 1955-2210, 1984-2582, 2021-2266, 2021-2513, 2035-2594, 2070-2112, 2076-2112, |
| | 2076-2578, 2107-2577, 2113-2372, 2156-2677, 2198-2458, 2210-2492, 2212-2478, 2231-2490, 2253-2569, 2260-2540, |
| | 2306-2593, 2306-2599, 2344-2755, 2381-2917, 2383-2434, 2387-2558, 2387-2637, 2388-2430, 2394-2430, |
| | 2432-2568, 2442-2559, 2449-2557, 2449-2628, 2460-2637, 2462-2637, 2476-2611, 2476-2701, 2477-2628, 2479-2654, |
| | 2517-2611, 2551-2765, 2564-2619, 2586-2642, 2593-2628, 2623-2933, 2684-2979, 2686-2932, 2686-2979, |
| | 2686-3144, 2686-3342, 2686-3378, 2715-2979, 2759-3342, 2895-3128, 2897-3153, 2906-3152, 2921-3192, 2921-3384, |
| | 2952-3529, 2976-3387, 2977-3137, 2977-3216, 2980-3411, 2993-3287, 2999-3263, 3004-3303, 3020-3283, |
| | 3042-3287, 3059-3936, 3091-3595, 3101-3377, 3120-3337, 3161-3971, 3167-3492, 3192-3823, 3192-3864, 3196-3959, |
| | 3201-3486, 3235-3842, 3237-3797, 3240-3533, 3248-3926, 3268-3691, 3270-3842, 3273-3495, 3273-3700, |
| | 3304-3573, 3307-3556, 3308-3580, 3340-3607, 3340-3619, 3413-3854, 3415-3544, 3421-4049, 3427-3950, 3439-3839, |
| | 3447-3744, 3453-3730, 3495-3741, 3496-3928, 3504-3689, 3504-4027, 3527-3738, 3536-3647, 3538-3810, |
| | 3539-4033, 3543-3778, 3550-4040, 3810-3929, 3818-4133, 3837-4109, 3837-4118, |
| | 3947-4019, 3947-4023, 3947-4024, 3947-4026, 3947-4027, 3947-4028, 3947-4032, 3947-4051, 3947-4094, 3947-4101, |
| | 3947-4103, 3949-4040, 3954-4036, 3955-4094, 3994-4221, 4003-4238, 4011-4294, 4012-4122, 4015-4227, |
| | 4028-4297, 4032-4240, 4039-4343, 4068-4654, 4070-4656, 4078-4622, 4082-4372, 4091-4228, 4102-4652, 4128-4262, |
| | 4128-4481, 4128-4638, 4132-4244, 4147-4423, 4151-4410, 4151-4462, 4152-4471, 4194-4594, 4205-4594, |
| | 4219-4594, 4246-4465, 4246-4527, 4246-4556, 4246-4651, 4263-4658, 4282-4559, 4282-4633, 4282-4651, 4299-4594, |
| | 4304-4585, 4349-4502, 4356-4633, 4360-4594, 4377-4594, 4378-4594, 4384-4594, 4390-4524, 4399-4594, |
| | 4404-4537, 4404-4538, 4404-4867, 4412-5006, 4422-4655, 4424-4656, 4428-4594, 4441-4594, 4449-4594, 4461-4594, |
| | 4469-4594, 4520-5007, 4620-4881 |
| 60/2608080CB1/ | 1-592, 26-591, 26-592, 41-542, 100-592, 395-787, 395-797, 395-799, 395-800, 395-801, 402-627, 574-798, 574-799, |
| 3118 | 574-800, 579-798, 595-787, 602-1271, 826-1464, 842-1463, 853-1455, 1041-1090, 1051-1172, 1057-1088, |
| | 1059-1175, 1173-1573, 1173-1819, 1210-1267, 1210-1286, 1210-1295, 1210-1373, 1210-1388, 1210-1405, 1210-1424, |
| | 1210-1429, 1210-1435, 1210-1511, 1210-1514, 1210-1592, 1210-1595, 1210-1679, 1213-1334, 1216-1250, |
| | 1216-1302, 1216-1343, 1219-1334, 1225-1256, 1227-1344, 1231-1595, 1234-1344, 1234-1535, 1296-1709, 1297-1429, |
| | 1304-1429, 1315-1556, 1315-1847, 1321-1598, 1321-1758, 1366-1758, 1368-1427, 1378-1610, 1378-1618, |
| | 1378-1741, 1378-1931, 1399-1758, 1464-1595, 1465-1535, 1465-1865, 1471-1595, 1482-1725, 1482-2016, 1492-1766, |
| | 1492-1926, 1535-1926, 1540-1926, 1541-1590, 1542-1596, 1542-1676, 1543-1590, 1545-1594, 1546-1590, |
| | 1546-1771, 1546-1775, 1546-1906, 1546-2094, 1548-1679, 1549-1680, 1549-1841, 1580-1841, 1605-2363, 1616-2363, |
| | 1620-1679, 1626-1679, 1626-1760, 1632-1758, 1636-1722, 1636-1764, 1636-1766, 1639-1678, 1639-1758, |
| | 1639-1762, 1640-2039, 1640-2363, 1647-1946, 1660-1892, 1660-2094, 1713-1754, |
| | 1714-1758, 1714-1906, 1714-1928, 1714-2075, 1714-2099, 1800-1926, 1801-1928, 1801-1932, 1801-2093, 1801-2099, |
| | 1806-1926, 1816-2094, 1819-2094, 1822-1932, 1822-2123, 1828-2060, 1828-2094, 1876-2094, 1877-1926, |
| | 1882-2101, 1917-2094, 1965-2161, 1965-2570, 1969-2090, 1969-2100, 1973-2589, 1984-2094, 1987-2094, 1992-2099, |
| | 2045-2288, 2046-2099, 2053-2123, 2201-3044, 2517-3031, 2549-2911, 2646-3109, 2675-3109, 2766-3108, |
| | 2947-3118 |
| 61/7503402CB1/ | 1-174, 2-2899, 6-480, 53-629, 53-759, 58-705, 196-671, 206-565, 237-747, 300-328, 306-927, 325-881, 357-956, |
| 2909 | 361-507, 378-406, 394-1048, 408-1001, 428-596, 441-1096, 450-1071, 495-1115, 527-1064, 531-1192, 573-1252, |
| | 607-1139, 609-850, 609-1145, 719-1351, 779-1241, 791-1381, 812-1489, 825-853, 829-853, 845-1435, 851-1114, |
| | 851-1296, 860-1103, 864-1209, 883-1568, 901-1438, 909-1165, 913-1601, 915-939, 915-943, 918-1144, 920-1564, |
| | 922-1377, 945-1637, 951-1635, 1009-1781, 1017-1470, 1026-1605, 1035-1635, 1061-1340, 1062-1674, 1077-1669, |
| | 1090-1766, 1121-1828, 1131-1714, 1141-1435, 1149-1387, 1152-1709, 1192-1693, 1194-1838, 1199-1487, 1213-1757, |
| | 1234-1841, 1257-1777, 1260-1703, 1268-1870, 1296-1774, 1310-1521, 1310-1546, 1310-1776, 1310-1965, |
| | 1310-2107, 1326-2085, 1361-2033, 1396-1953, 1406-1758, 1421-1715, 1435-1980, 1439-1709, 1439-2057, 1453-1720, |
| | 1468-2101, 1479-2025, 1479-2067, 1483-1743, 1487-1776, 1488-2101, 1513-1902, 1525-2202, 1540-2188, |
| | 1544-2219, 1556-2237, 1575-2241, 1580-1847, 1593-2239, 1621-1934, 1645-2229, 1727-2235, 1728-2235, 1778-2235, |
| | 1790-1860, 1795-2057, 1843-2164, 1874-2245, 1886-2000, 1913-2529, 1925-2334, 1942-2334, |
| | 1946-2334, 1951-2309, 1952-2179, 1959-2256, 2024-2305, 2055-2249, 2071-2331, 2092-2243, 2092-2401, 2099-2636, |
| | 2131-2545, 2148-2363, 2148-2390, 2169-2443, 2228-2658, 2266-2542, 2292-2625, 2305-2485, 2338-2789, |
| | 2349-2791, 2405-2645, 2426-2673, 2426-2884, 2426-2909, 2437-2569, 2493-2791, 2495-2794, 2500-2792, 2500-2793, |
| | 2549-2794, 2724-2794 |
| 62/7503517CB1/ | 1-261, 1-372, 1-502, 47-627, 112-353, 119-361, 119-366, 121-385, 122-431, 122-445, 124-353, 124-443, 127-774, |
| 1613 | 132-692, 137-363, 139-441, 144-477, 144-722, 151-452, 152-281, 152-373, 172-437, 458-727, 516-778, 517-1604, |
| | 520-788, 546-627, 546-704, 662-1295, 662-1330, 712-1231, 791-1275, 793-1026, 797-1311, 798-1336, 816-1079, |
| | 840-1438, 843-1101, 873-1171, 876-1534, 898-1084, 915-1211, 949-1104, 956-1298, 957-1206, 960-1594, 971-1572, |
| | 974-1219, 980-1260, 982-1602, 1004-1582, 1006-1105, 1006-1179, 1020-1296, 1038-1163, 1068-1555, 1091-1603, |
| | 1098-1552, 1099-1604, 1103-1613, 1110-1613, 1126-1599, 1132-1267, 1143-1603, 1152-1385, 1162-1589, |
| | 1169-1608, 1200-1603, 1200-1604, 1221-1482, 1244-1488, 1264-1596, 1285-1508, 1285-1509, 1285-1520, 1285-1603, |
| | 1316-1561, 1317-1603, 1324-1602, 1329-1607, 1335-1602, 1336-1571, 1357-1603, 1428-1613, 1440-1612, |
| | 1537-1613 |
| 63/7500014CB1/ | 1-194, 1-1006, 14-217, 22-217, 23-567, 23-581, 23-651, 23-813, 23-874, 23-962, 23-990, 23-991, 34-210, 39-993, |
| 1022 | 99-990, 210-990, 211-973, 214-990, 215-992, 222-780, 225-792, 225-850, 226-333, 226-471, 228-990, 229-993, 229-1020, |
| | 238-475, 246-954, 251-858, 253-928, 256-518, 258-694, 258-939, 261-993, 262-1012, 268-942, 270-510, 270-1008, |
| | 276-537, 276-733, 276-870, 288-878, 290-556, 293-884, 312-880, 313-551, 314-993, 317-569, 319-555, 319-588, |
| | 322-575, 323-561, 323-587, 323-993, 327-819, 329-834, 329-987, 341-927, 341-1019, 344-954, 345-790, 346-541, |
| | 346-912, 348-993, 350-992, 351-1022, 352-546, 352-627, 356-992, 359-616, 365-948, 366-1021, 372-614, 373-626, |
| | 377-782, 377-881, 377-897, 394-631, 395-987, 401-925, 406-1000, 415-682, 419-993, 425-728, 425-992, 426-687, |
| | 426-720, 431-706, 440-942, 448-700, 448-993, 450-771, 454-944, 466-1000, 470-709, 472-684, 493-839, 506-776, |
| | 526-956, 555-781, 566-862, 569-840, 650-1020, 695-931, 696-890 |
| 64/7501365CB1/ | 1-478, 1-1592, 20-289, 28-437, 29-288, 50-289, 57-658, 184-465, 282-569, 282-687, 291-609, 291-897, 298-756, |
| 1816 | 317-682, 331-762, 345-953, 417-871, 462-871, 503-871, 507-871, 519-701, 562-1090, 592-753, 629-896, 670-846, |
| | 700-923, 713-1248, 723-1236, 805-1115, 830-1089, 888-1151, 889-1135, 1126-1587, 1150-1256, 1151-1283, 1152-1360, |
| | 1152-1816, 1273-1314, 1337-1443, 1459-1514, 1572-1612, 1623-1646, 1623-1730, 1734-1789 |
| 65/7503540CB1/ | 1-591, 1-596, 1-651, 1-671, 1-690, 1-712, 3-638, 3-741, 85-631, 189-632, 221-631, 222-5935, 266-670, 356-676, |
| 5955 | 625-900, 886-1112, 913-935, 999-1671, 1361-1630, 1361-1632, 1361-1879, 1438-2012, 1538-1749, 1613-1856, |
| | 1653-1903, 1656-1901, 1656-1927, 1770-2076, 1831-2305, 2024-2599, 2173-2689, 2174-2689, 2252-2828, 2261-2694, |
| | 2378-3163, 2404-3012, 2425-2985, 2426-2974, 2447-2694, 2690-2875, 2690-2927, 2802-3574, 2812-3321, |
| | 2823-3100, 3003-3276, 3203-3659, 3410-3748, 3487-3747, 3495-3804, 3501-3805, 3711-3940, 3758-4044, 4067-4484, |
| | 4147-4484, 4421-4547, 4574-5120, 4629-4890, 4629-5139, 4684-4928, 4691-4907, 4710-4963, 4710-4965, |
| | 4710-4998, 4710-5005, 4710-5172, 4726-4967, 4742-5015, 4766-5336, 4820-5086, 4822-5109, 4854-5100, 4862-5135, |
| | 4871-5123, 4907-5351, 4907-5360, 4968-5493, 4973-5372, 5020-5710, 5059-5283, 5135-5389, 5137-5374, |
| | 5141-5498, 5141-5634, 5144-5412, 5154-5418, 5154-5745, 5156-5431, 5164-5506, 5174-5598, 5175-5627, 5181-5827, |
| | 5210-5429, 5216-5866, 5218-5454, 5236-5556, 5240-5479, 5240-5632, 5253-5461, 5255-5509, 5261-5559, |
| | 5269-5470, 5272-5483, 5272-5517, 5272-5561, 5286-5592, 5286-5948, 5286-5955, 5311-5569, 5333-5504, |
| | 5338-5652, 5342-5568, 5352-5612, 5352-5781, 5374-5779, 5386-5482, 5388-5690, 5405-5664, 5425-5667, 5445-5938, |
| | 5451-5948, 5458-5791, 5458-5953, 5459-5930, 5460-5955, 5462-5780, 5471-5726, 5477-5656, 5480-5690, |
| | 5481-5929, 5481-5955, 5482-5937, 5488-5745, 5489-5931, 5492-5955, 5503-5740, 5510-5868, 5516-5937, 5519-5937, |
| | 5524-5938, 5529-5937, 5532-5940, 5555-5836, 5556-5937, 5561-5948, 5565-5865, 5584-5851, 5589-5948, |
| | 5614-5861, 5620-5919, 5639-5888, 5654-5943, 5661-5941, 5673-5924, 5692-5936, 5692-5948, 5699-5952, 5701-5947, |
| | 5702-5948, 5715-5937, 5755-5931, 5777-5945 |
| 66/7504326CB1/ | 1-474, 1-3469, 44-606, 272-929, 284-351, 284-607, 284-711, 284-712, 284-713, 284-720, 284-759, 284-767, 284-768, |
| 3665 | 284-780, 284-796, 284-865, 286-875, 288-632, 289-941, 291-843, 292-746, 292-778, 293-765, 293-826, 293-831, |
| | 295-600, 295-651, 295-691, 295-782, 295-796, 295-851, 295-904, 295-914, 295-966, 296-831, 296-917, 302-646, |
| | 302-720, 302-731, 308-439, 314-582, 321-560, 324-982, 332-493, 351-676, 367-707, 367-838, 367-932, 367-1068, |
| | 375-503, 383-742, 384-636, 385-653, 386-916, 407-776, 421-1067, 433-917, 452-1137, 466-979, 516-1095, |
| | 516-1278, 517-926, 526-1148, 533-1217, 541-1148, 606-3197, 935-1672, 1056-1721, 1127-1668, 1181-1763, 1218-1816, |
| | 1265-1940, 1269-2145, 1271-1713, 1278-1903, 1312-2206, 1319-1897, 1319-1907, 1319-3201, 1327-1530, |
| | 1411-1970, 1497-1759, 1498-1792, 1505-1719, 1512-1777, 1517-1653, 1517-1734, 1517-1929, 1517-1980, 1517-2026, |
| | 1517-2033, 1517-2035, 1517-2055, 1517-2078, 1517-2083, 1517-2087, 1517-2123, 1521-2010, 1536-2197, |
| | 1543-1838, 1557-1835, 1566-1838, 1567-1832, 1570-1965, 1577-2141, 1587-2053, 1621-1886, 1634-2093, 1634-2109, |
| | 1635-2153, 1671-1872, 1685-2243, 1689-1931, 1689-1996, 1699-2304, 1704-2281, 1720-2337, |
| | 1731-2307, 132-2381, 1759-2016, 1762-2343, 1790-2081, 1793-2554, 1796-2075, 1805-1878, 1811-2225, 1844-2347, |
| | 1853-2347, 1875-2503, 1886-2558, 1891-2540, 1896-2180, 1899-2182, 1902-2526, 1903-2528, 1905-2185, |
| | 1907-2151, 1916-2632, 1925-2431, 1925-2460, 1926-2527, 1932-2558, 1944-2187, 1955-2199, 1957-2252, 1971-2207, |
| | 1973-2515, 1974-2622, 1979-2622, 1988-2551, 1998-2251, 2004-2634, 2009-2511, 2009-2516, 2011-2548, |
| | 2013-2473, 2013-2626, 2017-2565, 2020-2511, 2020-2516, 2030-2274, 2030-2628, 2054-2529, 2059-2648, 2062-2620, |
| | 2067-2623, 2079-2619, 2079-2628, 2079-2629, 2086-2648, 2110-2323, 2112-2669, 2126-2629, 2134-2623, |
| | 2139-2380, 2146-2648, 2146-2714, 2156-2629, 2158-2609, 2161-2635, 2163-2631, 2166-2629, 2168-2629, 2175-2632, |
| | 2178-2631, 2194-2646, 2195-2629, 2199-2633, 2204-2379, 2210-2629, 2220-2635, 2233-2629, 2234-2632, |
| | 2235-2631, 2366-2998, 2381-2965, 2407-3164, 2429-2955, 2498-3200, 2521-2971, 2565-2801, 2565-2809, 2599-3101, |
| | 2602-2874, 2624-3153, 2630-3098, 2630-3142, 2634-3211, 2641-3240, 2657-3195, 2658-3354, 2671-3224, |
| | 2681-3337, 2683-3284, 2685-3223, 2723-3164, 2776-3280, 2782-2899, 2809-3057, 2814-3260, 2823-3372, |
| | 2828-3197, 2836-3200, 2840-3304, 2843-3267, 2866-3469, 2870-3198, 2874-3197, 2880-3202, 2894-3195, 2899-3196, |
| | 2918-3197, 2919-3377, 2925-3185, 2935-3202, 2935-3252, 2938-3574, 2940-3343, 2949-3211, 2953-3440, |
| | 2965-3665, 2967-3205, 2976-3256, 2992-3240, 2992-3518, 3002-3259, 3008-3611, 3009-3256, 3013-3304, 3017-3367, |
| | 3018-3268, 3018-3397, 3031-3665, 3037-3605, 3039-3323, 3039-3585, 3040-3282, 3045-3289, 3050-3306, |
| | 3055-3252, 3060-3261, 3062-3344, 3066-3231, 3069-3231, 3071-3231, 3071-3309, 3074-3231, 3075-3318, 3075-3354, |
| | 3075-3496, 3079-3404, 3084-3237, 3086-3416 |
| 67/7504388CB1/ | 1-511, 18-424, 18-491, 42-766, 74-892, 223-901, 223-912, 261-1040, 282-850, 319-573, 334-818, 389-633, 395-665, |
| 1260 | 402-636, 417-630, 417-971, 449-715, 449-1126, 456-732, 468-702, 474-1215, 539-888, 540-1204, 542-1207, |
| | 544-888, 563-1235, 605-1200, 641-1234, 644-935, 684-1254, 687-1241, 690-1234, 691-1231, 692-1240, 712-1234, |
| | 715-1201, 715-1233, 730-1229, 733-1254, 748-1258, 757-1238, 758-1254, 767-1240, 769-1010, 772-1253, 773-1240, |
| | 773-1258, 774-1258, 786-1238, 796-1234, 797-1233, 803-1240, 804-1253, 817-1255, 824-1243, 833-1173, |
| | 833-1240, 835-1230, 840-1258, 846-1233, 848-1229, 853-1003, 853-1253, 882-1236, 887-1234, 901-1240, 904-1056, |
| | 905-1233, 905-1234, 921-1231, 923-1245, 923-1252, 923-1260, 926-1245, 927-1234, 929-1245, 931-1176, |
| | 931-1233, 936-1190, 939-1252, 944-1260, 947-1233, 951-1201, 968-1233, 975-1234, 989-1255, 1010-1234, 1016-1240, |
| | 1023-1258, 1024-1251, 1026-1240, 1029-1258, 1034-1257, 1046-1240, 1076-1240 |
| 68/2828380CB1/ | 1-422, 1-626, 1-804, 13-652, 47-529, 114-616, 318-939, 320-953, 327-948, 327-954, 354-953, 400-953, 664-1209, |
| 3907 | 664-1300, 814-923, 815-838, 815-839, 815-905, 815-912, 815-923, 862-1482, 863-916, 863-922, 863-923, 863-997, |
| | 863-1084, 863-1091, 863-1129, 863-1157, 863-1176, 863-1252, 864-916, 864-923, 864-1092, 864-1158, 876-934, |
| | 884-915, 884-916, 884-923, 884-1059, 884-1176, 885-1251, 885-1252, 888-934, 889-1480, 890-1091, 890-1175, |
| | 890-1176, 913-1091, 971-1430, 983-1176, 985-1252, 985-1671, 988-1135, 988-1157, 988-1252, 988-1319, 988-1333, |
| | 988-1539, 988-1622, 992-1157, 992-1246, 992-1321, 994-1176, 1016-1176, 1021-1402, 1029-1157, 1029-1175, |
| | 1029-1176, 1043-1085, 1043-1153, 1043-1176, 1043-1252, 1043-1259, 1043-1281, 1043-1420, 1043-1421, |
| | 1043-1589, 1048-1252, 1049-1085, 1049-1132, 1049-1154, 1049-1241, 1050-1126, 1050-1155, 1050-1174, 1050-1175, |
| | 1054-1157, 1054-1456, 1054-1505, 1054-1514, 1056-1420, 1057-1420, 1066-1174, 1066-1175, 1067-1260, |
| | 1067-1562, 1073-1175, 1074-1321, 1075-1321, 1107-1153, 1107-1420, 1107-1672, 1115-1175, 1116-1414, 1122-1176, |
| | 1122-1260, 1126-1420, 1138-1176, 1140-1561, 1141-1175, 1141-1176, 1142-1176, |
| | 1199-1259, 1199-1285, 1199-1493, 1199-1588, 1199-1818, 1200-1252, 1200-1260, 1202-1260, 1208-1321, 1208-1588, |
| | 1210-1252, 1210-1260, 1211-1260, 1216-1420, 1217-1420, 1224-1420, 1232-1321, 1232-1816, 1233-1260, |
| | 1235-1262, 1249-1420, 1283-1420, 1284-1345, 1284-1841, 1292-1429, 1292-1470, 1292-1513, 1292-1577, 1292-1588, |
| | 1302-1336, 1302-1337, 1302-1395, 1302-1552, 1302-1554, 1302-1577, 1302-1585, 1302-1588, 1302-1674, |
| | 1302-1681, 1302-1732, 1302-1734, 1302-1959, 1306-1465, 1306-1598, 1306-1757, 1306-1768, 1306-1850, 1321-1643, |
| | 1321-2044, 1330-1420, 1335-1420, 1357-1756, 1371-1597, 1371-1745, 1372-1792, 1373-1504, 1373-1585, |
| | 1373-1673, 1373-1674, 1373-1678, 1373-1925, 1380-1588, 1385-1420, 1385-1636, 1385-1756, 1386-1498, 1386-1504, |
| | 1386-1674, 1388-1420, 1390-1850, 1408-1577, 1408-1727, 1408-1897, 1408-2091, 1409-1504, 1410-1669, |
| | 1410-1756, 1411-1669, 1417-1672, 1424-1828, 1428-2009, 1442-1840, 1451-1840, 1452-1584, 1455-1840, 1459-1493, |
| | 1459-1750, 1459-1756, 1462-1504, 1462-1756, 1463-1822, 1463-1902, 1468-1722, 1472-1504, 1472-1512, |
| | 1474-1573, 1474-1588, 1474-1633, 1474-1646, 1474-1936, 1477-1504, |
| | 1492-1588, 1492-1661, 1492-1756, 1492-1811, 1492-1984, 1492-1986, 1492-2165, 1493-1588, 1494-1837, 1494-1840, |
| | 1495-1825, 1498-1588, 1501-1765, 1508-1912, 1512-2107, 1526-1588, 1535-1588, 1535-1924, 1536-1585, |
| | 1536-1588, 1536-1674, 1536-1825, 1536-1840, 1538-1840, 1539-1588, 1542-1588, 1546-1585, 1546-1840, 1547-1756, |
| | 1552-1806, 1579-1674, 1579-1828, 1579-1924, 1585-1849, 1595-1997, 1596-2161, 1607-2008, 1619-2008, |
| | 1624-1895, 1624-2020, 1625-1661, 1625-1668, 1625-1669, 1625-1897, 1625-1924, 1626-1673, 1626-1674, 1628-1756, |
| | 1630-1669, 1641-1890, 1641-1909, 1641-2008, 1642-1738, 1642-1840, 1642-2065, 1642-2068, 1642-2092, |
| | 1648-1674, 1653-2259, 1660-1971, 1661-1756, 1662-1999, 1662-2008, 1663-1999, 1666-1756, 1669-1933, 1685-2077, |
| | 1688-2260, 1695-1745, 1703-1747, 1703-1753, 1703-1756, 1703-1764, 1703-1828, 1703-1924, 1703-1997, |
| | 1703-2129, 1703-2161, 1704-1825, 1704-1840, 1704-2002, 1704-2008, 1706-2008, 1710-1753, 1710-2095, 1710-2184, |
| | 1711-2260, 1714-2008, 1715-1825, 1715-2074, 1715-2092, 1716-2008, 1720-1756, 1720-1971, 1721-1756, |
| | 1726-2173, 1729-1756, 1730-1756, 1744-2063, 1744-2427, 1745-1840, 1746-2099, 1746-2161, 1747-2083, |
| | 1753-2017, 1760-2167, 1764-2345, 1778-2008, 1787-2008, 1788-1840, 1788-1848, 1788-1912, 1788-1924, 1788-2099, |
| | 1788-2184, 1793-2184, 1794-2008, 1796-1897, 1798-2184, 1799-2008, 1804-2058, 1805-1840, 1810-2260, |
| | 1813-1840, 1830-1924, 1830-2174, 1830-2184, 1831-2170, 1832-2092, 1832-2161, 1832-2428, 1834-1924, 1837-2092, |
| | 1839-1924, 1844-2249, 1854-2435, 1870-2161, 1870-2266, 1870-2341, 1872-1924, 1872-1932, 1872-2008, |
| | 1872-2083, 1872-2215, 1872-2297, 1877-2008, 1878-2267, 1882-2008, 1883-2161, 1888-2092, 1894-2353, 1899-1924, |
| | 1914-2008, 1914-2395, 1914-2400, 1915-2254, 1921-2165, 1928-2329, 1932-2428, 1955-2184, 1956-1999, |
| | 1956-2008, 1956-2099, 1956-2161, 1956-2183, 1956-2184, 1956-2341, 1961-2173, 1961-2341, 1962-2184, 1963-2428, |
| | 1966-2341, 1973-2008, 1973-2105, 1973-2165, 1973-2184, 1976-2008, 1978-2428, 1981-2008, 1996-2428, |
| | 1997-2184, 2004-2184, 2005-2260, 2015-2260, 2023-2428, 2024-2184, 2025-2260, 2025-2428, 2027-2289, 2027-2628, |
| | 2030-2428, 2038-2626, 2038-2781, 2043-2718, 2046-2184, 2050-2089, 2050-2092, 2050-2184, 2051-2410, |
| | 2052-2103, 2052-2110, 2056-2260, 2058-2092, 2058-2752, 2062-2430, 2064-2839, 2065-2110, |
| | 2075-2176, 2075-2183, 2095-2500, 2121-2268, 2124-2183, 2124-2329, 2124-2347, 2124-2428, 2124-2435, 2129-2370, |
| | 2130-2268, 2134-2734, 2134-2821, 2142-2184, 2142-2394, 2142-2430, 2149-2183, 2207-2267, 2207-2520, |
| | 2208-2260, 2208-2268, 2217-2509, 2218-2260, 2218-2261, 2218-2268, 2219-2268, 2219-2435, 2220-2520, 2224-2260, |
| | 2224-2994, 2225-2503, 2229-2967, 2232-2519, 2234-2268, 2234-2520, 2241-2268, 2257-2504, 2289-2951, |
| | 2292-2344, 2292-2428, 2292-2430, 2292-2520, 2300-2428, 2302-2344, 2302-2435, 2310-2353, 2310-2402, 2310-2428, |
| | 2310-2435, 2314-2428, 2316-2344, 2329-2428, 2334-2428, 2335-2428, 2338-2574, 2338-2744, 2343-2939, |
| | 2363-2512, 2363-2520, 2368-2520, 2375-2435, 2379-2428, 2391-2520, 2405-2520, 2406-2435, 2411-2435, 2455-3003, |
| | 2455-3125, 2466-2509, 2466-2513, 2466-2520, 2470-2520, 2484-2513, 2485-2520, 2486-2510, 2486-2520, |
| | 2508-2775, 2508-2892, 2509-3011, 2521-3013, 2523-2966, 2550-2646, 2565-2818, 2568-3021, 2605-2874, 2645-3185, |
| | 2645-3295, 2779-3493, 2966-3641, 3056-3637, 3071-3522, 3079-3522, 3147-3168, 3162-3907, 3171-3511, |
| | 3203-3423, 3261-3288, 3283-3524 |
| 69/6456919CB1/ | 1-279, 1-473, 29-631, 257-837, 265-837, 325-735, 413-925, 415-889, 597-683, 633-688, 641-679, 641-688, 642-678, |
| 3313 | 642-679, 642-688, 643-695, 644-695, 722-856, 722-925, 722-937, 739-835, 739-856, 739-916, 739-937, 739-1026, |
| | 739-1108, 759-940, 770-1476, 798-838, 798-839, 798-851, 798-856, 798-904, 798-907, 798-916, 798-936, |
| | 798-937, 798-1020, 798-1026, 798-1027, 798-1105, 798-1309, 798-1343, 798-1365, 798-1368, 800-937, 809-838, |
| | 809-863, 809-937, 810-838, 810-1026, 810-1105, 810-1356, 812-863, 835-1026, 840-1026, 844-937, 856-1285, 870-1430, |
| | 874-1024, 875-940, 875-1440, 878-941, 878-1108, 878-1448, 880-1327, 882-937, 882-1026, 882-1449, 886-937, |
| | 886-1024, 887-937, 891-937, 892-937, 893-1024, 919-1609, 927-988, 927-1019, 928-1019, 947-1546, 954-1368, |
| | 958-1108, 962-1368, 963-1105, 965-1026, 966-1025, 966-1026, 966-1104, 966-1105, 968-1105, 969-1609, |
| | 972-1026, 973-1532, 977-1026, 996-1026, 1000-1368, 1005-1105, 1043-1108, 1047-1480, 1048-1480, 1050-1105, |
| | 1064-1574, 1074-1667, 1079-1105, 1079-1109, 1082-1661, 1093-1371, 1115-1623, 1125-1259, 1125-1361, |
| | 1128-1161, 1128-1178, 1128-1220, 1128-1255, 1128-1261, 1128-1311, 1128-1334, 1128-1364, 1128-1389, 1128-1411, |
| | 1128-1414, 1128-1415, 1128-1432, 1128-1449, 1129-1449, 1130-1449, 1131-1368, 1131-1449, 1144-1180, |
| | 1144-1183, 1144-1342, 1144-1343, 1144-1345, 1144-1445, 1144-1767, 1146-1175, 1146-1224, 1146-1363, 1146-1446, |
| | 1146-1692, 1150-1361, 1159-1336, 1160-1308, 1160-1390, 1160-1445, 1160-1448, 1164-1362, 1164-1380, |
| | 1164-1411, 1164-1448, 1164-1449, 1187-1722, 1205-1446, 1205-1448, 1205-1449, 1205-1480, 1206-1445, 1210-1277, |
| | 1210-1480, 1217-1258, 1217-1362, 1217-1446, 1218-1276, 1218-1277, 1218-1348, 1218-1361, 1218-1365, |
| | 1218-1368, 1218-1387, 1218-1425, 1218-1430, 1218-1446, 1218-1449, 1218-1489, 1218-1494, 1218-1495, 1218-1534, |
| | 1218-1539, 1218-1690, 1226-1341, 1226-1360, 1226-1429, 1229-1259, 1229-1277, 1229-1368, 1229-1448, |
| | 1230-1259, 1230-1266, 1230-1267, 1230-1277, 1230-1362, 1230-1370, 1230-1447, 1235-1277, 1239-1277, 1239-1449, |
| | 1241-1446, 1245-1449, 1247-1273, 1252-1480, 1255-1865, 1255-1942, 1255-1956, 1257-1480, 1257-1856, |
| | 1269-1480, 1270-1480, 1271-1744, 1271-1745, 1278-1870, 1283-1446, 1283-1449, 1283-1480, 1283-1889, |
| | 1286-1480, 1290-1448, 1292-1365, 1294-1361, 1296-1918, 1298-1449, 1298-1480, 1298-1945, 1302-1336, 1302-1346, |
| | 1302-1368, 1302-1427, 1302-1441, 1302-1444, 1302-1446, 1302-1448, 1302-1449, 1302-1483, 1304-1441, |
| | 1305-1360, 1308-1343, 1308-1350, 1308-1365, 1308-1382, 1308-1396, 1308-1434, 1310-1357, 1311-1346, 1312-1365, |
| | 1312-1449, 1315-1426, 1315-1428, 1316-1444, 1316-1483, 1319-1360, 1322-1396, 1323-1365, 1323-1480, |
| | 1323-1913, 1329-1480, 1332-1365, 1338-1767, 1355-1448, 1367-1979, 1376-1449, 1384-1448, 1392-1445, 1392-1446, |
| | 1392-1448, 1392-1449, 1392-1480, 1393-1446, 1394-1446, 1395-1421, 1395-1423, 1395-1427, 1395-1441, |
| | 1396-1480, 1396-1770, 1397-1425, 1397-1444, 1397-1445, 1397-1451, 1398-1444, 1398-1476, 1399-1444, 1400-1444, |
| | 1403-1444, 1404-1446, 1404-1483, 1407-1449, 1407-1792, 1416-1446, 1416-1874, 1422-1449, 1426-1801, |
| | 1450-2133, 1460-1948, 1479-1690, 1482-1615, 1482-1724, 1482-1780, 1482-1805, 1483-1971, 1493-1594, 1493-1606, |
| | 1493-1683, 1493-1712, 1493-1801, 1493-1808, 1500-1805, 1504-1606, 1504-1613, 1504-1615, 1504-1717, |
| | 1504-1872, 1513-1609, 1513-1694, 1518-1718, 1518-1720, 1518-1796, 1518-1808, 1518-1908, 1521-1892, |
| | 1522-1718, 1522-1908, 1532-1892, 1555-1923, 1560-1805, 1563-1665, 1563-1802, 1563-1879, 1563-1920, 1564-1613, |
| | 1564-1615, 1564-1920, 1571-1609, 1572-1608, 1572-1614, 1572-1615, 1572-1616, 1572-1713, 1572-1720, |
| | 1572-1808, 1572-1819, 1572-1879, 1572-1908, 1574-1616, 1581-1607, 1581-1613, 1581-1615, 1581-1616, 1582-1613, |
| | 1584-1613, 1586-1802, 1593-1908, 1619-2134, 1629-1808, 1639-1805, 1639-1819, 1639-1908, 1639-1913, |
| | 1639-1920, 1642-1805, 1642-1920, 1643-1808, 1649-2231, 1653-1805, 1654-1805, 1654-1819, 1654-1920, 1655-1720, |
| | 1656-1808, 1658-1695, 1658-1702, 1658-1713, 1658-1716, 1658-1717, 1658-1718, 1658-1724, 1658-1745, |
| | 1658-1785, 1658-1803, 1658-1810, 1658-1870, 1658-1872, 1658-1873, 1658-1879, 1658-1885, 1658-1916, 1658-1923, |
| | 1658-1934, 1658-1969, 1658-2011, 1658-2043, 1658-2053, 1658-2125, 1658-2155, 1658-2160, 1660-1808, |
| | 1664-1695, 1664-1704, 1664-1710, 1664-1717, 1664-1720, 1664-1724, 1664-1767, 1664-1775, 1664-1788, 1664-1805, |
| | 1664-1811, 1664-1839, 1668-1720, 1671-1780, 1672-1718, 1672-1873, 1679-1720, 1679-1839, 1698-1805, |
| | 1732-1805, |
| | 1748-1804, 1748-1824, 1748-1836, 1748-1851, 1748-1854, 1748-1879, 1748-1895, 1748-1896, 1748-1908, 1748-1920, |
| | 1748-1923, 1748-2160, 1748-2215, 1748-2236, 1748-2358, 1752-1805, 1758-1800, 1758-1867, 1758-1868, |
| | 1758-1923, 1762-1872, 1763-1805, 1763-1885, 1763-2348, 1778-2160, 1795-1908, 1807-2358, 1822-1923, 1832-1866, |
| | 1832-1873, 1832-1885, 1832-1892, 1832-1894, 1832-1920, 1834-1871, 1836-1884, 1837-1884, 1838-1884, |
| | 1838-1885, 1838-2205, 1839-1884, 1843-1884, 1847-1923, 1847-2223, 1856-2236, 1862-1920, 1866-2442, 1899-2358, |
| | 1900-2358, 1919-2136, 1923-2358, 1935-2067, 1935-2159, 1935-2218, 1935-2224, 1935-2236, 1937-2236, |
| | 1941-2049, 1941-2050, 1941-2052, 1941-2152, 1941-2405, 1946-2099, 1946-2118, 1946-2147, 1946-2157, 1946-2218, |
| | 1950-2048, 1950-2068, 1950-2127, 1955-2157, 1955-2218, 1981-2236, 1983-2218, 1987-2218, 1988-2153, |
| | 1988-2370, 1988-2385, 1988-2489, 1990-2524, 2000-2216, 2000-2236, 2000-2315, 2000-2385, 2001-2064, 2001-2170, |
| | 2001-2385, 2005-2106, 2005-2489, |
| | 2009-2063, 2009-2067, 2009-2134, 2009-2138, 2009-2152, 2009-2159, 2009-2163, 2009-2170, 2009-2171, 2009-2187, |
| | 2009-2189, 2009-2190, 2009-2202, 2009-2218, 2009-2224, 2009-2655, 2022-2050, 2022-2057, 2022-2061, |
| | 2022-2062, 2022-2067, 2022-2074, 2022-2153, 2022-2163, 2022-2170, 2022-2202, 2022-2224, 2022-2236, 2022-2320, |
| | 2022-2332, 2022-2346, 2022-2356, 2022-2489, 2022-2559, 2023-2067, 2023-2216, 2025-2055, 2025-2067, |
| | 2025-2127, 2025-2134, 2025-2137, 2025-2138, 2025-2156, 2025-2177, 2025-2190, 2025-2236, 2025-2320, 2025-2384, |
| | 2025-2385, 2025-2391, 2026-2074, 2027-2064, 2027-2133, 2027-2157, 2027-2218, 2027-2449, 2027-2483, |
| | 2027-2489, 2030-2067, 2030-2190, 2030-2346, 2033-2218, 2036-2236, 2036-2346, 2038-2064, 2039-2067, 2045-2190, |
| | 2046-2236, 2048-2236, 2048-2385, 2061-2346, 2064-2346, 2066-2346, 2070-2346, 2080-2236, 2080-2329, |
| | 2080-2358, 2080-2385, 2084-2649, 2085-2236, 2087-2236, 2089-2236, 2089-2385, 2091-2218, 2093-2127, 2093-2132, |
| | 2093-2137, 2093-2148, 2093-2151, 2093-2170, 2093-2213, 2093-2218, 2093-2235, 2093-2236, 2093-2489, |
| | 2093-2527, 2093-2602, 2093-2655, 2093-2742, 2095-2218, 2099-2190, 2103-2190, 2103-2346, |
| | 2106-2217, 2106-2218, 2113-2190, 2114-2190, 2114-2224, 2114-2385, 2120-2329, 2120-2385, 2123-2190, 2129-2236, |
| | 2132-2489, 2158-2733, 2168-2236, 2169-2385, 2170-2720, 2171-2236, 2172-2236, 2172-2389, 2172-2391, |
| | 2176-2236, 2176-2329, 2177-2489, 2177-2620, 2177-2741, 2181-2346, 2182-2218, 2182-2346, 2183-2236, 2185-2489, |
| | 2185-2655, 2188-2488, 2188-2489, 2189-2214, 2189-2216, 2189-2217, 2189-2218, 2189-2236, 2189-2329, |
| | 2189-2389, 2189-2391, 2189-2406, 2189-2470, 2189-2482, 2189-2489, 2189-2825, 2190-2301, 2190-2302, 2190-2304, |
| | 2190-2489, 2190-2686, 2191-2384, 2191-2551, 2195-2236, 2198-2489, 2204-2489, 2210-2489, 2239-2871, |
| | 2247-2489, 2248-2489, 2255-2385, 2257-2804, 2258-2483, 2258-2634, 2258-2655, 2258-2689, 2259-2704, 2260-2321, |
| | 2260-2655, 2261-2314, 2261-2321, 2261-2348, 2261-2370, 2261-2373, 2261-2381, 2261-2383, 2261-2385, |
| | 2261-2406, 2261-2478, 2261-2482, 2261-2486, 2261-2817, 2261-2829, 2263-2385, 2279-2321, 2279-2385, 2279-2489, |
| | 2279-2580, 2279-2634, 2279-2739, 2281-2391, 2281-2477, 2282-2391, 2282-2489, 2288-2689, 2293-2519, |
| | 2293-2689, 2294-2655, 2295-2489, 2298-2358, 2300-2655, 2312-2655, 2313-2654, 2313-2655, 2319-2985, |
| | 2324-2470, 2324-2472, 2324-2660, 2324-2740, 2334-2382, 2334-2552, 2337-2489, 2337-2569, 2337-2655, 2340-2829, |
| | 2341-2385, 2341-2463, 2341-2823, 2341-2825, 2343-2785, 2344-2382, 2344-2489, 2344-2741, 2344-2825, |
| | 2345-2384, 2345-2386, 2345-2489, 2345-2655, 2350-2484, 2350-2489, 2350-2823, 2366-2655, 2375-2653, 2375-2825, |
| | 2415-2823, 2416-2569, 2416-2689, 2417-2829, 2420-2466, 2420-2468, 2420-2473, 2420-2520, 2420-2527, |
| | 2420-2569, 2420-2633, 2420-2655, 2420-2720, 2420-2742, 2420-2747, 2420-2773, 2420-2803, 2421-3067, 2428-2823, |
| | 2429-2489, 2429-2569, 2429-2655, 2429-2739, 2431-2489, 2437-2489, 2439-2655, 2443-2685, 2452-2601, |
| | 2452-2655, 2452-2738, 2452-2741, 2458-2655, 2458-2741, 2459-2566, 2459-2569, 2459-2627, 2459-2655, 2459-2725, |
| | 2459-2742, 2459-2743, 2459-2802, 2459-2815, 2459-2816, 2459-2829, 2459-2845, 2459-2848, 2462-3082, |
| | 2468-2527, 2468-2689, 2468-2880, 2475-2689, 2478-2932, 2480-2932, 2481-2741, 2481-2822, 2481-3067, 2488-2980, |
| | 2491-2741, 2493-2767, 2493-3043, 2494-2689, 2494-2773, 2497-2991, 2500-2742, 2504-2823, 2505-2655, |
| | 2509-2655, 2509-2689, 2513-2578, 2513-2629, 2513-2655, 2513-2804, 2513-2818, 2513-2825, 2513-2845, |
| | 2518-2688, 2518-2711, 2518-2741, 2518-2776, 2518-2785, 2518-2791, 2518-2806, 2518-2829, 2518-2847, 2523-2569, |
| | 2524-2634, 2524-2848, 2525-2848, 2526-3086, 2532-2654, 2532-2739, 2532-2741, 2532-2804, 2534-2739, |
| | 2534-2825, 2537-2748, 2537-2825, 2538-2763, 2538-2934, 2545-2825, 2548-2742, 2550-2742, 2550-2825, 2556-2825, |
| | 2562-2825, 2563-2825, 2563-3048, 2566-2993, 2568-3039, 2572-2969, 2573-2825, 2575-2825, 2576-2742, |
| | 2576-2804, 2581-2823, 2582-2825, 2584-2804, 2586-2803, 2587-2741, 2587-2742, 2591-2627, 2591-2742, 2591-2764, |
| | 2591-2815, 2595-2717, 2595-2734, 2595-2823, 2595-2829, 2597-2734, 2598-2734, 2601-2654, 2601-2689, |
| | 2601-2691, 2601-2727, 2605-2689, 2605-3048, 2613-2713, 2613-2720, 2613-2722, 2613-2804, 2616-2689, 2616-2773, |
| | 2616-2825, 2618-2823, 2622-2825, 2641-2823, 2641-2825, 2646-2823, 2647-2823, 2647-2825, 2650-2825, |
| | 2650-2829, 2655-2825, 2656-2825, 2657-2823, 2658-3313, 2659-2823, 2660-2825, 2664-2823, 2664-2825, 2665-2829, |
| | 2669-2741, 2671-2848, 2672-2823, 2675-2741, 2675-2825, 2675-2829, |
| | 2679-2714, 2679-2715, 2679-2723, 2679-2734, 2679-2737, 2679-2739, 2679-2741, 2679-2799, 2679-2803, 2679-2806, |
| | 2679-2818, 2679-2829, 2679-2840, 2681-2862, 2682-2818, 2685-2742, 2686-2818, 2687-2823, 2689-2742, |
| | 2689-2825, 2692-2803, 2692-2804, 2693-2829, 2700-2742, 2700-2845, 2709-2742, 2717-2825, 2732-3009, 2745-2825, |
| | 2753-2825, 2755-2800, 2755-2805, 2755-2806, 2755-2818, 2755-2822, 2755-2923, 2763-2802, 2763-2816, |
| | 2763-2818, 2763-2825, 2763-2829, 2767-2823, 2768-2818, 2768-2825, 2768-2829, 2772-2806, 2773-2825, 2775-2804, |
| | 2780-2817, 2784-2818, 2792-2818 |
| 70/7502244CB1/ | 1-695, 549-838, 603-851, 667-805, 692-1116, 697-805, 889-1520, 934-1614, 940-1614, 1375-2044, 1492-2024, |
| 2095 | 1581-1867, 1581-2083, 1581-2095, 1584-2066, 1616-1913, 1616-2068, 1616-2095, 1633-2095, 1640-2082, 1647-2080, |
| | 1648-2081, 1676-2081, 1680-2081, 1737-2082, 1776-2081, 1789-2075, 1790-2081, 1826-2082, 1832-2081, |
| | 1834-2083, 1848-2081, 1856-2050, 1881-2078, 1943-2095 |
| 71/7498718CB1/ | 1-569, 1-679, 1-704, 310-996, 331-546, 368-996, 392-2109, 393-794, 393-798, 393-838, 393-902, 393-916, 393-983, |
| 2109 | 393-1034, 393-1042, 393-1094, 414-996, 698-1375, 752-1375, 763-1375, 787-1375, 833-1375, 836-1375, 892-1375, |
| | 901-1375, 985-1375, 1049-1375, 1144-1209, 1151-1200, 1151-1205, 1161-1209, 1164-1200, 1256-1603, 1256-1666, |
| | 1256-1681, 1256-1769, 1284-1435, 1284-1795, 1284-1936, 1297-1375, 1297-1501, 1297-1520, 1297-1879, |
| | 1309-1619, 1309-2109, 1310-1357, 1310-1459, 1310-1585, 1310-1709, 1310-1853, 1310-1961, 1320-1417, 1381-1459, |
| | 1381-2109, 1394-1694, 1394-2049, 1416-1452, 1453-1543, 1453-2108, 1488-1777, 1488-2108, 1562-1609, |
| | 1562-1855, 1562-2109, 1592-1709, 1592-2109, 1654-1946, 1654-2109, 1665-1713, 1676-1795, 1676-2090, 1739-1793, |
| | 1740-2034, 1740-2090, 1814-1879, 1814-2093, 1816-1881, 1885-1963, 1885-2093, 1898-2093, 2003-2049, |
| | 2003-2093, 2008-2044 |
| 72/6259308CB1/ | 1-484, 305-458, 352-1017, 361-453, 361-482, 390-992, 486-781, 486-809, 486-848, 486-851, 486-861, 486-927, 486-933, |
| 1440 | 486-950, 486-957, 486-960, 486-961, 486-967, 486-970, 486-971, 486-974, 486-975, 486-981, 486-989, 493-872, |
| | 494-896, 495-561, 495-581, 495-749, 496-749, 496-976, 504-880, 504-919, 504-941, 504-956, 504-963, 505-939, |
| | 508-957, 517-892, 519-917, 521-762, 521-861, 521-882, 521-978, 521-979, 521-981, 524-932, 524-975, 526-865, |
| | 528-978, 529-984, 530-932, 532-907, 536-1004, 538-1001, 539-997, 547-1005, 560-957, 564-941, 571-860, |
| | 575-984, 580-989, 583-914, 585-922, 590-1128, 590-1135, 590-1194, 591-1119, 592-916, 592-982, 594-912, 594-1237, |
| | 595-908, 619-749, 619-1157, 626-1143, 626-1274, 630-1160, 630-1205, 640-811, 640-897, 640-941, 643-872, |
| | 643-916, 643-1110, 643-1151, 644-1173, 650-920, 650-1211, 658-877, 658-1087, 667-942, 668-1166, 675-931, 675-994, |
| | 675-1139, 675-1163, 675-1260, 678-1260, 683-899, 696-1187, 701-1108, 705-866, 711-1173, 717-984, 721-971, |
| | 721-1173, 725-997, 725-1173, 730-909, 736-1165, 744-871, 748-1318, 748-1439, 754-857, |
| | 781-968, 781-1074, 781-1297, 792-1105, 800-1354, 817-1312, 819-1028, 819-1096, 819-1378, 820-903, 830-1279, |
| | 831-1271, 835-1078, 836-1177, 838-1190, 842-1299, 847-1023, 850-1153, 851-1111, 858-1430, 860-1079, 868-1084, |
| | 874-1116, 875-1289, 880-1346, 889-1090, 889-1216, 890-1084, 891-1439, 896-1102, 896-1297, 899-1133, |
| | 909-1277, 925-1439, 927-1157, 927-1431, 931-1149, 938-994, 938-1218, 976-1268, 980-1357, 982-1235, 994-1308, |
| | 998-1241, 1003-1440, 1013-1300, 1021-1128, 1021-1280, 1021-1308, 1021-1440, 1022-1440, 1023-1440, 1024-1440, |
| | 1034-1288, 1042-1438, 1051-1440, 1070-1431, 1079-1439, 1080-1324, 1081-1373, 1084-1266, 1113-1320, |
| | 1128-1331, 1131-1320, 1139-1304, 1149-1396, 1161-1408, 1164-1440, 1194-1297, 1196-1371, 1206-1440, 1207-1294, |
| | 1219-1440, 1234-1440, 1254-1427, 1263-1440, 1264-1440, 1278-1436, 1294-1439, 1295-1440, 1322-1440, |
| | 1325-1440, 1326-1440, 1331-1439, 1332-1439, 1332-1440, 1335-1439 |
| 73/7504104CB1/ | 1-652, 436-715, 436-866, 436-888, 436-889, 436-953, 436-1033, 436-1321, 439-2787, 469-1194, 491-1102, 538-1315, |
| 2794 | 580-758, 686-1219, 736-858, 764-1308, 789-1278, 804-1509, 817-1606, 817-1617, 819-1377, 843-1471, 854-1322, |
| | 857-1478, 891-1455, 902-1304, 936-1527, 956-1437, 959-1316, 961-1520, 962-1578, 967-1593, 968-1343, |
| | 972-1479, 975-1531, 979-1267, 986-1483, 997-1228, 997-1583, 999-1527, 1014-1479, 1021-1519, 1023-1635, 1028-1526, |
| | 1051-1585, 1055-1594, 1058-1648, 1063-1334, 1065-1335, 1069-1756, 1070-1890, 1072-1443, 1076-1651, |
| | 1080-1668, 1089-1580, 1095-1220, 1115-1732, 1124-1685, 1125-1559, 1125-1607, 1129-1890, 1134-1890, 1137-1748, |
| | 1141-1568, 1163-1890, 1165-1890, 1182-1760, 1195-1454, 1197-1846, 1204-1801, 1223-1491, 1233-1887, |
| | 1245-1505, 1247-1890, 1271-1756, 1278-1565, 1278-1890, 1290-1562, 1294-1517, 1295-1790, 1296-1910, 1300-1940, |
| | 1327-1672, 1330-1899, 1333-1790, 1339-1623, 1353-1601, 1360-2001, 1369-2017, 1395-2017, 1399-1698, |
| | 1399-2035, 1413-1863, 1413-1955, 1416-1968, 1426-2068, 1430-2016, 1438-1732, 1438-2039, |
| | 1440-1636, 1442-1936, 1450-1733, 1450-2044, 1459-2055, 1470-1817, 1473-1744, 1489-2046, 1515-2086, 1547-1966, |
| | 1548-2061, 1563-2065, 1564-2225, 1573-1777, 1576-2111, 1584-2151, 1597-1941, 1599-1795, 1599-1852, |
| | 1604-1986, 1621-2514, 1642-2027, 1642-2028, 1642-2095, 1643-1920, 1667-2036, 1669-2233, 1677-1974, 1688-2063, |
| | 1690-1986, 1701-2408, 1710-2264, 1712-1793, 1731-2449, 1745-2034, 1747-2530, 1748-2560, 1760-2363, |
| | 1775-1896, 1832-2131, 1833-2113, 1836-2313, 1840-2152, 1855-2024, 1863-2557, 1870-2125, 1870-2551, 1873-2486, |
| | 1883-2396, 1891-2724, 1897-2040, 1897-2460, 1903-2421, 1908-2163, 1940-2555, 1944-2606, 1947-2154, |
| | 1950-2603, 1953-2445, 1959-2193, 1960-2613, 1962-2160, 1971-2609, 1973-2165, 1979-2579, 1989-2602, 1993-2458, |
| | 1995-2576, 1997-2624, 2003-2270, 2013-2496, 2039-2300, 2039-2611, 2047-2340, 2047-2582, 2047-2593, |
| | 2053-2323, 2059-2315, 2071-2626, 2077-2431, 2082-2507, 2085-2304, 2085-2606, 2086-2158, 2091-2629, 2098-2312, |
| | 2101-2438, 2118-2651, 2132-2624, 2133-2624, 2134-2631, 2138-2624, 2141-2613, 2147-2612, |
| | 2148-2624, 2151-2606, 2157-2597, 2168-2368, 2174-2606, 2174-2612, 2176-2600, 2177-2340, 2184-2597, 2184-2631, |
| | 2190-2624, 2200-2560, 2204-2460, 2206-2612, 2215-2630, 2218-2606, 2220-2612, 2221-2631, 2225-2595, |
| | 2227-2612, 2231-2597, 2237-2612, 2260-2606, 2262-2597, 2271-2736, 2276-2504, 2279-2548, 2285-2604, 2285-2613, |
| | 2286-2593, 2291-2611, 2308-2598, 2309-2567, 2329-2624, 2332-2597, 2338-2713, 2352-2629, 2358-2604, |
| | 2371-2633, 2400-2794, 2405-2794, 2411-2624, 2419-2676, 2431-2611, 2522-2613, 2522-2736, 2522-2763, 2540-2770, |
| | 2547-2782, 2630-2772 |
| 74/7504121CB1/ | 1-424, 1-468, 1-502, 1-569, 5-443, 14-490, 40-763, 110-564, 117-400, 157-457, 196-836, 202-609, 202-741, 202-755, |
| 2505 | 202-777, 202-817, 202-832, 202-834, 202-836, 203-777, 203-831, 203-832, 203-836, 222-2497, 230-569, 232-836, |
| | 327-598, 408-639, 408-680, 409-689, 411-698, 452-574, 452-686, 452-830, 452-955, 452-971, 452-1101, 485-1027, |
| | 560-1096, 606-836, 636-836, 690-1379, 706-1379, 715-1107, 743-1395, 771-1379, 818-1379, 831-1379, 857-1157, |
| | 857-1159, 857-1333, 857-1350, 857-1368, 857-1370, 857-1411, 857-1417, 857-1447, 857-1461, 857-1467, |
| | 877-1420, 881-1143, 896-1379, 898-1559, 922-1205, 922-1210, 923-1165, 954-1095, 963-1231, 979-1121, 980-1121, |
| | 981-1262, 992-1248, 992-1280, 1067-1375, 1071-1579, 1096-1184, 1096-1404, 1096-1488, 1096-1494, 1096-1667, |
| | 1125-1280, 1128-1565, 1150-1445, 1150-1556, 1150-1615, 1150-1829, 1150-1857, 1155-1731, 1160-1739, |
| | 1177-1494, 1185-1667, 1189-1667, 1208-1667, 1208-1860, 1252-1512, 1276-1964, 1277-1483, 1280-1557, 1282-1550, |
| | 1284-1550, 1284-1578, 1289-1581, 1292-1964, 1306-1553, 1322-1535, 1353-1821, 1358-1583, |
| | 1393-1991, 1396-1939, 1406-1652, 1406-2030, 1414-1660, 1435-2079, 1442-1679, 1458-1770, 1473-1785, 1475-2143, |
| | 1497-1667, 1516-2031, 1521-1815, 1522-1868, 1522-2048, 1538-2123, 1538-2132, 1542-2217, 1566-1799, |
| | 1566-2042, 1566-2182, 1566-2236, 1566-2251, 1570-1796, 1582-1801, 1585-2021, 1585-2079, 1585-2130, 1585-2148, |
| | 1586-2018, 1587-2244, 1593-2142, 1595-1892, 1595-1898, 1604-2209, 1610-2430, 1668-1964, 1668-1979, |
| | 1668-2079, 1668-2100, 1668-2105, 1668-2110, 1670-2062, 1680-2376, 1693-2241, 1694-2013, 1697-2273, 1708-1922, |
| | 1711-2375, 1714-1973, 1714-1982, 1730-2118, 1741-1998, 1741-2212, 1747-1983, 1765-2472, 1766-2014, |
| | 1772-2375, 1782-2375, 1786-2375, 1810-2302, 1816-2290, 1816-2450, 1818-2084, 1826-2078, 1852-2448, 1853-2475, |
| | 1857-2468, 1866-2350, 1866-2351, 1880-2137, 1880-2429, 1880-2463, 1881-2368, 1883-2468, 1892-2475, |
| | 1892-2501, 1893-2302, 1894-2499, 1901-2376, 1902-2376, 1913-2375, 1922-2375, 1927-2439, 1928-2408, 1932-2495, |
| | 1941-2158, 1961-2185, 1963-2244, 1982-2192, 1984-2376, 1985-2452, 1986-2375, 1996-2260, 1999-2252, |
| | 2001-2476, 2004-2480, 2005-2375, 2009-2480, 2023-2482, 2030-2368, 2030-2505, 2031-2482, 2038-2480, |
| | 2041-2186, 2041-2482, 2041-2486, 2056-2482, 2060-2486, 2065-2340, 2068-2497, 2070-2483, 2070-2496, 2080-2482, |
| | 2081-2329, 2085-2480, 2089-2482, 2096-2349, 2096-2482, 2107-2483, 2112-2499, 2120-2361, 2125-2479, |
| | 2131-2486, 2133-2480, 2156-2458, 2158-2353, 2158-2471, 2161-2458, 2163-2427, 2174-2445, 2192-2482, 2201-2479, |
| | 2230-2482, 2256-2458, 2259-2481, 2294-2496, 2367-2480 |
| 75/5635695CB1/ | 1-3634, 285-1033, 458-674, 458-1086, 608-1210, 617-851, 617-885, 617-1187, 775-1193, 802-1063, 827-1308, 827-1483, |
| 4115 | 829-972, 907-1148, 907-1433, 923-1153, 923-1163, 936-1422, 995-1292, 997-1228, 997-1449, 1164-1551, |
| | 1169-1387, 1192-1457, 1215-1667, 1296-1748, 1365-1983, 1417-1966, 1543-2175, 1573-1773, 1620-2171, 1640-1918, |
| | 1646-2224, 1647-1954, 1647-2313, 1902-2375, 1902-2378, 1942-2519, 1947-2649, 2007-2112, 2017-2592, |
| | 2032-2271, 2042-2561, 2048-2652, 2059-2729, 2061-2649, 2073-2683, 2077-2780, 2079-2606, 2144-3046, 2154-2755, |
| | 2161-2429, 2161-2710, 2172-2787, 2187-2404, 2205-3031, 2233-2630, 2236-2914, 2269-2519, 2269-2557, |
| | 2277-2843, 2291-2532, 2320-2629, 2322-2962, 2395-2602, 2398-2695, 2414-3152, 2420-2732, 2440-2710, 2453-2734, |
| | 2459-2746, 2469-2719, 2495-2855, 2515-2843, 2532-3152, 2541-2800, 2577-2852, 2581-2885, 2585-2958, |
| | 2588-3195, 2600-2827, 2618-3172, 2625-2901, 2648-2923, 2678-3274, 2738-3275, 2756-3017, 2771-3018, 2789-2958, |
| | 2798-3039, 2829-3112, 2829-3339, 2846-3098, 2855-3143, 2857-3081, 2876-3106, 2885-3137, |
| | 2896-3354, 2903-3228, 2907-3169, 2945-3198, 2945-3208, 2977-3182, 2977-3293, 2978-3254, 2982-3419, 3076-3346, |
| | 3231-3901, 3374-3623, 3376-4115, 3396-3695, 3405-3966, 3430-3872, 3567-3833, 3607-3886, 3633-3862, |
| | 3633-3980 |
| 76/7503983CB1/ | 1-234, 8-1027, 97-199, 122-655, 122-765, 124-972, 127-692, 129-594, 132-733, 135-717, 136-574, 136-579, 136-588, |
| 1027 | 136-772, 177-411, 177-762, 191-797, 230-755, 230-927, 233-1021, 238-927, 248-927, 249-927, 255-974, 275-548, |
| | 285-772, 287-777, 287-927, 289-559, 300-950, 300-1016, 300-1019, 301-543, 301-547, 301-550, 301-560, 301-565, |
| | 301-568, 301-574, 301-588, 301-594, 301-656, 301-708, 301-871, 301-892, 301-949, 301-953, 301-964, 301-965, |
| | 301-971, 301-973, 301-981, 301-983, 301-1019, 301-1021, 303-514, 303-548, 303-609, 303-1018, 303-1019, |
| | 304-892, 304-971, 304-1020, 305-534, 305-555, 305-558, 305-591, 305-1017, 308-1026, 312-1002, 313-572, 313-613, |
| | 314-1026, 315-549, 316-564, 317-855, 317-927, 319-610, 320-527, 320-591, 321-601, 321-663, 322-563, 322-566, |
| | 324-748, 325-540, 326-1015, 329-848, 330-602, 331-599, 332-465, 333-671, 333-1027, 338-620, 338-855, 341-569, |
| | 341-628, 341-629, 344-981, 344-1027, 346-946, 349-951, 350-589, 351-630, 352-674, 353-876, 355-653, 357-606, |
| | 357-981, 358-645, 359-964, 361-560, 361-591, 363-927, 364-532, 364-609, 365-637, 365-642, 368-620, |
| | 370-605, 370-621, 371-1021, 373-657, 373-925, 373-929, 373-970, 374-950, 375-949, 379-636, 384-843, 384-1021, |
| | 388-592, 391-1021, 392-848, 394-1009, 394-1018, 395-1013, 396-732, 396-971, 399-614, 400-642, 401-1020, 402-954, |
| | 403-648, 404-1027, 405-563, 405-667, 407-982, 408-654, 409-663, 409-674, 409-1026, 411-689, 414-1027, |
| | 415-663, 415-678, 416-650, 417-665, 421-658, 421-673, 421-1021, 421-1027, 422-668, 423-644, 423-1027, 425-830, |
| | 425-959, 426-624, 426-670, 426-672, 433-1027, 434-1013, 442-1006, 443-606, 443-639, 444-627, 444-703, |
| | 444-1027, 445-936, 446-546, 449-969, 452-1027, 455-973, 456-638, 458-1009, 461-993, 469-812, 477-744, 479-1027, |
| | 481-669, 481-1027, 483-1027, 486-1027, 498-994, 498-1027, 499-574, 502-742, 502-1027, 509-733, 511-712, |
| | 511-726, 511-773, 512-725, 514-726, 514-1027, 520-1021, 522-1027, 529-1021, 543-792, 544-1019, 545-1021, 546-1027, |
| | 551-824, 556-779, 556-804, 556-990, 556-1027, 557-770, 557-773, 557-1027, 561-873, 562-830, 565-874, |
| | 565-876, 565-882, 565-1014, 565-1027, 566-1027, 567-776, 567-777, 567-813, 567-830, 567-857, 568-825, 568-832, |
| | 569-857, 570-804, 570-806, 570-848, 570-897, 574-818, 574-964, 574-1019, 575-842, 575-863, |
| | 576-794, 576-821, 576-850, 577-846, 579-796, 579-879, 580-870, 580-1027, 581-1027, 587-1021, 588-821, 588-851, |
| | 588-875, 590-845, 593-832, 594-816, 594-900, 596-810, 596-834, 597-855, 597-1027, 599-878, 600-1021, 601-841, |
| | 601-908, 602-826, 602-847, 602-853, 603-833, 603-886, 603-1027, 604-862, 605-759, 606-855, 606-1021, 607-957, |
| | 607-975, 607-990, 607-1012, 607-1026, 608-973, 608-1027, 609-856, 609-871, 609-884, 610-1015, 611-867, |
| | 612-843, 612-856, 612-857, 612-905, 612-924, 612-991, 612-1021, 612-1027, 613-1021, 614-991, 614-1021, 614-1027, |
| | 615-731, 615-823, 615-914, 616-844, 617-883, 618-848, 618-883, 620-849, 621-857, 621-918, 622-868, 622-1027, |
| | 623-1027, 624-1021, 625-1021, 625-1027, 626-843, 626-962, 626-978, 626-1021, 626-1027, 628-863, 629-878, |
| | 629-991, 631-911, 631-1021, 632-877, 632-1026, 632-1027, 633-981, 633-1021, 634-990, 635-1021, 640-835, |
| | 640-933, 641-1021, 642-862, 642-884, 642-888, 642-903, 642-918, 642-951, 642-1027, 643-900, 644-901, 644-925, |
| | 644-952, 644-981, 645-981, 647-1021, 647-1027, 648-1019, 649-889, 649-905, 649-1004, 652-868, 655-1021, 656-1014, |
| | 657-925, 657-1006, 658-1021, 660-1021, 661-1021, 662-1021, 665-1021, 665-1025, 665-1027, 666-950, |
| | 668-1021, 669-918, 669-935, 669-946, 669-948, 669-982, 669-1021, 669-1025, 670-886, 670-952, 671-970, 672-880, |
| | 673-943, 676-1021, 677-1021, 677-1026, 678-958, 679-960, 680-970, 685-857, 685-1021, 685-1027, 687-1021, |
| | 688-957, 688-981, 688-1018, 691-991, 691-1021, 692-1018, 694-932, 694-1021, 695-1018, 695-1026, 695-1027, |
| | 696-1027, 698-1021, 699-959, 699-984, 700-1026, 701-1025, 704-1021, 708-1027, 710-962, 713-952, 716-933, 718-1021, |
| | 718-1026, 720-1027, 723-967, 723-1019, 724-1019, 724-1021, 725-1027, 726-1021, 730-1021, 733-991, 733-1026, |
| | 735-1001, 735-1011, 735-1016, 736-1021, 741-1021, 742-950, 744-1012, 744-1021, 744-1027, 747-1012, 747-1027, |
| | 748-962, 748-966, 748-977, 748-993, 748-1021, 748-1026, 749-1021, 750-1027, 751-971, 751-1008, 751-1027, |
| | 753-1026, 755-853, 756-1027, 758-914, 758-982, 758-1011, 761-1021, 765-932, 765-978, 765-995, 765-1021, |
| | 767-1027, 768-1021, 774-861, 776-1021, 777-1013, 777-1027, 779-1008, 782-1021, 782-1027, 783-1020, 784-975, |
| | 784-1027, 785-1017, 786-1016, 787-1021, 789-1016, 790-996, 790-1021, 793-1021, 794-1021, 794-1027, 795-1021, |
| | 795-1027, 796-1021, 796-1025, 797-1021, 800-1027, 802-1021, 802-1026, 806-1027, |
| | 808-1021, 820-1019, 821-1027, 826-1012, 830-991, 831-1019, 831-1027, 833-1027, 837-1021, 853-1019, 855-1002, |
| | 855-1027, 860-1027, 861-1021, 861-1027, 864-1027, 873-1018, 873-1020, 873-1027, 874-1018, 874-1027, |
| | 875-1018, 876-1012, 876-1016, 876-1019, 876-1020, 876-1021, 876-1027, 877-1016, 878-1027, 880-1021, 880-1027, |
| | 882-1025, 894-1021, 894-1022, 894-1027, 896-1027, 898-1027, 901-1021, 906-1027, 918-1027, 919-1018, |
| | 919-1027, 941-1021, 949-1027 |
| 77/7503476CB1/ | 1-580, 458-578, 471-577, 486-580, 487-577, 487-580, 488-889, 490-588, 493-580, 494-579, 495-580, 495-634, 499-624, |
| 1103 | 500-629, 506-580, 551-635, 581-635, 581-851, 584-635, 590-634, 590-733, 590-787, 590-939, 593-635, 597-634, |
| | 598-632, 635-870, 635-886, 635-888, 635-894, 636-801, 636-802, 636-819, 636-821, 636-823, 636-834, 636-842, |
| | 636-850, 636-852, 636-853, 636-855, 636-859, 636-862, 636-863, 636-865, 636-869, 636-870, 636-872, 636-873, |
| | 636-874, 636-875, 636-877, 636-878, 636-879, 636-880, 636-881, 636-882, 636-883, 636-885, 636-886, 636-887, |
| | 636-888, 636-889, 636-890, 636-891, 636-892, 636-893, 636-894, 636-895, 636-896, 636-897, 636-898, 636-900, |
| | 636-901, 636-902, 636-903, 636-904, 636-906, 636-908, 636-910, 636-911, 636-914, 636-927, 637-696, 637-890, |
| | 637-892, 637-893, 637-901, 637-916, 637-918, 637-940, 638-881, 638-906, 638-918, 638-926, 639-800, 639-816, |
| | 639-870, 639-874, 639-887, 639-889, 639-898, 640-774, 640-783, 640-870, 640-874, 640-881, 640-884, 640-887, |
| | 640-889, 640-891, 640-893, 640-894, 640-898, 640-899, 640-906, 641-870, 641-890, 641-894, |
| | 642-860, 642-874, 642-880, 642-889, 642-890, 642-891, 642-892, 642-894, 642-901, 642-905, 642-919, 642-935, |
| | 643-873, 643-895, 643-901, 643-914, 643-929, 644-888, 644-919, 645-874, 645-876, 645-891, 645-893, 645-895, |
| | 645-913, 646-890, 646-892, 646-893, 646-894, 647-883, 647-890, 647-891, 648-848, 648-874, 648-887, 648-890, |
| | 648-929, 649-845, 649-867, 649-891, 649-898, 649-902, 650-892, 651-867, 651-882, 651-884, 651-887, 651-889, |
| | 651-891, 651-909, 651-912, 652-758, 652-880, 652-890, 652-894, 652-917, 653-838, 653-891, 653-900, 653-901, |
| | 653-902, 654-890, 654-891, 654-893, 654-904, 654-906, 655-902, 655-904, 656-869, 656-874, 656-896, 656-900, |
| | 656-917, 656-921, 657-894, 657-908, 658-795, 658-859, 658-870, 658-874, 658-890, 658-891, 658-892, 658-900, |
| | 658-901, 658-905, 658-916, 658-918, 659-887, 659-889, 659-891, 660-829, 660-838, 660-884, 660-888, 660-893, |
| | 661-879, 661-891, 661-894, 661-895, 661-908, 661-915, 662-890, 662-895, 663-870, 663-893, 663-896, 663-898, |
| | 663-906, 664-872, 664-914, 664-919, 664-923, 665-898, 665-904, 666-873, 666-874, 666-885, 666-887, 666-889, |
| | 666-890, 666-891, 666-895, 667-890, 667-897, 668-873, 668-905, 668-910, 668-919, |
| | 669-875, 669-883, 669-889, 669-893, 669-901, 669-912, 669-914, 670-890, 670-894, 670-907, 671-865, 671-874, |
| | 671-889, 671-908, 672-901, 672-905, 672-907, 672-911, 672-936, 673-864, 673-870, 673-874, 673-878, 673-928, |
| | 673-929, 674-870, 674-882, 674-890, 674-903, 675-891, 675-899, 675-909, 676-887, 676-906, 677-874, 677-910, |
| | 677-921, 678-899, 680-894, 680-897, 680-899, 680-900, 680-905, 681-870, 681-873, 681-894, 681-920, 682-890, |
| | 682-891, 682-903, 683-874, 683-902, 684-784, 684-878, 684-890, 684-903, 684-921, 685-811, 685-893, 685-904, |
| | 686-890, 686-892, 686-893, 687-890, 687-893, 687-895, 687-909, 689-883, 689-884, 689-892, 689-902, 690-874, |
| | 690-900, 691-870, 691-920, 692-874, 692-894, 692-910, 692-923, 693-896, 693-909, 694-939, 695-891, 696-912, |
| | 696-940, 697-888, 697-905, 698-816, 699-861, 699-888, 699-889, 699-899, 699-901, 699-911, 699-961, 700-874, |
| | 700-889, 701-848, 701-874, 701-883, 701-906, 702-887, 702-890, 702-900, 703-874, 703-889, 703-902, 703-912, |
| | 703-915, 703-925, 705-882, 705-889, 705-895, 706-874, 706-898, 706-909, 706-940, 707-909, 708-892, 709-911, |
| | 709-938, 711-874, 711-882, 711-890, 714-867, 715-740, 715-870, 715-874, 715-904, 715-929, |
| | 716-874, 716-902, 716-931, 718-874, 718-890, 719-870, 721-915, 723-942, 725-839, 725-864, 725-928, 726-901, |
| | 727-870, 727-891, 728-891, 728-893, 729-841, 729-865, 729-874, 729-899, 729-901, 729-905, 729-911, 729-972, |
| | 730-891, 730-951, 731-890, 731-905, 731-984, 732-874, 732-886, 733-874, 734-854, 734-856, 734-900, 734-1045, |
| | 742-879, 744-896, 746-825, 748-926, 751-917, 752-885, 758-888, 758-900, 758-913, 760-892, 767-889, 767-899, |
| | 769-888, 770-905, 774-893, 782-880, 786-893, 792-870, 792-914, 795-888, 795-896, 796-955, 796-1103, 805-874, |
| | 816-964 |
| 78/7504023CB1/ | 1-203, 1-206, 1-815, 7-116, 10-304, 15-149, 24-201, 25-178, 30-122, 81-229, 232-428, 232-463, 249-461, 252-738, |
| 822 | 253-597, 263-538, 278-406, 297-517, 297-544, 297-559, 300-753, 300-778, 300-815, 305-756, 305-822, 337-612, |
| | 349-804, 352-642, 353-629, 357-629, 381-809, 387-629, 398-822, 401-629, 406-629, 407-629, 409-629, 410-629, |
| | 421-629, 426-629, 441-629, 441-808, 441-822, 442-629, 444-646, 447-690, 447-716, 448-693, 449-629, 449-697, |
| | 452-730, 456-680, 456-700, 456-791, 457-783, 461-629, 463-698, 463-701, 466-629, 472-629, 476-629, 479-629, |
| | 480-608, 482-629, 488-518, 489-806, 489-822, 492-629, 492-723, 493-706, 494-695, 497-625, 497-629, 502-629, |
| | 504-709, 509-757, 510-629, 511-704, 516-666, 517-767, 519-710, 520-629, 521-629, 524-629, 524-658, 524-660, |
| | 524-813, 526-822, 527-641, 527-646, 538-822, 540-629, 540-790, 546-629, 547-822, 549-809, 552-629, 558-739, |
| | 559-822, 563-629, 576-809, 577-809, 634-822, 680-809, 722-809 |
| 79/7504128CB1/ | 1-592, 12-189, 23-106, 23-126, 23-131, 23-135, 23-139, 23-141, 23-158, 23-166, 23-171, 23-176, 23-201, 23-575, |
| 877 | 24-103, 24-109, 24-112, 24-116, 24-124, 24-132, 24-135, 24-180, 24-181, 24-182, 24-190, 25-116, 25-127, 25-142, |
| | 25-144, 25-169, 25-180, 27-118, 27-135, 27-140, 27-167, 27-173, 28-171, 28-195, 29-153, 29-174, 29-187, 29-202, |
| | 29-290, 31-162, 32-90, 36-279, 37-192, 38-293, 111-173, 170-575, 203-576, 231-576, 235-532, 249-583, 249-596, |
| | 253-596, 256-573, 299-594, 308-559, 320-565, 320-621, 337-630, 339-598, 339-645, 345-607, 347-581, 356-619, |
| | 363-617, 368-591, 368-877, 372-575, 372-626, 373-580, 376-584, 376-603, 376-636, 383-628, 389-576, 390-582, |
| | 405-576, 424-590, 433-521, 434-563, 434-576, 435-589, 443-627, 461-576, 462-597, 462-610, 471-591, 472-576, |
| | 480-582, 481-669, 504-575 |
| 80/4529338CB1 | 1-1085, 41-64, 65-238, 65-277, 65-317, 65-351, 65-370, 65-408, 65-425, 65-430, 65-474, 65-475, 65-479, 65-545, |
| 1306 | 65-647, 65-721, 67-527, 67-699, 79-547, 86-197, 90-521, 93-662, 93-704, 111-509, 114-676, 124-702, 140-864, 160-892, |
| | 187-429, 187-615, 187-650, 187-659, 187-696, 187-761, 219-861, 223-1093, 224-716, 258-503, 259-980, 376-1186, |
| | 381-644, 382-1096, 383-931, 397-1042, 419-1094, 434-1049, 434-1306, 438-946, 442-1072, 463-1118, 467-1104, |
| | 470-935, 477-705, 479-1266, 483-1036, 497-524, 513-1132, 520-1041, 523-1185, 539-1110, 540-1091, 547-1047, |
| | 550-1049, 552-1063, 553-1128, 561-837, 566-1010, 579-1178, 596-1177, 600-1128, 614-1226, 620-1212, 625-1125, |
| | 635-1156, 647-1222, 647-1267, 657-956, 692-1233, 840-1144, 898-1132 |
| 81/7503460CB1/ | 1-355, 1-479, 1-844, 3-257, 15-324, 16-317, 16-468, 20-396, 20-763, 20-768, 25-288, 26-648, 33-300, 33-316, 35-345, |
| 1016 | 43-391, 44-496, 44-505, 49-505, 50-316, 51-298, 55-221, 55-310, 60-496, 61-410, 72-303, 102-770, 103-768, |
| | 148-761, 166-429, 168-410, 175-437, 196-405, 511-745, 522-1016, 523-813, 535-829, 540-832, 543-843, 546-836, |
| | 587-794, 606-846, 760-843 |
| 82/5466630CB1/ | 1-557, 22-510, 98-761, 106-562, 520-968, 552-1171, 566-1171, 620-1094, 620-1283, 620-1288, 620-1308, 620-1321, |
| 4204 | 635-1233, 824-1245, 881-1696, 892-1316, 892-1553, 912-1347, 926-1192, 926-1201, 930-1203, 930-1423, |
| | 1201-1687, 1214-1602, 1228-1556, 1279-2034, 1284-1589, 1348-1762, 1350-1761, 1350-1762, 1440-2105, 1453-2154, |
| | 1496-2107, 1618-2321, 1646-2386, 1650-2283, 1719-1981, 1729-2438, 1751-2435, 1751-2529, 1791-1888, |
| | 1850-2380, 1920-2679, 1935-2675, 2005-2694, 2111-2618, 2113-2510, 2140-2550, 2156-2387, 2164-2550, 2208-2550, |
| | 2234-2400, 2494-3078, 2534-3169, 2539-3354, 2543-3360, 2566-3161, 2662-3420, 2669-3277, 2726-3291, |
| | 2754-3412, 2793-3344, 2830-3035, 2832-3091, 2832-3323, 2842-3477, 2860-3379, 2860-3426, 2868-3398, 2875-3477, |
| | 2899-3181, 2899-3328, 2899-3339, 2899-3454, 2899-3479, 2899-3480, 2899-3482, 2899-3508, 2899-3549, |
| | 2899-3550, 2899-3632, 2899-3712, 2899-3714, 2899-3721, 2899-3744, 2899-3749, 2903-3176, 2903-3528, 2925-3670, |
| | 2934-3511, 2937-3678, 2956-3448, 2958-3603, 2968-3454, 2983-3215, 3001-3381, 3012-3887, |
| | 3013-3567, 3019-3777, 3024-3606, 3027-3263, 3027-3576, 3032-3595, 3035-3693, 3046-3668, 3112-3525, 3113-3920, |
| | 3123-3534, 3123-3546, 3140-3798, 3149-3688, 3151-3800, 3162-3920, 3198-3853, 3202-3836, 3205-3915, |
| | 3206-3824, 3218-3788, 3229-3779, 3230-3754, 3234-3885, 3236-3904, 3239-3882, 3252-3716, 3272-3943, 3276-3793, |
| | 3278-3793, 3284-3364, 3284-3720, 3293-3782, 3295-3617, 3302-3563, 3305-3850, 3306-3983, 3311-3878, |
| | 3313-3867, 3324-3937, 3329-3895, 3335-3885, 3340-3953, 3341-3885, 3343-3925, 3345-3811, 3350-3911, 3354-3726, |
| | 3355-3980, 3357-3861, 3358-4026, 3372-3988, 3395-3810, 3398-3810, 3400-3881, 3414-3689, 3424-3825, |
| | 3427-3926, 3428-4016, 3434-4015, 3436-3927, 3437-3825, 3438-4028, 3458-4021, 3471-3736, 3489-3975, 3489-4103, |
| | 3504-3738, 3511-4204 |
| 83/7503474CB1/ | 1-272, 1-1410, 11-199, 20-155, 20-213, 24-213, 62-213, 244-541, 251-549, 260-516, 282-1047, 293-654, 301-524, |
| 2211 | 306-939, 315-1043, 337-880, 379-684, 394-878, 464-612, 470-740, 476-839, 479-762, 480-612, 496-701, 530-779, |
| | 530-1024, 558-1257, 579-689, 586-950, 589-1362, 605-834, 605-1189, 641-1327, 655-886, 680-932, 721-1337, 733-1354, |
| | 734-982, 741-762, 743-1398, 759-1005, 779-1015, 785-1347, 832-1125, 882-1117, 882-1139, 926-1131, 944-1191, |
| | 945-1184, 945-1222, 946-1186, 958-1409, 960-1409, 963-1407, 966-1205, 975-1412, 978-1408, 996-1254, |
| | 998-1262, 1028-1409, 1036-1204, 1041-1237, 1055-1330, 1055-1409, 1058-1409, 1069-1414, 1072-1413, 1104-1334, |
| | 1106-1405, 1115-1461, 1122-1409, 1123-1412, 1126-1413, 1132-1410, 1134-1409, 1139-1409, 1168-1409, |
| | 1170-1423, 1179-1409, 1235-2211, 1247-1407, 1328-1413 |
| 84/7503498CB1/ | 1-196, 8-219, 8-228, 8-234, 8-236, 8-247, 8-264, 8-265, 8-338, 8-390, 8-421, 8-440, 8-469, 8-481, 8-576, 8-582, 8-1060, |
| 1076 | 8-1074, 9-260, 13-255, 280-588, 300-546, 323-778, 340-678, 388-1008, 404-914, 406-910, 411-722, 415-1037, |
| | 418-768, 425-657, 426-641, 426-1070, 428-824, 430-717, 435-1036, 439-1045, 478-1035, 501-741, 511-722, |
| | 517-757, 522-770, 522-1076, 530-1015, 536-1076, 541-812, 545-788, 548-1009, 579-913, 581-937, 590-1052, 598-1058, |
| | 607-1059, 615-1057, 634-1059, 640-1060, 642-1059, 643-1065, 661-1037, 688-1059, 688-1060, 691-1025, |
| | 693-1057, 708-1054, 715-1022, 718-1076, 760-1044, 761-1059, 785-1058, 788-1058, 838-1064, 840-1069, 865-1057, |
| | 939-1076, 947-1076, |
| 85/7504119CB1/ | 1-252, 1-298, 7-263, 7-268, 7-282, 7-1112, 15-219, 17-528, 20-266, 20-510, 23-321, 24-272, 24-284, 25-585, 26-300, |
| 1118 | 26-315, 27-274, 28-288, 28-518, 30-321, 31-566, 32-297, 32-360, 32-575, 32-608, 32-686, 34-298, 34-314, 34-335, |
| | 37-605, 38-283, 38-297, 39-301, 47-679, 49-684, 51-471, 61-354, 62-310, 94-643, 118-638, 167-731, 203-444, |
| | 208-830, 280-376, 320-867, 365-513, 365-867, 440-703, 595-867, 701-1108, 704-1111, 706-1106, 744-1113, 864-1113, |
| | 865-1113, 884-1104, 889-1116, 915-1114, 948-1118, |
| 86/71532805CB1/ | 1-174, 1-435, 1-477, 1-486, 1-492, 1-513, 1-613, 1-618, 1-630, 4-141, 51-393, 58-197, 259-808, 280-958, 362-882, |
| 986 | 434-986, |
| 87/5502992CB1/ | 1-722, 27-404, 157-488, 282-3498, 294-623, 318-582, 318-689, 369-1015, 405-552, 775-1367, 1013-1369, 1502-2027, |
| 6546 | 1503-2136, 1553-2192, 1602-2017, 1612-1857, 2924-3703, 2931-3567, 2978-3598, 3019-3452, 3024-3479, |
| | 3024-3525, 3024-3626, 3093-3699, 3122-3619, 3169-3783, 3179-3775, 3194-3703, 3198-3619, 3202-3605, 3208-3496, |
| | 3250-3759, 3266-3579, 3266-3759, 3304-3759, 3328-3759, 3342-3759, 3353-3986, 3362-3642, 3364-3945, |
| | 3364-3979, 3377-3910, 3384-3928, 3392-3774, 3409-4265, 3419-4117, 3433-4153, 3443-3925, 3454-3722, 3454-3916, |
| | 3491-4274, 3601-4152, 3626-4159, 3640-4365, 3641-4352, 3646-4219, 3670-4272, 3683-4199, 3696-4289, |
| | 3713-4308, 3738-4245, 3748-4270, 3765-4478, 3765-4555, 3767-4336, 3768-4329, 3778-4524, 3780-4397, 3799-4473, |
| | 3801-4416, 3802-4368, 3804-4366, 3812-4565, 3816-4467, 3818-4366, 3818-4470, 3840-4538, 3880-4359, |
| | 3903-4209, 3906-4672, 3925-4575, 3965-4330, 3968-4464, 3970-4289, 3979-4548, 3993-4360, 3996-4251, 4001-4631, |
| | 4018-4639, 4033-4432, 4035-4859, 4040-4484, 4053-4738, 4056-4432, 4062-4766, 4073-4320, |
| | 4095-4584, 4098-4337, 4100-4524, 4111-4520, 4115-4450, 4124-4820, 4213-4901, 4234-4871, 4260-5113, 4265-4924, |
| | 4338-5035, 4397-5018, 4407-5003, 4431-5026, 4459-5049, 4477-5115, 4533-5185, 4575-5350, 4584-5377, |
| | 4590-5211, 4591-5445, 4600-5208, 4603-5307, 4609-5164, 4633-5264, 4636-5129, 4641-5454, 4646-5217, 4646-5314, |
| | 4649-5148, 4657-5478, 4664-5016, 4673-4863, 4673-4871, 4680-5340, 4691-5202, 4694-5275, 4701-5259, |
| | 4705-5366, 4715-4966, 4723-4983, 4723-5461, 4750-5358, 4752-5455, 4759-5180, 4770-5027, 4786-5023, 4787-5020, |
| | 4787-5190, 4787-5229, 4787-5262, 4787-5272, 4787-5404, 4787-5543, 4789-5384, 4791-5596, 4803-5253, |
| | 4813-5060, 4821-5339, 4828-5545, 4831-5547, 4837-5474, 4838-5309, 4847-5404, 4922-5568, 4928-5551, 4936-5580, |
| | 4941-5720, 4950-5680, 4966-5492, 4982-5485, 4996-5542, 5006-5451, 5006-5758, 5022-5569, 5031-5484, |
| | 5032-5633, 5054-5484, 5064-5650, 5091-5484, 5093-5484, 5093-5485, 5103-5620, 5106-5850, 5109-5587, 5120-5484, |
| | 5122-5331, 5123-5772, 5142-5713, 5155-5415, 5156-5510, 5160-5431, 5162-5838, 5182-5488, 5186-5711, |
| | 5189-5552, 5194-5846, 5203-5452, 5214-5265, 5234-5861, 5238-5489, 5238-5781, 5240-5539, |
| | 5242-5485, 5242-5789, 5243-5799, 5244-5521, 5249-5505, 5250-5496, 5257-5671, 5258-5819, 5264-5445, 5266-5635, |
| | 5290-5671, 5290-5738, 5293-5827, 5306-5602, 5312-5566, 5313-5385, 5313-5512, 5313-5556, 5325-5607, |
| | 5329-6088, 5348-5633, 5348-5666, 5359-5626, 5361-5930, 5367-5598, 5385-5909, 5387-5858, 5412-5857, 5443-5883, |
| | 5445-5837, 5460-5980, 5460-5989, 5482-5919, 5488-6009, 5488-6033, 5495-6024, 5507-6224, 5510-6043, |
| | 5515-6224, 5521-5856, 5532-6058, 5540-5920, 5540-6091, 5546-5833, 5547-6091, 5551-6117, 5555-5821, 5562-5819, |
| | 5573-6145, 5576-5875, 5588-6047, 5589-6038, 5603-6038, 5605-6091, 5608-5897, 5616-6090, 5624-5994, |
| | 5642-6546, 5660-5943, 5666-6220, 5670-6131, 5672-5916, 5672-5923, 5672-6047, 5672-6128, 5672-6139, 5674-6204, |
| | 5683-5927, 5683-6254, 5690-6247, 5690-6253, 5719-6303, 5738-6037, 5739-5909, 5739-6022, 5739-6038, |
| | 5739-6047, 5739-6090, 5739-6091, 5739-6140, 5742-5967, 5756-5975, 5789-6242, 5794-6253, 5796-6253, 5806-6080, |
| 88/7503828CB1/ | 1-253, 1-276, 1-379, 1-383, 1-3724, 8-307, 34-592, 44-641, 46-558, 51-321, 52-352, 54-565, 54-658, 57-305, 58-685, |
| 3737 | 58-818, 59-339, 59-606, 60-942, 66-667, 66-903, 67-804, 68-307, 68-566, 68-572, 68-626, 68-640, 68-731, 68-854, |
| | 69-315, 69-325, 69-342, 69-351, 69-363, 69-369, 69-576, 69-586, 69-642, 69-701, 69-858, 70-362, 72-294, 74-338, |
| | 74-672, 76-738, 80-651, 81-592, 83-257, 83-747, 86-396, 87-377, 87-683, 113-712, 114-775, 118-670, 137-379, |
| | 163-621, 195-733, 197-489, 230-777, 246-954, 249-953, 252-429, 285-929, 285-1068, 293-1009, 324-998, 335-926, |
| | 337-631, 365-737, 378-545, 385-874, 411-661, 411-1019, 434-873, 455-730, 509-947, 521-993, 522-993, 522-998, |
| | 526-1009, 541-1000, 580-993, 625-1267, 627-883, 658-1235, 678-828, 679-1259, 683-1205, 728-997, 737-989, |
| | 780-1489, 794-1032, 932-1444, 938-1553, 943-1617, 967-1668, 973-1377, 987-1155, 1011-1535, 1021-1257, 1026-1614, |
| | 1026-1617, 1044-1617, 1045-1456, 1073-1644, 1074-1684, 1124-1388, 1169-1710, 1173-1408, 1179-1453, |
| | 1217-1510, 1226-1734, 1240-1723, 1273-1715, 1279-1774, 1316-1751, 1316-1904, 1363-1662, |
| | 1488-1715, 1558-1813, 1571-2169, 1600-1858, 1605-1752, 1618-2172, 1618-2174, 1625-1784, 1625-1808, 1625-1897, |
| | 1625-1919, 1625-2040, 1625-2046, 1625-2057, 1625-2058, 1625-2062, 1625-2063, 1625-2157, 1625-2160, |
| | 1625-2163, 1625-2205, 1625-2278, 1625-2289, 1625-2301, 1625-2319, 1625-2323, 1625-2339, 1627-2134, 1627-2167, |
| | 1725-2417, 1758-2155, 1759-2259, 1762-2329, 1766-2040, 1774-2036, 1781-2034, 1793-2080, 1799-2231, |
| | 1801-2078, 1824-2077, 1824-2354, 1832-2083, 1832-2410, 1833-2392, 1845-2361, 1873-2270, 1922-2175, 1928-2255, |
| | 1933-2235, 1940-2168, 1942-2444, 1953-2206, 1955-2159, 1955-2474, 1973-2177, 1973-2178, 2051-2457, |
| | 2067-2719, 2072-2681, 2101-2359, 2102-2342, 2124-2245, 2124-2336, 2124-2367, 2137-2848, 2155-2265, 2160-2443, |
| | 2188-2423, 2213-2823, 2222-2530, 2228-2507, 2246-2413, 2285-3087, 2286-2537, 2297-2547, 2334-2458, |
| | 2346-2968, 2348-2660, 2375-2734, 2391-2683, 2392-2724, 2400-2562, 2460-2740, 2464-3078, 2465-2703, 2468-3087, |
| | 2472-3054, 2475-2750, 2479-2854, 2503-2757, 2507-2947, 2534-3189, 2554-2783, 2567-2807, 2592-3219, |
| | 2615-2867, 2629-2871, 2631-3200, 2637-2934, 2654-2842, 2661-2908, 2670-2782, 2671-2837, 2672-3188, |
| | 2675-2966, 2678-2869, 2678-2953, 2680-2910, 2683-2872, 2684-2984, 2695-2862, 2696-2871, 2705-2932, 2711-3014, |
| | 2715-2871, 2717-2955, 2717-2986, 2724-3187, 2731-2951, 2731-2965, 2734-2922, 2742-3393, 2746-3045, |
| | 2761-2922, 2781-3288, 2787-3072, 2824-3425, 2850-3429, 2856-3662, 2874-3102, 2878-3147, 2880-3036, 2952-3239, |
| | 2959-3636, 2990-3263, 2991-3709, 3002-3288, 3018-3288, 3020-3279, 3020-3280, 3037-3128, 3037-3368, |
| | 3058-3636, 3058-3723, 3104-3288, 3116-3382, 3134-3426, 3142-3709, 3152-3709, 3169-3393, 3172-3689, 3208-3724, |
| | 3226-3415, 3241-3533, 3243-3490, 3250-3709, 3251-3725, 3263-3546, 3279-3709, 3288-3630, 3288-3708, |
| | 3288-3709, 3288-3710, 3288-3712, 3288-3727, 3290-3589, 3290-3669, 3290-3693, 3290-3727, 3294-3708, 3297-3727, |
| | 3300-3709, 3301-3693, 3310-3706, 3314-3712, 3316-3709, 3317-3712, 3318-3737, 3324-3631, 3325-3656, |
| | 3328-3706, 3333-3628, 3343-3709, 3352-3711, 3352-3722, 3359-3709, 3363-3712, 3368-3711, 3368-3713, 3371-3709, |
| | 3386-3709, 3390-3712, 3391-3706, 3391-3708, 3391-3709, 3392-3713, 3399-3678, 3410-3711, 3415-3666, |
| | 3426-3727, 3432-3712, 3434-3723, 3440-3724, 3442-3709, 3457-3647, 3458-3724, 3463-3699, 3479-3727, |
| | 3484-3717, 3488-3726, 3495-3709, 3505-3705, 3525-3709, 3525-3726, 3544-3725, 3552-3697, 3552-3707, 3552-3708, |
| | 3557-3713, 3558-3702, 3569-3712, 3615-3724, 3639-3708 |
| 89/2647325CB1/ | 1-457, 241-927, 289-785, 290-745, 304-767, 306-775, 315-767, 395-623, 395-678, 395-694, 395-742, 395-751, 395-785, |
| 2171 | 395-788, 456-790, 462-792, 478-775, 524-895, 525-1239, 553-780, 584-1464, 587-793, 590-775, 737-1421, |
| | 870-1236, 879-1161, 882-1361, 900-1237, 943-1158, 1038-1250, 1093-1348, 1093-1602, 1166-1997, 1201-1840, |
| | 1248-1565, 1254-1769, 1332-1541, 1348-1958, 1359-1613, 1359-1615, 1362-1824, 1362-1877, 1362-1981, 1382-1618, |
| | 1403-1997, 1476-1588, 1488-2132, 1548-1821, 1571-2143, 1585-2132, 1592-2148, 1614-2108, 1629-2149, |
| | 1630-1896, 1632-2086, 1652-1872, 1652-2162, 1658-2151, 1737-1996, 1743-2002, 1756-2009, 1764-2059, 1775-1947, |
| | 1775-2047, 1785-2066, 1796-2103, 1814-2108, 1858-2096, 1871-2134, 1881-2171 |
| 90/7495416CB1/ | 1-817, 2-242, 229-577, 330-569, 335-569, 336-508, 346-900, 358-1008, 410-1039, 410-1043, 555-582, 555-667, 555-697, |
| 1565 | 555-715, 555-835, 589-656, 589-667, 597-656, 681-740, 698-736, 698-751, 698-805, 698-883, 698-959, 701-739, |
| | 703-889, 703-992, 703-994, 758-1490, 773-814, 773-915, 774-850, 780-1166, 787-829, 787-915, 857-906, 857-913, |
| | 857-1000, 858-987, 859-1075, 871-1057, 871-1162, 891-1051, 891-1171, 950-1135, 950-1211, 955-1162, 995-1156, |
| | 1027-1162, 1093-1171, 1103-1171, 1157-1502, 1157-1512, 1157-1514, 1157-1536, 1158-1491, 1158-1514, |
| | 1169-1549, 1176-1517, 1194-1431, 1194-1504, 1194-1513, 1194-1515, 1194-1526, 1194-1529, 1194-1531, 1194-1532, |
| | 1194-1533, 1194-1534, 1194-1548, 1194-1549, 1197-1549, 1207-1548, 1223-1548, 1241-1516, 1243-1514, |
| | 1330-1565, 1352-1565, 1407-1548 |
| 91/8096177CB1/ | 1-890, 31-775, 35-589, 318-1042, 319-1071, 438-863, 576-906, 631-1221, 631-1274, 673-1247, 689-1149, 849-1569, |
| 5121 | 1075-1598, 1076-1342, 1095-1705, 1112-1783, 1170-1610, 1176-1517, 1176-1793, 1227-1495, 1227-1711, |
| | 1334-1960, 1356-1892, 1393-1665, 1475-1892, 1510-1770, 1511-2179, 1512-2111, 1520-2257, 1556-1734, 1587-2232, |
| | 1594-2197, 1651-1794, 1651-1804, 1651-1808, 1651-1868, 1651-1876, 1651-1984, 1651-2027, 1651-2070, |
| | 1651-2113, 1651-2148, 1651-2149, 1651-2182, 1651-2195, 1651-2200, 1676-1749, 1676-1771, 1676-1808, 1676-1813, |
| | 1676-1879, 1676-1943, 1676-1944, 1676-1945, 1676-1948, 1676-1975, 1676-2027, 1676-2028, 1676-2033, |
| | 1676-2148, 1677-1774, 1677-1807, 1677-1814, 1677-1821, 1678-1952, 1683-1813, 1683-2033, 1686-1757, 1686-1784, |
| | 1686-1792, 1686-1904, 1686-1943, 1686-2003, 1686-2029, 1686-2037, 1686-2052, 1686-2056, 1686-2117, |
| | 1689-2117, 1696-1925, 1699-2029, 1699-2033, 1700-2267, 1700-2296, 1710-2349, 1712-1821, 1712-1948, 1713-1948, |
| | 1714-1800, 1714-1821, 1714-2117, 1715-2033, 1716-2033, 1718-1821, 1724-2056, |
| | 1735-1877, 1735-1976, 1735-2032, 1735-2154, 1735-2195, 1735-2263, 1735-2280, 1735-2284, 1741-2037, 1743-2056, |
| | 1755-1984, 1755-2033, 1755-2149, 1755-2196, 1755-2197, 1755-2200, 1757-2232, 1760-2284, 1766-2149, |
| | 1767-2032, 1773-2200, 1780-2117, 1780-2359, 1783-1963, 1787-2276, 1787-2284, 1797-2387, 1799-2117, 1816-2224, |
| | 1819-2072, 1819-2240, 1819-2290, 1819-2388, 1819-2402, 1819-2412, 1826-2280, 1839-2033, 1839-2117, |
| | 1839-2200, 1839-2284, 1841-2284, 1846-2033, 1854-1933, 1854-1938, 1854-1961, 1854-2060, 1854-2116, 1854-2123, |
| | 1854-2205, 1854-2224, 1854-2285, 1855-2200, 1857-2506, 1866-2200, 1872-2473, 1876-2551, 1877-2361, |
| | 1881-2224, 1882-2033, 1882-2037, 1882-2093, 1882-2117, 1882-2224, 1882-2285, 1897-2056, 1909-2457, 1911-2467, |
| | 1913-2200, 1914-2033, 1916-2200, 1923-2154, 1923-2280, 1923-2284, 1925-2285, 1927-2033, 1928-2022, |
| | 1928-2032, 1928-2065, 1928-2144, 1928-2209, 1928-2285, 1935-2033, 1935-2203, 1948-2376, |
| | 1950-2266, 1950-2284, 1951-2150, 1955-2033, 1955-2285, 1959-2033, 1963-2200, 1964-2519, 1968-2568, 1997-2200, |
| | 1997-2274, 1997-2284, 1997-2285, 2000-2502, 2007-2205, 2007-2227, 2007-2285, 2009-2285, 2012-2101, |
| | 2012-2106, 2012-2123, 2012-2215, 2012-2284, 2017-2233, 2019-2284, 2041-2224, 2042-2284, 2042-2285, 2043-2735, |
| | 2047-2454, 2048-2215, 2048-2284, 2050-2224, 2050-2284, 2050-2285, 2050-2773, 2051-2284, 2081-2284, |
| | 2087-2284, 2091-2285, 2097-2152, 2097-2190, 2097-2213, 2097-2241, 2101-2241, 2115-2285, 2127-2284, 2127-2285, |
| | 2132-2241, 2132-2634, 2134-2285, 2166-2227, 2166-2284, 2179-2660, 2179-2679, 2185-2276, 2185-2807, |
| | 2200-2285, 2207-2505, 2211-2453, 2212-2986, 2216-2459, 2307-2511, 2307-2512, 2307-2668, 2307-2933, 2408-3012, |
| | 2412-2929, 2439-2709, 2439-3072, 2475-3062, 2488-3061, 2489-3062, 2492-3062, 2503-3048, 2554-3062, |
| | 2586-3062, 2587-3279, 2613-3062, 2615-2875, 2643-3209, 2731-3019, 2776-3160, 2800-3325, 2823-3448, 2867-3685, |
| | 2929-3071, 2929-3073, 2953-3575, 2957-3601, 3011-3275, 3049-3337, 3049-3563, 3049-3701, 3082-3520, |
| | 3082-3700, 3092-3471, 3110-3598, 3132-3362, 3132-3510, 3133-3387, 3239-3540, |
| | 3247-3491, 3247-3827, 3247-3833, 3262-3492, 3262-3782, 3263-3526, 3287-3824, 3316-3562, 3316-3979, 3319-3868, |
| | 3328-3945, 3328-3970, 3341-3895, 3354-3929, 3382-4009, 3383-3587, 3430-3585, 3445-3842, 3476-3753, |
| | 3476-3960, 3508-3705, 3522-3780, 3547-3929, 3644-3769, 3725-3996, 3748-4308, 3836-4451, 3933-4180, 4017-4674, |
| | 4227-4893, 4235-4508, 4235-4523, 4235-4530, 4275-4922, 4276-5100, 4298-4812, 4356-4874, 4379-4657, |
| | 4435-5121, 4479-4944, 4490-4946, 4499-4944, 4499-4948, 4592-5078, 4606-5069, 4607-4936, 4640-4944, 4663-4951, |
| | 4680-5068 |
| 92/666763CB1/ | 1-385, 1-427, 1-429, 320-696, 376-785, 640-1183, 640-1344, 647-774, 647-1320, 885-1550, 904-1438, 904-2626, |
| 2626 | 908-1593, 914-1205, 945-1374, 961-1113, 964-1365, 1276-1400, 1354-2029, 1575-2053, 1663-2053, 1677-1906, |
| | 1733-1937, 1733-2397, 1733-2439, 2150-2406, 2194-2626, 2329-2556, 2450-2626 |
| 93/7504091CB1/ | 1-72, 1-82, 1-96, 1-100, 1-119, 1-206, 1-212, 1-216, 1-219, 1-223, 1-227, 1-230, 1-231, 1-234, 1-237, 1-241, 1-243, |
| 1620 | 1-244, 1-252, 1-253, 1-255, 1-257, 1-258, 1-264, 1-279, 1-280, 1-288, 1-293, 1-304, 1-305, 1-306, 1-317, 1-330, 1-398, |
| | 1-502, 1-1583, 2-229, 2-248, 2-249, 4-267, 6-311, 7-258, 10-239, 12-261, 12-561, 13-293, 13-587, 18-304, 18-442, |
| | 21-241, 22-264, 22-302, 22-320, 22-466, 23-308, 24-265, 24-316, 29-307, 36-274, 38-270, 40-283, 43-310, 45-373, |
| | 45-444, 46-309, 47-271, 56-298, 68-299, 90-415, 94-304, 94-481, 123-390, 123-394, 129-371, 130-382, 133-845, |
| | 134-376, 149-401, 150-421, 156-337, 156-464, 186-452, 186-472, 186-486, 212-845, 220-499, 224-502, 260-473, |
| | 277-502, 288-502, 317-482, 376-488, 503-746, 508-1140, 512-777, 513-741, 520-794, 520-810, 530-787, 531-1019, |
| | 540-812, 545-803, 545-893, 547-1001, 553-948, 569-882, 570-1142, 571-1247, 574-1143, 596-852, 599-1226, |
| | 600-872, 601-831, 603-1190, 606-860, 606-1143, 606-1263, 609-1146, 616-1197, 630-915, 635-1349, 643-910, 643-932, |
| | 656-936, 656-1122, 657-910, 658-887, 660-1208, 664-1082, 664-1090, 664-1153, |
| | 675-799, 675-806, 687-913, 690-966, 690-1005, 691-950, 695-1354, 703-1170, 706-1048, 709-994, 723-991, 724-1383, |
| | 735-1014, 738-1169, 747-1000, 760-1220, 767-1050, 771-1046, 777-1352, 781-1010, 790-1283, 790-1547, |
| | 792-1075, 797-1049, 797-1424, 802-1418, 803-1284, 831-1257, 837-1132, 842-1141, 843-1284, 845-1166, 845-1387, |
| | 848-981, 852-1145, 859-1116, 874-1014, 878-1284, 881-1283, 882-1283, 884-1278, 884-1289, 885-1282, 887-1282, |
| | 895-1181, 897-1120, 900-1282, 903-1235, 903-1377, 903-1578, 905-1170, 905-1402, 906-1222, 910-1282, |
| | 911-1265, 911-1282, 922-1284, 925-1174, 927-1412, 929-1173, 929-1250, 931-1424, 932-1155, 932-1184, 932-1201, |
| | 934-1171, 940-1168, 942-1423, 945-1104, 956-1262, 964-1423, 965-1428, 966-1283, 966-1427, 967-1172, |
| | 968-1284, 972-1427, 974-1426, 974-1430, 976-1281, 979-1278, 981-1430, 983-1421, 988-1429, 989-1426, 989-1427, |
| | 993-1424, 994-1226, 995-1108, 1001-1272, 1002-1251, 1005-1430, 1008-1412, 1010-1430, 1012-1429, 1019-1260, |
| | 1020-1254, 1020-1543, 1026-1282, 1029-1293, 1029-1423, 1029-1427, 1031-1429, 1034-1319, |
| | 1036-1240, 1036-1287, 1036-1426, 1036-1427, 1039-1295, 1039-1430, 1045-1283, 1047-1292, 1058-1312, 1060-1426, |
| | 1072-1372, 1083-1208, 1085-1327, 1096-1220, 1096-1423, 1106-1241, 1115-1427, 1116-1345, 1120-1428, |
| | 1121-1428, 1121-1592, 1122-1320, 1123-1380, 1123-1393, 1124-1430, 1127-1594, 1131-1427, 1132-1283, 1132-1430, |
| | 1133-1402, 1133-1406, 1135-1423, 1140-1412, 1143-1377, 1143-1413, 1149-1309, 1149-1581, 1152-1358, |
| | 1153-1596, 1159-1608, 1161-1430, 1164-1592, 1167-1592, 1174-1593, 1176-1439, 1177-1610, 1177-1614, 1180-1592, |
| | 1181-1430, 1188-1592, 1190-1592, 1192-1592, 1194-1589, 1194-1606, 1200-1430, 1201-1430, 1202-1284, |
| | 1203-1430, 1205-1428, 1207-1283, 1207-1472, 1210-1427, 1210-1592, 1212-1584, 1215-1427, 1219-1285, 1224-1282, |
| | 1227-1427, 1230-1620, 1232-1427, 1235-1427, 1236-1427, 1238-1592, 1239-1493, 1241-1430, 1243-1609, |
| | 1267-1546, 1280-1336, 1283-1592, 1284-1592, 1290-1589, 1294-1597, 1302-1592, 1325-1592, 1328-1427, 1337-1427, |
| | 1342-1586, 1342-1592, 1356-1505, 1361-1573, 1367-1540, 1367-1592, 1466-1582 |
| 94/7503568CB1/ | 1-199, 1-223, 1-262, 4-242, 4-293, 4-331, 8-270, 8-279, 9-251, 9-287, 9-298, 11-263, 11-270, 17-244, 18-282, 18-422, |
| 1444 | 19-240, 19-266, 19-276, 19-287, 19-474, 19-582, 20-294, 22-306, 23-297, 28-291, 28-315, 29-281, 29-308, 32-276, |
| | 32-322, 33-318, 34-286, 34-304, 34-317, 34-363, 34-486, 34-522, 34-559, 34-567, 34-568, 34-576, 34-598, 34-606, |
| | 34-1333, 35-244, 35-525, 37-287, 37-292, 37-626, 38-216, 38-250, 38-295, 38-297, 38-304, 39-312, 39-315, 39-316, |
| | 39-328, 39-429, 39-642, 39-673, 40-623, 41-539, 44-296, 45-271, 45-275, 45-286, 45-287, 45-288, 45-324, 46-342, |
| | 48-228, 48-510, 49-477, 51-369, 52-276, 52-297, 52-501, 52-680, 53-291, 53-308, 53-312, 53-337, 53-495, 56-254, |
| | 56-269, 56-286, 56-322, 56-626, 57-280, 57-306, 57-392, 58-265, 58-357, 60-284, 62-265, 62-283, 62-286, 62-335, |
| | 62-337, 62-339, 62-351, 62-395, 62-411, 63-690, 64-284, 64-293, 64-295, 64-302, 64-303, 64-325, 64-357, 65-249, |
| | 65-355, 66-301, 66-340, 67-313, 67-381, 70-513, 71-282, 71-300, 71-502, 71-683, 72-346, 72-524, 74-329, 74-646, |
| | 78-314, 78-375, 79-361, 80-295, 80-308, 80-349, 80-650, 81-362, 81-377, 82-342, 82-352, 84-265, |
| | 88-382, 90-294, 91-352, 91-676, 98-305, 98-310, 98-374, 98-375, 98-448, 98-651, 103-353, 104-363, 104-388, 104-647, |
| | 105-606, 106-392, 109-378, 112-414, 117-341, 118-345, 118-387, 121-629, 126-610, 128-365, 128-384, 130-292, |
| | 130-402, 131-402, 133-396, 133-410, 135-410, 136-428, 136-438, 136-459, 141-416, 150-371, 151-310, 151-452, |
| | 156-429, 156-444, 158-407, 158-414, 160-432, 162-402, 167-378, 171-451, 172-440, 176-382, 177-404, 186-452, |
| | 188-491, 191-465, 196-486, 197-415, 197-447, 197-460, 199-430, 199-483, 200-467, 203-503, 218-467, 219-451, |
| | 221-472, 222-446, 222-472, 222-478, 234-475, 234-502, 235-499, 238-510, 238-524, 239-515, 245-502, 245-503, |
| | 246-347, 246-405, 246-470, 247-516, 248-498, 250-502, 255-498, 259-477, 259-490, 259-494, 267-417, 267-529, |
| | 267-537, 267-541, 268-516, 268-523, 269-462, 269-514, 272-503, 272-533, 272-542, 273-658, 275-708, 279-503, |
| | 279-552, 282-522, 285-602, 288-549, 291-728, 296-547, 296-563, 301-796, 302-528, 303-536, 303-563, 303-598, |
| | 303-632, 304-500, 304-559, 306-539, 310-533, 310-561, 310-572, 317-572, 320-504, 321-580, 324-539, 324-541, |
| | 324-574, 324-577, 324-582, 324-627, 327-537, 327-541, 329-621, 336-639, 337-596, 339-594, 341-543, |
| | 342-629, 346-575, 348-612, 351-724, 437-671, 447-728, 577-1102, 577-1175, 581-1175, 627-727, 628-1131, 628-1174, |
| | 695-978, 729-822, 729-824, 729-897, 729-951, 729-965, 729-967, 729-988, 729-999, 729-1016, 729-1026, |
| | 729-1192, 729-1228, 729-1249, 729-1259, 729-1260, 729-1270, 729-1303, 729-1308, 729-1312, 730-1318, 733-966, |
| | 733-1029, 734-1007, 741-1084, 744-1009, 749-985, 753-952, 753-959, 753-963, 753-974, 753-1020, 756-1062, 756-1260, |
| | 757-997, 761-1318, 763-1003, 765-977, 765-1317, 768-1253, 774-1037, 775-986, 776-1231, 777-1274, 780-1065, |
| | 782-1036, 785-1056, 789-888, 790-1065, 796-1315, 797-1260, 797-1340, 798-1274, 801-972, 801-1052, 802-1113, |
| | 804-1062, 804-1344, 806-1039, 812-1287, 815-1022, 817-1091, 820-1076, 822-1275, 829-1159, 831-1092, |
| | 831-1095, 831-1273, 833-1080, 834-1311, 838-1107, 838-1127, 840-1075, 846-1279, 846-1304, 847-1215, 847-1342, |
| | 851-1115, 851-1127, 852-1113, 852-1275, 854-1173, 855-1117, 856-1279, 857-1183, 858-1080, 859-1010, |
| | 861-1276, 862-1274, 862-1275, 864-1375, 866-1232, 866-1275, 867-1161, 868-1091, 868-1124, 868-1127, 869-1279, |
| | 869-1444, 872-1344, 873-1275, 876-1113, 877-1084, 881-1129, 881-1275, |
| | 888-1159, 888-1275, 890-1085, 893-1077, 894-1104, 895-1076, 903-1209, 904-1141, 904-1148, 907-1125, 907-1165, |
| | 907-1295, 911-1131, 912-1185, 912-1358, 912-1363, 913-1066, 914-1155, 915-1168, 915-1275, 916-1189, |
| | 917-1154, 917-1338, 918-1218, 922-1327, 923-1109, 923-1149, 923-1194, 924-1184, 924-1336, 925-1052, 925-1275, |
| | 927-1275, 928-1138, 931-1164, 932-1211, 934-1275, 939-1175, 939-1274, 942-1275, 945-1188, 946-1161, |
| | 946-1234, 950-1256, 951-1273, 953-1263, 955-1226, 958-1442, 960-1222, 961-1213, 965-1265, 965-1278, 966-1226, |
| | 966-1227, 968-1214, 970-1253, 972-1185, 979-1111, 982-1277, 984-1198, 984-1264, 986-1248, 987-1155, |
| | 987-1263, 989-1276, 990-1232, 991-1234, 994-1220, 995-1444, 1007-1406, 1018-1187, 1058-1328, 1066-1236, |
| | 1066-1304, 1066-1332, 1244-1408 |
| 95/7504101CB1/ | 1-363, 1-391, 1-452, 1-469, 1-477, 1-481, 1-487, 1-500, 1-506, 1-662, 1-737, 1-2540, 1-2549, 11-379, 15-483, 16-479, |
| 2562 | 16-481, 78-398, 78-531, 78-619, 78-623, 78-630, 78-644, 78-660, 88-736, 101-838, 113-319, 205-914, 233-930, |
| | 265-400, 265-884, 333-900, 384-659, 424-1016, 460-725, 494-1147, 532-798, 568-1168, 574-884, 575-1237, |
| | 588-1157, 620-1308, 665-1119, 679-991, 679-1168, 687-1301, 694-1119, 706-976, 710-1114, 737-1323, 759-1119, |
| | 774-1132, 793-1126, 812-1427, 870-1138, 931-1675, 940-1116, 982-1671, 1039-1675, 1114-1322, 1196-1675, 1197-1695, |
| | 1220-1622, 1225-1622, 1258-1675, 1260-1712, 1263-1624, 1277-1847, 1278-1673, 1297-1908, 1357-1684, |
| | 1365-1730, 1377-1644, 1378-2182, 1393-1545, 1393-1609, 1393-2072, 1472-1964, 1483-1689, 1548-1895, 1561-2136, |
| | 1584-1977, 1639-1896, 1681-2007, 1719-1911, 1837-2068, 1954-2202, 1979-2201, 1979-2220, 2072-2539, |
| | 2081-2539, 2086-2539, 2148-2539, 2150-2528, 2157-2539, 2160-2539, 2214-2388, 2214-2562, 2223-2536, 2248-2387, |
| | 2253-2539, 2254-2539, 2257-2539, 2289-2535, 2289-2539, 2307-2539, 2314-2539, |
| | 2349-2539, 2349-2553, 2352-2530, 2393-2539 |
| 96/6946680CB1/ | 1-228, 108-444, 120-2189, 122-407, 122-998, 126-295, 131-714, 148-598, 355-2108, 1024-1410, 1025-1127, 1025-1626, |
| 2329 | 1061-1296, 1061-1670, 1064-1242, 1064-1847, 1065-1108, 1072-1118, 1086-1296, 1087-1200, 1151-1200, |
| | 1227-1877, 1232-1536, 1232-2015, 1233-1296, 1236-1588, 1253-1295, 1253-1588, 1255-1445, 1277-1610, 1277-1878, |
| | 1311-1961, 1320-1368, 1321-1588, 1397-1467, 1397-2006, 1401-1670, 1421-1626, 1423-1670, 1569-1612, |
| | 1649-1884, 1656-1717, 1656-1801, 1656-2008, 1673-2180, 1673-2200, 1673-2232, 1673-2329, 1675-1877, 1699-1877, |
| | 1759-1872, 1759-1880, 1759-1949, 1759-1961, 1908-1956, 1908-1961, 1912-1958 |
| 97/7001142CB1/ | 1-369, 1-451, 1-589, 1-606, 1-716, 1-717, 410-1140, 420-1140, 513-1140, 631-1136, 739-1806, 835-1091, 835-1099, |
| 1979 | 835-1384, 835-1401, 835-1455, 850-1007, 850-1013, 850-1050, 850-1300, 850-1343, 850-1612, 851-964, 851-967, |
| | 851-1009, 851-1028, 851-1040, 851-1061, 851-1093, 851-1149, 851-1185, 851-1199, 851-1277, 851-1418, 851-1512, |
| | 874-1168, 877-1199, 888-998, 889-1061, 901-1040, 901-1168, 901-1199, 911-966, 911-1204, 911-1269, 911-1535, |
| | 924-1168, 934-1175, 934-1181, 934-1192, 934-1215, 934-1426, 934-1485, 934-1511, 934-1696, 961-1277, |
| | 981-1168, 984-1277, 985-1161, 1009-1199, 1048-1418, 1065-1199, 1068-1418, 1069-1215, 1079-1208, 1079-1265, |
| | 1079-1300, 1079-1547, 1079-1551, 1079-1605, 1079-1703, 1103-1292, 1103-1386, 1103-1612, 1103-1679, 1103-1764, |
| | 1142-1512, 1143-1277, 1144-1512, 1153-1339, 1176-1418, 1179-1418, 1187-1427, 1187-1435, 1187-1470, |
| | 1187-1476, 1187-1700, 1187-1754, 1229-1535, 1240-1423, 1260-1512, 1263-1512, 1271-1511, 1271-1517, 1271-1528, |
| | 1271-1697, 1310-1612, 1320-1612, 1321-1507, 1347-1535, 1348-1900, |
| | 1355-1595, 1355-1603, 1355-1612, 1355-1625, 1355-1703, 1381-1914, 1405-1591, 1429-1612, 1462-1679, 1462-1722, |
| | 1462-1756, 1465-1696, 1479-1612, 1480-1696, 1489-1675, 1512-1979, 1515-1979, 1561-1733, 1563-1703, |
| | 1573-1697, 1596-1696, 1599-1696, 1644-1722, 1644-1754, 1647-1703, 1647-1764, 1656-1679, 1657-1703, 1720-1754 |
| 98/71158380CB1/ | 1-247, 1-299, 1-421, 1-476, 1-486, 1-521, 1-543, 6-660, 32-616, 129-521, 144-591, 228-766, 244-526, 333-960, 358-944 |
| 2795 | 371-878, 430-1074, 455-962, 516-725, 568-1057, 586-610, 586-827, 586-847, 586-1058, 586-1078, 586-1080, |
| | 624-681, 631-684, 643-698, 648-699, 687-1251, 699-847, 699-1080, 699-1084, 699-1111, 708-804, 708-857, 708-910, |
| | 708-911, 708-921, 708-925, 708-1030, 708-1085, 710-1393, 712-940, 733-1085, 735-1111, 746-838, 746-909, |
| | 746-924, 758-1319, 761-1085, 763-924, 765-1396, 791-1287, 799-1030, 800-841, 800-856, 800-858, 800-863, 800-873, |
| | 800-875, 800-893, 800-894, 800-922, 800-924, 800-943, 800-986, 800-1023, 800-1024, 806-1023, 808-841, |
| | 808-863, 808-875, 812-875, 816-927, 816-943, 816-1085, 816-1107, 819-924, 830-1023, 864-1085, 877-1023, 880-931, |
| | 880-974, 880-994, 880-996, 880-1005, 880-1023, 880-1026, 880-1107, 882-924, 883-1107, 883-1111, 884-957, |
| | 884-996, 884-1015, 884-1026, 884-1027, 884-1107, 888-1500, 890-1478, 905-1023, 911-1498, 914-1023, 919-1023, |
| | 934-1023, 935-1023, 952-1107, 961-1111, 964-999, 964-1023, 964-1111, 966-1023, |
| | 967-1098, 967-1107, 968-1090, 970-1107, 972-1023, 972-1107, 973-1107, 974-1107, 977-1023, 978-1023, 978-1107, |
| | 979-1034, 980-1034, 990-1111, 1000-1528, 1003-1107, 1004-1107, 1006-1030, 1006-1185, 1006-1266, 1006-1267, |
| | 1006-1490, 1008-1477, 1012-1111, 1016-1550, 1016-1568, 1033-1111, 1036-1107, 1041-1521, 1044-1101, |
| | 1044-1107, 1048-1110, 1048-1151, 1048-1188, 1048-1226, 1048-1269, 1048-1275, 1051-1107, 1056-1111, 1061-1111, |
| | 1062-1107, 1072-1680, 1073-1107, 1119-1528, 1130-1528, 1132-1267, 1136-1718, 1144-1675, 1145-1682, |
| | 1175-1776, 1210-1245, 1210-1282, 1210-1443, 1215-1848, 1216-1258, 1216-1267, 1216-1275, 1216-1419, 1216-1443 |
| | 1220-1260, 1220-1267, 1220-1273, 1220-1275, 1220-1285, 1220-1443, 1224-1267, 1224-1275, 1226-1267, |
| | 1229-1275, 1229-1443, 1230-1275, 1291-1848, 1296-1445, 1296-1513, 1296-1615, 1296-1670, 1300-1513, 1300-1670, |
| | 1304-1871, 1313-1780, 1338-1402, 1339-1402, 1340-2015, 1348-1443, 1348-1444, 1349-1670, 1353-1670, |
| | 1380-1425, 1380-1429, 1380-1437, 1380-1443, 1380-1524, 1380-1591, 1380-1615, 1384-1462, 1384-1527, 1384-1528, |
| | 1384-1562, 1384-1608, 1385-1425, 1388-1443, 1388-1606, 1388-1608, 1388-1611, 1392-1443, |
| | 1393-1443, 1397-1443, 1398-1443, 1400-1437, 1408-1497, 1408-1509, 1408-1524, 1408-1528, 1409-1944, 1411-1528, |
| | 1416-1528, 1418-1944, 1423-1528, 1424-1524, 1424-1528, 1439-1528, 1441-1747, 1463-1528, 1464-1513, |
| | 1464-1526, 1464-1578, 1464-1585, 1464-1608, 1464-1699, 1468-1503, 1468-1524, 1471-1699, 1471-1776, 1472-1611, |
| | 1476-1670, 1491-1611, 1512-1608, 1513-1666, 1517-1608, 1553-1608, 1556-1611, 1557-1603, 1557-1608, |
| | 1557-1611, 1557-1699, 1561-1610, 1562-1611, 1562-2193, 1569-1616, 1569-1620, 1569-1675, 1569-1774, 1569-1779, |
| | 1569-1780, 1569-1860, 1570-1611, 1571-1615, 1572-1613, 1583-1611, 1600-1779, 1642-1776, 1647-1851, |
| | 1656-1779, 1666-1942, 1667-1779, 1688-1778, 1688-2246, 1688-2350, 1688-2371, 1710-1746, 1710-1783, 1710-1938, |
| | 1710-1944, 1716-1779, 1720-1761, 1720-1773, 1720-1776, 1720-1780, 1720-1860, 1720-1948, 1721-1776, |
| | 1721-1784, 1721-1814, 1721-1863, 1721-1864, 1721-1871, 1721-1944, 1721-1947, 1725-1779, 1732-1776, 1732-1779, |
| | 1737-1779, 1737-1914, 1737-1947, 1737-1948, 1738-1779, 1748-1779, 1751-1863, 1751-1929, |
| | 1756-1860, 1766-1929, 1768-2001, 1768-2384, 1778-2360, 1789-1854, 1789-1860, 1798-1948, 1799-1948, 1800-2491, |
| | 1803-1860, 1804-1864, 1804-1944, 1805-1870, 1805-1926, 1805-1929, 1805-1947, 1807-2395, 1808-2399, |
| | 1814-2038, 1824-1947, 1825-1895, 1825-1914, 1825-1926, 1825-1948, 1826-1944, 1828-2508, 1833-2425, 1835-1944, |
| | 1835-1947, 1846-1948, 1847-1948, 1849-1944, 1856-1948, 1870-1948, 1880-1948, 1881-1944, 1884-1947, |
| | 1889-1944, 1889-1964, 1892-1939, 1892-1988, 1893-1986, 1893-1988, 1898-1947, 1898-1988, 1911-2528, 1923-1948, |
| | 1923-1987, 1927-1988, 1940-2217, 1940-2440, 1940-2585, 1962-2538, 1989-2188, 1989-2408, 1991-2046, |
| | 1992-2041, 1992-2043, 1992-2046, 1992-2047, 1992-2084, 1992-2087, 1992-2286, 1992-2309, 1993-2047, 1993-2074, |
| | 1996-2029, 1996-2063, 1997-2046, 2010-2087, 2026-2521, 2039-2087, 2045-2447, 2101-2126, 2125-2557, |
| | 2165-2353, 2167-2795, 2169-2538, 2335-2552 |
| 99/7503861CB1/ | 1-240, 1-562, 1-581, 3-4567, 4-642, 99-682, 99-774, 227-788, 248-848, 252-849, 299-939, 403-919, 405-876, 508-1156, |
| 4567 | 541-761, 580-1189, 603-1190, 665-1160, 717-1308, 726-1457, 818-1325, 837-1360, 848-1491, 876-1461, 879-1454, |
| | 886-1341, 891-1314, 894-1142, 943-1619, 948-1512, 948-1573, 952-1587, 1076-1476, 1094-1675, 1112-1625, |
| | 1114-1659, 1114-1661, 1132-1443, 1137-1739, 1150-1656, 1170-1698, 1192-1709, 1192-1721, 1196-1786, |
| | 1202-1752, 1207-1569, 1289-1764, 1313-1931, 1319-1933, 1322-1808, 1357-1901, 1369-1958, 1377-1553, 1484-1739, |
| | 1598-1796, 1640-1982, 1736-1833, 1913-2192, 1913-2194, 1913-2202, 1913-2205, 1913-2213, 1913-2215, |
| | 1913-2221, 1913-2224, 1913-2234, 2089-2576, 2216-2941, 2352-2588, 2352-2822, 2352-2823, 2352-2842, 2352-2854, |
| | 2352-2883, 2352-2888, 2352-2889, 2352-2898, 2352-2900, 2352-2994, 2352-3008, 2367-3011, 2379-2941, |
| | 2436-2995, 2436-3035, 2450-3037, 2457-2964, 2471-3065, 2474-2709, 2499-3182, 2519-2744, 2542-3170, 2585-3078, |
| | 2591-3176, 2595-3170, 2598-3149, 2601-3066, 2601-3247, 2614-3189, 2652-2800, 2661-3224, |
| | 2662-3199, 2664-2967, 2664-3357, 2688-2953, 2691-3023, 2710-3332, 2772-2907, 2817-3447, 2848-3436, 2866-3398, |
| | 2909-3186, 2909-3229, 2909-3329, 2909-3413, 2910-3244, 2920-3508, 2944-3568, 2963-3486, 2963-3687, |
| | 2971-3266, 2980-3345, 2993-3230, 3021-3654, 3033-3288, 3045-3295, 3081-3747, 3104-3497, 3114-3600, 3122-3362, |
| | 3131-3753, 3135-3391, 3150-3461, 3167-3446, 3184-3683, 3194-3416, 3194-3426, 3215-3424, 3231-3746, |
| | 3259-3759, 3266-3758, 3277-3745, 3283-4066, 3304-3759, 3399-3654, 3414-3641, 3418-3948, 3441-3757, 3465-3742, |
| | 3466-4074, 3478-3900, 3523-3793, 3532-3792, 3557-3844, 3573-3836, 3580-4066, 3586-3896, 3619-3756, |
| | 3625-3926, 3651-3971, 3658-3872, 3680-3843, 3685-4379, 3690-3758, 3690-3930, 3690-4262, 3725-4014, 3757-4039, |
| | 3758-4018, 3758-4034, 3760-4401, 3760-4422, 3761-3967, 3761-4006, 3761-4313, 3762-4028, 3780-4042, |
| | 3780-4045, 3794-4059, 3804-4513, 3804-4550, 3834-4099, 3835-4282, 3835-4367, 3847-4094, 3847-4398, |
| | 3875-4441, 3879-4148, 3884-4379, 3892-4196, 3896-4167, 3898-4519, 3900-4334, 3923-4560, 3941-4200, 3955-4234, |
| | 3965-4520, 3965-4538, 3968-4104, 3980-4519, 3981-4253, 3984-4446, 4007-4490, 4007-4559, 4007-4564, |
| | 4015-4259, 4043-4485, 4043-4557, 4043-4567, 4069-4567, 4072-4300, 4074-4567, 4077-4567, 4085-4558, 4098-4326, |
| | 4102-4557, 4104-4559, 4109-4550, 4137-4560, 4142-4559, 4143-4559, 4145-4559, 4146-4557, 4149-4559, |
| | 4156-4376, 4159-4560, 4160-4558, 4160-4559, 4168-4460, 4171-4559, 4173-4563, 4174-4563, 4180-4446, 4180-4558, |
| | 4180-4559, 4207-4556, 4207-4560, 4221-4483, 4224-4325, 4232-4424, 4232-4545, 4232-4567, 4245-4559, |
| | 4258-4561, 4268-4560, 4270-4567, 4293-4559, 4297-4479, 4297-4526, 4307-4567, 4317-4559, 4371-4553, 4422-4559, |
| | 4434-4535 |
| 100/7758395CB1/ | 1-630, 1-4506, 230-809, 269-964, 302-900, 302-969, 528-1085, 729-1345, 741-1107, 759-1095, 792-1040, 814-1396, |
| 4507 | 856-1357, 946-1354, 957-1415, 991-1664, 1026-1539, 1312-2121, 1318-2056, 1318-2064, 1318-2107, 1318-2136, |
| | 1319-2024, 1319-2120, 1329-1832, 1329-1910, 1397-1795, 1430-2066, 1477-2038, 1555-2135, 1557-2222, |
| | 1659-2305, 1712-1979, 1712-2005, 1712-2110, 1712-2164, 1712-2192, 1712-2242, 1714-2063, 1730-2180, 1797-2266, |
| | 1844-2659, 1902-2658, 1946-2658, 1948-2592, 1948-2594, 1948-2658, 1953-2583, 1957-2594, 1971-2658, |
| | 2008-2658, 2011-2658, 2012-2692, 2072-2687, 2072-2771, 2077-2475, 2183-2906, 2514-2775, 2514-3132, 2533-2817, |
| | 2567-2898, 2567-3034, 2567-3198, 2582-2994, 2694-3020, 2896-3467, 2938-3788, 2944-3162, 2944-3164, |
| | 2944-3302, 2944-3439, 2944-3558, 2944-3681, 2970-3420, 2991-3285, 3028-3673, 3035-3709, 3035-3720, 3098-3687, |
| | 3112-3688, 3139-3821, 3150-3768, 3180-3694, 3181-3694, 3182-3787, 3216-3793, 3264-3829, 3278-3831, |
| | 3302-3587, 3309-3830, 3327-3512, 3345-3831, 3387-3831, 3390-3831, 3415-3818, 3424-3685, 3424-3695, |
| | 3435-3831, 3438-3591, 3459-3818, 3500-3785, 3500-3844, 3500-3868, 3568-3831, 3575-4243, 3614-3751, 3664-3788, |
| | 3671-3840, 3713-3815, 3713-3909, 3817-4507 |
| 101/71039312CB1/ | 1-320, 46-319, 46-352, 46-471, 137-438, 161-503, 161-4845, 265-837, 317-569, 657-903, 657-1274, 721-933, 874-1552, |
| 4862 | 879-1552, 937-1211, 1065-1717, 1192-1621, 1192-1623, 1223-1616, 1228-1581, 1284-1418, 1295-1325, 1595-1845, |
| | 1595-1864, 1595-1949, 1595-2135, 1635-1948, 1658-1914, 1759-2361, 1759-2371, 1929-2645, 1946-2444, |
| | 1959-2349, 2056-2450, 2058-2556, 2058-2560, 2098-2309, 2098-2614, 2149-2693, 2150-2788, 2150-2823, 2152-2444, |
| | 2156-2412, 2158-2836, 2317-2568, 2317-2739, 2332-2517, 2360-2583, 2584-2922, 2585-3186, 2753-3039, |
| | 2770-3428, 2856-3181, 2869-3244, 2931-3544, 2943-3532, 2962-3619, 3006-3634, 3007-3212, 3007-3459, 3130-3675, |
| | 3150-3683, 3156-3822, 3168-3753, 3178-3822, 3189-3781, 3320-3851, 3326-3848, 3351-3812, 3367-3851, |
| | 3395-3641, 3485-3763, 3570-4131, 3618-3837, 3962-4419, 4166-4375, 4222-4665, 4426-4859, 4426-4862, 4842-4862 |
| 102/7291318CB1/ | 1-562, 192-897, 516-688, 719-897, 719-1298, 776-893, 784-1250, 784-1341, 983-2537, 1024-1299, 1449-2075, |
| 3011 | 1472-2060, 1514-1960, 1635-1810, 1734-2028, 2050-2532, 2308-2572, 2375-2858, 2402-3011, 2418-2930 |
| 103/2638619CB1/ | 1-147, 1-1662, 56-296, 56-339, 56-425, 56-831, 58-327, 58-504, 60-879, 61-191, 80-747, 122-666, 122-859, 147-914, |
| 3092 | 149-647, 1112-1459, 1289-1780, 1431-1699, 1435-1648, 1435-1649, 1446-1922, 1446-1929, 1447-2174, 1530-2045, |
| | 1531-1731, 1593-2263, 1595-2226, 1663-1880, 1693-1888, 1693-2088, 1740-2313, 1744-1949, 1763-1967, |
| | 1795-2076, 1811-2039, 1832-2479, 1835-2336, 1891-2525, 1946-2208, 2063-2671, 2065-2313, 2065-2420, 2065-2550, |
| | 2086-2801, 2103-2714, 2127-2749, 2136-2224, 2136-2232, 2145-2744, 2166-2491, 2192-2414, 2194-2772, |
| | 2211-2425, 2211-2689, 2222-2763, 2233-2483, 2233-2616, 2233-2793, 2244-2816, 2246-2493, 2246-2721, 2247-2440, |
| | 2249-2569, 2253-2630, 2254-2501, 2254-2810, 2254-2811, 2257-2590, 2267-2529, 2282-2793, 2292-2765, |
| | 2294-2809, 2300-2504, 2310-2793, 2329-2804, 2335-2795, 2341-2468, 2345-2629, 2352-2805, 2365-2456, 2365-2795, |
| | 2367-2425, 2367-2740, 2367-2794, 2372-2810, 2375-2795, 2377-2667, 2377-2784, 2383-2801, 2385-2672, |
| | 2400-2794, 2400-2796, 2405-2800, 2407-2797, 2408-3092, 2411-2801, 2413-2797, 2415-2794, 2416-2803, |
| | 2438-2801, 2444-2732, 2450-2817, 2479-2795, 2491-2815, 2502-2803, 2503-2680, 2505-2793, 2508-2777, 2532-2758, |
| | 2537-2820, 2542-2797, 2542-2801, 2548-2801, 2555-2800, 2563-2785, 2563-2797, 2564-2797, 2570-2801, |
| | 2585-2803, 2600-2815, 2631-2801, 2669-2820, 2686-2803, 2718-2795 |
| 104/2810014CB1/ | 1-296, 1-628, 17-303, 137-391, 179-841, 229-779, 275-836, 275-854, 362-879, 374-627, 425-688, 581-713 |
| 879 | |
| 105/3457155CB1/ | 1-3711, 940-1558, 940-1680, 942-1694, 989-1660, 1095-1325, 1105-1589, 1112-1356, 1328-1694, 1474-2105, 1491-2128, |
| 4574 | 1613-2117, 1624-2117, 1625-2178, 1774-2089, 1775-2064, 1929-2524, 1979-2615, 2004-2554, 2095-2293, |
| | 2095-2377, 2095-2504, 2095-2590, 2095-2684, 2095-2727, 2161-2769, 2334-2580, 2334-2815, 2334-2848, 2334-2861, |
| | 2334-2910, 2334-2929, 2334-2930, 2337-2876, 2352-2459, 2424-2691, 2424-2789, 2424-2920, 2436-2743, |
| | 2438-2898, 2461-2979, 2464-3100, 2584-3274, 2586-2825, 2672-3451, 2696-3363, 2706-3363, 2728-3101, 2734-3278, |
| | 2741-3382, 2806-3390, 2821-3216, 2893-3335, 2909-3495, 2915-3443, 2923-3533, 2924-3206, 2924-3667, |
| | 2926-3540, 2947-3518, 2953-3196, 2975-3305, 2976-3494, 2979-3423, 2985-3718, 2991-3528, 2991-3552, 3005-3631, |
| | 3007-3584, 3014-3240, 3023-3607, 3024-3164, 3040-3101, 3051-3291, 3053-3590, 3066-3515, 3082-3750, |
| | 3095-3692, 3098-3515, 3119-3399, 3119-3612, 3123-3727, 3130-3518, 3167-3605, 3193-3784, 3193-3829, 3200-3911, |
| | 3215-3468, 3215-3761, 3227-3713, 3286-3881, 3290-3816, 3296-3753, 3296-3945, 3298-3558, |
| | 3304-3863, 3307-3559, 3315-3580, 3321-3938, 3350-3601, 3362-3849, 3383-3939, 3403-3742, 3404-3999, 3405-4037, |
| | 3418-3958, 3420-3882, 3448-4135, 3452-4077, 3458-4094, 3461-4044, 3476-4011, 3477-3999, 3485-4130, |
| | 3493-3909, 3502-4057, 3506-3923, 3506-4025, 3511-3987, 3514-4164, 3527-4114, 3531-3975, 3554-3847, 3565-4176, |
| | 3565-4240, 3572-4037, 3574-4196, 3581-4221, 3606-4211, 3611-4053, 3631-3911, 3642-4169, 3642-4492, |
| | 3645-4055, 3651-4185, 3663-4073, 3694-4114, 3695-4407, 3697-4131, 3718-3994, 3723-4011, 3735-4012, 3735-4220, |
| | 3783-4380, 3823-4490, 3836-4343, 3907-4526, 3910-4486, 3921-4524, 3922-4535, 3946-4372, 3946-4494, |
| | 3961-4379, 3965-4556, 3981-4479, 3982-4269, 4018-4530, 4023-4430, 4051-4515, 4055-4529, 4064-4453, 4091-4533, |
| | 4099-4574, 4108-4532, 4113-4532, 4118-4531, 4122-4531, 4124-4532, 4138-4531, 4139-4407, 4161-4532, |
| | 4163-4530, 4165-4531, 4165-4532, 4183-4399, 4186-4458, 4186-4530, 4190-4532, |
| | 4191-4466, 4191-4500, 4191-4532, 4195-4533, 4201-4531, 4211-4532, 4214-4532, 4225-4531, 4241-4470, 4252-4532, |
| | 4280-4530, 4302-4532, 4323-4530, 4330-4532, 4400-4532, 4403-4569 |
| 106/7435171CB1/ | 1-1074, 224-550, 431-1007, 432-976, 911-1483 |
| 1483 | |
| 107/7499936CB1/ | 1-994, 55-761, 55-762, 103-385, 104-761, 105-579, 123-435, 127-762, 130-762, 214-730, 214-733, 238-612, 287-845, |
| 994 | 296-610, 313-742, 318-891, 320-848, 325-611, 328-745, 332-823, 356-748, 373-989, 385-745, 404-659, 408-735, |
| | 409-742, 423-989, 428-703, 428-704, 429-713, 477-952, 488-774, 509-984, 511-745, 517-746, 522-783, 522-984, |
| | 527-728, 527-749, 531-975, 548-983, 548-984, 625-743, 684-936, 684-970, 685-982, 694-953, 700-864, 739-984, |
| | 809-984 |
| 108/7504125CB1/ | 1-208, 1-1158, 59-729, 59-735, 59-736, 59-737, 73-736, 74-291, 89-736, 119-292, 187-736, 290-722, 290-937, 291-554, |
| 1179 | 295-1011, 306-543, 310-715, 355-579, 366-565, 398-627, 398-636, 412-1013, 442-701, 462-1099, 476-635, |
| | 485-931, 488-695, 490-800, 491-1115, 547-1114, 548-1179, 554-831, 564-1155, 588-736, 592-1059, 662-1158, 669-1154, |
| | 669-1179, 688-952, 706-928, 706-1174, 710-959, 747-1158, 765-985, 775-1017, 790-1100, 813-1148, 852-1158, |
| | 859-986, 859-1100, 882-1138, 911-1158, 935-1146, 935-1158, 936-1158, 1078-1179 |
| 109/7505742CB1/ | 1-2556, 9-137, 55-361, 56-317, 182-361, 188-352, 243-357, 578-1023, 578-2570, 672-929, 707-827, 709-1167, 710-1325, |
| 2622 | 712-1325, 714-1325, 723-914, 723-946, 723-1301, 740-1325, 743-1320, 765-1325, 772-940, 866-1325, 955-1407, |
| | 986-1323, 989-1487, 991-1436, 996-1452, 1041-1325, 1041-1498, 1062-1681, 1103-1700, 1132-1285, 1142-1398, |
| | 1146-1734, 1167-1469, 1167-1732, 1234-1425, 1253-1803, 1264-1684, 1269-1648, 1283-1839, 1297-1756, |
| | 1298-1869, 1315-1901, 1353-1894, 1359-2074, 1371-1850, 1382-1836, 1382-1924, 1384-1882, 1405-1700, 1430-1890, |
| | 1468-1729, 1470-1925, 1471-1777, 1496-1770, 1498-1661, 1504-1997, 1505-1756, 1522-2073, 1524-1727, |
| | 1545-1802, 1647-2209, 1656-2170, 1683-2017, 1683-2166, 1692-1897, 1712-1928, 1715-2277, 1786-2017, 1806-2036, |
| | 1806-2302, 1808-2027, 1830-2467, 1832-2118, 1890-2137, 1903-2153, 1942-2118, 1947-2205, 1949-2522, |
| | 1980-2170, 1995-2227, 2000-2268, 2028-2545, 2042-2290, 2044-2553, 2056-2199, 2075-2373, 2098-2565, 2102-2565, |
| | 2106-2321, 2114-2559, 2144-2622, 2148-2554, 2155-2622, 2195-2363, 2214-2523, 2261-2567, 2273-2537, |
| | 2299-2569, 2345-2566 |
| 110/7505757CB1/ | 1-3825, 1054-1672, 1054-1794, 1056-1808, 1103-1774, 1209-1439, 1219-1703, 1226-1470, 1442-1808, 1588-2219, |
| 4688 | 1605-2242, 1727-2231, 1738-2231, 1739-2292, 1888-2203, 1889-2178, 2043-2638, 2093-2729, 2118-2668, 2209-2407, |
| | 2209-2491, 2209-2618, 2209-2704, 2209-2798, 2209-2841, 2275-2883, 2448-2694, 2448-2929, 2448-2962, |
| | 2448-2975, 2448-3024, 2448-3043, 2448-3044, 2451-2990, 2466-2573, 2538-2805, 2538-2903, 2538-3034, 2550-2857, |
| | 2552-3012, 2575-3093, 2578-3214, 2698-3388, 2700-2939, 2786-3565, 2810-3477, 2820-3477, 2842-3215, |
| | 2848-3392, 2855-3496, 2920-3504, 2935-3330, 3007-3449, 3023-3609, 3029-3557, 3037-3647, 3038-3320, 3038-3781, |
| | 3040-3654, 3061-3632, 3067-3310, 3089-3419, 3090-3608, 3093-3537, 3099-3832, 3105-3642, 3105-3666, |
| | 3119-3745, 3121-3698, 3128-3354, 3137-3721, 3138-3278, 3154-3215, 3165-3405, 3167-3704, 3180-3629, 3196-3864, |
| | 3209-3806, 3212-3629, 3233-3513, 3233-3726, 3237-3841, 3244-3632, 3281-3719, 3307-3898, 3307-3943, |
| | 3314-4025, 3329-3582, 3329-3875, 3341-3827, 3400-3995, 3404-3930, 3410-3867, 3410-4059, 3412-3672, |
| | 3418-3977, 3421-3673, 3429-3694, 3435-4052, 3464-3715, 3476-3963, 3497-4053, 3517-3856, 3518-4113, 3519-4151, |
| | 3532-4072, 3534-3996, 3562-4249, 3566-4191, 3572-4208, 3575-4158, 3590-4125, 3591-4113, 3599-4244, |
| | 3607-4023, 3616-4171, 3620-4037, 3620-4139, 3625-4101, 3628-4278, 3641-4228, 3645-4089, 3668-3961, 3679-4290, |
| | 3679-4354, 3686-4151, 3688-4310, 3695-4335, 3720-4325, 3725-4167, 3745-4025, 3756-4283, 3756-4606, |
| | 3759-4169, 3765-4299, 3777-4187, 3808-4228, 3809-4521, 3811-4245, 3832-4108, 3837-4125, 3849-4126, 3849-4334, |
| | 3897-4494, 3937-4604, 3950-4457, 4015-4440, 4021-4640, 4024-4600, 4035-4638, 4036-4649, 4060-4608, |
| | 4075-4493, 4079-4670, 4095-4593, 4132-4644, 4137-4544, 4165-4629, 4169-4643, 4178-4567, 4205-4647, 4213-4688, |
| | 4222-4646, 4227-4646, 4232-4645, 4236-4645, 4238-4646, 4252-4645, 4253-4521, 4275-4646, |
| | 4277-4644, 4279-4645, 4279-4646, 4297-4513, 4300-4572, 4300-4644, 4304-4646, 4305-4580, 4305-4614, 4305-4646, |
| | 4309-4647, 4315-4645, 4325-4646, 4328-4646, 4339-4645, 4355-4584, 4366-4646, 4394-4644, 4416-4646, |
| | 4437-4644, 4444-4646, 4514-4646, 4517-4683 |
| 111/7504126CB1/ | 1-96, 1-142, 1-276, 1-368, 1-382, 2-92, 2-97, 2-101, 2-114, 2-140, 2-143, 2-149, 2-250, 2-314, 3-117, 3-135, 3-269, |
| 490 | 4-284, 5-285, 6-295, 7-286, 8-245, 8-308, 10-276, 13-149, 13-275, 18-135, 18-222, 28-160, 30-129, 126-378, 163-397, |
| | 164-396, 168-353, 180-401, 180-402, 197-379, 197-447, 206-318, 206-382, 226-400, 234-441, 236-447, 239-430, |
| | 242-414, 242-490, 254-382, 257-352, 269-340 |
| 112/7504099CB1/ | 1-259, 1-1199, 34-249, 59-259, 65-217, 70-259, 72-179, 78-185, 79-239, 87-259, 89-259, 89-525, 89-548, 89-557, |
| 1408 | 257-488, 271-704, 305-578, 305-840, 334-804, 336-626, 341-880, 353-584, 353-585, 370-733, 371-660, 411-1082, |
| | 424-685, 424-709, 426-684, 434-1000, 445-721, 446-700, 450-700, 457-737, 467-699, 477-1022, 483-757, 500-771, |
| | 505-1183, 508-1133, 512-757, 512-1057, 514-889, 526-754, 526-757, 526-760, 526-766, 526-769, 526-770, 526-772, |
| | 526-794, 530-716, 541-773, 541-838, 541-1215, 558-858, 563-768, 564-786, 582-723, 587-825, 587-837, 599-840, |
| | 612-852, 614-866, 626-780, 632-915, 644-904, 644-964, 669-907, 671-1135, 685-944, 706-897, 706-970, 706-1184, |
| | 706-1190, 712-998, 713-1011, 714-1006, 721-987, 748-1193, 754-1037, 755-1032, 760-1056, 764-1199, 765-950, |
| | 765-1021, 780-1193, 790-1162, 794-1079, 826-1165, 826-1267, 837-1077, 837-1081, 845-1408, 857-1052, 862-1108, |
| | 865-1128, 865-1148, 897-1136, 897-1144, 897-1170, 937-1179, 948-1188, 962-1258 |
| 113/7505733CB1/ | 1-600, 321-590, 331-455, 338-600, 347-587, 349-594, 358-577, 365-600, 375-766, 386-595, 395-600, 402-602, 444-600, |
| 1363 | 478-590, 478-600, 478-1363, 479-594, 479-602, 656-781, 656-1156, 1011-1154, 1144-1346 |
| 114/7959829CB1/ | 1-103, 1-593, 12-173, 238-738, 256-515, 293-484, 327-605, 439-1071 |
| 1071 | |
| 115/7502168CB1/ | 1-256, 51-362, 81-256, 180-256, 190-235, 190-443, 273-939, 280-1049, 763-1164, 763-1420, 931-1561, 1088-1585, |
| 2140 | 1316-1676, 1617-1981, 1617-2017, 1617-2018, 1617-2043, 1617-2048, 1617-2066, 1617-2075, 1617-2104, 1619-2013, |
| | 1620-2076, 1621-1809, 1623-1977, 1624-2140, 1634-2140 |
| 116/7503888CB1/ | 1-245, 1-685, 5-4956, 20-686, 27-267, 36-277, 44-306, 53-356, 55-486, 67-334, 68-729, 74-350, 75-333, 80-380, |
| 4980 | 110-182, 159-933, 165-816, 171-853, 173-460, 173-742, 173-803, 173-1010, 174-441, 174-850, 280-880, 411-934, |
| | 411-943, 456-671, 460-623, 501-792, 514-867, 524-730, 582-876, 590-1045, 602-854, 602-880, 606-1101, 624-843, |
| | 653-756, 684-1177, 756-999, 927-1153, 983-1481, 991-1405, 1014-1286, 1143-1631, 1143-1650, 1167-1443, 1169-1919, |
| | 1190-1450, 1193-1440, 1195-1417, 1196-1475, 1203-1883, 1259-1713, 1261-1979, 1269-1731, 1276-1579, |
| | 1282-1848, 1283-1538, 1286-1563, 1309-1912, 1317-1605, 1318-1974, 1331-1618, 1345-1627, 1348-1873, 1365-1633, |
| | 1366-1652, 1367-1880, 1373-1608, 1395-1670, 1397-1592, 1398-1888, 1398-1929, 1405-1969, 1405-1975, |
| | 1422-1775, 1423-1723, 1428-1948, 1434-1964, 1436-1676, 1436-1958, 1442-1748, 1451-1676, 1464-1591, 1464-2210, |
| | 1466-1731, 1485-1975, 1486-1726, 1490-1782, 1495-1749, 1496-1892, 1501-1770, 1501-1799, 1508-1952, |
| | 1508-1956, 1509-1961, 1520-1964, 1533-1975, 1535-1964, 1536-1812, 1536-1952, 1536-2137, 1540-2167, |
| | 1541-1803, 1550-1955, 1552-1923, 1554-1955, 1559-1955, 1561-1955, 1562-1955, 1566-1955, 1568-2009, 1586-1955, |
| | 1593-1865, 1604-2206, 1605-1975, 1606-1906, 1619-1862, 1626-2220, 1629-1956, 1631-1960, 1643-2162, |
| | 1652-1920, 1658-1955, 1659-1958, 1666-1964, 1686-1964, 1703-1951, 1711-2286, 1712-2564, 1751-1964, 1755-1905, |
| | 1768-1950, 1772-1975, 1774-2570, 1785-1960, 1785-1974, 1808-1954, 1808-1958, 1817-2100, 1822-1955, |
| | 1847-1955, 1848-1955, 1856-1975, 1898-2226, 1973-2287, 1978-2579, 1983-2097, 1985-2209, 2001-2209, 2021-2458, |
| | 2022-2164, 2023-2178, 2139-2687, 2204-3005, 2213-2739, 2214-2500, 2217-2520, 2246-2435, 2247-2853, |
| | 2273-2773, 2283-2414, 2312-2579, 2342-2659, 2353-2924, 2363-2988, 2364-2618, 2364-2885, 2368-2846, 2368-2881, |
| | 2381-2625, 2382-2904, 2389-2967, 2430-3262, 2448-2688, 2458-3063, 2459-3215, 2460-2955, 2465-3044, |
| | 2478-2716, 2492-3017, 2500-3058, 2500-3176, 2516-3168, 2519-2640, 2535-3194, 2537-2761, 2540-3291, |
| | 2545-3106, 2552-2867, 2563-2863, 2588-2864, 2630-2882, 2630-2888, 2678-2939, 2681-2944, 2714-3355, 2723-3186, |
| | 2724-3216, 2728-3198, 2744-3301, 2748-3264, 2777-3460, 2793-3240, 2796-3326, 2800-3095, 2812-3087, |
| | 2813-3035, 2819-3367, 2823-3284, 2830-3043, 2839-3346, 2839-3539, 2842-3456, 2849-3456, 2856-3060, 2859-3408, |
| | 2860-3169, 2860-3273, 2861-3601, 2875-3147, 2878-3078, 2884-3487, 2891-3083, 2893-3507, 2901-3461, |
| | 2906-3182, 2909-3203, 2910-3378, 2912-3155, 2914-3221, 2930-3167, 2930-3445, 2932-3321, 2936-3542, 2964-3223, |
| | 2967-3510, 2969-3410, 2969-3519, 2974-3413, 2975-3545, 3003-3223, 3020-3297, 3025-3255, 3025-3514, |
| | 3026-3492, 3028-3162, 3050-3617, 3051-3168, 3051-3491, 3051-3514, 3070-3345, 3082-3665, 3093-3815, 3094-3658, |
| | 3095-3371, 3101-3286, 3101-3652, 3103-3334, 3103-3697, 3104-3815, 3112-3625, 3117-3779, 3118-3412, |
| | 3126-3659, 3130-3638, 3143-3383, 3143-3422, 3161-3440, 3168-3462, 3172-3413, 3182-3467, 3185-3711, 3187-3441, |
| | 3197-3453, 3201-3484, 3207-3421, 3207-3446, 3216-3667, 3217-3303, 3224-3303, 3227-3475, |
| | 3231-3740, 3251-3485, 3255-3530, 3261-3814, 3267-3698, 3268-3653, 3279-3450, 3279-3625, 3284-3803, 3306-3539, |
| | 3314-3581, 3327-3810, 3329-3561, 3331-3697, 3361-3505, 3362-3815, 3368-3602, 3374-3627, 3389-3596, |
| | 3408-3785, 3418-3670, 3433-3668, 3439-3765, 3456-3663, 3461-3708, 3461-3717, 3461-3784, 3467-3631, 3475-3727, |
| | 3476-3637, 3483-3815, 3503-3657, 3503-3782, 3517-3788, 3540-3815, 3552-3761, 3554-3814, 3567-3815, |
| | 3569-3815, 3571-3810, 3571-3815, 3573-3781, 3590-3815, 3618-3780, 3618-3786, 3662-3807, 3813-4078, 3813-4084, |
| | 3813-4088, 3813-4090, 3813-4121, 3819-4076, 3845-4061, 3849-4059, 3849-4084, 3850-4092, 3867-4063, |
| | 3872-4592, 3875-4084, 3875-4313, 3942-4243, 3948-4784, 4042-4390, 4085-4378, 4085-4397, 4085-4402, 4085-4404, |
| | 4085-4418, 4085-4428, 4085-4439, 4085-4447, 4085-4450, 4085-4454, 4085-4456, 4085-4462, 4085-4466, |
| | 4085-4470, 4085-4481, 4085-4486, 4085-4567, 4085-4581, 4086-4468, 4087-4208, 4087-4470, 4088-4634, 4088-4707, |
| | 4089-4495, 4089-4567, 4091-4644, |
| | 4093-4385, 4093-4439, 4093-4470, 4093-4526, 4093-4527, 4093-4537, 4093-4550, 4093-4555, 4093-4566, 4093-4572, |
| | 4093-4587, 4093-4588, 4093-4591, 4093-4676, 4093-4770, 4095-4595, 4099-4478, 4100-4339, 4100-4592, |
| | 4103-4446, 4103-4455, 4103-4467, 4103-4469, 4103-4470, 4109-4566, 4111-4461, 4116-4204, 4118-4417, 4119-4466, |
| | 4120-4306, 4127-4590, 4130-4504, 4133-4392, 4141-4393, 4149-4576, 4151-4382, 4151-4470, 4151-4844, |
| | 4161-4567, 4163-4408, 4176-4434, 4178-4466, 4178-4490, 4180-4392, 4182-4414, 4190-4926, 4199-4455, 4201-4514, |
| | 4202-4511, 4225-4425, 4225-4448, 4233-4702, 4235-4702, 4238-4468, 4251-4694, 4266-4526, 4290-4572, |
| | 4319-4611, 4323-4557, 4325-4928, 4328-4607, 4357-4631, 4385-4819, 4392-4940, 4411-4694, 4415-4705, 4416-4747, |
| | 4419-4644, 4419-4823, 4420-4940, 4425-4980, 4431-4702, 4439-4581, 4439-4702, 4449-4925, 4452-4644, |
| | 4452-4792, 4462-4940, 4463-4729, 4463-4828, 4464-4747, 4465-4932, 4467-4960, 4469-4728, 4472-4708, 4483-4765, |
| | 4484-4768, 4484-4952, 4488-4731, 4496-4703, 4502-4774, 4510-4929, 4511-4718, 4518-4774, |
| | 4522-4732, 4523-4940, 4525-4780, 4533-4940, 4533-4970, 4538-4785, 4549-4940, 4557-4973, 4558-4961, 4567-4846, |
| | 4574-4883, 4579-4819, 4604-4927, 4625-4952, 4646-4917, 4654-4852, 4656-4895, 4656-4952, 4660-4931, |
| | 4661-4941, 4663-4940, 4667-4952, 4668-4889, 4668-4891, 4670-4921, 4670-4923, 4672-4891, 4672-4974, 4684-4945, |
| | 4700-4930, 4702-4952, 4704-4970, 4729-4933, 4783-4915, 4792-4980 |

TABLE 5

| | | |
|---|---|---|
| Polynucleotide SEQ | | Representative |
| ID NO: | Incyte Project ID: | Library |

| | | |
|---|---|---|
| 59 | 7503848CB1 | 293TF5T01 |
| 60 | 2608080CB1 | BRAIFEE05 |
| 61 | 7503402CB1 | GBLATUT01 |
| 62 | 7503517CB1 | PANCNOT05 |
| 63 | 7500014CB1 | NERDTDN03 |
| 64 | 7501365CB1 | HEAONOE01 |
| 65 | 7503540CB1 | SCORNON02 |
| 66 | 7504326CB1 | BRAUNOR01 |
| 67 | 7504388CB1 | BRAITUT12 |
| 68 | 2828380CB1 | PANCNOE02 |
| 69 | 6456919CB1 | LUNLTUT11 |
| 70 | 7502244CB1 | CONTTUT01 |
| 71 | 7498718CB1 | CERVNOT01 |
| 72 | 6259308CB1 | KIDEUNE02 |
| 73 | 7504104CB1 | UTRSDIC01 |
| 74 | 7504121CB1 | KIDEUNE02 |
| 75 | 5635695CB1 | UTRSTMR01 |
| 76 | 7503983CB1 | FIBRUNT02 |
| 77 | 7503476CB1 | PANCTUT02 |
| 78 | 7504023CB1 | COLNPOT01 |
| 79 | 7504128CB1 | PANCNOT04 |
| 80 | 4529338CB1 | HEARNON03 |
| 81 | 7503460CB1 | EPIPNOT01 |
| 82 | 5466630CB1 | COLENOR03 |
| 83 | 7503474CB1 | PANCNOT05 |
| 84 | 7503498CB1 | ENDCNOT03 |
| 85 | 7504119CB1 | MUSCNOT10 |
| 86 | 71532805CB1 | BRAIFEN03 |
| 87 | 5502992CB1 | THYMNOE02 |
| 88 | 7503828CB1 | BRACNOK02 |
| 89 | 2647325CB1 | PROSTME06 |
| 90 | 7495416CB1 | UTRCDIE01 |
| 91 | 8096177CB1 | TESTNON04 |
| 92 | 666763CB1 | OVARDIJ01 |
| 93 | 7504091CB1 | HNT2RAT01 |
| 94 | 7503568CB1 | UTRSNOT02 |
| 95 | 7504101CB1 | THYRDIE01 |
| 96 | 6946680CB1 | BRAENOT02 |
| 97 | 7001142CB1 | MMLR3DT01 |
| 98 | 71158380CB1 | MCLDTXN05 |
| 99 | 7503861CB1 | FIBRTXS07 |
| 100 | 7758395CB1 | LUNGDIS03 |
| 101 | 71039312CB1 | BRANDIN01 |
| 102 | 7291318CB1 | BRAIFER06 |
| 103 | 2638619CB1 | COLNFET02 |
| 104 | 2810014CB1 | LUNGTUT17 |
| 105 | 3457155CB1 | THP1NOT03 |
| 106 | 7435171CB1 | PANCDIR02 |
| 107 | 7499936CB1 | PENITUT01 |
| 108 | 7504125CB1 | CONNNOT01 |
| 109 | 7505742CB1 | KIDEUNE02 |
| 110 | 7505757CB1 | THP1NOT03 |
| 111 | 7504126CB1 | SCORNOT04 |
| 112 | 7504099CB1 | KERANOT01 |
| 113 | 7505733CB1 | TESTTUT02 |
| 114 | 7959829CB1 | PROSBPT07 |
| 115 | 7502168CB1 | BRAIUNT01 |
| 116 | 7503888CB1 | NOSEDIC02 |

TABLE 6

| Library | Vector | Library Description |
|---|---|---|
| 293TF5T01 | pINCY | Library was constructed using RNA isolated from a transformed embryonal cell line (293-EBNA) derived from |
| | | kidney epithelial tissue transfected with bgal. The cells were transformed with adenovirus 5 DNA. |
| BRACNOK02 | PSPORT1 | This amplified and normalized library was constructed using RNA isolated from posterior cingulate |
| | | tissue removed from an 85-year-old Caucasian female who died from myocardial infarction and retroperitoneal |
| | | hemorrhage. Pathology indicated atherosclerosis, moderate to severe, involving the circle of Willis, middle |
| | | cerebral, basilar and vertebral arteries; infarction, remote, left dentate nucleus; and amyloid plaque deposition |
| | | consistent with age. There was mild to moderate leptomeningeal fibrosis, especially over the convexity of the |
| | | frontal lobe. There was mild generalized atrophy involving all lobes. The white matter was mildly thinned. |
| | | Cortical thickness in the temporal lobes, both maximal and minimal, was slightly reduced. The substantia nigra pars |
| | | compacta appeared mildly depigmented. Patient history included COPD, hypertension, and recurrent deep venous |
| | | thrombosis. 6.4 million independent clones from this amplified library were normalized in one round using |
| | | conditions adapted from Soares et al., PNAS (1994) 91: 9228-9232 and Bonaldo et al., Genome |
| | | Research 6 (1996): 791. |
| BRAENOT02 | pINCY | Library was constructed using RNA isolated from posterior parietal cortex tissue removed from the brain of a |
| | | 35-year-old Caucasian male who died from cardiac failure. |
| BRAIFEE05 | PCDNA2.1 | This 5′ biased random primed library was constructed using RNA isolated from brain tissue removed from a |
| | | Caucasian male fetus who was stillborn with a hypoplastic left heart at 23 weeks' gestation. |
| BRAIFEN03 | pINCY | This normalized fetal brain tissue library was constructed from 3.26 million independent clones from a fetal |
| | | brain library. Starting RNA was made from brain tissue removed from a Caucasian male fetus, who was stillborn |
| | | with a hypoplastic left heart at 23 weeks' gestation. The library was normalized in 2 rounds using conditions |
| | | adapted from Soares et al., PNAS (1994) 91: 9228 and Bonaldo et al., Genome Research (1996) 6: 791, except that |
| | | a significantly longer (48 hours/round) reannealing hybridization was used. |
| BRAIFER06 | PCDNA2.1 | This random primed library was constructed using RNA isolated from brain tissue removed from a Caucasian male |
| | | fetus who was stillborn with a hypoplastic left heart at 23 weeks' gestation. Serologies were negative. |
| BRAITUT12 | pINCY | Library was constructed using RNA isolated from brain tumor tissue removed from the left frontal lobe of a |
| | | 40-year-old Caucasian female during excision of a cerebral meningeal lesion. Pathology indicated grade 4 |
| | | gemistocytic astrocytoma. |
| BRAIUNT01 | pINCY | Library was constructed using RNA isolated from SK-N-MC, a neuroepithelioma cell line (ATCC HTB-10) derived |
| | | from a 14-year-old Caucasian female with neuroepithelioma, with metastasis to the supra-orbital area. |
| BRANDIN01 | pINCY | This normalized pineal gland tissue library was constructed from .4 million independent clones from a pineal |
| | | gland tissue library from two different donors. Starting RNA was made from pooled pineal gland |
| | | tissue removed from two Caucasian females: a 68-year-old (donor A) who died from congestive heart failure and a |
| | | 79-year old (donor B) who died from pneumonia. Neuropathology for donor A indicated mild to moderate |
| | | Alzheimer disease, atherosclerosis, and multiple infarctions. Neuropathology for donor B indicated severe Alzheimer |
| | | disease, arteriolosclerosis, cerebral amyloid angiopathy and multiple infarctions. There were diffuse and neuritic |
| | | amyloid plaques and neurofibrillary tangles throughout the brain sections examined in both donors. Patient history |
| | | included diabetes mellitus, rheumatoid arthritis, hyperthyroidism, amyloid heart disease, and dementia in donor A; |
| | | and pseudophakia, gastritis with bleeding, glaucoma, peripheral vascular disease, COPD, delayed onset |
| | | tonic/clonic seizures, and transient ischemic attack in donor B. The library was normalized in one round using |
| | | conditions adapted from Soares et al., PNAS (1994) 91: 9228-9232 and Bonaldo et al., Genome Research 6 |
| | | (1996): 791, except that a significantly longer (48 hours/round) reannealing hybridization was used. |
| BRAUNOR01 | pINCY | This random primed library was constructed using RNA isolated from striatum, globus pallidus and posterior |
| | | putamen tissue removed from an 81-year-old Caucasian female who died from a hemorrhage and ruptured |
| | | thoracic aorta due to atherosclerosis. Pathology indicated moderate atherosclerosis involving the internal |
| | | carotids, bilaterally; microscopic infarcts of the frontal cortex and hippocampus, and scattered diffuse amyloid |
| | | plaques and neurofibrillary tangles, consistent with age. Grossly, the leptomeninges showed only mild thickening |
| | | and hyalinization along the superior sagittal sinus. The remainder of the leptomeninges was thin and contained some |
| | | congested blood vessels. Mild atrophy was found mostly in the frontal poles and lobes, and temporal lobes, |
| | | bilaterally. Microscopically, there were pairs of Alzheimer type II astrocytes within the deep layers of the |
| | | neocortex. There was increased satellitosis around neurons in the deep gray matter in the middle frontal cortex. |
| | | The amygdala contained rare diffuse plaques and neurofibrillary tangles. |
| | | The posterior hippocampus contained a microscopic area of cystic cavitation with hemosiderin-laden macrophages |
| | | surrounded by reactive gliosis. Patient history included sepsis, cholangitis, post-operative atelectasis, pneumonia |
| | | CAD, cardiomegaly due to left ventricular hypertrophy, splenomegaly, arteriolonephrosclerosis, nodular colloidal |
| | | goiter, emphysema, CHF, hypothyroidism, and peripheral vascular disease. |
| CERVNOT01 | PSPORT1 | Library was constructed using RNA isolated from the uterine cervical tissue of a 35-year-old Caucasian female |
| | | during a vaginal hysterectomy with dilation and curettage. Pathology indicated mild chronic cervicitis. |
| | | Family history included atherosclerotic coronary artery disease and type II diabetes. |
| COLENOR03 | PCDNA2.1 | Library was constructed using RNA isolated from colon epithelium tissue removed from a 13-year-old Caucasian |
| | | female who died from a motor vehicle accident. |
| COLNFET02 | pINCY | Library was constructed using RNA isolated from the colon tissue of a Caucasian female fetus, who died at 20 |
| | | weeks' gestation. |
| COLNPOT01 | pINCY | Library was constructed using RNA isolated from colon polyp tissue removed from a 40-year-old Caucasian |
| | | female during a total colectomy. Pathology indicated an inflammatory pseudopolyp; this tissue was associated with |
| | | a focally invasive grade 2 adenocarcinoma and multiple tubuvillous adenomas. Patient history included a benign |
| | | neoplasm of the bowel. |
| CONNNOT01 | pINCY | Library was constructed using RNA isolated from mesentery fat tissue obtained from a 71-year-old Caucasian |
| | | male during a partial colectomy and permanent colostomy. Family history included atherosclerotic coronary |
| | | artery disease, myocardial infarction, and extrinsic asthma. |
| CONTTUT01 | pINCY | Library was constructed using RNA isolated from tumorous soft tissue of the left lateral thigh removed from a |
| | | 34-year-old Caucasian female during a soft tissue excision. Pathology indicated metastatic grade 2 myxoid |
| | | liposarcoma which formed multiple, lobulated, circumscribed masses situated in the subcutaneous adipose tissue. |
| | | Patient history included a malignant soft tissue neoplasm of the leg. Family history included benign hypertension, |
| | | acute leukemia, benign hypertension, and type II diabetes. |
| ENDCNOT03 | pINCY | Library was constructed using RNA isolated from dermal microvascular endothelial cells removed from a |
| | | neonatal Caucasian male. |
| EPIPNOT01 | pINCY | Library was constructed using RNA isolated from prostatic epithelial cells removed from a 17-year-old Hispanic |
| | | male. |
| FIBRTXS07 | pINCY | This subtracted library was constructed using 1.3 million clones from a dermal fibroblast library and was |
| | | subjected to two rounds of subtraction hybridization with 2.8 million clones from an untreated dermal fibroblast |
| | | tissue library. The starting library for subtraction was constructed using RNA isolated from treated dermal |
| | | fibroblast tissue removed from the breast of a 31-year-old Caucasian female. The cells were treated with 9CIS |
| | | retinoic acid. The hybridization probe for subtraction was derived from a similarly constructed library from |
| | | RNA isolated from untreated dermal fibroblast tissue from the same donor. Subtractive hybridization conditions |
| | | were based on the methodologies of Swaroop et al., NAR (1991) 19: 1954 and Bonaldo, et al., Genome Research |
| | | (1996) 6: 791. |
| FIBRUNT02 | pINCY | Library was constructed using RNA isolated from an untreated MG-63 cell line derived from an osteosarcoma |
| | | removed from a 14-year-old Caucasian male. |
| GBLATUT01 | pINCY | Library was constructed using RNA isolated from gallbladder tumor tissue removed from a 78-year-old Caucasian |
| | | female during a cholecystectomy. Pathology indicated invasive grade 2 squamous cell carcinoma, forming a mass in |
| | | the gallbladder. Patient history included diverticulitis of the colon, palpitations, benign hypertension, and |
| | | hyperlipidemia. Family history included a cholecystectomy, atherosclerotic coronary artery disease, |
| | | atherosclerotic coronary artery disease, hyperlipidemia, and benign hypertension. |
| HEAONOE01 | PCDNA2.1 | This 5′ biased random primed library was constructed using RNA isolated from the aorta of a 39-year-old |
| | | Caucasian male, who died from a gunshot wound. Serology was positive for cytomegalovirus (CMV). Patient |
| | | history included tobacco abuse (one pack of cigarettes per day for 25 years), and occasionally cocaine, marijuana, |
| | | and alcohol use. |
| HEARNON03 | pINCY | This normalized heart tissue library was constructed from 8.4 million independent clones from a heart tissue |
| | | library. Starting RNA was made from heart tissue removed from a 44-year-old Caucasian male, who died from |
| | | intracranial hemorrhage. Serology was positive for anti-CMV (cytomegalovirus). Patient history included back and |
| | | neck pain, hypertension, pneumonia, sinus infection, alcohol use, and daily pipe tobacco use (×3 years). Patient |
| | | medications included Procardia. The library was normalized in two rounds using conditions adapted from |
| | | Soares et al., PNAS (1994) 91: 9228-9232 and Bonaldo et al., Genome Research (1996) 6: 791, except that |
| | | a significantly longer (48 hours/round) reannealing hybridization was used. |
| HNT2RAT01 | PBLUESCRIPT | Library was constructed at Stratagene (STR937231), using RNA isolated from the hNT2 cell line (derived from a |
| | | human teratocarcinoma that exhibited properties characteristic of a committed neuronal precursor). Cells were |
| | | treated with retinoic acid for 24 hours. |
| KERANOT01 | PBLUESCRIPT | Library was constructed using RNA isolated from neonatal keratinocytes obtained from the leg skin of a |
| | | spontaneously aborted black male. |
| KIDEUNE02 | pINCY | This 5′ biased random primed library was constructed using RNA isolated from an untreated transformed |
| | | embryonal cell line (293-EBNA) derived from kidney epithelial tissue (Invitrogen). The cells were transformed with |
| | | adenovirus 5 DNA. |
| LUNGDIS03 | pINCY | Library was constructed using diseased lung tissue. 0.76 million clones from a diseased lung tissue library |
| | | were subjected to two rounds of subtraction hybridization with 5.1 million clones from a normal lung tissue |
| | | library. The starting library for subtraction was constructed using polyA RNA isolated from diseased lung |
| | | tissue. Patient history included idiopathic pulmonary disease. Subtractive hybridization conditions were based |
| | | on the methodologies of Swaroop et al. (1991) Nucleic Acids Res. 19: 1954; and Bonaldo et al. Genome Res. |
| | | (1996) 6: 791. |
| LUNGTUT17 | pINCY | Library was constructed using RNA isolated from lung tumor tissue removed from a 53-year-old male. Pathology |
| | | indicated grade 4 adenocarcinoma. |
| LUNLTUT11 | pINCY | Library was constructed using RNA isolated from lung tumor tissue removed from the right upper lobe of a |
| | | 50-year-old Caucasian male during segmental lung resection. Pathology indicated an invasive grade 4 squamous cell |
| | | adenocarcinoma forming a subpleural mass, which puckered the underlying pleura. The tumor did not infiltrate |
| | | the pleura. Reactive mesothelial cells and fibrin were present at the right lower lobe of pleural implant. |
| | | Patient history included a respiratory anomaly, chest pain, and tobacco abuse. Family history included skin cancer |
| | | and type II diabetes. |
| MCLDTXN05 | pINCY | This normalized dendritic cell library was constructed from 1 million independent clones from a pool of two derived |
| | | dendritic cell libraries. Starting libraries were constructed using RNA isolated from untreated and treated derived |
| | | dendritic cells from umbilical cord blood CD34+ precursor cells removed from a male. The cells were derived |
| | | with granulocyte/macrophage colony stimulating factor (GM-CSF), tumor necrosis factor alpha (TNF alpha), and |
| | | stem cell factor (SCF). The GM-CSF was added at time 0 at 100 ng/ml the TNF alpha was added at time 0 at |
| | | 2.5 ng/ml, and the SCF was added at time 0 at 25 ng/ml. Incubation time was 13 days. The treated cells were |
| | | then exposed to phorbol myristate acetate (PMA), and Ionomycin. The PMA and Ionomycin were added at 13 days |
| | | for five hours. The library was normalized in two rounds using conditions adapted from Soares et al., PNAS |
| | | (1994) 91: 9228 and Bonaldo et al., Genome Research 6 (1996): 791, except that a significantly longer |
| | | (48 hours/round) reannealing hybridization was used. |
| MMLR3DT01 | PSPORT1 | Library was constructed using RNA isolated from adherent mononuclear cells, which came from a pool of |
| | | male and female donors. |
| MUSCNOT10 | pINCY | Library was constructed using RNA isolated from gluteal muscle tissue removed from a 43-year-old Caucasian |
| | | female during soft tissue excision, partial ostectomy, and plastic skin repair. Pathology for the associated |
| | | tumor tissue indicated recurrent clear cell sarcoma of soft parts, forming a mass in the coccygeal region, |
| | | associated with a cystic cavity (previous biopsy site). Family history included benign hypertension, |
| | | osteoarthritis, prostate cancer, depression, osteoarthritis, benign hypertension, colon cancer, and depression. |
| NERDTDN03 | pINCY | This normalized dorsal root ganglion tissue library was constructed from 1.05 million independent |
| | | clones from a dorsal root ganglion tissue library. Starting RNA was made from dorsal root ganglion tissue removed |
| | | from the cervical spine of a 32-year-old Caucasian male who died from acute pulmonary edema, |
| | | acute bronchopneumonia, bilateral pleural effusions, pericardial effusion, and malignant lymphoma (natural killer |
| | | cell type). The patient presented with pyrexia of unknown origin, malaise, fatigue, and gastrointestinal bleeding. |
| | | Patient history included probable cytomegalovirus infection, liver congestion, and steatosis, splenomegaly, |
| | | hemorrhagic cystitis, thyroid hemorrhage, respiratory failure, pneumonia of the left lung, natural killer cell |
| | | lymphoma of the pharynx, Bell's palsy, and tobacco and alcohol abuse. Previous surgeries included colonoscopy, |
| | | closed colon biopsy, adenotonsillectomy, and nasopharyngeal endoscopy and biopsy. Patient medications included |
| | | Diflucan (fluconazole), Deltasone (prednisone), hydrocodone, Lortab, Alprazolam, Reazodone, ProMace-Cytabom, |
| | | Etoposide, Cisplatin, Cytarabine, and dexamethasone. |
| | | The patient received radiation therapy and multiple blood transfusions. The library was normalized in 2 rounds using |
| | | conditions adapted from Soares et al., PNAS (1994) 91: 9228-9232 and Bonaldo et al., Genome Research 6 (1996): |
| | | 791, except that a significantly longer (48 hours/round) reannealing hybridization was used. |
| NOSEDIC02 | PSPORT1 | This large size fractionated library was constructed using RNA isolated from nasal polyp tissue. |
| OVARDIJ01 | pIGEN | This random primed 5′ cap isolated library was constructed using RNA isolated from diseased right ovary |
| | | tissue removed from a 47-year-old Caucasian female during total abdominal hysterectomy, dilation and curettage, |
| | | bilateral salpingo-oophorectomy, repair of ureter, and incidental appendectomy. Pathology indicated endometriosis. |
| | | Pathology for the associated tumor tissue indicated multiple leiomyomata. The left ovary contained a corpus luteum. |
| | | There was endometriosis involving the posterior serosa. The patient presented with metrorrhagia and a benign |
| | | neoplasm of the ovary. Patient history included normal delivery, joint pain in multiple joints, and unilateral |
| | | congenital hip dislocation. Previous surgeries included total hip replacement. Patient medications included calcium. |
| | | Family history included kidney cancer in the mother; atherosclerotic coronary artery disease and aortocoronary |
| | | bypass of 3 coronary arteries in the father; benign hypertension and Hodgkin's disease in the sibling(s); and |
| | | benign hypertension and cerebrovascular accident in the grandparent(s). |
| PANCDIR02 | PCDNA2.1 | This random primed library was constructed using RNA isolated from diseased pancreatic tissue removed |
| | | from a 43-year-old Caucasian female who died from a gunshot wound to the head. Patient history included type I |
| | | diabetes for 38 years, a fractured finger, and tobacco use (1 pack per day for 25 years). The serology was positive |
| | | CMV antibody and remaining serologies were negative. Patient medications included antidepressants and Insulin. |
| PANCNOE02 | PCDNA2.1 | This 5′ biased random primed library was constructed using RNA isolated from pancreatic tissue removed |
| | | from an 8-year-old Black male, who died from anoxia. Serologies were negative. Patient medications included |
| | | DDAVP, Versed, and labetalol. |
| PANCNOT04 | PSPORT1 | Library was constructed using RNA isolated from the pancreatic tissue of a 5-year-old Caucasian male who |
| | | died in a motor vehicle accident. |
| PANCNOT05 | PSPORT1 | Library was constructed using RNA isolated from the pancreatic tissue of a 2-year-old Hispanic male who |
| | | died from cerebral anoxia. |
| PANCTUT02 | pINCY | Library was constructed using RNA isolated from pancreatic tumor tissue removed from a 45-year-old |
| | | Caucasian female during radical pancreaticoduodenectomy. Pathology indicated a grade 4 anaplastic carcinoma. |
| | | Family history included benign hypertension, hyperlipidemia and atherosclerotic coronary artery disease. |
| PENITUT01 | pINCY | Library was constructed using RNA isolated from tumor tissue removed from the penis of a 64-year-old |
| | | Caucasian male during penile amputation. Pathology indicated a fungating invasive grade 4 squamous cell |
| | | carcinoma involving the inner wall of the foreskin and extending onto the glans penis. Patient history included |
| | | benign neoplasm of the large bowel, atherosclerotic coronary artery disease, angina pectoris, gout, and obesity. |
| | | Family history included malignant pharyngeal neoplasm, chronic lymphocytic leukemia, and chronic liver disease. |
| PROSBPT07 | pINCY | Library was constructed using RNA isolated from diseased prostate tissue removed from a 53-year-old Caucasian |
| | | male during radical prostatectomy and regional lymph node excision. Pathology indicated adenofibromatous |
| | | hyperplasia. Pathology for the associated tumor tissue indicated adenocarcinoma (Gleason grade 3 + 2). |
| | | The patient presented with elevated prostate specific antigen and induration. Patient history included |
| | | hyperlipidemia. Family history included atherosclerotic coronary artery disease, coronary artery bypass |
| | | graft, perforated gallbladder, hyperlipidemia, and kidney stones. |
| PROSTME06 | PCDNA2.1 | This 5′ biased random primed library was constructed using RNA isolated from diseased prostate tissue removed |
| | | from a 57-year-old Caucasian male during closed prostatic biopsy, radical prostatectomy, and regional lymph |
| | | node excision. Pathology indicated adenofibromatous hyperplasia. Pathology for the matched tumor tissue |
| | | indicated adenocarcinoma, Gleason grade 3 + 3, forming a predominant mass involving the right side centrally. |
| | | The patient presented with elevated prostate specific antigen and prostate cancer. Patient history |
| | | included tobacco abuse in remission. Previous surgeries included cholecystectomy, repair of diaphragm hernia, and |
| | | repair of vertebral fracture. Patient medications included Pepsid, Omnipen, and Eulexin. Family history included |
| | | benign hypertension, cerebrovascular accident, atherosclerotic coronary artery disease, uterine cancer and type II |
| | | diabetes in the mother; prostate cancer in the father; drug abuse, prostate cancer, and breast cancer in the |
| | | sibling(s). |
| SCORNON02 | PSPORT1 | This normalized spinal cord library was constructed from 3.24M independent clones from the a spinal cord |
| | | tissue library. RNA was isolated from the spinal cord tissue removed from a 71-year-old Caucasian male who |
| | | died from respiratory arrest. Patient history included myocardial infarction, gangrene, and end stage renal disease. |
| | | The normalization and hybridization conditions were adapted from Soares et al. (PNAS (1994) 91: 9228). |
| SCORNOT04 | pINCY | Library was constructed using RNA isolated from cervical spinal cord tissue removed from a 32-year-old |
| | | Caucasian male who died from acute pulmonary edema and bronchopneumonia, bilateral pleural and pericardial |
| | | effusions, and malignant lymphoma (natural killer cell type). Patient history included probable cytomegalovirus |
| | | infection, hepatic congestion and steatosis, splenomegaly, hemorrhagic cystitis, thyroid hemorrhage, and |
| | | Bell's palsy. Surgeries included colonoscopy, large intestine biopsy, adenotonsillectomy, and nasopharyngeal |
| | | endoscopy and biopsy; treatment included radiation therapy. |
| TESTNON04 | pINCY | This normalized testis tissue library was constructed from 6.48 million independent clones from |
| | | a pool of testis tissue libraries. Starting RNA was made from testicular tissue removed from a 16-year-old |
| | | Caucasian male who died from hanging. The library was normalized in two rounds using conditions adapted |
| | | from Soares et al., PNAS (1994) 91: 9228 and Bonaldo et al., Genome Research 6 (1996): 791, except that |
| | | a significantly longer (48-hours/round)reannealing hybridization was used. |
| TESTTUT02 | pINCY | Library was constructed using RNA isolated from testicular tumor removed from a 31-year-old Caucasian |
| | | male during unilateral orchiectomy. Pathology indicated embryonal carcinoma. |
| THP1NOT03 | pINCY | Library was constructed using RNA isolated from untreated THP-1 cells. THP-1 is a human promonocyte |
| | | line derived from the peripheral blood of a 1-year-old Caucasian male with acute monocytic leukemia (ref: |
| | | Int. J. Cancer (1980) 26: 171). |
| THYMNOE02 | PCDNA2.1 | This 5′ biased random primed library was constructed using RNA isolated from thymus tissue removed |
| | | from a 3-year-old Hispanic male during a thymectomy and closure of a patent ductus arteriosus. The patient |
| | | presented with severe pulmonary stenosis and cyanosis. Patient history included a cardiac catheterization and |
| | | echocardiogram. Previous surgeries included Blalock-Taussig shunt and pulmonary valvotomy. The patient |
| | | was not taking any medications. Family history included benign hypertension, osteoarthritis, depressive |
| | | disorder, and extrinsic asthma in the grandparent(s). |
| THYRDIE01 | PCDNA2.1 | This 5′ biased random primed library was constructed using RNA isolated from diseased thyroid tissue removed |
| | | from a 22-year-old Caucasian female during closed thyroid biopsy, partial thyroidectomy, and regional |
| | | lymph node excision. Pathology indicated adenomatous hyperplasia. The patient presented with malignant neoplasm |
| | | of the thyroid. Patient history included normal delivery, alcohol abuse, and tobacco abuse. Previous surgeries |
| | | included myringotomy. Patient medications included an unspecified type of birth control pills. Family history |
| | | included hyperlipidemia and depressive disorder in the mother; and benign hypertension, congestive heart failure, |
| | | and chronic leukemia in the grandparent(s). |
| UTRCDIE01 | PCDNA2.1 | This 5′ biased random primed library was constructed using RNA isolated from uterine cervix tissue |
| | | removed from a 29-year-old Caucasian female during a vaginal hysterectomy and cystocele repair. Pathology |
| | | indicated the cervix showed mild chronic cervicitis with focal squamous metaplasia. Pathology for the matched |
| | | tumor tissue indicated intramural uterine leiomyoma. Patient history included hypothyroidism, pelvic |
| | | floor relaxation, paraplegia, and self catheterization. Previous surgeries included a normal delivery, a |
| | | laminectomy, and a rhinoplasty. Patient medications included Synthroid. Family history included benign |
| | | hypertension in the father; and type II diabetes and hyperlipidemia in the mother. |
| UTRSDIC01 | PSPORT1 | This large size fractionated library was constructed using pooled cDNA from eight donors. cDNA was generated |
| | | using mRNA isolated from endometrial tissue removed from a 32-year-old female (donor A); endometrial tissue |
| | | removed from a 32-year-old Caucasian female (donor B) during abdominal hysterectomy, bilateral |
| | | salpingo-oophorectomy, and cystocele repair; from diseased endometrium and myometrium tissue removed |
| | | from a 38-year-old Caucasian female (donor C) during abdominal hysterectomy, bilateral salpingo-oophorectomy, |
| | | and exploratory laparotomy; from endometrial tissue removed from a 41-year-old Caucasian female (donor D) |
| | | during abdominal hysterectomy with removal of a solitary ovary; from endometrial tissue removed from a |
| | | 43-year-old Caucasian female (donor E) during vaginal hysterectomy, dilation and curettage, cystocele repair, |
| | | rectocele repair and cystostomy; and from endometrial tissue removed from a 48-year-old Caucasian female |
| | | (donor F) during a vaginal hysterectomy, rectocele repair, and bilateral salpingo-oophorectomy. Pathology (A) |
| | | indicated the endometrium was in secretory phase. Pathology (B) indicated the endometrium was in the proliferative |
| | | phase. Pathology (C) indicated extensive adenomatous hyperplasia with squamous metaplasia and focal atypia, |
| | | forming a polypoid mass within the endometrial cavity. The cervix showed chronic cervicitis and squamous |
| | | metaplasia. Pathology (D, E) indicated the endometrium was secretory phase. Pathology (F) indicated the |
| | | endometrium was weakly proliferative. |
| UTRSNOT02 | PSPORT1 | Library was constructed using RNA isolated from uterine tissue removed from a 34-year-old Caucasian female |
| | | during a vaginal hysterectomy. Patient history included mitral valve disorder. Family history included |
| | | stomach cancer, congenital heart anomaly, irritable bowel syndrome, ulcerative colitis, colon cancer, |
| | | cerebrovascular disease, type II diabetes, and depression. |
| UTRSTMR01 | pINCY | Library was constructed using RNA isolated from uterine myometrial tissue removed from a 41-year-old |
| | | Caucasian female during a vaginal hysterectomy. The endometrium was secretory and contained fragments of |
| | | endometrial polyps. Pathology for associated tumor tissue indicated uterine leiomyoma. Patient history |
| | | included ventral hernia and a benign ovarian neoplasm. |

TABLE 7

| Program | Description | Reference | Parameter Threshold |
|---|---|---|---|
| ABI | A program that removes vector | Applied Biosystems, Foster City, CA. | |
| FACTURA | sequences and masks ambiguous | | |
| | bases in nucleic acid sequences. | | |
| ABI/ | A Fast Data Finder useful in | Applied Biosystems, Foster City, CA; | Mismatch <50% |
| PARACEL | comparing and annotating amino | Paracel Inc., Pasadena, CA. | |
| FDF | acid or nucleic acid sequences. | | |
| ABI | A program that assembles | Applied Biosystems, Foster City, CA. | |
| AutoAssembler | nucleic acid sequences. | | |
| BLAST | A Basic Local Alignment Search | Altschul, S. F. et al. (1990) J. Mol. Biol. | ESTs: Probaility value = 1.0E−8 |
| | Tool useful in sequence | 215: 403-410; Altschul, S. F. et al. (1997) | or less |
| | similarity search for amino | Nucleic Acids Res. 25: 3389-3402. | Full Length sequences: Probability |
| | acid and nucleic acid sequences. | | value = 1.0E−10 or less |
| | BLAST includes five functions: | | |
| | blastp, blastn, blastx, | | |
| | tblastn, and tblastx. | | |
| FASTA | A Pearson and Lipman | Pearson, W. R. and D. J. Lipman (1988) Proc. | ESTs: fasta E value = 1.06E−6 |
| | algorithm that searches for | Natl. Acad Sci. USA 85: 2444-2448; Pearson, W. R. | Assembled ESTs: fasta Identity = 95% |
| | similarity between a query | (1990) Methods Enzymol. 183: 63-98; | or greater and |
| | sequence and a group of | and Smith, T. F. and M. S. Waterman (1981) | Match length = 200 bases or greater; |
| | sequences of the same type. | Adv. Appl. Math. 2: 482-489. | fastx E value = 1.0E−8 or less |
| | FASTA comprises as least five | | Full Length sequences: |
| | functions: fasta, tfasta, fastx, | | fastx score = 100 or greater |
| | tfastx, and ssearch. | | |
| BLIMPS | A BLocks IMProved Searcher | Henikoff, S. and J. G. Henikoff (1991) Nucleic | Probability value = 1.0E−3 or less |
| | that matches a sequence against | Acids Res. 19: 6565-6572; Henikoff, J. G. & S. Henikoff | |
| | those in BLOCKS, PRINTS, | (1996) Methods Enzymol. 266: 88-105; | |
| | DOMO, PRODOM, and PFAM | and Attwood, T. K. et al. (1997) J. Chem. | |
| | databases to search for gene | Inf. Comput. Sci. 37: 417-424. | |
| | families, sequence homology, | | |
| | and structural fingerprint | | |
| | regions. | | |
| HMMER | An algorithm for searching a | Krogh, A. et al. (1994) J. Mol. Biol. 235: 1501-1531; | PFAM, INCY, SMART, or TIGRFAM |
| | query sequence against hidden | Sonnhammer, E. L. L. et al. (1988) | hits: Probability value = 1.0E−3 or less |
| | Markov model (HMM)-based | Nucleic Acids Res. 26: 320-322; Durbin, R. et | Signal peptide hits: Score = 0 or |
| | databases of protein family | al. (1998) Our World View, in a Nutshell, | greater |
| | consensus sequences, such as | Cambridge Univ. Press, p. 1-350 | |
| | PFAM, INCY, SMART, and | | |
| | TIGRFAM. | | |
| ProfileScan | An algorithm that searches | Gribskov, M. et al. (1988) CABIOS 4: 61-66; | Normalized quality score ≧ GCG- |
| | for structural and sequence | Gribskov, M. et al. (1989) Methods Enzymol. | specified “HIGH” value for that |
| | motifs in protein sequences | 183: 146-159; Bairoch, A. et al. (1997) | particular Prosite motif. |
| | that match sequence patterns | Nucleic Acids Res. 25: 217-221. | Generally, score = 1.4-2.1. |
| | defined in Prosite. | | |
| Phred | A base-calling algorithm that | Ewing, B. et al. (1998) Genome Res. | |
| | examines automated sequencer | 8: 175-185; Ewing, B. and P. Green | |
| | traces with high sensitivity | (1998) Genome Res. 8: 186-194. | |
| | and probability. | | |
| Phrap | A Phils Revised Assembly | Smith, T. F. and M. S. Waterman (1981) Adv. | Score = 120 or greater; |
| | Program including SWAT and | Appl. Math. 2: 482-489; Smith, T. F. and M. S. Waterman | Match length = 56 or greater |
| | CrossMatch, programs based | (1981) J. Mol. Biol. 147: 195-197; | |
| | on efficient implementation | and Green, P., University of Washington, | |
| | of the Smith-Waterman | Seattle, WA. | |
| | algorithm, useful in searching | | |
| | sequence homology and | | |
| | assembling DNA sequences. | | |
| Consed | A graphical tool for viewing and | Gordon, D. et al. (1998) Genome Res. 8: 195-202. | |
| | editing Phrap assemblies. | | |
| SPScan | A weight matrix analysis | Nielson, H. et al. (1997) Protein Engineering | Score = 3.5 or greater |
| | program that scans protein | 10: 1-6; Claverie, J. M. and S. Audic (1997) | |
| | sequences for the presence | CABIOS 12: 431-439. | |
| | of secretory signal peptides. | | |
| TMAP | A program that uses weight | Persson, B. and P. Argos (1994) J. Mol. Biol. | |
| | matrices to delineate | 237: 182-192; Persson, B. and P. Argos (1996) | |
| | transmembrane segments on | Protein Sci. 5: 363-371. | |
| | protein sequences and | | |
| | determine orientation. | | |
| TMHMMER | A program that uses a hidden | Sonnhammer, E. L. et al. (1998) Proc. Sixth Intl. | |
| | Markov model (HMM) to | Conf. on Intelligent Systems for Mol. Biol., | |
| | delineate transmembrane | Glasgow et al., eds., The Am. Assoc. for Artificial | |
| | segments on protein sequences | Intelligence Press, Menlo Park, CA, pp. 175-182. | |
| | and determine orientation. | | |
| Motifs | A program that searches amino | Bairoch, A. et al. (1997) Nucleic Acids Res. 25: 217-221; | |
| | acid sequences for patterns that | Wisconsin Package Program Manual, version 9, page | |
| | matched those defined in | M51-59, Genetics Computer Group, Madison, WI. | |
| | Prosite. | | |

1. An isolated polypeptide selected from the group consisting of:
a) a polypeptide comprising an amino acid sequence selected from the group consisting of SEQ ID NO:1-17, SEQ ID NO:23-25, and SEQ ID NO:28-58,
b) a polypeptide consisting essentially of a naturally occurring amino acid sequence selected from the group consisting of SEQ ID NO: 18-22 and SEQ ID NO:26-27,
c) a polypeptide consisting essentially of a naturally occurring amino acid sequence at least 90% identical to an amino acid sequence consisting of SEQ ID NO: 1, SEQ ID NO:3-5, SEQ ID NO:9, SEQ ID NO:15, SEQ ID NO:18-22, SEQ ID NO:26-27, SEQ ID NO:35-36, SEQ ID NO:41, SEQ ID NO:49-50, SEQ ID NO:53, and SEQ ID NO:58,
d) a polypeptide comprising a naturally occurring amino acid sequence at least 90% identical to an amino acid sequence selected from the group consisting of SEQ ID NO:2, SEQ ID NO:6, SEQ ID NO: 10-11, SEQ ID NO:13, SEQ ID NO:16, SEQ ID NO:28-29, SEQ ID NO:31-34, SEQ ID NO:39-40, SEQ ID NO:42-43, SEQ ID NO:46, SEQ ID NO:52, and SEQ ID NO:57,
e) a polypeptide comprising a naturally occurring amino acid sequence at least 91% identical to the amino acid sequence of SEQ ID NO:47,
f) a polypeptide comprising a naturally occurring amino acid sequence at least 92% identical to an amino acid sequence selected from the group consisting of SEQ ID NO:23, and SEQ ID NO:38,
g) a polypeptide comprising a naturally occurring amino acid sequence at least 93% identical to the amino acid sequence of SEQ ID NO:55,
h) a polypeptide comprising a naturally occurring amino acid sequence at least 94% identical to the amino acid sequence of SEQ ID NO:24,
i) a polypeptide comprising a naturally occurring amino acid sequence at least 95% identical to an amino acid sequence selected from the group consisting of SEQ ID NO:12, SEQ ID NO: 14, SEQ ID NO:37, and SEQ ID NO:56,
j) a polypeptide comprising a naturally occurring amino acid sequence at least 96% identical to an amino acid sequence selected from the group consisting of SEQ ID NO:8, SEQ ID NO: 17, and SEQ ID NO:48,
k) a polypeptide comprising a naturally occurring amino acid sequence at least 97% identical to an amino acid sequence selected from the group consisting of SEQ ID NO:30, and SEQ ID NO:45,
l) a polypeptide comprising a naturally occurring amino acid sequence at least 99% identical to an amino acid sequence selected from the group consisting of SEQ ID NO:25, SEQ ID NO:44, SEQ ID NO:51, and SEQ ID NO:54,
m) a biologically active fragment of a polypeptide having an amino acid sequence selected from the group consisting of SEQ ID NO: 1-58, and
n) an immunogenic fragment of a polypeptide having an amino acid sequence selected from the group consisting of SEQ ID NO:1-58.

2. An isolated polypeptide of claim 1 selected from the group consisting of:
a) a polypeptide comprising an amino acid sequence selected from the group consisting of SEQ ID NO: 1-17, SEQ ID NO 23-25, and SEQ ID NO:28-58, and
b) a polypeptide consisting essentially of an amino acid sequence selected from the group consisting of SEQ ID NO:18-22, and SEQ ID NO:26-27.

3. An isolated polynucleotide encoding a polypeptide of claim 1.

4. An isolated polynucleotide encoding a polypeptide of claim 2.

5. An isolated polynucleotide of claim 4 comprising a polynucleotide sequence selected from the group consisting of SEQ ID NO:59-116.

6. A recombinant polynucleotide comprising a promoter sequence operably linked to a polynucleotide of claim 3.

7. A cell transformed with a recombinant polynucleotide of claim 6.

8. (CANCELED)

9. A method of producing a polypeptide of claim 1, the method comprising:
a) culturing a cell under conditions suitable for expression of the polypeptide, wherein said cell is transformed with a recombinant polynucleotide, and said recombinant polynucleotide comprises a promoter sequence operably linked to a polynucleotide encoding the polypeptide of claim 1, and
b) recovering the polypeptide so expressed.

10. A method of claim 9, wherein the polypeptide comprises an amino acid sequence selected from the group consisting of SEQ ID NO: 1-58.

11. An isolated antibody which specifically binds to a polypeptide of claim 1.

12. An isolated polynucleotide selected from the group consisting of:
a) a polynucleotide comprising a polynucleotide sequence selected from the group consisting of SEQ ID NO:59-116,
b) a polynucleotide comprising a naturally occurring polynucleotide sequence at least 90% identical to a polynucleotide sequence selected from the group consisting of SEQ ID NO:59-108 and SEQ ID NO:110-116,
c) a polynucleotide comprising a naturally occurring polynucleotide sequence at least 97% identical to the polynucleotide sequence of SEQ ID NO: 109,
d) a polynucleotide complementary to a polynucleotide of a),
e) a polynucleotide complementary to a polynucleotide of b),
f) a polynucleotide complementary to a polynucleotide of c), and
g) an RNA equivalent of a)-f).

13. (CANCELED)

14. A method of detecting a target polynucleotide in a sample, said target polynucleotide having a sequence of a polynucleotide of claim 12, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

15. (CANCELED)

16. A method of detecting a target polynucleotide in a sample, said target polynucleotide having a sequence of a polynucleotide of claim 12, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

17. A composition comprising a polypeptide of claim 1 and a pharmaceutically acceptable excipient.

18. A composition of claim 17, wherein the polypeptide is selected from the group consisting of:
a) a polypeptide comprising an amino acid sequence selected from the group consisting of SEQ ID NO:1-17, SEQ ID NO:23-25, and SEQ ID NO:28-58, and
b) a polypeptide consisting essentially of an amino acid sequence selected from the group consisting of SEQ ID NO:18-22, and SEQ ID NO:26-27.

19. (CANCELED)

20. A method of screening a compound for effectiveness as an agonist of a polypeptide of claim 1, the method comprising:
a) exposing a sample comprising a polypeptide of claim 1 to a compound, and
b) detecting agonist activity in the sample.

21. (CANCELED)

22. (CANCELED)

23. A method of screening a compound for effectiveness as an antagonist of a polypeptide of claim 1, the method comprising:
a) exposing a sample comprising a polypeptide of claim 1 to a compound, and
b) detecting antagonist activity in the sample.

24. (CANCELED)

25. (CANCELED)

26. A method of screening for a compound that specifically binds to the polypeptide of claim 1, the method comprising:
a) combining the polypeptide of claim 1 with at least one test compound under suitable conditions, and
b) detecting binding of the polypeptide of claim 1 to the test compound, thereby identifying a compound that specifically binds to the polypeptide of claim 1.

27. (CANCELED)

28. A method of screening a compound for effectiveness in altering expression of a target polynucleotide, wherein said target polynucleotide comprises a sequence of claim 5, the method comprising:
a) exposing a sample comprising the target polynucleotide to a compound, under conditions suitable for the expression of the target polynucleotide,
b) detecting altered expression of the target polynucleotide, and
c) comparing the expression of the target polynucleotide in the presence of varying amounts of the compound and in the absence of the compound.

29. A method of assessing toxicity of a test compound, the method comprising:
a) treating a biological sample containing nucleic acids with the test compound,
b) hybridizing the nucleic acids of the treated biological sample with a probe comprising at least 20 contiguous nucleotides of a polynucleotide of claim 12 under conditions whereby a specific hybridization complex is formed between said probe and a target polynucleotide in the biological sample, said target polynucleotide comprising a polynucleotide sequence of a polynucleotide of claim 12 or fragment thereof,
c) quantifying the amount of hybridization complex, and
d) comparing the amount of hybridization complex in the treated biological sample with the amount of hybridization complex in an untreated biological sample, wherein a difference in the amount of hybridization complex in the treated biological sample is indicative of toxicity of the test compound.

30-171. (canceled)


SEQUENCE LISTING (116 sequences)

SEQ ID NO: 1 - protein, 1374 aa, Homo sapiens
  misc_feature: Incyte ID No 7503848CD1
MAEARKRRELLPLIYHHLLRAGYVRAAREVKEQSGQKCFLAQPVTLLDIYTHWQQTSELGRKRKAEEDAALQAKKTRVSDPISTSESSEEEEEAEAETAKATPRLASTNSSVLGADLPSSMKEKAKAETEKAGKTGNSMPHPATGKTVANLLSGKSPRKSAEPSANTTLVSETEEEGSVPAFGAAAKPGMVSAGQADSSSEDTSSSSDETDVEVKASEKILQVRAASAPAKGTPGKGATPAPPGKAGAVASQTKAGKPEEDSESSSEESSDSEEETPAAKALLQAKASGKTSQVGAASAPAKESPRKGAAPAPPGKTGPAVAKAQAGKREEDSQSSSEESDSEEEAPAQAKPSGKAPQVRAASAPAKESPRKGAAPAPPRKTGPAAAQVQVGKQEEDSRSSSEESDSDREALAAMNAAQVKPLGKSPQVKPASTMGMGPLGKGAGPVPPGKVGPATPSAQVGKWEEDSESSSEESSDSSDGEVPTAVAPAQEKSLGNILQAKPTSSPAKGPPQKAGPVAVQVKAEKPMDNSESSEESSDSADSEEAPAAMTAAQAKPALKIPQTKACPKKTNTTASAKVAPVRVGTQAPRKAGTATSPAGSSPAVAGGTQRPAEDSSSSEESDSEEEKTGLAVTVGQAKSVGKGLQVKAASVPVKGSLGQGTAPVLPGKTGPTVTQVKAEKQEDSESSEEESDSEEAAASPAQVKTSVKKTQAKANPAAARAPSAKGTISAPGKVVTAAAQAKQRSPSKVKPPVRNPQNSTVLARGPASVPSVGKAVATAAQAQTGPEEDSGSSEEESDSEEEAETLAQVKPSGKTHQIRAALAPAKESPRKGAAPTPPGKTGPSAAQAGKQDDSGSSSEESDSDGEAPAAVTSAQVIKPPLIFVDPNRSPAGPAATPAQAQAASTPRKARASESTARSSSSESEDEDVIPATQCLTPGIRTNVVTMPTAHPRIAPKASMAGASSSKESSRISDGKKQEGPATQVDSAVGTLPATSPQSTSVQAKGTNKLRKPKLPEVQQATKAPESSDDSEDSSDSSSGSEEDGEGPQGAKSAHTLVGPTPSRTETLVEETAAESSEDDVVAPSQSLLSGYMTPGLTPANSQASKATPKLDSSPSVSSTLAAKDDPDGKQEAKPQQAAGMLSPKTGGKEAASGTTPQKSRKPKKGAGNPQASTLALQSNITQCLLGQPWPLNEAQVQASVVKVLTELLEQERKKVVDTTKESSRKGWESRKRKLSGDQPAARTPRSKKKKKLGAGEGGEASVSPEKTSTTSKGKAKRDKASGDVKEKKGKGSLGSQGAKDEPEEELQKGMGTVEGGDQSNPKSKKEKKKSDKRKKDKEKKEKKKKAKKASTKDSESPSQKKKKKKKKTAEQTV

SEQ ID NO: 2 - protein, 588 aa, Homo sapiens
  misc_feature: Incyte ID No 2608080CD1
MAAPALALVSFENVVVTFTGEEWGHLDLAQRTLYQEVMLETCRLLVSLGHPVPKPELIYLLEHGQELWTVKRGLSQSTCAGEKAKPKITEPTASQLAFSEESSFQELLAQRSSRDSRLGQARDEEKLIKIQEGNLRPGTNPHKEICPEKLSYKHDDLEPDDSLGLRVLQERVTPQDALHECDSQGPGKDPMTDARNNPYTCTECGKGFSKKWALVRHQQIHAGVKPYECNECGKACRYMADVIRHMRLHTGEKPYKCIECGKAFKRRFHLTEHQRIHTGDKPYECKECGKAFTHRSSFIQHNMTHTREKPFLCKECGKAFYYSSSFAQHMRIHTGKKLYECGECGKAFTHRSTFIQHNVTHTGEKPFLCKECGKTFCLNSSFTQHMRIHTGEKPYECGECGKAFTHRSTFIRHKRTHTGEKPFECKECGKAFCDSSSLIQHMRIHTGEKPYECSECGKAFTHHSVFIRHNRTHSGQKPLECKECAKAFYYSSSFTRHMRIHTGEKPYVCRECGKAFTQPANFVRHNRIHTGEKPFECKECEKAFCDNFALTQHMRTHTGEKPFECNECGKTFSHSSSFTHHRKIHTRV

SEQ ID NO: 3 - protein, 607 aa, Homo sapiens [sequence omitted]

SEQ ID NO: 4 - protein, 422 aa, Homo sapiens
  misc_feature: Incyte ID No 7503517CD1
MEFQAVVMAVGGGSRMTDLTSSIPKPLLPVGNKPLIWYPLNLLERVGFEEVIVVTTRDVQKALCAEFKMKMKPDIVCIPDDADMGTADSLRYIYPKLKTDVLVLSCDLITDVALHEVVDLFRAYDASLAMLMRKGQDSIEPVPGQKGKKKAVEQRDFIGVDSTGKRLLFMANEADLDEELVIKGSILQKSITSIRSELIPYLVRKQFSSASSQQGQEEKEEDLKKKELKSLDIYSFIKEANTLNLAPYDACWNACRGDRWEDLSRSQVRCYVHIMKEGLCSRVSTLGLYMEANRQVPKLLSALCPEEPPVHSSAQIVSKHLVGVDSLIGPETQIGEKSSIKRSVIGSSCLIKDRVTITNCLLMNSVTVEEGSNIQGSVICNNAVIEKGADIKDCLIGSGQRIEAKAKRVNEVIVGNDQLMEI

SEQ ID NO: 5 - protein, 142 aa, Homo sapiens
  misc_feature: Incyte ID No 7500014CD1
MSEGESQTVLSSGSDPKVESSSSAPGLTSPVVPPSVKTPTPEPAEVETRKVVLMQCNIESVEEGVKHHLTLLLKLEDKLNRHLSCDLMPNENIPELAAELVQLGFISEADQSRLTSLLEETLNKFNFARNSTLNSAAVTVSS

SEQ ID NO: 6 - protein, 433 aa, Homo sapiens
  misc_feature: Incyte ID No 7501365CD1
MARVAWGLLWLLLGSAGAQYEKYSFRGFPPEDLMPLAAAYGHALEQYEGESWRESARYLEAALRLHRLLRDSEAFCHANCSGPAPAAKPDPDGGRADEWACELRLFGRVLERAACLRRCKRTLPAFQVPYPPRQLLRDFQSRLPYQYLHYALFKANRLEKAVAAAYTFLQRNPKHELTAKYLNYYRGMLDVADESLTDLEAQPYEAVFLRAVKLYNSGDFRSSTEDMERALSEYLAVFARCLAGCEGAHEQVDFKDFYPAIADLFAESLQCKVDCEANLTPNVGGYFVDKFVATMYHYLQFAYYKLNDVRQAARSAASYMLFDPKDSVMQQNLVYYRFHRARWGLEEEDFQPREEAMLYHNQTAELRELLEFTHMYLQSDDESQSLNSHEKGTPHTPQAWEAWCRWPHPHQPGQQQELFIKNLRWARCGGSHL

SEQ ID NO: 7 - protein, 1450 aa, Homo sapiens
  misc_feature: Incyte ID No 7503540CD1
MSLTSWFLVSSGGTRHRLPREMIFVGRDDCELMLQSRSVDKQHAVINYDASTDEHLVKDLGSLNGTFVNDVRIPEQTYITLKLEDKLRFGYDTNLFTVVQGEMRVPEEALKHEKFTIQLQLSQKSSESELSKSASAKSIDSKVADAATEVQHKTTEALKSEEKAMDISAMPRGTPLYGQPSWWGDDEVDEKRAFKTNGKPEEKNHEAGTSGCSIDAKQVEEQSAAANEEVLFPFCREPSYFEIPTKEFQQPSQITESTIHEIPTKDTPSSHITGAGHASFTIEFDDSTPGKVTIRDHVTKFTSDQRHKSKKSSPGTQDLLGIQTGMMAPENKVADWLAQNNPPQMLWERTEEDSKSIKSDVPVYLKRLKGNKHDDGTQSDSENAGAHRRCSKRATLEEHLRRHHSEHKKLQKVQATEKHQDQAVVFGVDDNQDYNRPVINEKHKDLIKDWALSSAAAVMEERKPLTTSGFHHSEEGTSSSGSKRWVSQWASLAANHTRHDQEERIMEFSAPLPLENETEISESGMTVRSTGSATSLASQGERRRRTLPQLPNEEKSLESHRAKVVTQRSEIGEKQDTELQEKETPTQVYQKDKQDADRPLSKMNRAVNGETLKTGGDNKTLLHLGSSAPGKEKSETDKETSLVKQTLAKLQQQEQREEAQWTPTKLSSKNVSGQTDKCREETFKQESQPPEKNSGHSTSKGDRVAQSESKRRKAEEILKSQTPKGGDKKESSKSLVRQGSFTIEKPSPNIPIELIPHINKQTSSTPSSLALTSASRIRERSESLDPDSSMDTTLILKDTEAVMAFLEAKLREDNKTDEGPDTPSYNRDNSISPESDVDTASTISLVTGETERKSTQKRKSFTSLYKDRCSTGSPSKDVTKSSSSGAREKMEKKTKSRSTDVGSRADGRKFVQSSGRIRQPSVDLTDDDQTSSVPHSAISDIMSSDQETYSCKPHGRTPLTSADEHVHSKLEGSKVTKSKTSPVVSGSSSKSTTLPRPRPTRTSLLRRARLGEASDSELADADKASVASEVSTTSSTSKPPTGRRNISRIDLLAQPRRTRLGSLSARSDSEATISRSSASSRTAEAIIRSGARLVPSDKFSPRIRANSISRLSDSKVKSMTSAHGSASALKTTRLQSAGSAMPTSSSFKHRIKEQEDYIRDWTAHREEIARISQDLALIAREINDVAGEIDSVTSSGTAPSTTVSTAATTPGSAIDTREELVDRVFDESLNFQKIPPLVHSKTPEGNNGRSGDPRPQAAEPPDHLTITRRRTWSRDEVMGDNLLLSSVFQFSKKIRQSIDKTAGKIRILFKDKDRNWDDIESKLRAESEVPIVKTSSMEISSILQELKRVEKQLQAINAMIDPDGTLEALNNMGFPSAMLPSPPKQKSSPVNNHHSPGQTPTLGQPEARALHPAAVSAAAEFENAESEADFSIHFNRVNPDGEEEDVTVHK

SEQ ID NO: 8 - protein, 647 aa, Homo sapiens
  misc_feature: Incyte ID No 7504326CD1
MVMYARKQQRLSDGCHDRRGDSQPYQALKYSSKSHPSSGDHRHEKMRDAGDPSPPNKMLRRSDSPENKYSDSTGHSKAKNVHTHRVRERDGGTSYSPQENSHNHSALHSSNSHSSNPSNNPSKTSDAPYDSADDWSEHISSSGKKYYYNCRTEVSQWEKPKEWLEREQRQKEANKMAVNSFPKDRDYRREVMQATATSGFASGMEDKHSSDASSLLPQNILSQTSRHNDRDYRLPRAETHSSSTPVQHPIKPVVHPTATPSTVPSSPFTLQSDHQPKKSFDANGASTLSKLPTPTSSVPAQKTERKESTSGDKPVSHSCTTPSTSSASGLNPTSAPPTSASAVPVSPVPQSPIPPLLQDPNLLRQLLPALQATLQLNNSNVDISKINEVLTAAVTQASLQSIIHKFLTAGPSAFNITSLISQAAQLSTQAQPSNQSPMSLTSDASSPRSYVSPRISTPQTNTVPIKPLISTPPVSSQPKVSTPVVKQGPVSQSATQQPVTADKQQGHEPVSPRSLQRSSSQRSPSPGPNHTSNSSNASNATVVPQNSSARSTCSLTPALAAHFSENLIKHVQGWPADHAEKQASRLREEAHNMGTIHMSEICTELKNLRSLVRVCEIQATLREQRILFLRQQIKELEKLKNQNSFMV

SEQ ID NO: 9 - protein, 195 aa, Homo sapiens
  misc_feature: Incyte ID No 7504388CD1
MAPPAAPGRDRVGREDEDGWETRGDRKVQAKLENAEVLELTVRRVQGVLRGRAREREQLQAEASERFAAGYIQCMHEVHTFVSTCQAIDATVAAELLNHLLESMPLREGSSFQDLLGDALAGPPRAPGRSGWPAGGAPGSPIPSPPGPGDDLCSDLEEAPEAELSQAPAEGPDLVPAALGSLTTAQIARSVWRPW

SEQ ID NO: 10 - protein, 781 aa, Homo sapiens
  misc_feature: Incyte ID No 2828380CD1
MATQGHLTFKDVAIEFSQEEWKCLEPVQKALYKDVMLENYRNLVFLGISPKCVIKELPPTENSNTGERFQTVALERHQSYDIENLYFREIQKHLHDLEFQWKDGETNDKEVPVPHENNLTGKRDQHSQGDVENNHIENQLTSNFESRLAELQKVQTEGRLYECNETEKTGNNGCLVSPHIREKTYVCNECGKAFKASSSLINHQRIHTTEKPYKCNECGKAFHRASLLTVHKVVHTRGKSYQCDVCGKIFRKNSYFVRHQRSHTGQKPYICNECGKSFSKSSHLAVHQRIHTGEKPYKCNLCGKSFSQRVHLRLHQTVHTGERPFKCNECGKTFKRSSNLTVHQVIHAGKKPYKCDVCGKAFRHRSNLVCHRRIHSGEKQYKCNECGKVFSKRSSLAVHRRIHTVEKPCKCNECGKVFSKRSSLAVHQRIHTGQKTYKCNKCGKVYSKHSHLAVHWRIHTGEKAYKCNECGKVFSIHSRLAAHQRIHTGEKPYKCNECGKVFSQHSRLAVHRRIHTGEKPYKCKECGKVFSDRSAFARHRRIHTGEKPYKCKECGKVFSQCSRLTVHLRIHSGEKPYKCNECGKVYSQYSHLVGHRRVHTGEKPYKCHECGKAFNQGSTLNRHQRIHTGEKPYKCNQCGNSFSQRVHLRLHQTVHTGDRPYKCNECGKTFKRSSNLTAHQIIHAGKKPYKCDECGKVFRHSSHLVSHQRIHTGEKRYKCIECGKAFGRLFSLSKHQRIHSGKKPYKCNECGKSFICRSGLTKHRIRHTGESLTTKLNVTRP

SEQ ID NO: 11 - protein, 595 aa, Homo sapiens
  misc_feature: Incyte ID No 6456919CD1
MDPVAFKDVAVNFTQEEWALLDISQRKLYREVMLETFRNLTSLGKRWKDQNIEYEHQNPRRNFRSLIEEKVNEIKDDSHCGETFTPVPDDRLNFQEKKASPEVKSCESFVCGEVGLGNSSFNMSIRGDIGHKAYEYQEYGPKPCKCQQPKKAFRYRPSFRTQERDHTGEKPNACKVCGKTFISHSSVRRHMVMHSGDGPYKCKFCGKAFHCLRLYLIHERIHTGEKPCECKQCGKSFSYSATHRIHKRTHTGEKPYEYQECGKAFHSPRSYRRHERIHMGEKAYQCKECGKAFTCPRYVRIHERTHSRKNLYECKQCGKALSSLTSFQTHVRLHSGERPYECKICGKDFCSVNSFQRHEKIHSGEKPYKCKQCGKAFPHSSSLRYHERTHTGEKPYECKQCGKAFRSASHLRVHGRTHTGEKPYECKECGKAFRYVNNLQSHERTQTHIRIHSGERRYKCKICGKGFYCPKSFQRHEKTHTGEKLYECKQRSVVPSVVPVPFDIMKGLTLERSPINASNVGKPSELCQSFECMVGLTLKRNPMSVSNDGKPSDLPHTFEYVVGHTMERNPMHVRNVGNPSDLPRTFEFMKGHKHT

SEQ ID NO: 12 - protein, 226 aa, Homo sapiens
  misc_feature: Incyte ID No 7502244CD1
MDQARGLDDAAARGGQCPGLGPAPTPTPPGRLGAPYSEAWGYFHLAPGRPGHPSGHWATCRLCGEQVGRGPGFHAGTSALWRHLRSAHRRELESSGAGSSPPAAPCPPPPVPAACPEGDWARLLEQMGALAVRGSLAGAGAGSGAEAAVEQGERALERRRRALQEEERAAAQARRELQAEREALQARLRDVSRREGALGWAPAAPPPLKDDPEGDRDGCVITKVLL

SEQ ID NO: 13 - protein, 548 aa, Homo sapiens
  misc_feature: Incyte ID No 7498718CD1
MFPVFSGCFQELQEKNKSLELVSFEEVAVHFTWEEWQDLDDAQRTLYRDVMLETYSSLVSLGHCITKPEMIFKLEQGAEPWIVEETLNLRLSAVQIIDDLIERSHESHDRFFWQIVITNSNTSTQERVELGKTFNLNSNHVLNLIINNGNSSGMKPGQFNDCQNMLFPIKPGETQSGEKPHVCDITRRSHRHHEHLTQHHKIQTLVQTFQCNEQGKTFNTEAMFFIHKRVHIVQTFGKYNEYEKACNNSAVIVQGITQVGQPTCCRKSDFTKHQQTHTGEKPYECVECEKPSISKSDLMLQCKMPTEEKPYACNWCEKLFSYKSSLIIHQRIHTGEKPYGCNECGKTFRCKSFLTLHERTHTGDKPYKCIECGKTFHCKSLLTLHHRTHSGEKPYQCSECGKTFSQKSYLTIHHRTHTGEKPYACDHCEEAFSHKSRLTVHQRTHTGEKPYECNECGKPFINKSNLRLHQRTHTGEKPYECNECGKTFHRKSFLTIHQWTHTGEKPYECNECGKTFRCKSFLTVHQRTHAGEKPYACNECGKTYSHKS

SEQ ID NO: 14 - protein, 264 aa, Homo sapiens
  misc_feature: Incyte ID No 6259308CD1
MPDSAPAMADKMDMSLDDIIKLNRSQRGGRGGGRGRGRAGSQGGRGGGAQAAARVNRGGGPIRNRPAIARGAAGGGGRNRPAPYSRPKQLPDKWQHDLFDSGFGGGAGVETGGKLLVSNLDFGVSDADIQELFAEFGTLKKAAVHYDRSGRSLGTADVHFERKADALKAMKQYNGVPLDGRPMNIQLVTSQIDAQRRPAQSVNRGGMTRNRGAGGFGGGGGTRRGTRGGARGRGRGAGRNSKQQLSAEELDAQLDAYNARMDTS

SEQ ID NO: 15 - protein, 611 aa, Homo sapiens
  misc_feature: Incyte ID No 7504104CD1
MHHQQRMAALGTDKELSDLLDFSAMFSPPVSSGKNGPTSLASGHFTGSNVEDRSSSGSWGNGGHPSPSRNYGDGTPYDHMTSRDLGSHDNLSPPFVNSRIQSKTERGSYSSYGRESNLQGCHQVYAPSASTADYNRDSPGYPSSKPATSTFPSSFFMQDGHHSSDPWSSSSGMNQPGYAGMLGNSSHIPQSSSYCSLHPHERLSYPSHSSADINSSLPPMSTFHRSGTNHYSTSSCTPPANGTDSIMANRGSGAAGSSQTGDALGKALASIYSPDHTNNSFSSNPSTPVGSPPSLSAGTAVWSRNGGQASSSPNYEGPLHSLQSRIEDRLERLDDAIHVLRNHAVGPSTAMPGGHGDMHGIIGPSHNGAMGGLGSGYGTGLLSANRHSLMVGTHREDGVALRGSHSLLPNQVPVPQLPVQSATSPDLNPPQDPYRGMPPGLQGQSVSSGSSEIKSDDEGDENLQDTKSSEDKKLDDDKKDIKSITRSRSSNNDDEDLTPEQKAEREKERRMANNARERLRVRDINEAFKELGRMVQLHLKSDKPQTKLLILHQAVAVILSLEQQVRERNLNPKAACLKRREEEKVSSEPPPLSLAGPHPGMGDASNHMGQM

SEQ ID NO: 16 - protein, 386 aa, Homo sapiens
  misc_feature: Incyte ID No 7504121CD1
MPQLSGGGGGGGGDPELCATDEMIPFKDEGDPQKEKIFAEISHPEEEGDLADIKSSLVNESEIIPASNGHEVARQAQTSQEPYHDKAREHPDDGKHPDGGLYNKGPSYSSYSGYIMMPNMNNDPYMSNGSLSPPIPRTSNKVPVVQPSHAVHPLTPLITYSDEHFSPGSHPSHIPSDVNSKQGMSRHPPAPDIPTFYPLSPGGVGQITPPLGWFSHHMIPGPPGPHTTGIPHPAIVTPQVKQEHPHTDSDLMHVKPQHEQRKEQEPKRPHIKKPLNAFMLYMKEMRANVVAECTLKESAAINQILGRRWHALSREEQAKYYELARKERQLHMQLYPGWSARDNYGKKKKRKREKLQESASGGKRSSFPTCKAKAATPGPLLEMEAC

SEQ ID NO: 17 - protein, 807 aa, Homo sapiens
  misc_feature: Incyte ID No 5635695CD1
MSKLSFRARALDAAKPLPIYRGKDMPDLNDCVSINRAVPQMPTGMEKEEESEHHLQRAISAQQVFREKKESMVIPVPEAESNVNYYNRLYKGEFKQPKQFIHIQPFNLDNEQPDYDMDSEDETLLNRLNRKMEIKPLQFEIMIDRLEKASSNQLVTLQEAKLLLNEDDYLIKAVYDYWVRKRKNCRGPSLIPQIKQEKRDGSTNNDPYVAFRRRTEKMQTRKNRKNDEASYEKMLKLRREFSRAITILEMIKRREKTKRELLHLTLEVVEKRYHLGDYGGEILNEVKISRSEKELYATPATLHNGNHHKVQECKTKHPHHLSLKEEASDVVRQKKKYPKKPKAEALITSQQPTPETLPVINKSDIKQYDFHSSDEDEFPQVLSPVSEPEEENDPDGPCAFRRRAGCQYYAPRLDQANHSCENSELADLDKLRYRHCLTTLTVPRRCIGFARRRIGRGGRVIMDRISTEHDPVLKQIDPEMLNSFSSSSQTIDFSSNFSRTNASSKHCENRLSLSEILSNIRSCRLQCFQPRLLNLQDSDSEECTSRKPGQTVNNKRVSAASVALLNTSKNGISVTGGITEEQFQTHQQQLVQMQRQQLAQLQQKQQSQHSSQQTHPKAQGSSTSDCMSKTLDSASAHFAASAVVSAPVPSRSEVAKEQNTGHNNINGVVQPSGTSKTLYSTNMALSSSPGISAVQLVRTVGHTTTNHLIPALCTSSPQTLPMNNSCLTNAVHLNNVSVVSPVNVHINTRTSAPSPTALKLATVAASMDRVPKVTPSSAISSIARENHEPERLGLNGIAETTVAMEVT

SEQ ID NO: 18 - protein, 257 aa, Homo sapiens
  misc_feature: Incyte ID No 7503983CD1
MATNFLAHEKIWFDKFKYDDAERRFYEQMNGPVAGASRQSSGPGASSGTSGDHGELVVRIASLEVENQSLRGVVQELQQAISKLEARLNVLEKSSPGHRATAPQTQHVSPMRQVEPPAKKPATPAEDDEDDDIDLFGSDNEEEDKEAAQLREERLRQYAEKKAKKPALVAKSSILLDVKPWDDETDMAQLEACVRSIQLDGLVWGASKLVPVGYGIRKLQIQCVVEDDKVGTDLLEEEITKFEEHVQSVDIAAFNKI

SEQ ID NO: 19 - protein, 113 aa, Homo sapiens
  misc_feature: Incyte ID No 7503476CD1
MPSRLRKTRKLRGHVSHGHGRIGKHRKHPGGRGNAGGLHHHRINFDKYEQTRVNAAKNKTGAAPIIDVVRSGYYKVLGKGKLPKQPVIVKAKFFSRRAEEKIKSVGGACVLVA

SEQ ID NO: 20 - protein, 204 aa, Homo sapiens
  misc_feature: Incyte ID No 7504023CD1
MPREDRATWKSNYFLKIIQLLDDYPKCFIVGADNVGFVFTKEDLTEIRDMLLANKVPAAARAGAIAPCEVTVPAQNTGLGPEKTSFFQALGITTKISRGTIEILGVRNVASVCLQIGYPTVASVPHSIINGYKRVLALSVETDYTFPLAEKVKAFLADPSAFVAAAPVAAATTAAPAAAAAPAKVEAKEESEESDEDMGFGLFD

SEQ ID NO: 21 - protein, 144 aa, Homo sapiens
  misc_feature: Incyte ID No 7504128CD1
MPPKFDPNEIKVVYLRCTGGEVGATSALAPKIGPLRITVKLTIQNRQAQIEVVPSASALIIKALKEPPRDRKKQKNIKHSGNITFDEIVNIARQMRHRSLARELSGTIKEILGTAQSVGCNVDGRHPHDIIDDINSGAVECPAS

SEQ ID NO: 22 - protein, 355 aa, Homo sapiens
  misc_feature: Incyte ID No 4529338CD1
MLSLSSLRRNSGRNSGSCGAWNMVGEMETKEKPKPTPDYLMQLMNDKKLMSSLPNFCGIFNHLERLLDEEISRVRKDMYNDTLNGSTEKRSAELPDAVGPIVQLQEKLYVPVKEYPDFNFVGRILGPRGLTAKQLEAETGCKIMVRGKGSMRDKKKEEQNRGKPNWEHLNEDLHVLITVEDAQNRAEIKLKRAVEEVKKLLVPAAEGEDSLKKMQLMELAILNGTYRDANIKSPTAQAAPRIITGPAPVLPPAALRTPTPAGPTIMPLIRQIQTAVMPNGTPHPTAAIVPPGPEAGLIYTPYEYPYTLAPATSILEYPIEPSGVLGAVATKVRRHDMRVHPYQRIVTADRAATGN

SEQ ID NO: 23 - protein, 143 aa, Homo sapiens
  misc_feature: Incyte ID No 7503460CD1
MPAGPVQAVPPPPPVPTEPKQPTEEEASSKEDSAPSKPVVGIIYPPPEVRNIVDKTASFVARNGPEFEARIRQNEINNPKFNFLNPNDPYHAYYRHKVSEFKEGKAQEPSAAIPKVMQQQQQTTQQQLPSYFLCIRIIFVLPF

SEQ ID NO: 24 - protein, 1048 aa, Homo sapiens
  misc_feature: Incyte ID No 5466630CD1
MRLFYTTSKLGTGNRMYHTKEKADEVVAPGQEKISSLSGAHRKRRRWPQLRRRTEEEEESESELEEESELDEDPAAEPAEAGVGTTVAPLPPAPAPSSQPVPAGMTVPPPPAAAPPLPRALAKPAVFIPVNRSPEMQEERLKLPILSEEQVIMEAVAEHPIVIVCGETGSGKTTQVPQFLYEAGFSSEDSIIGVTEPRRVAAVAMSQRVAKEMNLSQRVVSYQIRYEGNVTEETRIKFMTDGVLLKEIQKDFLLLRYKVVIIDEAHERSVYTDILIGLLSRIVTLRAKRNLPLKLLIMSATLRVEDFTQNPRLFAKPPPVIKVESRQFPVTVHFNKRTPLEDYSGECFRKVCKIHRMLPAGGILVFLTGQAEVHALCRRLRKAFPPSRARPQEKDDDQKDSVEEMRKFKKSRARAKKARAEVLPQINLDHYSVLPAGEGDEDREAEVDEEEGALDSDLDLDLGDGGQDGGEQPDASLPLHVLPLYSLLAPEKQAQVFKPPPEGTRLCVVATNVAETSLTIPGIKYVVDCGKVKKRYYDRVTGVSSFRVTWVSQASADQRAGRAGRTEPGHCYRLYSSAVFGDFEQFPPPEITRRPVEDLILQMKALNVEKVINFPFPTPPSVEALLAAEELLIALGALQPPQKAERVKQLQENRLSCPITALGRTMATFPVAPRYAKMLALSRQHGCLPYAITIVASMTVRELFEELDRPAASDEELTRLKSKRARVAQMKRTWAGQGASLKLGDLMVLLGAVGACEYAGCTPQFCEANGLRYKAMMEIRRLRGQLTTAVNAVCPEAELFVDPKMQPPTESQVTYLRQIVTAGLGDHLARRVQSEEMLEDKWRNAYKTPLLDDPVFIHPSSVLFKELPEFVVYQEIVETTKMYMKGVSSVEVQWIPALLPSYCQFDKPLEEPAPTYCPERGRVLCHRASVFYRVGWPLPAIEVDFPEGIDRYKHFARFLLEGQVFRKLASYQSCLLSSPGTMLKTWARLQPRTESLLRALVAEKADCHEALLAAWKKNPKYLLAEYCEWLPQAMHPDIEKAWPPTTVH

SEQ ID NO: 25 - protein, 294 aa, Homo sapiens
  misc_feature: Incyte ID No 7503474CD1
MSVNYAAGLSPYADKGKCGLPEIFDPPEELERKVWELARLVWQSSNVVFHTGAGISTASGIPDFRDKLAELHGNMFVEECAKCKTQYVRDTVVGTMGLKATGRLCTVAKARGLRACRGELRDTILDWEDSLPDRDLALADEASRNADLSITLGTSLQIRPSGNLPLATKRRGGRLVIVNLQPTKHDRHADLRIHGYVDEVMTRLMKHLGLEIPAWDGPRVLERALPPLPRPPTPKLEPKEESPTRINGSIPAGPKQEPCAQHNGSEPASPKRERPTSPAPHRPPKRVKAKAVPS

SEQ ID NO: 26 - protein, 280 aa, Homo sapiens
  misc_feature: Incyte ID No 7503498CD1
MNTGPYYFVKNLPLHELITPEFISTFIKKGKLILSLDKDTYEETGLQGHPSQFSGRKIMKFIVSIDLMELSLNLDSKKYERISWSFKEKKPLKFDFLLAWHKTGSEESTMMSYFSKYQIQEHQPKVALSTLRDLQCPVLQSSELEGTPEVSCRALELFDWLGAVFSNVDLNNEPNNFISTYCCPEPSTVVAKAYLCTITGFILPEKICLLLEHLCHYFDEPKLAPWVTLSVQGFADSPVSWEKNEHGFRKGGEHLYNFVIFNNQDYWLQMAVGANDHCPP

SEQ ID NO: 27 - protein, 288 aa, Homo sapiens
  misc_feature: Incyte ID No 7504119CD1
MAASGKLSTCRLPPLPTIREIIKLLRLQAAKQLSQNFLLDLRLTDKIVRKAGNLTNAYVYEVGPGPGGITRSILNADVAELLVVEKDTRFIPGLQMLSDAAPGKLRIVHGDVLTFKVEKAFSESLKRPWEDDPPNVHIIGNLPFSVSTPLIIKWLENISCRDGPFVYGRTQMTLTFQKEVAERLAANTGSKQRSRLSVMAQYLCNVRHIFTIPGQAFVPKPEVDVGVVHFTPLIQPKIEQPFKLVEKVVQNVFQFRRKYCHRGLREELKRRKSKNEEKEEDDAENYRL

SEQ ID NO: 28 - protein, 244 aa, Homo sapiens
  misc_feature: Incyte ID No 71532805CD1
MSSVASKVAVPESVLRKRKREEQWATEKKEKALVEKKKSIESRKLIFTRAKQYAEEYDAQEKELVQLKREARLKGGFYVSPEAKLLFVVRIRGINAMHPKTRKILQLLRLRQIFNGVFLKVNKATINMLRRVEPYVAYGYPNLKSVRELIYKRGYGKLNKQRIPLSNNQVIEEGLGKHNIICIEDLVHEIMTVGPHFKEANNFLWPFKLKAPLGGLKKKRNHYVEGGDAGNRENYINELIKRMN

SEQ ID NO: 29 - protein, 1953 aa, Homo sapiens
  misc_feature: Incyte ID No 5502992CD1
MQFFVENPSEEDAAIVDKVLSMRIVKKELPSGPYTEAEEFFVKYKNYSYLHCEWATISQLEKDKRIHQKLKRFKTKMAQMRHFFHEDEEPFNPDYVEVDRILDESHSIDKDNGEPVIYYLVKWCSLPYEDSTWELKEDVDEGKIREFKRIQSRHPELKRVNRPQASAWKKLELSHEYKNRNQLREYQLEGVNWLLFNWYNRQNCILADEMGLGKTIQSIAFLQEVYNVGIHGPFLVIAPLSTITNWEREFNTWTEMNTIVYHGSLASRQMIQQYEMYCKDSRGRLIPGAYKFDALITTFEMILSDCPELREIEWRCVIIDEAHRLKNRNCKLLDSLKHMDLEHKVLLTGTPLQNTVEELFSLLHFLEPSQFPSESEFLKDFGDLKTEEQVQKLQAILKPMMLRRLKEDVEKNLAPKQETIIEVELTNIQKKYYRAILEKNFSFLSKGAGHTNMPNLLNTMMELRKCCNHPYLINGAEEKILTEFREACHIIPHDFHLQAMVRSAGKLVLIDKLLPKLKAGGHKVLIFSQMVRCLDILEDYLIQRRYLYERIDGRVRGNLRQAAIDRFSKPDSDRFVFLLCTRAGGLGINLTAADTCIIFDSDWNPQNDLQAQARCHRIGQSKAVKVYRLITRNSYEREMFDKASLKLGLDKAVLQSMSGRDGNITGIQQFSKKEIEDLLRKGAYAAIMEEDDEGSKFCEEDIDQILLRRTTTITIESEGKGSTFAKASFVASENRTDISLDDPNFWQKWAKKADLDMDLLNSKNNLVIDTPRVRKQTRHFSTLKDDDLVEFSDLESEDDERPRSRRHDRHHAYGRTDCFRVEKHLLVYGWGRWRDILSHGRFKRRMTERDVETICRAILVYCLLHYRGDENIKGFIWDLISPAENGKTKELQNHSGLSIPVPRGRKGKKVKSQSTFDIHKADWIRKYNPDTLFQDESYKKHLKHQCNKVLLRVRMLYYLRQEVIGDQAEKVLGGAIASEIDIWFPVVDQLEVPTTWWDSEADKSLLIGVFKHGYEKYNTMRADPALCFLEKAGRPDDKAIAAEHRVLDNFSDIVEGVDFDKDCEDPEYKPLQGPPKDQDDEGDPLMMMDEEISVIDGDEAQVTQQPGHLFWPPGSALTARLRRLVTAYQRSYKREQMKIEAAERGDRRRRRCEAAFKLKEIARREKQQRWTRREQTDFYRVVSTFGVEYDPDTMQFHWDRFRTFARLDKKTDESLTKYFHGFVAMCRQVCRLPPAAGDEPPDPNLFIEPITEERASRTLYRIELLRRLREQVLCHPLLEDRLALCQPPGPELPKWWEPVRHDGELLRGAARHGVSQTDCNIMQDPDFSFLAARMNYMQNHQAGAPAPSLSRCSTPLLHQQYTSRTASPLPLRPDAPVEKSPEETATQVPSLESLTLKLEHEVVARSRPTPQDYEMRVSPSDTTPLVSRSVPPVKLEDEDDSDSELDLSKLSPSSSSSSSSSSSSSSTDESEDEKEEKLTDQSRSKLYDEESLLSLTMSQDGFPNEDGEQMTPELLLLQERQRASEWPKDRVLINRIDLVCQAVLSGKWPSSRRSQEMVTGGILGPGNHLLDSPSLTPGEYGDSPVPTPRSSSAASMAEEEASAVSTAAAQFTKLRRGMDEKEFTVQIKDEEGLKLTFQKHKLMANGVMGDGHPLFHKKKGNRKKLVELEVECMEEPNHLDVDLETRIPVINKVDGTLLVGEDAPRRAELEMWLQGHPEFAVDPRFLAYMEDRRKQKWQRCKKNNKAELNCLGMEPVQTANSRNGKKGHHTETVFNRVLPGPIAPESSKKRARRMRPDLSKMMALMQGGSTGSLSLHNTFQHSSSGLQSVSSLGHSSATSASLPFMPFVMGGAPSSPHVDSSTMLHHHHHHPHPHHHHHHHPGLRAPGYPSSPVTTASGTTLRLPPLQPEEDDDEDEEDDDDLSQGYDSSERDFSLIDDPMMPANSDSSEDADD

SEQ ID NO: 30 - protein, 1099 aa, Homo sapiens
  misc_feature: Incyte ID No 7503828CD1
MAVRKKDGGPNVKYYEAADTVTQFDNVRLWLGKNYKKYIQAEPPTNKSLSSLVVQLLQFQEEVFGKHVSNAPLTKLPIKCFLDFKAGGSLCHILAAAYKFKSDQGWRRYDFQNPSRMDRNVEMFMTIEKSLVQNNCLSRPNIFLCPEIEPKLLGKLKDIIKRHQGTVTEDKNNASHVVYPVPGNLEEEEWVRPVMKRDKQVLLHWGYYPDSYDTWIPASEIEASVEDAPTPEKPRKVHAKWILDTDTFNEWMNEEDYEVNDDKNPVSRRKKISAKTLTDEVNSPDSDRRDKKGGNYKKRKRSPSPSPTPEAKKKNAKKGPSTPYTKSKRGHREEEQEDLTKDMDEPSPVPNVEEVTLPKTVNTKKDSESAPVKGGTMTDLDEQEDESMETTGKDEDENSTGNKGEQTKNPDLHEDNVTEQTHHIIIPSYAAWFDYNSVHAIERRALPEFFNGKNKSKTPEIYLAYRNFMIDTYRLNPQEYLTSTACRRNLAGDVCAIMRVHAFLEQWGLINYQVDAESRPTPMGPPPTSHFHVLADTPSGLVPLQPKTPQQTSASQQMLNFPDKGKEKPTDMQNFGLRTDMYTKKNVPSKSKAAASATREWTEQETLLLLEALEMYKDDWNKVSEHVGSRTQDECILHFLRLPIEDPYLEDSEASLGPLAYQPIPFSQSGNPVMSTVAFLASVVDPRVASAAAKSALEEFSKMKEEVPTALVEAHVRKVEEAAKVTGKADPAFGLESSGIAGTTSDEPERIEESGNDEARVEGQATDEKKEPKEPREGGGAIEEEAKEKTSEAPKKDEEKGKEGDSEKESEKSDGDPIVDPEKEKEPKEGQEEVLKEVVESEGERKTKVERDIGEGNLSTAAAAALAAAAVKAKHLAAVEERKIKSLVALLVETQMKKLEIKLRHFEELETIMDREREALEYQRQQLLADRQAFHMEQLKYAEMRARQQHFQQMHQQQQQPPPALPPGSQPIPPTGAAGPPAVHGLAVAPASVVPAPAGSGAPPGSLGPSEQIGQAGSTAGPQQQQPAGAPQPGAVPPGVPPPGPHGPSPFPNQQTPPSMMPGAVPGSGHPGVADPGTPLPPDPTAPSPGTVTPVPPPQ

SEQ ID NO: 31 - protein, 203 aa, Homo sapiens
  misc_feature: Incyte ID No 2647325CD1
MTELASSGGGSPAGDGEEGLGDERGLVIHHPAEEQPYRCPLCGQTFSQQPSLVRHQKAHAGAGRAAAFVCPECGKAFSVKHNLEVHQRTHTGERPFPCPECGRCFSLKQNLLTHQRIHSGEKPHQCAQCGRCFREPRFLLNHQRTHARMPAPHPRRPGVFGERRPYFCPRCGKSFAREGSLKTHQRSHGHGPEGQAAHLGRVL

SEQ ID NO: 32 - protein, 317 aa, Homo sapiens
  misc_feature: Incyte ID No 7495416CD1
MPGEQQAEEEEEEEMQEEMVLLVKGEEDEGEEKYEVVKLKIPMDNKEVPGEAPAPSADPARPHACPDCGRAFARRSTLAKHARTHTGERPFGCTECGRRFSQKSALTKHGRTHTGERPYECPECDKRFSAASNLRQHRRRHTGEKPYACAHCGRRFAQSSNYAQHLRVHTGEKPYACPDCGRAFGGSSCLARHRRTHTGERPYACADCGTRFAQSSALAKHRRVHTGEKPHRCAVCGRRFGHRSNLAEHARTHTGERPYPCAECGRRFRLSSHFIRHRRAHMRRRLYICAGCGRDFKLPPGATAATATERCPECEGS

SEQ ID NO: 33 - protein, 579 aa, Homo sapiens
  misc_feature: Incyte ID No 8096177CD1
MNKSQEQVSFKDVCVDFTQEEWYLLDPAQKILYRDVILENYSNLVSVGYCITKPEVIFKIEQGEEPWILEKGFPSQCHPERKWKVDDVLESSQENEDDHFWELLFHNNKTVSVENGDRGSKTFNLGTDPVSLRNYPYKICDSCEMNLKNISGLIISKKNCSRKKPDEFNVCEKLLLDIRHEKIPIGEKSYKYDQKRNAINYHQDLSQPSFGQSFEYSKNGQGFHDEAAFFTNKRSQIGETVCKYNECGRTFIESLKLNISQRPHLEMEPYGCSICGKSFCMNLRFGHQRALTKDNPYEYNEYGEIFCDNSAFIIHQGAYTRKILREYKVSDKTWEKSALLKHQIVHMGGKSYDYNENGSNFSKKSHLTQLRRAHTGEKTFECGECGKTFWEKSNLTQHQRTHTGEKPYECTECGKAFCQKPHLTNHQRTHTGEKPYECKQCGKTFCVKSNLTEHQRTHTGEKPYECNACGKSFCHRSALTVHQRTHTGEKPFICNECGKSFCVKSNLIVHQRTHTGEKPYKCNECGKTFCEKSALTKHQRTHTGEKPYECNACGKTFSQRSVLTKHQRIHTRVKALSTS

SEQ ID NO: 34 - protein, 730 aa, Homo sapiens
  misc_feature: Incyte ID No 666763CD1
MGSRSRNGLGNFNCGSTCWCGLGQRRRTNLVCKLNTRTTILAATNPKGQYDPQESVSVNIALGSPLLSRFDLILVLLDTKNEDWDRIISSFILENKGYPSKSEKLWSMEKMKTYFCLIRNLQPTLSDVGNQVLLRYYQMQRQSDSRNAARTTIRLLESLIRLAEAHARLMFRDTVTLEDAITVVSVMESSMQGGALLGGVNALHTSFPENPGEQYQRQCELILEKLELQSLLSEELRRLERLQNQSVHQSQPRVLEVETTPGSLRNGPGEESNFRTSSQQEINYSTHIFSPGGSPEGSPVLDPPPHLEPNRSTSRKHSAQHKNNRDDSLDWFDFMATHQSEPKNTVVVSPHPKTSGENMASKISNSTSQGKEKSEPGQRSKVDIGLLPSPGETGVPWRADNVESNKKKRLALDSEAAVSADKPDSVLTHHVPRNLQKLCKERAQKLCRNSTRVPAQCTVPSHPQSTPVHSPDRMLDSPKRKRPKSLAQVEEPAIENVKPPGSPVAKLAKFTFKQKSKLIHSFEDHSHVSPGATKIAVHSPKISQRRTRRDAALPVKRPGKLTSTPGNQISSQPQGETKEVSQQPPEKHGPREKVMCAPEKRIIQPELELGNETGCAHLTCEGDKKEEVSGSNKSGKVHACTLARLANFCFTPPSESKSKSPPPERKNRGERGPSSPPTTTAPMRVSKRKSFQLRGSTEKLIVSKESLFTLPELGDEAFDCDWDEEMRKKS

SEQ ID NO: 35 - protein, 315 aa, Homo sapiens
  misc_feature: Incyte ID No 7504091CD1
MPGWRLLTQVGAQVLGRLGDGLGAALGPGNRTHIWLFVRGLHGKSGTWWDEHLSEENVPFIKQLVSDEDKAQLASKLCPLKDEPWPIHPWEPGSFRVGLIALKLGMMPLWTKDGQKHVVTLLQKATSILEFYRELGLPPKQTVKIFNITDNAAIKPGTPLYAAHFRPGQYVDVTAKTIGKGFQGVMKRWGFKGQPATHGQTKTHRRPGAVATGDIGRVWPGTKMPGKMGNIYRTEYGLKVWRINTKHNIIYVNGSVPGHKNCLVKVKDSKLPAYKDLGKNLPFPTYFPDGDEEELPEDLYDENVCQPGAPSITFA

SEQ ID NO: 36 - protein, 317 aa, Homo sapiens
  misc_feature: Incyte ID No 7503568CD1
MDTGVIEGGLNVTLTIRLLMHGKEVGSIIGKKGESVKKMREESGARINISEGNCPERIITLAGPTNAIFKAFAMIIDKLEEDISSSMTNSTAASRPPVTLRLVVPASQCGSLIGKGGCKIKEIRESTGAQVQVAGDMLPNSTERAITIAGIPQSIIECVKQICVVMLESPPKGVTIPYRPKPSSSPVIFAGGQLTKLHQLAMQQSHFPMTHGNTGFSGIESSSPEVKGYWAGLDASAQTTSHELTIPNDLIGCIIGRQGAKINEIRQMSGAQIKIANPVEGSTDRQVTITGSAASISLAQYLINVRLSSETGGMGSS

SEQ ID NO: 37 - protein, 748 aa, Homo sapiens
  misc_feature: Incyte ID No 7504101CD1
MLEELECGAPGARGAATAMDCKDRPAFPVKKLIQARLPFKRLNLVPKGKADDMSDDQGTSVQSKSPDLEASLDTLENNCHVGSDIDFRPKLVNGKGPLDNFLRNRIETSIGQSTVIIDLTEDSNEQPDSLVDHNKLNSEASPSREAINGQREDTGDQQGLLKAIQNDKLAFPGETLSDIPCKTEEEGVGCGGAGRRGDSQECSPRSCPELTSGPRMCPRKEQDSWSEAGGILFKGKVPMVVLQDILAVRPPQIKSLPATPQGKNMTPESEVLESFPEEDSVLSHSSLSSPSSTSSPEGPPAPPKQHSSTSPFPTSTPLRRITKKFVKGSTEKNKLRLQRDQERLGKQLKLRAEREEKEKLKEEAKRAKEEAKKKKEEEKELKEKERREKREKDEKEKAEKQRLKEERRKERQEALEAKLEEKRKKEEEKRLREEEKRIKAEKAEITRFFQKPKTPQAPKTLAGSCGKFAPFEIKEHMVLAPRRRTAFHPDLCSQLDQLLQQQSGEFSFLKDLKGRQPLRSGPTHVSTRNADIFNSDVVIVERGKGDGVPERRKFGRMKLLQFCENHRPAYWGTWNKKTALIRARDPWAQDTKLLDYEVDSDEEWEEEEPGESLSHSEGDDDDDMGEDEDEDDGFFVPHGYLSEDEGVTEECADPENHKVRQKLKAKEWDEFLAKGKRFRVLQPVKIGCVWAADRDCAGDDLKVLQQFAACFLETLPAQEEQILEPTQFLCKEHFVLLHGPPQSVQSSI

SEQ ID NO: 38 - protein, 609 aa, Homo sapiens
  misc_feature: Incyte ID No 6946680CD1
MAVGLCKAMSQGLVTFRDVALDFSQEEWEWLKPSQKDLYRDVMLENYRNLVWLGLSISKPNMISLLEQGKEPWMVERKMSQGHCADWESWCEIEELSPKWFIDEDEISQEMVMERLASHGLECSSFREAWKYKGEFELHQGNAERHFMQVTAVKEISTGKRDNEFSNSGRSIPLKSVFLTQQKVPTIQQVHKFDIYDKLFPQNSVIIEYKRLHAEKESLIGNECEEFNQSTYLSKDIGIPPGEKPYESHDFSKLLSFHSLFTQHQTTHFGKLPHGYDECGDAFSCYSFFTQPQRIHSGEKPYACNDCGKAFSHDFFLSEHQRTHIGEKPYECKECNKAFRQSAHLAQHQRIHTGEKPFACNECGKAFSRYAFLVEHQRIHTGEKPYECKECNKAFRQSAHLNQHQRIHTGEKPYECNQCGKAFSRRIALTLHQRIHTGEKPFKCSECGKTFGYRSHLNQHQRIHTGEKPYECIKCGKFFRTDSQLNRHHRIHTGERPFECSKCGKAFSDALVLIHHKRSHAGEKPYECNKCGKAFSCGSYLNQHQRIHTGEKPYECSECGKAFHQILSLRLHQRIHAGEKPYKCNECGNNFSCVSALRRHQRIHNRETL

SEQ ID NO: 39 - protein, 536 aa, Homo sapiens
  misc_feature: Incyte ID No 7001142CD1
MAVGLLKAMYQELVTFRDVAVDFSQEEWDCLDSSQRHLYSNVMLENYRILVSLGLCFSKPSVILLLEQGKAPWMVKRELTKGLCSGWEPICETEELTPKQDFYEEHQSQKIIETLTSYNLEYSSLREEWKCEGYFERQPGNQKACFKEEIITHEEPLFDEREQEYKSWGSFHQNPLLCTQKIIPKEEKVHKHDTQKRSFKKNLMAIKPKSVCAEKKLLKCNDCEKVFSQSSSLTLHQRIHTGEKPYKCIECGKAFSQRSNLVQHQRIHTGEKPYECKECRKAFSQNAHLVQHLRVHTGEKPYECKVCRKAFSQFAYLAQHQRVHTGEKPYECIECGKAFSNRSSIAQHQRVHTGEKPYECNVCGKAFSLRAYLTVHQRIHTGERPYECKECGKAFSQNSHLAQHQRIHTGEKPYKCQECRKAFSQIAYLAQHQRVHTGEKPYECIECGKAFSNDSSLTQHQRVHTGEKPYECTVCGKAFSYCGSLAQHQRIHTGERPYECKECKKTFRQHAHLAHHQRIHIGESLSPPNPVNHQVL

SEQ ID NO: 40 - protein, 643 aa, Homo sapiens
  misc_feature: Incyte ID No 71158380CD1
MASVALEDVAVNFTREEWALLGPCQKNLYKDVMQETIRNLDCVGMKWKDQNIEDQYRYPRKNLRCRMLERFVESKDGTQCGETSSQIQDSIVTKNTLPGVGPYESRMSGEVIMGHSSLNCYIRVGAGHKPYEYHECGEKPDTHKQRGKAFSYHNSLQTHERLHTGKKPYNCKECGKSFSSLGNLQRHMAVQRGDGPYKCKLCGKAFFWPSLLHMHERTHTGEKPYECKQCSKAFSFYSSYLRHERTHTGEKLYECKQCSKAFPDYSSCLRHERTHTGKKPYTCKQCGKAFSASTSLRRHETTHTDEKPYACQQCGKAFHHLGSFQRHMVMHTRDGPHKCKICGKGFDCPSSLKSHERTHTGEKLYECKQCGKALSHSSSFRRHMTMHTGDGPHKCKICGKAFVYPSVFQRHEKTHTAEKPYKCKQCGKAYRISSSLRRHETTHTGEKPYKCKCGKAFIDFYSFQNHKTTHAGEKPYECKECGKAFSCFQYLSQHRRTHTGEKPYECNTCKKAFSHFGNLKVHERIHSGEKPYECKECGKAFSWLTCFLRHERIHMREKPYECQQCGKAFTHSRFLQGHEKTHTGENPYECKECGKAFASLSSLHRHKKTHWKKTHTGENPYGCKECGKAFASLSSLHRHKKTH

SEQ ID NO: 41 - protein, 1143 aa, Homo sapiens
  misc_feature: Incyte ID No 7503861CD1
MGDMKTPDFDDLLAAFDIPDPTSLDAKEAIQTPSEENESPLKPPGICMDESVSLSHSGSAPDVPAVSVIVKNTSRQESFEAEKDHITPSLLHNGFRGSDLPPDPHNCGKFDSTFMNGDSARSFPGKLEPPKSEPLPTFNQFSPISSPEPEDPIKDNGFGIKPKHSDSYFPPPLGCGAVGGPVLEALAKFPVPELHMFDHFCKKEPKPEPLPLGSQQEHEQSGQNTVEPHKDPDATRFFGEALEFNSHPSNSIGESKGLARELGTCSSVPPRQRLKPAHSKLSSCVAALVALQAKRVASVTKEDQPGHTKDLSGPTKESSKGSPKMPKSPKSPRSPLEATRKSIKPSDSPRSICSDSSSKGSPSVAASSPPAIPKVRIKTIKTSSGEIKRTVTRILPDPDDPSKSPVGSPLGSAIAEAPSEMPGDEVPVEEHFPEAGTNSGSPQGARKGDESMTKASDSSSPSCSSGPRVPKGAAPGSQTGKKQQSTALQASTLAPANLLPKAVHLANLNLVPHSVAASVTAKSSVQRRSQPQLTQMSVPLVHQVKKAAPLIVEVFNKVLHSSNPVPLYAPNLSPPADSRIHVPASGYCCLECGDAFALEKSLSQHYGRRSVHIEVLCTLCSKTLLPNQCSFCAHQRIHAHKSPYCCPECGVLCRSAYFQTHVKENCLHYARKVGYRCIHCGVVHLTLALLKSHIQERHCQVFHKCAFCPMAFKTASSTADHSATQHPTQPHRPSQLIYKCSCEMVFNKKRHIQQHFYQNVSKTQVGVFKCPECPLLFVQKPELMQHVKSTHGVPRNVDELSSLQSSADTSSSRPGSRVPTEPPATSVAARSSSLPSGRWGRPEAHRRVEARPRLRNTGWTCQECQEWVPDRESYVSHMKKSHGRTLKRYPCRQCEQSFHTPNSLRKHIRNNHDTVKKFYTCGYCTEDSPSFPRPSLLESHISLMHGIRNPDLSQTSKVKPPGGHSPQVNHLKRPVSGVGDAPGTSNGATVSSTKRHKSLFQCAKCSFATDSGLEFQSHIPQHQVDSSTAQCLLCGLCYTSASSLSRHLFIVHKVRDQEEEEEEEAAAAEMAVEVAEPEEGSGEEVPMETRENGLEECAGEPLSADPEARRLLGPAPEDDGGHNDHSQPQASQDQDSHTLSPQV

SEQ ID NO: 42 - protein, 1099 aa, Homo sapiens
  misc_feature: Incyte ID No 7758395CD1
MNLQAQPKAQNKRKRCLFGGQEPAPKEQPPPLQPPQQSIRVKEEQYLGHEGPGGAVSTSQPVELPPPSSLALLNSVVYGPERTSAAMLSQQVASVKWPNSVMAPGRGPERGGGGGVSDSSWQQQPGQPPPHSTWNCHSLSLYSATKGSPHPGVGVPTYYNHPEALKREKAGGPQLDRYVRPMMPQKVQLEVGRPQAPLNSFHAAKKPPNQSLPLQPFQLAFGHQVNRQVFRQGPPPPNPVAAFPPQKQQQQQQPQQQQQQQQAALPQMPLFENFYSMPQQPSQQPQDFGLQPAGPLGQSHLAHHSMAPYPFPPNPDMNPELRKALLQDSAPQPALPQVQIPFPRRSRRLSKEGILPPSALDGAGTQPGQEATGNLFLHHWPLQQPPPGSLGQPHPEALGFPLELRESQLLPDGERLAPNGREREAPAMGSEEGMRAVSTGDCGQVLRGGVIQSTRRRRRASQEANLLTLAQKAVELASLQNAKDGSGSEEKRKSVLASTTKCGVEFSEPSLATKRAREDSGMVPLIIPVSVPVRTVDPTEAAQAGGLDEDGKGPEQNPAEHKPSVIVTRRRSTRIPGTDAQAQAEDMNVKLEGEPSVRKPKQRPRPEPLIIPTKAGTFIAPPVYSNITPYQSHLRSPVRLADHPSERSFELPPYTPPPILSPVREGSGLYFNAIISTSTIPAPPPITPKSAHRTLLRTNSAEVTPPVLSVMGEATPVSIEPRINVGSRFQAEIPLMRDRALAAADPHKADLVWQPWEDLESSREKQRQVEDLLTAACSSIFPGAGTNQELALHCLHESRGDILETLNKLLLKKPLRPHNHPLATYHYTGSDQWKMAERKLFNKGIAIYKKDFFLVQKLIQTKTVAQCVEFYYTYKKQVKIGRNGTLTFGDVDTSDEKSAQEEVEVDIKTSQKFPRVPLPRRESPSEERLEPKREVKEPRKEGEEEVPEIQEKEEQEEGRERSRRAAAVKATQTLQANESASDILILRSHESNAPGSAGGQASEKPREGTGKSRRALPFSEKKKKTETFSKTQNQENTFPCKKCGRVFYKVKSRSAHMKSHAEQEKKAAALRLKEKEAAAAAAAAHQQALREESGAGDKG

SEQ ID NO: 43 - protein, 1006 aa, Homo sapiens
  misc_feature: Incyte ID No 71039312CD1
MDLGTAEGTRCTDPPAGKPAMAPKRKGGLKLNAICAKLSRQVVVEKRADAGSHTEGSPSQPRDQERSGPESGAARAPRSEEDKRRAVIEKWVNGEYSEEPAPTPVLGRIAREGLELPPEGVYMVQPQGCSDEEDHAEEPSKDGGALEEKDSDGAASKEDSGPSTRQASGEASSLRDYAASTMTEFLGMFGYDDQNTRDELARKISFEKLHAGSTPEAATSSMLPTSEDTLSKRARFSKYEEYIRKLKAGEQLSWPAPSTKTEERVGKEVVGTLPGLRLPSSTAHLETKATILPLPSHSSVQMQNLVARASKYDFFIQKLKTGENLRPQNGSTYKKPSKYDLENVKYLHLFKPGEGSPDMGGAIAFKTGKVGRPSKYDVRGIQKPGPAKVPPTPSLAPAPLASVPSAPSAPGPGPEPPASLSFNTPEYLKSTFSKTDSITTGTVSTVKNGLPTDKPAVTEDVNIYQKYIARFSGSQHCGHIHCAYQYREHYHCLDPECNYQRFTSKQDVIRHYNMHKKRDNSLQHGFMRFSPLDDCSVYYHGCHLNGKSTHYHCMQVGCNKVYTSTSDVMTHENFHKKNTQLINDGFQRFRATEDCGTADCQFYGQKTTHFHCRRPGCTFTFKNKCDIEKHKSYHIKDDAYAKDGFKKFYKYEECKYEGCVYSKATNHFHCIRAGCGFTFTSTSQMTSHKRKHERRHIRSSGALGLPPSLLGAKDTEHDESSNDDLVDFSALSSKNSSLSASPTSQQSSASLAAATAATEAGPSATKPPNSKISGLLPQGLPGSIPLALALSNSGLPTPTPYFPILAGRGSTSLPVGTPSLLGAVSSGSAASATPDTPTLVASGAGDSAPVAAASVPAPPASIMERISASKGLISPMMARLAAAALKPSATFDPGEQAGSCPESRHLLDWGGHLASGCRAESGGVWKKCPCGPCLPAPPLCLIFAPILLVSMWGPWFWLPTVGGPCFCFPICELGAMVPASSAALHPWGHCRSPLPVCSPSHIPSPP

SEQ ID NO: 44 - protein, 768 aa, Homo sapiens
  misc_feature: Incyte ID No 7291318CD1
MMLGLPSKEAALLFSGMDNQTVLAVQSLLDGQGAVPDPTGQSVNAPPAIQPLDDEDVFLCGKCKKQFNSLPAFMTHKREQCQGNAPALATVSLATNSIYPPSAAPTAVQQAPTPANRQISTYITVPPSPLIQTLVQGNILVSDDVLMSAMSAFTSLDQPMPQGPPPVQVPNQCVEPPVYPTPTVYSPGKQGFKPKGPNPAAPMTSATGGTVATFDSPATLKTRRAKAAGKPKAQKLKCSYCDKSFTKNFDLQQHIRSHTGEKPFQCIACGRAFAQKSNVKKHMQTHKVWPPGHSGGTVSRNSVTVQVMALNPSRQEDEESTGLGQPLPGAPQPQALSTAGEEEGDKPESKQVVLIDSSYLCQFCPSKFSTYFQLKSHMTQHKNEQVYKCVVKSCAQTFPKLDTFLEHIKSHQEELSYRCHLCGKDFPSLYDLGVHQYSHSLLPQHSPKKDNAVYKCVKCVNKYSTPEALEHHLQTATHNFPCPHCQKVFPCERYLRRHLPTHGSGGRFKCQVCKKFFRREHYLKLHAHIHSGEKPYKCSVCESAFNRKDKLKRHMLIHEPFKKYKCPFSTHTGCSKEFNRPDKLKAHILSQSGMKLHKCALCSKSFSRRAHLAEHQRAHTGNYKFRCAGCAKGFSRHKYLKDHRCRLGPQKDKDLQTRRPPQRRAAPRSCGSGGRKVLTPLPDPLGLEELKDTGAGLVPEAVPGKPPFAEPDAVLSIVVGGAVGAETELVVPGHAEGLGSNLALAELQAGAEGPCAMLAVPVYIQASE

SEQ ID NO: 45 - protein, 561 aa, Homo sapiens
  misc_feature: Incyte ID No 2638619CD1
MSSSRFWAGRANPASLPSQASSLGRQSPRVVSCLEHSLCPGEPGLQTTAVVSMGSGDHQFNLAEILSQNYSVRGECEEASRCPDKPKEELEKDFISQSNDMPFDELLALYGYEASDPISDRESEGGDVAPNLPDMTLDKEQIAKDLLSGEEEEETQSSADDLTPSVTSHEASDLFPNRSGSRFLADEDREPGSSASSDTEEDSLPANKCKKEIMVGPQFQADLSNLHLNRHCEKIYENEDQLLWDPASSLRGEVEEFLYRAVKRRWHEMAGPQLPEGEAVKDSEQALYELVKCNFNVEEALRRLRFNVKVIRDGLCAWSEEECRNFEHGFRVHGKNFHLIQANKVRTRSVGECVEYYYLWKKSERYDYFAQQTRLGRRKYVPSGTTDADQDLDGSDPDGPGRPRPEQDTLTGMRTDPLSVDGTAGGLDEPGVASDGLPSSEPGPCSFQQLDESPAVPLSHRPPALADPASYQPAVTAPEPDASPRLAVDFALPKELPLISSHVDLSGDPEETVAPAQVALSVTEFGLIGIGDVNPFLAAHPTCPAPGLHSEPLSHCNVMTC

SEQ ID NO: 46 - protein, 123 aa, Homo sapiens [sequence omitted]

SEQ ID NO: 47 - protein, 1236 aa, Homo sapiens
  misc_feature: Incyte ID No 3457155CD1
MSTAAFHISSLLEKMTSSDKDFRFMATSDLMSELQKDSIQLDEDSERKVVKMLLRLLEDKNGEVQNLAVKCLGPLVVKVKEYQVETIVDTLCTNMRSDKEQLRDIAGIGLKTVLSELPPAATGSGLATNVCRKITGQLTSAIAQQEDVAVQLEALDILSDMLSRLGVPLGAFHASLLHCLLPQLSSPRLAVRKRAVGALGHLAAACSTDLFVELADHLLDRLPGPRVPTSPTAIRTLIQCLGSVGRQAGHRLGAHLDRLVPLVEDFCNLDDDELRESCLQAFEAFLRKCPKEMGPHVPNVTSLCLQYIKHDPNYNYDSDEDEEQMETEDSEFSEQESEDEYSDDDDMSWKVRRAAAKCIAALISSRPDLLPDFHCTLAPVLIRRFKEREENVKADVFTAYIVLLRQTQPPKGWLEAMEEPTQTGSNLHMLRGQVPLVVKALQRQLKDRSVRARQGCFSLLTELAGVLPGSLAEHMPVLVSGIIFSLADRSSSSTIRMDALAFLQGLLGTEPAEAFHPHLPILLPPVMACVADSFYKIAAEALVVLQELVRALWPLHRPRMLDPEPYVGEMSAVTLARLRATDLDQEVKERAISCMGHLVGHLGDRLGDDLEPTLLLLLDRLRNEITRLPAIKALTLVAVSPLQLDLQPILAEALHILASFLRKNQRALRLATLAALDALAQSQGLSLPPSAVQAVLAELPALVNESDMHVAQLAVDFLATVTQAQPASLVEVSGPVLSELLRLLRSPLLPAGVLAAAEGFLQALVGTRPPCVDYAKLISLLTAPVYEQAVDGGPGLHKQVFHSLARCVAALSAACPQEAASTASRLVCDARSPHSSTGVKVLAFLSLAEVGQVAGPGPQRELKAVLLEALGSPSEDVRAAASYALGRVGAGSLPDFLPFLLEQIEAEPRRQYLLLHSLREALGAAQPDSLKPYAEDIWALLFQRCEGAEEGTRGVVAECIGKLVLVNPSFLLPRLRKQLAAGRPHTRSTVITAVKFLISDQPHPIDPLLKSFIGEFMESLQDPDLNVRRATLAFFNSAVHNKPSLVRDLLDDILPLLYQETKIRRDLIREVEMGPFKHTVDDGLDVRKAAFECMYSLLESCLGQLDICEFLNHVEDGLKDHYDIRMLTFIMVARLATLCPAPVLQRVDRLIEPLRATCTAKVKAGSVKQEFEKQDELKRSAMRAVAALLTIPEVGKSPIMADFSSQIRSNPELAALFESIQKDSTSAPSTDSMELS

SEQ ID NO: 48 - protein, 357 aa, Homo sapiens
  misc_feature: Incyte ID No 7435171CD1
MPEPGPDAAGTASAQPQPPPPPPPAPKESPFSIKNLLNGDHHRPPPKPQPPPRTLFAPASAAAAAAAAAAAAAKGALEGAAGFALSQVGDLAFPRFEIPAQRFALPAHYLERSPAWWYPYTLTPAGGHLPRPEASEKALLRDSSPASGTDRDSPEPLLKADPDHKELDSKSPDEIILEESDSEESKKEGEAAPGAAGASVGAAAATPGAEDWKKGAESPEKKPACRKKKTRTVFSRSQVFQLESTFDMKRYLSSSERAGLAASLHLTETQVKIWFQNRRNKWKRQLAAELEAANLSHAAAQRIVRVPILYHENSAAEGAAAAAAGAPVPVSQPLLTFPHPVYYSHPVVSSVPLLRPV

SEQ ID NO: 49 - protein, 168 aa, Homo sapiens
  misc_feature: Incyte ID No 7499936CD1
MDTKHFLPLGWNELLIASFSHRSIAVKDGILLATGLHVHRNSAHSAGVGAIFDRVLTELVSKMRDMQMDKTELGCLRAIVLFNPDSKGLSNPAEVEALREKVYASLEAYCKHKYPEQPGRFAKLLLRLPALRSIGLKCLEHLFFFKLIGDTPIDTFLMEMLEAPHQMT

SEQ ID NO: 50 - protein, 142 aa, Homo sapiens
  misc_feature: Incyte ID No 7504125CD1
MESAITLWQFLLQLLLDQKHEHLICWTSNDGEFKLLKAEEVAKLWGLRKNKTNMNYDKLSRALRYYYDKTPNGLLLTPSPLLSSIHFWSSLSPVAPLSPARLQGPSTLFQFPTLLNGHMPVPIPSLDRAASPVLLSSNSQKS

SEQ ID NO: 51 - protein, 477 aa, Homo sapiens
  misc_feature: Incyte ID No 7505742CD1
MLDMGDRKEVKMIPKSSFSINSLVPEAVQNDNHHASHGHHNSHHPQHHHHHHHHHHHPPPPAPQPPPPRAAQQQQPPPPPLAPQAGGAAQSNDEKGPQLLLLPPTDHHRPPSGAKAGGCCRPGELAPVGPDEKEKGAGAGGEEKKGAGEGGKDGEGGKEGEKKNGKYEKPPFSYNALIMMAIRQSPEKRLTLNGIYEFIMKNFPYYRENKQGWQNSIRHNLSLNKCFVKVPRHYDDPGKGNYWMLDPSSDDVFIGGTTGKLRRRSTTSRAKLAFKRGARLTSTGLTFMDRAGSLYWPMSPFLSLHHPRASSTLSYNGTTSAYPSHPMPYSSVLTQNSLGNNHSFSTANGLSVDRLVNGEIPYATHHLTAAALAASVPCGLSVPCSGTYSLNPCSVNLLAGQTSYFFPHVPHPSMTSQSSTSMSARAASSSTSPQAPSTLPCESLRPSLPSFTTGLSGGLSDYFTHQNQGSSSNPLIH

SEQ ID NO: 52 - protein, 1274 aa, Homo sapiens
  misc_feature: Incyte ID No 7505757CD1
MSTAAFHISSLLEKMTSSDKDFRCKPPHINRAWDPRSWLQIPAAVTHWPSAQQSPPPSLCRFMATSDLMSELQKDSIQLDEDSERKVVKMLLRLLEDKNGEVQNLAVKCLGPLVVKVKEYQVETIVDTLCTNMRSDKEQLRDIAGIGLKTVLSELPPAATGSGLATNVCRKITGQLTSAIAQQEDVAVQLEALDILSDMLSRLGVPLGAFHASLLHCLLPQLSSPRLAVRKRAVGALGHLAAACSTDLFVELADHLLDRLPGPRVPTSPTAIRTLIQCLGSVGRQAGHRLGAHLDRLVPLVEDFCNLDDDELRESCLQAFEAFLRKCPKEMGPHVPNVTSLCLQYIKHDPNYNYDSDEDEEQMETEDSEFSEQESEDEYSDDDDMSWKVRRAAAKCIAALISSRPDLLPDFHCTLAPVLIRRFKEREENVKADVFTAYIVLLRQTQPPKGWLEAMEEPTQTGSNLHMLRGQVPLVVKALQRQLKDRSVRARQGCFSLLTELAGVLPGSLAEHMPVLVSGIIFSLADRSSSSTIRMDALAFLQGLLGTEPAEAFHPHLPILLPPVMACVADSFYKIAAEALVVLQELVRALWPLHRPRMLDPEPYVGEMSAVTLARLRATDLDQEVKERAISCMGHLVGHLGDRLGDDLEPTLLLLLDRLRNEITRLPAIKALTLVAVSPLQLDLQPILAEALHILASFLRKNQRALRLATLAALDALAQSQGLSLPPSAVQAVLAELPALVNESDMHVAQLAVDFLATVTQAQPASLVEVSGPVLSELLRLLRSPLLPAGVLAAAEGFLQALVGTRPPCVDYAKLISLLTAPVYEQAVDGGPGLHKQVFHSLARCVAALSAACPQEAASTASRLVCDARSPHSSTGVKVLAFLSLAEVGQVAGPGPQRELKAVLLEALGSPSEDVRAAASYALGRVGAGSLPDFLPFLLEQIEAEPRRQYLLLHSLREALGAAQPDSLKPYAEDIWALLFQRCEGAEEGTRGVVAECIGKLVLVNPSFLLPRLRKQLAAGRPHTRSTVITAVKFLISDQPHPIDPLLKSFIGEFMESLQDPDLNVRRATLAFFNSAVHNKPSLVRDLLDDILPLLYQETKIRRDLIREVEMGPFKHTVDDGLDVRKAAFECMYSLLESCLGQLDICEFLNHVEDGLKDHYDIRMLTFIMVARLATLCPAPVLQRVDRLIEPLRATCTAKVKAGSVKQEFEKQDELKRSAMRAVAALLTIPEVGKSPIMADFSSQIRSNPELAALFESIQKDSTSAPSTDSMELS

SEQ ID NO: 53 - protein, 91 aa, Homo sapiens
  misc_feature: Incyte ID No 7504126CD1
MTEWETAAPAVAETPDIKLFGKWSTDDVQINDISLQAIWLLCTGAREAAFRNIKTIAECLADELINAAKGSSNSYAIKKKDELERVAKSNR

SEQ ID NO: 54 - protein, 311 aa, Homo sapiens
  misc_feature: Incyte ID No 7504099CD1
MADGVDHIDIYADVGEEFNQEAEYGGHDQIDLYDDVISPSANNGDAPEDRDYMDTPPPVPGYGPPPGPPPPQQGPPPPPGPFPPRPPGPLGPPLTLAPPPHLPGPPPGAPPPAPHVNPAFFPPPTNSGMPTSDSRGPPPTDPYGRPPPYDRGDYGPPGREMDTARTPLSEAEFEEIMNRNRAISSSAISRAVSDASAGDYGSAIETLVTAISLIKQSKVSADDRCKVLISSLQDCLHGIESKSYGSGSRRRERSRERDHSRSREKSRRHKSRSRDRHDDYYRERSRERERHRDRDRDRDRERDREREYRHR

SEQ ID NO: 55 - protein, 110 aa, Homo sapiens
  misc_feature: Incyte ID No 7505733CD1
MTSGPQTDQPKKHLTNFKSDSQLYEDTLAGRSVLIKNLTPQTLQPRWTGPYLVIYSTPTAVRLQDPPHWVHRSRIKLCPSDSQPNPSSSSWKLQVLSPTSLKLSRISEEQ

SEQ ID NO: 56 - protein, 176 aa, Homo sapiens [sequence omitted]

SEQ ID NO: 57 - protein, 532 aa, Homo sapiens
  misc_feature: Incyte ID No 7502168CD1
MEELSSVGEQVFAAECILSKRLRKGKLEYLVKWRGWSSKHNSWEPEENILDPRLLLAFQKKEHEKEVQNRKRGKRPRGRPRKLTAMSSCSRRSKLKEPDAPSKSKSSSSSSSSTSSSSSSDEEDDSDLDAKRGPRGRETHPVPQKKAQILVAKPELKDPIRKKRGRKPLPPEQKATRRPVSLAKVLKTARKDLGAPASKLPPPLSAPVAGLAALKAHAKEACGGPSAMATPENLASLMKGMASSPGRGGISWQSSIVHYMNRMTQSQAQAASRLALKAQATNKCGLGLDLKVRTQKGELGMSPPGSKIPKAPSGGAVEQKVGNTGGPPHTHGASRVPAGCPGPQPAPTQELSLQVLDLQSVKNGMPGVGLLARHATATKGVPATNPAPGKGTGSGLIGASGATMPTDTSKSEKLASRAVAPPTPASKRDCVKGSATPSGQESRTAPGEARKAATLPEMSAGEESSSSDSDPDSASPPSTGQNPSVSVQTSQDWKPTRSLIEHVFVTCFPTTPHCIFHTNVSILLFLLVIKGR

SEQ ID NO: 58 - protein, 1492 aa, Homo sapiens
  misc_feature: Incyte ID No 7503888CD1
MSTPDPPLGGTPRPGPSPGPGPSPGAMLGPSPGPSPGSAHSMMGPSPGPPSAGHPIPTQGPGGYPQDNMHQMHKPMESMHEKGMSDDPRYNQMKGMGMRSGGHAGMGPPPSPMDQHSQGYPSPLGGSEHASSPVPASGPSSGPQMSSGPGGAPLDGADPQALGQQNRGPTPFNQNQLHQLRAQIMAYKMLARGQPLPDHLQMAVQGKRPMPGMQQQMPTLPPPSVSATGPGPGPGPGPGPGPGPAPPNYSRPHGMGGPNMPPPGPSGVPPGMPGQPPGGPPKPWPEGPMANAAAPTSTPQKLIPPQPTGRPSPAPPAVPPAASPVMPPQTQSPGQPAQPAPMVPLHQKQSRITPIQKPRGLDPVEILQEREYRLQARIAHRIQELENLPGSLAGDLRTKATIELKALRLLNFQRQLRQEVVVCMRRDTALETALNAKAYKRSKRQSLREARITEKLEKQQKIEQERKRRQKHQEYLNSILQHAKDFKEYHRSVTGKIQKLTKAVATYHANTEREQKKENERIEKERMRRLMAEDEEGYRKLIDQKKDKRLAYLLQQTDEYVANLTELVRQHKAAQVAKEKKKKKKKKKAENAEGQTPAIGPDGEPLDETSQMSDLPVKVIHVESGKILTGTDAPKAGQLEAWLEMNPGYEVAPRSDSEESGSEEEEEEEEEEQPQAAQPPTLPVEEKKKIPDPDSDDVSEVDARHIIENAKQDVDDEYGVSQALARGLQSYYAVAHAVTERVDKQSALMVNGVLKQYQIKGLEWLVSLYNNNLNGILADEMGLGKTIQTIALITYLMEHKRINGPFLIIVPLSTLSNWAYEFDKWAPSVVKVSYKGSPAARRAFVPQLRSGKFNVLLTTYEYIIKDKHILAKIRWKYMIVDEGHRMKNHHCKLTQVLNTHYVAPRRLLLTGTPLQNKLPELWALLNFLLPTIFKSCSTFEQWFNAPFAMTGEKVDLNEEETILIIRRLHKVLRPFLLRRLKKEVEAQLPEKVEYVIKCDMSALQRVLYRHMQAKGVLLTDGSEKDKKGKGGTKTLMNTIMQLRKICNHPYMFQHIEESFSEHLGFTGGIVQGLDLYRASGKFELLDRILPKLRATNHKVLLFCQMTSLMTIMEDYFAYRGFKYLRLDGTTKAEDRGMLLKTFNEPGSEYFIFLLSTRAGGLGLNLQSADTVIIFDSDWNPHQDLQAQDRAHRIGQQNEVERLTCEEEEEKMFGRGSRHRKEVDYSDSLTEKQWLKAIEEGTLEEIEEEVRQKKSSRKRKRDSDAGSSTPTTSTRSRDKDDESKKQKKRGRPPAEKLSPNPPNLTKKMKKIVDAVIKYKDSSSGRQLSEVFIQLPSRKELPEYYELIRKPVDFKKIKERIRNHKYRSLNDLEKDVMLLCQNAQTFNLEGSLIYEDSIVLQSVFTSVRQKIEKEDDSEGEESEEEEEGEEEGSESESRSVKVKIKLGRKEKAQDRLKGGRRRPSRGSRAKPVVSDDDSEEEQEEDRSGSGSEED

SEQ ID NO: 59 - DNA, 5007 bp, Homo sapiens
  misc_feature: Incyte ID No 7503848CB1
gccggccggccagggggtcgcgggtatggccgaggccaggaagcggcgggagctacttcccctgatctaccaccatctgctgcgggctggctatgtgcgtgcggcgcgggaagtgaaggagcagagcggccagaagtgtttcctggctcagcccgtaacccttctggacatctatacacactggcaacaaacctcagagcttggtcggaagcggaaggcagaggaagatgcggcactgcaagctaagaaaacccgtgtgtcagaccccatcagcacctcggagagctcggaagaggaggaagaagcagaagccgaaaccgccaaagccaccccaagactagcatctaccaactcctcagtcctgggggcggacttgccatcaagcatgaaagaaaaagccaaggcagagacagagaaagctggcaagactgggaattccatgccacaccctgccactgggaagacggtggccaaccttctttctgggaagtctcccaggaagtcagcagagccctcagcaaatactacgttggtctcagaaactgaggaggagggcagcgtcccggcctttggagctgctgccaagcctgggatggtgtcagcgggccaggccgacagctccagcgaggacacctccagctccagtgatgagacagacgtggaggtaaaggcctctgaaaaaattctccaggtcagagctgcctcagcccctgccaaggggacccctgggaaaggggctaccccagcaccccctgggaaggcaggggctgtagcctcccagaccaaggcagggaagccagaggaggactcagagagcagcagcgaggagtcatctgacagtgaggaggagacgccagctgccaaggccctgcttcaggcgaaggcctcaggaaaaacctctcaggtcggagctgcctcagcccctgccaaggagtcccccaggaaaggagctgccccagcgccccctgggaagacagggcctgcagttgccaaggcccaggcggggaagcgggaggaggactcgcagagcagcagcgaggaatcggacagtgaggaggaggcgcctgctcaggcgaagccttcagggaaggccccccaggtcagagccgcctcggcccctgccaaggagtcccccaggaaaggggctgccccagcacctcctaggaaaacagggcctgcagccgcccaggtccaggtggggaagcaggaggaggactcaagaagcagcagcgaggagtcagacagtgacagagaggcactggcagccatgaatgcagctcaggtgaagcccttggggaaaagcccccaggtgaaacctgcctctaccatgggcatggggcccttggggaaaggcgccggcccagtgccacccgggaaggtggggcctgcaaccccctcagcccaggtggggaagtgggaggaggactcagagagcagtagtgaggagtcatcagacagcagtgatggagaggtgcccacagctgtggccccggctcaggaaaagtccttggggaacatcctccaggccaaacccacctccagtcctgccaaggggccccctcagaaggcagggcctgtagccgtccaggtcaaggctgaaaagcccatggacaactcggagagcagcgaggagtcatcggacagtgcggacagtgaggaggcaccagcagccatgactgcagctcaggcaaaaccagctctgaaaattcctcagaccaaggcctgcccaaagaaaaccaataccactgcatctgccaaggtcgcccctgtgcgagtgggcacccaagccccccggaaagcaggaactgcgacttctccagcaggctcatccccagctgtggctgggggcacccagagaccagcagaggattcttcaagcagtgaggaatcagatagtgaggaagagaagacaggtcttgcagtaaccgtgggacaggcaaagtctgtggggaaaggcctccaggtgaaagcagcctcagtgcctgtcaaggggtccttggggcaagggactgctccagtactccctgggaagacggggcctacagtcacccaggtgaaagctgaaaagcaggaagactctgagagcagtgaggaggaatcagacagtgaggaagcagctgcatctccagcacaggtgaaaacctcagtaaagaaaacccaggccaaagccaacccagctgccgccagagcaccttcagcaaaagggacaatttcagcccctggaaaagttgtcactgcagctgctcaagccaagcagaggtctccatccaaggtgaagccaccagtgagaaacccccagaacagtaccgtcttggcgaggggcccagcatctgtgccatctgtggggaaggccgtggctacagcagctcaggcccagacagggccagaggaggactcagggagcagtgaggaggagtcagacagtgaggaggaggcggagacgctggctcaggtgaagccttcagggaagacccaccagatcagagctgccttggctcctgccaaggagtcccccaggaaaggggctgccccaacacctcctgggaagacagggccttcggctgcccaggcagggaagcaggatgactcagggagcagcagcgaggaatcagacagtgatggggaggcaccggcagctgtgacctctgcccaggtgattaaaccccctctgatttttgtcgaccctaatcgtagtccagctggcccagctgctacacccgcacaagcccaggctgcaagcaccccgaggaaggcccgagcctcggagagcacagccaggagctcctcctccgagagcgaggatgaggacgtgatccccgctacacagtgcttgactcctggcatcagaaccaatgtggtgaccatgcccactgcccacccaagaatagcccccaaagccagcatggctggggccagcagcagcaaggagtccagtcggatatcagatggcaagaaacaggagggaccagccactcaggttgacagtgctgtgggaacactccctgcaacaagtccccagagcacctccgtccaggccaaagggaccaacaagctcagaaaacctaagcttcctgaggtccagcaggccaccaaagcccctgagagctcagatgacagtgaggacagcagcgacagttcttcagggagtgaggaagatggtgaagggccccagggggccaagtcagcccacacgctggtaggtcccaccccctccaggacagagaccctggtggaggagaccgcagcagagtccagcgaggatgatgtggtggcgccatcccagtctctcctctcaggttatatgacccctggactaaccccagccaattcccaggcctcaaaagccactcccaagctagactccagcccctcagtttcctctactctggccgccaaagatgacccagatggcaagcaggaggcaaagccccaacaggcagcaggcatgttgtcccctaaaacaggtggaaaagaggctgcttcaggcaccacacctcagaagtcccggaagcccaagaaaggggctgggaacccccaagcctcaaccctggcgctgcaaagcaacatcacccagtgcctcctgggccaaccctggcccctgaatgaggcccaggtgcaggcctcagtggtgaaggtcctgactgagctgctggaacaggaaagaaagaaggtggtggacaccaccaaggagagcagcaggaagggctgggagagccgcaagcggaagctatcgggagaccagccagctgccaggacccccaggagcaagaagaagaagaagctgggggccggggaaggtggggaggcctctgtttccccagaaaagacctccacgacttccaaggggaaagcaaagagagacaaagcaagtggtgatgtcaaggagaagaaagggaaggggtctcttggctcccaaggggccaaggacgagccagaagaggagcttcagaaggggatggggacggttgaaggtggagatcaaagcaacccaaagagcaagaaggagaagaagaaatccgacaagagaaaaaaagacaaagaaaaaaaagaaaagaagaagaaagcaaaaaaggcctcaaccaaagattctgagtcaccgtcccagaagaaaaagaagaaaaagaagaagacagcagagcagactgtatgacgagcaccagcaccaggcacagggatttcctagccgagcagtggccatccccatgcctctgacctccaccgacctctgcccaccatgggttggaactaaactgttaccttccctcgctccacagaagaagacagccagcttcaggggtccctgtgctggccaagccagtgagcctgcggggaggctggtccaaggagaaagtggaccagctcccatgacctcaccccactcccccaacacaggacgcttcatatagatgtgtacagtatatgtatttttttaagtgacctcctctccttccacagaccccacatgcccaaaggcctcgggacttcccaccaccttgctccacagatccagctaggcctgacctgtgcctcatcccgtgccgctcggtctctggctgatcccgaggctttgtcttcctctcgtcagttcttttggttgtgttttttgttttttttttaataactcaaaaaaaaaataaaagacttggaggaagggtgcaagctcccagtgcatctggggcacatgtttcttggaagggactgtctcgccgacactcgggattccctcttgcctgcatgttgaggctatgggtgaccgtggttagtgggacaggcagtgagcagtgaagtgcctgagtgccgaattggtggggaaggcttcctggatgaggtggcatctaaactaaaggatggaactaaaggatggtcatggttgatttcctgttgccagagtaagtcatgtaaatacccaaagacaagaaatggaaggggctgagaatcctcgagttaggcctcgagctaggggcaggctccctggaggtgcctggtgaacagctgtggcaggcacag

SEQ ID NO: 60 - DNA, 3118 bp, Homo sapiens
  misc_feature: Incyte ID No 2608080CB1
gcgggacttccggcgtccagtcttcgtccgccgttaggttgcggctgctgtggttgccaacgctacactgggtagaacgccagacaggggccacttttcgagaccgagtagagacggacagtgaggaggataggacccacttacacgcttttatgtcagccgcgatcccacccccacaagcgtctgcaaaacccttcctggggccttggcgacagccctgtgtcctcacggcgcgcagaggcggcgccgcgaaccttgtgtgcattttacacgacggcggggactgaggtccctcgcagcccagagccggagcccggggtcggccgagcccgcaggaccggcttcctcgccgactctcacggcctcacccagccccctgggcccatggcggcgccggcgttggcattagtatcatttgaaaacgtggttgtgaccttcactggagaggaatgggggcacctggacctggcccagaggaccctgtaccaggaggtgatgctggagacctgcaggctcctggtctcactggggcatcctgttcccaaaccagagctgatctatctactggaacatggacaggaactgtggacagtgaagagaggcctctcccaaagcacctgcgcaggtgaaaaagcaaaacccaagattacagagcctactgcttctcagctggccttctctgaggaatcctctttccaggaacttctggcacagagatcctcaagagattccaggttggggcaagctagagatgaggaaaagctaataaaaattcaggaagggaacttgaggccaggaacaaacccccacaaagagatatgccctgagaagttgagttataaacatgatgatttggagccagatgatagtctgggcttaagggttttacaggaacgagtcactccacaagatgctctccatgagtgtgactctcaaggaccaggaaaagaccccatgactgatgcaaggaataacccttacacatgcacggaatgtgggaaagggtttagcaagaagtgggcccttgttcggcatcaacagattcatgctggagtgaagccctatgagtgcaatgagtgtgggaaagcctgtcgttatatggctgatgtcattcgacatatgaggcttcatactggggaaaaaccatacaagtgtattgagtgtgggaaagccttcaaacgcaggtttcacctcacggagcaccagcgtattcacaccggagataagccctatgagtgcaaagaatgtggcaaagcattcacccaccgctcttcttttatccagcataatatgactcacactcgagaaaaaccctttttatgcaaagaatgtgggaaagctttttactacagctcctcatttgctcaacatatgaggattcatactggaaagaaactctatgagtgcggtgaatgtggaaaggccttcacgcaccgctccacatttatccagcacaatgtgacccacacaggagaaaaaccatttttatgtaaagaatgtggaaaaaccttttgcctcaactcatccttcactcagcacatgaggattcacactggagagaaaccctatgagtgcggtgaatgtggaaaggcctttactcatcgctccactttcatccgacataagaggacccataccggagagaagccctttgagtgcaaagaatgtgggaaggccttttgtgacagctcttccttaattcaacatatgaggattcacactggtgagaagccttatgagtgcagtgaatgtggaaaggcttttacacaccactctgtttttattcgacataataggacccacagtggacaaaaacccttggagtgcaaagaatgtgcaaaagccttttactatagctcttccttcactcgacacatgaggattcacactggagagaagccctatgtttgtagagaatgtggaaaggcttttacccaacctgcaaattttgttcggcataataggatccacactggagaaaaaccttttgaatgcaaagaatgtgagaaagccttctgtgacaactttgctttaactcagcacatgagaactcacactggagagaaaccctttgaatgcaatgaatgtggaaagaccttcagccacagttcatcgttcactcaccatcgaaagattcataccagagtttaaaagatacaggaaggcctattacaagtgtgtttgtctttagtgaactcatagtggtgacatacctttccttttgagtataactattgagggaaatattttggccagagagatgtcttagtatctgataatttatactaactagaaacagaattgtccctgtgaaatctttttatggcaggtaatcacttgtcatccaaagaattatattaaagattcacccagtttagagccttacactgtacctgagaataatcgaggggagagacctggtggttaacatgcatttaggtaaaatgtcaggaacaactttcctcttattaatactataaatccatctcaagcatattttaaaataatgagagatggaggaaacaatctagaagataaaagaaaaagatgtgcataacttgttttcagtttccctgtttatgacagtttgtcatttggcgggttgggggatggagcagggaataagacattgaaaaagtctcatcccctttgtgtgttttacatatatatatctctaagtccagagccaggagagttggaccattgagggcagctctgccaacatttagtaaaagtattcattgttccaaaactgtcatctctacccttgagggagtgtgtctttgtgagaaatataaaggcttgagtcatgaaatacttaacacatgtgcacatgtacacatgtacatttatatgcagtgaattgttactcgtggtagttaggttctgtgaattttctgtgcacactgaattagtgaatgctgaatgattgttgctaggggaaatacagcgttagcttctgtgagcctctgatccaaacatttttatcaaccagtcaacacataaccttcttctatgggtatttgtttgtttgtttgtttaaagacactttatcagtgtttcttgaataattataattattctttataataaaaatattttaattatagcacaaaaa

SEQ ID NO: 61 - DNA, 2909 bp, Homo sapiens
  misc_feature: Incyte ID No 7503402CB1
atcctggtgcatggtggtcggacgaaggaattgttggaaaattttctcggaggtagaagatgttgttagcccaaataaatcgagattctcagggaatgacagagtttcctggaggagggatggaggcgcaacatgttacgctgtgcttgacagaggcagtcaccgtggcagatgcaaaactcatagatggccaggtcattcagttggaagatggttctgcggcctatgttcaacatgtacccatacctaaaagtacaggggacagtttgcgtctagaggatggtcaagcagtacagttagaagatggtaccacagcatttattcaccacacctccaaagatagttatgaccagagtgcattacaggcggttcagctggaagatggtaccacagcttatatccaccatgcagtgcaagtcccgcagtctgacaccatcttggcaattcaggctgatgggacagtggcaggtctgcacactggggatgctacaattgaccctgacaccatcagtgctttggaacagtatgcagcaaaggtgtccattgatggaagtgaaagtgtagcaggtactggaatgattggagaaaatgagcaagagaaaaaaatgcagattgttttacaaggacatgctacaagagtaactgctaaatctcaacagagtggagagaaggcatttcgatgtgaatatgatggatgtggaaaattatatacaacagctcatcatctcaaggtccatgagaggtcacacacaggagatcggccttatcagtgtgagcatgcaggctgtgggaaggcatttgcaacaggttatggattaaaaagtcacgtcagaactcatacaggagaaaagccatatcggtgttcggaagataattgtactaaatctttcaaaacttcaggagatctacagaaacacatcagaactcatacaggagaaaggccctttaagtgtcccttcgaaggctgcggtcggtcctttacaacatcaaatatcagaaaagtgcacgttaggacacacacaggagaaagaccttattactgcacagagccaggatgtgggagggcatttgccagtgcaacaaattataaaaaccatgtgaggatacacacaggagaaaagccatatgtttgtacagttcctgggtgtgacaaaaggtttacagaatattccagtttgtacaaacatcatgttgtccacactcattccaaaccttacaactgtaaccactgtgggaagacatacaagcagatctccacgctggccatgcacaaacggacagcccacaacgacactgagcccatcgaggaggagcaggaagccttctttgagccgcccccaggtcaaggtgaagatgttcttaaagggtcccagattacgtatgttacaggtgtagaaggggacgacgttgtttctacacaagtagccacagtaacccaatctggactgagtcaacaagttacactcatatcccaggatgggactcagcatgtcaacatatctcaagctgacatgcaggccattggcaacaccatcacaatggtaacgcaggatggcacgcccatcacagtccccgcccatgatgcagtcatctcctcagcaggaacgcactctgttgctatggttactgctgagggtacagaagggcaacaggttgcaattgtagctcaagacttggcagcattccatactgcctcatcagaaatggggcaccagcagcatagccatcacttagtaaccacagaaaccagacctctgaccttagtagcaacatccaatggcacccagattgcagttcagcttggagaacagccatctctggaagaagccatcagaatagcgtctagaatccaacaaggagaaacgccagggttggatgattaatcctcagaacaatggagcaataaagcagaaggagtctttcatcttctggcagcagaaatccatgaagcccgggcccaggaaaattagaagttttccattcctgatacactgtacacatttttatgcgagagtggagaacattttattcttgacacttttgtgtatataacccttggaatagattctcagagtgattcattgtgtacaaggaagtatgaaattagggcaatacagtaaattttcatgttactcttttatcagatcacaaactcctagagtctacatgcaagactagtaaagtcttatggagtcttatgatggatttttaacttcccgtggaaaaaaaaataaaggctgtatctaaaatatcaaaggttctatatgtcacacaatcgtaattccaaaagccattatggataataaagggtgtaaagccttcagatatttccccagttagtagagtgtctgcggtttttgttctactatatgcttgtccatttttatttgtatctcatggtttgcagactgtttgaataatttatagtttcccatccctgttaaaaaccagctcttcaagctgaaatgctaattatattggcattacattgaattatgtacaaaattataaaatttggttatttaaaattaaaaagttaaatccagtggttttgttaaagattttgcttagtattcaatttttattactgttttttaaaaataatgaatcatcaaagtttaaccacaggctggtgcccgggataacagtactgtaattggaaatggctttactctgaaaattaggttagtgggttggtgtaaattatttatttttgcttatgtacttttgttttaaagcttatttaccccaaagtttattattaattttgaatacagcaatttttaaaatgttacttttatatttatttatttatcatattggtggggggttggggggaaatggtgcagaaaatatcgcaaatgtaatggaatgtaacctaatacacatattgaaaggacaaaaaaaaa

SEQ ID NO: 62 - DNA, 1613 bp, Homo sapiens
  misc_feature: Incyte ID No 7503517CB1
gcttcccctccgtcatccatcgctcggcggtcttctcttctccatgggtctgctctgcgcgttccatcgagtttctcggccatcgcgcgcctgcgccattgggctgtcagtcagaggcggcgtggagatcgctgggagcggttgcggcgtgcggggagctgagttatagctgtgacttctgccctgccaggccgcacacaagctggctgacccggtttgtaaaaatggaatttcaagcagtagtgatggcagtaggtggaggatctcggatgacagacctaacttccagcattcccaaacctctgcttccagttgggaacaaacctttaatttggtacccattgaacctgcttgagcgtgttggatttgaagaagtcattgtggttacaaccagggatgttcaaaaggctctatgtgcagaattcaagatgaaaatgaagccagatattgtgtgtattcctgatgatgctgacatgggaactgcagattctttgcgctacatatatccaaaacttaagacagatgtgctggtgctgagctgtgatctgataacagacgttgccttacatgaggttgtggacctgtttagagcttatgatgcatcacttgctatgttgatgagaaaaggccaagatagcatagaacctgttcccggtcaaaaggggaaaaaaaaagcagtggagcagcgtgacttcattggagtggacagcacaggaaagaggctgctcttcatggctaatgaagcagacttggatgaagagctggtcattaagggatccatcctacagaagtcaataacttctatccggagtgaactgattccatatttagtgagaaaacagttttcctcagcttcctcacaacagggacaagaagaaaaagaggaggatctaaagaaaaaggagctgaagtccttagatatctacagttttataaaagaagccaatacactgaacctggctccctatgatgcctgctggaatgcctgtcgaggagacaggtgggaagacttgtccagatcacaggtgcgctgctatgtccacatcatgaaagaggggctctgctctcgagtgagcacactgggactctacatggaagcaaacagacaggtgcccaaattgctgtctgctctctgtccagaagaaccaccagtccattcgtcagcccagattgtcagcaaacacctggttggagttgacagcctcattgggccagagacacagattggagagaagtcatccattaagcgctcagtcattggctcatcctgtctcataaaagatagagtgactattaccaattgccttctcatgaactcagtcactgtggaggaaggaagcaatatccaaggcagtgtcatctgcaacaatgctgtgatcgagaagggtgcagacatcaaggactgcttgattggaagtggccagaggattgaagccaaagctaaacgagtgaatgaggtgatcgtggggaatgaccagctcatggagatctgagttctgagcaagtcagactccttccttttggcctccaaagccacagatgttggccggcccacctgtttaactctgtatttatttcccaataaagaagggcttccaaaggcaaaaaaaaaa

SEQ ID NO: 63 - DNA, 1022 bp, Homo sapiens
  misc_feature: Incyte ID No 7500014CB1
gttcggttgcgctgcggagcgcagctgtgagggagtcgctgtgatccggggccccggaacccgagctggagctgaagcgcaggctgcggggcgcggagtcgggagtgcaggcctgagtgttccttccagcatgtcggagggggagtcccagacagtacttagcagtggctcagacccaaaggtagaatcctcatcttcagctcctggcctgacatcacctgtcgtgcccccctctgtcaagactccgacacctgaaccagctgaggtggagactcgcaaggtggtgctgatgcagtgcaacattgagtcggtggaggagggagtcaaacaccacctgacacttctgctgaagttggaggacaaactgaaccggcacctgagctgtgacctgatgccaaatgagaatatccccgagttggcggctgagctggtgcagctgggcttcattagtgaggctgaccagagccggttgacttctctgctagaagagaccttgaacaagttcaattttgccaggaacagtaccctcaactcagccgctgtcaccgtctcctcttagagctcactcgggccaggccctgatctgcgctgtggctgtccctggacgtgctgcagccctcctgtcccttccccccagtcagtattaccctgtgaagccccttccctcctttattattcaggagggctgggggggctccctggttctgagcatcatcctttcccctcccctctcttcctcccctctgcactttgtttacttgttttgcacagacgtgggcctgggccttctcagcagccgccttctagttgggggctagtcgctgatctgccggctcccgcccagcctgtgtggaaaggaggcccacgggcactaggggagccgaattctacaatcccgctggggcggccggggcgggagagaaaggtggtgctgcagtggtggccctggggggccattcgattcgcctcagttgctgctgtaataaaagtctactttttgctaaaaaaaaaaaaaaaaaaaa

SEQ ID NO: 64 - DNA, 1816 bp, Homo sapiens
  misc_feature: Incyte ID No 7501365CB1
gggcatggctcgggtggcgtgggggctgctgtggttgctgctgggcagcgccggggcgcagtacgagaagtacagcttccggggcttcccgcccgaggacctgatgccgctggccgcggcgtacgggcacgctctggagcagtacgagggagagagctggcgcgagagcgcgcgctacctggaggcggcgctgcggctgcaccggctcctgcgcgacagcgaggccttctgccacgccaactgcagcggccccgcgcccgcggccaagcccgatcccgacggcggccgcgcagacgagtgggcctgcgagctgcggctcttcggccgcgtcctggagcgagccgcctgcctgcggcgctgcaagcggacgctgcccgccttccaggtgccctacccgccgcggcagctgctgcgtgacttccagagccgcctgccctaccagtacctgcactacgcgctgttcaaggctaaccggctggagaaggcggtggcggcggcctacaccttcctccagaggaacccgaagcacgagctgaccgccaagtatctcaactactatcgggggatgctggacgtcgccgacgagtccctcacggacctagaggcccagccctacgaggccgtgttcctccgggctgtgaagctctacaacagcggggatttccgcagcagcacggaggacatggagcgggccttgtcagagtacctggcagtctttgcccggtgcctggccggctgtgaaggggcccatgagcaggtggacttcaaggacttctacccggccatagcagatctctttgcagagtccctgcagtgcaaggtggactgtgaggccaatttgacccccaatgtgggtggctacttcgtggacaagttcgtggccaccatgtaccactacctgcagtttgcctactataagttgaatgatgtgcgccaggctgcccgcagcgccgccagctacatgctcttcgaccccaaggacagcgtcatgcagcagaacctggtgtattaccggttccaccgggctcgctggggcctggaagaggaggacttccagccccgggaggaggccatgctctaccacaaccagaccgccgagctgcgggagctgctggagttcacccacatgtacctgcagtcagatgatgagagccagagcctgaactcgcatgagaaggggacaccccacacccctcaagcttgggaagcctggtgccgatggccccaccctcaccagcctgggcagcagcaagaactatttattaaaaacttaagatgggccaggtgcggtggctcacacctgtaatcccagcattttgggaggccaaggtgggtggatcacttgaggccaggagttcaagaccagcctggccaacatgatgagacctccgtctctactaaaatacataaattagccgggtgtggtggcaggcgcctgaaatcccagctactcaagaggctgaggcaggagaatcgcttgaacctgggaggcaaaggttgcagtgaactgagattgcgccaccgcactccagcctgggcgacagagcgagactccatctttaaaaaaaaacaagacgggccggcacggtggctcacgcctgtaatcccagcactgagaggccgatcacttgaggtcaggagttcaagaccagcctggccaacatggtgaaaccccatctctactaaaaaatacaaaaattagccaggcatggtggcacacacctgtaatcgtagctgaggcaggagaatcgcctgaacccaggaggcggagcttgcagtgagccgagatcattaccctgcagtgcataataagtct

SEQ ID NO: 65 - DNA, 5955 bp, Homo sapiens
  misc_feature: Incyte ID No 7503540CB1
ttcaaagacttagaagctaagcagaaaatgagcttaacatcctggtttttggtgagcagtggaggcactcgccacaggctgccacgagaaatgatttttgttggaagagatgactgtgagctcatgttgcagtctcgtagtgtggataagcaacacgctgtcatcaactatgatgcgtctacggatgagcatttagtgaaggatttgggcagcctcaatgggacttttgtgaatgatgtaaggattccggaacagacttatatcaccttgaaacttgaagataagctgagatttggatatgatacaaatcttttcactgtagtacaaggagaaatgagggtccctgaagaagctcttaagcatgagaagtttaccattcagcttcagttgtcccaaaaatcttcagaatcagaattatccaaatctgcaagtgccaaaagcatagattcaaaggtagcagacgctgctactgaagtgcagcacaaaactactgaagcactgaaatccgaggaaaaagccatggatatttctgctatgccccgtggtactccattatatgggcagccgtcatggtggggggatgatgaggtggatgaaaaaagagctttcaagacaaatggcaaacctgaagaaaaaaaccatgaagctggaacatcagggtgcagcatagatgccaagcaagttgaggaacaatctgcagctgcaaatgaagaagtactttttcctttctgtagggaaccaagttattttgaaatccctacaaaagaattccagcaaccatcacaaataacagaaagcactattcatgaaatcccaacaaaagacacgccaagttcccatataacaggtgcagggcatgcttcatttaccattgaatttgatgacagtaccccagggaaggtaactattagagaccatgtgacaaagtttacttctgatcagcgccacaagtccaagaagtcttctcctggaactcaagacttgctggggattcaaacaggaatgatggcacccgaaaacaaagttgctgactggctagcacaaaacaaccctcctcaaatgctatgggaaagaacagaagaggattctaaaagcattaaaagtgatgttccagtgtacttgaaaaggttgaaaggaaataaacatgatgatggtacgcaaagtgattcagagaacgctggggctcacaggcgctgtagcaaacgtgcaactcttgaggaacacttaagacgccaccattcagaacacaaaaagctacagaaggtccaggctactgaaaagcatcaagaccaagctgttgtgtttggagtagatgacaatcaggattataataggcctgttatcaacgaaaaacataaagatctaataaaagattgggctctcagttctgctgcagcagtaatggaagaaagaaaaccactgactacatctggatttcaccactcagaggaaggcacatcttcatctggaagcaaacgttgggtttcacagtgggctagtttggctgccaatcatacaaggcatgatcaagaagaaaggataatggaattttctgcacctcttcctttagagaatgagacagagatcagtgagtctggcatgacagtgagaagtactggctctgcaacttccttggctagccagggagagagaaggagacgaactcttccccagcttccaaatgaagaaaagtctcttgagagccacagagcaaaggttgtaacacagaggtcagagataggagaaaaacaagacacagaacttcaggagaaagaaacacctacacaggtataccagaaagataaacaagatgctgacagacccttgagtaaaatgaacagggcagtaaatggagagactctcaaaactggtggagataataaaaccctacttcacttaggcagctctgctcctggaaaagagaaaagtgaaactgataaggaaacttctttggtaaagcaaacattagcaaaacttcaacaacaagaacaaagggaggaggctcagtggacacctactaaattgtcttccaaaaatgtttcaggtcagacagataaatgtagggaggaaacttttaaacaagaatcacaacctccagaaaaaaattcaggacattctacaagcaaaggagacagagtggcacaaagtgagagcaagagaagaaaagctgaggaaattctgaaaagtcagactccaaagggaggagacaagaaggaatcctccaagtcattagtgcgacaagggagcttcactatagaaaaacccagcccaaacatacccatagaacttattccccatataaataaacagacttcctctactccttcttctttagcattaacatctgcaagtagaatacgagaaagaagtgagtctttggatcctgattctagtatggacacaacccttattctaaaagacacagaagcagtaatggcttttctagaagctaaactacgtgaagataataaaactgatgaaggaccagatactcccagttataatagagacaattctatttcaccagaatctgatgtagatacagctagtacaatcagtctggttactggagaaactgaaagaaagtcaacccaaaagcgaaagagtttcactagcctctataaagataggtgttccacaggttctccttccaaagatgttacaaaatcatcatcttcaggtgctagggaaaaaatggaaaagaaaacaaaaagtcgttccacagatgtgggttcaagagcagatggtcgtaaatttgttcagtccagtgggagaataagacagccctcagtagacttaacagatgatgaccaaacctctagtgtacctcattctgccatctctgatattatgtcatctgatcaagaaacttactcttgtaaacctcatggacggactccacttacctcagctgatgagcatgtacattccaaactggaaggaagtaaagtaacgaaatctaagacttctccggtggtatctggttcatctagtaaatcaaccacccttccaaggccacgacctaccaggacttccctcttgcgcagagcacgacttggtgaagcttcagacagtgaacttgctgatgctgacaaagcatctgttgcttctgaagtatccacaacaagttctacatcaaaacctcccacaggaaggcgtaacatctctcggattgatttattggctcagcctcgtagaacacgacttggctcactgtcagctcgtagtgactctgaagcaacaatttctagaagtagtgcctcttcgaggaccgcagaagccatcattagaagtggagccagactagtaccatcagataaattttctcctagaattagagctaacagtatctctcgactctcagactccaaggttaaaagtatgacctcagctcatggctctgcttcagccctgaaaaccactcgcttgcagagcgctggatcagcaatgcctactagttcttcattcaaacaccggattaaagagcaggaagactacatccgagattggactgctcatcgagaagagatagccaggatcagccaagatcttgctctcattgctcgggagatcaacgatgtagcaggagagatagattcagtgacttcatcaggcactgcccctagtaccacagtaagcactgctgccaccacccctggctctgccatagacactagagaagagttggttgatcgtgtttttgatgaaagcctcaacttccaaaagattcctccattagttcattccaaaacaccagaaggaaacaacggtcgatctggtgatccaagacctcaagcagcagagcctcccgatcacttaacaattacaaggcggagaacctggagcagggatgaagtcatgggagataatctgctgctgtcatccgtctttcagttctctaagaagataagacaatctatagataagacagctggaaagatcagaatattatttaaagacaaagatcggaattgggatgacatagaaagcaaattaagagccgaaagtgaagtccctattgtgaaaacctcaagcatggagatttcttctatcttacaggaactgaaaagagtagaaaagcagctacaagcaatcaatgctatgattgatcctgatggaactttggaggctctgaacaacatgggatttcccagtgctatgttgccatctccaccgaaacagaagtccagccctgtgaataaccaccacagcccgggtcagacaccaacacttggccaaccagaagctagggctcttcatcctgctgctgtttcagccgcagctgaatttgagaatgctgaatctgaggctgatttcagtatacatttcaatagagtcaaccctgatggggaagaggaagatgttacagtacataaatgactttctcttgattgttgaaaaatcattacctgtggaatgactaggaatattggaagcagcatagtgttgatgtacgcaaaacaagacagcttggtcagctacaatcttggaatccctgtcttcttaattttatttatttatttttgacgtataatgtagtatatcaatcctttcgaactatttagataaccacttgatgcacaaataggaaaaagcagatgtggcagtgtcgccttttgtggttttatgattttcaaattgaatttaatgattacaccctttcccttcatagatcttttttcttttttttaagccatgctgtgacctacaagcaaactaaatagccaacatttctgaacccctaagtctcctgtgccaagctgctccctgaaatggacttcttcatctgtacagatttgttaaaccattctatttgcttcttaataataggatttatattagtactcattaccattggacacaatgacataagtactctccacagtaaagcagacctttcacaacagtcactctgtgtcctaaaattttccaacatagatgtgatttatataactttgttgatacgtaaattgtcttggggtttacggaaattaactattatgtttgcactaagatttgctgggagtggtaggtggacatatctatatatcaataaggactaaccgtcttttttgtacataggagattgataatactgtatttgttttaagcccacagtgttttactccactttcaaaaagatcaatttggcactttttttcattttttttaatggaaataagatttggtctctcattttaggttaaatgataactagaaagattaaactagacagatagtttaggtggagtatatttttaaaactcagaacatgtatattggtcctgtgttaccaagtttatatgtgacagttgaaaaagaaattcccttgaaatgatcatgaggttaaaattttcttcattaggggacttggagaaccagtagtcgtaagattagttgatagtttcactcccaagcaatgaattgcttctgtgtgtttccctgtaggactcaatagtaaatgctgtctgtcttacacatttataaggaccctgcaagacgacgacaaaggcctttggcctgtgctactaaacaagaagcctatgaaaaatttcttctttaaacttgttttttctctttccagtaagttcacatttggataattttaaaaagaaaagtaattacctttgtgtttccagaacactataattggggtgtatcttaattcagttaaatattattagtagacctggattttcccccttgaccccatcagtctataaaggttaaactgcaacttttatgaaatggtctttaatatttccacaataatcctgtgctatatttgttttaagaaacaaagtaactctatacacttcaagactttacaggattttttaaatcctgtattgttggatcaattaataaagatgcaaaaaaactttatagagatgtaaaaacaaaactataatggatctcctatttttctttaaatacaaaaaaaaaaaaaaaaaa

SEQ ID NO: 66 - DNA, 3665 bp, Homo sapiens
  misc_feature: Incyte ID No 7504326CB1
ggcggggagcaggaggaggagaaggcggaggaggcagtcgctctccgcggggctgagccggacgcgtcgtcttgcccccctccccccggttcgcggtgccgccgtgtagttggcgccgctgccccggctgagagtgagcgtggtgtcgacggagggagatggcccgggagcgccggcgccagtaactgggagctgctgagagtcgccgagggcgcgccgggcccaggtgccggggctgcccgccgcccgccgccgccgccgcctgcgcgcccgcccgcctttcgcggccgctctcccccctccccgacacacactcacaggccgggcattgatggtaatgtatgcgaggaaacagcagagactcagtgatggctgtcacgaccggaggggggactcgcagccttaccaggcacttaagtattcatcgaagagtcaccccagtagcggtgatcacagacatgaaaagatgcgagacgccggagatccttcaccaccaaataaaatgttgcggagatctgatagtcctgaaaacaaatacagtgacagcacaggtcacagtaaggccaaaaatgtgcatactcacagagttagagagagggatggtgggaccagttactctccacaagaaaattcacacaaccacagtgctcttcatagttcaaattcacattcttctaatccaagcaataacccaagcaaaacttcagatgcaccttatgattctgcagatgactggtctgagcatattagctcttctgggaaaaagtactactacaattgtcgaacagaagtttcacaatgggaaaaaccaaaagagtggcttgaaagagaacagagacaaaaagaagcaaacaagatggcagtcaacagcttcccaaaagatagggattacagaagagaggtgatgcaagcaacagccactagtgggtttgccagtggaatggaagacaagcattccagtgatgccagtagtttgctcccacagaatattttgtctcaaacaagcagacacaatgacagagactacagactgccaagagcagagactcacagtagttctacgccagtacagcaccccatcaaaccagtggttcatccaactgctaccccaagcactgttccttctagtccatttacgctacagtctgatcaccagccaaagaaatcatttgatgctaatggagcatctactttatcaaaactgcctacacccacatcttctgtccctgcacagaaaacagaaagaaaagaatctacatcaggagacaaacccgtatcacattcttgcacaactccttccacgtcttctgcctctggactgaaccccacatctgcacctccaacatctgcttcagcggtccctgtttctcctgttccacagtcgccaatacctcccttacttcaggacccaaatcttcttagacaattgcttcctgctttgcaagccacgctgcagcttaataattctaatgtggacatatctaaaataaatgaagttcttacagcagctgtgacacaagcctcactgcagtctataattcataagtttcttactgctggaccatctgctttcaacataacgtctctgatttctcaagctgctcagctctctacacaagcccagccatctaatcagtctccgatgtctttaacatctgatgcgtcatccccaagatcatatgtttctccaagaataagcacacctcaaactaacacagtccctatcaaacctttgatcagtactcctcctgtttcatcacagccaaaggttagtactccagtagttaagcaaggaccagtgtcacagtcagccacacagcagcctgtaactgctgacaagcagcaaggtcatgaacctgtctctcctcgaagtcttcagcgctcaagtagccagagaagtccatcacctggtcccaatcatacttctaatagtagtaatgcatcaaatgcaacagttgtaccacagaattcttctgcccgatccacgtgttcattaacgcctgcactagcagcacacttcagtgaaaatctcataaaacacgttcaaggatggcctgcagatcatgcagagaagcaggcatcaagattacgcgaagaagcgcataacatgggaactattcacatgtccgaaatttgtactgaattaaaaaatttaagatctttagtccgagtatgtgaaattcaagcaactttgcgagagcaaaggatactatttttgagacaacaaattaaggaacttgaaaagctaaaaaatcagaattccttcatggtgtgaagatgtgaataattgcacatggttttgagaacaggaactgtaaatctgttgcccaatcttaacatttttgagctgcatttaagtagactttggaccgttaagctgggcaaaggaaatgacaaggggacggggtctgtgagagtcaattcaggggaaagatacaagattgatttgtaaaacccttgaaatgtagatttcttgtagatgtatccttcacgttgtaaatatgttttgtagagtgaagccatgggaagccatgtgtaacagagcttagacatccaaaactaatcaatgctgaggtggctaaatacctagccttttacatgtaaacctgtctgcaaaattagcttttttaaaaaaaaaaaaaaaaaaattgggggggttaatttatcattcagaaatcttgcattttcaaaaattcagtgcaagcgccaggcgatttgtgtctaaggatacgattttgaaccatatgggcagtgtacaaaatatgaaacaactgtttccacacttgcacctgatcaagagcagtgcttctccatttgttttgcagagaaatgtttttcatttcccgtgtgtttccatttccttctgaaattctgattttatccatttttttaaggctcctctttatctcctttcttaaggcactgttgctatggcacttttctataaccttttcattcctgtgtacagtagcttaaaattgcagtgattgagcataacctacttgtttgtataaattattgaaatccatttgcaccctgtaagaatggacttaaaagtactgctggacaggcatgtgtgctcaaagtacattgattgctcaaatataaggaaatggcccaatgaacgtggttgtgggaggggaaagaggaaacagagctagtcagatgtgaattgtatctgttgtaataaacatgttaaaacaaacaaaaattgttatttttcttttccttcggtcagtgcacattagcatttgaactacctggggattctttatcagaactgttcttgttgaatatttatacttaattgaaataattccttaagggaggttttgtttaaaacgtattaacaggaaattgtgtatgagatatttaatgaaataagaaattcaacaagaatgattaagtcacttcccaagtggttgtcatttgttaaaccctggtttacctgtcttgctattatgacatttcatttggaaggatgtttgtgttgtagctaactgttcaagtctggtgctgactgctgttcttagccatcacaaaacgctaaatttgtgtaattggagcttcctgctgttatctggaaatagcaggaaagcgcagctttgtatattgtttcctaaagtatattaaaataaaaaaagaaactattgctactgaaaaaa

SEQ ID NO: 67 - DNA, 1260 bp, Homo sapiens
  misc_feature: Incyte ID No 7504388CB1
ggccgcggggcagcggagggcgccggcactccggtccccgccgctccccgtccccgctgctcctagcccctgccgcgtccccggcggagcgggcatggcgccacccgcggcgcctggccgggaccgtgtgggccgtgaggatgaggacggctgggagacgcgaggggaccgcaaggtgcaggccaagctggagaacgccgaagtgctggagctgacggtgcggcgggtccagggtgtgctgcggggccgggcgcgcgagcgcgagcagctgcaggcggaagcgagcgagcgcttcgctgccggctacatccagtgcatgcacgaggtgcacacgttcgtgtccacgtgccaggccatcgacgctaccgtcgctgccgagctcctgaaccatctgctcgagtccatgccgctgcgtgagggcagcagcttccaggatctgctgggggacgccctggcggggccacctagagcccctggacggagtggctggcctgcggggggcgctccgggatccccaatacccagccccccgggtcctggggacgacctgtgctccgacctggaggaggcccctgaggctgaactgagtcaggctcctgctgaggggcccgacttggtgcccgcagccctgggcagcctgaccacagcccaaattgcccggagtgtctggaggccttggtgaccaatgccagccagagtcctgcgggggtgggcccggccctccctggatctcctccctcctcccaggggttcagatgtggtggggtagggccctggaagtctcccaggtcttccctccctcctctgatggatggcttgcagggcagcccctggtaaccagcccagtcaggccccagccccgtttcttaagaaacttttagggaccctgcagctctggagtgggtggagggagggagctacgggcaggaggaagaattttgtagagctgccagcgctctcccaggttcacccacccaggcttcaccagccctgtgcgggctctgggggcagaggtggcagaaatggtgctgggcactagtgttccaggcagccctgggctaaacaaaagcttgaacttgccacttcagcggggagatgagaggcaggtgcactgagctgcactgcccagagctgtgatgctctgtacatcttgtttgtagcacacttgagtttgtgtattccattgacatcaaatgtgacaattttactaaataaagaattttggagttagttacccttgaaaaaaaaaaaaaaaaaagg

SEQ ID NO: 68 - DNA, 3907 bp, Homo sapiens
  misc_feature: Incyte ID No 2828380CB1
aaaggccttatcgctgtgtgtgtgtttcagtctacgtggattaaacattacttcgctccgtctgcctggattaaacgtgcactttgcagtcctcacttctccgtaccagtgatctggggatcgctacggaccttaaaatacccatagccccttcgcccctgcaacaggcacttctccccacgtttgatgtgtgaataattacattgactgagaaagaaacccagaagaggaagaagaggaaggaaaaggagtcagtgatggctactcaggggcatttgacattcaaggatgtagccatagaattctctcaggaggagtggaaatgcctggagcctgtgcagaaagctttgtacaaggatgtgatgttggagaactataggaacctggtcttcctaggtatctctcctaaatgtgtgatcaaggaattaccaccaacagagaacagtaatacaggagaaaggttccaaacagtggcactggaaagacatcaaagctatgatattgaaaatttatacttcagggaaatacagaaacatctacatgaccttgaatttcaatggaaagatggtgaaacaaatgataaagaagtgccagtgccccatgaaaacaatcttactggtaaaagagatcaacatagtcaaggggatgtagaaaacaatcatattgaaaaccagcttacatcaaactttgagtcacgtctggctgaactgcagaaagttcaaactgaagggagactttatgaatgtaatgaaacggagaagacaggtaataatggttgtttagtttctccacacattagggaaaaaacgtatgtatgtaatgaatgtggcaaagcctttaaagcgtcttccagccttattaatcatcagaggatacatactacagagaaaccttacaaatgcaatgagtgtggcaaagcctttcatcgggcctcactactaactgtacacaaggtagtccatacaagagggaaatcatatcaatgtgatgtatgtggcaagatcttcagaaaaaattcatattttgtaagacaccaaaggagtcacactggacagaaaccctacatatgtaatgaatgtggcaagtcctttagtaaaagttcccaccttgcagttcatcagagaattcataccggtgaaaaaccttacaaatgtaatctgtgtgggaaatcctttagtcagcgtgtccatcttagacttcatcagacagttcatactggagagagacccttcaaatgtaatgagtgtggcaaaacctttaaacggagctcaaacctcactgtacatcaggtaatccatgcaggaaagaaaccatataaatgtgatgtatgtggcaaggcattcagacatagatcaaatcttgtatgtcaccggagaatccacagtggagagaaacaatacaaatgcaatgaatgtggcaaggtcttcagtaaacgttcaagtcttgcagtgcatcgacgaattcacactgtagagaaaccttgcaaatgcaatgaatgtggcaaggtcttcagtaaacgttcaagtcttgcagtgcatcagagaattcatactggacagaaaacttacaaatgcaataaatgtggcaaggtgtacagtaagcattcacatcttgcagtgcattggagaattcataccggcgagaaagcttataaatgcaatgaatgtggcaaagttttcagcatacattcacgacttgcagctcatcagagaattcatactggagagaaaccttacaaatgcaatgaatgtggcaaggtcttcagtcaacattcacgtcttgcagtgcatcggagaattcatactggagagaaaccttacaaatgcaaagaatgtggcaaggtcttcagtgaccgttcagcttttgcaaggcatcggagaattcatactggagagaagccttacaaatgcaaagaatgtggcaaggtcttcagtcaatgttcacgtcttacagtgcatctgagaattcatagtggagagaaaccttacaaatgcaatgaatgcggcaaggtctacagtcagtattcacatcttgtagggcatcgaagagttcatactggagagaaaccatacaaatgtcatgaatgtggcaaagcctttaatcagggctccacactcaatagacatcagagaattcataccggagagaaaccttacaaatgcaatcagtgtgggaattcctttagtcagcgtgtccatcttagacttcatcagactgttcatactggagacagaccttacaaatgtaatgagtgtggcaaaacctttaaacggagctcaaacctcactgcacatcagataattcatgcaggaaagaaaccatataaatgtgatgaatgtggcaaggtattcaggcatagttcacatcttgtaagtcaccagagaatccacactggagagaaaagatacaaatgtattgaatgtggcaaagcctttgggcggttgttttccctcagcaaacaccaaagaattcattctggcaaaaaaccttataaatgtaatgagtgtgggaaatcttttatttgtcgctcaggcctcactaaacatcgaataagacatactggagagagccttacaactaaactcaatgtgacaaggccttagacgttgtcctagtttctggaatcaccgaataattcctacttactgatataccttgtatatttaccccttctcttgaaatccctgtggaattgtaatctccagtattggaggtggggcccattgggaggtgattgaatcatggaagtggatttctcaaactgagaaagatgtagcgtcatccccttggtgctgtcctggcaatagtgacttctcttgaggtctggctgtttagaagtgcatagcacttccctgtcgcttgccctcattctcaccatgtgaaataccgacacccgctttgccttccaccatgattttaaccttcctgaggcttccctagagggtgatcagatgccagcaccatgttttcatttaagccttcagaaatatgagccaattaaactcttttctttatacattagccagcctcaggtttttttttgtagcaatgcagttatgacctaatacatttacacatgcagtaaatatacctaagttttaagctagtattcactactcactaggtgtaagaatttgtatatatattccaggatgtatacatcctggagaaaaatcacagaagtgtaatacgtgtggcaagaattctactcaaaagccagaacttgtaaatctaggtaattaaaagggcatagatgtatgaaatgaaatgagggtggcaaaacctttactagagttcaatcactacttgtcataagagagtttatactgaaaggaaatcatataaatgtatgtgtcagaggctttccccaggcattggaactcactaggcatcagaatatacatctttgagaggaaccacagaaatgtaatgtgcatgctaaggtttttacccgaacatcaaaatggaaagagtattcatactagagacaaccttccaaataaatggttttaaaaaatgaaatgttcaaagacctggaatcaacccaaatgcttatcagtgatagactgaataaagaaaatgtggtacatatacaccatgggatactatgcagccatagaaaggaatgagatcatgtccattgcagcgatgtggatggagctggaagccattatcctcaggaaactaacacaggaacagaaaatcaaacagcacatgttctcacttaaaagtggaagctgaacaatgagaacacatatacccagggaggggagcacacacactggggcctgctgatgggggtgggaaggcagggaagtggagcatcaggacaaatagctaatgcatttttgccttaatctttccagcacactgcgccgatatatcgtgatccgagctcgtccctcta

SEQ ID NO: 69 - DNA, 3313 bp, Homo sapiens
  misc_feature: Incyte ID No 6456919CB1
cgacaggggtcaggatctcggctttcttgcttcgagagggactaggtgcctccaccagagcttctgtcgctctgtaacctgcactgtgacctacactagtcgcgggagccacgcagaggacgccggaacaccctggaagccgagaaatggacccagtggcttttaaggatgtggctgtgaacttcacccaggaggagtgggctttgctggatatttcccagaggaaactctacagggaagtgatgctggaaactttcaggaacctgacctctttaggaaaaaggtggaaagaccagaacattgaatatgagcaccaaaaccccaggagaaacttcaggagtctcatagaagaaaaagtcaatgaaattaaagatgacagtcattgtggagaaacttttaccccagttccagatgacagactgaacttccaggagaagaaagcttctcctgaagtaaaatcatgtgaaagctttgtgtgtggagaagttggcctaggtaactcatcttttaatatgagcatcagaggtgacattggacacaaggcatatgagtatcaggaatatggaccaaagccatgtaagtgtcaacaacctaaaaaagccttcagataccgcccctcctttagaacacaagaaagggatcacactggagagaaacccaatgcttgtaaagtatgtggaaaaacctttatttcccattcaagtgttcgaagacacatggtaatgcacagtggggatggaccttataaatgtaagttttgtgggaaagccttccattgtctcagattatatcttatccatgaaagaattcacactggagagaaaccatgtgaatgtaaacagtgtggtaaatcctttagttattctgctacccatcgaatacataaaagaactcacactggagaaaagccttatgaatatcaggagtgtgggaaagcatttcatagtcccagatcctatcgtagacatgaaaggattcacatgggagaaaaggcttatcaatgtaaggaatgtggaaaagcattcacgtgtccccgttatgttcgtatacatgaaaggacccactctaggaaaaatctctatgaatgtaagcagtgtgggaaagcattatcctctcttacaagttttcaaacacacgtaagattgcactctggagaaagaccttatgaatgtaagatatgtggaaaagacttttgttctgtgaattcatttcaaagacatgaaaaaattcacagtggagagaaaccctataaatgtaagcagtgtggtaaagccttccctcattccagttcccttcgatatcatgaaaggactcacactggagagaaaccctatgagtgtaagcaatgtgggaaagccttcagatctgcctcacaccttcgagtgcatggtaggactcacactggagagaaaccgtatgaatgtaaggaatgtgggaaagccttcagatatgtgaataaccttcaaagtcatgaaaggacacaaacacacataagaatacactctggagaaagacgttataaatgtaagatatgtgggaaaggcttttattgtcccaaatcatttcaaagacatgaaaaaactcacactggagagaaactctatgaatgcaagcaacgttcagtagttccttcagtagttccagttccttttgatatcatgaaaggactcacactggagagaagccctataaatgcgagcaatgtgggaaagccttcagagctgtgtcaatcctttgaatgcatggtaggactcaccctgaagagaaaccctatgagtgtgagcaatgacggaaagccttcagatctgccccacacctttgaatacgtggtaggacacacaatggagagaaaccctatgcatgtaaggaatgtgggaaacccttcggatctgcccagaaccttcgaattcatgaaaggacacaaacacacataatgcactctgtagagagaccttataaatgtaagatatgtgggaggggcttttattctgccaagtcatttcaaatacatgaaaaatcttacactggagagaaaccctatgagtgtaagcaatgtgggaaagcctttgtttccttcacttcctttcgatatcatgaaaggactcacactggagagaacccctatgagtgtaagcaatttgggaaagccttcagatctgtcaaaaatcttcgatttcataaaaggacacacactggagagaaaccctgtgaatgtaagaaatgtagaaaagcattccataatttctcttctttgcaaatacatgaaaggatgcacagaggagagaagctctgtgaatgtaagcattgtgggaaagcattcatatctgccaagatcctttgaatacatgcaagaacacacaatggagagaaaccctatgaatgtaaagaatgcagaaaagcattcagcttgcctacttcctttcatagacatgaaaagactcacactggaaggaaacactatgaatgcaagcaatgtggcaaagctttcacttcttccagttcttttcaatatcatgaaagaacacactagggagaaaccctatcaatgtaagcattgtgcaaaagcctttatttcttccacttcttttcaatatcatgaaaggactcacatgggagagaaaccctatgagtgtatgccaagtgggaaagccttcatttcttctagttcccttcaatatcatgaaaggactcacactggagagaagccctatgaatataagcaatgtgggaaagccttcagatcagcctcgcaccttcaaatgcatggaaggactcacactggagaaaaaccctatgaatgtaagcagtatgggaaagcgttcagacctgacaagattctttgaatacagataatgaatgtaaacaattaactgtttataataactgtatactaacaaatgatattctttttaaataagaagctataatatcccattggtgtcatgtattagatcagccttatactgttaaattgttattatttggacattgtgagtcagtataaccatgtggataaaatgccagacatctttttattcgaaaattttacttttcatgcttctgtacttacatttttatctcaaccttaatttttctttctttttttttttcccccagaaagaatctcactctgtcacccaggctggagtgcagtggcgtgatctcaggtctactgcaacctctgcctccagggttcaagcaattctcctgcctcagtctcctgagtagctgggactacaggcatgcgccaccatgccaggctaattttttgtattttttagtagagacggggtttcaccatattcgctagctg

SEQ ID NO: 70 - DNA, 2095 bp, Homo sapiens
  misc_feature: Incyte ID No 7502244CB1
tgtgctggacaggcggcgttagtgggagcatttacatcatagaaatagcaaacactacatatctggacttcccccttctccctcaaagttggtttaccagcccagcaggagcatggcccagaaaagttgacctttcagcctggagtgaagaaaactggtggagtactgccaaagctccctgcagccacagtaattcgcttgagaaatggacctttaagccactgagttgcaggggttgttcgttacctgagcatcatctagccaaagctggtacaccatgctggtgttttggtgaacaccaataagcgccgccttcatctgtcccaacctaggttggagggatttcttccaataacttgtgacatcatgcgcctacagcctgcaaagatgcaaaccgcgctctgagactcaggcccgggaattcaggtcttggaaggttaatgcaaagttgtaaaacaatagttacatttcaaccacactcagctattgcgtcatcttaaaaccctggcaacacaaatctgtcgctcgcgatccttggcggtttcagagcgactcccctaccgctttcgggcgaaggccacacgtgacggcccccctttccccgcagacgtgtgaatgcagcgctgtgtctttaaggggcacgcgcggaggttttccgtccgggacaaaatgtcagcgaggcgcctggagggggatctaccatctcggactcccgacccgccgccggctccggccgcgtttcccgggtaaagggcactgctgatggttcttcagaactcaggaatcgtgttgcatgcattgtttctacccacctgagatggttggaaaccctgaggcaatgacagggaccccagaatccttgggaaattacccactgtctaatttaaagcgtgcgggcgccgcagaatgaggagtggcgagccggcctgcaccatggaccaggcccgcgggctggacgacgcggcggcgcggggcggtcagtgtccgggactggggccggcgccgacgccgacgcctcccggccgcctgggggcgccatactccgaggcctggggctacttccacctggcgccggggcgccccgggcatccgtcgggccactgggccacctgccgtctgtgcggggagcaggtgggccgcggcccgggcttccacgcggggacctcggcgttgtggaggcacctgaggagcgcgcaccggcgggagctggagagcagcggcgccgggagctccccacctgccgcgccctgcccgccgccgcccgtgcccgctgcgtgccccgagggcgactgggcgcgcctgctggaacagatgggcgcgctggccgtgcgcggcagcctggcgggagcgggagctgggagcggcgctgaggcggccgtggagcagggcgagcgcgccctggagcggaggcggagggcgctgcaggaggaagagcgcgccgcggcccaggcgcgccgggaactgcaggccgagcgggaggcgctgcaggcgcggctgcgggatgtgagccgccgtgagggcgccctgggctgggcccccgctgcgccgccgccgctcaaggacgaccccgagggtgacagggacggctgcgtcatcacaaaggtcctcctgtaggggtgtggccacttccccaccccaggacagcgcttctccgtccaatgccaatgccttcagaccccgctgggaccgaagccgtaaccgaagccaggcccgcagccgctactcactcggaagctccagctaactgtaggatcttccacaccctaaggcttcagcttgagaagcacttcgaagccagagcagaaccaaaactcacttccatggggtcaccgggggtgcctgggcggcctttcgtgggcatgcacgcaaggaattggggtggcacacgggacacccgagagctccaggagccccgtgaacccagaccacccagtgccatggccacttagggctgggaacccgaacctggtgactttgaaaggatagagcttcattccataccaaagacttatcacaccatgtgcctatacacccagcagcccaaggtggagggtgcgtggaaatgagaaagctttttacccaaaaaaaaaaaaaa

SEQ ID NO: 71 - DNA, 2109 bp, Homo sapiens
  misc_feature: Incyte ID No 7498718CB1
cttaaactgggcgtttgtattagttgggtttcccggtgtctctttagcaagtgaagtttctggttccctccttcactgtgtgacctgcctagtcctcctgggttgcgtttacagaagtttatacgagacctagtttccagggaagaactcactgatgccgcgagggagatggggtactggatgatggtcttcagccttaagggtacttcagtcttaaccgtgtgttataaggtttgaaagggagggttccctatgaataagaagcgcacttgaaagaacagccctctggtctaacctcccactggtgcttcagaggaggataaaaggtcacagctatgtttccagtgttctctggctgtttccaagagctacaagaaaagaataaatctctggagttggtgtcctttgaggaggtagctgtgcacttcacctgggaggagtggcaggacctggatgacgctcagaggaccctgtacagggacgtgatgctggagacctacagcagcctggtatcattggggcattgcattaccaaacctgagatgatcttcaagctagagcaaggagcagagccatggatagtagaagaaaccctaaacctgagactttcagctgtccagatcattgatgaccttattgaaaggagccatgaaagtcatgatagatttttctggcaaattgtaatcaccaacagcaacacatcaactcaggagagagttgaattaggaaaaacatttaatttgaactcaaaccatgttttaaatctgattataaataatggaaacagttcaggaatgaagcctgggcagtttaatgattgccagaacatgcttttccctattaagcctggggagacacagtctggagagaaacctcatgtctgtgatataaccaggagatcccacagacatcatgaacatcttactcagcatcacaagattcaaactctggtgcagacttttcaatgtaatgaacaagggaaaaccttcaacacggaggcaatgttctttatacataagagggttcatatagtacagacctttggtaaatataatgaatatgagaaagcctgtaataactcagctgttattgtccaagggataactcaggtaggacagccaacttgctgtagaaagtctgacttcactaaacatcagcagacacacacaggagagaaaccctatgaatgtgttgaatgtgagaaaccctccattagcaaatcagaccttatgctacagtgcaagatgcctactgaggaaaaaccttatgcctgtaactggtgtgaaaaattgttcagctataagtccagcctcattatccatcagagaattcacacaggggaaaagccctatggatgcaatgaatgtggaaaaacctttcgctgtaagtcattcctcactttacatgagagaactcacacaggggataaaccctacaaatgtattgaatgtggaaaaacttttcactgtaagtcacttctcactttacatcacagaactcactcaggggaaaagccctatcagtgtagtgaatgtggaaaaacctttagccagaagtcatacctcacaatacatcatagaactcacacaggggaaaagccctatgcatgtgaccattgtgaagaagcatttagccataagtcaaggcttactgtccatcagagaacacacacaggggaaaaaccgtatgaatgtaatgagtgtggaaaaccctttatcaataagtcgaacctcaggttacatcagagaactcacacaggggaaaaaccctatgaatgcaatgaatgtgggaaaacgtttcaccgtaagtcattcctcactatccatcaatggactcacacaggggaaaaaccctacgaatgcaatgaatgtgggaaaacctttcgctgtaagtcattcctcactgtccatcagagaactcatgctggggaaaaaccatacgcatgtaacgaatgtggaaaaacatatagccacaagtcataacctttacagtacattcacaggaactcacacaggggaaaaaccctatgaatgtaatgaatgtggaaaatcctttcactgtaagtcattcctaactatacatcagagaactcatgctggcaaaaaaccc

SEQ ID NO: 72 - DNA, 1440 bp, Homo sapiens
  misc_feature: Incyte ID No 6259308CB1
cggagcgtgcggggagggaggggcgcgtaggctccgcctccaacggccgccgccccaccccctgcgccctcgcaccttcgccaacctaatcacgcaccgccctacccgcccttccgttggcagcgccggcgtccgcgcgggaaggtataaaaacgaccacagtcgcggcccgactccctcaacagcgcccgccgagtctcgcacgccagtgcgcacgcgcctccccgcctcaccccgtcccgcgcgcgcagccgtccgccagcggccaatcagcagccgctccgaggccgtggcaccggaaggcctcacgcggcgccggaagtgacgtgccggcgtgctgacgcgcgggctcgagccgatgcccgattccgcgcccgccatggccgacaaaatggacatgtctctggacgacatcattaaactgaaccggagccagcgaggcggccggggcgggggccggggccgcggccgggccggctcccagggcggccgcggcggtggggcgcaggccgccgcgcgagtgaatcgaggcggcgggcccatccggaaccggccggccatcgcccgcggcgcggccggcggaggcggcaggaaccgaccggcgccctacagcaggccaaaacaacttcccgacaagtggcagcacgatcttttcgacagtggcttcggcggtggtgccggcgtggagacaggtgggaaactgctggtgtccaatctggattttggagtctcagacgccgatattcaggaactctttgctgaatttggaacgctgaagaaggcggctgtgcactatgatcgctctggtcgcagcttaggaacagcagacgtgcactttgagcggaaggcagatgccctgaaggccatgaagcagtacaacggcgtccctctggatggccgccccatgaacattcagcttgtcacgtcacagattgacgcacagcggaggcctgcacagagcgtaaacagaggtggcatgactagaaaccgtggcgctggaggttttggtggtggtggaggcacccggagaggcacccgcggaggcgcccgtggaagaggcagaggtgccggcaggaattcaaagcagcagctttcggcagaggagctggatgcccagctggacgcctataatgcgagaatggacaccagttaaacagaccagcaaatccgcgtgcggaacaggacccaggcgtctcctcttgctccctggttggggggcggtggctggggctgtgcggccaatgatggatttgtttcttttatgttttaaaataggatttaaaaactcatgtaaaggttttttttttttctttttttttttttttaattctgaaacagacctgttttgtaccgagttatttttgggataaattttactggttgctgttgtggagaaggtggcgtttccaccttttccataataaaatagaaatgtgtgta

SEQ ID NO: 73 - DNA, 2794 bp, Homo sapiens
  misc_feature: Incyte ID No 7504104CB1
gaccattctcgctacagaggttgacttcctcggggtccagagagggtcacccctaagcctctgtcagtaagaacactggcgtgttctctcagtcacacaccagagagaatggagctaacacataagtagcagctaaattctgagtttctgagcatacgcagagatggagttgtgcttggggccagaaaggaggtctcctgtgtggtgacaaacatctctttccattttcttttgctcagtccatttgcacaggttcgagggagttgatggagggtcagacgagaggtggtgggagctcaggattttctattcccacaaagagctaagacttaggagtgctggccccagagatggtgcggccctctttctacttctgtttctgtgggcnnnnnnnnnnnnnnnnnnnnnnnnnnnnnnnnnnnnnnnnnnnnnnnnagcaggactgagtaccagttaaaatactttttggggatacacatgtgagatactaagtacttgcagaagatttttgtctctctttttaaagtctctttccttggaatattgtgagaatatttgtggccatttaaggtttgtgtgattttgctaaaatgcatcaccaacagcgaatggctgccttagggacggacaaagagctgagtgatttactggatttcagtgcgatgttttcacctcctgtgagcagtgggaaaaatggaccaacttctttggcaagtggacattttactggctcaaatgtagaagacagaagtagctcagggtcctgggggaatggaggacatccaagcccgtccaggaactatggagatgggactccctatgaccacatgaccagcagggaccttgggtcacatgacaatctctctccaccttttgtcaattccagaatacaaagtaaaacagaaaggggctcatactcatcttatgggagagaatcaaacttacagggttgccaccaggtctatgctccatcagcaagcactgccgactacaatagggactcgccaggctatccttcctccaaaccagcaaccagcactttccctagctccttcttcatgcaagatggccatcacagcagtgacccttggagctcctccagtgggatgaatcagcctggctatgcaggaatgttgggcaactcttctcatattccacagtccagcagctactgtagcctgcatccacatgaacgtttgagctatccatcacactcctcagcagacatcaattccagtcttcctccgatgtccactttccatcgtagtggtacaaaccattacagcacctcttcctgtacgcctcctgccaacgggacagacagtataatggcaaatagaggaagcggggcagccggcagctcccagactggagatgctctggggaaagcacttgcttcgatctattctccagatcacactaacaacagcttttcatcaaacccttcaactcctgttggctctcctccatctctctcagcaggcacagctgtttggtctagaaatggaggacaggcctcatcgtctcctaattatgaaggacccttacactctttgcaaagccgaattgaagatcgtttagaaagactggatgatgctattcatgttctccggaaccatgcagtgggcccatccacagctatgcctggtggtcatggggacatgcatggaatcattggaccttctcataatggagccatgggtggtctgggctcagggtatggaaccggccttctttcagccaacagacattcactcatggtggggacccatcgtgaagatggcgtggccctgagaggcagccattctcttctgccaaaccaggttccggttccacagcttcctgtccagtctgcgacttcccctgacctgaacccaccccaggacccttacagaggcatgccaccaggactacaggggcagagtgtctcctctggcagctctgagatcaaatccgatgacgagggtgatgagaacctgcaagacacgaaatcttcggaggacaagaaattagatgacgacaagaaggatatcaaatcaattactaggtcaagatctagcaataatgacgatgaggacctgacaccagagcagaaggcagagcgtgagaaggagcggaggatggccaacaatgcccgagagcgtctgcgggtccgtgacatcaacgaggctttcaaagagctcggccgcatggtgcagctccacctcaagagtgacaagccccagaccaagctcctgatcctccaccaggcggtggccgtcatcctcagtctggagcagcaagtccgagaaaggaatctgaatccgaaagctgcgtgtctgaaaagaagggaggaagagaaggtgtcctcggagcctccccctctctccttggccggcccacaccctggaatgggagacgcatcgaatcacatgggacagatgtaaaagggtccaagttgccacattgcttcattaaaacaagagaccacttccttaacagctgtattatcttaaacccacataaacacttctccttaacccccatttttgtaatataagacaagtctgagtagttatgaatcgcagacgcaagaggtttcagcattcccaattatcaaaaaacagaaaaacaaaaaaaagaaagaaaaaagtgcaacttgagggacgactttctttaacatatcattcagaatgtgcaaagcagtatgtacaggctgagacacagcccagagactgaacggcaatctttccacactgtggaacaatgcatttgtgcctaaacttcttttggaaaaaaaaaaaaaaaaaaagat

SEQ ID NO: 74 - DNA, 2505 bp, Homo sapiens
  misc_feature: Incyte ID No 7504121CB1
cgggattcgggcgccgcggcagctgctccggctgccggccggcggccccgcgctcgcccgccccgcttccgcccgctgtcctgctgcacgaacccttccaactctcctttcctcccccacccttgagttacccctctgtctttcctgctgttgcgcgggtgctcccacagcggagcggagattacagagccgccgggatgccccaactctccggaggaggtggcggcggcgggggggacccggaactctgcgccacggacgagatgatccccttcaaggacgagggcgatcctcagaaggaaaagatcttcgccgagatcagtcatcccgaagaggaaggcgatttagctgacatcaagtcttccttggtgaacgagtctgaaatcatcccggccagcaacggacacgaggtggccagacaagcacaaacctctcaggagccctaccacgacaaggccagagaacaccccgatgacggaaagcatccagatggaggcctctacaacaagggaccctcctactcgagttattccgggtacataatgatgccaaatatgaataacgacccatacatgtcaaatggatctctttctccacccatcccgagaacatcaaataaagtgcccgtggtgcagccatcccatgcggtccatcctctcacccccctcatcacttacagtgacgagcacttttctccaggatcacacccgtcacacatcccatcagatgtcaactccaaacaaggcatgtccagacatcctccagctcctgatatccctactttttatcccttgtctccgggtggtgttggacagatcaccccacctcttggctggttttcccatcatatgattcccggtcctcctggtccccacacaactggcatccctcatccagctattgtaacacctcaggtcaaacaggaacatccccacactgacagtgacctaatgcacgtgaagcctcagcatgaacagagaaaggagcaggagccaaaaagacctcacattaagaagcctctgaatgcttttatgttatacatgaaagaaatgagagcgaatgtcgttgctgagtgtactctaaaagaaagtgcagctatcaaccagattcttggcagaaggtggcatgccctctcccgtgaagagcaggctaaatattatgaattagcacggaaagaaagacagctacatatgcagctttatccaggctggtctgcaagagacaattatggtaagaaaaagaagaggaagagagagaaactacaggaatctgcatcaggtggaaaacgaagctcattcccaacgtgcaaagccaaggcagcgaccccaggacctcttctggagatggaagcttgttgaaaacccagactgtctccacggcctgcccagtcgaccccaaaggaacactgacatcaattttaccctgaggtcactgctagagacgctgatccataaagacaatcactgccaacccctctttcgtctactgcaagagccaagttccaaaataaagcataaaaaggttttttaaaaggaaatgtaaaagcacatgagaatgctagcaggctgtggggcagctgagcagcttttctccccccatatctgcgtgcacttcccagagcatcttgcatccaaacctgtaacctttcggcaaggacggtaacttggctgcatttgcctgtcatgcgcaactggagccagcaaccagcacatccatcagcaccccagtggaggagttcatggaagagttccctctttgtttctgcttcatttttctttcttttcttttctcctaaagcttttatttaacagtgcaaaaggatcgttttttttttgcttttttaaacttgaatttttttaatttacactttttagttttaattttcttgtatattttgctagctatgagcttttaaataaaattgaaagttctggaaaagtttgaaataatgacataaaaagaagccttctttttctgagacagcttgtctggtaagtggcttctctgtgaattgcctgtaacacatagtggcttctccgcccttgtaaggtgttcagtagagctaaataaatgtaatagccaaacccactctgttggtagcaattggcagccctatttcagtttattttttcttctgttttcttcttttctttttttaaacagtaaaccttaacagatgcgttcagcagactggtttgcagtgaattttcatttctttccttatcacccccttgttgtaaaaagcccagcacttgaattgttattactttaaatgttctgtatttgtatctgtttttattagccaattagtgggattttatgccagttgttaaaatgagcattgatgtacccattttttaaaaaagcaaggcacagcctttgcccaaaactgtcatcctaacgtttgtcattccagtttgagttaatgtgctgagcatttttttaaaagaagctttgtaataaaacatttttaaaaattgtcaaaaaaaaaaaaaaaaagatcgg

SEQ ID NO: 75 - DNA, 4115 bp, Homo sapiens
  misc_feature: Incyte ID No 5635695CB1
cctgtaaggcggggagacaatgagtaaactctccttccgagcgcgggcgctggacgccgccaagccgctgcctatctaccgcggcaaggacatgcctgatctcaacgactgcgtctccatcaaccgggccgtgccccagatgcccaccgggatggagaaggaggaggaatcggaacatcatttacagcgagcaatttcagcacagcaagtgtttagagaaaaaaaagagagtatggtcattcctgttcctgaggcagagagcaacgtcaactattacaatcgcttgtacaaaggagagtttaaacagccaaaacagttcattcatattcagccttttaatctagacaacgagcaaccagattatgatatggattcagaagatgagactttattaaatagacttaacagaaagatggaaattaagcctttgcaatttgaaattatgattgacagacttgaaaaagccagttctaatcagcttgtaacacttcaagaagcaaaactgctgctaaacgaagatgattaccttattaaagctgtatatgactactgggtgagaaaacgtaaaaactgcagggggccatccctcattcctcagataaaacaagagaaaagagatggctctaccaacaatgacccttatgttgcctttcggagaagaacagagaaaatgcaaactcgaaagaatcgtaagaatgatgaagcctcttatgaaaagatgttgaaactgagacgagaatttagtagagccataacaattttggaaatgattaagagaagagagaaaacaaaacgagaattattgcacttaaccttagaagttgtggagaaaagataccatttgggagactatggtggtgaaatccttaatgaagtaaaaatcagtagatcagaaaaagagttatatgccactccagcaactcttcataatggaaatcatcacaaagttcaagaatgtaaaactaagcaccctcatcatttgtctttgaaagaagaggcttctgatgtggttcgtcaaaagaagaagtacccaaagaagcctaaagcagaggctttgataacatctcagcaacccactcctgagacattgcctgtgatcaataagagtgacattaagcaatatgattttcacagctcagatgaagatgaatttccacaggtattgtccccagtatcagaaccggaagaagaaaatgatcctgatggtccctgtgctttcagaaggcgggcaggatgccagtattatgctcctcgtttggaccaagctaaccattcatgtgaaaattcagaattggcagatttggataagttgaggtataggcattgccttacaacacttacagtcccaagaagatgtataggatttgcaaggaggcgaattggcagaggtggaagggtcataatggaccgaatatccacagaacatgacccagtcctgaaacagatagaccctgaaatgctgaatagtttttcaagctcttcccaaactatagacttttcttctaatttctctcggaccaatgcttccagtaaacattgtgaaaatagactgtctctttctgaaatattaagcaatatcagatcatgtcgactacagtgtttccagccaaggctactaaatttacaggacagtgatagtgaagaatgtacctcaagaaaaccagggcagactgtgaacaataaaagagtttctgcagcatctgtagctttattgaacaccagcaagaatggcatatcagtaacagggggtatcacagaagagcagtttcagacacatcagcagcagttagttcagatgcaaaggcagcaacttgcccagcttcagcagaaacagcaatctcagcattcctcgcaacagacacatccaaaagcacagggctcaagcacctctgactgtatgtctaaaacacttgactcagccagcgcccactttgctgcatctgcagtggtcagtgcacctgttccaagtcgcagtgaggtagccaaggaacagaacactggccacaacaacataaacggtgttgtccagccttcaggaacctctaaaacattatactccaccaatatggctttatcatccagcccagggatttcagctgtacagcttgtaaggacagttggccacaccactacaaaccacttaatcccagcattgtgcacaagcagtcctcagacacttcccatgaacaattcctgcctgacaaatgcagtgcacctcaataatgtcagtgttgtttctccagtcaatgtgcatatcaatacacggacttcagcaccatcgccaacagccttaaaacttgccacagttgctgccagtatggacagagtgccaaaggttactcccagcagtgccatcagcagcatagcaagagagaaccacgaaccagaaagattgggcttaaatggaatagcagagacaacagtagctatggaagtgacataacctaaaacacgtggctctgacctgtgctgatggtgtgcagtcattcatattccagctgaatgcaaaaggcaacactctgtggatcacagagtgtaacaatggacctaaatggactatagtatattggatgttaaatccatatatgatgtatattttgtaaaattgggaaaatcactaccttgtaaaatagtttatttgtatcatcaatattatttctgttacttgaatagtagatattcatcatcatgcttttgcacttgaatttgcaactgaatggattttaaaaaataattctttaatgggatcatgagcatgaaatgggatcctgcatcacttgttttaactatttattttgccatgtttacattttgtatcttgtaaaaataaatccaactttgtgtctaaaaagttaaagattcatagctaggaaatgaaattcttgtaatttttttctaaaggaactgtaaagttttcacttggttcattttgtttcacaatttgactagatggactttttggtaaatactttagtggcatttcactgtcaaatatgaagttcaaggcaaaatagtattttctattactgtgcaggggaaagggatggatcgatacatgcaaatttaatgtagtaactcacttttccatatattttgaatgtatatttctatttatgataccaatttataaaaaataattacacagaaaaaaatggaataggaaaaattatgcatctagcacatttaaactgtgcaaatatgaaaatttttcgaggattacattttatctgaaggctgcatattttaactggctttaaaactgtaacacatcacataaaagatactttaccaggtatgtattgcattatatcattgcaataattattggaagtctagatatcgagccatcccaggtgttgggcggggggagggttgtggcaagattgtcttttcaattttggagagttttcctgtggctacaaggcaagtaacgggttggaaaaagtctgactgtaagcgttggacaccttcatagtgtagtgttttagtgactttttttatacggttcttgtaaattagatacgtgtagtggtgtttcagaatgtttgtttatgcactagttcagacaactttccctgttacttgttcttgataagtgaaaactgcagggaaataaaaaatacatatcaaaacatggacatgctgcatatgtgtttatttcacaatgtgcacacagtataagtgaaaatttaagggagatgatagcacttaacagcacttttcatgttcacatgctttccaagcattaatgaaaagaactataggaagctcatctgtgggctctattgggtttcagataatccaatataaactacctttgatatgaaagttcgtaagatattttacagaatgtaagtaatttgcagtatcgcagtcattgaaatgtcataagtgagcctcattttataaataaaagtttagagtagaattatattgcaagggggttttgtcaagtaagtactgggacaatgtaaatatttatgtaagtgaatacgaattaaaaccatgttcaaacttacataattttatacctttacattttttatatctgcagtcctgaaagattgtaaattgaaatgtcagttgaattaatgggccagatttctgaatgaaag

SEQ ID NO: 76 - DNA, 1027 bp, Homo sapiens
  misc_feature: Incyte ID No 7503983CB1
ggcggccaggcggggcgtgtgcaagggtcgcgtcccccccccgggcccccggcccgtggctcttggtagagcccagtgcttcatttcccgtgcgcggcccgggcggccctccctttcatcagtcttcccgcgtccgccgattcctcctccttggtcgccgcgtccttggctggcgtcagaaaaatggctacaaacttcctagcacatgagaagatctggttcgacaagttcaaatatgacgacgcagaaaggagattctacgagcagatgaacgggcctgtggcaggtgcctcccgccagagctcaggccccggggcctccagcggcaccagcggagaccacggtgagctcgtcgtccggattgccagtctggaagtggagaaccagagtctgcgtggcgtggtacaggagctgcagcaggccatctccaagctggaggcccggctgaacgtgctggagaagagctcgcctggccaccgggccacggccccacagacccagcacgtatctcccatgcgccaagtggagcccccagccaagaagccagccacaccagcagaggatgacgaggatgatgacattgacctgtttggcagtgacaatgaggaggaggacaaggaggcggcacagctgcgggaggagcggctacggcagtacgcggagaagaaggccaagaagcctgcactggtggccaagtcctccatcctgctggatgtcaagccttgggatgatgagacggacatggcccagctggaggcctgtgtgcgctctatccagctggacgggctggtctggggggcttccaagctggtgcccgtgggctacggtatccggaagctacagattcagtgtgtggtggaggacgacaaggtggggacagacttgctggaggaggagatcaccaagtttgaggagcacgtgcagagtgtcgatatcgcagctttcaacaagatctgaagcctgagtgtgtgtacgtgcgcgcgtgcgtgaggccctgccacgattaaagactgagaccggccaaaaa

SEQ ID NO: 77 - DNA, 1103 bp, Homo sapiens
  misc_feature: Incyte ID No 7503476CB1
gcggacccctcaaagaactttgccctgaaccaaaaattcggccgcggaatccaagacaaacaaataagaaattacgaagatgccggaccggctcaaaaaaaaaacctcgcctgaagagcttcagacaacgaggcccttagggtcgggctttaggcggttccctgaccagggcgccagaaaagggcttggctcaagcaggcacgggggcgtgcagtacagcacacctagccccgattcttcaacagttctcgccctccgagcctagcacaacgagcctcaccgaaaccgtacaccgccaccaggacttccgtgatgggggatcaccaccctcagaaagaggaagcgactagcaggcgcgcaatcccgcgagaccaggaggccccgcccgaagcccggcctctgtgaccggaagtgaggcgttttgccccgcccccgtggccgatacctcgcgagacttggcgaaggccttcctttttcgtctgggctgccaacatgccatccagactgaggaagacccggaaacttaggggccacgtgagccacggccacggccgcataggcaagcaccggaagcaccccggcggccgcggtaatgctggtggtctgcatcaccaccggatcaacttcgacaaatatgaacagacacgggtgaatgctgctaaaaacaagactggggctgctcccatcattgatgtggtgcgatcgggctactacaaagttctgggaaagggaaagctcccaaagcagcctgtcatcgtgaaggccaaattcttcagcagaagagctgaggagaagattaagagtgttgggggggcctgtgtcctggtggcttgaagccacatggagggagtttcattaaatgctaactacttttaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagaaaaaaaaaaaagaaaaaaaaaaaaaaaaaaaagaaaaaggaaaaaaaagaagaggggggcccctttagggggccccaaattagggaggggaacatgggggggaaaagggtttataaaggaacccccaga

SEQ ID NO: 78 - DNA, 822 bp, Homo sapiens
  misc_feature: Incyte ID No 7504023CB1
cgccaggcgtcctcgtggaaggcccgggaccgcgggatgggtgtcggcgtgaccaggcctgagctccctgtctctcctcagtgacatcgtctttaaaccctgcgtggcaatccctgacgcaccgccgtgatgcccagggaagacagggcgacctggaagtccaactacttccttaagatcatccaactattggatgattatccgaaatgtttcattgtgggagcagacaatgtgggctttgtgttcaccaaggaggacctcactgagatcagggacatgttgctggccaataaggtgccagctgctgcccgtgctggtgccattgccccatgtgaagtcactgtgccagcccagaacactggtctcgggcccgagaagacctcctttttccaggctttaggtatcaccactaaaatctccaggggcaccattgaaatcctgggtgtccgcaatgttgccagtgtctgtctgcagattggctacccaactgttgcatcagtaccccattctatcatcaacgggtacaaacgagtcctggccttgtctgtggagacggattacaccttcccacttgctgaaaaggtcaaggccttcttggctgatccatctgcctttgtggctgctgcccctgtggctgctgccaccacagctgctcctgctgctgctgcagccccagctaaggttgaagccaaggaagagtcggaggagtcggacgaggatatgggatttggtctctttgactaatcaccaaaaagcaaccaacttagccagttttatttgcaaaacaaggaaataaaggcttacttcttaaaaaaaaaaaaa

SEQ ID NO: 79 - DNA, 877 bp, Homo sapiens
  misc_feature: Incyte ID No 7504128CB1
gaggccaagaattcggcacgagggctcggaggaggccaaggtgcaacttccttcggtcgtcccgaatccgggttcatccgacaccagccgcctccaccatgccgccgaagttcgaccccaacgagatcaaagtcgtatacctgaggtgcaccggaggtgaagtcggtgccacttctgccctggcccccaagatcggccccctgaggattacagtgaaactgaccattcagaacagacaggcccagattgaggtggtgccttctgcctctgccctgatcatcaaagccctcaaggaaccaccaagagacagaaagaaacagaaaaacattaaacacagtgggaatatcacttttgatgagattgtcaacattgctcgacagatgcggcaccgatccttagccagagaactctctggaaccattaaagagatcctggggactgcccagtcagtgggctgtaatgttgatggccgccatcctcatgacatcatcgatgacatcaacagtggtgctgtggaatgcccagccagttaagcacaaaggaaaacatttcaataaaggatcatttgacaactggaaaaaaaaaaaaaaaaaaaaaaaaaaaaaaaaaaaaaaaaaaaaaaaaaaaaaaaaaaaaaaaaaaaaaaaaaaaacaaaaaaaaaaaaaaaaaaaaaaaaaaaacacccccccctctaagatacactcaccaccatttctttctcgtctactacttatgaacaacatacctttcttttattaatatgctgccacatatattatattatcatcggcgccgcttttataaaaaacatgttctgaaaaaaacacctctgtccacaacaattaacattgggccctcccctcatcaaaaactggt

SEQ ID NO: 80 - DNA, 1306 bp, Homo sapiens
  misc_feature: Incyte ID No 4529338CB1
agatctgaatttaacaaatgcttagtctcagcagcctccgccggaattccggccggaattccgggagctgcggagcctggaatatggtcggggaaatggaaacgaaggagaagccgaagcccaccccagattacctgatgcagctgatgaacgacaagaagctcatgagcagcctgcccaacttctgcgggatcttcaaccacctcgagcggctgctggacgaagaaattagcagagtacggaaagacatgtacaatgacacattaaatggcagtacagagaaaaggagtgcagaattgcctgatgctgtgggacctattgttcagttacaagagaaactttatgtgcctgtaaaagaatacccagattttaattttgttgggagaatccttggacctagaggacttacagccaaacaacttgaagcagaaaccggatgtaaaatcatggtccgaggcaaaggctcaatgagggataaaaaaaaggaggagcaaaatagaggcaagcccaattgggagcatctaaatgaagatttacatgtactaatcactgtggaagatgctcagaacagagcagaaatcaaattgaagagagcagttgaagaagtgaagaaattattggtacctgcagcagaaggagaagacagcctgaagaagatgcagctgatggagcttgcgattctgaatggcacctacagagatgccaacattaaatcaccaacagcccaggctgctccaaggatcattactgggcctgcgccggttctcccaccagctgccctgcgtactcctacgccagctggccctaccataatgcctttgatcagacaaatacagaccgctgtcatgccaaacggaactcctcacccaactgctgcaatagttcctccagggcccgaagctggtttaatctatacaccctatgagtacccctacacattggcaccagctacatcaatccttgagtatcctattgaacctagtggtgtattaggtgcggtggctactaaagttcgaaggcacgatatgcgtgtccatccttaccaaaggattgtgaccgcagaccgagccgccaccggcaactaacctatgaccttctgacctctgaactcttcacccaatgatgacctgaccatgcctgcctgctgatcagttaactggtaatcgcctttgcttgcctgtcgtcagtgcagcgagctgaggcacttgtccgttcgtcttaccatctaaccaacaaaagacaaagaaattgttgtcctccaactcagttctttcagccagggggagaaatcagatctgcgggcccc

SEQ ID NO: 81 - DNA, 1016 bp, Homo sapiens
  misc_feature: Incyte ID No 7503460CB1
aactcgctcggccgccgccatcttgcgagctcgtcgtactgaccgagcggggaggctgtcttgaggcggcaccgctcaccgacaccgaggcggactggcagccctgagcgtcgcagtcatgccggccggacccgtgcaggcggtgcccccgccgccgcccgtgcccacggagcccaaacagcccacagaagaagaagcatcttcaaaggaggattctgcaccttctaagccagttgtggggattatttaccctcctccagaggtcagaaatattgttgacaagactgccagctttgtggccagaaacgggcctgaatttgaagctaggatccgacagaacgagatcaacaaccccaagttcaactttctgaaccccaatgacccttaccatgcctactaccgccacaaggtcagcgagttcaaggaagggaaggctcaggagccgtccgccgccatccccaaggtcatgcagcagcagcagcagaccacccagcagcagctgccctcatactttttatgtattagaatcatattcgtattgcccttttaaaacattgggatcctccaaaggcctgccccatgtatttaacagtaatacaggaagcatggcaggcaccatgcaaaccaaggatggatggtgcagtccctgtgtcagtgggcggtggtttcctgctggcctggaatcactcatcacctgattgattggctctgtggtcctgggcaggtgcctcataggtgtgtggatatgatgacgtttctttaaaatgtatgtatttaacaaatacttaattgtattaaggtcatgtaccaaggatttgataaagtttaaataatttactctctacttttatcccttttaatccttttaactcatgttattcctcatgtgagtaatacctgtttaacacttgaggtaaactgagggcacaggggaccctaggtttgcaatgcctaggtcaacacatttggaaaaggtgaaagagggatttgttgcaaaaaccggacaaatccggggg

SEQ ID NO: 82 - DNA, 4204 bp, Homo sapiens
  misc_feature: Incyte ID No 5466630CB1
cacggccggagttggtggtctgggaacccacgtgggctgggtttcggattgctctgctggtccggccgctggagcgcccaccctggcctagtcgccatggggaagctgcgccggcgctacaacatcaaggggcgccagcaggcgggcccccggaccctcgaagggcccccccgagccgccccccgtgcagctggaactggaggacaaggacacgttgaagggagttgatgcaagcaacgcgctcgttctaccggggaagaagaaaaagaagaccaaagcccctcccctgtcgaagaaggagaagaagcctctgaccaagaaggagaagaaagtgctgcagaaaatcttagaacagaaggagaaaaagagccagcgagcagagatactacagaagctgagtgaagtccaggcttccgaagctgagatgagactcttttataccacttccaagctaggcactgggaaccgcatgtatcacaccaaagagaaggctgacgaggtggtagccccgggccaggagaagatcagtagcctcagcggtgcccaccggaagcgtcgccgctggcctcagctgaggaggaggacggaggaggaggaggagtcggaatcggagctggaggaggagtcggagctggacgaggacccagctgctgagccggctgaggctggtgtggggaccaccgtggcacctctgccgccagctccagcacccagcagtcagcccgtgccggctgggatgactgttcctcctcctccagctgcagccccaccactgcccagggccctggctaagcccgccgtcttcatccccgtgaaccgctccccggaaatgcaggaggaacggctgaagctcccaattctctccgaagaacaagtaatcatggaggctgtggccgagcaccccatcgtcatcgtgtgtggtgagaccggcagcgggaagaccacacaggtgcctcagtttctctatgaagcaggcttcagcagtgaagacagcatcatcggtgtcacggagccccgccgagtggccgccgtggccatgtcccagcgagtggccaaggagatgaatctgtcccagcgggtcgtctcctaccagatccggtatgaaggaaacgtgacagaggagaccagaatcaagttcatgacggatggtgtgctgcttaaagaaatccagaaggacttcctgctgctgcggtacaaggtggtgatcatcgacgaggcccacgagaggagcgtgtacacggacatcctcatcggcctcctgtcccgcattgtgactctccgggctaagaggaacctgccactcaagctgctcatcatgtcggccacgctgcgggtggaggacttcacccagaacccacggctcttcgccaagccgccgccggtcatcaaggtggaatccaggcagttcccagtgactgtgcatttcaacaagcggacaccgctggaagactacagtggcgagtgcttccggaaggtctgcaagatccaccggatgctgcccgcaggtggcatcctggtgttcctgacggggcaggctgaggtgcatgcgctgtgccgcaggctcaggaaggctttcccaccctccagagcccggccacaagaaaaggacgacgatcagaaagactcggtggaggaaatgcggaagtttaagaagtcaagggccagggccaagaaggcgcgggctgaggtgctgccccagatcaacttggatcattactcggtgttaccggcaggcgaaggcgatgaggacagggaggcagaagtggatgaggaagagggggccctggactccgacctcgatctggacctgggggatggcgggcaagatggaggtgagcagccggatgcctccctcccgctccacgtgctcccgctgtactctctgctggccccagagaagcaagcacaggtctttaagcctccaccggaggggactcggttgtgtgttgtggccaccaatgtggccgagacgtcgcttaccatccctggcatcaagtacgtggtggactgtgggaaggtcaagaaacgctactacgaccgcgtcactggcgtatcctccttccgtgtcacctgggtctcccaggcatcagctgaccagcgagcgggcagagcaggacggacggagcccggccactgctacaggctgtattcatctgcggtttttggtgacttcgagcagtttcctcctccagaaatcacccggaggcctgttgaagacttaatccttcaaatgaaggcgctcaacgttgaaaaggtcatcaacttccccttcccgacgcccccctccgtggaagcccttcttgccgccgaggagctgttgatcgcactgggtgccctgcaaccgccccagaaagcagaaagggtgaagcaactgcaggagaaccggctgagctgccccatcactgcgctgggccggacaatggccacgttccccgtggcaccccgctacgctaagatgctggcactgagccgacaacacggctgcctgccctatgccatcaccatcgtggccagcatgacggtgcgggagctgtttgaggagctggacagaccagcggccagtgacgaggagctcaccaggctgaagagcaagcgggcccgggtggcccagatgaagaggacctgggcagggcagggggcttctctgaagctcggcgacctcatggtgctgctgggcgccgtgggagcctgtgaatatgccggctgcacaccccagttttgcgaagccaacgggctgcggtacaaagccatgatggagatccggcgcctgcggggccagctgaccaccgcagtcaatgccgtgtgccccgaggctgagctcttcgtggatcccaagatgcagccgcccaccgagagccaggtgacctacctgcgacagatcgtgacggcgggcctgggggaccacttggcccgcagggtccagagcgaggagatgctggaggacaagtggaggaacgcctacaagacccctctcctcgacgaccctgtcttcatccaccccagctccgtccttttcaaagagctccccgagtttgtggtctaccaggaaatcgtggagaccactaagatgtacatgaaaggcgtctctagcgtggaggtccagtggatcccggccctgctgccctcttactgccagtttgacaagcccctggaggaaccagcccctacatactgccccgagcgggggcgggtgctgtgtcaccgggccagcgtgttctatcgcgtgggctggccgctccccgccatcgaggtggattttccagaggggattgaccgctacaagcactttgctcggttcctgctggaagggcaggtcttccgcaagctggcctcataccagagctgtctgctgtccagccccggcaccatgctgaagacgtgggccaggctgcagccccgtacggagagccttctgcgagccctggttgcagagaaggctgactgccatgaagccttgctggctgcttggaagaaaaaccccaaatacctgctggctgagtactgtgagtggcttccacaggccatgcaccccgatatcgagaaagcctggccccccaccactgtccactgaccagaaacctggctgcagggccgaggactggtttggggactggagggctggcagcagcctgtcaccgtgcgaccgtgaccacctggcatgggcttcgtggcctgctctcaggaagtgggtcaagccctgggaaccctcatccatgagagctcgatcccgtatgaagggtgctgccgcccgtgccatctggcccgggggtgactttttgaactgtttattatatggtggatgatgatttcatctcacgtgctggacgctgttctgttcagtgtgctctttggactacattagtcccctgtggagcagcagggctggagatctctgcagtcccttccccgcccgccctgccagaaggccgaggaggcacgtggagggcctccttcctgcaattcttccctctccagagtcagggagggctgcccagccctggcctcacagccgtcccagatgttaggtgagccactgagctctgtgttgaccttgaggggcctggctgggggcccccaggctccatgccttcttgggagggtggcgcaacgcctttctgtgttatggcaacagggagtgggcatctcatctgctgtggtcagtctcagacggagggagggagctgacgtggtgtgttggtcaacg

SEQ ID NO: 83 - DNA, 2211 bp, Homo sapiens
  misc_feature: Incyte ID No 7503474CB1
ccgtggggcagtcgaggatgtcggtgaattacgcggcggggctgtcgccgtacgcggacaagggcaagtgcggcctcccggagatcttcgaccccccggaggagctggagcggaaggtgtgggaactggcgaggctggtctggcagtcttccaatgtggtgttccacacgggtgccggcatcagcactgcctctggcatccccgacttcagggacaaactggcagagctccacgggaacatgtttgtggaagaatgtgccaagtgtaagacgcagtacgtccgagacacagtcgtgggcaccatgggcctgaaggccacgggccggctctgcaccgtggctaaggcaagggggctgcgagcctgcaggggagagctgagggacaccatcctagactgggaggactccctgcccgaccgggacctggcactcgccgatgaggccagcaggaacgccgacctgtccatcacgctgggtacatcgctgcagatccggcccagcgggaacctgccgctggctaccaagcgccggggaggccgcctggtcatcgtcaacctgcagcccaccaagcacgaccgccatgctgacctccgcatccatggctacgttgacgaggtcatgacccggctcatgaagcacctggggctggagatccccgcctgggacggcccccgtgtgctggagagggcgctgccacccctgccccgcccgcccacccccaagctggagcccaaggaggaatctcccacccggatcaacggctctatccccgccggccccaagcaggagccctgcgcccagcacaacggctcagagcccgccagccccaaacgggagcggcccaccagccctgccccccacagaccccccaaaagggtgaaggccaaggcggtccccagctgaccagggtgcttggggagggtggggctttttgtagaaactgtggattctttttctctcgtggtctcactttgttacttgtttctgtccccgggagcctcagggctctgagagctgtgctccaggccaggggttacacctgccctccgtggtccctccctgggctccaggggcctctggtgcggttccgggaagaagccacaccccagaggtgacagctgagcccctgccacaccccagcctctgacttgctgtgttgtccagaggtgaggctgggccctccctggtctccagcttaaacaggagtgaactccctctgtccccagggcctcccttctgggccccctacagcccaccctacccctcctccatgggccctgcaggaggggagacccaccttgaagtgggggatcagtagaggcttgcactgcctttggggctggagggagacgtgggtccaccaggcttctggaaaagtcctcaatgcaataaaaacaatttctttcttgcaaaaaaaaaaaaaaaaaaaaaaaaaaaaaaaaaaaaaaaaaaaaaaaaaaaaaaaaaaaaaaaaaaaagggggggcgcaccagcagatgaagacggaagcagacaacacccgagcgaataacatacatcacaggaacggcaaacaaagagggagcatataaccacaagcaaaagagaagaagaaaaaaaacaagaagaagacggagcgcacaataaatgagagggagacctacaatatataataaaagagaaaagaagagggggggtaaaccgggagttcaataaggaaaatagcgagaaaaagtgagaccaagagaggaatagaaaaaagaagaaacataataggagagaagggaaaataatagacagggaaaaaaagagaaagataatatgtaaggacaaagaaatatatagacaatgaggcaaaataaaaaggagaggcaaaagaagagaaaggaaaaaacaagcgacagaaaacaaacaaacggcgaaagaagaaagagcagagagagaaggaaaacaaacgaagagcaaacaaccaatacccacaggagacagaagaggaaaactagcaaagacagaaggaaaaaccagcaaagcaacagcaccacataccagggtaaacgagagaagaaagaagacaaggaaagaaaagaagcgagaagaagacagaacgcagaaagacagcagggagagcacgcacgacagcagggagaagctacaggagaagaaagaaggagaacaaagaagagacaacgacaaacgaaaacaggaaagaaaagaaacgaaaaaaagaggaaag

SEQ ID NO: 84 - DNA, 1076 bp, Homo sapiens
  misc_feature: Incyte ID No 7503498CB1
ctttccaggccacgctgcgccggcttcgggaggcgccgcggcacttactggtttgcgagaaatccaacttcggcaaccacaagtcgcgccaccggcatcttgtgcagacgcactactataactacagggtttcatttctcattcctgaatgtgggatactatcggaagaactgaaaaacctggtcatgaacactggaccctattactttgtgaagaatttacctcttcatgaattaattacacctgaattcatcagtacctttataaagaaagggaaattaattttgtcactggataaagacacttatgaagaaactggacttcagggtcatccatctcagttttctggcagaaaaattatgaaatttattgtttccattgatttgatggaattatccttaaacttggattctaagaagtatgaaagaatatcttggtctttcaaagaaaagaagccattgaaatttgattttcttttggcttggcataaaacaggttcagaagaatcgacaatgatgtcatatttttccaagtaccaaattcaggagcatcagccaaaagtagcactgagcacgttgagagatctccagtgcccagtgctgcagagcagcgagctggagggaacgccagaggtgtcctgccgggctctggagctcttcgactggctcggcgccgtcttcagtaatgtcgacctaaataatgagcctaataatttcatatcaacctattgctgtcctgagccaagcacagtggtggcaaaagcttatttgtgtacaatcactggcttcatacttccagagaagatctgtctcctattggaacatctctgtcactactttgatgaaccgaagttagctccatgggttacactgtccgttcaaggctttgcagacagccctgtttcttgggaaaaaaatgaacatggttttcgaaaaggaggagaacatttatataactttgtgatttttaataatcaggactattggcttcagatggctgttggggcaaatgatcactgtccaccataaaaaataaaaattaaaaatcgtgtttacttacaaaaaaaaaaaaaaagg

SEQ ID NO: 85 - DNA, 1118 bp, Homo sapiens
  misc_feature: Incyte ID No 7504119CB1
cctgcgcgtttctttcggagcggcggtgaaggtcctgggtgaggtagggttggatggtgcttgccgcgtatcatggctgcctccggaaaactcagcacttgccgtctccctccgttgcccacgattcgagaaatcattaagttgttaagactgcaagcagcgaagcagctatcacagaatttcctcctggacttgaggctgacagataagattgtaaggaaagctggcaatctgacaaatgcttatgtttacgaagtgggccctgggccagggggaatcacaagatctattcttaatgccgacgtcgctgaacttctggtggttgaaaaggacactcgatttattcctggattacagatgctttctgatgcagcacctgggaaactgagaattgttcatggagatgtcttgacatttaaggtagaaaaggctttttcagaaagtcttaaaagaccctgggaagatgatcctccaaatgtacatattattggaaatctgccttttagtgtttcaactccactgattatcaagtggcttgaaaatatttcctgtagagatggaccttttgtttatggcagaactcagatgactttgacttttcaaaaggaagtggcagagagacttgcagccaatacaggaagcaaacagcgtagtcgcctctctgttatggctcagtacctctgcaatgttcgacacatctttacgattccaggacaagcttttgtccccaaaccagaggtggacgtgggcgtggtgcacttcactcccttgatacagcccaagatagagcagccattcaagctggtggaaaaagtggttcagaatgtatttcagttccgaaggaaatactgccatcgagggctcagagaagaactcaagcgaagaaaaagcaaaaatgaagaaaaagaagaggatgacgcagagaattacagactctagctgctgcctgggggcgagcagcctaccagatgtcgatttgcactacgtggagcttcttatataggtactcttttgtctttacagaatgacgatacaaatgccaatgaccagatgtgacttattttccttttactatacagcttggcagagaaaataaatatcatcaaataagatacaaa

SEQ ID NO: 86 - DNA, 986 bp, Homo sapiens
  misc_feature: Incyte ID No 71532805CB1
tgcgccgcctgcgtccacctccgctcgccgctctccccgcgtatccctctcatacgcagctcgcccaggtaagatgtcgtccgtggcgtcgaaggtggcggtgccggagtctgtgctccgcaagcggaagcgcgaggaacagtgggccaccgagaagaaggagaaggccctagtcgagaagaagaagtccatcgagagccgcaagctcatcttcacccgcgcaaagcagtacgccgaggaatacgatgcccaggagaaggaactggtgcagcttaagcgtgaggcccgtttgaagggtggtttctatgtcagtcctgaagcaaagctgctctttgtggtccgcatccggggtattaacgccatgcaccctaagaccaggaagatcttgcagcttctgcgattgaggcagatcttcaatggtgtgttcctcaaagtcaacaaggcgactattaacatgctacgcagggttgagccatatgttgcatatgggtacccaaacttgaagagtgtcagggagttgatctacaagaggggctatggaaagctgaacaagcagaggatccctctgtccaacaaccaagtcatcgaggagggcttgggcaagcacaacatcatctgcattgaggatcttgttcacgagatcatgactgttggcccacacttcaaggaggccaacaacttcctgtggccattcaagttgaaggcgccgctgggaggcctgaagaagaagaggaaccactatgtggagggcggtgatgccggtaaccgcgagaactacatcaatgagctcatcaaaaggatgaattaggttcacgatcaagctcattgatagtaccctgtgctctaagtgacttcttgtgctatccttattttatattagaaagttggatcgtggatgaatattcatgcagttttgatgtttcgaactagacgtgtatggaagaaatctgatcttctttccggaggtttggggattattacaaaaa

SEQ ID NO: 87 - DNA, 6546 bp, Homo sapiens
  misc_feature: Incyte ID No 5502992CB1
taaggggggaccgtggtccctcatctgataatccagatggtggaagtcatgccggcacagtccncctcgagaagatgaagaaaacagcattcagaagagacgctccaaccgccaagttaaagcgaaaaaagatatacagaggacctggatataaagatcacagatgatgaagaagaagaagaggtggatgtaactggtccaataaaacctgagcctatcctccctgaaccagtgcaagaaccagatggcgagactttgccttccatgcagttctttgtggagaatcccagtgaagaagatgcagccattgtagacaaagtgctttctatgcggattgtgaagaaggagctcccttctggaccatatactgaagcagaagaattctttgtcaagtacaagaactactcctatctgcattgtgaatgggctactatctcccaactagagaaggataagaggatacatcaaaaattaaagcgcttcaaaaccaaaatggctcagatgagacacttcttccatgaggatgaagagccctttaatccagactacgtagaggtggataggatattggatgagtctcacagtattgacaaggacaatggggagcccgttatttactacctggtgaaatggtgctctctgccctatgaggatagcacatgggagctaaaagaagatgttgatgagggcaagattcgagaatttaaacggattcagtcaaggcacccagaactcaaaagggtgaatcgtccgcaggcaagtgcctggaagaaattggagctatcacatgaatataaaaacagaaaccagctacgggaatatcagttggaaggggttaattggctgctctttaattggtataacaggcagaactgcatcctggctgatgagatgggattgggcaaaactattcagtccattgccttcttgcaggaagtatataatgtgggcatccatggtcccttcttggtcattgccccactgtccacaattactaactgggagcgagaatttaatacatggacagaaatgaacactattgtgtaccatggcagtctggccagcaggcagatgattcaacagtatgaaatgtactgcaaagattcacggggacgcctcatcccaggcgcatacaagtttgacgctctgatcaccacttttgagatgattttgtcagattgtcctgagcttcgtgaaattgaatggcgttgtgttatcattgatgaagcccatcgactgaaaaaccgtaattgcaagctgcttgatagtctcaagcacatggacctggaacacaaggtgctactcacaggaacaccattgcaaaatactgtagaagaactgtttagcttgcttcatttcttggaaccgtcacaatttccctcagaatcagagtttctcaaggactttggggatctcaagacagaggaacaggttcaaaagctacaggccattcttaagccaatgatgctgagaagactcaaagaggatgttgaaaaaaacttggcacccaaacaggaaacaattattgaagtagagctgactaatatccagaagaaatactatcgggctattttggagaagaatttctccttcctttccaaaggggcaggtcataccaacatgcctaatctacttaacacaatgatggagttgcgcaagtgctgcaaccacccatatctcatcaatggtgctgaagaaaaaatcctaacagaattccgtgaagcttgccatattatacctcatgactttcacctgcaggccatggttcgttcagccggcaaactggttcttattgacaagttgcttccaaagcttaaagctggtggccataaagttctgatcttctctcagatggtacgctgcctagacatcctagaggattatttaatccagaggaggtacttatatgaacgtattgatgggcgagttagaggcaaccttcgacaggctgccattgaccgcttcagcaagcctgactcagaccgctttgtcttcttactgtgtacccgggctggtggacttggtattaatcttacagctgctgatacctgcatcatctttgattcagactggaatccacaaaatgacctgcaggcccaggcacgatgtcatcgaattgggcagagcaaagctgtgaaggtgtaccgcctcatcactcgtaattcctacgagagagagatgtttgataaggccagcctcaagttggggttggataaggctgtgcttcaatccatgagtggtcgggatggcaacattactggaatccaacagttctctaagaaggagattgaagatcttttaagaaaaggagcatatgcagccatcatggaggaagatgatgaaggctccaagttttgtgaagaggacattgaccagatcttgttaagacgaactacaaccatcaccattgaatctgaaggaaaaggttccacctttgctaaggcaagctttgttgcttctgaaaacaggacagatatttctttggatgaccccaacttttggcaaaagtgggccaaaaaggctgacctagacatggatctgctcaacagcaagaataatttggtaattgacacacctagagtacgaaaacaaacgcgccactttagcactctgaaagatgatgacctggtggaattctctgatttggaaagtgaggatgatgagcggccacgctcccgcagacatgaccgtcatcatgcctatgggcgcactgactgctttcgggtggaaaagcatctcctggtatatggttggggacgatggcgagatattttatctcatggacgcttcaagcgacgtatgactgaacgagatgtggagaccatttgtcgggccattctcgtgtactgtcttctacactaccgtggggatgaaaatattaaaggcttcatctgggacttgattagcccagctgaaaatggcaagacaaaagaattgcagaatcattcaggtctatctatccctgtgcctcgtggacgcaaaggaaaaaaagtaaagtcacaaagcacttttgatatccataaggcagattggatccggaaatataaccctgacactttgttccaagatgaaagttataagaagcacttgaaacatcagtgtaacaaggtactgttgcgggtacgaatgctatactacctgaggcaggaggttattggagaccaagcagaaaaggtgttagggggtgcgattgccagtgagattgacatatggttcccagtagtggatcaactggaggttccaacaacttggtgggacagtgaggctgacaagtcgctgctcattggagtctttaaacatggctatgagaaatataataccatgagggcagacccagccttatgtttcctagaaaaggctggccgaccagatgacaaagcaattgcagcagaacatcgagtgttggataacttctctgacatagtagaaggggttgactttgataaagattgtgaagatcctgaatataaaccactccaaggtcccccaaaggaccaagatgatgagggtgatcccttgatgatgatggatgaggagatctcagtgattgatggagatgaagcccaggtgacccaacagccaggccatttattctggcctccgggctctgccctaacagctaggcttcggcgtctagtaacagcgtatcagcgcagctacaagagagaacaaatgaagatagaggctgcagaacgtggggaccggcgaaggcggcgttgtgaagcagccttcaagctgaaagaaattgcacggcgggagaaacaacaacgatggacaaggcgtgaacaaactgatttttatcgagtggtgtctacgtttggtgtggaatatgaccctgacaccatgcagttccattgggatcgcttccgcacttttgctcgactagacaaaaagacagatgaaagccttaccaagtacttccatggctttgtggccatgtgccgccaagtatgccgccttcccccagcagctggagatgaaccccccgaccctaacctgttcattgagcccatcactgaggagagagcctcacggactctctaccgtatagaattgcttcggcgcttacgggaacaagttttatgccacccccttttggaagatcggctggcattgtgtcagcctcctggtcctgaattgcccaaatggtgggagcctgttcggcatgatggggagcttctaagaggggcagcccgccatggggtgagccaaacagactgcaacatcatgcaggacccagacttctcttttctggctgcccgtatgaattatatgcagaaccatcaagcaggagcaccagctccatccttgtcacgctgctctactccactgctgcaccagcagtatacctcacgcactgcctcaccactgcccctgcgcccagatgctcctgttgaaaagtcacccgaggagacagctacccaggtccccagtctggagagtctgactttaaagctagagcacgaggtggtggccaggagccgaccaaccccacaagactatgagatgcgagtatccccctctgatactacccctctggtttcccggagtgttccaccagtcaaactggaggatgaggatgattcggactctgagctggacttgagcaagctgtcaccatcttcttcttcttcctcatcctcatccagctccagctccagcactgatgagagtgaggatgagaaggaagagaagctaactgaccagtcccgctcaaagctctatgatgaagagagtctcctgtccctcactatgtcccaagatggattcccaaatgaagatggagaacaaatgacccctgagcttctgctactgcaggaaagacaaagagcctctgagtggcccaaggatcgtgtcctgataaaccgtattgacctcgtctgccaggctgtactctcagggaagtggccttctagccgtaggagccaggaaatggtaacaggaggaattttggggccaggcaaccacttgctagacagtccctcattgactcctggagaatatggtgactctccagtccccacaccacgaagtagtagtgcagcttccatggcagaggaggaagcatctgcagtcagcacagcggcagcccagttcaccaaacttcgccgaggcatggatgaaaaggagtttacagttcaaatcaaagatgaggaaggattgaagttaacattccagaagcacaagttgatggcgaatggagtaatgggagatggacatccactgtttcataagaagaaggggaacagaaagaagctagtagagctggaggtggagtgcatggaagagcctaatcaccttgatgtggacctggagacccggatccctgtcatcaataaggtggatggtactttgctggtgggtgaggatgcccctcgccgggctgaactggagatgtggttacagggtcatccagagtttgctgttgatccccgatttctagcgtatatggaggatcgcagaaaacagaagtggcaaagatgtaaaaaaaataataaggcagaattgaactgtttgggaatggaaccagtacagacagctaactctagaaatgggaaaaagggtcatcacactgaaacggtgttcaaccgggttttgccagggcctattgcaccagagagcagcaagaagcgggcccgtaggatgcgaccagacctttctaagatgatggccctcatgcagggtggaagcactgggtctctatctctgcataacacgttccaacacagcagtagtggcctacagtctgtgtcatctttgggtcacagcagtgccacttctgcatctttgccttttatgccatttgtgatgggtggtgcaccatcatcccctcatgtagactccagcaccatgcttcatcaccaccaccaccacccccacccccaccatcaccaccatcaccatccaggcttgagagcccctggctacccctcttcaccagtgactaccgcctctggtactaccttgcggttgccaccactgcaacctgaggaggatgacgatgaggatgaagaagatgatgatgacttatctcagggctatgatagctcagaaagggacttctcactcattgatgatcctatgatgccagctaactcagactccagtgaagatgctgatgactgaagccccagcatgggccccattgcttgggcggctgctgtattttcatttactctggcccttggactatggaaacgtgggaggggcaggggagatgtggggaagtccaggactccaggaggtgaaaaggaaaaaaaaaaaaaaatgtacctgattgctcccaattatgagaggattgggtgggcaggggaactcctaaaataatacatgaccacttcctcatttctggggaaggaaaggagactagagcagctggtgtgctcacccctccctagtcacctccattaaccacagactatgtagcgctggccctagcctctggcagagcctgttcctggccgaactgtggatacagctggagggtcaggaactgttaccttctttccccttggcattaataaatttaagttaatccttgaaaaa

SEQ ID NO: 88 - DNA, 3737 bp, Homo sapiens
  misc_feature: Incyte ID No 7503828CB1
ggaggcggaggaggcgggagctgagctgagtggggcgggcggcggcggggcccgagccggagaagatggcggtgcggaagaaggacggcggccccaacgtgaagtactacgaggccgcggacaccgtgacccagttcgacaacgtgcggctgtggctcggcaagaactacaagaagtatatacaagctgaaccacccaccaacaagtccctgtctagcctggttgtacagttgctacaatttcaggaagaagtttttggcaaacatgtcagcaatgcaccgctcactaaactgccgatcaaatgtttcctagatttcaaagcgggaggctccttgtgccacattcttgcagctgcctacaaattcaagagtgaccagggatggcggcgttacgatttccagaatccatcacgcatggaccgcaatgtggaaatgtttatgaccattgagaagtccttggtgcagaataattgcctgtctcgacctaacatttttctgtgcccagaaattgagcccaaactactagggaaattaaaggacattatcaagagacaccagggaacagtcactgaggataagaacaatgcctcccatgttgtgtatcctgtcccggggaatctagaagaagaggaatgggtacgaccagtcatgaagagggataagcaggttcttctgcactggggctactatcctgacagttacgacacgtggatcccagcgagtgaaattgaggcatctgtggaagatgctccaactcctgagaaacctaggaaggttcatgcaaagtggatcctggacaccgacaccttcaatgaatggatgaatgaggaagactatgaagtaaatgatgacaaaaaccctgtctcccgccgaaagaagatttcagccaagacactgacagatgaggtgaacagcccagattcagatcgacgggacaagaaggggggaaactataagaagaggaagcgctccccctctccttcaccaaccccagaagcaaagaagaaaaatgctaagaaaggtccctcaacaccttacactaagtcaaagcgtggccacagagaagaggagcaagaagacctgacaaaggacatggacgagccctcaccagtccccaatgtagaagaggtgacacttcccaaaacagtcaacacaaagaaagactcagagtcggccccagtcaaaggcggcaccatgaccgacctggatgaacaggaagatgaaagcatggagacgacgggcaaggatgaggatgagaacagtacggggaacaagggagagcagaccaagaatccagacctgcatgaggacaatgtgactgaacagacccaccacatcatcattcccagctacgctgcctggtttgactacaatagtgttcatgccattgagcggagggctctccccgagttcttcaacggcaagaacaagtccaagactccagagatctacctggcctatcgaaactttatgattgacacttaccgactgaacccccaagagtatcttacctctaccgcctgccgccgaaacctagcgggtgatgtctgtgccatcatgagggtccatgccttcctagaacagtggggtcttattaactaccaggtggatgctgagagtcgaccaaccccaatggggcctccgcctacctctcacttccatgtcttggctgacacaccatcagggctggtgcctctgcagcccaagacacctcagcagacctctgcttcccaacaaatgctcaactttcctgacaaaggcaaagagaaaccaacagacatgcaaaactttgggctgcgcacagacatgtacacaaaaaagaatgttccctccaagagcaaggctgcagccagtgccactcgtgagtggacagaacaggaaaccctgcttctcctggaggcactggaaatgtacaaagatgactggaacaaagtgtccgagcatgtgggaagccgcacacaggacgagtgcatcttgcattttcttcgtcttcccattgaagacccatacctggaggactcagaggcctccctaggccccctggcctaccaacccatccccttcagtcagtcgggcaaccctgttatgagcactgttgccttcctggcctctgtcgtcgatccccgagtcgcctctgctgctgcaaagtcagccctagaggagttctccaaaatgaaggaagaggtacccacggccttggtggaggcccatgttcgaaaagtggaagaagcagccaaagtaacaggcaaggcggaccctgccttcggtctggaaagcagtggcattgcaggaaccacctctgatgagcctgagcggattgaggagagcgggaatgacgaggctcgggtggaaggccaggccacagatgagaagaaggagcccaaggaaccccgagaaggagggggtgctatagaggaggaagcaaaagagaaaaccagcgaggctcccaagaaggatgaggagaaagggaaagaaggcgacagtgagaaggagtccgagaagagtgatggagacccaatagtcgatcctgagaaggagaaggagccaaaggaagggcaggaggaagtgctgaaggaagtggtggagtctgagggggaaaggaagacaaaggtggagcgggacattggcgagggcaacctctccaccgctgctgccgccgccctggccgccgccgcagtgaaagctaagcacttggctgctgttgaggaaaggaagatcaaatctttggtggccctgctggtggagacccagatgaaaaagttggagatcaaacttcggcactttgaggagctggagactatcatggaccgggagcgagaagcactggagtatcagaggcagcagctcctggccgacagacaagccttccacatggagcagctgaagtatgcggagatgagggctcggcagcagcacttccaacagatgcaccaacagcagcagcagccaccaccagccctgcccccaggctcccagcctatccccccaacaggggctgctgggccacccgcagtccatggcttggctgtggctccagcctctgtagtccctgctcctgctggcagtggggcccctccaggaagtttgggcccttctgaacagattgggcaggcagggtcaactgcagggccacagcagcagcaaccagctggagccccccagcctggggcagtcccaccaggggttcccccccctggaccccatggcccctcaccgttccccaaccaacaaactcctccctcaatgatgccaggggcagtgccaggcagcgggcacccaggcgtggcggacccaggcacccccctgcctccagaccccacagccccgagcccaggcacggtcacccctgtgccacctccacagtgaggagccagccagacatctctccccctcaccccctgtggacatcacggttccaggaacagcccttcccccaccactgggaccctccccagcctggagagttcatcactacgtaaggaaagctccttccgcccctccaaagccctcaccatgcctaacagaggcatgcatttttatatcagattattcaaggacttctgtttaaaagatgtttataatgtctgggagagaggataggatgggaatgctgccctaaaggaagggctggtgaaaggtgtttatacaaggttctattaaccacttctaagggtacacctccctccaaactactgcattttctatggattaaaaaaaaaaaaaaaaaattctgcggcg

SEQ ID NO: 89 - DNA, 2171 bp, Homo sapiens
  misc_feature: Incyte ID No 2647325CB1
ccgggagtgggagcgccgggacttaattacgtatttatccagtgttgcgtggctccgcgggggggcgctgcggccgtggggggtgggctgcattcattccctctgggagctggggcttgcagtcgggcggggtgggttgcggcagcagggtggaggcctacgtattttcgtgggggtggggttgcgacgcagagtttaccatatttctccagggttgctccggccgcagaagttggcaaggttgtgtgtaaggtcgggtggggtggggtcagctctcccgattaatgcagctatttgataagagcaacgtcgcgggacgcttgccgtctttatgggggcggggagaaggggcgcgggtgtcgctgcgggagagttaccgtatttgcgttgagcagcgcagactgtagagcaaagagctgcggccggagaccggaggagcaggggctcagctgggctggactggtgattgcagctggaagtgcccatgaccgagctggcgtcctccgggggcgggtcccctgcgggggacggggaggagggtctgggggacgagcgaggcctggtcatccaccaccctgcagaggagcagccctaccgctgcccgctgtgcggccagaccttctcgcagcagcccagcctggtgcggcaccagaaggcgcacgccggagcgggccgcgcggctgccttcgtgtgtcccgagtgcggcaaggccttcagcgtcaagcacaacctcgaggtgcaccagcgcacgcacaccggggagcggcccttcccctgccccgagtgcggccgctgcttcagcctcaagcagaacctgctcacgcaccagcgcatccacagcggcgagaagccgcaccagtgcgcgcagtgcggccgctgcttccgcgagccgcgcttcctgctcaaccaccagcgcacccacgcgcgcatgcccgcgccgcacccgcgccgccccggcgtcttcggggagcggcggccctacttctgcccccgctgcggcaagagcttcgcgcgcgagggctcgctcaagacccatcagcgcagccacggccacgggcccgagggccaggcggcccacttaggacgcgtgctatgatgcgcccggggcctgctgccgacggctgcttcccgccccgcacgtgcgtccccgacccctggagatggccctgggccgcagctccctctctagatgggatcccgggagaggggcagcgctggcttgggctcttgaaggtgtgggccgccgccaggctccggccgcccgctcgcgttcctccctcgggaccctctggctggctgcgcacagctgcttccggctcctgccatggttctccttctctggcccatccggcctagagcagttgcagagtgggcaggatcttaagacccgataggtgcagaacccatctggacacggagaccaggaatggagttccatggaggcctggctggcactgcacccgggcatgaggacacatccagtaagaagacctgcctcaagaggtgcactgcggtgaccagtggaggtgactggttggagcctggaattggaagcagattccaagctctggtggacaaactctccaggcctggtgggaatcacagctggggcagacctcatcctggctgcctggccacaggcccccactctctgccactggtggtaggacgatgcctgtgtggagagctggcttctctgctcccgcctggtccaccacttggctagagttcagagacaggaagtgattggtctaagctaacacagcaagttggtggcagacctggttctagaggcaaaaccttcttccagatgtgaatgaaacctgcaggcttcattttcctttctgagcagtgcttcttagctctttggagacacgaagcccttggaaaatctgatgaaggttacggaccttccctaggaaaacagataactgacgtagactcaaaaaccccaagcaatttcaggagccactggactccctgaatgaaacccatccctggactccaggctaagaacctcagccctggggacttcacctgctgccctttccttacctgtcacacattgagccccgagtcaaggccactgtacaagtagtgcccctccctccccctggccaagcctccttcccttgttcaggaataaagaattccgaggagccctttttagt

SEQ ID NO: 90 - DNA, 1565 bp, Homo sapiens
  misc_feature: Incyte ID No 7495416CB1
agcagaggcactgggcgcagcggagacctccaagccggccccaggagacgggccgcgaggttaggttggaactcggcggcaaggaccttgaacgccgggctggggcggctccaggtgcttgggagtcgggggactgcggaggacgccaagtctgagctcgccgccctctccgagccgtttgggccggacgcctgggtccttcgggctccgccccagggggtggggctatatgttcggaccaatgaccggccgtccctgcggtgccgccccctccccggccccggagccgcggcctcgctgagtgcccagccgcccggcgcccaggcctggggcaccgcgagtgccgaaccttcggctggacaccaagatgcctggcgaacagcaggcagaggaagaggaggaggaagagatgcaggaggagatggtgctgctggtgaagggtgaggaggatgagggtgaggagaagtatgaggtggtgaaactcaagatccccatggacaacaaggaggtcccgggcgaggcgcccgcgccgtccgccgacccggcgcgtccccacgcgtgccccgactgcggccgcgccttcgcgcgccgctccacgctggcgaagcacgcgcgcacgcacacgggcgaacggcccttcgggtgcaccgagtgcgggcggcgcttctcacagaagtcggcgctgaccaaacacggccgcacgcacacgggcgagcggccctacgagtgccccgagtgcgacaaacgcttctcggccgcctcgaacctgcggcagcaccggcggcggcacacgggcgagaagccgtacgcatgcgcgcactgcggccgccgcttcgcgcagagctccaactacgcacagcacctgcgcgtgcacacgggcgagaagccgtacgcgtgcccggactgcggacgcgcctttggcggcagctcgtgcctggcgcgccaccgacgcacgcacacgggcgagcggccctacgcttgcgccgactgcggcacgcgcttcgctcagagctcggcgctggccaagcaccggcgcgtgcacacgggcgagaagccgcaccgctgcgctgtgtgtggccgtcgcttcggccaccgctccaacctggcggagcacgcgcgcacgcacacaggcgagcggccctacccctgcgccgagtgcggccgccgcttccgcctaagctcgcacttcattcgccaccgacgcgcgcacatgcggcgccgcctgtatatttgcgccggctgcggcagggacttcaagctgccccctggcgccacggccgccactgccaccgagcgttgcccggagtgtgagggcagctgagtcccgcagggctgcggaggggcgcgctggggcttcgacctggctgcactaacccaggctcctcctcgccccggcctccgggtctgggaaattgaggggacggcaggcccggctgccctggaactgggagacagggagaatcccctgccggggtccctggaaacagtgcccaccccacatcactacattccctcggcccgtgttagtgaataaagtattatatcctcaccccaaaaaaaaaaaa

SEQ ID NO: 91 - DNA, 5121 bp, Homo sapiens
  misc_feature: Incyte ID No 8096177CB1
gggggaggaggcggggctgcgggggagagcgactgcgccaggtccgcgacaaatgaattagacacaattatccacgggagactcggttttcctctctgtaaattacggatattaggaatacttgccgcctggggttgttttgagcactttcattgtttaggtgctgtatttattgggtgtgcgttccctgggagagcccgagggagacggctcctgcaccggccccaagggcctctctgcccgtggagtgcaggagcgaaggggctggcctagctgaccaccagggcccctgctggctctgaccgccctcctatcccaagtaattatttacacgtttcgcgttcgcttatgtattatgtgtaatcatggcacagtatcctgaagccgtcggagtctgtgtgctggtgcaaggtctgggacaaatttaagaagagagagaacctaaattggtaataaatcaataagaaatatttacattcactcagtgaaaagtgtcatgtctgagctcagcatatatcggagccacacatggacacctctccttcctccactaagagcggaaaatgaacaaatcccaggaacaagtgtcattcaaggatgtatgtgtggacttcactcaggaagagtggtatctgctggaccctgctcagaagattctatacagagatgtgatcctggaaaattatagcaatcttgtctcagtagggtattgcattactaaaccagaagtgatctttaagatcgagcaaggagaagagccctggatattagaaaaaggattcccaagccagtgccacccagaaaggaaatggaaagttgatgacgtgttagagagcagccaggaaaatgaagatgaccatttttgggagcttctattccacaacaacaaaacagtaagtgtagaaaatggagatagaggaagcaaaactttcaatttgggcacagaccctgtttctttaagaaattatccctataaaatatgtgactcatgtgaaatgaatttgaaaaatatttcgggcttaattattagtaaaaagaactgttccagaaagaagcctgatgagtttaatgtatgtgagaaattgctccttgatattaggcatgagaaaatccctattggagagaagtcttataaatatgatcaaaaaaggaatgccattaattatcaccaggatctcagtcagccaagttttggccaatcttttgagtatagtaaaaatggacaaggcttccatgatgaggcagcattttttacaaataagagatctcagataggagagacagtctgtaaatataacgaatgtggaagaaccttcattgaaagtttaaagctgaatatatctcaaagacctcatttggaaatggagccgtatggatgcagtatttgcgggaagtccttctgcatgaatttaaggtttggacatcagagagctcttacaaaggacaatccttatgaatataatgaatatggggaaatcttctgtgacaattcagctttcattatccatcagggagcttacacaagaaagattctccgtgaatataaagtgagtgacaaaacctgggaaaagtcagctctcttaaaacatcaaatagtacacatggggggaaagtcttatgattacaatgaaaatgggagtaatttcagcaagaagtcacatcttacccagcttcggagagctcacacaggagaaaaaacctttgaatgtggtgaatgtgggaaaaccttctgggagaagtcaaacctcactcaacatcagagaacacacacaggagagaagccctatgaatgtactgaatgtgggaaagccttttgccagaaaccacacctgaccaaccatcagcgaacacatacaggagaaaaaccctatgaatgtaagcaatgtggaaaaacattctgtgtgaagtcaaacctcactgaacatcagagaacacacacaggggagaagccctatgaatgtaatgcatgtgggaaatccttctgccacagatcagccctcactgtgcatcagagaacacacacaggggagaaaccgtttatatgtaatgaatgtggaaaatccttctgtgtgaagtcaaacctcattgtacatcaaagaactcacactggggagaaaccatataagtgtaatgaatgtgggaaaaccttctgtgaaaaatcagctctcactaaacatcagaggactcacacaggggagaagccgtatgagtgtaatgcatgtgggaagacctttagtcagaggtcagtgctcaccaaacatcagagaattcacacaagggtgaaagctctttcaacatcctgaatgttagaagccttcatacacttgtgaaattggttatacagtttcaaaaaaggagatcagagaaagccaaagaatgtcagaaatttgtagaaaatgacttcttgtttgaatatgtaaaagctttcaagaaaaattaaaacttttcattagaaaatttgtactgaggggaattctattcatctaagtaatatggtgaaaatatttatctggaatttatgttgtttagtgttatattccagacgtgataccaaaattttgttgcaaatataatggacaatatttatttatacccatattcacagtggaatctgaagcttataaaagttgaatgacagcagcattaaacatatatgtgaagagtccccgatgtttgtagaccttatgtgagtatgcaaatatataaatttgagtatgcttgatttgtatattggaactcaacatgatcataaggagaagatacgtacccttaattgagtaactactatggtatttgttaatattttctacactaaataccattggtgtctttataggttgacataattatatatgtgtatgtacatatgtttgtgtgtatatgtaaatatatttctacacacatacttaaatatagtgatgtgctagtataacctcatactgacttaaaagttctgattgttaaattttaaggaattttgtgagccagttattaaaagcagtcattatttaaaatatgtaaacttacagttaaaaacaaaggtaataaatactcagcactcatcacttcctaattattttgctacatttcactattacctttgctgttttacttatttaatctgtatgatgaaaatactgtataatagtgtgcactgcacatctgtctcttcccagctccacattcagtgctgtcttggtagcttggacttagtgggagtgtttacaccatggaaattgacaaactataaatcagggttttaattttctcagagaacctgctgtcaaatatttacagcacatcactgtgtatatatgaaaacgtatttggcaatacaaaccacataccccttctatttcctgacataaataaatggctatggccatttacagctgaaccacgtcttcaagaaagaagccaaaaatatttccgtgaggtttttaactacctctgaatctgtcctactctaaatactaccggagtctctttgtaggttggccagtatatgtttttagtgaaatattatttcacaaagaactatatcacgtacctttcctctgactgtttcctggcatatatgcatgaatatggccattattgaactatcacttcagtaaagaagttaaacagtacttttctgaggtttttcagctacctctgggtcattctgtaatgtaaatgttgttaataagaatggtttttacataaattatgcaaaggttaacaagcagtaacactgcactcctcaaaaagtggcggtatgtaatgaaaggcccttttgatatccttgatttttcattgtgtatctgtttgggcacggtctatgtaacactagttctgcgtattagtattttagagtatctctgcctcccttgtcctgttgtttcttttgcccccttggaacacattggtcagcagttctaagagacactgcccacatgatggccattccctacttcatccttgctgagctaaattttatatttttgtgcatccttctcccagatgacttaggtggtaagtccagattagtcaaagctaatcatggaagttccattttaatgattctgttggggtgaacttgggagcaatgagatgtttgggaagtattgtgtagtacttctgggaaagatctccttgatacaacattgtcatgacatgagagactctgctgggctttttcatgtctgtaacatggtattggcttatcgtttttatctctgaagggcagtagcctgaagataacagtgcacaaggtgggaaaagccagctcagaggtgacgttgccgagctactctgctctctatacctgttctctactgggactttttataaccctcaataactgttttttatttggtcttagggctgtctgatacttagagctgaaggcattccagctgacacagaggaatatttttctaagtgttaatgttctatatggtaattagggggaagaattatttcttttcacaagttaatatagggatggctgtttgtatcagccatggttctttctggtggaaaacagaattctccaactaaaaatattttaatggcagactgattacagtggtgtgggccagaaacaagggacagtgaaacacccagagacttgtatcagcaggaagccattgccattctgagccttgaagggcaaggagggaaacagtgttaccagagcccagtaagaactgctgtcatgaaggaggggccaccttgtaagagacatcattactaccagaactgtggtgccaaattgctggtgtctctctttggagaaaccaaccagatacatctgctggagagcccaggtgggcacagagaagggtggagagagaatctgggaagagaaatggagaataagcagcacagtgttattcatttctgtaaattcctatgtagaaggctcagtgttagaaataaagttattctactagttgcaagttaagtgtttctgtttgttctgctttcctgttagcataagtaaactccctttggaactacacaggtatgtctctccttcaacatgtgtgaagcagacattatattaaattacattattcatacctccctgtggtgtttcttattgtatgtggtacagcgaagcagctctgattc

SEQ ID NO: 92 - DNA, 2626 bp, Homo sapiens
  misc_feature: Incyte ID No 666763CB1
ctctttttatttacacgggagcactccacagtgttttgcagctttcctatttcactaaactacagcccattctgctcttataagtagcagcgttgccctcgattggctgcacccgagttctgaccctcctcctccagcgagggcctcggcagccagcaagatgggctcccgctcccggaatggactcggaaactttaactgcggctccacctgctggtgtggattaggacaaaggcggagaacaaacctcgtgtgcaagctgaacacaaggaccaccatcctggcagcaacgaaccccaaaggccagtacgacccccaggagtccgtgtctgtgaacattgccctcggcagcccactcttaagtcgatttgacctgatcctggttttgcttgataccaagaatgaagactgggatcgtatcatttcctcctttatcttagaaaataaaggttacccaagcaaatcagagaagctctggagcatggaaaagatgaaaacctatttctgcctcataaggaatctgcagcccacactgtctgatgtgggcaatcaggttcttctccggtactaccagatgcaaaggcagagtgattcccggaacgctgcccggaccaccattcggctgttggaaagcttgatacgattagcagaagctcatgctcgcctgatgtttcgtgatactgtaactctggaagacgctattacggtggtgtcagtcatggagtcctcaatgcagggaggtgcactgctaggaggtgtgaatgccctccacacttcctttcctgaaaaccctggagagcagtaccagagacagtgtgaacttattctggaaaagctagagctgcagagcctcttgagtgaagagcttagaagacttgaaaggttacagaatcagagtgtgcaccaatcccaaccacgggtattggaggtagagactactccaggatccttgagaaatggtccaggggaagaatcaaacttcagaacttcatcacagcaggaaatcaactatagcacacatatcttctctcctggaggcagccccgagggaagcccagttctagatcccccaccgcatctggagcctaatagatcaacaagtaggaaacattcagctcagcacaaaaataacagagatgacagtttagattggtttgatttcatggcaactcatcagagtgaacctaaaaacactgttgttgtgtctcctcatcccaaaacatctggagaaaatatggcttcgaagatctctaacagcacatctcagggtaaggagaagagtgagccaggccaaaggagcaaagtggacattgggttgcttccatcaccaggagagacaggtgttccatggagggcagacaatgtggaaagtaacaagaaaaaaaggctagcactagattctgaagcagcagtctctgctgataaaccagactcagtactgactcatcatgtccccaggaacctgcagaagctgtgcaaagagagggcccagaagttgtgcagaaatagcaccagggtgcctgcacagtgcacagtcccttcccatcctcagtccactcctgtacatagcccagacagaatgctggactcacccaaaagaaagagaccgaaatcccttgcgcaagtggaagagcctgcaattgaaaatgttaagcctccaggttcccctgtggccaaactggcaaaatttactttcaagcagaagtcaaaactgatccactcctttgaagatcacagccatgtgtcacctggtgcaactaaaatagcagttcatagtcctaaaatttcccagcgtagaacaagaagagacgcagccttgccggtgaagcgtccaggaaagttaacatctaccccaggaaaccagatctccagtcagccacagggtgagacaaaggaggtgtcgcagcagccaccagagaaacacggaccaagagagaaggtgatgtgtgcccctgagaagaggattattcagcctgaattagagcttgggaacgagactgggtgtgctcatcttacttgtgagggagacaaaaaggaagaggtttcaggcagtaataaaagcggcaaggttcatgcctgcacattagccagattggcaaacttctgctttactcccccatcggaatccaaatcaaaatcccctcctcctgaaaggaagaaccgaggtgagagaggcccaagctcccctcctacaaccacagctccaatgcgtgtcagtaaaaggaaatcttttcagctccgtgggtccaccgagaaactgattgtttccaaagaatccctcttcactttaccagaactaggtgatgaagcatttgattgtgactgggatgaagagatgagaaaaaagtcatagttgggaaaagctttctggtcaaatctcaccttcttcaactccacagaggaccttcaggatatcaatatggtatttataaatgtatagaacaattggccatattgaggatcactctgaatactggctcccccttaaggctttctaatttcaggttaatcttcatgacttaaaaagttgtataatcagttgaggtcagtgtgataccagcagctgagctgaattaattatgttgtgcttaattttacaaatggagtacttgtattcctgttcctg

SEQ ID NO: 93 - DNA, 1620 bp, Homo sapiens
  misc_feature: Incyte ID No 7504091CB1
gcgaccgttccggcggccattgcgaaaacttccccacggctactgcgtccacgtggcggtggcgtggggactccctgaaagcagagcggcagggcgcccggaagtcgtgagtcgagtcttcccgggctaatccatgccgggttggaggctgctgacgcaggtcggcgcccaggtgctgggtcgactcggggacggcctgggtgctgccctgggcccggggaacagaacacacatctggctttttgttagaggtcttcatggaaagagtggtacatggtgggatgagcatctttctgaagaaaatgtcccattcattaagcagttggtctctgatgaagataaagcccaattagcaagtaaactgtgtcctctgaaagatgaaccatggcctatacatccttgggaaccaggttcctttagagttggtcttattgccttgaagctgggcatgatgcctttatggaccaaggatggtcaaaagcatgtggtcacattacttcagaaagctacatccatattggaattttaccgggaacttggattgccgccgaaacagacagttaaaatctttaatataacagataatgctgcaattaaaccaggcactcctctttatgctgctcactttcgtccaggacagtatgtggatgtcacagccaaaactattggtaaaggttttcaaggtgtcatgaaaagatggggatttaaaggccagcctgctacgcatggtcaaacgaaaacccacaggagacctggagctgttgcaactggtgatattggcagagtctggcctggaactaaaatgcctggaaaaatgggaaacatatacaggacagaatatggactgaaagtgtggagaataaacacaaagcacaacataatctatgtaaatggctctgtacctggacataaaaattgcttagtaaaggtcaaagattctaaactgcctgcatataaggatctcggtaaaaatctaccattccctacatattttcctgatggagatgaagaggaactgccagaagatttgtatgatgaaaacgtgtgtcagcccggtgcgccttctattacatttgcctaacatctttggacgtggcagaaccttacatattctgtgagcttcgatgagccagagtgatatcataaccaccagaaatcatactctcctttcttagtcacaacaaaatcacacatgtcatctttgtcaagggcataaatatatcattcatacccccattaaattttgttagaaaaattaccacattaaatatatgagttaagtagattggatttgctgaaattggtgttgggcatattagcaaaatattcttaatttgtggactcgattcttttttactacatatttcccaagttatcttaagatgtctgtaaatttaacttttattaaagttttgtcaatctttgtgaaatagtggttgtggaacagtagaaaaccatatggggactatagtgcaacctatttgggtaaagaaaccatttgctaaaatggagaaagtaaatagatttttatttaaattacagaaacatgttaaaggccggacaaaggaaagacaataaaatcataaattatcaaaaaaaaaaaaaaaaaattctgcggtc

SEQ ID NO: 94 - DNA, 1444 bp, Homo sapiens
  misc_feature: Incyte ID No 7503568CB1
gttccgcccccggcctcccgcccttccccttcccgcccgctccccttttcccctcagtcgcctcgcgcctgcagtttttggctttcacccccaaccagtgaccaaagacttgaccactcaaagtccagctccccagaacactgctcgacatggacaccggtgtgattgaaggtggattaaatgtcactctcaccatccggctacttatgcatggaaaggaagttggcagtatcatcggaaagaaaggagaatcagttaagaagatgcgcgaggagagtggtgcacgtatcaacatctcagaagggaattgtcctgagagaattatcactttggctggacccactaatgccatcttcaaagcctttgctatgatcattgacaaactggaagaggacataagcagctctatgaccaatagcacagctgccagtagacccccggtcaccctgaggctggtggtccctgctagtcagtgtggctctctcattggaaaaggtggatgcaagatcaaggaaatacgagagagtacaggggctcaggtccaggtggcaggggatatgctacccaactcaactgagcgggccatcactattgctggcattccacaatccatcattgagtgtgtcaaacagatctgcgtggtcatgttggagtcccccccgaagggcgtgaccatcccgtaccggcccaagccgtccagctctccggtcatctttgcaggtggtcagttgaccaagctgcaccagttggcaatgcaacagtctcattttcccatgacgcatggcaacaccggattcagtggcattgaatccagctctccagaggtgaaaggctattgggcaggtttggatgcatctgctcagactacttctcatgaactcaccattccaaacgatttgattggctgcataatcgggcgtcaaggcgccaaaatcaatgagatccgtcagatgtctggggcgcagatcaaaattgcgaacccagtggaaggatctactgataggcaggttaccatcactggatctgctgccagcattagcctggctcaatatctaatcaatgtcaggctttcctcggagacgggtggcatggggagcagctagaacaatgcagattcatccataatccctttctgctgttcaccaccacccatgatccatctgtgtagtttctgaacagtcagcgattccaggttttaaatagtttgtaaattttcagtttctacacactttatcatccactcgtgattttttaattaaagcgttttaattcctttctctgttcagctgttgatgctgagatccatatttagttttataagcttctccctggtttttttttttttttggttttttttttttggctcatgaatttttctgtttgtcatggaaatgtaagagtggaatattaatacatttcagtttagttctgtaatgtcaggaat

SEQ ID NO: 95 - DNA, 2562 bp, Homo sapiens
  misc_feature: Incyte ID No 7504101CB1
ctgaggggagcccgcgcctccgccgcctgagaggaggtcgagctgccgccggggcgatgctggaggagctggagtgcggggcgcccggcgccaggggagccgccacagccatggattgcaaagatagaccagcttttccagttaagaagttaatacaagcccgtctgccgtttaagcgcctgaatcttgtcccaaaggggaaagccgatgacatgtcagacgatcagggtacttctgtgcaaagtaaaagccccgatttagaggcctctttggacaccttggaaaacaactgtcatgtgggttctgacatagactttagaccgaaacttgtcaacgggaagggtcccttagataactttttaagaaatagaatcgaaaccagtattggccagagcacagtcatcattgatttgacagaggactcgaatgagcagccagacagtcttgtggaccacaataaactaaattctgaagcctctccctccagggaggcaataaatggccagcgagaagacactggggatcagcaggggttgttgaaggccattcagaacgacaagttggcatttcctggagagaccctttcagacattccttgcaaaacagaggaggagggtgttggctgtggaggtgcagggaggagaggcgactcccaggaatgttcgccacggagctgcccggagctgacgagtggcccgagaatgtgccccagaaaggagcaggacagttggagtgaagctgggggcatcctgttcaaagggaaggtgcctatggtggtcttgcaggacatcttggctgtgagaccaccgcaaatcaagtcccttccagccacaccccaaggcaagaacatgacccctgagagtgaggtgctggaatctttccccgaagaagactctgtactcagccattcgtccctgagctctccctcttccaccagctcgcccgaggggccgcctgctcccccaaagcagcacagcagtaccagtcccttccccacctccacgcccctccgcagaataactaagaaattcgtcaaaggctctacagagaagaacaagctcagactgcaaagagatcaggagcgtctgggcaagcagctcaagttacgtgcagaaagggaagaaaaggagaagctgaaagaggaggccaagcgggccaaggaggaggccaagaagaagaaggaggaagagaaggagcttaaggaaaaggagaggcgggagaagcgggagaaggatgagaaggagaaggcggagaagcagcggctcaaggaggagcggcgcaaggagagacaggaagccctggaggctaaacttgaggaaaaaaggaaaaaggaagaagagaaacggttaagagaagaagagaagcgcattaaagcagagaaggccgaaatcacgaggttcttccagaaaccaaagactccacaggcccccaagaccctggccggctcctgtgggaagtttgccccctttgaaattaaagagcacatggtcctggcccctcggcgtcggaccgctttccatccagacctctgcagtcagctggaccagctcctccagcagcagagcggcgagttctccttcttgaaagacctcaaaggccggcagcccctgaggtccggacccacgcacgtttccacccggaatgcagatatttttaacagtgatgtcgtcatcgtggagcgtgggaagggcgacggtgttcccgagaggaggaagtttggcaggatgaagctcctgcagttctgtgagaaccaccggcctgcctactggggtacctggaataagaagacggcactcatccgcgcgcgagacccctgggcccaggacacgaagctcctggactatgaggtggacagtgatgaggagtgggaagaagaggagcctggggagtccctgtcccacagtgagggggatgatgatgacgacatgggagaggatgaagatgaggacgatggtttctttgtgccccatgggtacctgtctgaggacgaaggtgtgacagaggagtgtgccgaccctgagaaccataaggtccgccagaaactgaaggccaaggagtgggacgagttcctggctaaggggaagcgctttcgcgtcctgcaacctgtgaagatcggctgcgtgtgggcggctgacagagactgcgcaggcgatgacctgaaggtactgcagcagttcgcagcctgcttcctggagaccctgccggcccaggaggagcagatacttgaaccgactcaattcctgtgtaaagagcactttgtcctgcttcacggacctccccaaagtgtgcagagttctatataggatgctggattagttcctttgatatttgtaaaaattcccccaagagccgcatatgaatctgccctttaataaagcattattgagattgctggcctattggggaagcctgcgggcacaggagcaggcgtggaatccaatacttgtaaatgaattgaagcgtcaggaccacccgcctggccacgtgcgcgggcccctggacctaacgaggcagtgtataaacttattctctagccctgaaaaaaaaaaaaaaaaaactcgg

SEQ ID NO: 96 - DNA, 2329 bp, Homo sapiens
  misc_feature: Incyte ID No 6946680CB1
catggggaacgcggtgtcgctggttcagatctcgcagagctcagagtcctgcatgcctcagtcctcgcctcgctcctcctcgcggaggattctgggaggtgacgtcgcgggtctcggtcgcggggcccgtttgcagagcccgcggcgccgggaggactttgttcttcttcagaagagaaaactgaagaaggaggaatggctgtggggctttgtaaagccatgtcccaggggttggtgaccttcagagatgtggcgctagacttttcccaagaagagtgggaatggctgaagccatctcagaaggatttatacagagatgtcatgttggagaactacaggaacttggtatggcttggactctccatttctaagcccaacatgatctccttactggagcaagggaaggaaccgtggatggtggagagaaagatgtcacagggtcactgtgcagactgggagtcttggtgtgaaattgaggaattatctccaaaatggttcattgatgaagatgaaatatcccaggagatggtaatggaaaggctagcaagtcatggccttgaatgctccagtttcagagaagcctggaaatataagggtgaatttgagctacatcagggaaatgcggagaggcatttcatgcaagtgacagctgttaaggaaatctctactgggaaaagagacaatgaatttagtaattctgggagaagcatacccctgaaatcagtatttttaacacaacagaaagttcctaccatacagcaagtacataaatttgatatttatgataaactcttcccccaaaattcagtcataattgaatataaaagactccatgctgagaaggaatctttgataggtaatgaatgtgaagaattcaaccagagtacgtaccttagtaaagatataggaattcctcctggggagaaaccttatgaaagtcatgatttttcaaagctcttaagtttccactcattatttactcaacatcagaccactcattttggaaaattaccccatggatacgatgaatgtggtgatgcctttagctgttactcattctttactcaacctcagagaattcacagtggagaaaaaccatatgcatgcaatgactgtggaaaagcctttagccacgacttctttctcagtgaacatcaaagaactcatattggggagaaaccttatgaatgtaaggaatgtaacaaagctttcagacagagtgctcaccttgctcaacatcagaggatccacactggagagaaaccgtttgcgtgcaatgaatgtgggaaggcctttagccgttatgccttccttgttgaacatcagagaattcacacaggtgagaaaccatatgaatgtaaagaatgtaataaagccttcagacagagtgctcaccttaatcaacatcagaggattcacactggagagaaaccctatgaatgtaatcagtgtggaaaagccttcagcagacgcatagcccttactctacatcaaagaattcacacaggagagaaacccttcaaatgtagtgaatgtgggaagacctttggctatcgctcacacctgaatcaacatcagagaattcataccggagaaaagccctatgaatgcatcaaatgtgggaagttttttaggactgactcacaacttaatcgacatcatagaattcacactggagagagaccatttgaatgcagtaaatgtgggaaagccttcagtgatgctttagttctaattcaccataagagaagtcatgcaggagagaaaccctatgaatgtaacaaatgtggaaaggccttcagttgtggctcatatcttaatcaacatcaaagaattcatactggagagaaaccctatgaatgtagtgaatgtgggaaggcttttcatcagatcttgtccctaagactacaccagagaattcacgctggagaaaaaccttataaatgtaacgaatgtgggaataattttagctgtgtctcagcccttagacgacatcagagaattcataatagagaaacgctctgattataacaagtataggaaagaaaacatgtggttaccactcaatccttattaaatattagtgagttctttttggttagtaattctttgaatgtagttcatatttcagttcatgagtatccgttatttgaagtagctcagtagtagacatctgttactttttttttttttccaaacaagagtctcgctctgttgcccaggcgggagtgcaggggtgacactgggcccacggaagccctgcaccctgggtcaagcaacctcaggccacagaccctgagaagcgggatacagggccgccacaaaccgg

SEQ ID NO: 97 - DNA, 1979 bp, Homo sapiens
  misc_feature: Incyte ID No 7001142CB1
cgagtgcagattccccgagccttcggggcaggaaggagatcttccaccagttctgttctgcaggtcgggagtgggctgaggagtggcgtgtgggtctccggaagctcgtcgcaggccatctgtgtgactccggtgcgagtggagtcatctgaggccactgctatttcccaagagaagagccaggaggaagaaagaatggctgttgggcttcttaaagccatgtaccaggagttggtgaccttcagagatgtggctgtagacttctcccaagaggaatgggattgtctggattcttctcaaagacatctgtacagtaatgtgatgctagagaactacaggatcttggtatcactgggactttgcttttccaaaccaagtgtgatattattgttggaacaaggaaaagcaccctggatggtgaagagagagctgacaaaaggcttgtgctcaggctgggagcctatatgtgagactgaagaattaaccccaaagcaggatttttatgaagaacatcaatcccagaagataatagaaacacttacaagctataaccttgaatactccagtttgagagaagagtggaaatgtgagggctattttgaaaggcaaccaggtaatcagaaggcgtgtttcaaggaagagataatcactcatgaagaacccctttttgatgagagagaacaagaatataaatcttggggaagttttcatcagaacccactgctttgtacacaaaagataatccccaaagaggagaaagtacataaacatgacacacaaaagagaagctttaaaaaaaatttaatggctattaagcccaagagtgtctgtgcagagaagaaacttttgaaatgtaatgactgtgaaaaagtcttcagccagagttcatcccttactcttcatcaaagaattcatactggagagaaaccctataaatgtatagagtgtggaaaagccttcagccagagatcaaatcttgttcaacatcagaggattcatactggagaaaaaccctatgaatgtaaggaatgtaggaaagccttcagtcagaatgcacacctagttcaacatctgcgagttcatactggagaaaaaccttacgaatgtaaggtatgtcgaaaagccttcagccagtttgcctaccttgctcaacatcagagagttcacacgggagagaaaccctatgaatgtatcgaatgtgggaaagcatttagcaacagatcatccattgctcaacaccagagagttcatacaggagagaaaccctatgaatgtaatgtctgtgggaaagcatttagccttcgtgcataccttactgtacatcagagaatacatactggagagagaccctatgaatgtaaggaatgtgggaaggcctttagccagaattcacaccttgctcaacatcagagaattcatactggagaaaaaccttataagtgtcaggaatgtaggaaagcattcagccagattgcctaccttgctcagcatcaaagagttcatactggagagaaaccctatgaatgtattgaatgtgggaaggcttttagcaatgactcgtcccttactcaacatcagcgagttcatactggagagaaaccttatgaatgtactgtttgtggaaaggcttttagttactgtggatcccttgcccaacatcagagaattcatactggagagagaccctatgaatgtaaggaatgcaaaaaaaccttcaggcagcatgcacaccttgctcatcaccagagaattcacattggggagtcactgtcaccacccaacccagtcaatcaccaagtcctatagatcctgagtcctaaatgtttctagaatttatactgttttttatctttaatgttgtcaccttggtctgattcatctcactctgattactaatatagctttcaaacaggttttcctgtatttatttttgctacctttaaatccatttcccacaatgcactcaaacggatcagcgg

SEQ ID NO: 98 - DNA, 2795 bp, Homo sapiens
  misc_feature: Incyte ID No 71158380CB1
ccgcggcgtcttctccacccacctgcgctgggcgctcggtgctcggctctgtagctaagagacgacctggaagttccgtggcagcctgtgtcccgcgggaacccgcattggcagcgggagccgtccggaggacctgggacaccgcggaagtcgggaaatggcctcagtggctttagaggatgtggctgtgaacttcacccgagaagagtgggctttgctgggtccttgtcagaagaatctctacaaagatgtgatgcaggaaaccatcaggaacctggattgtgtaggaatgaaatggaaagaccagaacattgaagatcaatatagatatcccaggaaaaatctaagatgtcgtatgttagagagatttgttgaaagtaaagatggaactcaatgtggagaaacatctagccagattcaagatagtattgtgaccaagaacactcttcctggagtaggtccatatgaaagccgtatgagtggagaagtcatcatgggtcattcatcccttaattgttacatcagagttggtgctgggcacaaaccatatgagtatcatgaatgtggagagaagccagatacgcataaacaacgtgggaaagccttcagttaccacaactcacttcaaacacatgagaggcttcacactggaaagaaaccatataattgtaaagaatgtgggaagtccttcagttctttgggaaaccttcaaagacacatggcagtgcagcgtggagatggaccttataaatgtaagttgtgtgggaaagcgtttttttggcccagtttattacatatgcatgagagaacgcacactggagagaaaccatatgaatgtaagcagtgttctaaagccttttctttttacagttcctatctaagacatgaaagaacacatactggggagaaactgtatgaatgtaaacagtgttctaaagccttccctgattacagttcttgtctaagacatgaaagaactcacactggaaagaaaccctatacatgtaaacaatgtgggaaagccttcagtgcttccacttcccttcgaagacacgaaacaactcacactgatgagaaaccctatgcatgtcagcaatgtgggaaagcgtttcatcatctgggaagctttcaaagacacatggtaatgcacacgagagatggacctcataagtgtaagatatgtggaaaaggctttgattgtcctagttcactgaaaagtcatgaaagaactcacactggagagaaactctatgaatgcaagcagtgtgggaaagcgttatctcatagctcaagctttcgaagacacatgacaatgcacactggagatggacctcacaaatgcaagatatgtgggaaagcctttgtttatcccagtgtatttcaaaggcatgaaaagactcacactgcagagaaaccctataaatgtaaacaatgtggcaaagcctaccgtatttccagttctcttcgaaggcatgaaacaactcatactggagagaaaccctataaatgcaaatgtgggaaagcctttattgatttctattcctttcaaaatcacaaaacaactcatgctggagagaagccatatgagtgtaaggaatgtgggaaagcattcagttgtttccaatacctttctcaacatagaaggactcacacaggagagaaaccttatgagtgtaacacatgtaagaaagccttcagtcattttggtaacttaaaagtacatgaaagaattcactctggagagaagccgtatgaatgtaaggaatgtgggaaagcattctcttggctcacttgctttctacgacatgaaagaattcacatgagagagaaaccctatgagtgtcaacaatgtggtaaagccttcactcattcccgttttcttcaaggacatgaaaaaactcatactggagagaacccgtatgaatgtaaggaatgtgggaaagcatttgcttctctcagttccttgcatagacataaaaagactcactggaaaaaaactcacactggagagaacccgtatggatgtaaggaatgtgggaaagcatttgcttctctcagttccttgcatagacataaaaagactcactagcattctctctaaatgtatggaatgtgggaaagcatttattaattttatttcatttcagatacttgtacgaaacacattggagatagaccctgtgaatgtaagcacttggtaaaaccttaagtaatttcagtttctttccagtacagtcatcccttgatacctgctgggtattggttccagcactccgtgagccatgtccagtcccttttataaaatgacatatttgtatgtaacttacccacatcctcttgtatactctcaattatgtctagattacttaaaatacctcatgcattgtaaaagctatgcaaatagttgttctattgtattgtttagggactcatgataaggaaaagagtctatgtattttcagtatagatgcagtaattgtcagcctatcaacatagtatagtcagccagaacattaaagtttctttgtttcaactctcaaaaaaaaaaaaaaaaaaaaaaaaaaaaaaaaaaaaaaaaaaaaaaaggggggggcgcgcgcctaatgaatgaactccgcaaagtttatagtcatgcaggcgatggatggtgaacattataatatgcactttttttataatggtggtaacacatgataatattattaatactggggacgaggataataaaaacaaagagatgtaggagggaacaacagtgttgtgttcaaaaaattaaaggctccttgagaactcct

SEQ ID NO: 99 - DNA, 4567 bp, Homo sapiens
  misc_feature: Incyte ID No 7503861CB1
agtttttcctttctccgcagctctgctcccctagcaacgctcgccacacccttgttttgagatcctctctaaggagcggagagtttaataggcaagaaggaagggagaagacagaaggaagacgctcccccgtacggagacagagggagggggggctccaaagccgaaagaggaggtccctacctgccacggataccagtcagcccttgccagcatccagccatgggggatatgaaaaccccagattttgatgaccttctggctgcctttgacatcccagaccccaccagccttgatgccaaggaggccatccagacacccagtgaggagaatgagagtcccctcaaacctccaggcatatgtatggatgaaagtgtgtccttgtctcactcaggatcagcccccgatgtgccggccgtgagtgtcattgtcaagaacaccagccgccaggagtcatttgaagcggagaaagaccacattactcccagtctcctacacaatggattccggggctcagatctgcctccagatccccacaactgtgggaaatttgattctacttttatgaatggagacagtgccaggagtttccctggcaaactggagcctcccaagtcagagccattacccaccttcaaccagttcagtccaatctccagcccagaacctgaggatcccatcaaagataacggatttgggataaagcccaaacactctgacagttatttcccaccccctcttgggtgcggggctgtgggaggcccagtcctggaggctctggctaagtttccggttccagagctgcatatgtttgatcatttttgtaagaaagaacccaagccagaacccctgcccttggggagccagcaggaacacgagcaaagtgggcagaacacagtggaacctcacaaggatccggatgccactcgattcttcggggaagctttggagttcaacagccatcctagcaacagtattggagagtccaaggggcttgcccgggagcttggtacctgctcatcagtcccccctaggcagcgtctaaagccagctcattccaagctgtcctcttgtgtggcagccttggtggccttgcaggccaaaagagtggctagtgtcactaaggaggatcagcctggccacacaaaggatctctcagggcccactaaagagagttctaaaggtagccccaaaatgcccaagtcaccaaagagtccccggagccctctggaggccactagaaaaagtatcaagccatcggacagccctcgtagcatctgcagtgacagcagcagcaaaggctcaccgtctgtggctgccagctccccaccagcaattcccaaagtgagaatcaaaaccattaagacatcatcaggggaaatcaaacggactgtcacaaggatcctgccagatcctgatgatccaagtaagtcccctgttgggtcacctctagggagcgccattgcagaggcccccagcgagatgccaggggatgaggtgcctgtggaagagcactttcctgaggcaggcacaaattcagggagcccccagggggccaggaaaggggacgagagcatgacaaaggccagtgactcgtcatctcccagctgcagttctgggccccgggtcccaaagggggctgccccaggctcacagacaggcaagaagcaacagagcacagcactgcaggcatccaccctggcccctgccaacctcctgcccaaagccgtgcacttggccaacctgaacctcgtcccccacagtgttgctgcatcagtgacagccaagtcttcagtgcaaagacggagccagccacagcttacacaaatgtcggtgcccctggtccaccaggtgaaaaaggctgccccactgattgtagaggtcttcaacaaggtccttcacagctccaaccccgtgcccctctatgcgccaaatctcagcccgcctgcggacagcaggatccacgtgccggccagtgggtactgctgcctggagtgtggagacgcatttgccttagagaagagcctgagccagcactatggccggcggagcgtccacattgaggtactgtgcacactgtgctccaagacgctgctgcccaaccagtgcagtttctgtgcccaccagcggattcatgcacacaagtccccctactgctgcccggagtgtggggtcctctgccgctctgcctacttccagacccatgtaaaggagaattgcctgcactatgcccgcaaggtgggctacaggtgcatccactgtggtgtcgtccacctgaccttggccttgctgaaaagccacatccaggagcgacactgccaggttttccacaaatgtgcattctgccccatggccttcaagactgccagcagcactgcagaccacagtgccacccagcaccccacccagccccacagaccctcccagctcatttataagtgctcctgtgaaatggtcttcaacaagaagaggcacattcagcagcatttttaccagaatgtcagcaagacgcaggtgggcgtcttcaagtgccctgagtgcccactcttgttcgtgcagaagccggagttgatgcaacacgtcaagagcacccacggtgttccccgaaatgtggacgagctgtcaagcctccagtcttcagcggacacatcctcaagccgccctggctctcgagttcccactgagccaccagccactagtgtggctgctcggagcagctccctgccttctggccgctggggtaggcctgaagcccaccgcagggtggaagccaggccgcggctgaggaacactggctggacctgccaggagtgccaggagtgggttccagatcgggagagctacgtgtcccacatgaaaaagagccacggtcggacattgaagcggtacccatgccggcagtgtgaacagtccttccacacccccaacagcctgcgcaaacacatccgcaacaaccatgacacagtaaagaagttctacacctgcgggtactgcacagaggacagccccagctttcctcggccctcccttctggagagccacatcagccttatgcatggcatcagaaaccctgatttgagccagacgtccaaagtgaaacctccgggtggacattcccctcaggtgaaccatctgaaaagaccagtcagtggagtgggggacgctccaggcaccagcaatggcgcaactgtctcttccaccaaaaggcacaagtccctttttcagtgcgcgaaatgtagttttgccacagactcggggctcgagtttcagagccacatacctcagcaccaggtggacagctccacagcccaatgtctcctctgtggtttgtgctacacctctgccagctccctcagccgccacctcttcattgtccacaaggtgagagaccaggaggaggaggaggaagaggaggcggcggcagcggagatggcagtggaggtggcagagccagaggagggctccggggaggaggtgcccatggagactagagagaatggactggaagaatgtgccggtgagcctttgtcagctgacccagaggcgaggagattgctgggcccggcccctgaggacgatggtggccacaatgatcacagtcaaccacaggcctctcaggaccaggacagccacacactgtcccctcaggtgtgaccggagactttgcagtgtgcatggtcaggggtggtgccgaagtgtcttccacctgccctgcggaccgtggaaaataaaaggctctgcccccagtgtgagtgtgaccggttgtaccctggagtagtgtctgccctgagctgccagtgctgggtatcccccagccccaggaaatgtggggtcggccaggaccctcacagctctgaatttgcttctgttatttatggcttttcgctgcttcttggtgccccatctcttgtctgtgtccttccaaccccaagctgcttatgtggcccaaccccactgctgtcaactaggcttgaaccccacagcggctgtgctcttctgggaggttcccgcttgctgccttcagccagggcgctcctcagagctctattttcctgcagacaccagctctccttcctgcctttagatcctgagaaggagggaaatgaggggtgctgacacagtccctctgggagagctctgcctagtctggtttggcgagggcccttgatcaccttgcccctcctccctgtcttctctgattcttttccctcaaaatagtcctgagaactaattgtcacactggctcatcatgtctctgtgggtggggtgggagaaacctctgctgcacacctctgtttggaacctgggcagagcaggaggtaaggcaaaggcaggcaggcaccaagaaccagaccccttgagaaggcgctgtgggtgggtctttgttctgctgttctgcctttcctgacaggtggggttggggcacacagacattggaatatttgtactgctctcgtgccatttgagaggctgctgccccaggcaggccagcccctactcctcttggctacactcatgttgctcagactatatttcaaataaaaaatcttctcaccatgcaaaaaaa

SEQ ID NO: 100 - DNA, 4507 bp, Homo sapiens
  misc_feature: Incyte ID No 7758395CB1
gtagagctggttcctgctcgccgcgggtgccgcgcgcgccggccggccgctgggcgctccgcgctcccagcctcgagttgtgcaatcctttgtagcacgccagagtcctcctcctccgctgttgcctctcgccctctctctttttttttttttcaagctgtgagctcaaccgatgagtcagagccgtgcaatcctgacactgcatcgcaggactgggggtgacacggagggaggcagagcgctcgcgaggcggacggcacgggtgcggggcgcgccgaggctcctgcatcgcaagcggggggtgacagcccgcgcgtcccgcccgggccctgccagcaaacttctcagcctcgggaggcgcgggctggcggaagccccgcgagcgccgcggggaggcgacggcgcctgtttgtttttaaaatcgggagtgcgtgcaggcggctggagtcccggaggcgaccgaaggcggcgacccgcggcggaagggggacagccgagcccggagcccggagcccgggcaagagctgggtgccagaaccctgtggagcatcatgaactgggaagagtagctgagccccagagcctctctggaagagaaaggaagagccagcagttctttctcccagtgtccgacctcactgtccagcgtcttcctctgcccctgctctgccctccctggctcctggactagagcccggcttccagcaggacgtttccccaggggatgggcgactgttgaaggggatctcaccgccagggctcagttggccacatcatgaacctccaggcccagcccaaggctcagaacaagcggaagcgttgcctcttcgggggccaggaaccagctcccaaggagcagccccctcccctgcagcccccccagcagtccatcagagtgaaggaggagcagtacctcgggcacgagggtccaggaggggcagtctccacctctcagcctgtggaactgccccctcctagcagcctggccctgctgaactctgtggtgtatgggcctgagcggacctcagcagccatgctgtcccagcaggtggcctcagtaaagtggcccaactctgtgatggctccagggcggggcccggagcgtggaggaggtgggggtgtcagtgacagcagctggcagcagcagccaggccagcctccaccccattcaacatggaactgccacagtctgtccctctacagtgcaaccaaggggagcccgcatcctggagtgggagtcccgacttactataaccaccctgaggcactgaagcgggagaaagcggggggcccacagctggaccgctatgtgcgaccaatgatgccacagaaggtgcagctggaggtagggcggccccaggcacccctgaattctttccacgcagccaagaaacccccaaaccagtcactgcccctgcaacccttccagctggcattcggccaccaggtgaaccggcaggtcttccggcagggcccaccgcccccaaacccggtggctgccttccctccacagaagcagcagcagcagcagcaaccacagcagcagcagcagcagcagcaggcagccctaccccagatgccgctctttgagaacttctattccatgccgcagcaaccctcgcagcaaccccaggactttggcctgcagccagctgggccactgggacagtcccacctggctcaccacagcatggcaccctaccccttcccccccaacccagatatgaacccagaactgcgcaaggcccttctgcaggactcagccccgcagccagcgctacctcaggtccagatccccttcccccgccgctcccgccgcctctctaaggagggtatcctgcctcccagcgccctggatggggctggcacccagcctgggcaggaggccactggcaacctgttcctacatcactggcccctgcagcagccgccacctggctccctggggcagccccatcctgaagctctgggattcccgctggagctgagggagtcgcagctactgcctgatggggagagactagcacccaatggccgggagcgagaggctcctgccatgggcagcgaggagggcatgagggcagtgagcacaggggactgtgggcaggtgctacggggcggagtgatccagagcacgcgacggaggcgccgggcatcccaggaggccaatttgctgaccctggcccagaaggctgtggagctggcctcactgcagaatgcaaaggatggcagtggttctgaagagaagcggaaaagtgtattggcctcaactaccaagtgtggggtggagttttctgagccttccttagccaccaagcgagcacgagaagacagtgggatggtacccctcatcatcccagtgtctgtgcctgtgcgaactgtggacccaactgaggcagcccaggctggaggtcttgatgaggacgggaagggtcctgaacagaaccctgctgagcacaagccatcagtcatcgtcacccgcaggcggtccacccgaatccccgggacagatgctcaagctcaggcagaggacatgaatgtcaagttggagggggagccttccgtgcggaaaccaaagcagcggcccaggcccgagcccctcatcatccccaccaaggcgggcactttcatcgcccctcccgtctactccaacatcaccccataccagagccacctgcgctctcccgtgcgcctagctgaccacccctctgagcggagctttgagctacctccctacacgccgccccccatcctcagccctgtgcgggaaggctctggcctctacttcaatgccatcatatcaaccagcaccatccctgcccctcctcccatcacgcctaagagtgcccatcgcacgctgctccggactaacagtgctgaagtaaccccgcctgtcctctctgtgatgggggaggccaccccagtgagcatcgagccacggatcaacgtgggctcccggttccaggcagaaatccccttgatgagggaccgtgccctggcagctgcagatccccacaaggctgacttggtgtggcagccatgggaggacctagagagcagccgggagaagcagaggcaagtggaagacctgctgacagccgcctgctccagcattttccctggtgctggcaccaaccaggagctggccctgcactgtctgcacgaatccagaggagacatcctggaaacgctgaataagctgctgctgaagaagcccctgcggccccacaaccatccgctggcaacttatcactacacaggctctgaccagtggaagatggccgagaggaagctgttcaacaaaggcattgccatctacaagaaggatttcttcctggtgcagaagctgatccagaccaagaccgtggcccagtgcgtggagttctactacacctacaagaagcaggtgaaaatcggccgcaatgggactctaacctttggggatgtggatacgagcgatgagaagtcggcccaggaagaggttgaagtggatattaagacttcccaaaagttcccaagggtgcctcttcccagaagagagtccccaagtgaagagaggctggagcccaagagggaggtgaaggagcccaggaaggagggggaggaggaggtgccagagatccaagagaaggaggagcaggaagaggggcgagagcgcagcaggcgggcagcggcagtcaaagccacgcagacactacaggccaatgagtcggccagtgacatcctcatcctccggagccacgagtccaacgcccctgggtctgccggtggccaggcctcggagaagccaagggaagggacagggaagtcacgaagggcactacctttttcagagaagaagaaaaaaacagagacattcagtaagacccagaatcaggagaacactttcccctgtaaaaaatgtggcagggtgttttacaaggtgaagagccgcagtgcgcatatgaagagccacgcagagcaggagaagaaggctgcagcgctgaggctgaaggagaaagaggccgctgctgccgccgccgccgcccaccagcaggccctgcgggaggagagcggtgcgggcgacaagggctgagcgcgggagccaggctggcccagtcctgggcctcggcccttcccgcaccgccgccagcgcccgcagacacctggcatctcaagagggagtgaggagaggattgcagggacttttccctgcgaaacaaatgagacaatgacataaacggctcttttatttatgaaggccctgggagcagcgttaagggctccaggatccagctctctttgcatttggtctgtcggaagctgtcctcgtgctttcctggaccgggagagtcccggtcccctcgggagggactccaccgcctctcacactccgatttctgctgctctgctgccccgcagtcttttccctttatttgcttccccctcctcccctcggcctccaggaagccaccgtggccggccaagcacaagctcacccactttggagcagcatttctcccccccc

SEQ ID NO: 101 - DNA, 4862 bp, Homo sapiens
  misc_feature: Incyte ID No 71039312CB1
agagaattccataatcacagcgggcgagacggagactgggagcggaaaatccgggatccggcaactttgggcagcgcatgcgcgcccgcgacgctccatcccaaacacacacacatttttcccccggacttggaaagtcacccaaagccgccccaagtgcaggcaaacagaccttgctgactgaaggcaaagactcctccatcccacctgccctcccagctgccacggccatcgacccctcctttacaaggggctcctgcaactccccagcagcacgtgttggaatggacttcggaccctggcctcttgggatgcaaaggatgaagtgacacccccagctacatccgaggaggttctaggacctgctacgagagtttgggaccaaggagaagagaatggatcttggaacagctgagggcacccggtgcacggacccgcctgcaggcaagcccgccatggcgcccaaacgcaagggtggcctgaagctgaacgccatctgcgccaagctgagccgccaggtggtggtggagaagcgagctgacgccggctcccacacggagggcagcccatcgcagccccgggaccaagagcgcagtggccctgagtctggggcagcccgggccccccgcagcgaggaagacaagagacgggcagtgatcgagaagtgggtgaacggggagtacagcgaggagccggcacccacacccgtgttggggcggattgcccgcgagggcctggagctgcctcccgagggtgtctacatggtgcagccccaggggtgcagcgatgaggaagaccacgcggaggagccctccaaggacggcggtgccctggaggagaaggattcggacggggcagcctccaaggaggacagcggccccagcaccaggcaggcttcaggagaggcctcctcgctgcgggactacgcggcctccaccatgaccgagttcctcggcatgtttggctatgatgaccagaacacgcgggacgagctggccaggaagatcagctttgagaagctgcacgcgggctccaccccggaggcagccacctcctccatgctgcccacctccgaggataccctcagcaagcgggcgcggttctctaagtatgaggagtacatccgcaagctcaaggctggcgagcagctctcctggccggcccccagcaccaagaccgaggagcgggtgggcaaggaggtggtgggcaccctgcccggcctgcggctgcccagcagcacggcccacctggagaccaaggccaccatcctgcccctgccgtcgcacagcagtgtccagatgcagaacctggtagcccgggcctccaagtacgacttcttcatccaaaaactgaagaccggcgagaatctgcggccccagaacgggagcacctacaagaagccatccaagtacgacctggagaatgtcaagtacctgcacctcttcaaacccggggagggcagccccgacatgggcggggccatcgccttcaagacaggcaaggtggggcgcccttccaagtacgacgtccggggcatccagaagccaggccccgccaaggttccgcccacccccagcctggctcccgcacccctcgccagcgtgcccagtgcccccagcgcccccgggccagggccagagcctcctgcctccctgtccttcaacactcccgagtacctgaagtcaaccttctccaaaacagactccatcaccacggggaccgtctccactgtcaagaacggactgcccacagataaaccagccgtcactgaagatgtaaacatttaccagaaatatattgccaggttctcgggcagccagcactgtggccacatccactgtgcctaccagtaccgcgagcactaccactgccttgaccctgagtgtaactaccagaggttcacgagtaagcaggacgtgatccgccactacaacatgcacaagaagcgcgacaactccctgcagcacggcttcatgcgtttcagcccgctggacgactgcagcgtctactaccacggctgccacctcaatgggaagagcacccactatcactgcatgcaggtgggctgtaacaaggtgtacacgagcacgtctgacgtgatgacccacgagaacttccacaagaagaatacccagctcattaacgacggcttccagcgcttccgagccaccgaagactgtggcacagccgactgccagttctacggacagaagaccacgcacttccactgcaggcgccccggctgcacattcactttcaagaacaagtgtgacatcgagaagcacaagagctaccacatcaaggacgatgcctacgccaaggacggcttcaagaagttctacaagtacgaggagtgcaagtacgagggctgcgtgtacagcaaggctaccaaccacttccactgcatccgcgccggctgcggcttcaccttcacctccaccagccagatgacctctcacaagcgcaagcatgagcgccggcacatccgctcctcgggcgcgctggggctgccgccctcgctgctgggcgccaaggacacggagcacgatgagtccagcaacgacgaccttgttgacttctccgccctgagcagcaagaactccagcctgagcgcctcccctaccagccagcagtcctctgcgtccctggctgccgccactgccgccaccgaggctgggcccagtgccaccaaacctcccaacagcaagatctcggggctgctgccccagggcctgcctggctcaatccccctggccctggccctctccaactcgggcctgcccacccccacgccctacttccccatactggctggccgtgggagcacctccctgcctgtgggcacccccagcctcctgggtgccgtgtcgtctgggtcagcagcctcagccacccctgacacacccacgctggtcgcctcgggagctggagactcagcccccgtggctgccgcctctgtcccggcaccacccgcctccatcatggagaggatctctgcaagcaagggcctcatctcgcccatgatggccaggctggctgcagctgccctcaagccctctgccacctttgacccaggtgagcaggctgggtcctgcccagaaagcaggcaccttctggactggggtggccacctggcctcaggctgcagagcagagtcggggggtgtttggaagaagtgtccgtgtggcccctgcttgccagcaccccctctttgcctgatcttcgcccccatcctgttggtttctatgtggggcccctggttctggctccccacagtaggggggccatgcttctgtttccccatctgtgagctgggggcgatggtccctgcctcctcggctgccctgcatccctggggccactgcaggtcccccctccctgtctgctcaccctcccacatcccaagtccaccctgataagagcaggcccagggcccctggcgtgccgagcgtttcctccagctgacctggagcagggcctgcgtccagctcagcggtagtggggctgcacctcacacctggacacctccaggcccagggacgcccttcccaggctccctcctccatgagtgccattgcagtaggactttccaggccttggaagccttctattctgcagtgtttgacttggcagccactggccacgtgtgactgttggtgcttgaagctgggtctgtgattgggaagcgggattgtcactttaaacaaccacacttggccagggactgccacgtcagacagaaaagggccagagccccggagttgcatcttccttcttcacgctggttcactgaggggcgcagttctctttcctgccttcctataatttcataaatatccattttaaaaaaatttccctccatttttctctggattggggtgagggtgttttctgtttacctcactcctctcccagtttccctgctctgggtagttcctatttgttgaatccttgaggagagttccgttcacatctgggatgtggctcatttcacttattcattcaacaaccactgatgcttgctgagagccaggctccaagccgggtgctggggacacagcaggcgggacgccagacagagccccacgccgtggagccagcagacggtcagggatggtctgtcccgggcacatggctaggaagtggtgaaccaaggcgctgcccgcaggagtggggctcctgggcctagatgccctgttcccattcgaaaccctctcagaaccagccaagactgagggtccctgtggcccagtctggggctctgcccacaccttgcagaggctgccagtgcttggcaggggagctgggccatgttttcctgggaccttgggcaggtgactttggagagctgctggaatgtgaagtcccaaggtctgaaaactctcacaggcccaataggacgctcatgcccggtggccgcgtggagggaccgcctcctggtccaggcatgggtggggcctcgctggccacgcccttccagccaggtgcctgatggatggggcagggcccactccagggccacgtgggctcggtggccagactgtcaagtactttgttttcattagaaaagtctccttctcccaaaagaaagatcaagcacaggggagggggccgttttattttttaataaaatgccacttgctgtttctgaagcagcactctgtcagctacagcaccttggggcaaattcatatttccgtcactgggggattgtaggggaaagctcgctggatggccctgtcagcaacttcttttacccctgctctgctggcctcctgcggcctcacccgcccagctcccttccggacccagctcggcccagcctggcctcgtgtccctgccccgagagaaacaccatggtc

SEQ ID NO: 102 - DNA, 3011 bp, Homo sapiens
  misc_feature: Incyte ID No 7291318CB1
tccacaggcatgcaccaccaagcctacctagatttgtatttttttgtagaggtggagtctcactatgttgctgcctagaatggtcttaaactcctgggctcaagcgatctcccaccttagcctcccatggtgctgggattacaggtctgagccaccgtacccagccgccacaggggttttgcctgggtctggcttgtaagggtgatgtcagagcatgatgctgggcctaccttccaaagaggctgcacttcttttttcaggaatggacaatcagaccgttctggctgtccagtcattattggatggccaaggagcagtccctgatccgacaggccagagtgtcaatgcgccccctgctatccagccattggatgacgaggatgtatttctctgcgggaagtgtaagaagcaattcaactcgctgccagcgtttatgacccacaagcgggaacagtgccaggggaatgcccccgccctggccacagtctcactggccaccaacagcatctacccaccttcggcagcacccacagcggtccagcaggccccaactcctgccaatcgccagatctccacatacatcacagtgcccccgtccccactgatccagaccctggtgcaggggaacatcttggtgagcgatgatgtgctcatgtctgccatgtcagccttcacatccctggaccagcccatgccccagggccccccacctgtgcaggtgccaaaccagtgtgtggagcctccagtatatcccacccccacagtgtacagccctggcaaacagggattcaaacccaaaggaccaaaccccgccgcccccatgaccagcgccaccgggggcacggtggccacctttgactctccagcaacgctgaagacccgacgagctaaagctgcagggaagccaaaggctcagaaactcaagtgctcatactgtgacaagtcattcaccaaaaactttgacctgcagcagcacatccgaagccacaccggtgagaagcccttccagtgcattgcatgtggccgtgcctttgcccagaagtctaatgttaagaaacacatgcagacccacaaggtgtggcctccaggacacagtggtggcaccgtgtctcgaaactctgtgaccgtacaggtcatggccctgaaccccagcaggcaggaggacgaggaaagcacagggttgggccagcccctgccgggtgcgccacagccccaggccttgtccacagctggtgaggaagagggggacaagccggagtccaagcaggtggtcctcatcgacagctcctacctgtgccaattctgccccagcaaattcagcacctacttccagctcaagtctcacatgacccagcataagaatgagcaggtatacaagtgtgtggtcaaaagctgtgcccagacgttcccaaagctcgacacatttctggagcacatcaagagccaccaggaggagctgagctaccgctgccacctctgcggcaaggacttcccctcgctgtacgacctgggcgtgcaccagtactcccacagcctcctgccacagcacagccccaagaaggacaatgccgtctacaagtgtgtcaaatgtgtcaacaaatactccacccctgaggccctggagcaccacctgcagaccgccactcacaacttcccctgcccacactgccagaaggtgtttccttgtgaacgctacctgcggcgtcatctgcccacccacggcagcgggggcaggttcaagtgccaagtgtgcaagaagttcttccggcgggagcattatctcaaactgcatgctcacatccactcgggtgagaagccctacaaatgctcagtgtgcgagtctgcgttcaaccgcaaggacaaactgaagagacacatgttgatccacgagcccttcaagaaatacaaatgccctttctcgacgcacacaggctgcagtaaggagttcaaccggccggacaagctgaaggcccacatcctctcccagtctggcatgaagctccacaaatgcgccctgtgcagcaagtccttcagccgccgtgcccacctcgccgagcatcagcgcgcccacacgggcaactacaagttccgctgtgctggctgcgccaagggcttttcccgccacaaatacctcaaagatcaccgctgtcgtctcggcccccaaaaggacaaggacctgcaaacccggcggcccccccagaggagggcagccccccgcagttgcggcagtggtgggcgcaaggtgctgacccccttgcctgacccgctggggctggaggagctgaaggacacaggggctgggctggtgcccgaggctgtccccggcaagccgcccttcgcagagccggacgcggtgctgtccatcgttgtgggtggtgcggtgggcgcggaaactgagctggtggtacctggacacgctgaggggctgggctccaacctggctctggcggagctgcaggctggggccgagggcccatgtgccatgctcgctgtgcccgtctacatccaggcctccgagtgacggacctgaggtgtctgtttcctgggcaggcctgatgctcctgtttgggtccagggcccctgggggcagaccggtgatccttaccagtggaagcgagccatcgagccattggcagaaatcctgctgaatgtcattcagaaacctcagcccatggtcgccctcctgtgcccctctcctgccggaaagccctgcaacattctagggttgggggcagggccatccacggtttctgggcagagccatggtggcaggagagagatggctgaagcctgagcagcccagagtcccgctggtctaggctggtggtcggggcccctgggagaggagacagggcattcctccccactctgtctccaggctgcctctgggtagcctctagtctgctgttcttcaggaggcctgccataaactcttcggagtttacgtgttgcaccttttcacagcggttccccacagggggatccactagtttagaacgccggccccgtgc

SEQ ID NO: 103 - DNA, 3092 bp, Homo sapiens
  misc_feature: Incyte ID No 2638619CB1
atgtccagcagcaggttttgggccggccgagctaaccctgcctctcttccttcgcaggcctcctcgctggggaggcagagtcctcgcgtggtctcctgcctcgagcacagcctgtgcccaggggagccgggcttgcagacaacagcagtggtgtccatgggctctggagaccatcagttcaacctcgcagagatcctgtcacagaactacagtgttaggggggagtgcgaggaggcctcgaggtgcccagacaagcccaaggaggagctggagaaggacttcatctcccagagcaacgacatgccctttgatgagctgcttgcgctctatggctacgaggcgtcagaccccatttcagaccgggagagtgagggtggtgacgtggccccgaacctcccagacatgaccctggacaaagaacaaatagcgaaggatttgctttcaggggaagaagaggaagagacgcaatcatctgctgacgacctcaccccgtccgtgacctcccacgaggcctccgacctcttccctaaccggagtggatctcgtttcctggctgatgaagacagagagcctggctcttctgcctcctccgacaccgaggaggactctcttcctgccaacaaatgtaagaaggagatcatggtgggacctcagttccaagctgacctcagcaacctgcacttgaaccggcactgtgagaagatctacgagaacgaagaccagctgctctgggacccagcgtcctccctgagaggggaggtggaggagttcctgtacagggcggtgaagcggcgttggcacgagatggccgggcctcagctcccagagggagaagccgtgaaagacagtgagcaggcgctgtacgagttggtgaaatgcaacttcaatgtggaggaggccctgcgaaggctgcggttcaacgtgaaggtgatccgagatgggctctgtgcttggagtgaagaggagtgcaggaactttgagcacggcttccgtgtgcatggaaagaactttcacctgatccaggccaacaaggtgcgcacacggtcagtgggcgagtgtgtcgagtactactacctgtggaagaagtcggagcgctacgactacttcgcccagcagacgcggctgggccggaggaagtacgtcccgtccggaaccacggacgcagaccaggacctggatggcagcgaccccgatggccccggccgtccgcgcccggagcaagacaccctgactgggatgcgcacagatccactgagcgtggatggcacggccggtggtctcgatgagcccggagtggcctctgatggactcccgtcctcggagccagggccgtgttccttccagcagctggatgagtcccccgctgtacccctgtcccatcggcccccagccctggccgacccagcctcataccagccagctgtcactgctccggagccagacgccagcccaaggctggccgtggacttcgccctgcccaaggagctgcccctcatctccagccatgtggacctcagcggggatccggaggagactgtggccccagcacaggtggctttgtcggtcaccgagtttggactcatcggcattggggacgtgaaccccttcctggccgcccaccccacgtgcccggcccccgggctacactcggagcccctgtcacactgtaacgtgatgacctgctgactcctggccgcgggcggcgtatgcggcccagactggacttagcgctgccgctgggcccgcctctgtcagtcttcctgaccccttccccaccccccgggccttggggtagcacctccttctgcttcagaacacgtcaggactggggtgaggtggctgggccgtgagcccttgcccctgtccacacagaatggacccacggccccacccagcgccgtcagcgcccggcactgccacccgggtccgggccgctgcctgcacgtgggatccgtcgggcagccggggacagaagagaccccgccgttgggacgcagggcagagccggccacctagtcccttccagccagcagaggcgagggaaggcgtcactgccccggcggggagacgggcaggacgccctgccccgcaccagcagcctccgccggggcgccctcagctccctgcttggctctgtctctccacacccggcagggccgcgggctgccccagccctgggggtcgtgggcagctgctactcagtgccaaccccgtggggcacagagccatatacctcgctgtccggcccccaccccagcctcgccttcccaccccatcgtctccacttcaggaaaagccgcactttacacccccacctgcctcttccccctccatccctgctccccgatcctgagcggttggggtggggtccctcagcaaccccaggcgtgggtttgaggagacaggtgatttacatcccctttgctgtcctcccccggtaccaaggcagggagcctccggaggagccggccctgctggccacgcaggggccagactccagcctgtttccccagccctgcaggtcttccttctgtgggaagcttcctagcaagatggcttggagtcctggtccccctcctccctggccctctcgttcgtttctgtttctgtttacacgttggagtggggtcctccgtgggcggcggcgcgccctgccccgggtgtcgtccggcctcttgtgctcgagcccctttccgagttggactcgaccatccctcaccccaccaaggaccacactgtgaagtgataactgccttgaacccccctttgctgttttatttattaaacttgatttgaagcccggaaaaaaaaaaaaaaaaagcggccctcgggatctagaaactagacgagagggaagcgcacgatagctgcgcggagagagagcgaagagcaggagggaggaacaagggcgacccaagacacccagagagggacagagaaccagggcagtctggcagtaccgccgccagtgaggaggcacgcagccttcgagaatgtgagctctacgtccagaagcatacattcaagcgctgctcaagattctatggtgcagttgtgcctgttcggactgagaaccatggtttcctcaggga

SEQ ID NO: 104 - DNA, 879 bp, Homo sapiens
  misc_feature: Incyte ID No 2810014CB1
gtagatttgttcaatcattcagccaatatttattgagtactactgagtggtaggtcagtttttggccctggatttacaaccacgggaataaaaacagagaagatgcctaccttcatggaatttacagtcttattttgttttatagaaatcatgtgtcctagtaatcaaatggggaaaaacagatttgtcttgaaagcagacaaagagaaatggaaaaatcaattacccctgtattactgtgtggagaaatgaaggcattcgtttggtttggcaggtcatctaagcagagtgctgcaaaagaaaaggatttgttgccttctcccgctgggcctgttccttcaaaagatccaaaaacagagcatggctctcggaagaggactattagtcagtcttcttccttaaagtcaagcagtaacagcaacaaggagacgagtggcagcagcaaaaacagttcctccacatcaaagcagaagaagaccgaagggaagacttccagtagctccaaggaggttaaggtaaagtgttgggggcctggggcttttgaaaatcattcaacttgccatgtgacttttccagggtgacactgtgctttgaaattattgtaacatctcagtaaatatataggcctatgtcttttaccctctatatgtaataatcctgataaatgaatacagtgatataagactgtgaaggcggtaagtcagctggtcacatacatattgaaagaaaaacccttgggtacagtggctcatacctgtagtcccagctatttgggaggctaagatggaaggatcatttgaggaatttgagtccagcctgggcaatgtggtgagaccctgtctgtaaaagaactttaaaatttaaaaaaaaaaaaaaagg

SEQ ID NO: 105 - DNA, 4574 bp, Homo sapiens
  misc_feature: Incyte ID No 3457155CB1
atgagcaccgccgccttccacatctccagcctcctggagaagatgacgtccagcgacaaggacttcaggttcatggccaccagcgacctgatgtcggagttgcagaaggactccatccagctggacgaggacagcgagcgcaaggtggtgaagatgctgctccggctcctggaggacaagaacggtgaggtgcagaacctggctgtcaagtgcctgggtcctctggtggtcaaagtgaaggagtaccaggtggagaccattgtggacaccctgtgcaccaacatgcggtcagacaaggagcagctgcgagacattgccggcattggcctcaagaccgtcctctcggagctccctcctgcagccacaggctccgggctggccaccaacgtgtgccggaagatcacaggccagctcaccagtgccattgcccagcaggaggatgtggctgtgcagctggaagccctggacatcctctctgacatgctgagcaggctgggtgtcccgctgggcgccttccacgccagcctcctgcactgtctgctgccacagctgagcagcccgcgcctggcggtgcgcaagcgggcggtcggagcgcttggccacctggcggccgcctgcagcaccgacctcttcgtcgagctcgctgaccacctactggaccggctgcccggcccgcgggtgcccaccagcccgactgccatccgcaccctgatccaatgtttgggcagcgtcggccgccaggccggccaccgcctcggggctcacctggaccgcctggtgcccctggtggaggatttctgcaacctggatgatgatgagctccgggagtcctgcctccaggcttttgaggccttcttgaggaagtgccccaaggaaatgggtcctcacgtgcccaacgtgaccagcctctgcctccaatacataaaacacgaccccaactacaactacgacagtgatgaggatgaggagcagatggagacagaggatagtgaattcagtgagcaagagagtgaagacgagtacagcgatgacgatgacatgagctggaaggtgcgccgggcagctgccaagtgcatcgcagccttgatcagctcgcggcctgacctgctgcccgatttccactgcaccctggcacctgtgctcatccgccgcttcaaagaacgcgaggagaacgtcaaggctgacgtcttcactgcttacatcgtgctgctgcggcaaacacagcccccgaagggatggctggaggccatggaggaacccacccagaccggcagcaacctccatatgctacgtggacaggtgccccttgtggtcaaggccctgcagcggcagcttaaagatcggagcgtcagagcccgccagggatgcttcagcctcctcaccgagctggcgggtgtcctcccaggcagcctggccgagcatatgcctgtgctggtatcaggcatcatcttctcgctggccgaccgctccagctcctccaccatccggatggatgccctggccttcttgcaggggctgctgggcaccgaaccagctgaggccttccacccacacttgcctatcctcctgccacctgtgatggcctgtgtggctgactctttctacaagattgcagccgaggccctggtggtgctgcaggagctggtgcgggccctgtggccgctgcacaggcctcggatgctggatcctgagccatatgttggagagatgtctgctgtcaccctggcgcgacttcgtgccactgacctggaccaggaggtgaaggagcgggccatttcctgcatgggccaccttgtaggccacctgggtgaccggcttggggatgacctggagcccacgttactgctcctcctggaccgcctgcggaatgagatcacccggctgcccgccatcaaggcgcttacgctggtggccgtatccccactacagcttgacctacagcccatcctggccgaggcactgcacattctggcctcattcctgcggaagaaccagcgggctttgcgactggccacactggcagccctggacgccctggcccagagccagggcctcagcctcccaccgtctgccgtgcaggccgtgctggctgagctgcctgccctggtcaacgagagcgacatgcatgtggcccagctggctgtggacttccttgccacagtgacccaggcccagccagcctctttggtggaggtcagtggccctgtgctctcagagctgctgcggctgctgcgttcgcccctgttgccagccggggttctggcagctgctgaaggcttcctgcaggccctggtagggacccgtcccccgtgtgtggactatgccaaactcatcagcctgctcactgcgcctgtttatgagcaggctgtggatggtgggcctggcctgcacaagcaggtgttccactcattggcccggtgtgtggcagccctctcagctgcctgtccccaagaggcggcaagcacagccagtcgcctggtctgcgatgccaggtcgccccactccagcacgggggtcaaggtcctggcattcttgtcgctggctgaggtgggtcaggtggctgggccaggcccccagcgggagctgaaggcggtgctcctggaagctttggggtcacccagtgaggatgtgagggctgcagcctcgtatgcactgggccgtgtgggtgctggcagcctgcccgacttcctgcccttcctgctggagcagatcgaggctgagccccgacgacagtacctgctgctgcactcactcagggaggccctgggggccgcccagcctgacagcctgaagccctacgccgaggacatctgggccttgctgttccagcgctgcgagggtgctgaggagggcacccggggggtggtggccgagtgcattgggaagctggtccttgtgaacccttcgttccttctgccccgcttgcggaagcagcttgctgcaggtcggccacacacccggagcaccgtcatcacagcggtcaagttccttatctcggaccagccccatcccattgaccccctcctgaagagcttcatcggagagttcatggagagcctgcaggacccagacctgaacgtgcgccgtgcgactctggctttcttcaactcagctgtgcacaacaagccctcgctagtccgggacctgctggatgacatcctgcccctcctctaccaggagacaaagatccggcgggacctcatccgagaggtggagatggggccctttaaacatacagtggacgatgggctggacgtgcggaaggcggcctttgaatgcatgtattcactgcttgagagctgcctgggccagctggatatctgtgagttcctgaaccatgtggaggacgggctgaaggaccactacgacatccggatgctgaccttcatcatggttgcccggctggccaccctgtgtcctgcacctgtcctgcagagggtggaccgactcattgagccactaagggccacctgcactgccaaggtcaaagctggttctgtgaagcaggagtttgaaaagcaagatgaactgaagcgctctgcaatgagggcagtggctgccctgctgaccatccccgaggtggggaaaagccccatcatggccgacttctcttcccaaatcagatccaaccctgaacttgctgccctctttgaaagcatccagaaggattccacttcagcccccagcacagactcaatggagctcagctagtcccctcagcaccaaggtgggccctcgcttaagagaaaggagcccacccaagtccgaggcctccccatcccaccatcgcaggtctctacttttgcccttccaccatctcactgggggccctgtcgctcctggtcagggcttacagtgccttctccagggacccaactcaaaggcccccagcccaagctgtgaggctgccaacagttgggccccttccttaactcaggacagtcatccaaagaaatagggtgaggaagttttccagtgacttcacactgtacccctccatagtctgtctggttccttcagagggtgtctctgcctcacaaactagtagtatttagaaataggctgtgctgtcagctgtaaaagatcaggaggcagcagacaccactctggtttcttcactgcattcagcaatgcctgaagttagtgctcaggccgggcatctcaaaagaaaagatacttgagttattcacattttaaaattcaaaacggttcatttttaagtggcagtgatgaatcagaaatttggaagatgatacgggtttcttttttccagggaggaggaatgggttgggtagggaactggacaggcttggacctcatgtttcatttctaatttcaaaatacttattagcaaattgggcaacaatgggcatcttccatgccaccacccaggcataaccagttggtttgtttccttctgaggaaggtttcaaatgtgtctagtgttcagtattgaggacaaagaaatacaagtggcaggcccaagtattttctgtgatatcccaggttaataaagattagattctaagttacttctttcctcaaaaaaaaaaaaaaaaaaaaaaaaaattggt

SEQ ID NO: 106 - DNA, 1483 bp, Homo sapiens
  misc_feature: Incyte ID No 7435171CB1
atgccggaacccgggccggacgctgccggcaccgccagcgcacagccccaaccgccgccgccccccccacccgctcccaaggagtccccgttctccatcaagaacctgctcaacggagaccaccaccggccgccccctaagcctcagccgcccccacggacgctcttcgcgccagcctcggctgccgccgccgccgccgctgccgctgccgcggcggccaagggggccctggagggcgccgcgggcttcgcgctctcgcaggtgggcgacctggctttccctcgctttgagatcccggcgcagaggtttgccctgcccgcgcactacctggagcgctccccagcctggtggtacccctacaccctgacccccgccggcggccacctcccgcgacctgaagcctcggagaaggccttgctgagagactcctcccccgcctccggcacagaccgcgactctccggagccactgctcaaggccgaccccgatcacaaggagctggactccaagagcccggacgagatcattctggaggagagcgactccgaggaaagcaaaaaggaaggcgaagcggcgccaggcgcggccggggcgagcgtaggggcggcggcggccactccgggcgcagaagactggaagaagggcgctgaaagtccagagaagaagccggcgtgccgcaagaagaagacgcgcacagtcttctcgcgcagccaggtcttccagctcgagtccaccttcgacatgaagcgctatctgagcagctcggagcgagccggcctggccgcgtccctgcacctcaccgagacgcaggtcaagatctggttccagaaccgccgcaacaagtggaagcggcagctggcggcggagctggaggcggccaacctgagccatgccgcggcgcagcgcatcgtgcgggtgcccatcctctaccacgagaactcggcggccgagggcgcggcggctgcagccgcgggggccccggtgccagtcagccagccgctgctcaccttcccgcaccccgtctactactcgcacccggtggtctcttccgtgccgctgctacggccggtctgaggccccagaggggtgggggagggagcgcccggctccttgtcggaccccggaggagactgggccgggccgagggcgccgagaagtccagcggcttcaggaactggggcttgggcgcgcagcctctgcttcccctcccccagtcggtagcatttgtaagtatttgcaatgcattttcgtgcaattcatccctaatggattggaggcgcttcccctcttactttggttttggcttatattaagagaaagcaggaacaagacaaaatttccgggtcagagatttcggccgatagtttttggtaaaatgtgcagcctcccttccaaatttccattgcgcggtggcttttggtttatttttatagaaggacaataagcgcaaaactagatccctctagtttattcttttctgctt

SEQ ID NO: 107 - DNA, 994 bp, Homo sapiens
  misc_feature: Incyte ID No 7499936CB1
gaattccggcgccgggggccgcccgcccgccgcccgctgcctgcgccgccggccgggcatgagttagtcgcagacatggacaccaaacatttcctgccgctcggctggaatgagctgctcatcgcctccttctcccaccgctccatcgccgtgaaggacgggatcctcctggccaccgggctgcacgtccaccggaacagcgcccacagcgcaggggtgggcgccatctttgacagggtgctgacggagcttgtgtccaagatgcgggacatgcagatggacaagacggagctgggctgcctgcgcgccatcgtcctctttaaccctgactccaaggggctctcgaacccggccgaggtggaggcgctgagggagaaggtctatgcgtccttggaggcctactgcaagcacaagtacccagagcagccgggaaggttcgctaagctcttgctccgcctgccggctctgcgctccatcgggctcaaatgcctggaacatctcttcttcttcaagctcatcggggacacacccattgacaccttccttatggagatgctggaggcgccgcaccaaatgacttaggcctgcgggcccatcctttgtgcccacccgttctggccaccctgcctggacgccagctgttcttctcagcctgagccctgtccctgcccttctctgcctggcctgtttggactttggggcacagcctgtcactgctctgcctaagagatgtgttgtcaccctccttatttctgttactacttgtctgtggcccagggcagtggctttcctgaggcagcagccttcgtggcaagaactagcgtgagcccagccaggcgcctccccaccgggctctcaggacaccctgccacaccccacggggcttgggcgactacagggtcttcgggccccagccctggagctgcaggagttgggaacggggcttttgtttccgttgctgtttatcgatgctggttttcagaattcctaggtt

SEQ ID NO: 108 - DNA, 1179 bp, Homo sapiens
  misc_feature: Incyte ID No 7504125CB1
acggctgtcagcatggaaagtcgggggctttcgcccgggtcctcctagaaattccccccgaagaagactcccccacatctgggtatggagagtgcaatcacgctgtggcagttcctgttgcagttgctgctggatcagaaacatgagcatttgatctgctggacctcgaacgatggtgaattcaagctcctcaaagcagaagaagtggccaagctgtggggactccgaaaaaacaaaacaaatatgaactatgataagctgagcagagccctgcgatactattatgacaagacaccaaatggattgcttctgactccgagtccactgctctccagcatacatttctggagcagccttagtccagttgctccgctgagtcctgccaggctgcaagggccaagcacgctgttccagttccccacactgcttaatggccacatgccagtgccaatccccagtctggacagagctgcttctccagtactgctttcttcaaactctcagaaatcctgatgacgtctggccacaattaaggactcattaactgatgaaacaaatttgtccccacgggctagtttacctgtgtcgtgagaaggacattgtgaaactcttgttaatttggtttgcacttttcataacatggatagtctagatttatgttagcattttaaaaactgtttttgatatattcaagtatatatgaaaatctgtttggcattaagtgaattttaatgtttttgtttttatatccttttagctcttaagtgttgaacactgttgacagtgaagaacttttcttaatggttttcagtataactaataaggatgtgaagcttttttctctttagttctgagtatgctaaactgtgtgcttatatagactataaccagttgtgccttccttcgcatttaatgtaaatgaatgatttatatattttttagtattaagaggaaatgtttgaaagatgaaaattagtatcaaacagctctctagtagaatttcattatttttcaccagtgggcaatatgaaagcatatatcacgttttgttttactttcaattgtataagaattgccttagaacctcttttgaactgaaattcagtaaatgtccaagtaatgtttttataataaactaagccatatttagacaataaaaaaaaaaaaaaaaaaaggggg

SEQ ID NO: 109 - DNA, 2622 bp, Homo sapiens
  misc_feature: Incyte ID No 7505742CB1
tttttttttaattcctgaggggtggttgctgctttcgctacatgacttgccagcgcccgagcctgcggtccaactgcgctgctgccggagcgctcagtgccgccgctgccgcccgcgccccccgcgccccgttcggcacccaccggtcgccgccgcccgccgcgccgctgtcccgctcccgcgccgccgccgccgtttccccccgacgactgggtgatgctggacatgggagataggaaagaggtgaaaatgatccccaagtcctcgttcagcatcaacagcctggtgcccgaggcggtccagaacgacaaccaccacgcgagccacggccaccacaacagccaccacccccagcaccaccaccaccaccaccaccatcaccaccacccgccgccgcccgccccgcaaccgccgccgccccgagccgcgcagcagcagcagccgccgccgccgccgctcgccccgcaggccggcggcgccgcgcaatcgaacgacgaaaagggcccccagctgcttctgctcccgccgaccgaccaccaccggccgccgtccggagctaaagccggaggctgctgccggccgggggagctggcgcccgtcgggccggacgagaaggagaagggcgccggcgccgggggggaggagaagaagggggcgggcgagggcggcaaggacggggaggggggcaaggagggcgagaagaagaacggcaagtacgagaagccgccgttcagctacaacgcgctcatcatgatggccatccggcagagccccgagaagcggctcacgctcaacggcatctacgagttcatcatgaagaacttcccttactaccgcgagaacaagcagggctggcagaactccatccgccacaatctgtccctcaacaagtgcttcgtgaaggtgccgcgccactacgacgacccgggcaagggcaactactggatgctggacccgtcgagcgacgacgtgttcatcggcggcaccacgggcaagctgcggcgccgctccaccacctcgcgggccaagctggccttcaagcgcggtgcgcgcctcacctccaccggcctcaccttcatggaccgcgccggctccctctactggcccatgtcgcccttcctgtccctgcaccacccccgcgccagcagcactttgagttacaacggcaccacgtcggcctaccccagccaccccatgccctacagctccgtgttgactcagaactcgctgggcaacaaccactccttctccaccgccaacggcctgagcgtggaccggctggtcaacggggagatcccgtacgccacgcaccacctcacggccgccgcgctagccgcctcggtgccctgcggcctgtcggtgccctgctctgggacctactccctcaacccctgctccgtcaacctgctcgcgggccagaccagttactttttcccccacgtcccgcacccgtcaatgacttcgcagagcagcacgtccatgagcgccagggccgcgtcctcctccacgtcgccgcaggccccctcgaccctgccctgtgagtctttaagaccctctttgccaagttttacgacgggactgtctgggggactgtctgattatttcacacatcaaaatcaggggtcttcttccaaccctttaatacattaacatccctgggaccagactgtaagtgaacgttttacacacatttgcattgtaaatgataattaaaaaaataagtccaggtattttttattaagcccccccctcccatttctgtacgtttgttcagtctctagggttgtttattattctaacaaggtgtggagtgtcagcgaggtgcaatgtggggagaatacattgtagaatataaggtttggaagtcaaattatagtagaatgtgtatctaaatagtgactgctttgccatttcattcaaacctgacaagtctatctctaagagccgccagatttccatgtgtgcagtattataagttatcatggaactatatggtggacgcagaccttgagaacaacctaaattatggggagaattttaaaatgttaaactgtaatttgtatttaaaaagcattcgtagtaaaggtgcccaagaaattattttggccatttattgttttgtccttttctttaaagaactgtttttttttcttttgtttacttttagaccaaagattgggttctagaaaatgcacttggtatactaagtattaaaacaaacaaaaaggaaagttgtttcagttggcaacactgcccattcaattgaatcagaaggggacaaaattaacgattgccttcagtttgtgttgtgtatattttgatgtatgtggtcactaacaggtcacttttattttttctaaatgtagtgaaatgttaatacctattgtacttataggtaaaccttgcaaatatgtaacctgtgttgcgcaaatgccgcataaatttgagtgattgttaatgttgtcttaaaatttcttgattgtgatactgtggtcatatgcccgtgtttgtcacttacaaaaatgtttactatgaacacacagaaataaaaaataggctaaattcatataaaaaaaaaaaaaaaaaaaaaaaaaaaaaaattctgggcgcaagaattcgctg

SEQ ID NO: 110 - DNA, 4688 bp, Homo sapiens
  misc_feature: Incyte ID No 7505757CB1
atgagcaccgccgccttccacatctccagcctcctggagaagatgacgtccagcgacaaggacttcaggtgcaagcccccccacattaatagagcctgggatccaaggtcttggcttcagatcccagctgccgtcactcactggccgtctgctcagcaatctcctccaccctccctgtgcaggttcatggccaccagcgacctgatgtcggagttgcagaaggactccatccagctggacgaggacagcgagcgcaaggtggtgaagatgctgctccggctcctggaggacaagaacggtgaggtgcagaacctggctgtcaagtgcctgggtcctctggtggtcaaagtgaaggagtaccaggtggagaccattgtggacaccctgtgcaccaacatgcggtcagacaaggagcagctgcgagacattgccggcattggcctcaagaccgtcctctcggagctccctcctgcagccacaggctccgggctggccaccaacgtgtgccggaagatcacaggccagctcaccagtgccattgcccagcaggaggatgtggctgtgcagctggaagccctggacatcctctctgacatgctgagcaggctgggtgtcccgctgggcgccttccacgccagcctcctgcactgtctgctgccacagctgagcagcccgcgcctggcggtgcgcaagcgggcggtcggagcgcttggccacctggcggccgcctgcagcaccgacctcttcgtcgagctcgctgaccacctactggaccggctgcccggcccgcgggtgcccaccagcccgactgccatccgcaccctgatccaatgtttgggcagcgtcggccgccaggccggccaccgcctcggggctcacctggaccgcctggtgcccctggtggaggatttctgcaacctggatgatgatgagctccgggagtcctgcctccaggcttttgaggccttcttgaggaagtgccccaaggaaatgggtcctcacgtgcccaacgtgaccagcctctgcctccaatacataaaacacgaccccaactacaactacgacagtgatgaggatgaggagcagatggagacagaggatagtgaattcagtgagcaagagagtgaagacgagtacagcgatgacgatgacatgagctggaaggtgcgccgggcagctgccaagtgcatcgcagccttgatcagctcgcggcctgacctgctgcccgatttccactgcaccctggcacctgtgctcatccgccgcttcaaagaacgcgaggagaacgtcaaggctgacgtcttcactgcttacatcgtgctgctgcggcaaacacagcccccgaagggatggctggaggccatggaggaacccacccagaccggcagcaacctccatatgctacgtggacaggtgccccttgtggtcaaggccctgcagcggcagcttaaagatcggagcgtcagagcccgccagggatgcttcagcctcctcaccgagctggcgggtgtcctcccaggcagcctggccgagcatatgcctgtgctggtatcaggcatcatcttctcgctggccgaccgctccagctcctccaccatccggatggatgccctggccttcttgcaggggctgctgggcaccgaaccagctgaggccttccacccacacttgcctatcctcctgccacctgtgatggcctgtgtggctgactctttctacaagattgcagccgaggccctggtggtgctgcaggagctggtgcgggccctgtggccgctgcacaggcctcggatgctggatcctgagccatatgttggagagatgtctgctgtcaccctggcgcgacttcgtgccactgacctggaccaggaggtgaaggagcgggccatttcctgcatgggccaccttgtaggccacctgggtgaccggcttggggatgacctggagcccacgttactgctcctcctggaccgcctgcggaatgagatcacccggctgcccgccatcaaggcgcttacgctggtggccgtatccccactacagcttgacctacagcccatcctggccgaggcactgcacattctggcctcattcctgcggaagaaccagcgggctttgcgactggccacactggcagccctggacgccctggcccagagccagggcctcagcctcccaccgtctgccgtgcaggccgtgctggctgagctgcctgccctggtcaacgagagcgacatgcatgtggcccagctggctgtggacttccttgccacagtgacccaggcccagccagcctctttggtggaggtcagtggccctgtgctctcagagctgctgcggctgctgcgttcgcccctgttgccagccggggttctggcagctgctgaaggcttcctgcaggccctggtagggacccgtcccccgtgtgtggactatgccaaactcatcagcctgctcactgcgcctgtttatgagcaggctgtggatggtgggcctggcctgcacaagcaggtgttccactcattggcccggtgtgtggcagccctctcagctgcctgtccccaagaggcggcaagcacagccagtcgcctggtctgcgatgccaggtcgccccactccagcacgggggtcaaggtcctggcattcttgtcgctggctgaggtgggtcaggtggctgggccaggcccccagcgggagctgaaggcggtgctcctggaagctttggggtcacccagtgaggatgtgagggctgcagcctcgtatgcactgggccgtgtgggtgctggcagcctgcccgacttcctgcccttcctgctggagcagatcgaggctgagccccgacgacagtacctgctgctgcactcactcagggaggccctgggggccgcccagcctgacagcctgaagccctacgccgaggacatctgggccttgctgttccagcgctgcgagggtgctgaggagggcacccggggggtggtggccgagtgcattgggaagctggtccttgtgaacccttcgttccttctgccccgcttgcggaagcagcttgctgcaggtcggccacacacccggagcaccgtcatcacagcggtcaagttccttatctcggaccagccccatcccattgaccccctcctgaagagcttcatcggagagttcatggagagcctgcaggacccagacctgaacgtgcgccgtgcgactctggctttcttcaactcagctgtgcacaacaagccctcgctagtccgggacctgctggatgacatcctgcccctcctctaccaggagacaaagatccggcgggacctcatccgagaggtggagatggggccctttaaacatacagtggacgatgggctggacgtgcggaaggcggcctttgaatgcatgtattcactgcttgagagctgcctgggccagctggatatctgtgagttcctgaaccatgtggaggacgggctgaaggaccactacgacatccggatgctgaccttcatcatggttgcccggctggccaccctgtgtcctgcacctgtcctgcagagggtggaccgactcattgagccactaagggccacctgcactgccaaggtcaaagctggttctgtgaagcaggagtttgaaaagcaagatgaactgaagcgctctgcaatgagggcagtggctgccctgctgaccatccccgaggtggggaaaagccccatcatggccgacttctcttcccaaatcagatccaaccctgaacttgctgccctctttgaaagcatccagaaggattccacttcagcccccagcacagactcaatggagctcagctagtcccctcagcaccaaggtgggccctcgcttaagagaaaggagcccacccaagtccgaggcctccccatcccaccatcgcaggtctctacttttgcccttccaccatctcactgggggccctgtcgctcctggtcagggcttacagtgccttctccagggacccaactcaaaggcccccagcccaagctgtgaggctgccaacagttgggccccttccttaactcaggacagtcatccaaagaaatagggtgaggaagttttccagtgacttcacactgtacccctccatagtctgtctggttccttcagagggtgtctctgcctcacaaactagtagtatttagaaataggctgtgctgtcagctgtaaaagatcaggaggcagcagacaccactctggtttcttcactgcattcagcaatgcctgaagttagtgctcaggccgggcatctcaaaagaaaagatacttgagttattcacattttaaaattcaaaacggttcatttttaagtggcagtgatgaatcagaaatttggaagatgatacgggtttcttttttccagggaggaggaatgggttgggtagggaactggacaggcttggacctcatgtttcatttctaatttcaaaatacttattagcaaattgggcaacaatgggcatcttccatgccaccacccaggcataaccagttggtttgtttccttctgaggaaggtttcaaatgtgtctagtgttcagtattgaggacaaagaaatacaagtggcaggcccaagtattttctgtgatatcccaggttaataaagattagattctaagttacttctttcctcaaaaaaaaaaaaaaaaaaaaaaaaaattggt

SEQ ID NO: 111 - DNA, 490 bp, Homo sapiens
  misc_feature: Incyte ID No 7504126CB1
gggcggcgcgtggtctacgccgagtgacagagacgctcaggctgtgttctcaggatgaccgagtgggagacagcagcaccagcggtggcagagaccccagacatcaagctctttgggaagtggagcaccgatgatgtgcagatcaatgacatttccctgcaggccatctggctgctgtgcacaggcgctcgtgaggctgccttccggaacattaagaccattgctgagtgcctggcagatgagctcatcaatgctgccaagggctcctcgaactcctatgccattaagaagaaggacgagctggagcgtgtggccaagtccaaccgctgattttcccagctgctgcccaataaacctgtctgccctttggggcagtcccagcaaaaaaaaaaaaaaaaaaaaaaaaaaaaaaaaaaaaaaaaaaaaaaaaaaaaaaaaaaaaaaaaaggggcggccgttttaggggttccaagtttacgtacgggtgcat

SEQ ID NO: 112 - DNA, 1408 bp, Homo sapiens
  misc_feature: Incyte ID No 7504099CB1
ccacgcgtgcgggcgctagatccgctgctgctgccgcggcgggcggacctgcaggagcgcggcggcggcggcggcggccgaggctgaaggaagatggcggacggcgtggaccacatagacatttacgcggatgtcggcgaagagttcaaccaggaagctgaatatggtgggcatgatcagatagatttgtatgacgatgtcatatctccatctgcaaataatggagatgccccagaagaccgagattacatggatactccacctccagttccaggctacggcccccctcctggcccaccacctccacaacagggaccacctccacctccaggcccctttccacctcgtccacccggtccacttgggccaccccttacactagctcctcctccgcatcttcctggaccacctccaggtgccccaccgccagctccgcatgtgaacccagctttctttcctccaccaactaacagtggcatgcctacatcagatagccgaggtccaccaccaacagatccatatgggcgacctccaccatatgataggggtgactatggcccccctggaagggaaatggatactgcaagaacgccattgagtgaagctgaatttgaagaaatcatgaatagaaatagggcaatctcaagcagtgctatttcgagagctgtgtctgatgccagtgctggtgattatgggagtgctattgagacactggtaactgcaatttctttaattaaacaatccaaagtatctgctgatgatcgttgcaaagttcttattagttctttgcaagattgccttcatggaattgagtccaagtcttatggttctggatcaagaagacgtgaacgatcaagagagagggaccatagtagatcacgagaaaagagtcgacgtcataaatcccgtagtagagaccgtcatgacgattattacagagagagaagcagagaacgagagaggcaccgggatcgtgaccgagaccgtgaccgagagcgtgaccgagagcgcgaatatcgtcatcgttagaagctgaaggaagaggatcaccttccaagacaaaacagtcttcatgggggaaaaatgacgcttgtccagcagtttgcttcttgtgattgaactgaacctgtaaggattcatggataaaatgaacaggaatagatctgaataaagcaaatctgcataaatggtaaccagtagctctacttttattttttatgttgcttaactggtttatttgaaggaaacctgtgtgatttaaaaagttatagcttttgcaactttattactggttatatacatttggccattatgatgtgcaagcaattggaaaaaaagtcaagtaaatgcctggttttgtcgtaggttggtctggtaaaatcgttatatgatatgtctgtgacagcta

SEQ ID NO: 113 - DNA, 1363 bp, Homo sapiens
  misc_feature: Incyte ID No 7505733CB1
gctcaggccttctgagcccaacccaagccatcgcatcccctgtgacttgcacgtatatgcctagatggcctgaagtaactgaagaatcacaaaagaagtgaatatgccctgccccgccttaactgatgacattccaccaccaaagaagtgaaaaaggccggtccttgacttaactgatgacattatcttatgaaattccttctcctggctcatcctggctcaaaaagctcccccactgagcacgttgccacccccactcctgcccgccagagaacaaaccccctttttcctttacctacccaaatcttataaaacggcctcacgcctatctccctttgctgactcccttttcggactcagcccacctgcgcccaggtgaaataaacagccttgttgctcacacaaagcctgtttggtggtctcttcacacggacgtgcatgaaatttggtgccgtaactggcgcggggggagggggggggggacctcccttgggagatcaatcccctgtcctcctgctctttgctccatgagaaagatccacctatgacctcaggtcctcagaccgaccagcccaagaaacatctcaccaatttcaaatcggattcccaactatatgaagacaccctagctggacgatcagttcttattaagaacctgactcctcaaactctacaacctcgatggaccggaccctacttagtcatctatagtaccccgactgctgtccgcctgcaggatcctccccactgggttcaccgttccagaataaagctgtgtccatcggacagccagcctaatccctcctcttcctcctggaagttgcaagtactctcccctacttcccttaaactcagtcgtatttctgaagaacagtaataacccttatgagcctaatacatcccttcattctattaggtctgttcgtccttaccctactttttgcaacagggctttacgaagtcaccccaccacttaggccgagccccaagaaactagtcatccctactatcttctgtctggtcatactcctattctccattctcaactacttataaatgccctactcttgtttacacggacggtttacactgtttctccaagccatcacagctgatatctcttagtgctatccccaaactgccactcttaactccctcttagagtggatagatgatctttgctggcaaggcaccctccaatacttccaccctgatgaagttctattctttacttttatactcactcttattctcattcccattcttatgtcaccctctacctctccccagctatctccaccacactatcaaccttacccattctctcctagacgtttctaatccctccttagcgaacaactgc

SEQ ID NO: 114 - DNA, 1071 bp, Homo sapiens
  misc_feature: Incyte ID No 7959829CB1
cgcggcgcggccggctgccggaaaacagggcagacctgtatgattggtttattcctggggttgtcatatcatggctgataatgacacagacagaaaccagactgagaagctcctaaaaagagtacgagaactggagcaagaggtgcaaagacttaaaaaggaacaggccaaaaataaggaggactcaaacattagagaaaattcagcaggagctggaaaaactaagcgtgcatttgatttcagtgctcatggccgaagacacgtagccctaagaatagcctatatgggctggggataccagggctttgctagtcaggaaaacacaaataataccattgaagagaaactgtttgaagctctaaccaagactcgactagtagaaagcagacagacatccaactatcaccgatgtgggagaacagataaaggagttagtgcctttggacaggtgatctcacttgaccttcgctctcagtttccaaggggcagggattccgaggactttaatgtaaaagaggaggctaatgctgctgctgaagagatccgttatacccacattctcaatcggtatggctgtagaatttcctctagtcttatatgactgtaagtttgaaaatgtcaagtggatctatgaccaggaggctcaggagttcaatattacccacctacaacaactgtgggctaatcatgctgtcaaaactcacatgttgtatagtatgctacaaggactggacactgttccagtaccctgtggaataggaccaaagatggatggaatgacagaatggggaaatgttaagccctctgtcataaagcagaccagtgccttagtagaaggagtgaagatgcgcacatataagcccctcatggaccgtcctaaatgccaaggactggaatcccggatccagcattttgtacgtcaggggacgaattgagcacccacatttattccatgaggaagaaacaaaagccaaaagggactgtaatgacacactagaggaagagaatactaatttggagacaccaacgaagaggttctgtgttgacacaaaaatttaacgcagtcaggta

SEQ ID NO: 115 - DNA, 2140 bp, Homo sapiens
  misc_feature: Incyte ID No 7502168CB1
gccggcggggcgcgcggcggtgcgggcgggtgactggcggcgggcgccgcggtcgggctggctgccgggcagcatggaggagctgagcagcgtgggcgagcaggtcttcgccgccgagtgcatcctgagcaagcggctccgcaagggcaagctggagtacctggtcaagtggcgcggctggtcctccaaacataacagctgggagccggaggagaacatcctggacccgaggctgctcctggccttccagaagaaggaacatgagaaggaggtgcagaaccggaagagaggcaagaggccgagaggccggccaaggaagctcactgccatgtcctcctgcagccggcgctccaagctcaaggaacccgatgctccctccaaatccaagtccagcagttcctcctcttcctccacgtcatcctcctcttcctcagatgaagaggatgacagtgacttagatgctaagaggggtccccggggccgcgagacccacccagtgccgcagaagaaggcccagatcctggtggccaaacccgagctgaaggatcccatccggaagaagcggggacgaaagcccctgcccccagagcaaaaggcaacccgaagacccgtgagcctggccaaggtgctgaagaccgcccggaaggatctgggggccccggccagcaagctgccccctccactcagcgcccccgttgcaggcctggcagctctgaaggcccacgccaaggaggcctgtggcggccccagtgccatggccaccccagagaacctggccagcctaatgaagggcatggccagtagccccggccggggtggcatcagctggcagagctccatcgtgcactacatgaaccggatgacccagagccaggcccaggctgccagcaggttggcgctgaaggcccaggccaccaacaagtgcggcctcgggctggacctgaaggtgaggacgcagaaaggggagctgggaatgagccctccaggaagcaaaatcccgaaggcccccagcggtggggctgtggagcagaaagtggggaacacagggggccccccgcacacccatggtgccagcagggtgcctgctgggtgcccaggcccccagccagcacccacccaggagctgagcctccaggtcttggacttgcagagtgtcaagaatggcatgcccggggtgggtctccttgcccgccacgccaccgccaccaagggtgtcccggccaccaacccagcccctgggaagggcactgggagtggcctcattggggccagcggggccaccatgcccaccgacacaagcaaaagtgagaagctggcttccagagcagtggcgccacccacccctgccagcaagagggactgtgtcaagggcagtgctacccccagtgggcaggagagccgcacagcccccggagaagcccgcaaggcggccacactgccagagatgagcgcaggtgaggagagtagcagctcggactccgaccccgactccgcctcgccgcccagcactggacagaacccgtcagtgtccgttcagaccagccaggactggaagcccacccgcagcctcatcgagcacgtatttgtcacctgcttccctaccactcctcactgcattttccatacaaatgtttctattttattgttccttcttgtaataaagggaagataaaaccatccttagcgctgtctccctcaatatcccccaccccatcttgttgtgcaaactgactgcttgatttgggggtgcctggcctttgaggtagtcacagggaggcccctccccaacatgagactgggtggggatggggagagagaagtggggaatggaggggaaggtgcttggggaatttctttgtccagggtgccccatctagccttccggccctttggaaccctttctgcgctttgctggtggctcctgagcatggcgggattggcgcaggtcggcactgaacagcacctgtaggagggtggagtctgtgtggggaggagggtacactggggtcagggctggtgagactagtgacagtgttgggaggtggaagagtccttggggaacagggccgaaggcaatgagaatccactggggttgggacaggggtggctggagagtcctttagggccacctggggcggtg

SEQ ID NO: 116 - DNA, 4980 bp, Homo sapiens
  misc_feature: Incyte ID No 7503888CB1
ggcgcgcgtgtgtgtgaagggggggcggtggccgaggcgggcgggcgcgcgcgcgaggcttcccctcgtttggcggcggcggcggcttctttgtttcgtgaagagaagcgagacgcccattctgcccccggccccgcgcggaggggcgggggaggcgccgggaagtcgacggcgccggcggctcctgcaggaggccactgtctgcagctcccgtgaagatgtccactccagacccacccctgggcggaactcctcggccaggtccttccccgggccctggcccttcccctggagccatgctgggccctagcccgggtccctcgccgggctccgcccacagcatgatggggcccagcccagggccgccctcagcaggacaccccatccccacccaggggcctggagggtaccctcaggacaacatgcaccagatgcacaagcccatggagtccatgcatgagaagggcatgtcggacgacccgcgctacaaccagatgaaaggaatggggatgcggtcagggggccatgctgggatggggcccccgcccagccccatggaccagcactcccaaggttacccctcgcccctgggtggctctgagcatgcctctagtccagttccagccagtggcccgtcttcggggccccagatgtcttccgggccaggaggtgccccgctggatggtgctgacccccaggccttggggcagcagaaccggggcccaaccccatttaaccagaaccagctgcaccagctcagagctcagatcatggcctacaagatgctggccagggggcagcccctccccgaccacctgcagatggcggtgcagggcaagcggccgatgcccgggatgcagcagcagatgccaacgctacctccaccctcggtgtccgcaacaggacccggccctggccctggccctggccccggcccgggtcccggcccggcacctccaaattacagcaggcctcatggtatgggagggcccaacatgcctcccccaggaccctcgggcgtgccccccgggatgccaggccagcctcctggagggcctcccaagccctggcctgaaggacccatggcgaatgctgctgcccccacgagcacccctcagaagctgattcccccgcagccaacgggccgcccttcccccgcgccccctgccgtcccacccgccgcctcgcccgtgatgccaccgcagacccagtcccccgggcagccggcccagcccgcgcccatggtgccactgcaccagaagcagagccgcatcacccccatccagaagccgcggggcctcgaccctgtggagatcctgcaggagcgcgagtacaggctgcaggctcgcatcgcacaccgaattcaggaacttgaaaaccttcccgggtccctggccggggatttgcgaaccaaagcgaccattgagctcaaggccctcaggctgctgaacttccagaggcagctgcgccaggaggtggtggtgtgcatgcggagggacacagcgctggagacagccctcaatgctaaggcctacaagcgcagcaagcgccagtccctgcgcgaggcccgcatcactgagaagctggagaagcagcagaagatcgagcaggagcgcaagcgccggcagaagcaccaggaatacctcaatagcattctccagcatgccaaggatttcaaggaatatcacagatccgtcacaggcaaaatccagaagctgaccaaggcagtggccacgtaccatgccaacacggagcgggagcagaagaaagagaacgagcggatcgagaaggagcgcatgcggaggctcatggctgaagatgaggaggggtaccgcaagctcatcgaccagaagaaggacaagcgcctggcctacctcttgcagcagacagacgagtacgtggctaacctcacggagctggtgcggcagcacaaggctgcccaggtcgccaaggagaaaaagaagaaaaagaaaaagaagaaggcagaaaatgcagaaggacagacgcctgccattgggccggatggcgagcctctggacgagaccagccagatgagcgacctcccggtgaaggtgatccacgtggagagtgggaagatcctcacaggcacagatgcccccaaagccgggcagctggaggcctggctcgagatgaacccggggtatgaagtagctccgaggtctgatagtgaagaaagtggctcagaagaagaggaagaggaggaggaggaagagcagccgcaggcagcacagcctcccaccctgcccgtggaggagaagaagaagattccagatccagacagcgatgacgtctctgaggtggacgcgcggcacatcattgagaatgccaagcaagatgtcgatgatgaatatggcgtgtcccaggcccttgcacgtggcctgcagtcctactatgccgtggcccatgctgtcactgagagagtggacaagcagtcagcgcttatggtcaatggtgtcctcaaacagtaccagatcaaaggtttggagtggctggtgtccctgtacaacaacaacctgaacggcatcctggccgacgagatgggcctggggaagaccatccagaccatcgcgctcatcacgtacctcatggagcacaaacgcatcaatgggcccttcctcatcatcgtgcctctctcaacgctgtccaactgggcgtacgagtttgacaagtgggccccctccgtggtgaaggtgtcttacaagggatccccagcagcaagacgggcctttgtcccccagctccggagtgggaagttcaacgtcttgctgacgacgtacgagtacatcatcaaagacaagcacatcctcgccaagatccgttggaagtacatgattgtggacgaaggtcaccgcatgaagaaccaccactgcaagctgacgcaggtgctcaacacgcactatgtggcaccccgccgcctgctgctgacgggcacaccgctgcagaacaagcttcccgagctctgggcgctgctcaacttcctgctgcccaccatcttcaagagctgcagcaccttcgagcagtggtttaacgcaccctttgccatgaccggggaaaaggtggacctgaatgaggaggaaaccattctcatcatccggcgtctccacaaagtgctgcggcccttcttgctccgacgactcaagaaggaagtcgaggcccagttgcccgaaaaggtggagtacgtcatcaagtgcgacatgtctgcgctgcagcgagtgctctaccgccacatgcaggccaagggcgtgctgctgactgatggctccgagaaggacaagaagggcaaaggcggcaccaagaccctgatgaacaccatcatgcagctgcggaagatctgcaaccacccctacatgttccagcacatcgaggagtccttttccgagcacttggggttcactggcggcattgtccaagggctggacctgtaccgagcctcgggtaaatttgagcttcttgatagaattcttcccaaactccgagcaaccaaccacaaagtgctgctgttctgccaaatgacctccctcatgaccatcatggaagattactttgcgtatcgcggctttaaatacctcaggcttgatggaaccacgaaggcggaggaccggggcatgctgctgaaaaccttcaacgagcccggctctgagtacttcatcttcctgctcagcacccgggctggggggctcggcctgaacctccagtcggcagacactgtgatcatttttgacagcgactggaatcctcaccaggacctgcaagcgcaggaccgagcccaccgcatcgggcagcagaacgaggtggagcggctgacctgtgaggaggaggaggagaagatgttcggccgtggctcccgccaccgcaaggaggtggactacagcgactcactgacggagaagcagtggctcaaggccatcgaggagggcacgctggaggagatcgaagaggaggtccggcagaagaaatcatcacggaagcgcaagcgagacagcgacgccggctcctccaccccgaccaccagcacccgcagccgcgacaaggacgacgagagcaagaagcagaagaagcgcgggcggccgcctgccgagaaactctcccctaacccacccaacctcaccaagaagatgaagaagattgtggatgccgtgatcaagtacaaggacagcagcagtggacgtcagctcagcgaggtcttcatccagctgccctcgcgaaaggagctgcccgagtactacgagctcatccgcaagcccgtggacttcaagaagataaaggagcgcattcgcaaccacaagtaccgcagcctcaacgacctagagaaggacgtcatgctcctgtgccagaacgcacagaccttcaacctggagggctccctgatctatgaagactccatcgtcttgcagtcggtcttcaccagcgtgcggcagaaaatcgagaaggaggatgacagtgaaggcgaggagagtgaggaggaggaagagggcgaggaggaaggctccgaatccgaatctcggtccgtcaaagtgaagatcaagcttggccggaaggagaaggcacaggaccggctgaagggcggccggcggcggccgagccgagggtcccgagccaagccggtcgtgagtgacgatgacagtgaggaggaacaagaggaggaccgctcaggaagtggcagcgaagaagactgagccccgacattccagtctcgaccccgagcccctcgttccagagctgagatggcataggccttagcagtaacgggtagcagcagatgtagtttcagacttggagtaaaactgtataaacaaaagaatcttccatatttatacagcagagaagctgtaggactgtttgtgactggccctgtcctggcatcagtagcatctgtaacagcattaactgtcttaaagagagagagagagaattccgaattggggaacacaaaaaaaaaaaaaaaaaaaaaagggcggc